SEQUENCE LISTING (137 sequences)

SEQ ID NO: 1 - protein, 4 aa, synthetic construct
GGFG

SEQ ID NO: 2 - protein, 4 aa, synthetic construct
ALAL

SEQ ID NO: 3 - protein, 4 aa, synthetic construct
GFLG

SEQ ID NO: 4 - RNA, 21 nt, synthetic construct
  source 1..21: mol_type = other RNA
ttattatttgttctttgcctt

SEQ ID NO: 5 - RNA, 21 nt, synthetic construct
  source 1..21: mol_type = other RNA
ttacattaaagtctgttgttt

SEQ ID NO: 6 - RNA, 21 nt, synthetic construct
  source 1..21: mol_type = other RNA
taataccgaggaatgtcggtt

SEQ ID NO: 7 - RNA, 21 nt, synthetic construct
  source 1..21: mol_type = other RNA
ttcgaagacagttctagggtt

SEQ ID NO: 8 - skipped

SEQ ID NO: 9 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
taataccgaggaatgtcggtt

SEQ ID NO: 10 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
taataccgaggaatgtcggtt

SEQ ID NO: 11 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
taataccgaggaatgtcggtt

SEQ ID NO: 12 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
taataccgaggaatgtcggtt

SEQ ID NO: 13 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttacattaaagtctgttgttt

SEQ ID NO: 14 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttattatttgttctttgcctt

SEQ ID NO: 15 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..2: mod_base = OTHER; note = phosphorothioate linkage
  modified_base 2..3: mod_base = OTHER; note = phosphoryl guanidine linkage
  modified_base 19..20: mod_base = OTHER; note = phosphoryl guanidine linkage
  modified_base 20..21: mod_base = OTHER; note = phosphorothioate linkage
ttattatttgttctttgcctt

SEQ ID NO: 16 - RNA, 21 nt, synthetic construct
  source 1..21: mol_type = other RNA
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
taataccgaggaatgtcggtt

SEQ ID NO: 17 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 18 - RNA, 21 nt, synthetic construct
  source 1..21: mol_type = other RNA
  modified_base 1: mod_base = OTHER; note = (2R,3S,4R,5R)-3-(hexadecyloxy)-5-(hydroxymethyl)tet rahydrofuran-2,4-diol nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 19 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = (2R,3S,4R,5R)-3-(hexadecyloxy)-5-(hydroxymethyl)tet rahydrofuran-2,4-diol nucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 20 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = (2R,3S,4R,5R)-3-(hexadecyloxy)-5-(hydroxymethyl)tet rahydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 21 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = (2R,3S,4R,5R)-3-(hexadecyloxy)-5-(hydroxymethyl)tet rahydrofuran-2,4-diol nucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 22 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = (2R,3S,4R,5R)-3-(hexadecyloxy)-5-(hydroxymethyl)tet rahydrofuran-2,4-diol nucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 23 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexadecyloxy)-5-(hydroxymethyl)tet rahydrofuran-2,4-diol nucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 24 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = (2R,3S,4R,5R)-3-(hexadecyloxy)-5-(hydroxymethyl)tet rahydrofuran-2,4-diol nucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 25 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = (2R,3S,4R,5R)-3-(hexadecyloxy)-5-(hydroxymethyl)tet rahydrofuran-2,4-diol nucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 26 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = (2R,3S,4R,5R)-3-(hexadecyloxy)-5-(hydroxymethyl)tet rahydrofuran-2,4-diol nucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 27 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = (2R,3S,4R,5R)-3-(hexadecyloxy)-5-(hydroxymethyl)tet rahydrofuran-2,4-diol nucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 28 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = (2R,3S,4R,5R)-3-(hexadecyloxy)-5-(hydroxymethyl)tet rahydrofuran-2,4-diol nucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 29 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = (2R,3S,4R,5R)-3-(hexadecyloxy)-5-(hydroxymethyl)tet rahydrofuran-2,4-diol nucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 30 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = (2R,3S,4R,5R)-3-(hexadecyloxy)-5-(hydroxymethyl)tet rahydrofuran-2,4-diol nucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 31 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = (2R,3S,4R,5R)-3-(hexadecyloxy)-5-(hydroxymethyl)tet rahydrofuran-2,4-diol nucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 32 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = (2R,3S,4R,5R)-3-(hexadecyloxy)-5-(hydroxymethyl)tet rahydrofuran-2,4-diol nucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 33 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = (2R,3S,4R,5R)-3-(hexadecyloxy)-5-(hydroxymethyl)tet rahydrofuran-2,4-diol nucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 34 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = (2R,3S,4R,5R)-3-(hexadecyloxy)-5-(hydroxymethyl)tet rahydrofuran-2,4-diol nucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 35 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = (2R,3S,4R,5R)-3-(hexadecyloxy)-5-(hydroxymethyl)tet rahydrofuran-2,4-diol nucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 36 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = (2R,3S,4R,5R)-3-(hexadecyloxy)-5-(hydroxymethyl)tet rahydrofuran-2,4-diol nucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 37 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = (2R,3S,4R,5R)-3-(hexadecyloxy)-5-(hydroxymethyl)tet rahydrofuran-2,4-diol nucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 38 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = (2R,3S,4R,5R)-3-(hexadecyloxy)-5-(hydroxymethyl)tet rahydrofuran-2,4-diol nucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 39 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 18..21: mod_base = OTHER; note = phosphorothioate linkage
ttcgaagacagttctagggtt

SEQ ID NO: 40 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..20: mod_base = OTHER; note = phosphorothioate linkage
ttcgaagacagttctagggtt

SEQ ID NO: 41 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = (2R,3S,4R,5R)-3-(hexadecyloxy)-5-(hydroxymethyl)tet rahydrofuran-2,4-diol nucleotide
  modified_base 1..3: mod_base = OTHER; note = phosphorothioate linkage
  modified_base 19..20: mod_base = OTHER; note = phosphorothioate linkage
ttcgaagacagttctagggtt

SEQ ID NO: 42 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = (2R,3S,4R,5R)-3-(hexadecyloxy)-5-(hydroxymethyl)tet rahydrofuran-2,4-diol nucleotide
  modified_base 1..3: mod_base = OTHER; note = phosphorothioate linkage
  modified_base 19..21: mod_base = OTHER; note = phosphorothioate linkage
ttcgaagacagttctagggtt

SEQ ID NO: 43 - RNA, 20 nt, synthetic construct
  source 1..20: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 1..3: mod_base = OTHER; note = phosphorothioate linkage
  modified_base 19..20: mod_base = OTHER; note = phosphorothioate linkage
ttcgaagacagttctagggt

SEQ ID NO: 44 - RNA, 21 nt, synthetic construct
  source 1..21: mol_type = other RNA
  modified_base 1: mod_base = OTHER; note = ((E)-4-hydroxy-3-((R)-1-hydroxy-2-(2-methoxyethoxy) ethoxy)but-1-en-1-yl)phosphonic acid nucleotide
  modified_base 2: mod_base = OTHER; note = 2'-fluoro ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-fluoro ribonucleotide
  modified_base 15: mod_base = OTHER; note = 2'-O-methyl ribonucleotide
  modified_base 16: mod_base = OTHER; note = 2'-fluoro ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 1..3: mod_base = OTHER; note = phosphorothioate linkage
  modified_base 19..21: mod_base = OTHER; note = phosphorothioate linkage
taataccgaggaatgtcggtt

SEQ ID NO: 45 - skipped

SEQ ID NO: 46 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 47 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexadecyloxy)-5-(hydroxymethyl)tet rahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 48 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 49 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexadecyloxy)-5-(hydroxymethyl)tet rahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 50 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 51 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexadecyloxy)-5-(hydroxymethyl)tet rahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 52 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(hexadecyloxy)-5-(hydroxymethyl)tet rahydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 53 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = (2R,3S,4R,5R)-3-(hexadecyloxy)-5-(hydroxymethyl)tet rahydrofuran-2,4-diol nucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 54 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = (2R,3S,4R,5R)-3-(hexadecyloxy)-5-(hydroxymethyl)tet rahydrofuran-2,4-diol nucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 55 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3R,4R,5R)-5-(hydroxymethyl)-3-((1-tetradecyl-1H -1,2,3-triazol-4-yl)methoxy)tetrahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 56 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3R,4R,5R)-3-((1-hexadecyl-1H-1,2,3-triazol-4-yl )methoxy)-5-(hydroxymethyl)tetrahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 57 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3R,4R,5R)-5-(hydroxymethyl)-3-((1-octadecyl-1H- 1,2,3-triazol-4-yl)methoxy)tetrahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 58 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3R,4R,5R)-5-(hydroxymethyl)-3-((1-icosyl-1H-1,2 ,3-triazol-4-yl)methoxy)tetrahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 59 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3R,4R,5R)-5-(hydroxymethyl)-3-((1-((Z)-octadec- 9-en-1-yl)-1H-1,2,3-triazol-4-yl)methoxy)tetrahydrofuran-2 ,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 60 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (S)-6-(4-((((2R,3R,4R,5R)-2,4-dihydroxy-5-(hydroxym ethyl)tetrahydrofuran-3-yl)oxy)methyl)-1H-1,2,3-triazol-1- yl)-2-stearamidohexanoic acid nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 61 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (5S,8S,35S)-8-acetamido-1-(4-((((2R,3R,4R,5R)-2,4-d ihydroxy-5-(hydroxymethyl)tetrahydrofuran-3-yl)oxy)methyl) -1H-1,2,3-triazol-1-yl)-7,14,23,32,37-pentaoxo-16,19,25,28 -tetraoxa-6,13,22,31,36-pentaazapentapentacontane-5,35,55- tricarboxylic acid nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 62 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(hexadecyloxy)-5-(hydroxymethyl)tet rahydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexadecyloxy)-5-(hydroxymethyl)tet rahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 63 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(docosyloxy)-5-(hydroxymethyl)tetra hydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 64 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3R,4R,5R)-5-(hydroxymethyl)-3-((1-tetradecyl-1H -1,2,3-triazol-4-yl)methoxy)tetrahydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 65 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3R,4R,5R)-3-((1-hexadecyl-1H-1,2,3-triazol-4-yl )methoxy)-5-(hydroxymethyl)tetrahydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 66 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3R,4R,5R)-5-(hydroxymethyl)-3-((1-icosyl-1H-1,2 ,3-triazol-4-yl)methoxy)tetrahydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 67 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3R,4R,5R)-5-(hydroxymethyl)-3-((1-octadecyl-1H- 1,2,3-triazol-4-yl)methoxy)tetrahydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 68 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3R,4R,5R)-5-(hydroxymethyl)-3-((1-((Z)-octadec- 9-en-1-yl)-1H-1,2,3-triazol-4-yl)methoxy)tetrahydrofuran-2 ,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 69 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (S)-6-(4-((((2R,3R,4R,5R)-2,4-dihydroxy-5-(hydroxym ethyl)tetrahydrofuran-3-yl)oxy)methyl)-1H-1,2,3-triazol-1- yl)-2-stearamidohexanoic acid nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 70 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(hexyloxy)-5-(hydroxymethyl)tetrahy drofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 71 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(decyloxy)-5-(hydroxymethyl)tetrahy drofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 72 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexyloxy)-5-(hydroxymethyl)tetrahy drofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 73 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(decyloxy)-5-(hydroxymethyl)tetrahy drofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 74 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(docosyloxy)-5-(hydroxymethyl)tetra hydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
acaacagactttaatgtaa

SEQ ID NO: 75 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ggcaaagaacaaataataa

SEQ ID NO: 76 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexadecyloxy)-5-(hydroxymethyl)tet rahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ggcaaagaacaaataataa

SEQ ID NO: 77 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = (2R,3S,4R,5R)-3-(hexadecyloxy)-5-(hydroxymethyl)tet rahydrofuran-2,4-diol nucleotide
  modified_base 1..3: mod_base = OTHER; note = phosphorothioate linkage
  modified_base 17..19: mod_base = OTHER; note = phosphorothioate linkage
ggcaaagaacaaataataa

SEQ ID NO: 78 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphoryl guanidine linkage
  modified_base 17..19: mod_base = OTHER; note = phosphoryl guanidine linkage
ggcaaagaacaaataataa

SEQ ID NO: 79 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexadecyloxy)-5-(hydroxymethyl)tet rahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphoryl guanidine linkage
  modified_base 17..19: mod_base = OTHER; note = phosphoryl guanidine linkage
ggcaaagaacaaataataa

SEQ ID NO: 80 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(docosyloxy)-5-(hydroxymethyl)tetra hydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ggcaaagaacaaataataa

SEQ ID NO: 81 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(docosyloxy)-5-(hydroxymethyl)tetra hydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 82 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = (2R,3S,4R,5R)-5-(hydroxymethyl)-3-(octadecyloxy)tet rahydrofuran-2,4-diol nucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 83 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-5-(hydroxymethyl)-3-(octadecyloxy)tet rahydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 84 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = (2R,3S,4R,5R)-5-(hydroxymethyl)-3-(icosyloxy)tetrah ydrofuran-2,4-diol nucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 85 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-5-(hydroxymethyl)-3-(icosyloxy)tetrah ydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 86 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = (2R,3S,4R,5R)-3-(docosyloxy)-5-(hydroxymethyl)tetra hydrofuran-2,4-diol nucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 87 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-5-(hydroxymethyl)-3-(octadecyloxy)tet rahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 88 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(docosyloxy)-5-(hydroxymethyl)tetra hydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ggcaaagaacaaataataa

SEQ ID NO: 89 - DNA, 41 bp, synthetic construct
  source 1..41: mol_type = other DNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 23: mod_base = OTHER; note = 2'-O-methyl ribonucleotide
  modified_base 24: mod_base = OTHER; note = 2'-O-methyl ribonucleotide
  modified_base 25: mod_base = OTHER; note = 2'-O-methyl ribonucleotide
  modified_base 26: mod_base = OTHER; note = 2'-O-methyl ribonucleotide
  modified_base 27: mod_base = OTHER; note = 2'-O-methyl ribonucleotide
  modified_base 28: mod_base = OTHER; note = 2'-O-methyl ribonucleotide
  modified_base 29: mod_base = OTHER; note = 2'-fluoro ribonucleotide
  modified_base 30: mod_base = OTHER; note = 2'-fluoro ribonucleotide
  modified_base 31: mod_base = OTHER; note = 2'-fluoro ribonucleotide
  modified_base 32: mod_base = OTHER; note = 2'-O-methyl ribonucleotide
  modified_base 33: mod_base = OTHER; note = 2'-O-methyl ribonucleotide
  modified_base 34: mod_base = OTHER; note = 2'-O-methyl ribonucleotide
  modified_base 35: mod_base = OTHER; note = 2'-O-methyl ribonucleotide
  modified_base 36: mod_base = OTHER; note = 2'-O-methyl ribonucleotide
  modified_base 37: mod_base = OTHER; note = 2'-O-methyl ribonucleotide
  modified_base 38: mod_base = OTHER; note = 2'-O-methyl ribonucleotide
  modified_base 39: mod_base = OTHER; note = 2'-O-methyl ribonucleotide
  modified_base 40: mod_base = OTHER; note = 2'-O-methyl ribonucleotide
  modified_base 41: mod_base = OTHER; note = 2'-O-methyl ribonucleotide
  modified_base 1..3: mod_base = OTHER; note = phosphorothioate linkage
  modified_base 19..23: mod_base = OTHER; note = phosphorothioate linkage
  modified_base 39..41: mod_base = OTHER; note = phosphorothioate linkage
  misc_feature 1..19: note = RNA
  misc_feature 20..22: note = DNA
  misc_feature 23..41: note = RNA
ggcaaagaacaaataataatttggcaaagaacaaataataa

SEQ ID NO: 90 - DNA, 41 bp, synthetic construct; identical to SEQ ID NO: 89
  source 1..41: mol_type = other DNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(docosyloxy)-5-(hydroxymethyl)tetra hydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 23: mod_base = OTHER; note = 2'-O-methyl ribonucleotide
  modified_base 24: mod_base = OTHER; note = 2'-O-methyl ribonucleotide
  modified_base 25: mod_base = OTHER; note = 2'-O-methyl ribonucleotide
  modified_base 26: mod_base = OTHER; note = 2'-O-methyl ribonucleotide
  modified_base 27: mod_base = OTHER; note = 2'-O-methyl ribonucleotide
  modified_base 28: mod_base = OTHER; note = 2'-O-methyl ribonucleotide
  modified_base 29: mod_base = OTHER; note = 2'-fluoro ribonucleotide
  modified_base 30: mod_base = OTHER; note = 2'-fluoro ribonucleotide
  modified_base 31: mod_base = OTHER; note = 2'-fluoro ribonucleotide
  modified_base 32: mod_base = OTHER; note = 2'-O-methyl ribonucleotide
  modified_base 33: mod_base = OTHER; note = 2'-O-methyl ribonucleotide
  modified_base 34: mod_base = OTHER; note = 2'-O-methyl ribonucleotide
  modified_base 35: mod_base = OTHER; note = 2'-O-methyl ribonucleotide
  modified_base 36: mod_base = OTHER; note = 2'-O-methyl ribonucleotide
  modified_base 37: mod_base = OTHER; note = 2'-O-methyl ribonucleotide
  modified_base 38: mod_base = OTHER; note = 2'-O-methyl ribonucleotide
  modified_base 39: mod_base = OTHER; note = 2'-O-methyl ribonucleotide
  modified_base 40: mod_base = OTHER; note = 2'-O-methyl ribonucleotide
  modified_base 41: mod_base = OTHER; note = 2'-O-methyl ribonucleotide
  modified_base 1..3: mod_base = OTHER; note = phosphorothioate linkage
  modified_base 19..23: mod_base = OTHER; note = phosphorothioate linkage
  modified_base 39..41: mod_base = OTHER; note = phosphorothioate linkage
  misc_feature 1..19: note = RNA
  misc_feature 20..22: note = DNA
  misc_feature 23..41: note = RNA

SEQ ID NO: 91 - DNA, 41 bp, synthetic construct; identical to SEQ ID NO: 89
  source 1..41: mol_type = other DNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexadecyloxy)-5-(hydroxymethyl)tet rahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 23: mod_base = OTHER; note = 2'-O-methyl ribonucleotide
  modified_base 24: mod_base = OTHER; note = 2'-O-methyl ribonucleotide
  modified_base 25: mod_base = OTHER; note = 2'-O-methyl ribonucleotide
  modified_base 26: mod_base = OTHER; note = 2'-O-methyl ribonucleotide
  modified_base 27: mod_base = OTHER; note = 2'-O-methyl ribonucleotide
  modified_base 28: mod_base = OTHER; note = 2'-O-methyl ribonucleotide
  modified_base 29: mod_base = OTHER; note = 2'-fluoro ribonucleotide
  modified_base 30: mod_base = OTHER; note = 2'-fluoro ribonucleotide
  modified_base 31: mod_base = OTHER; note = 2'-fluoro ribonucleotide
  modified_base 32: mod_base = OTHER; note = 2'-O-methyl ribonucleotide
  modified_base 33: mod_base = OTHER; note = 2'-O-methyl ribonucleotide
  modified_base 34: mod_base = OTHER; note = 2'-O-methyl ribonucleotide
  modified_base 35: mod_base = OTHER; note = 2'-O-methyl ribonucleotide
  modified_base 36: mod_base = OTHER; note = 2'-O-methyl ribonucleotide
  modified_base 37: mod_base = OTHER; note = 2'-O-methyl ribonucleotide
  modified_base 38: mod_base = OTHER; note = 2'-O-methyl ribonucleotide
  modified_base 39: mod_base = OTHER; note = 2'-O-methyl ribonucleotide
  modified_base 40: mod_base = OTHER; note = 2'-O-methyl ribonucleotide
  modified_base 41: mod_base = OTHER; note = 2'-O-methyl ribonucleotide
  modified_base 1..3: mod_base = OTHER; note = phosphorothioate linkage
  modified_base 19..23: mod_base = OTHER; note = phosphorothioate linkage
  modified_base 39..41: mod_base = OTHER; note = phosphorothioate linkage
  misc_feature 1..19: note = RNA
  misc_feature 20..22: note = DNA
  misc_feature 23..41: note = RNA

SEQ ID NO: 92 - DNA, 41 bp, synthetic construct; identical to SEQ ID NO: 89
  source 1..41: mol_type = other DNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexadecyloxy)-5-(hydroxymethyl)tet rahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 23: mod_base = OTHER; note = 2'-O-methyl ribonucleotide
  modified_base 24: mod_base = OTHER; note = 2'-O-methyl ribonucleotide
  modified_base 25: mod_base = OTHER; note = 2'-O-methyl ribonucleotide
  modified_base 26: mod_base = OTHER; note = 2'-O-methyl ribonucleotide
  modified_base 27: mod_base = OTHER; note = 2'-O-methyl ribonucleotide
  modified_base 28: mod_base = OTHER; note = (2R,3S,4R,5R)-3-(hexadecyloxy)-5-(hydroxymethyl)tet rahydrofuran-2,4-diol nucleotide
  modified_base 29: mod_base = OTHER; note = 2'-fluoro ribonucleotide
  modified_base 30: mod_base = OTHER; note = 2'-fluoro ribonucleotide
  modified_base 31: mod_base = OTHER; note = 2'-fluoro ribonucleotide
  modified_base 32: mod_base = OTHER; note = 2'-O-methyl ribonucleotide
  modified_base 33: mod_base = OTHER; note = 2'-O-methyl ribonucleotide
  modified_base 34: mod_base = OTHER; note = 2'-O-methyl ribonucleotide
  modified_base 35: mod_base = OTHER; note = 2'-O-methyl ribonucleotide
  modified_base 36: mod_base = OTHER; note = 2'-O-methyl ribonucleotide
  modified_base 37: mod_base = OTHER; note = 2'-O-methyl ribonucleotide
  modified_base 38: mod_base = OTHER; note = 2'-O-methyl ribonucleotide
  modified_base 39: mod_base = OTHER; note = 2'-O-methyl ribonucleotide
  modified_base 40: mod_base = OTHER; note = 2'-O-methyl ribonucleotide
  modified_base 41: mod_base = OTHER; note = 2'-O-methyl ribonucleotide
  modified_base 1..3: mod_base = OTHER; note = phosphorothioate linkage
  modified_base 19..23: mod_base = OTHER; note = phosphorothioate linkage
  modified_base 39..41: mod_base = OTHER; note = phosphorothioate linkage
  misc_feature 1..19: note = RNA
  misc_feature 20..22: note = DNA
  misc_feature 23..41: note = RNA

SEQ ID NO: 93 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(docosyloxy)-5-(hydroxymethyl)tetra hydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 94 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = ribo-2'-propargyl nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 95 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 96 - RNA, 19 nt, synthetic construct
  source 1..19: mol_type = other RNA
  modified_base 1: mod_base = OTHER; note = (2R,3S,4R,5R)-3-(hexadecyloxy)-5-(hydroxymethyl)tet rahydrofuran-2,4-diol nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 97 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = (2R,3S,4R,5R)-3-(hexadecyloxy)-5-(hydroxymethyl)tet rahydrofuran-2,4-diol nucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 98 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = (2R,3S,4R,5R)-3-(hexadecyloxy)-5-(hydroxymethyl)tet rahydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 99 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = (2R,3S,4R,5R)-3-(hexadecyloxy)-5-(hydroxymethyl)tet rahydrofuran-2,4-diol nucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 100 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = (2R,3S,4R,5R)-3-(hexadecyloxy)-5-(hydroxymethyl)tet rahydrofuran-2,4-diol nucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 101 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = (2R,3S,4R,5R)-3-(hexadecyloxy)-5-(hydroxymethyl)tet rahydrofuran-2,4-diol nucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 102 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = (2R,3S,4R,5R)-3-(hexadecyloxy)-5-(hydroxymethyl)tet rahydrofuran-2,4-diol nucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 103 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = (2R,3S,4R,5R)-3-(hexadecyloxy)-5-(hydroxymethyl)tet rahydrofuran-2,4-diol nucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 104 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = (2R,3S,4R,5R)-3-(hexadecyloxy)-5-(hydroxymethyl)tet rahydrofuran-2,4-diol nucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 105 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = (2R,3S,4R,5R)-3-(hexadecyloxy)-5-(hydroxymethyl)tet rahydrofuran-2,4-diol nucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 106 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = (2R,3S,4R,5R)-3-(hexadecyloxy)-5-(hydroxymethyl)tet rahydrofuran-2,4-diol nucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 107 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = (2R,3S,4R,5R)-3-(hexadecyloxy)-5-(hydroxymethyl)tet rahydrofuran-2,4-diol nucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 108 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = (2R,3S,4R,5R)-3-(hexadecyloxy)-5-(hydroxymethyl)tet rahydrofuran-2,4-diol nucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 109 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = (2R,3S,4R,5R)-3-(hexadecyloxy)-5-(hydroxymethyl)tet rahydrofuran-2,4-diol nucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 110 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = (2R,3S,4R,5R)-3-(hexadecyloxy)-5-(hydroxymethyl)tet rahydrofuran-2,4-diol nucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 111 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = (2R,3S,4R,5R)-3-(hexadecyloxy)-5-(hydroxymethyl)tet rahydrofuran-2,4-diol nucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 112 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = (2R,3S,4R,5R)-3-(hexadecyloxy)-5-(hydroxymethyl)tet rahydrofuran-2,4-diol nucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 113 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = (2R,3S,4R,5R)-3-(hexadecyloxy)-5-(hydroxymethyl)tet rahydrofuran-2,4-diol nucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 114 - RNA, 19 nt, synthetic construct
  source 1..19: mol_type = other RNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 115 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 116 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = (2R,3S,4R,5R)-3-(docosyloxy)-5-(hydroxymethyl)tetra hydrofuran-2,4-diol nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 117 - RNA, 19 nt, synthetic construct
  source 1..19: mol_type = other RNA
  modified_base 1: mod_base = OTHER; note = 6-aminohexyl (((2R,3R,4R,5S)-4-(hexadecyloxy)-3,5-d ihydroxytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccctagaactgtcttcgaa

SEQ ID NO: 118 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 2..3: mod_base = OTHER; note = phosphorothioate linkage
  modified_base 17..19: mod_base = OTHER; note = phosphorothioate linkage
ccctagaactgtcttcgaa

SEQ ID NO: 119 - RNA, 18 nt, synthetic construct
  source 1..18: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-fluoro ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 1..2: mod_base = OTHER; note = phosphorothioate linkage
  modified_base 16..18: mod_base = OTHER; note = phosphorothioate linkage
cctagaactgtcttcgaa

SEQ ID NO: 120 - RNA, 19 nt, synthetic construct
  source 1..19: mol_type = other RNA
  modified_base 1: mod_base = OTHER; note = 6-aminohexyl (((2R,3R,4R,5S)-4-(hexadecyloxy)-3,5-d ihydroxytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 2..3: mod_base = OTHER; note = phosphorothioate linkage
  modified_base 17..19: mod_base = OTHER; note = phosphorothioate linkage
ccctagaactgtcttcgaa

SEQ ID NO: 121 - RNA, 20 nt, synthetic construct
  source 1..20: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 1..3: mod_base = OTHER; note = phosphorothioate linkage
  modified_base 18..20: mod_base = OTHER; note = phosphorothioate linkage
accctagaactgtcttcgaa

SEQ ID NO: 122 - RNA, 20 nt, synthetic construct
  source 1..20: mol_type = other RNA
  modified_base 1: mod_base = OTHER; note = 6-aminohexyl (((2R,3R,4R,5S)-4-(hexadecyloxy)-3,5-d ihydroxytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 1..3: mod_base = OTHER; note = phosphorothioate linkage
  modified_base 18..20: mod_base = OTHER; note = phosphorothioate linkage
accctagaactgtcttcgaa

SEQ ID NO: 123 - RNA, 20 nt, synthetic construct
  source 1..20: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 2..4: mod_base = OTHER; note = phosphorothioate linkage
  modified_base 18..20: mod_base = OTHER; note = phosphorothioate linkage
accctagaactgtcttcgaa

SEQ ID NO: 124 - RNA, 20 nt, synthetic construct
  source 1..20: mol_type = other RNA
  modified_base 1: mod_base = OTHER; note = 6-aminohexyl (((2R,3R,4R,5S)-4-(hexadecyloxy)-3,5-d ihydroxytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 2..4: mod_base = OTHER; note = phosphorothioate linkage
  modified_base 18..20: mod_base = OTHER; note = phosphorothioate linkage
accctagaactgtcttcgaa

SEQ ID NO: 125 - RNA, 20 nt, synthetic construct
  source 1..20: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 1..3: mod_base = OTHER; note = phosphorothioate linkage
  modified_base 18..20: mod_base = OTHER; note = phosphorothioate linkage
tccctagaactgtcttcgaa

SEQ ID NO: 126 - RNA, 20 nt, synthetic construct
  source 1..20: mol_type = other RNA
  modified_base 1: mod_base = OTHER; note = 6-aminohexyl (((2R,3R,4R,5S)-4-(hexadecyloxy)-3,5-d ihydroxytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 1..3: mod_base = OTHER; note = phosphorothioate linkage
  modified_base 18..20: mod_base = OTHER; note = phosphorothioate linkage
tccctagaactgtcttcgaa

SEQ ID NO: 127 - RNA, 20 nt, synthetic construct
  source 1..20: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 2..4: mod_base = OTHER; note = phosphorothioate linkage
  modified_base 18..20: mod_base = OTHER; note = phosphorothioate linkage
tccctagaactgtcttcgaa

SEQ ID NO: 128 - RNA, 20 nt, synthetic construct
  source 1..20: mol_type = other RNA
  modified_base 1: mod_base = OTHER; note = 6-aminohexyl (((2R,3R,4R,5S)-4-(hexadecyloxy)-3,5-d ihydroxytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 2..4: mod_base = OTHER; note = phosphorothioate linkage
  modified_base 18..20: mod_base = OTHER; note = phosphorothioate linkage
tccctagaactgtcttcgaa

SEQ ID NO: 129 - DNA, 22 bp, synthetic construct
  source 1..22: mol_type = other DNA
  modified_base 1: mod_base = OTHER; note = 2'-O-methyl ribonucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 1..3: mod_base = OTHER; note = phosphorothioate linkage
  modified_base 18..20: mod_base = OTHER; note = phosphorothioate linkage
  misc_feature 1..20: note = RNA
  misc_feature 21..22: note = DNA
cccctagaactgtcttcgaatt

SEQ ID NO: 130 - DNA, 22 bp, synthetic construct
  source 1..22: mol_type = other DNA
  modified_base 1: mod_base = OTHER; note = 6-aminohexyl (((2R,3S,5R)-3,5-dihydroxytetrahydrofuran-2-yl)methyl) hydrogen phosphate nucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-fluoro ribonucleotide
  modified_base 11: mod_base = OTHER; note = 2'-fluoro ribonucleotide
  modified_base 12: mod_base = OTHER; note = 2'-fluoro ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 22: mod_base = OTHER; note = 2'-O-methyl ribonucleotide
  modified_base 3..5: mod_base = OTHER; note = phosphorothioate linkage
  modified_base 20..22: mod_base = OTHER; note = phosphorothioate linkage
  misc_feature 1..2: note = DNA
  misc_feature 3..22: note = RNA
ttcccctagaactgtcttcgaa

SEQ ID NO: 131 - DNA, 23 bp, synthetic construct
  source 1..23: mol_type = other DNA
  modified_base 1: mod_base = OTHER; note = 6-aminohexyl (((2R,3R,4R,5S)-4-(hexadecyloxy)-3,5-d ihydroxytetrahydrofuran-2-yl)methyl) hydrogen phosphate nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-O-methyl ribonucleotide
  modified_base 11: mod_base = OTHER; note = 2'-fluoro ribonucleotide
  modified_base 12: mod_base = OTHER; note = 2'-fluoro ribonucleotide
  modified_base 13: mod_base = OTHER; note = 2'-fluoro ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 22: mod_base = OTHER; note = 2'-O-methyl ribonucleotide
  modified_base 23: mod_base = OTHER; note = 2'-O-methyl ribonucleotide
  modified_base 4..6: mod_base = OTHER; note = phosphorothioate linkage
  modified_base 21..23: mod_base = OTHER; note = phosphorothioate linkage
  misc_feature 1: note = RNA
  misc_feature 2..3: note = DNA
  misc_feature 4..23: note = RNA
tttcccctagaactgtcttcgaa

SEQ ID NO: 132 - DNA, 23 bp, synthetic construct
  source 1..23: mol_type = other DNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 23: mod_base = OTHER; note = (2R,3S,4R,5R)-3-(hexadecyloxy)-5-(hydroxymethyl)tet rahydrofuran-2,4-diol nucleotide
  modified_base 1..3: mod_base = OTHER; note = phosphorothioate linkage
  modified_base 18..20: mod_base = OTHER; note = phosphorothioate linkage
  misc_feature 1..20: note = RNA
  misc_feature 21..22: note = DNA
  misc_feature 23: note = RNA
cccctagaactgtcttcgaattt

SEQ ID NO: 133 - RNA, 19 nt, synthetic construct
  source 1..19: mol_type = other RNA
  modified_base 1: mod_base = OTHER; note = 6-aminohexyl (((2R,3R,4S,5R)-3,5-dihydroxy-4-methox ytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-fluoro ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-O-methyl ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 1..3: mod_base = OTHER; note = phosphorothioate linkage
  modified_base 17..19: mod_base = OTHER; note = phosphorothioate linkage
ccgacattcctcggtatta

SEQ ID NO: 134 - RNA, 20 nt, synthetic construct
  source 1..20: mol_type = other RNA
  modified_base 1: mod_base = OTHER; note = 6-aminohexyl (((2R,3R,4S,5R)-4-(docosyloxy)-3,5-dih ydroxytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 2..4: mod_base = OTHER; note = phosphorothioate linkage
  modified_base 18..20: mod_base = OTHER; note = phosphorothioate linkage
accgacattcctcggtatta

SEQ ID NO: 135 - RNA, 20 nt, synthetic construct
  source 1..20: mol_type = other RNA
  modified_base 1: mod_base = OTHER; note = 6-aminohexyl (((2R,3R,4S,5R)-4-(docosyloxy)-3,5-dih ydroxytetrahydrofuran-2-yl)methyl) hydrogen phosphate nucleotide
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 2..4: mod_base = OTHER; note = phosphorothioate linkage
  modified_base 18..20: mod_base = OTHER; note = phosphorothioate linkage
tccgacattcctcggtatta

SEQ ID NO: 136 - DNA, 22 bp, synthetic construct
  source 1..22: mol_type = other DNA
  modified_base 1: mod_base = OTHER; note = 6-aminohexyl (((2R,3R,4S,5R)-4-(docosyloxy)-3,5-dih ydroxytetrahydrofuran-2-yl)methyl) hydrogen phosphate nucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-O-methyl ribonucleotide
  modified_base 9: mod_base = OTHER; note = 2'-O-methyl ribonucleotide
  modified_base 10: mod_base = OTHER; note = 2'-fluoro ribonucleotide
  modified_base 11: mod_base = OTHER; note = 2'-fluoro ribonucleotide
  modified_base 12: mod_base = OTHER; note = 2'-fluoro ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 21: mod_base = OTHER; note = 2'-O-methyl ribonucleotide
  modified_base 22: mod_base = OTHER; note = 2'-O-methyl ribonucleotide
  modified_base 4..6: mod_base = OTHER; note = phosphorothioate linkage
  modified_base 20..22: mod_base = OTHER; note = phosphorothioate linkage
  misc_feature 1: note = RNA
  misc_feature 2..3: note = DNA
  misc_feature 4..22: note = RNA
tttccgacattcctcggtatta

SEQ ID NO: 137 - RNA, 20 nt, synthetic construct
  source 1..20: mol_type = other RNA
  modified_base 1: mod_base = OTHER; note = C18 5-U Phosphoramidite modified with 6-aminohexyl (((2R,3R,4S,5R)-3,5-dihydroxy-4-methoxytetrahydrofuran-2-y l)methyl) hydrogen phosphate
  modified_base 2: mod_base = OTHER; note = 2'-O-methyl ribonucleotide
  modified_base 3: mod_base = OTHER; note = 2'-O-methyl ribonucleotide
  modified_base 4: mod_base = OTHER; note = 2'-O-methyl ribonucleotide
  modified_base 5: mod_base = OTHER; note = 2'-O-methyl ribonucleotide
  modified_base 6: mod_base = OTHER; note = 2'-O-methyl ribonucleotide
  modified_base 7: mod_base = OTHER; note = 2'-O-methyl ribonucleotide
  modified_base 8: mod_base = OTHER; note = 2'-fluoro ribonucleotide
  modified_base 9: mod_base = OTHER; note = 2'-fluoro ribonucleotide
  modified_base 10: mod_base = OTHER; note = 2'-fluoro ribonucleotide
  modified_base 11: mod_base = OTHER; note = 2'-O-methyl ribonucleotide
  modified_base 12: mod_base = OTHER; note = 2'-O-methyl ribonucleotide
  modified_base 13: mod_base = OTHER; note = 2'-O-methyl ribonucleotide
  modified_base 14: mod_base = OTHER; note = 2'-O-methyl ribonucleotide
  modified_base 15: mod_base = OTHER; note = 2'-O-methyl ribonucleotide
  modified_base 16: mod_base = OTHER; note = 2'-O-methyl ribonucleotide
  modified_base 17: mod_base = OTHER; note = 2'-O-methyl ribonucleotide
  modified_base 18: mod_base = OTHER; note = 2'-O-methyl ribonucleotide
  modified_base 19: mod_base = OTHER; note = 2'-O-methyl ribonucleotide
  modified_base 20: mod_base = OTHER; note = 2'-O-methyl ribonucleotide
  modified_base 2..4: mod_base = OTHER; note = phosphorothioate linkage
  modified_base 18..20: mod_base = OTHER; note = phosphorothioate linkage
tccgacattcctcggtatta

(19) United States
(12) Patent Application Publication
(10) Pub. No.: US 2026/0216357 A1
(43) Pub. Date: Jul. 30, 2026

(54) LIPID ANTIBODY OLIGONUCLEOTIDE CONJUGATES COMPOSITIONS AND USES THEREOF

(71) Applicant: Atrium Therapeutics, Inc., San Diego, CA (US)

(72) Inventors: Venkata Ramana DOPPALAPUDI, San Diego, CA (US); Michael Caramian COCHRAN, Palo Alto, CA (US); Subbarao NALLAGATLA, San Diego, CA (US); Tyler ALBIN, Encinitas, CA (US); Isaac MARKS, San Diego, CA (US); Varun GOEL, Carlsbad, CA (US); Marc Andrew HARTMANN, San Diego, CA (US)

(21) Appl. No.: 19/370,553

(22) Filed: Oct. 27, 2025

Related U.S. Application Data
(60) Provisional application No. 63/712,997, filed on Oct. 28, 2024.

(51) Int. Cl. A61K 47/68; A61K 47/54; A61P 21/00

(52) U.S. Cl. CPC A61K 47/6807; A61K 47/543; A61K 47/6849; A61K 47/6889; A61P 21/00

(57) ABSTRACT

Disclosed herein are compositions and pharmaceutical formulations that comprise a binding moiety conjugated to a polynucleic acid molecule linked to a lipophilic moiety. Also described herein include methods for treating a disease or disorder which utilize a composition or a pharmaceutical formulation comprising a binding moiety conjugated to a polynucleic acid molecule linked to a lipophilic moiety.

### CROSS REFERENCE

This application claims the benefit of U.S. Provisional Application No. 63/712,997 filed Oct. 28, 2024, which is incorporated herein by reference in its entirety.

### REFERENCE TO A SEQUENCE LISTING XML

This application contains a Sequence Listing which has been submitted electronically in XML format. The Sequence Listing XML is incorporated herein by reference. Said XML file, created on Jan. 2, 2026, is named 45532-790_201_SL.xml and is 1,733,510 bytes in size.

### BACKGROUND OF THE DISCLOSURE

Gene suppression by RNA-induced gene silencing provides several levels of control: transcription inactivation, small interfering RNA (siRNA)-induced mRNA degradation, and siRNA-induced transcriptional attenuation. In some instances, RNA interference (RNAi) provides long lasting effect over multiple cell divisions. As such, RNAi represents a viable method useful for drug target validation, gene function analysis, pathway analysis, and disease therapeutics. Although the in vivo delivery of RNAi therapeutics in the liver has been successful, the delivery of RNAi agent to other tissues beside liver remains difficult. Therefore, there is a need for new methods for efficient delivery of siRNA in tissues other than the liver.

### SUMMARY OF THE DISCLOSURE

Disclosed herein is a polynucleotide conjugate comprising a binding moiety conjugated to a polynucleotide molecule. In some embodiments, the polynucleotide conjugate further comprises a lipophilic moiety. In some embodiments, the lipophilic moiety is a linear or branched alkyl group. In some embodiments, the linear alkyl group is selected from the group consisting of C6, C8, C10, C12, C14, C16, C18, C20, C22, C24 and C26 hydrocarbon chain. In some embodiments, the linear alkyl group is a C16 or C22 hydrocarbon chain. In some embodiments, the lipophilic moiety is conjugated to a nucleotide of the polynucleotide molecule. In some embodiments, the lipophilic moiety is conjugated to a sugar moiety of the nucleotide of the polynucleotide molecule at the 2′- or 3′-carbon or a modification made thereof. In some embodiments, the polynucleotide molecule is a single-stranded or double-stranded polynucleotide. In some embodiments, the double-stranded polynucleotide is an siRNA comprising a guide strand and a passenger strand. In some embodiments, the double-stranded polynucleotide comprises a passenger strand comprising a PMO molecule and a guide strand comprising an RNA molecule, forming a heteroduplex structure. In some embodiments, the lipophilic moiety is conjugated to the passenger strand. In some embodiments, the lipophilic moiety is conjugated to a nucleotide of the passenger strand at one of the positions 2-8 from the 5′ end. In some embodiments, the lipophilic moiety is conjugated to a nucleotide of the passenger strand at position 2, 3, or 6 from the 5′ end. In some embodiments, the lipophilic moiety is conjugated to the guide strand. In some embodiments, 16-21 from the 5′ end. In some embodiments, the lipophilic moiety is conjugated to the nucleotide via a first linker. In some embodiments, the first linker is selected from the compound of Formula (A) or Formula (B):

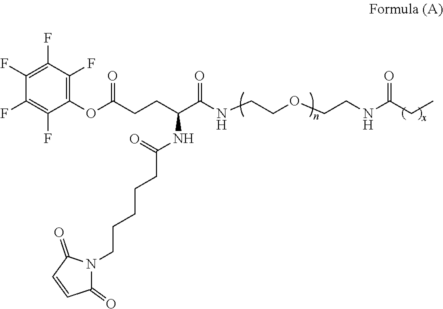

wherein n is 1-20; and x is 1-30; or

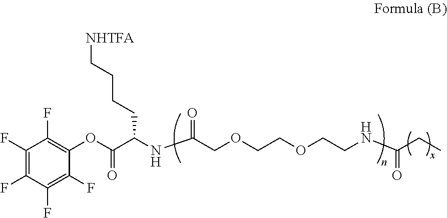

wherein n is 1-10; and x is 1-30.

In some embodiments, the first linker is selected from the compounds listed in Table 1 and Table 2. In some embodiments, the lipophilic moiety is further modified with a triazole (tz), an oleyl, a carboxylic acid, or a combination thereof. In some embodiments, the lipophilic moiety further modified with tzC14, tzC16, tzC18, tzC20, tzC18-oleyl, or 2′-O—C16. In some embodiments, the lipophilic moiety is further modified with a triazole (tz) by click chemistry. In some embodiments, the polynucleotide molecule comprises a blunt end, an unmatched end or an overhang. In some embodiments, the polynucleotide molecule comprises a blunt end, an unmatched end or an overhang at 5′ terminus, 3′ terminus, or both at the guide strand. In some embodiments, the overhang is stable or degradable by a nuclease. In some embodiments, the lipophilic moiety is conjugated to a second linker coupling the binding moiety to the polynucleotide molecule. In some embodiments, the second linker is a cleavable linker or a non-cleavable linker. In some embodiments, the second linker is a bond, C1-C30 alkyl group, a homobifunctional linker or a heterobifunctional linker, optionally conjugated to a C1-C6 alkyl group. In some embodiments, the second linker is a Bismal linker, SMCC linker, MBS linker, C16 linker, or C22 linker. In some embodiments, the lipophilic moiety is conjugated to the binding moiety. In some embodiments, the binding moiety is selected from the group consisting of peptides, toxins, sugars, carbohydrates, and polymers. In some embodiments, the binding moiety is selected from the group consisting of hormones, steroids, phospholipids, di- and triacylglycerols, fatty acids, hydrocarbons, enzyme substrates, biotin, digoxigenin, and polysaccharides. In some embodiments, the peptide is a bicyclic peptide or a tricyclic peptide. In some embodiments, the binding moiety is an antibody or antigen binding fragment thereof. In some embodiments, the antibody or antigen binding fragment thereof comprises a humanized antibody or antigen binding fragment thereof, chimeric antibody or antigen binding fragment thereof, monoclonal antibody or antigen binding fragment thereof, single-arm antibody, Fab-Fc fusion antibody, VHH-Fc fusion antibody, monovalent Fab, monovalent Fab′, divalent Fab2, single-chain variable fragment (scFv), diabody, minibody, nanobody, single-domain antibody (sdAb), VHH, or camelid antibody or antigen binding fragment thereof. In some embodiments, the antibody is a monoclonal antibody. In some embodiments, the antigen binding fragment is a Fab or Fab′. In some embodiments, the binding moiety is capable of binding to a cell surface receptor. In some embodiments, the cell surface receptor is a transferrin receptor (TfR). In some embodiments, the transferrin receptor is transferrin receptor I. In some embodiments, the polynucleotide molecule comprises at least one 2′ modified nucleotide, at least one modified internucleotide linkage, or at least one inverted abasic moiety. In some embodiments, the at least one 2′ modified nucleotide comprises 2′-O-methyl, 2′-O-methoxyethyl (2′-O-MOE), 2′-O-aminopropyl, 2′-deoxy, 2-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In some embodiments, the at least one 2′ modified nucleotide comprises locked nucleic acid (LNA) or ethylene nucleic acid (ENA). In some embodiments, the at least one modified internucleotide linkage comprises a phosphorothioate linkage or a phosphorodithioate linkage, or a phosphoryl guanidine (PG) linkage. In some embodiments, the polynucleotide molecule further comprises a 5′ terminal vinylphosphonate modified nucleotide. In some embodiments, the polynucleotide conjugate has a drug antibody ratio (DAR) of 1, 2, 3, 4, 5, or more. In some embodiments, the lipophilic moiety comprises a hydrocarbon with more than 16 carbons when DAR is 1, 2, 3, 4, or 5. In some embodiments, the hydrocarbon with more than 16 carbons is selected from C17, C18, C19, C20, C21, C22, C23, and C24 hydrocarbon chain. In some embodiments, the lipophilic moiety comprises a hydrocarbon chain with 16 or less carbons when DAR is 3 or more. In some embodiments, the hydrocarbon chain with 16 or less carbons is selected from C8, C9, C10, C11, C12, C13, C14, and C15 hydrocarbon chain. In some embodiments, the lipophilic moiety comprises a hydrocarbon chain with 16 or 22 carbons when DAR is 1, 2, 3, 4, 5 or more. In some embodiments, the polynucleotide conjugate comprises two or more lipophilic moieties. In some embodiments, the two or more lipophilic moieties are the same type of lipophilic moiety. In some embodiments, the two or more lipophilic moieties are different types of lipophilic moieties. In some embodiments, the polynucleotide molecule comprises a first polynucleotide and a second polynucleotide, and the first and second polynucleotides hybridizes different target sequences. In some embodiments, the first and second polynucleotides target mRNAs of different genes.

Also provided, in some aspects, is a method of delivering a polynucleotide molecule to a tissue of a subject comprising administering the polynucleotide conjugate disclosed herein. Also provided, in some aspects, is a method of increasing the stability of a polynucleotide molecule in a tissue of a subject comprising administering the polynucleotide conjugate disclosed herein. In some embodiments, the polynucleotide conjugate is administered systemically. In some embodiments, the polynucleotide conjugate is administered subcutaneously or intravenously. In some embodiments, the tissue is an extrahepatic tissue. In some embodiments, the tissue is a muscle tissue. In some embodiments, the muscle tissue is a skeletal muscle tissue or a cardiac muscle tissue. In some embodiments, the higher amount of a polynucleotide molecule is maintained within the tissue compared to a polynucleotide conjugate without a lipophilic moiety at least 60, 90, or 120 days after administration.

Also provided, in some aspects, is a method of modulating mRNA expression levels in a tissue of a subject comprising administering the polynucleotide conjugate disclosed herein. Also provided, in some aspects, is a method of treating muscle atrophy or myotonic dystrophy in a subject in need thereof comprising administering the polynucleotide conjugate disclosed herein. In some embodiments, the polynucleotide conjugate is administered systemically. In some embodiments, the polynucleotide conjugate is administered subcutaneously or intravenously. In some embodiments, the polynucleotide conjugate is delivered to a muscle tissue. In some embodiments, the muscle tissue is a skeletal muscle tissue or a cardiac muscle tissue. In some embodiments, the polynucleotide conjugate mediates RNA interference against a target mRNA in the muscle tissue of the subject. In some embodiments, the polynucleotide conjugate decreases mRNA expression levels of a target gene in the muscle tissue of the subject. In some embodiments, the polynucleotide conjugate decreases mRNA expression levels of the target gene by 20%, 30%, 40%, 50%, 60%, 70%, 80%, or more in the muscle tissue of the subject. In some embodiments, the polynucleotide conjugate decreases mRNA expression levels of the target gene for 2 days, 28 days, 84 days or longer. In some embodiments, the polynucleotide conjugate hybridizes to at least 8 contiguous bases of the target gene in the muscle tissue of the subject. In some embodiments, the target gene is selected from DMPK, DMD, DUX4, GYS1, PLN, PRKAG2, CNBP, MSTN, or SSB. In some embodiments, the myotonic dystrophy is myotonic dystrophy type 1 (DM1).

Also disclosed herein is a pharmaceutical composition comprising the polynucleotide conjugate disclosed herein and a pharmaceutically acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C are representative plots showing siRNA tissue concentrations or MSTN mRNA expression levels in gastric or heart tissues obtained from mice during a time course of 84 days (28, 56 or 84 days) after a single intravenous injection of the lipid AOC (mTfR1-bismal-pos.6.C16.siMSTN DAR1) at a dose of 0.75 mg/kg of siRNA.

FIG. 1A is a representative plot showing siRNA tissue concentrations in gastrocnemius (gastroc) tissue obtained from mice during a time course of 84 days (28, 56 or 84 days) after a single intravenous injection of the lipid AOC (mTfR1-bismal-pos.6.C16.siMSTN DAR1) at a dose of 0.75 mg/kg of siRNA.

FIG. 1B is a representative plot showing siRNA tissue concentrations in heart tissue obtained from mice during a time course of 84 days (28, 56 or 84 days) after a single intravenous injection of the lipid AOC (mTfR1-bismal-pos.6.C16.siMSTN DAR1) at a dose of 0.75 mg/kg of siRNA.

FIG. 1C is a representative plot showing MSTN mRNA expression levels in gastrocnemius (gastroc) tissue obtained from mice during a time course of 84 days (28, 56 or 84 days) after a single intravenous injection of the lipid AOC (mTfR1-bismal-pos.6.C16.siMSTN DAR1) at a dose of 0.75 mg/kg of siRNA.

FIG. 2 illustrates plots illustrating binding affinity analysis of DMPK AOC (mTfR1-mAb-siDMPK DAR1), DMPK lipid AOC conjugated with 2′-O—C16 at position 6 of the passenger strand (mTfR1-mAb-pos6.C16.siDMPK DAR1), or isotype control mAb AOCs (mIsotype-mAb-siDMPK DAR1, mIsotype-mAb-pos6.C16.siDMPK DAR1).

FIGS. 3A-3D are plots showing siRNA tissue concentrations in gastrocnemius (gastroc), tibialis anterior (TA), heart, or liver tissue obtained from mice during a time course of 84 days (14, 28, 56 or 84 days) after a single intravenous injection of AOC (mTfR1-mAb-siDMPK DAR1), a lipid AOC (mTfR1-mAb-pos6.C16.siDMPK DAR1), or isotype control mAb AOCs (mIsotype-mAb-siDMPK DAR1, mIsotype-mAb-pos6.C16.siDMPK DAR1) at a dose of 0.75 mg/kg of siRNA.

FIG. 3A is a plot showing siRNA tissue concentrations in gastrocnemius (gastroc) tissue obtained from mice during a time course of 84 days (14, 28, 56 or 84 days) after a single intravenous injection of the unmodified AOC (mTfR1-mAb-siDMPK DAR1), lipid AOC (mTfR1-mAb-pos6.C16.siDMPK DAR1), or untargeted isotype control mAb AOCs (mIsotype-mAb-siDMPK DAR1, mIsotype-mAb-pos6.C16.siDMPK DAR1) at a dose of 0.75 mg/kg of siRNA.

FIG. 3B is a plot showing siRNA tissue concentrations in tibialis anterior (TA) tissue obtained from mice during a time course of 84 days (14, 28, 56 or 84 days) after a single intravenous injection of the unmodified AOC (mTfR1-mAb-siDMPK DAR1), lipid AOC (mTfR1-mAb-pos6.C16.siDMPK DAR1), or untargeted isotype control mAb AOCs (mIsotype-mAb-siDMPK DAR1, mIsotype-mAb-pos6.C16.siDMPK DAR1) at a dose of 0.75 mg/kg of siRNA.

FIG. 3C is a plot showing siRNA tissue concentrations in heart muscle tissue obtained from mice during a time course of 84 days (14, 28, 56 or 84 days) after a single intravenous injection of the unmodified AOC (mTfR1-mAb-siDMPK DAR1) or lipid AOC (mTfR1-mAb-pos6.C16.siDMPK DAR1), or untargeted isotype control mAb AOCs (mIsotype-mAb-siDMPK DAR1, mIsotype-mAb-pos6.C16.siDMPK DAR1) at a dose of 0.75 mg/kg of siRNA.

FIG. 3D is a plot showing siRNA tissue concentrations in liver tissue obtained from mice during a time course of 84 days (14, 28, 56 or 84 days) after a single intravenous injection of the unmodified AOC (mTfR1-mAb-siDMPK DAR1) or lipid AOC (mTfR1-mAb-pos6.C16.siDMPK DAR1), or untargeted isotype control mAb AOCs (mIsotype-mAb-siDMPK DAR1, mIsotype-mAb-pos6.C16.siDMPK DAR1) at a dose of 0.75 mg/kg of siRNA.

FIGS. 4A-4C are plots showing DMPK mRNA expression levels in gastrocnemius (gastroc), tibialis anterior (TA), and heart muscle tissue obtained from mice during a time course of 84 days (14, 28, 56 or 84 days) after a single intravenous injection of the unmodified AOC (mTfR1-mAb-siDMPK DAR1), lipid AOC (mTfR1-mAb-pos6.C16.siDMPK DAR1), or untargeted isotype control mAb AOCs (mIsotype-mAb-siDMPK DAR1, mIsotype-mAb-pos6.C16.siDMPK DAR1) at a dose of 0.75 mg/kg of siRNA.

FIG. 4A are plots showing DMPK mRNA expression levels in gastrocnemius (gastroc) obtained from mice during a time course of 84 days (14, 28, 56 or 84 days) after a single intravenous injection of the unmodified AOC (mTfR1-mAb-siDMPK DAR1) or lipid AOC (mTfR1-mAb-pos6.C16.siDMPK DAR1), or untargeted isotype control mAb AOCs (mIsotype-mAb-siDMPK DAR1, mIsotype-mAb-pos6.C16.siDMPK DAR1) at a dose of 0.75 mg/kg of siRNA.

FIG. 4B are plots showing DMPK mRNA expression levels in tibialis anterior (TA) obtained from mice during a time course of 84 days (14, 28, 56 or 84 days) after a single intravenous injection of the unmodified AOC (mTfR1-mAb-siDMPK DAR1) or lipid AOC (mTfR1-mAb-pos6.C16.siDMPK DAR1) or untargeted isotype control mAb AOCs (mIsotype-mAb-siDMPK DAR1, mIsotype-mAb-pos6.C16.siDMPK DAR1) at a dose of 0.75 mg/kg of siRNA.

FIG. 4C are plots showing DMPK mRNA expression levels in heart muscle tissue obtained from mice during a time course of 84 days (14, 28, 56 or 84 days) after a single intravenous injection of the unmodified AOC (mTfR1-mTfR1-mAb-siDMPK DAR1), lipid AOC (mTfR1-mTfR1-mAb-pos6.C16.siDMPK DAR1), or untargeted isotype control mAb AOCs (mIsotype-mAb-siDMPK DAR1, mIsotype-mAb-pos6.C16.siDMPK DAR1) at a dose of 0.75 mg/kg of siRNA

FIG. 5 is a plot showing binding assay of mAb-siRNA DAR1 lipid conjugates to HSA as detected by fluorescence polarimetry assay.

FIGS. 6A-6B are bar graphs showing DMPK siRNA tissue concentrations in gastrocnemius (gastroc), tibialis anterior (TA), liver, and heart tissues obtained from mice 28 or 56 days after a single intravenous injection of the control AOC (mTfR1-mAb-DMPK DAR1) or lipid AOC, AOC conjugated with 2′-O—C16 at passenger strand position 3, 6, 17, or 19 (the lipid C16 at different 4 nucleotide positions, pos.3, pos.6, pos.17, pos.19, of the passenger strand) of the DMPK siRNA (mTfR1-mAb-pos.6.C16.siDMPK DAR1, mTfR1-mAb-pos.3.C16.siDMPK DAR1, mTfR1-mAb-pos.17.C16.siDMPK DAR1, or mTfR1-mAb-pos.19.C16.siDMPK DAR1) at a dose of 0.5 mg/kg of siRNA.

FIG. 6A is bar graph showing siRNA tissue concentrations in gastrocnemius (gastroc), tibialis anterior (TA), liver, and heart tissues obtained from mice 28 days after a single intravenous injection of the control AOC (mTfR1-mAb-DMPK DAR1) or AOC conjugated with the lipid C16 at different nucleotide positions (pos.3, pos.6, pos.17, pos.19) of the passenger strand of the DMPK siRNA (mTfR11-mTfR1-mAb-pos.6.C16.siDMPK DAR1, mTfR1-mTfR1-mAb-pos.3.C16.siDMPK DAR1, mTfR1-mTfR1-mAb-pos.17.C16.siDMPK DAR1, or mTfR1-mTfR1-mAb-pos.19.C16.siDMPK DAR1) at a dose of 0.5 mg/kg of siRNA.

FIG. 6B is bar graph showing siRNA tissue concentrations in gastrocnemius (gastroc), tibialis anterior (TA), liver, and heart tissue obtained from mice 56 days after a single intravenous injection of the control AOC (mTfR1-mAb-DMPK DAR1) or AOC conjugated with the lipid C16 at different nucleotide positions (pos.3, pos.6, pos.17, pos.19) of the passenger strand of the DMPK siRNA (mTfR1-mTfR1-mAb-pos.6.C16.siDMPK DAR1, mTfR1-mTfR1-mAb-pos.3.C16.siDMPK DAR1, mTfR1-mTfR1-mAb-pos.17.C16.siDMPK DAR1, or mTfR1-mTfR1-mAb-pos.19.C16.siDMPK DAR1) at a dose of 0.5 mg/kg of siRNA.

FIGS. 7A-7B are bar graphs showing SSB mRNA expression levels in gastrocnemius (gastroc), tibialis anterior (TA), liver, or heart obtained from mice 28 or 56 days after a single intravenous injection of the control AOC (mTfR1-mAb-siSSB DAR1) or AOC modified with the lipid C16 at different nucleotide positions (pos.3, pos.6, pos.17, pos.19) of the passenger strand of the SSB siRNA (mTfR1-mTfR1-mAb-pos.6.C16.siSSB DAR1, mTfR1-mTfR1-mAb-pos.3.C16.siSSB DAR1, mTfR1-mTfR1-mAb-pos.17.C16.siSSB DAR1, or mTfR1-mTfR1-mAb-pos.19.C16.siSSB DAR1) at a dose of 0.5 mg/kg of siRNA.

FIG. 7A are bar graphs showing SSB mRNA expression levels in gastrocnemius (gastroc), tibialis anterior (TA), liver, or heart tissue obtained from mice 28 days after a single intravenous injection of the control AOC (mTfR1-mAb-siSSB DAR1) or AOC conjugated with the lipid C16 at different nucleotide positions (pos.3, pos.6, pos.17, pos.19) of the passenger strand of the SSB siRNA (mTfR1-mTfR1-mAb-pos.6.C16.siSSB DAR1, mTfR1-mTfR1-mAb-pos.3.C16.siSSB DAR1, mTfR1-mTfR1-mAb-pos.17.C16.siSSB DAR1, or mTfR1-mTfR1-mAb-pos.19.C16.siSSB DAR1) at a dose of 0.5 mg/kg of siRNA.

FIG. 7B are bar graphs showing SSB mRNA expression levels in gastrocnemius (gastroc), tibialis anterior (TA), liver, or heart tissue obtained from mice 56 days after a single intravenous injection of the control AOC (mTfR1-mAb-siSSB DAR1) or AOC conjugated with the lipid C16 at different nucleotide positions (pos.3, pos.6, pos.17, pos.19) of the passenger strand of the SSB siRNA (mTfR1-mTfR1-mAb-pos.6.C16.siSSB DAR1, mTfR1-mTfR1-mAb-pos.3.C16.siSSB DAR1, mTfR1-mTfR1-mAb-pos.17.C16.siSSB DAR1, or mTfR1-mTfR1-mAb-pos.19.C16.siSSB DAR1) at a dose of 0.5 mg/kg of siRNA.

FIGS. 8A-8D are bar graphs showing SSB siRNA tissue concentration in gastrocnemius (gastroc), tibialis anterior (TA), heart, or liver tissue obtained from mice 28 days after a single intravenous injection of the control AOC (mTfR1-mAb-siSSB DAR1) and AOCs with the numerous different lipids attached at position 6 nucleotide of the passenger strand (mTfR1-mTfR1-mAb-pos6.tzC14.siSSB, mTfR1-mAb-pos6.tzC16.siSSB, mTfR1-mAb-pos6.tzC18.siSSB, mTfR1-mAb-pos6.tzC20.siSSB, mTfR1-mAb-pos6.tzC18-oleyl.siSSB, mTfR1-mAb-pos6.tzC18-int.acid.siSSB, mTfR1-mAb-pos6.tzC20da.siSSB, mTfR1-mAb-pos.3.C16.pos6.C16.siSSB, mTfR1-mAb-pos6.C16.siSSB) at a dose of 0.5 mg/kg of siRNA.

FIG. 8A are bar graphs showing SSB siRNA tissue concentration in gastrocnemius (gastroc) tissue obtained from mice 28 days after a single intravenous injection of the control AOC (mTfR1-mAb-siSSB DAR1) and AOCs with the numerous different lipids attached at position 6 nucleotide of the passenger strand (mTfR1-mAb-pos6.tzC14.siSSB, mTfR1-mAb-pos6.tzC16.siSSB, mTfR1-mAb-pos6.tzC18.siSSB, mTfR1-mAb-pos6.tzC20.siSSB, mTfR1-mAb-pos6.tzC18-oleyl.siSSB, mTfR1-mAb-pos6.tzC18-int.acid.siSSB, mTfR1-mAb-pos6.tzC20da.siSSB, mTfR1-mAb-pos.3.C16.pos6.C16.siSSB, mTfR1-mAb-pos6.C16.siSSB) at a dose of 0.5 mg/kg of siRNA.

FIG. 8B are bar graphs showing SSB siRNA tissue concentration in tibialis anterior (TA) tissue obtained from mice 28 days after a single intravenous injection of the control AOC (mTfR1-mAb-siSSB DAR1) and AOCs with the numerous different lipids attached at position 6 nucleotide of the passenger strand (mTfR1-mAb-pos6.tzC14.siSSB, mTfR1-mAb-pos6.tzC16.siSSB, mTfR1-mAb-pos6.tzC18.siSSB, mTfR1-mAb-pos6.tzC20.siSSB, mTfR1-mAb-pos6.tzC18-oleyl.siSSB, mTfR1-mAb-pos6.tzC18-int.acid.siSSB, mTfR1-mAb-pos6.tzC20da.siSSB, mTfR1-mAb-pos.3.C16.pos6.C16.siSSB, mTfR1-mAb-pos6.C16.siSSB) at a dose of 0.5 mg/kg of siRNA.

FIG. 8C are bar graphs showing SSB siRNA tissue concentration heart tissue obtained from mice 28 days after a single intravenous injection of the control AOC (mTfR1-mAb-siSSB DAR1) and AOCs with the numerous different lipids attached at position 6 nucleotide of the passenger strand (mTfR1-mAb-pos6.tzC14.siSSB, mTfR1-mAb-pos6.tzC16.siSSB, mTfR1-mAb-pos6.tzC18.siSSB, mTfR1-mAb-pos6.tzC20.siSSB, mTfR1-mAb-pos6.tzC18-oleyl.siSSB, mTfR1-mAb-pos6.tzC18-int.acid.siSSB, mTfR1-mAb-pos6.tzC20da.siSSB, mTfR1-mAb-pos.3.C16.pos6.C16.siSSB, mTfR1-mAb-pos6.C16.siSSB) at a dose of 0.5 mg/kg of siRNA.

FIG. 8D are bar graphs showing SSB siRNA tissue concentration liver tissue obtained from mice 28 days after a single intravenous injection of the control AOC (mTfR1-mAb-siSSB DAR1) and AOCs with the numerous different lipids attached at position 6 nucleotide of the passenger strand (mTfR1-mAb-pos6.tzC14.siSSB, mTfR1-mAb-pos6.tzC16.siSSB, mTfR1-mAb-pos6.tzC18.siSSB, mTfR1-mAb-pos6.tzC20.siSSB, mTfR1-mAb-pos6.tzC18-oleyl.siSSB, mTfR1-mAb-pos6.tzC18-int.acid.siSSB, mTfR1-mAb-pos6.tzC20da.siSSB, mTfR1-mAb-pos.3.C16.pos6.C16.siSSB, mTfR1-mAb-pos6.C16.siSSB) at a dose of 0.5 mg/kg of siRNA.

FIGS. 9A-9C are bar graphs showing SSB mRNA expression levels in gastrocnemius (gastroc), tibialis anterior (TA), or heart tissue obtained from mice 28 days after a single intravenous injection of the control AOC (mTfR1-mAb-siSSB DAR1) and AOCs conjugated with the lipid C14, C16, C18, or C20 as well as oleyl, and carboxylic acid-modified lipids at position 3 or position 6 (pos.3 or pos.6) with 2′-triazole (“tz”) of the passenger strand of the SSB siRNA (mTfR1-mAb-pos6.tzC14.siSSB, mTfR1-mAb-pos6.tzC16.siSSB, mTfR1-mAb-pos6.tzC18.siSSB, mTfR1-mAb-pos6.tzC20.siSSB, mTfR1-mAb-pos6.tzC18-oleyl.siSSB, mTfR1-mAb-pos6.tzC18-int.acid.siSSB, mTfR1-mAb-pos6.tzC20da.siSSB, mTfR1-mAb-pos.3.C16.pos6.C16.siSSB, mTfR1-mAb-pos6.C16.siSSB)) at a dose of 0.5 mg/kg of siRNA.

FIG. 9A is a bar graph showing SSB mRNA expression levels in gastrocnemius (gastroc) obtained from mice 28 days after a single intravenous injection of the control AOC (mTfR1-mAb-siSSB DAR1) and AOCs conjugated with the lipid C14, C16, C18, or C20 as well as oleyl, and carboxylic acid-modified lipids at position 3 nucleotide or position 6 nucleotide (pos.3 or pos.6) with 2′-triazole (“tz”) of the passenger strand of the SSB siRNA (mTfR1-mAb-pos6.tzC14.siSSB, mTfR1-mAb-pos6.tzC16.siSSB, mTfR1-mAb-pos6.tzC18.siSSB, mTfR1-mAb-pos6.tzC20.siSSB, mTfR1-mAb-pos6.tzC18-oleyl.siSSB, mTfR1-mAb-pos6.tzC18-int.acid.siSSB, mTfR1-mAb-pos6.tzC20da.siSSB, mTfR1-mAb-pos.3.C16.pos6.C16.siSSB, mTfR1-mAb-pos6.C16.siSSB) at a dose of 0.5 mg/kg of siRNA.

FIG. 9B is a bar graph showing SSB mRNA expression levels in tibialis anterior (TA) obtained from mice 28 days after a single intravenous injection of the control AOC (mTfR1-mAb-siSSB DAR1) and AOCs conjugated with the lipid C14, C16, C18, or C20 as well as oleyl, and carboxylic acid-modified lipids at position 3 nucleotide or position 6 nucleotide (pos.3 or pos.6) with 2′-triazole (“tz”) of the passenger strand of the SSB siRNA (mTfR1-mAb-pos6.tzC14.siSSB, mTfR1-mAb-pos6.tzC16.siSSB, mTfR1-mAb-pos6.tzC18.siSSB, mTfR1-mAb-pos6.tzC20.siSSB, mTfR1-mAb-pos6.tzC18-oleyl.siSSB, mTfR1-mAb-pos6.tzC18-int.acid.siSSB, mTfR1-mAb-pos6.tzC20da.siSSB, mTfR1-mAb-pos.3.C16.pos6.C16.siSSB, mTfR1-mAb-pos6.C16.siSSB) at a dose of 0.5 mg/kg of siRNA.

FIG. 9C is a bar graph showing SSB mRNA expression levels in heart obtained from mice 28 days after a single intravenous injection of the control AOC (mTfR1-mAb-siSSB DAR1) and AOCs conjugated with the lipid C14, C16, C18, or C20 as well as oleyl, and carboxylic acid-modified lipids at position 3 nucleotide or position 6 nucleotide (pos.3 or pos.6) with 2′-triazole (“tz”) of the passenger strand of the SSB siRNA (mTfR1-mAb-pos6.tzC14.siSSB, mTfR1-mAb-pos6.tzC16.siSSB, mTfR1-mAb-pos6.tzC18.siSSB, mTfR1-mAb-pos6.tzC20.siSSB, mTfR1-mAb-pos6.tzC18-oleyl.siSSB, mTfR1-mAb-pos6.tzC18-int.acid.siSSB, mTfR1-mAb-pos6.tzC20da.siSSB, mTfR1-mAb-pos.3.C16.pos6.C16.siSSB, mTfR1-mAb-pos6.C16.siSSB) at a dose of 0.5 mg/kg of siRNA.

FIGS. 10A-10C are bar graphs showing SSB siRNA tissue concentration in gastrocnemius (gastroc), tibialis anterior (TA) or heart tissue obtained from mice 28 days after a single intravenous injection of the control AOC (mTfR1-mAb-siSSB DAR1) and AOCs conjugated with the various lipids attached at position 3 nucleotide of the passenger strand (mTfR1-mAb-pos3.C16.siSSB, mTfR1-mAb-pos3.C22.siSSB, mTfR1-mAb-pos3.tzC14.siSSB, mTfR1-mAb-pos3.tzC16.siSSB, mTfR1-mAb-pos3.tzC18.siSSB, mTfR1-mAb-pos3.tzC20.siSSB, mTfR1-mAb-pos3.tzC18-oleyl.siSSB, mTfR1-mAb-pos3.tzC18-int.acid.siSSB) at a dose of 0.5 mg/kg of siRNA.

FIG. 10A is a bar graph showing SSB siRNA tissue concentration in gastrocnemius (gastroc) tissue obtained from mice 28 days after a single intravenous injection of the control AOC (mTfR1-mAb-siSSB DAR1) and AOCs conjugated with various lipids attached at position 3 nucleotide of the passenger strand (mTfR1-mAb-pos3.C16.siSSB, mTfR1-mAb-pos3.C22.siSSB, mTfR1-mAb-pos3.tzC14.siSSB, mTfR1-mAb-pos3.tzC16.siSSB, mTfR1-mAb-pos3.tzC18.siSSB, mTfR1-mAb-pos3.tzC20.siSSB, mTfR1-mAb-pos3.tzC18-oleyl.siSSB, mTfR1-mAb-pos3.tzC18-int.acid.siSSB) at a dose of 0.5 mg/kg of siRNA.

FIG. 10B are bar graphs showing SSB siRNA tissue concentration in tibialis anterior (TA) tissue obtained from mice 28 days after a single intravenous injection of the control AOC (mTfR1-mAb-siSSB DAR1) and AOCs conjugated with various lipids attached at position 3 nucleotide of the passenger strand (mTfR1-mAb-pos3.C16.siSSB, mTfR1-mAb-pos3.C22.siSSB, mTfR1-mAb-pos3.tzC14.siSSB, mTfR1-mAb-pos3.tzC16.siSSB, mTfR1-mAb-pos3.tzC18.siSSB, mTfR1-mAb-pos3.tzC20.siSSB, mTfR1-mAb-pos3.tzC18-oleyl.siSSB, mTfR1-mAb-pos3.tzC18-int.acid.siSSB) at a dose of 0.5 mg/kg of siRNA.

FIG. 10C are bar graphs showing SSB siRNA tissue concentration in heart tissue obtained from mice 28 days after a single intravenous injection of the control AOC (mTfR1-mAb-siSSB DAR1) and AOCs conjugated with various lipids attached at position 3 nucleotide of the passenger strand (mTfR1-mAb-pos3.C16.siSSB, mTfR1-mAb-pos3.C22.siSSB, mTfR1-mAb-pos3.tzC14.siSSB, mTfR1-mAb-pos3.tzC16.siSSB, mTfR1-mAb-pos3.tzC18.siSSB, mTfR1-mAb-pos3.tzC20.siSSB, mTfR1-mAb-pos3.tzC18-oleyl.siSSB, mTfR1-mAb-pos3.tzC18-int.acid.siSSB) at a dose of 0.5 mg/kg of siRNA.

FIGS. 11A-11C are bar graphs showing siRNA expression levels in gastrocnemius (gastroc), tibialis anterior (TA), or heart tissue obtained from CD1 mice 28 days after a single intravenous injection of the control AOC (mTfR1-mAb-siSSB DAR1) or AOCs conjugated with various lipids attached at position 3 nucleotide of the passenger strand (mTfR1-mAb-pos3.C16.siSSB, mTfR1-mAb-pos3.C22.siSSB, mTfR1-mAb-pos3.tzC14.siSSB, mTfR1-mAb-pos3.tzC16.siSSB, mTfR1-mAb-pos3.tzC18.siSSB, mTfR1-mAb-pos3.tzC20.siSSB, mTfR1-mAb-pos3.tzC18-oleyl.siSSB, mTfR1-mAb-pos3.tzC18-int.acid.siSSB) at a dose of 0.5 mg/kg of siRNA.

FIG. 11A are bar graphs showing siRNA expression levels in gastrocnemius (gastroc) tissue obtained from CD1 mice 28 days after a single intravenous injection of the control AOC (mTfR1-mAb-siSSB DAR1) and AOCs conjugated with various lipids attached at position 3 nucleotide of the passenger strand (mTfR1-mAb-pos3.C16.siSSB, mTfR1-mAb-pos3.C22.siSSB, mTfR1-mAb-pos3.tzC14.siSSB, mTfR1-mAb-pos3.tzC16.siSSB, mTfR1-mAb-pos3.tzC18.siSSB, mTfR1-mAb-pos3.tzC20.siSSB, mTfR1-mAb-pos3.tzC18-oleyl.siSSB, mTfR1-mAb-pos3.tzC18-int.acid.siSSB) at a dose of 0.5 mg/kg of siRNA.

FIG. 11B is a bar graph showing siRNA expression levels in tibialis anterior (TA) tissue obtained from CD1 mice 28 days after a single intravenous injection of the control AOC (mTfR1-mAb-siSSB DAR1) and AOCs conjugated with various lipids attached at position 3 nucleotide of the passenger strand (mTfR1-mAb-pos3.C16.siSSB, mTfR1-mAb-pos3.C22.siSSB, mTfR1-mAb-pos3.tzC14.siSSB, mTfR1-mAb-pos3.tzC16.siSSB, mTfR1-mAb-pos3.tzC18.siSSB, mTfR1-mAb-pos3.tzC20.siSSB, mTfR1-mAb-pos3.tzC18-oleyl.siSSB, mTfR1-mAb-pos3.tzC18-int.acid.siSSB) at a dose of 0.5 mg/kg of siRNA.

FIG. 11C is a bar graph showing siRNA expression levels in heart tissue obtained from CD1 mice 28 days after a single intravenous injection of the control AOC (mTfR1-mAb-siSSB DAR1) and AOCs conjugated with various different lipids attached at position 3 nucleotide of the passenger strand (mTfR1-mAb-pos3.C16.siSSB, mTfR1-mAb-pos3.C22.siSSB, mTfR1-mAb-pos3.tzC14.siSSB, mTfR1-mAb-pos3.tzC16.siSSB, mTfR1-mAb-pos3.tzC18.siSSB, mTfR1-mAb-pos3.tzC20.siSSB, mTfR1-mAb-pos3.tzC18-oleyl.siSSB, mTfR1-mAb-pos3.tzC18-int.acid.siSSB) at a dose of 0.5 mg/kg of siRNA.

FIGS. 12A-12F are bar graphs showing siRNA tissue concentrations in gastrocnemius (gastroc), tibialis anterior (TA), and heart tissue obtained from CD1 mice 28 days or 84 days after a single intravenous injection the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 12A is a bar graph showing siRNA tissue concentrations in gastrocnemius (gastroc) tissue obtained from CD1 mice 28 days after a single intravenous injection the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 12B is a bar graph showing siRNA tissue concentrations in tibialis anterior (TA) tissue obtained from CD1 mice 28 days after a single intravenous injection the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 12C is a bar graph showing siRNA tissue concentrations in heart tissue obtained from CD1 mice 28 days after a single intravenous injection the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 12D is a bar graph showing siRNA tissue concentrations in gastrocnemius (gastroc) tissue obtained from CD1 mice 84 days after a single intravenous injection the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 12E is a bar graph showing siRNA tissue concentrations in tibialis anterior (TA) tissue obtained from CD1 mice 84 days after a single intravenous injection the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 12F is a bar graph showing siRNA tissue concentrations in heart tissue obtained from CD1 mice 84 days after a single intravenous injection the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIGS. 13A-13F are graphs showing mRNA expression levels in gastrocnemius (gastroc), tibialis anterior (TA), and heart tissue obtained from CD1 mice 28 days or 84 days after a single intravenous injection of the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 13A are graphs showing mRNA expression levels in gastrocnemius (gastroc) tissue obtained from CD1 mice 28 days after a single intravenous injection of the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 13B are graphs showing mRNA expression levels in tibialis anterior (TA) tissue obtained from CD1 mice 28 days after a single intravenous injection of the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 13C are graphs showing mRNA expression levels in heart tissue obtained from CD1 mice 28 days after a single intravenous injection of the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 13D are graphs showing mRNA expression levels in gastrocnemius (gastroc), tibialis anterior (TA), and heart tissue obtained from CD1 mice 84 days after a single intravenous injection of the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 13E are graphs showing mRNA expression levels in tibialis anterior (TA) tissue obtained from CD1 mice 84 days after a single intravenous injection of the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIG. 13F are graphs showing mRNA expression levels in heart tissue obtained from CD1 mice 84 days after a single intravenous injection of the control DAR1 AOC (mTfR1-mAb-siSSB) and DAR1 lipid AOCs with C6, C10, C16, or C22 lipids at position 3 nucleotide (pos.3) of the passenger strand of the SSB siRNA (mTfR1-pos.3.C6.siSSB, mTfR1-pos.3.C10.siSSB, mTfR1-pos.3.C16.siSSB, or mTfR1-pos.3.C22.siSSB) or at position 6 nucleotide of the SSB siRNA (mTfR1-pos.6.C6.siSSB, mTfR1-pos.6.C10.siSSB, mTfR1-pos.6.C16.siSSB or mTfR1-pos.6.C22.siSSB) at a dose of 0.5 mg/kg of siRNA.

FIGS. 14A-14C are bar graphs showing siRNA tissue concentrations in gastrocnemius (gastroc), tibialis anterior (TA) and liver tissue obtained from CD1-mice 2 days after a single intravenous injection of the control DAR2 AOC (mTfR1-mAb-siMSTN) and DAR2 lipid AOCs conjugated with C16 lipid at position 6 nucleotide (mTfR1-mAb-pos6.C16.siMSTN) or position 19 nucleotide (mTfR1-mAb-pos19.C16.siMSTN) of the passenger strand of the MSTN siRNA.

FIG. 14A is a bar graph showing siRNA tissue concentrations in gastrocnemius (gastroc) tissue obtained from CD1 mice 2 days after a single intravenous injection of the control DAR2 AOC (mTfR1-mAb-siMSTN) and DAR2 lipid AOCs conjugated with C16 lipid at position 6 nucleotide (mTfR1-mAb-pos6.C16.siMSTN) or position 19 nucleotide (mTfR1-mAb-pos19.C16.siMSTN) of the passenger strand of the MSTN siRNA.

FIG. 14B is a bar graph showing siRNA tissue concentrations in tibialis anterior (TA) tissue obtained from CD1 mice 2 days after a single intravenous injection of the control DAR2 AOC (mTfR1-mAb-siMSTN) and DAR2 lipid AOCs conjugated with C16 lipid at position 6 v(mTfR1-mAb-pos6.C16.siMSTN) or position 19 nucleotide (mTfR1-mAb-pos19.C16.siMSTN) of the passenger strand of the MSTN siRNA.

FIG. 14C is a bar graph showing siRNA tissue concentrations in liver tissue obtained from CD1 mice 2 days after a single intravenous injection of the control DAR2 AOC (mTfR1-mAb-siMSTN) and DAR2 lipid AOCs conjugated with C16 lipid at position 6 nucleotide (mTfR1-mAb-pos6.C16.siMSTN) or position 19 nucleotide (mTfR1-mAb-pos19.C16.siMSTN) of the passenger strand of the MSTN siRNA.

FIGS. 15A-15B are bar graphs showing siRNA tissue concentrations in gastrocnemius (gastroc) and tibialis anterior (TA) obtained from CD1 mice 28 days after a single intravenous injection of the control DAR1/2 AOCs (mTfR1-mAb-siMSTN, mTfR1-mAb-2X4PG.siMSTN) and DAR1/2 lipid AOCs conjugated with C16 lipid at position 6 nucleotide (mTfR1-mAb-pos6.C16.siMSTN) or position 19 nucleotide (mTfR1-mAb-pos19.C16.siMSTN) of the passenger strand of the MSTN siRNA, or C16 lipid at position 6 nucleotide of the passenger strand of MSTN siRNA with PG modifications (mTfR1-mAb-2X4PG.pos6.C16.siMSTN)

FIG. 15A is a bar graph showing siRNA tissue concentrations in gastrocnemius (gastroc) tissue obtained from CD1 mice 28 days after a single intravenous injection of the control DAR1/2 AOCs (mTfR1-mAb-siMSTN, mTfR1-mAb-2X4PG.siMSTN) and DAR1/2 lipid AOCs conjugated with C16 lipid at position 6 nucleotide (mTfR1-mAb-pos6.C16.siMSTN) or position 19 nucleotide (mTfR1-mAb-pos19.C16.siMSTN) of the passenger strand of the MSTN siRNA, or C16 lipid at position 6 nucleotide of the passenger strand of MSTN siRNA with PG modifications (mTfR1-mAb-2X4PG.pos6.C16.siMSTN).

FIG. 15B is a bar graph showing siRNA tissue concentrations in tibialis anterior (TA) tissue obtained from CD1 mice 28 days after a single intravenous injection of the control DAR1/2 AOCs (mTfR1-mAb-siMSTN, mTfR1-mAb-2X4PG.siMSTN) and DAR1/2 lipid AOCs conjugated with C16 lipid at position 6 nucleotide (mTfR1-mAb-pos6.C16.siMSTN) or position 19 nucleotide (mTfR1-mAb-pos19.C16.siMSTN) of the passenger strand of the MSTN siRNA, or C16 lipid at position 6 nucleotide of the passenger strand of MSTN siRNA with PG modifications (mTfR1-mAb-2X4PG.pos6.C16.siMSTN).

FIGS. 16A-16B are bar graphs showing mRNA expression levels in gastrocnemius (gastroc), tibialis anterior (TA) obtained from CD1 mice 28 days after a single intravenous injection of the control DAR1/2 AOCs (mTfR1-mAb-siMSTN, mTfR1-mAb-2X4PG.siMSTN) and DAR1/2 lipid AOCs conjugated with C16 lipid at position 6 nucleotide (mTfR1-mAb-pos6.C16.siMSTN) or position 19 nucleotide (mTfR1-mAb-pos19.C16.siMSTN) of the passenger strand of the MSTN siRNA, or C16 lipid at position 6 nucleotide of the passenger strand of MSTN siRNA with PG modifications (mTfR1-mAb-2X4PG.pos6.C16.siMSTN).

FIG. 16A is a bar graph showing mRNA expression levels in gastrocnemius (gastroc) tissue obtained from CD1 mice 28 days after a single intravenous injection of the control DAR1/2 AOCs (mTfR1-mAb-siMSTN mTfR1-mAb-2X4PG.siMSTN) and DAR1/2 lipid AOCs conjugated with C16 lipid at position 6 nucleotide (mTfR1-mAb-pos6.C16.siMSTN) or position 19 nucleotide (mTfR1-mAb-pos19.C16.siMSTN) of the passenger strand of the MSTN siRNA, or C16 lipid at position 6 nucleotide of the passenger strand of MSTN siRNA with PG modifications (mTfR1-mAb-2X4PG.pos6.C16.siMSTN.

FIG. 16B is bar graph showing mRNA expression levels in tibialis anterior (TA) tissue obtained from CD1 mice 28 days after a single intravenous injection of the control DAR1/2 AOCs (mTfR1-mAb-siMSTN, mTfR1-mAb-2X4PG.siMSTN) and DAR1/2 lipid AOCs conjugated with C16 lipid at position 6 nucleotide (mTfR1-mAb-pos6.C16.siMSTN) or position 19 nucleotide (mTfR1-mAb-pos19.C16.siMSTN) of the passenger strand of the MSTN siRNA, or C16 lipid at position 6 nucleotide of the passenger strand of MSTN siRNA with PG modifications (mTfR1-mAb-2X4PG.pos6.C16.siMSTN.

FIGS. 17A-17D are bar graphs showing siRNA tissue concentrations in gastrocnemius (gastroc), tibialis anterior (TA), heart, and liver tissue obtained from CD1 mice 2 days after a single intravenous injection of the control DAR1/2 AOCs (mTfR1-mAb-siMSTN) and DAR2/4/5 lipid AOCs conjugated with C16 lipid at position 6 nucleotide (mTfR1-mAb-pos6.C16.siMSTN) or C22 lipid at position 3 nucleotide (mTfR1-mAb-pos3.C22.siMSTN) of the passenger strand of the MSTN siRNA.

FIG. 17A is a bar graph showing siRNA tissue concentrations in gastrocnemius (gastroc) tissue obtained from CD1 mice 2 days after a single intravenous injection of the control DAR1/2 AOCs (mTfR1-mAb-siMSTN) and DAR2/4/5 lipid AOCs conjugated with C16 lipid at position 6 (mTfR1-mAb-pos6.C16.siMSTN) or C22 lipid at position 3 nucleotide (mTfR1-mAb-pos3.C22.siMSTN) of the passenger strand of the MSTN siRNA.

FIG. 17B are bar graphs showing siRNA tissue concentrations in tibialis anterior (TA) tissue obtained from CD1 mice 2 days after a single intravenous injection of the control DAR1/2 AOCs (mTfR1-mAb-siMSTN) and DAR2/4/5 lipid AOCs conjugated with C16 lipid at position 6 (mTfR1-mAb-pos6.C16.siMSTN) or C22 lipid at position 3 nucleotide (mTfR1-mAb-pos3.C22.siMSTN) of the passenger strand of the MSTN siRNA.

FIG. 17C are bar graphs showing siRNA tissue concentrations in heart tissue obtained from CD1 mice 2 days after a single intravenous injection of the control DAR1/2 AOCs (mTfR1-mAb-siMSTN) and DAR2/4/5 lipid AOCs conjugated with C16 lipid at position 6 (mTfR1-mAb-pos6.C16.siMSTN) or C22 lipid at position 3 nucleotide (mTfR1-mAb-pos3.C22.siMSTN) of the passenger strand of the MSTN siRNA.

FIG. 17D are bar graphs showing siRNA tissue concentrations in liver tissue obtained from CD1 mice 2 days after a single intravenous injection of the control DAR1/2 AOCs (mTfR1-mAb-siMSTN) and DAR2/4/5 lipid AOCs conjugated with C16 lipid at position 6 (mTfR1-mAb-pos6.C16.siMSTN) or C22 lipid at position 3 nucleotide (mTfR1-mAb-pos3.C22.siMSTN) of the passenger strand of the MSTN siRNA.

FIGS. 18A-18E are plots illustrating binding affinity using ELISA of an anti-transferrin receptor monoclonal antibody (mTfR1-mAb), DAR1/2/4/5 AOC (mTfR1-mAb-siMSTN), DAR1/2/4/5 lipid AOCs conjugated with 2′-O—C16 lipid at position 6 nucleotide of the passenger strand position 6 (mTfR1-mAb-pos6.C16.siMSTN), and DAR1/2 lipid AOCs conjugated with 2′-O—C22 lipid modified at position 3 nucleotide of the passenger strand (mTfR1-mAb-pos3.C22.siMSTN).

FIG. 18A are plots illustrating binding affinity using ELISA of an anti-transferrin receptor monoclonal antibody (mTfR1-mAb), a DAR1 AOC (mTfR1-mAb-siMSTN), DAR1 lipid AOCs conjugated with 2′-O—C16 lipid modified at passenger strand's position 6 nucleotide (mTfR1-mAb-pos6.C16.siMSTN).

FIG. 18B are plots illustrating binding affinity using ELISA of an anti-transferrin receptor monoclonal antibody (mTfR1-mAb), a DAR2 AOC (mTfR1-mAb-siMSTN), DAR2 lipid AOCs conjugated with 2′-O—C16 lipid modified at passenger strand's position 6 nucleotide(mTfR1-mAb-pos6.C16.siMSTN).

FIG. 18C are plots illustrating binding affinity using ELISA of an anti-transferrin receptor monoclonal antibody (mTfR1-mAb), DAR2 lipid AOCs conjugated with 2′-O—C16 lipid modified at passenger strand's position 6 nucleotide (mTfR1-mAb-pos6.C16.siMSTN).

FIG. 18D are plots illustrating binding affinity using ELISA of an anti-transferrin receptor monoclonal antibody (mTfR1-mAb), DAR4 and DAR5 lipid AOCs conjugated with 2′-0-C16 lipid modified at passenger strand's position 6 nucleotide (mTfR1-mAb-pos6.C16.siMSTN).

FIG. 18E are plots illustrating binding affinity using ELISA of an anti-transferrin receptor monoclonal antibody (mTfR1-mAb), DAR1 and DAR2 lipid AOCs conjugated with 2′-0-C22 lipid modified at passenger strand's position 3 nucleotide (mTfR1-mAb-pos3.C22.siMSTN).

FIGS. 19A-19D are bar graphs showing siRNA tissue concentrations in gastrocnemius (gastroc), tibialis anterior (TA), heart, and liver tissue obtained from CD1 mice 28 days after a single intravenous injection of the control DAR1/2 AOCs (mTfR1-mAb-siMSTN) or the DAR2/4/5 lipid AOC (mTfR1-mAb-siMSTN) conjugated with C22 lipid at position 3 nucleotide (mTfR1-mAb-pos3.C22siMSTN) or C16 lipid at position 6 nucleotide (mTfR1-mAb-pos6.C16.siMSTN) of the passenger strand of the MSTN siRNA.

FIG. 19A is bar graphs showing siRNA tissue concentrations in gastrocnemius (gastroc) tissue obtained from CD1 mice 28 days after a single intravenous injection of the control DAR1/2 AOCs (mTfR1-mAb-siMSTN), DAR1/2/4/5 lipid AOC conjugated with C16 lipid at position 6 nucleotide (mTfR1-mAb-pos6.C16.siMSTN) of the passenger strand of the MSTN siRNA, and DAR1/2 lipid AOC conjugated with C22 lipid at position 3 nucleotide (mTfR1-mAb-pos3.C22siMSTN) of the passenger strand of the MSTN siRNA.

FIG. 19B is a bar graph showing siRNA tissue concentrations in tibialis anterior (TA) obtained from CD1 mice 28 days after a single intravenous injection of the control DAR1/2 AOCs (mTfR1-mAb-siMSTN), DAR1/2/4/5 lipid AOC conjugated with C16 lipid at position 6 nucleotide (mTfR1-mAb-pos6.C16.siMSTN) of the passenger strand of the MSTN siRNA, and DAR1/2 lipid AOC conjugated with C22 lipid at position 3 nucleotide (mTfR1-mAb-pos3.C22siMSTN) of the passenger strand of the MSTN siRNA.

FIG. 19C is a bar graph showing siRNA tissue concentrations in heart tissue obtained from CD1 mice 28 days after a single intravenous injection of the control DAR1/2 AOCs (mTfR1-mAb-siMSTN), DAR1/2/4/5 lipid AOC conjugated with C16 lipid at position 6 nucleotide (mTfR1-mAb-pos6.C16.siMSTN) of the passenger strand of the MSTN siRNA, and DAR1/2 lipid AOC conjugated with C22 lipid at position 3 nucleotide (mTfR1-mAb-pos3.C22siMSTN) of the passenger strand of the MSTN siRNA.

FIG. 19D is a bar graphs showing siRNA tissue concentrations in liver tissue obtained from CD1 mice 28 days after a single intravenous injection of the control DAR1/2 AOCs (mTfR1-mAb-siMSTN), DAR1/2/4/5 lipid AOC conjugated with C16 lipid at position 6 nucleotide (mTfR1-mAb-pos6.C16.siMSTN) of the passenger strand of the MSTN siRNA, and DAR1/2 lipid AOC conjugated with C22 lipid at position 3 nucleotide (mTfR1-mAb-pos3.C22siMSTN) of the passenger strand of the MSTN siRNA.

FIGS. 20A-20B are bar graphs showing mRNA expression levels in gastrocnemius (gastroc), tibialis anterior (TA) tissue obtained from CD1 mice 28 days after a single intravenous injection of the control DAR1/2 AOCs (mTfR1-mAb-siMSTN), DAR1/2/4/5 lipid AOC conjugated with C16 lipid at position 6 nucleotide (mTfR1-mAb-pos6.C16.siMSTN) of the passenger strand of the MSTN siRNA, and DAR1/2 lipid AOC conjugated with C22 lipid at position 3 nucleotide (mTfR1-mAb-pos3.C22siMSTN) of the passenger strand of the MSTN siRNA.

FIG. 20A is a bar graph showing mRNA expression levels in gastrocnemius (gastroc) tissue obtained from CD1 mice 28 days after a single intravenous injection of the control DAR1/2 AOCs (mTfR1-mAb-siMSTN), DAR1/2/4/5 lipid AOC conjugated with C16 lipid at position 6 nucleotide (mTfR1-mAb-pos6.C16.siMSTN) of the passenger strand of the MSTN siRNA, and DAR1/2 lipid AOC conjugated with C22 lipid at position 3 nucleotide (mTfR1-mAb-pos3.C22siMSTN) of the passenger strand of the MSTN siRNA.

FIG. 20B is a bar graph showing mRNA expression levels in tibialis anterior (TA) tissue obtained from CD1 mice 28 days after a single intravenous injection of the control DAR1/2 AOCs (mTfR1-mAb-siMSTN), DAR1/2/4/5 lipid AOC conjugated with C16 lipid at position 6 nucleotide (mTfR1-mAb-pos6.C16.siMSTN) of the passenger strand of the MSTN siRNA, and DAR1/2 lipid AOC conjugated with C22 lipid at position 3 nucleotide (mTfR1-mAb-pos3.C22siMSTN) of the passenger strand of the MSTN siRNA.

FIGS. 21A-21F are bar graphs showing siRNA tissue concentrations in gastrocnemius (gastroc), quadriceps, heart, liver, and kidney obtained from humanized TfR1 mice 28 days following a single intravenous (IV) injection of the control FabOCs and lipid FabOCs at a concentrations of 0.6 mg/kg siRNA or 1.2 mg/kg siRNA dose for DAR1 and DAR2, respectively.

FIG. 21A are bar graphs showing siRNA tissue concentration in gastrocnemius (gastroc), quadriceps, and heart obtained from humanized TfR1 mice 28 days following a single intravenous (IV) injection of the control FabOCs and lipid FabOCs at a concentration of 0.6 mg/kg siRNA or 1.2 mg/kg siRNA dose for DAR1 and DAR2, respectively. Addition of the C16 to passenger strand at position 6 increased the accumulation of siRNA concentrations compared to the non-lipid Fab control. Importantly, the addition of the C16 lipid doubled the concentration of siRNA in the target tissues going from DAR1 to DAR2. However, for the non-lipid Fab DAR2, no increase in muscle tissue siRNA concentration was observed.

FIG. 21B are bar graphs showing siRNA tissue concentration in liver and kidney obtained from humanized TfR1 mice 28 days following a single intravenous (IV) injection of the control FabOCs and lipid FabOCs at a concentration of 0.6 mg/kg siRNA or 1.2 mg/kg siRNA dose for DAR1 and DAR2, respectively. Addition of the C16 lipid to passenger strand at position 6 nucleotide decreased the siRNA concentrations in the kidney compared to the non-lipid Fab control.

FIG. 21C are bar graphs showing siRNA tissue concentration in gastrocnemius (gastroc), quadriceps, and heart obtained from humanized TfR1 mice 28 days following a single subcutaneous (SC) injection of the control FabOCs and lipid FabOCs at a concentration of 0.6 mg/kg siRNA or 1.2 mg/kg siRNA dose for DAR1 and DAR2, respectively. Addition of the C16 lipid to passenger strand at position 6 nucleotide increased the accumulation of siRNA concentrations compared to the non-lipid Fab controls.

FIG. 21D are bar graphs showing siRNA tissue concentrations in liver and kidney obtained from humanized TfR1 mice 28 days following a single subcutaneous (SC) injection of the control FabOCs and lipid FabOCs at a concentration of 0.6 mg/kg siRNA or 1.2 mg/kg siRNA dose for DAR1 and DAR2, respectively. Addition of the C16 to passenger strand at position 6 decreased the siRNA concentrations in the kidney compared to the non-lipid Fab control.

FIG. 21E are bar graphs showing DMPK mRNA expression levels in gastrocnemius (gastroc), quadriceps, and heart muscle tissues obtained from humanized TfR1 mice tissues 28 days following a single intravenous (IV) injection of the control FabOCs and lipid FabOCs at a concentration of 0.6 mg/kg siRNA or 1.2 mg/kg siRNA dose for DAR1 and DAR2, respectively.

FIG. 21F are bar graphs showing DMPK mRNA expression levels in gastrocnemius (gastroc), quadriceps, and heart muscle tissues obtained from humanized TfR1 mice 28 days following a single subcutaneous (SC) injection of the control FabOCs and lipid FabOCs at a dose of 0.6 mg/kg siRNA or 1.2 mg/kg siRNA dose for DAR1 and DAR2, respectively. Addition of the C16 lipid to passenger strand at position 6 nucleotide decreased DMPK mRNA expression compared to the non-lipid FabOC controls for both DAR1 and DAR2.

FIGS. 22A-22C are bar graphs illustrating siRNA tissue concentrations in gastrocnemius (gastroc), quadriceps, heart, liver, kidney tissues or DMPK mRNA expression levels in gastrocnemius (gastroc), quadriceps, or heart tissues obtained from humanized TfR1 mice 28 days following a single intravenous (IV) or subcutaneous (SC) injection of the control FabOCs and lipid FabOCs at a dose of 0.6 mg/kg siRNA for DAR1 or 1.2 mg/kg siRNA dose for DAR2.

FIG. 22A is a bar graph illustrating siRNA tissue concentrations in gastrocnemius (gastroc), quadriceps, and heart obtained from humanized TfR1 mice 28 days following a single intravenous (IV) or subcutaneous (SC) injection of the control FabOCs and lipid FabOCs at a dose of 0.6 mg/kg siRNA for DAR1 or 1.2 mg/kg siRNA dose for DAR2.

FIG. 22B a bar graph illustrating siRNA tissue concentrations in liver and kidney obtained from humanized TfR1 mice 28 days following a single intravenous (IV) or subcutaneous (SC) injection of the control FabOCs and lipid FabOCs at a dose of 0.6 mg/kg siRNA for DAR1 or 1.2 mg/kg siRNA dose for DAR2.

FIG. 22C is a bar graph illustrating DMPK mRNA expression levels in gastrocnemius (gastroc), quadriceps, and heart muscle tissues obtained from humanized TfR1 mice 28 days following a single intravenous (IV) injection of the control FabOCs and lipid FabOCs at a dose of 0.6 mg/kg siRNA for DAR1 or 1.2 mg/kg siRNA dose for DAR2.

FIGS. 23A-23B are bar graphs illustrating siRNA tissue concentration in tibialis anterior (TA), gastrocnemius (gastroc), and heart tissues or DMPK mRNA expression levels in tibialis anterior (TA), gastrocnemius (gastroc), and heart tissues obtained from humanized TfR1 mice 28 days following a single intravenous (IV) injection of DAR1 FabOC control (mTfR1-Fab-(vp)siDMPK), DAR1 lipid FabOCs with C16 lipid at position 6 of the passenger strand of the siRNA (mTfR1-Fab-(vp)pos6.C16.siDMPK and mTfR1-(C16)Fab-(vp)siDMPK) or with lipid covalently attached to the linker (mTfR1-Fab-C16.linker-(vp)siDMPK) linking the Fab to the siRNA.

FIG. 23A are bar graphs illustrating siRNA tissue concentration in tibialis anterior (TA), gastrocnemius (gastroc), and heart tissues obtained from wild type mice 28 days following a single intravenous (IV) injection of DAR1 FabOC control (mTfR1-Fab-(vp)siDMPK), DAR1 lipid FabOCs with C16 lipid at position 6 nucleotide of the passenger strand of the siRNA (mTfR1-Fab-(vp)pos6.C16.siDMPK and mTfR1-(C16)Fab-(vp)siDMPK) or with lipid covalently attached to the linker (mTfR1-Fab-C16.linker-(vp)siDMPK) linking the Fab to the siRNA.

FIG. 23B are bar graphs illustrating DMPK mRNA expression levels in tibialis anterior (TA), gastrocnemius (gastroc), and heart tissues obtained from wild type mice 28 days following a single intravenous (IV) injection of DAR1 lipid FabOC control (mTfR1-Fab-(vp)siDMPK) or DAR1 lipid FabOCs with C16 lipid at position 6 nucleotide of the passenger strand of the siRNA (mTfR1-Fab-(vp)pos6.C16.siDMPK and mTfR1-(C16)Fab-(vp)siDMPK) or with lipid covalently attached to the linker (mTfR1-Fab-C16.linker-(vp)siDMPK) linking the Fab to the siRNA.

FIG. 24A-24B shows representative synthesis schemes of the lipid linkers described herein.

FIGS. 25A-25B are bar graphs illustrating siRNA tissue concentrations in gastrocnemius (gastroc), and heart tissues obtained from wild type mice after intravenous administration of DAR1 lipid-FabOCs with C16, C20, or C22 hydrocarbon chain.

FIG. 26A-26B are bar graphs illustrating DMPK mRNA expression levels in gastrocnemius (gastroc), and heart tissues obtained from wild type mice after intravenous administration of DAR1 lipid-FabOCs with C16, C20, or C22 hydrocarbon chain.

FIGS. 27A-27B are plots illustrating siRNA tissue concentrations in gastrocnemius (gastroc), and heart tissues obtained from wild type mice after intravenous administration of DAR1, DAR2, or DAR4 lipid-FabOCs with C16 or C22 hydrocarbon chain.

FIG. 28A-28B are bar graphs illustrating DMPK mRNA expression levels in gastrocnemius (gastroc), and heart tissues obtained from wild type mice after intravenous administration of DAR1, DAR2, or DAR4 lipid-FabOCs with C16 or C22 hydrocarbon chain.

FIGS. 29A-29C are plots illustrating siRNA tissue concentrations in gastrocnemius (gastroc), tibialis anterior (TA), and heart tissues obtained from humanized TfR1 mice after intravenous or subcutaneous administration of DAR1 lipid-FabOCs with C22 hydrocarbon chain compared to a control.

FIGS. 29D-29J are bar graphs illustrating DMPK mRNA expression levels in gastrocnemius (gastroc), tibialis anterior (TA), and heart tissues obtained from humanized TfR1 mice after subcutaneous administration of DAR1 lipid-FabOCs with C22 hydrocarbon chain compared to a control.

FIGS. 30A-30C are plots illustrating DMPK mRNA expression levels in gastrocnemius (gastroc), tibialis anterior (TA), and heart tissues obtained from humanized TfR1 mice after subcutaneous administration of DAR1 lipid-FabOCs with C22 hydrocarbon chain compared to a control.

FIGS. 31A-31F are bar graphs illustrating siRNA tissue concentrations and DMPK mRNA expression levels in gastrocnemius (gastroc), tibialis anterior (TA), and heart tissues obtained from humanized TfR1 mice after intravenous administration of DAR1 and DAR2 lipid-FabOCs with C18, C20, or C22 hydrocarbon chain compared to a control.

FIGS. 32A-32B are bar graphs illustrating DMPK mRNA expression levels in gastrocnemius (gastroc), tibialis anterior (TA), and heart tissues obtained from humanized TfR1 mice after intravenous administration of DAR1 and DAR2 lipid-FabOCs with C18, C20, or C22 hydrocarbon chain compared to a control.

FIGS. 32C-32E are bar graphs illustrating DMPK mRNA expression levels in gastrocnemius (gastroc), kidney, and liver tissues obtained from humanized TfR1 mice after intravenous administration of DAR1 and DAR2 lipid-FabOCs with C18, C20, or C22 hydrocarbon chain compared to a control.

FIGS. 33A-33E are bar graphs illustrating SSB mRNA expression levels and DMPK mRNA expression levels in gastrocnemius (gastroc), kidney, and liver tissues obtained from humanized TfR1 mice after intravenous administration of DAR1 and DAR2 lipid-FabOCs with C16 hydrocarbon chain compared to a control.

FIGS. 33F-33I are bar graphs illustrating SSB siRNA tissue concentrations and DMPK tissue concentrations in gastrocnemius (gastroc), kidney, and liver tissues obtained from humanized TfR1 mice after intravenous administration of DAR1 and DAR2 lipid-FabOCs with C16 hydrocarbon chain compared to a control.

FIGS. 34A-34E are bar graphs illustrating MSTN siRNA tissue concentrations and MSTN mRNA expression levels in gastrocnemius (gastroc), heart, kidney, and liver tissues obtained from humanized TfR1 mice after intravenous administration of DAR1 and DAR2 lipid-FabOCs with C16, C22, or C16×2 hydrocarbon chain(s) compared to a control.

FIGS. 35A-35K are plots and bar graphs illustrating DMPK mRNA expression levels and siRNA tissue concentrations in gastrocnemius (gastroc), vastus lateralis, bicep, liver, kidney, and heart muscle tissues obtained from cynomolgus monkeys after intravenous or subcutaneous administration of DAR1 and DAR2 lipid-FabOCs with C22 hydrocarbon chain compared to a control.

FIGS. 36A-36D are plots and bar graphs illustrating DMPK mRNA expression levels and siRNA tissue concentrations in gastrocnemius (gastric) and tibialis anterior (TA) obtained from wild type mice after intravenous administration of DAR1 lipid-FabOCs with cleavable or non-cleavable lipids.

FIGS. 37A-37B is a bar graph illustrating in vitro activity of DMPK siRNAs modified with C16 lipid at different positions of the passenger strand (FIG. 37A) and the guide strand (FIG. 37B), respectively.

FIG. 38 is a bar graph illustrating in vitro activity of DMPK siRNAs modified with degradable/cleavable or non-degradable/stable lipids.

FIG. 39 shows an exemplary structure of the lipid modified polynucleotide molecule described herein.

### DETAILED DESCRIPTION OF THE DISCLOSURE

Nucleic acid (e.g., RNAi) therapy is a targeted therapy with high selectivity and specificity. However, in some instances, nucleic acid therapy is also hindered by poor intracellular uptake, limited blood stability and non-specific immune stimulation. To address these issues, various modifications of the nucleic acid composition are explored, such as for example, novel linkers for better stabilizing and/or lower toxicity, optimization of binding moiety for increased target specificity and/or target delivery, and nucleic acid polymer modifications for increased stability and/or reduced off-target effect.

In some embodiments, the arrangement or order of the different components that make-up the nucleic acid composition further effects intracellular uptake, stability, toxicity, efficacy, and/or non-specific immune stimulation. For example, if the nucleic acid component includes a binding moiety, a polymer, and a polynucleic acid molecule (or polynucleotide), the order or arrangement of the binding moiety, the polymer, and/or the polynucleic acid molecule (or polynucleotide) (e.g., binding moiety-polynucleic acid molecule-polymer, binding moiety-polymer-polynucleic acid molecule, or polymer-binding moiety-polynucleic acid molecule) further effects intracellular uptake, stability, toxicity, efficacy, and/or non-specific immune stimulation.

In some embodiments, disclosed herein is a polynucleotide conjugate comprising a binding moiety conjugated to a polynucleotide molecule. In some embodiments, the polynucleotide conjugate further comprises a lipophilic moiety. In some embodiments, the lipophilic moiety is a linear or branched alkyl group. In some embodiments, the linear alkyl group is selected from the group consisting of C6, C8, C10, C12, C14, C16, C18, C20, C22, C24 and C26 hydrocarbon chain. In some embodiments, the linear alkyl group is a C16 or C22 hydrocarbon chain.

In some embodiments, described herein include a nucleic acid conjugate molecule which arrangement of components effects intracellular uptake, stability, toxicity, efficacy, and/or non-specific immune stimulation. In some instances, the nucleic acid conjugate molecule comprises a binding moiety conjugated to a polynucleic acid molecule and a polymer. In some embodiments, the molecule comprises a molecule according to Formula (I):

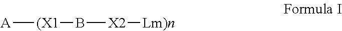

wherein,
   A is a binding moiety;
   B is a polynucleotide;
   L is a lipophilic moiety;
   X1 is a bond or a linker;
   X2 is a bond or a linker;
   m≥1;
   n≥1;
where the lipid moiety is conjugated to the polynucleotide and the binding moiety is conjugated to the polynucleotide.

In some embodiments, the lipophilic moiety is an aliphatic, cyclic such as alicyclic, or polycyclic such as polyalicyclic compound, such as a steroid (e.g., sterol) or a linear or branched aliphatic hydrocarbon. In some instances, the lipophilic moiety comprises a hydrocarbon chain, which may be cyclic or acyclic. In some instances, the hydrocarbon chain comprises various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₂₂ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, in some instances, the lipophilic moiety comprises a C₄-C₃₀ hydrocarbon chain (e.g., C₄-C₃₀ alkyl or alkenyl). In some embodiments, the lipophilic moiety comprises a saturated or unsaturated C₆-C₁₈ hydrocarbon chain (e.g., a linear C₆-C₂₂ alkyl or alkenyl). In one embodiment, the lipophilic moiety comprises a saturated or unsaturated C16 hydrocarbon chain (e.g., a linear C16 alkyl or alkenyl). In one embodiment, the lipophilic moiety comprises a saturated or unsaturated C₂₂ hydrocarbon chain (e.g., a linear C₂₂ alkyl or alkenyl). In one embodiment, the lipophilic moiety is attached to the 2′-OH of a nucleotide. In one embodiment, the lipophilic moiety is a lipid having an unsaturated C16 hydrocarbon chain (C16). In one embodiment, the lipophilic moiety is a lipid having an unsaturated C22 hydrocarbon chain (C22).

In some embodiments, the lipophilic moiety is conjugated to the polynucleotide. In some embodiments, the polynucleotide comprises a single stranded nucleic acid molecule. In some embodiments, the polynucleotide comprises two or more strands of nucleic acid molecules. In some embodiments, the polynucleotide comprises a first polynucleotide and a second polynucleotide hybridized to the first polynucleotide to form a double-stranded polynucleic acid molecule. In some embodiments, the first polynucleotide and the second polynucleotide are RNA molecules. In some instances, the polynucleotide is a double-stranded nucleic acid molecule (e.g., siRNA). In some embodiments, the polynucleotide comprises two distinct nucleic acid sequences targeting the same gene and having synergistic effects on the targeted gene.

In some instances, the polynucleic acid molecule is a double-stranded polynucleotide molecule comprising sense and antisense strands, where the antisense region comprises nucleic acid sequence that is partially or fully complementary to nucleic acid sequence in a target nucleic acid molecule or a portion thereof and the sense region having nucleotide sequence partially or fully corresponding to the target nucleic acid sequence or a portion thereof. In some instances, the polynucleic acid molecule is assembled from two separate polynucleotides, where one strand is the sense strand and the other is the antisense strand, wherein the antisense and sense strands are self-complementary (e.g., each strand comprises nucleotide sequence that is complementary to nucleotide sequence in the other strand; such as where the antisense strand and sense strand form a duplex or double stranded structure, for example wherein the double stranded region is about 19, 20, 21, 22, 23, or more base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the polynucleic acid molecule is assembled from a single oligonucleotide, where the self-complementary sense and antisense regions of the polynucleic acid molecule are linked by means of a nucleic acid based or non-nucleic acid-based linker(s).

In some cases, the polynucleic acid molecule is a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. In other cases, the polynucleic acid molecule is a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide is processed either in vivo or in vitro to generate an active polynucleic acid molecule capable of mediating RNAi. In additional cases, the polynucleic acid molecule also comprises a single-stranded polynucleotide having nucleotide sequence complementary to nucleotide sequence in a target nucleic acid molecule or a portion thereof (for example, where such polynucleic acid molecule does not require the presence within the polynucleic acid molecule of nucleotide sequence corresponding to the target nucleic acid sequence or a portion thereof), wherein the single stranded polynucleotide further comprises a terminal phosphate group, such as a 5′-phosphate (see for example Martinez et al., 2002, Cell, 110, 563-574 and Schwarz et al., 2002, Molecular Cell, 10, 537-568), or 5′,3′-diphosphate.

In some instances, an asymmetric hairpin is a linear polynucleic acid molecule comprising an antisense region, a loop portion that comprises nucleotides or non-nucleotides, and a sense region that comprises fewer nucleotides than the antisense region to the extent that the sense region has enough complimentary nucleotides to base pair with the antisense region and form a duplex with loop. For example, an asymmetric hairpin polynucleic acid molecule comprises an antisense region having length sufficient to mediate RNAi in a cell or in vitro system (e.g. about 19 to about 22 nucleotides) and a loop region comprising about 4 to about 8 nucleotides, and a sense region having about 3 to about 18 nucleotides that are complementary to the antisense region. In some cases, the asymmetric hairpin polynucleic acid molecule also comprises a 5′-terminal phosphate group that is chemically modified. In additional cases, the loop portion of the asymmetric hairpin polynucleic acid molecule comprises nucleotides, non-nucleotides, linker molecules, or conjugate molecules.

In some aspects, an asymmetric duplex is a polynucleic acid molecule having two separate strands comprising a sense region and an antisense region, wherein the sense region comprises fewer nucleotides than the antisense region to the extent that the sense region has enough complimentary nucleotides to base pair with the antisense region and form a duplex. For example, an asymmetric duplex polynucleic acid molecule comprises an antisense region having length sufficient to mediate RNAi in a cell or in vitro system (e.g., about 19 to about 22 nucleotides) and a sense region having about 3 to about 18 nucleotides that are complementary to the antisense region.

In some cases, a universal base refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

In some embodiments, the polynucleotide comprises at least one 2′ modified nucleotide. In some embodiments, the at least one 2′ modified nucleotide comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In some embodiments, the at least one 2′ modified nucleotide comprises locked nucleic acid (LNA) or ethylene nucleic acid (ENA). In some embodiments, the at least one modified internucleotide linkage comprises a phosphorothioate linkage, a phosphorodithioate linkage or a phosphoryl guanidine (PG) linkage. In certain embodiments, the polynucleic acid molecule comprises a 5′-terminal vinylphosphonate modified nucleotide, such as those described in U.S. Publication No. 2019/0192681. In some embodiments, the at least one inverted abasic moiety is at least one terminus.

In some instances, the lipophilic moiety is conjugated to the polynucleotide via linker. In some instance, the linker contains an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, X1 and X2 are independently a bond or a linker group. In some embodiments, X1 is a bond. In some embodiments, X1 is a C₁-C₆ alkyl group. In some embodiments, X2 is a C₁-C₆ alkyl group. In some embodiments, X1 is a homobifunctional linker or a heterobifunctional linker, optionally conjugated to a C₁-C₆ alkyl group. In some embodiments, X2 is a homobifunctional linker or a heterobifunctional linker. In some instances, the X2 is selected from a group of linkers listed in Table 1.

In some embodiments, the binding moiety is an antibody or antigen binding fragment thereof. In some embodiments, the antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), a single-arm antibody, diabody, minibody, nanobody, single-domain antibody (sdAb), or camelid antibody or antigen binding fragment thereof. In some embodiments, the antibody or antigen binding fragment thereof is an anti-TfR antibody or antigen binding fragment thereof. In some embodiments, the antibody or antigen binding fragment thereof is a Fab fragment. In some embodiments, the antibody or antigen binding fragment thereof is a VHH. In some embodiments, the antibody or antigen binding fragment thereof is a Fab-Fc fusion antibody. In some embodiments, the antibody or antigen binding fragment thereof is a VHH-Fc fusion antibody.

In some embodiments, the binding moiety is a multicyclic peptide. In some embodiments, the binding moiety is a bicyclic or tricyclic peptide. In some embodiments, the binding moiety is a bicyclic or tricyclic peptide specifically binds to a transferrin receptor (TfR), preferably, specifically binds to transferrin receptor 1 (TfR1), or more preferably, specifically binds to human transferrin receptor 1 (TfR1) (or human CD71).

Also disclosed herein, in certain embodiments, is a molecule of Formula (II):

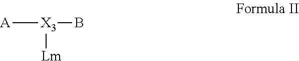

wherein,
   A is a binding moiety;
   B is a polynucleotide;
   L is a lipophilic moiety;
   X₃ is a linker;
   m≥1;
where the lipophilic moiety is conjugated to the linker and the binding moiety is conjugated to the polynucleotide.

In some embodiments, the lipophilic moiety is an aliphatic, cyclic such as alicyclic, or polycyclic such as polyalicyclic compound, such as a steroid (e.g., sterol) or a linear or branched aliphatic hydrocarbon. In some instances, the lipophilic moiety comprises a hydrocarbon chain, which may be cyclic or acyclic. In some instances, the hydrocarbon chain comprises various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₂₂ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, in some instances, the lipophilic moiety comprises a C₄-C₃₀ hydrocarbon chain (e.g., C₄-C₃₀ alkyl or alkenyl). In some embodiments, the lipophilic moiety comprises a saturated or unsaturated C₆-C₁₈ hydrocarbon chain (e.g., a linear C₆-C₂₂ alkyl or alkenyl). In one embodiment, the lipophilic moiety comprises a saturated or unsaturated C16 hydrocarbon chain (e.g., a linear C16 alkyl or alkenyl). In one embodiment, the lipophilic moiety comprises a saturated or unsaturated C₂₂ hydrocarbon chain (e.g., a linear C₂₂ alkyl or alkenyl). In one embodiment, the lipophilic moiety is attached to the 2′-OH of a nucleotide. In one embodiment, the lipophilic moiety is a lipid having an unsaturated C16 hydrocarbon chain (C16). In one embodiment, the lipophilic moiety is a lipid having an unsaturated C₂₂ hydrocarbon chain (C₂₂).

In some embodiments, the polynucleotide comprises at least one 2′ modified nucleotide that includes 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In some embodiments, the at least one 2′ modified nucleotide comprises locked nucleic acid (LNA) or ethylene nucleic acid (ENA). In some embodiments, the polynucleotide comprises at least one modified internucleotide linkage that includes a phosphorothioate linkage, a phosphorodithioate linkage or a phosphoryl guanidine (PG) linkage. In certain embodiments, the polynucleic acid molecule comprises a 5′-terminal vinylphosphonate modified nucleotide, such as those described in U.S. Publication No. 2019/0192681. In some embodiments, the polynucleotide comprises at least one inverted abasic moiety is at least one terminus.

In some embodiments, the polynucleotide comprises a single stranded molecule. In some embodiments, the polynucleotide comprises two or more strands. In some embodiments, the polynucleotide comprises a first polynucleotide and a second polynucleotide hybridized to the first polynucleotide to form a double-stranded polynucleic acid molecule. In some embodiments, the first polynucleotide and the second polynucleotide are RNA molecules. In some instances, the polynucleotide is a double-stranded nucleic acid molecule (e.g., siRNA). In some embodiments, the polynucleotide comprises two distinct nucleic acid sequences targeting the same gene and having synergistic effects on the targeted gene. In some embodiments, X3 is a bond or a linker group. In some embodiments, X3 is a bond. In some embodiments, X3 is a C₁-C₆ alkyl group. In some embodiments, X3 is a homobifunctional linker or a heterobifunctional linker, optionally conjugated to a C₁-C₆ alkyl group. In some instances, the X3 is selected from a group of linkers listed in Table 1.

In some embodiments, the binding moiety is an antibody or antigen binding fragment thereof. In some embodiments, the antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), a single-arm antibody, diabody, minibody, nanobody, single-domain antibody (sdAb), or camelid antibody or antigen binding fragment thereof. In some embodiments, the antibody or antigen binding fragment thereof is an anti-TfR antibody or antigen binding fragment thereof. In some embodiments, the antibody or antigen binding fragment thereof is a Fab fragment. In some embodiments, the antibody or antigen binding fragment thereof is a VHH. In some embodiments, the antibody or antigen binding fragment thereof is a Fab-Fc fusion antibody. In some embodiments, the antibody or antigen binding fragment thereof is a VHH-Fc fusion antibody.

In some embodiments, the binding moiety is a multicyclic peptide. In some embodiments, the binding moiety is a bicyclic or tricyclic peptide. In some embodiments, the binding moiety is a bicyclic or tricyclic peptide specifically binds to a transferrin receptor (TfR), preferably, specifically binds to transferrin receptor 1 (TfR1), or more preferably, specifically binds to human transferrin receptor 1 (TfR1) (or human CD71).

### Polynucleic Acid Molecules

In some embodiments, the polynucleic acid molecule hybridizes to a target region on a gene. In some instances, the polynucleic acid molecule comprises a nucleic acid sequence that is at least 70%, 75%, 80%, 85%, 90%, 95%, 98%, 99%, or 100% complementary to an equal length portion of the target region. In some instances, the hybridization is a high stringent hybridization condition. In some embodiments, the polynucleic acid molecule hybridizes to at least 8, 9, 10, 11, 12, 13, 14, 15, 16, 17, 18, 19, 20, or more contiguous bases of a target sequence described herein. In some embodiments, the sequence of the polynucleic acid molecule has 5 or less mismatches to a target sequence described herein. In some embodiments, the sequence of the polynucleic acid molecule has 4 or less mismatches to a target sequence described herein. In some instances, the sequence of the polynucleic acid molecule may have 3 or less mismatches to a target sequence described herein. In some cases, the sequence of the polynucleic acid molecule may have 2 or less mismatches to a target sequence described herein. In some cases, the sequence of the polynucleic acid molecule may have 1 or less mismatches to a target sequence described herein.

In some embodiments, the polynucleic acid molecule described herein comprises RNA or DNA. In some cases, the polynucleic acid molecule comprises RNA. In some instances, RNA comprises short interfering RNA (siRNA), short hairpin RNA (shRNA), microRNA (miRNA), double-stranded RNA (dsRNA), transfer RNA (tRNA), ribosomal RNA (rRNA), small activating RNA (saRNA), or heterogeneous nuclear RNA (hnRNA). In some instances, RNA comprises shRNA. In some instances, RNA comprises miRNA. In some instances, RNA comprises dsRNA. In some instances, RNA comprises tRNA. In some instances, RNA comprises rRNA. In some instances, RNA comprises hnRNA. In some embodiments, the polynucleic acid molecule is a double-stranded polynucleic acid molecule. In some instances, the polynucleic acid molecule comprises siRNA. In some cases, the polynucleic acid molecule comprises siRNA. In some embodiments, the polynucleic acid molecule described herein comprises dsRNA binding enzymes, RNA-specific adenosine deaminase (ADAR).

In some embodiments, the polynucleic acid molecule is a single-stranded polynucleic acid molecule. In some embodiments, the polynucleic acid molecule is an ASO or a PMO. In some embodiments, the polynucleic acid molecule described herein comprises PMO and ASO for splice switching.

In some embodiments, the polynucleic acid molecule is a double-stranded polynucleic acid molecule. In some embodiments, the double-stranded polynucleic acid molecule comprises a passenger strand comprising a PMO molecule and a guide strand comprising an RNA molecule, forming a heteroduplex structure. In some instances, the double-stranded polynucleic acid molecule comprises a blunt terminus, an unmatched terminus, an overhang, or a combination thereof. In some instances, the blunt terminus is a 5′ blunt terminus, a 3′ blunt terminus, or both. In some instances, the unmatched terminus is a 5′ unmatched terminus, a 3′ unmatched terminus, or both. In some instances, the overhang is a 5′ overhang, a 3′ overhang, or both. In some cases, the overhang comprises about 1-4 non-base pairing nucleotides. In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides.

In some embodiments, the polynucleic acid molecule is from about 10 to about 50 nucleotides in length. In some instances, the polynucleic acid molecule is from about 10 to about 30, from about 15 to about 30, from about 18 to about 25, from about 18 to about 24, from about 19 to about 23, or from about 20 to about 22 nucleotides in length.

In some embodiments, the polynucleic acid molecule is about 50 nucleotides in length. In some instances, the polynucleic acid molecule is about 45 nucleotides in length. In some instances, the polynucleic acid molecule is about 40 nucleotides in length. In some instances, the polynucleic acid molecule is about 35 nucleotides in length. In some instances, the polynucleic acid molecule is about 30 nucleotides in length. In some instances, the polynucleic acid molecule is about 25 nucleotides in length. In some instances, the polynucleic acid molecule is about 20 nucleotides in length. In some instances, the polynucleic acid molecule is about 19 nucleotides in length. In some instances, the polynucleic acid molecule is about 18 nucleotides in length. In some instances, the polynucleic acid molecule is about 17 nucleotides in length. In some instances, the polynucleic acid molecule is about 16 nucleotides in length. In some instances, the polynucleic acid molecule is about 15 nucleotides in length. In some instances, the polynucleic acid molecule is about 14 nucleotides in length. In some instances, the polynucleic acid molecule is about 13 nucleotides in length. In some instances, the polynucleic acid molecule is about 12 nucleotides in length. In some instances, the polynucleic acid molecule is about 11 nucleotides in length. In some instances, the polynucleic acid molecule is about 10 nucleotides in length. In some instances, the polynucleic acid molecule is from about 10 to about 50 nucleotides in length. In some instances, the polynucleic acid molecule is from about 10 to about 45 nucleotides in length. In some instances, the polynucleic acid molecule is from about 10 to about 40 nucleotides in length. In some instances, the polynucleic acid molecule is from about 10 to about 35 nucleotides in length. In some instances, the polynucleic acid molecule is from about 10 to about 30 nucleotides in length. In some instances, the polynucleic acid molecule is from about 10 to about 25 nucleotides in length. In some instances, the polynucleic acid molecule is from about 10 to about 20 nucleotides in length. In some instances, the polynucleic acid molecule is from about 15 to about 25 nucleotides in length. In some instances, the polynucleic acid molecule is from about 15 to about 30 nucleotides in length. In some instances, the polynucleic acid molecule is from about 12 to about 30 nucleotides in length.

In some embodiments, the polynucleic acid molecule comprises a first polynucleotide. In some instances, the polynucleic acid molecule comprises a second polynucleotide. In some instances, the polynucleic acid molecule comprises a first polynucleotide and a second polynucleotide. In some instances, the first polynucleotide is a sense strand or passenger strand. In some instances, the second polynucleotide is an antisense strand or guide strand.

In some embodiments, the polynucleic acid molecule comprises a first polynucleotide and/or a second polynucleotide. In some embodiments, the first polynucleotide or the second polynucleotide is from about 10 to about 50 nucleotides in length. In some instances, the first polynucleotide or the second polynucleotide is from about 10 to about 30, from about 15 to about 30, from about 18 to about 25, from about 18 to about 24, from about 19 to about 23, or from about 20 to about 22 nucleotides in length.

In some instances, the first polynucleotide or the second polynucleotide is about 50 nucleotides in length. In some instances, the first polynucleotide or the second polynucleotide is about 45 nucleotides in length. In some instances, the first polynucleotide or the second polynucleotide is about 40 nucleotides in length. In some instances, the first polynucleotide or the second polynucleotide is about 35 nucleotides in length. In some instances, the first polynucleotide or the second polynucleotide is about 30 nucleotides in length. In some instances, the first polynucleotide or the second polynucleotide is about 25 nucleotides in length. In some instances, the first polynucleotide or the second polynucleotide is about 20 nucleotides in length. In some instances, the first polynucleotide or the second polynucleotide is about 19 nucleotides in length. In some instances, the first polynucleotide or the second polynucleotide is about 18 nucleotides in length. In some instances, the first polynucleotide or the second polynucleotide is about 17 nucleotides in length. In some instances, the first polynucleotide or the second polynucleotide is about 16 nucleotides in length. In some instances, the first polynucleotide or the second polynucleotide is about 15 nucleotides in length. In some instances, the first polynucleotide or the second polynucleotide is about 14 nucleotides in length. In some instances, the first polynucleotide or the second polynucleotide is about 13 nucleotides in length. In some instances, the first polynucleotide or the second polynucleotide is about 12 nucleotides in length. In some instances, the first polynucleotide or the second polynucleotide is about 11 nucleotides in length. In some instances, the first polynucleotide or the second polynucleotide is about 10 nucleotides in length. In some instances, the first polynucleotide or the second polynucleotide is from about 10 to about 50 nucleotides in length. In some instances, the first polynucleotide or the second polynucleotide is from about 10 to about 45 nucleotides in length. In some instances, the first polynucleotide or the second polynucleotide is from about 10 to about 40 nucleotides in length. In some instances, the first polynucleotide or the second polynucleotide is from about 10 to about 35 nucleotides in length. In some instances, the first polynucleotide or the second polynucleotide is from about 10 to about 30 nucleotides in length. In some instances, the first polynucleotide or the second polynucleotide is from about 10 to about 25 nucleotides in length. In some instances, the first polynucleotide or the second polynucleotide is from about 10 to about 20 nucleotides in length. In some instances, the first polynucleotide or the second polynucleotide is from about 15 to about 25 nucleotides in length. In some instances, the first polynucleotide or the second polynucleotide is from about 15 to about 30 nucleotides in length. In some instances, the first polynucleotide or the second polynucleotide is from about 12 to about 30 nucleotides in length.

In some embodiments, the polynucleic acid molecule comprises a first polynucleotide and a second polynucleotide. In some instances, the polynucleic acid molecule further comprises a blunt terminus, an overhang, or a combination thereof. In some instances, the blunt terminus is a 5′ blunt terminus, a 3′ blunt terminus, or both. In some cases, the overhang is a 5′ overhang, 3′ overhang, or both. In some cases, the overhang comprises 1, 2, 3, 4, 5, 6, 7, 8, 9, or 10 non-base pairing nucleotides. In some cases, the overhang comprises 1, 2, 3, 4, 5, or 6 non-base pairing nucleotides. In some cases, the overhang comprises 1, 2, 3, or 4 non-base pairing nucleotides. In some cases, the overhang comprises 1 non-base pairing nucleotide. In some cases, the overhang comprises 2 non-base pairing nucleotides. In some cases, the overhang comprises 3 non-base pairing nucleotides. In some cases, the overhang comprises 4 non-base pairing nucleotides.

In some cases, the overhang is stable/non-degradable. In some cases, the overhang is degradable/cleavable by enzyme-mediated degradation mechanisms including nucleases. In some embodiments, the degradable overhang comprises a 5-Octadecynyl-2′-OMe-modified nucleotide. In some embodiments, the degradable overhang comprises a 5-Octadecynyl-2′-OMe-modified uridine. In some embodiments, the degradable overhang comprises a 5-Octadecynyl-2′-OMe-Uridine Phosphoramidite. In some embodiments, the degradable overhang comprises C18 5-U Phosphoramidite. An example of the degradable overhang described herein is shown below:

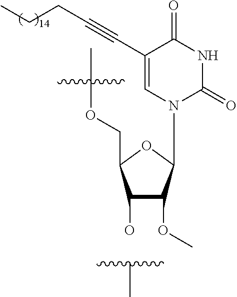

In some embodiments, the polynucleic acid molecule has reduced off-target effect. In some instances, “off-target” or “off-target effects” refer to any instance in which a polynucleic acid polymer directed against a given target causes an unintended effect by interacting either directly or indirectly with another mRNA sequence, a DNA sequence or a cellular protein or other moiety. In some instances, an “off-target effect” occurs when there is a simultaneous degradation of other transcripts due to partial homology or complementarity between that other transcript and the sense and/or antisense strand of the polynucleic acid molecule.

In some embodiments, the polynucleic acid molecule comprises natural, synthetic, or artificial nucleotide analogues or bases. In some cases, the polynucleic acid molecule comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some embodiments, a nucleotide analogue or artificial nucleotide base described above comprises a nucleic acid with a modification at a 2′ hydroxyl group or at a 3′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl (2′-O-MOE) modification. In some cases, the 2′-O-methyl modification adds a methyl group to the 2′ hydroxyl group of the ribose moiety whereas the 2′O-methoxyethyl modification adds a methoxyethyl group to the 2′ hydroxyl group of the ribose moiety. Exemplary chemical structures of a 2′-O-methyl modification of an adenosine molecule and 2′O-methoxyethyl modification of a uridine are illustrated below.

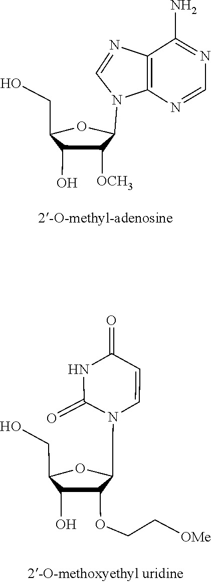

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties. An exemplary chemical structure of a 2′-O-aminopropyl nucleoside phosphoramidite is illustrated below.

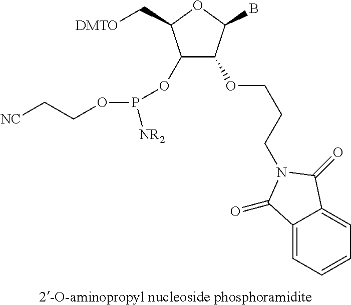

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer. Exemplary representations of the chemical structure of LNA are illustrated below. The representation shown to the left highlights the chemical connectivities of an LNA monomer. The representation shown to the right highlights the locked 3′-endo (³E) conformation of the furanose ring of an LNA monomer.

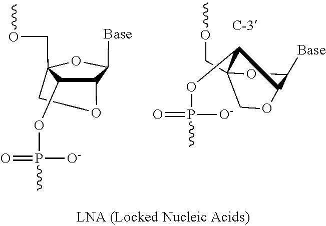

In some instances, the modification at the 2′ hydroxyl group comprises ethylene nucleic acids (ENA) such as for example 2′-4′-ethylene-bridged nucleic acid, which locks the sugar conformation into a C₃′-endo sugar puckering conformation. ENA are part of the bridged nucleic acids class of modified nucleic acids that also comprises LNA. Exemplary chemical structures of the ENA and bridged nucleic acids are illustrated below.

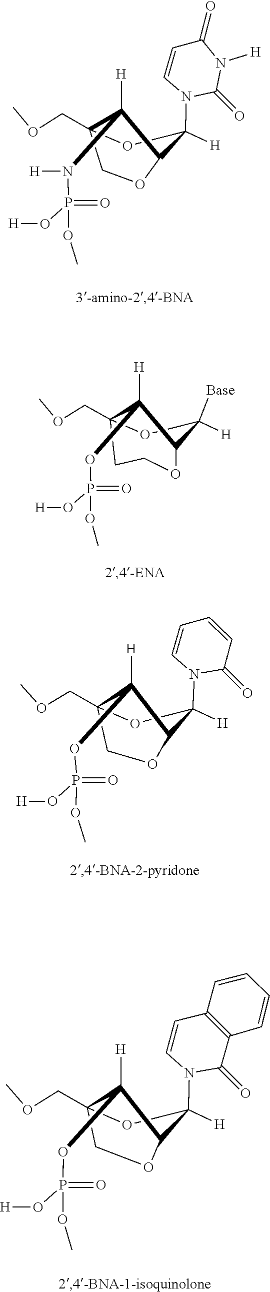

In some embodiments, additional modifications at the 2′ hydroxyl group include 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA).

In some embodiments, a nucleotide analogue comprises a modified base such as, but not limited to, 5-propynyluridine, 5-propynylcytidine, 6-methyladenine, 6-methylguanine, N, N, -dimethyladenine, 2-propyladenine, 2 propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O- and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes what are known in the art as universal bases. By way of example, universal bases include but are not limited to 3-nitropyrrole, 5-nitroindole, or nebularine. In some aspects, the heterocyclic base moiety is a modified base as described elsewhere herein.

In some embodiments, a nucleotide analogue further comprises a morpholino, a peptide nucleic acid (PNA), a methylphosphonate nucleotide, a thiolphosphonate nucleotide, a 2′-fluoro N3-P5′-phosphoramidite, or a 1′,5′-anhydrohexitol nucleic acid (HNA). Morpholino or phosphorodiamidate morpholino oligo (PMO) comprises synthetic molecules whose structure mimics natural nucleic acid structure but deviates from the normal sugar and phosphate structures. In some instances, the five member ribose ring is substituted with a six member morpholino ring containing four carbons, one nitrogen, and one oxygen. In some cases, the ribose monomers are linked by a phosphorodiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

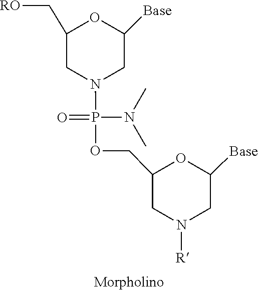

In some embodiments, a morpholino or PMO described above is a PMO comprising a positive or cationic charge. In some instances, the PMO is PMOplus (Sarepta). PMOplus refers to phosphorodiamidate morpholino oligomers comprising any number of (1-piperazino)phosphinylideneoxy, (1-(4-(omega-guanidino-alkanoyl))-piperazino)phosphinylideneoxy linkages (e.g., as such those described in PCT Publication No. WO2008/036127. In some cases, the PMO is a PMO described in U.S. Pat. No. 7,943,762.

In some embodiments, a morpholino or PMO described above is a PMO-X (Sarepta). In some cases, PMO-X refers to phosphorodiamidate morpholino oligomers comprising at least one linkage or at least one of the disclosed terminal modifications, such as those disclosed in PCT Publication No. WO2011/150408 and U.S. Publication No. 2012/0065169.

In some embodiments, a morpholino or PMO described above is a PMO as described in Table 5 of U.S. Publication No. 2014/0296321.

In some embodiments, peptide nucleic acid (PNA) does not contain sugar ring or phosphate linkage and the bases are attached and appropriately spaced by oligoglycine-like molecules, therefore, eliminating a backbone charge.

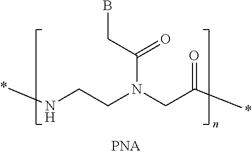

In some embodiments, one or more modifications occur at the internucleotide linkage. In some instances, modified internucleotide linkage includes, but is not limited to, phosphorothioates; phosphorodithioates; methylphosphonates; 5′-alkylenephosphonates; 5′-methylphosphonate; 3′-alkylene phosphonates; borontrifluoridates; borano phosphate esters and selenophosphates of 3-5′linkage or 2′-5′linkage; phosphotriesters; thionoalkylphosphotriesters; hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides; carbonates; carbamates; methylenehydrazos; methylenedimethylhydrazos; formacetals; thioformacetals; oximes; methyleneiminos; methylenemethyliminos; thioamidates; linkages with riboacetyl groups; aminoethyl glycine; silyl or siloxane linkages; alkyl or cycloalkyl linkages with or without heteroatoms of, for example, 1 to 10 carbons that are saturated or unsaturated and/or substituted and/or contain heteroatoms; linkages with morpholino structures, amides, or polyamides wherein the bases are attached to the aza nitrogens of the backbone directly or indirectly; and combinations thereof.

In some instances, an oligonucleotide disclosed herein comprises at least one internucleotide linkage represented by Formula (III):

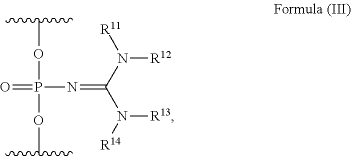

wherein,
each R¹¹, R¹², R¹³, and R¹⁴ is independently selected from —H, —C₁₋₁₀ alkyl, —C₂₋₁₀ alkenyl, —C₂₋₁₀ alkynyl, or —C₆₋₁₀ aryl;
optionally, wherein R¹² and R¹³, together with an atom to which they are bound, form a 5-8 membered heterocyclic substituent moiety, selected from the group consisting of N-pyrrolidinyl, N-piperidinyl, N-azepanyl, N-azocanyl, and imidazolidine.

In some aspects, R¹² and R¹³, together with an atom to which they are bound, form a 5-8 membered heterocyclic substituent moiety. In some aspects, R¹² and R¹³, together with an atom to which they are bound, form a 5-membered heterocyclic substituent moiety. In some aspects, R¹² and R¹³, together with an atom to which they are bound, form a 6-membered heterocyclic substituent moiety. In some aspects, R¹² and R¹³, together with an atom to which they are bound, form a 7-membered heterocyclic substituent moiety. In some aspects, R¹² and R¹³, together with an atom to which they are bound, form an 8-membered heterocyclic substituent moiety.

In some aspects, R¹² and R¹³, together with the atom to which they are bound to form N-pyrrolidinyl. In some aspects, R¹² and R¹³, together with the atom to which they are bound, form N-piperidinyl. In some aspects, R¹² and R¹³, together with the atom to which they are bound, form N-azepanyl. In some aspects, R¹² and R¹³, together with the atom to which they are bound, form N-azocanyl. In some aspects, R¹² and R¹³, together with the atom to which they are bound, form an imidazolidine.

In some aspects, each R¹¹, R¹², R¹³, and R¹⁴ is independently selected from —H, —C₁₋₁₀ alkyl, —C₂₋₁₀ alkenyl, —C₂₋₁₀ alkynyl, or —C₆₋₁₀ aryl. In some aspects, R¹¹ is selected from —H, —C₁₋₁₀ alkyl, —C₂₋₁₀ alkenyl, —C₂₋₁₀ alkynyl, or —C₆₋₁₀ aryl. In some aspects, R¹¹ is methyl. In some aspects, R¹² is selected from —H, —C₁₋₁₀ alkyl, —C₂₋₁₀ alkenyl, —C₂₋₁₀ alkynyl, or —C₆₋₁₀ aryl. In some aspects, R¹² is methyl. In some aspects, R¹³ is selected from —H, —C₁₋₁₀ alkyl, —C₂₋₁₀ alkenyl, —C₂₋₁₀ alkynyl, or —C₆₋₁₀ aryl. In some aspects, R¹³ is methyl. In some aspects, R¹⁴ is selected from —H, —C₁₋₁₀ alkyl, —C₂₋₁₀ alkenyl, —C₂₋₁₀ alkynyl, or —C₆₋₁₀ aryl. In some aspects, R¹⁴ is methyl.

In some aspects, R¹¹, R¹², R¹³, and R¹⁴ are —C₁₋₁₀ alkyl. In some aspects, R¹¹ is —C₁₋₁₀ alkyl. In some aspects, R¹¹ is methyl. In some aspects, R¹² is —C₁₋₁₀ alkyl. In some aspects, R¹² is methyl. In some aspects, R¹³ is —C₁₋₁₀ alkyl. In some aspects, R¹³ is methyl. In some aspects, R¹⁴ is —C₁₋₁₀ alkyl. In some aspects, R¹⁴ is methyl.

In some aspects, an oligonucleotide comprises at least one internucleotide linkage represented by Formula (IV):

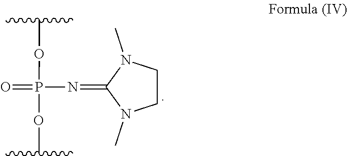 [SMILES: *OP(=O)(N=C1N(C)CCN1C)O*]

In some aspects, an oligonucleotide comprises at least one internucleotide linkage represented by Formula (V):

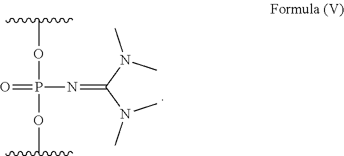 [SMILES: *OP(=O)(N=C(N(C)C)N(C)C)O*]

In some aspects, an oligonucleotide comprises at least one internucleotide linkage represented by Formula (VI):

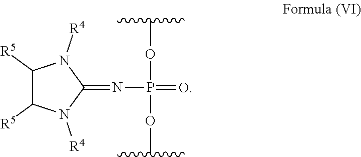

wherein,
each R⁴ is independently selected from hydrogen or C₁₋₁₀ alkyl; and
each R⁵ is independently selected from hydrogen, halogen, hydroxy, alkoxy, or C₁₋₁₀ alkyl.

In some aspects, an oligonucleotide comprises at least one internucleotide linkage represented by Formula (VI):

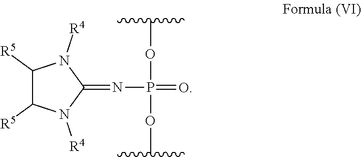

wherein each R⁴ is independently selected from hydrogen or C₁₋₁₀ alkyl; and
wherein the oligonucleotide comprises at least 12 nucleotides.

In some aspects, an oligonucleotide comprises a contiguous sequence of monomer subunits linked by internucleotide linking groups wherein at least one of the internucleotide linking groups is represented by Formula (VI):

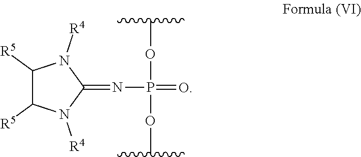

wherein,
each R⁴ is independently selected from hydrogen or C₁₋₁₀ alkyl; and
each R⁵ is independently selected from hydrogen, halogen, hydroxy, alkoxy, or C₁₋₁₀ alkyl.

In some aspects, a second oligonucleotide comprises a contiguous sequence of monomer subunits linked by internucleotide linking groups wherein at least one of the internucleotide linking groups is represented by Formula (VI):

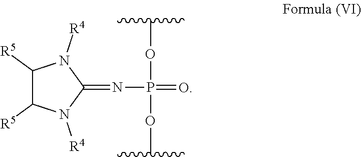

wherein,
each R⁴ is independently selected from hydrogen or C₁₋₁₀ alkyl; and
each R⁵ is independently selected from hydrogen, halogen, hydroxy, alkoxy, or C₁₋₁₀ alkyl.

In some aspects, an oligonucleotide includes at least one monomer subunit of the monomer subunits is represented by Formula (VI*):

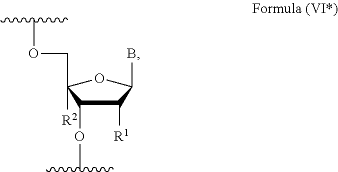

wherein,
   B is independently selected from a heterocyclic base moiety;
   R¹ is independently selected from hydrogen, hydroxy, halogen, or alkoxy;
   R² is independently hydrogen; and
   R¹ and R² optionally come together with the atoms to which they are bound to form a C₃-C₄ carbocycle.

In some aspects, an oligonucleotide includes at least two monomer subunits of the contiguous sequence are linked by Formula (VI) as represented by Formula (VII):

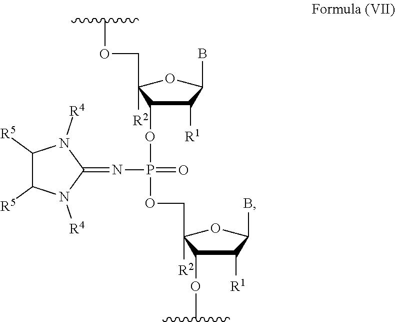

wherein,
each B is independently selected from a heterocyclic base moiety;
each R¹ is independently selected from hydrogen, hydroxy, halogen, or alkoxy;
each R² is independently hydrogen;
R¹ and R² optionally come together with the atoms to which they are bound to form a C₃-C₄ carbocycle;
each R⁴ is independently selected from hydrogen or C₁₋₁₀ alkyl; and
each R⁵ is independently selected from hydrogen, halogen, hydroxy, alkoxy, or C₁₋₁₀ alkyl.

In some aspects, each R¹ is independently selected from hydrogen, hydroxy, halogen, or alkoxy. In some cases, R¹ is hydrogen. In some cases, R¹ is hydroxy. In some cases, R¹ is halogen. In some cases, R¹ is fluorine. In some cases, R¹ is C₁₋₆ alkoxy.

In some aspects, each R⁴ is independently selected from hydrogen or C₁₋₆ alkyl. In some aspects, each R⁴ is independently selected from hydrogen. In some aspects, each R⁴ is independently selected from C₁₋₆ alkyl.

In some aspects, R¹ and R² optionally come together with the atoms to which they are bound to form a C₃ carbocycle. In some cases, R¹ and R² optionally come together with the atoms to which they are bound to form a C₄ carbocycle.

In some aspects, a modified internucleotide linkage is a phosphoryl guanidine (PG) linkage of Formula (III), (IV), (V) or (VI). In some aspects, the modified internucleotide linkage is a phosphoryl guanidine (PG) of Formula (III). In some aspects, the modified internucleotide linkage is a phosphoryl guanidine (PG) of Formula (IV). In some aspects, the modified internucleotide linkage is a phosphoryl guanidine (PG) of Formula (V). In some aspects, the modified internucleotide linkage is a phosphoryl guanidine (PG) of Formula (VI).

In some embodiments, the oligonucleotide comprises at least about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, or more modified internucleotide linkages of Formula (III), (IV), (V) or (VI).

In some cases, the oligonucleotide comprises at least about 1, about 2, about 3, about 4, about 5, about 6, about 7, about 8, about 9, about 10, about 11, about 12, about 13, about 14, about 15, or more modified internucleotide linkages of Formula (III), (IV), (V) or (VI).

In some cases, the oligonucleotide comprises at most about 1, about 2, about 3, about 4, about 5, about 6, about 7, about 8, about 9, about 10, about 11, about 12, about 13, about 14, about 15, or more modified internucleotide linkages of Formula (III), (IV), (V) or (VI). In some instances, the modified internucleotide linkages of Formula (III), (IV), (V) or (VI) are in tandem within the oligonucleotide. In other instances, the modified internucleotide linkages of Formula (III), (IV), (V) or (VI) are interspersed within the oligonucleotide, with nucleotides modified by one or more additional modifications. In some aspects, each internucleotide linkage is represented by Formula (III), (IV), (V) or (VI).

In some embodiments, the guide strand comprises 1, 2, 3, 4, 5, 6, 7, 8, 9, 10 PG internucleotide linkage and the passenger strand comprises 1, 2, 3, 4, 5, 6, 7, 8, 9, 10 PG internucleotide linkage. In some embodiments, the guide strand comprises 2 PG internucleotide linkage and the passenger strand comprises 4 PG internucleotide linkage.

In some instances, the modification is a methyl or thiol modification such as methylphosphonate or thiolphosphonate modification. Exemplary thiolphosphonate nucleotide (left) and methylphosphonate nucleotide (right) are illustrated below.

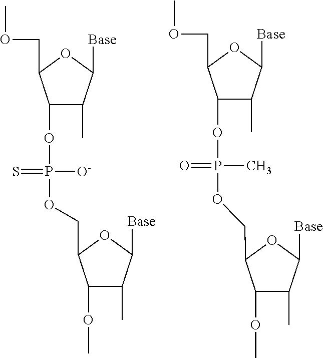

In some instances, a modified nucleotide includes, but is not limited to, 2′-fluoro N3-P5′-phosphoramidites illustrated as:

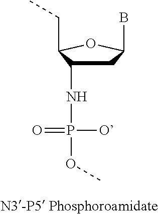

In some instances, a modified nucleotide includes, but is not limited to a 5′-vinylphosphonate modified non-natural nucleotide selected from:

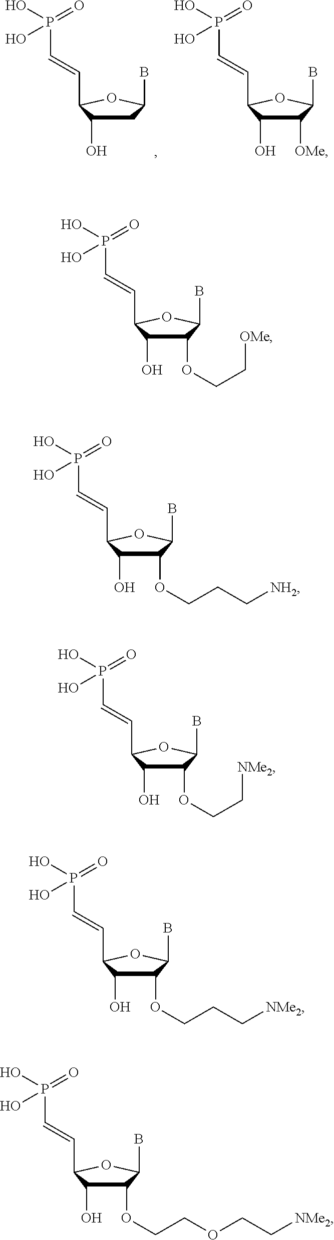

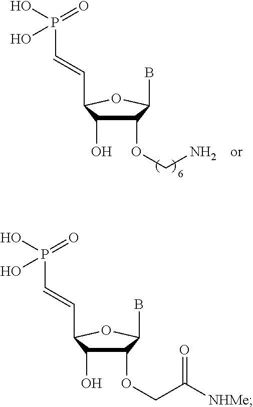

wherein B is a heterocyclic base moiety.

In some instances, a modified nucleotide includes, but is not limited to one 5′-vinylphosphonate modified non-natural nucleotide selected from:

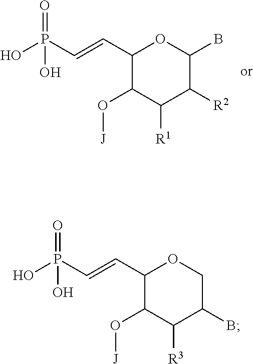

wherein B is a heterocyclic base moiety;
R1, R2, and R3 are independently selected from hydrogen, halogen, alkyl or alkoxy; and
J is an internucleotide linking group linking to an adjacent nucleotide of the oligonucleotide.

In some instances, a modified nucleotide includes, but is not limited to one 5′-vinylphosphonate modified non-natural nucleotide selected from:

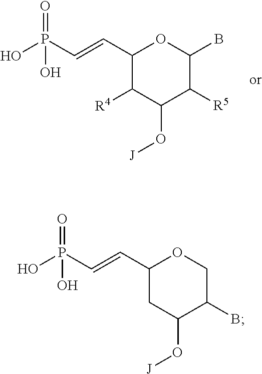

wherein B is a heterocyclic base moiety;
R4, and R5 are independently selected from hydrogen, halogen, alkyl or alkoxy; and
J is an internucleotide linking group linking to an adjacent nucleotide of the oligonucleotide.

In some instances, a modified nucleotide includes, but is not limited to one 5′-vinylphosphonate modified non-natural nucleotide selected from:

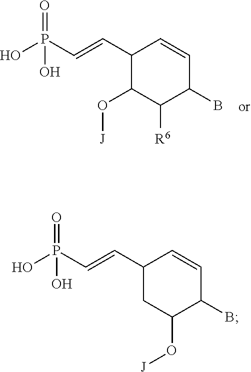

wherein B is a heterocyclic base moiety;
R6 is selected from hydrogen, halogen, alkyl or alkoxy; and
J is an internucleotide linking group linking to an adjacent nucleotide of the oligonucleotide.

In some instances, a modified nucleotide includes, but is not limited to one 5′-vinylphosphonate modified non-natural nucleotide selected from locked nucleic acid (LNA) or ethylene nucleic acid (ENA).

In some instances, a modified nucleotide includes, but is not limited to one 5′-vinylphosphonate modified non-natural nucleotide selected from:

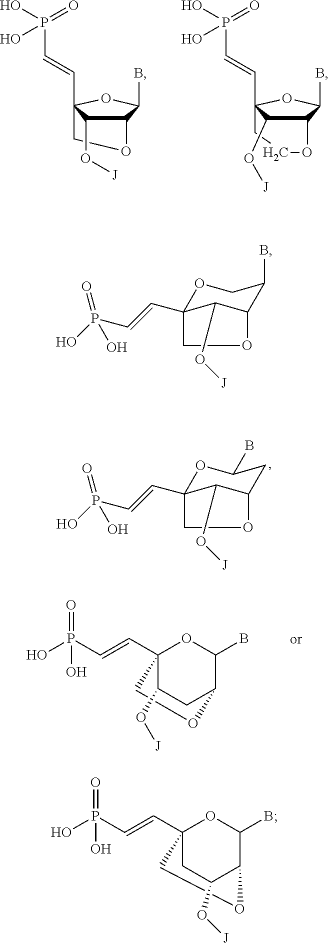

wherein B is a heterocyclic base moiety; and
J is an internucleotide linking group linking to an adjacent nucleotide of the oligonucleotide.

In some instances, a modified nucleotide includes, but is not limited to one 5′-vinylphosphonate modified non-natural nucleotide selected from:

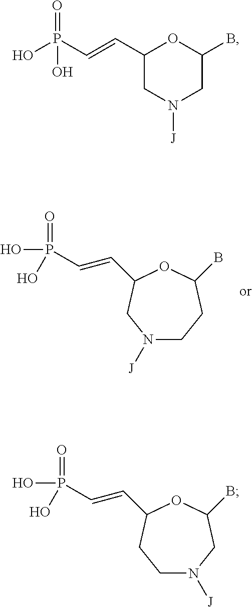

wherein B is a heterocyclic base moiety; and
J is an internucleotide linking group linking to an adjacent nucleotide of the oligonucleotide.

In some instances, a modified nucleotide includes, but is not limited to one 5′-vinylphosphonate modified non-natural nucleotide selected from:

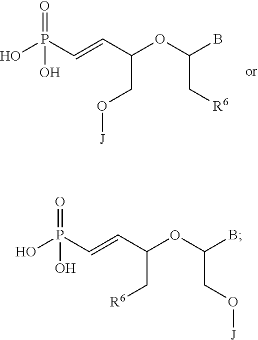

wherein B is a heterocyclic base moiety;
R6 is selected from hydrogen, halogen, alkyl or alkoxy; and
J is an internucleotide linking group linking to an adjacent nucleotide of the oligonucleotide.

In some instances, a modified nucleotide includes, but is not limited to one 5′-vinylphosphonate modified non-natural nucleotide is:

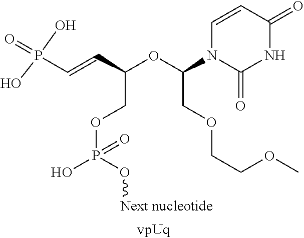

In some instances, a modified nucleotide includes, but is not limited to, hexitol nucleic acid (or 1′,5′-anhydrohexitol nucleic acids (HNA)) illustrated as:

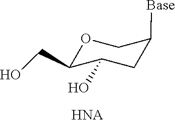

In some aspects, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some aspects, the polynucleic acid molecule comprises one or more of the artificial nucleotide analogues described herein. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues described herein. In some aspects, the artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-0-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of the artificial nucleotide analogues selected from 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof. In some instances, the polynucleic acid molecule comprises 1, 2, 3, 4, 5, 6, 7, 8, 9, 10, 11, 12, 13, 14, 15, 16, 17, 18, 20, 25, or more of 2′-O-methyl modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of 2′-O— methoxyethyl (2′-O-MOE) modified nucleotides. In some instances, the polynucleic acid molecule comprises 1, 2, 3, 4, 5, 6, 7, 8, 9, 10, 11, 12, 13, 14, 15, 16, 17, 18, 20, 25, or more of thiolphosphonate nucleotides.

In some instances, the polynucleic acid molecule comprises at least one of: from about 5% to about 100% modification, from about 10% to about 100% modification, from about 20% to about 100% modification, from about 30% to about 100% modification, from about 40% to about 100% modification, from about 50% to about 100% modification, from about 60% to about 100% modification, from about 70% to about 100% modification, from about 80% to about 100% modification, and from about 90% to about 100% modification.

In some aspects, the polynucleic acid molecule comprises at least 1, 2, 3, 4, 5, 6, 7, 8, 9, 10, 11, 12, 13, 14, 15, 16, 17, 18, 19, 20, 21, 22, 23, 24, 25 or more modifications or modified nucleotides, in which the modification comprises an artificial nucleotide analogue described herein.

In some instances, from about 5 to about 100% of the polynucleic acid molecule comprise the artificial nucleotide analogues described herein. In some instances, about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the polynucleic acid molecule comprise the artificial nucleotide analogues described herein. In some aspects, the artificial nucleotide analogues include 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-0-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or a combination thereof.

In some aspects, a polynucleic acid molecule comprises a sense strand and antisense strand, wherein pyrimidine nucleotides in the sense strand comprises 2′-O-methylpyrimidine nucleotides and purine nucleotides in the sense strand comprise 2′-deoxy purine nucleotides. In some aspects, a polynucleic acid molecule comprises a sense strand and antisense strand, wherein pyrimidine nucleotides present in the sense strand comprise 2′-deoxy-2′-fluoro pyrimidine nucleotides and wherein purine nucleotides present in the sense strand comprise 2′-deoxy purine nucleotides.

In some aspects, a polynucleic acid molecule comprises a sense strand and antisense strand, wherein the pyrimidine nucleotides when present in said antisense strand are 2′-deoxy-2′-fluoro pyrimidine nucleotides and the purine nucleotides when present in said antisense strand are 2′-O-methyl purine nucleotides.

In some aspects, a polynucleic acid molecule comprises a sense strand and antisense strand, wherein the pyrimidine nucleotides when present in said antisense strand are 2′-deoxy-2′-fluoro pyrimidine nucleotides and wherein the purine nucleotides when present in said antisense strand comprise 2′-deoxy-purine nucleotides.

In some aspects, a polynucleic acid molecule comprises a sense strand and antisense strand, and at least one of sense strand and antisense strands has a plurality of (e.g., two or more, three or more, four or more, five or more, six or more, seven or more, eight or more, etc.) 2′-O-methyl or 2′-deoxy-2′-fluoro modified nucleotides. In some aspects, where at least two out of the plurality of 2′-O-methyl or 2′-deoxy-2′-fluoro modified nucleotides are consecutive nucleotides. In some aspects, where consecutive 2′-O-methyl or 2′-deoxy-2′-fluoro modified nucleotides are located at the 5′-end of the sense strand and/or the antisense strand. In some aspects, where consecutive 2′-O-methyl or 2′-deoxy-2′-fluoro modified nucleotides are located at the 3′-end of the sense strand and/or the antisense strand. In some aspects, the sense strand of polynucleic acid molecule includes at least four, at least five, at least six consecutive 2′-O-methyl modified nucleotides at its 5′ end and/or 3′end, or both. Optionally, in such embodiments, the sense strand of polynucleic acid molecule includes at least one, at least two, at least three, at least four 2′-deoxy-2′-fluoro modified nucleotides at the 3′ end of the at least four, at least five, at least six consecutive 2′-O-methyl modified nucleotides at the polynucleotides' 5′ end, or at the 5′ end of the at least four, at least five, at least six consecutive 2′-O-methyl modified nucleotides at polynucleotides' 3′ end. Also optionally, such at least two, at least three, at least four 2′-deoxy-2′-fluoro modified nucleotides are consecutive nucleotides.

In some aspects, a polynucleic acid molecule comprises a sense strand and antisense strand, and at least one of sense strand and antisense strands has 2′-O-methyl modified nucleotide located at the 5′-end of the sense strand and/or the antisense strand. In some aspects, at least one of sense strand and antisense strands has 2′-O-methyl modified nucleotide located at the 3′-end of the sense strand and/or the antisense strand. In some aspects, the 2′-O-methyl modified nucleotide located at the 5′-end of the sense strand and/or the antisense strand is a purine nucleotide. In some aspects, the 2′-O-methyl modified nucleotide located at the 5′-end of the sense strand and/or the antisense strand is a pyridine nucleotide.

In some aspects, a polynucleic acid molecule comprises a sense strand and antisense strand, and the antisense strand has two or more consecutive 2′-deoxy-2′-fluoro modified nucleotides at 5′-end. In some aspects, a polynucleic acid molecule comprises a sense strand and antisense strand, and the antisense strand has two or more consecutive 2′-O-methyl modified nucleotides at 3′-end. In some aspects, a polynucleic acid molecule comprises a sense strand and antisense strand, and the antisense strand has at least 2, 3, 4, 5, 6, or 7 consecutive 2′-O-methyl modified nucleotides.

In some aspects, a polynucleic acid molecule comprises a sense strand and antisense strand, and the sense strand comprises a nucleic acid of 5′-nsnsnnnnNfNfNfnnnnnnnnsnsa-3′ (lower case (n)=2′-O-Me (methyl), Nf=2′-F (fluoro); s=phosphorothioate backbone modification). In some aspects, a polynucleic acid molecule comprises a sense strand and an antisense strand, and the antisense strand comprises a nucleic acid of 5′-nsNfsnnnNfnnnnnnnNfnNfnnnsusu-3′,5′-usNfsnnnNfnnnnnnnNfnNfnnnsusu-3′ or 5′-UfsNfsnnnNfnnnnnnnNfnNfnnnsusu-3′ (lower case (n)=2′-O-Me (methyl), Nf=2′-F (fluoro); s=phosphorothioate backbone modification). In some aspects, a polynucleic acid molecule comprises a sense strand and an antisense strand, and the sense strand comprises a nucleic acid of 5′-nsnsnnnnNfNfNfnnnnnnnnsnsa-3′ (lower case (n)=2′-O-Me (methyl), Nf=2′-F (fluoro); s=phosphorothioate backbone modification) and the antisense strand comprises a nucleic acid of 5′-nsNfsnnnNfnnnnnnnNfnNfnnnsnsn-3′,5′-usNfsnnnNfnnnnnnnNfnNfnnnsnsn-3′ or UfsNfsnnnNfnnnnnnnNfnNfnnnsnsn-3′ (lower case (n)=2′-O-Me (methyl), Nf=2′-F (fluoro); s=phosphorothioate backbone modification).

In some aspects, a polynucleic acid molecule comprises a sense strand and antisense strand, wherein the sense strand includes a terminal cap moiety at the 5′-end, the 3′-end, or both of the 5′ and 3′ ends of the sense strand. In other embodiments, the terminal cap moiety is an inverted deoxy abasic moiety.

In some aspects, a polynucleic acid molecule comprises a sense strand and an antisense strand, wherein the antisense strand comprises a glyceryl modification at the 3′ end of the antisense strand.

In some aspects, a polynucleic acid molecule comprises a sense strand and an antisense strand, in which the sense strand comprises one or more, for example, about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or about one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and in which the antisense strand comprises about 1 to about 10 or more, specifically about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without one or more, for example about 1, 2, 3, 4, 5, 6, 7, 8, 9, 10, or more, phosphorothioate internucleotide linkages and/or a terminal cap molecule at the 3′-end, the 5′-end, or both of the 3′- and 5′-ends, being present in the same or different strand.

In some aspects, a polynucleic acid molecule comprises a sense strand and an antisense strand, in which the sense strand comprises about 1 to about 25, for example, about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10, or more) 2′-deoxy, 2′-O-methyl, 2′-deoxy-2′-fluoro, and/or one or more (e.g., about 1, 2, 3, 4, 5, 6, 7, 8, 9, 10, or more) universal base modified nucleotides, and optionally a terminal cap molecule at the 3-end, the 5′-end, or both of the 3′- and 5′-ends of the sense strand; and in which the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, or more, pyrimidine nucleotides of the sense and/or antisense strand are chemically-modified with 2′-deoxy, 2′-O-methyl and/or 2′-deoxy-2′-fluoro nucleotides, with or without about 1 to about 25 or more, for example about 1, 2, 3, 4, 5, 6, 7, 8, 9, 10, 11, 12, 13, 14, 15, 16, 17, 18, 19, 20, or more phosphorothioate internucleotide linkages and/or a terminal cap molecule at the 3′-end, the 5′-end, or both of the 3′- and 5′-ends, being present in the same or different strand.

In some aspects, a polynucleic acid molecule comprises a sense strand and an antisense strand, in which the antisense strand comprises one or more, for example, about 1, 2, 3, 4, 5, 6, 7, 8, 9, 10, 11, 12, 13, 14, 15, 16, 17, 18, 19, 20, or more phosphorothioate internucleotide linkages, and/or about one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides at the 3′-end, the 5′-end, or both of the 3′- and 5′-ends of the sense strand and/or antisense strand, and optionally a terminal cap molecule at the 3′-end, the 5′-end, or both of the 3′- and 5′-ends of the sense strand. In some aspects, the antisense strand comprises about 1 to about 10 or more, specifically about 1, 2, 3, 4, 5, 6, 7, 8, 9, 10 or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10, 11, 12, 13, 14, 15, 16, 17, 18, 19, 20, or more pyrimidine nucleotides of the sense and/or antisense strand are chemically-modified with 2′-deoxy, 2′-O-methyl and/or 2′-deoxy-2′-fluoro nucleotides, with or without one or more, for example, about 1, 2, 3, 4, 5, 6, 7, 8, 9, 10 or more phosphorothioate internucleotide linkages and/or a terminal cap molecule at the 3′-end, the 5′-end, or both of the 3′ and 5′-ends, being present in the same or different strand.

In some aspects, a polynucleic acid molecule comprises a sense strand and an antisense strand, in which the antisense strand comprises about 1 to about 25 or more, for example, about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the sense strand; and the antisense strand comprises about 1 to about 25 or more, for example about 1, 2, 3, 4, 5, 6, 7, 8, 9, 10, 11, 12, 13, 14, 15, 16, 17, 18, 19, 20, or more phosphorothioate internucleotide linkages, and/or one or more (e.g., about 1, 2, 3, 4, 5, 6, 7, 8, 9, 10 or more) 2′-deoxy, 2′-O-methyl, 2′-deoxy-2′-fluoro, and/or one or more (e.g., about 1, 2, 3, 4, 5, 6, 7, 8, 9, 10 or more) universal base modified nucleotides, and optionally a terminal cap molecule at the 3′-end, the 5′-end, or both of the 3′- and 5′-ends of the antisense strand. In other embodiments, one or more, for example about 1, 2, 3, 4, 5, 6, 7, 8, 9, 10 or more pyrimidine nucleotides of the sense and/or antisense strand are chemically-modified with 2′-deoxy, 2′-O-methyl and/or 2′-deoxy-2′-fluoro nucleotides, with or without about 1 to about 5, for example about 1, 2, 3, 4, 5 or more phosphorothioate internucleotide linkages and/or a terminal cap molecule at the 3′-end, the 5′-end, or both of the 3′- and 5′-ends, being present in the same or different strand.

In some aspects, a polynucleic acid molecule described herein is a chemically-modified short interfering nucleic acid molecule having about 1 to about 25, for example, about 1, 2, 3, 4, 5, 6, 7, 8, 9, 10, 11, 12, 13, 14, 15, 16, 17, 18, 19, 20 or more phosphorothioate internucleotide linkages in each strand of the polynucleic acid molecule. In some aspects, a polynucleic acid molecule comprises a sense strand and an antisense strand, and the antisense strand comprises a phosphate backbone modification at the 3′ end of the antisense strand. Alternatively and/or additionally, a polynucleic acid molecule comprises a sense strand and an antisense strand, and the sense strand comprises a phosphate backbone modification at the 5′ end of the antisense strand. In some instances, the phosphate backbone modification is a phosphorothioate. In some aspects, the sense or antisense strand has three consecutive nucleosides that are coupled via two phosphorothioate backbone.

In another embodiment, a polynucleic acid molecule described herein comprises 2′-5′ internucleotide linkages. In some instances, the 2′-5′ internucleotide linkage(s) is at the 3′-end, the 5′-end, or both of the 3′- and 5′-ends of one or both sequence strands. In addition instances, the 2′-5′ internucleotide linkage(s) is present at various other positions within one or both sequence strands, for example, about 1, 2, 3, 4, 5, 6, 7, 8, 9, 10, or more including every internucleotide linkage of a pyrimidine nucleotide in one or both strands of the polynucleic acid molecule comprise a 2′-5′ internucleotide linkage, or about 1, 2, 3, 4, 5, 6, 7, 8, 9, 10, or more including every internucleotide linkage of a purine nucleotide in one or both strands of the polynucleic acid molecule comprise a 2′-5′ internucleotide linkage.

In some aspects, a polynucleic acid molecule is a single stranded polynucleic acid molecule that mediates RNAi activity in a cell or reconstituted in vitro system, wherein the polynucleic acid molecule comprises a single stranded polynucleotide having complementarity to a target nucleic acid sequence, and wherein one or more pyrimidine nucleotides present in the polynucleic acid are 2′-deoxy-2′-fluoro pyrimidine nucleotides (e.g., wherein all pyrimidine nucleotides are 2′-deoxy-2′-fluoro pyrimidine nucleotides or alternately a plurality of pyrimidine nucleotides are 2′-deoxy-2′-fluoro pyrimidine nucleotides), and wherein any purine nucleotides present in the polynucleic acid are 2′-deoxy purine nucleotides (e.g., wherein all purine nucleotides are 2′-deoxy purine nucleotides or alternately a plurality of purine nucleotides are 2′-deoxy purine nucleotides), and a terminal cap modification, that is optionally present at the 3′-end, the 5′-end, or both of the 3′ and 5′-ends of the antisense sequence, the polynucleic acid molecule optionally further comprising about 1 to about 4 (e.g., about 1, 2, 3, or 4) terminal 2′-deoxynucleotides at the 3′-end of the polynucleic acid molecule, wherein the terminal nucleotides further comprise one or more (e.g., 1, 2, 3, or 4) phosphorothioate internucleotide linkages, and wherein the polynucleic acid molecule optionally further comprises a terminal phosphate group, such as a 5′-terminal phosphate group.

In some cases, one or more of the artificial nucleotide analogues described herein are resistant toward nucleases such as for example ribonuclease such as RNase H, deoxyribonuclease such as DNase, or exonuclease such as 5′-3′ exonuclease and 3′-5′ exonuclease when compared to natural polynucleic acid molecules. In some instances, artificial nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, 2′-fluoro N3-P5′-phosphoramidites, or combinations thereof are resistant toward nucleases such as for example ribonuclease such as RNase H, deoxyribonuclease such as DNase, or exonuclease such as 5′-3′ exonuclease and 3′-5′ exonuclease. In some instances, 2′-O-methyl modified polynucleic acid molecule is nuclease resistance (e.g., RNase H, DNase, 5′-3′ exonuclease or 3′-5′ exonuclease resistance). In some instances, 2′O-methoxyethyl (2′-O-MOE) modified polynucleic acid molecule is nuclease resistance (e.g., RNase H, DNase, 5′-3′ exonuclease or 3′-5′ exonuclease resistance). In some instances, 2′-O-aminopropyl modified polynucleic acid molecule is nuclease resistance (e.g., RNase H, DNase, 5′-3′ exonuclease or 3′-5′ exonuclease resistance). In some instances, 2′-deoxy modified polynucleic acid molecule is nuclease resistance (e.g., RNase H, DNase, 5′-3′ exonuclease or 3′-5′ exonuclease resistance). In some instances, 2′-deoxy-2′-fluoro modified polynucleic acid molecule is nuclease resistance (e.g., RNase H, DNase, 5′-3′ exonuclease or 3′-5′ exonuclease resistance). In some instances, 2′-O-aminopropyl (2′-O-AP) modified polynucleic acid molecule is nuclease resistance (e.g., RNase H, DNase, 5′-3′ exonuclease or 3′-5′ exonuclease resistance). In some instances, 2′-O-dimethylaminoethyl (2′-O-DMAOE) modified polynucleic acid molecule is nuclease resistance (e.g., RNase H, DNase, 5′-3′ exonuclease or 3′-5′ exonuclease resistance). In some instances, 2′-O-dimethylaminopropyl (2′-O-DMAP) modified polynucleic acid molecule is nuclease resistance (e.g., RNase H, DNase, 5′-3′ exonuclease or 3′-5′ exonuclease resistance). In some instances, 2′-O-dimethylaminoethyloxyethyl (2′-O-DMAEOE) modified polynucleic acid molecule is nuclease resistance (e.g., RNase H, DNase, 5′-3′ exonuclease or 3′-5′ exonuclease resistance). In some instances, 2′-O—N-methylacetamido (2′-O-NMA) modified polynucleic acid molecule is nuclease resistance (e.g., RNase H, DNase, 5′-3′ exonuclease or 3′-5′ exonuclease resistance). In some instances, LNA modified polynucleic acid molecule is nuclease resistance (e.g., RNase H, DNase, 5′-3′ exonuclease or 3′-5′ exonuclease resistance). In some instances, ENA modified polynucleic acid molecule is nuclease resistance (e.g., RNase H, DNase, 5′-3′ exonuclease or 3′-5′ exonuclease resistance). In some instances, HNA modified polynucleic acid molecule is nuclease resistance (e.g., RNase H, DNase, 5′-3′ exonuclease or 3′-5′ exonuclease resistance). In some instances, morpholinos is nuclease resistance (e.g., RNase H, DNase, 5′-3′ exonuclease or 3′-5′ exonuclease resistance). In some instances, PNA modified polynucleic acid molecule is resistant to nucleases (e.g., RNase H, DNase, 5′-3′ exonuclease or 3′-5′ exonuclease resistance). In some instances, methylphosphonate nucleotides modified polynucleic acid molecule is nuclease resistance (e.g., RNase H, DNase, 5′-3′ exonuclease or 3′-5′ exonuclease resistance). In some instances, thiolphosphonate nucleotides modified polynucleic acid molecule is nuclease resistance (e.g., RNase H, DNase, 5′-3′ exonuclease or 3′-5′ exonuclease resistance). In some instances, polynucleic acid molecule comprising 2′-fluoro N3-P5′-phosphoramidites is nuclease resistance (e.g., RNase H, DNase, 5′-3′ exonuclease or 3′-5′ exonuclease resistance). In some instances, the 5′ conjugates described herein inhibit 5′-3′ exonucleolytic cleavage. In some instances, the 3′ conjugates described herein inhibit 3′-5′ exonucleolytic cleavage.

In some aspects, one or more of the artificial nucleotide analogues described herein have increased binding affinity toward their mRNA target relative to an equivalent natural polynucleic acid molecule. The one or more of the artificial nucleotide analogues comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified, LNA, ENA, PNA, HNA, morpholino, methylphosphonate nucleotides, thiolphosphonate nucleotides, or 2′-fluoro N3-P5′-phosphoramidites have increased binding affinity toward their mRNA target relative to an equivalent natural polynucleic acid molecule. In some instances, 2′-O-methyl modified polynucleic acid molecule has increased binding affinity toward their mRNA target relative to an equivalent natural polynucleic acid molecule. In some instances, 2′-O-methoxyethyl (2′-O-MOE) modified polynucleic acid molecule has increased binding affinity toward their mRNA target relative to an equivalent natural polynucleic acid molecule. In some instances, 2′-O-aminopropyl modified polynucleic acid molecule has increased binding affinity toward their mRNA target relative to an equivalent natural polynucleic acid molecule. In some instances, 2′-deoxy modified polynucleic acid molecule has increased binding affinity toward their mRNA target relative to an equivalent natural polynucleic acid molecule. In some instances, 2′-deoxy-2′-fluoro modified polynucleic acid molecule has increased binding affinity toward their mRNA target relative to an equivalent natural polynucleic acid molecule. In some instances, 2′-O-aminopropyl (2′-O-AP) modified polynucleic acid molecule has increased binding affinity toward their mRNA target relative to an equivalent natural polynucleic acid molecule. In some instances, 2′-O-dimethylaminoethyl (2′-O-DMAOE) modified polynucleic acid molecule has increased binding affinity toward their mRNA target relative to an equivalent natural polynucleic acid molecule. In some instances, 2′-O-dimethylaminopropyl (2′-O-DMAP) modified polynucleic acid molecule has increased binding affinity toward their mRNA target relative to an equivalent natural polynucleic acid molecule. In some instances, 2′-O-dimethylaminoethyloxyethyl (2′-O-DMAEOE) modified polynucleic acid molecule has increased binding affinity toward their mRNA target relative to an equivalent natural polynucleic acid molecule. In some instances, 2′-O—N-methylacetamido (2′-O-NMA) modified polynucleic acid molecule has increased binding affinity toward their mRNA target relative to an equivalent natural polynucleic acid molecule. In some instances, LNA modified polynucleic acid molecule has increased binding affinity toward their mRNA target relative to an equivalent natural polynucleic acid molecule. In some instances, ENA modified polynucleic acid molecule has increased binding affinity toward their mRNA target relative to an equivalent natural polynucleic acid molecule. In some instances, PNA modified polynucleic acid molecule has increased binding affinity toward their mRNA target relative to an equivalent natural polynucleic acid molecule. In some instances, HNA modified polynucleic acid molecule has increased binding affinity toward their mRNA target relative to an equivalent natural polynucleic acid molecule. In some instances, morpholino modified polynucleic acid molecule has increased binding affinity toward their mRNA target relative to an equivalent natural polynucleic acid molecule. In some instances, methylphosphonate nucleotides modified polynucleic acid molecule has increased binding affinity toward their mRNA target relative to an equivalent natural polynucleic acid molecule. In some instances, thiolphosphonate nucleotides modified polynucleic acid molecule has increased binding affinity toward their mRNA target relative to an equivalent natural polynucleic acid molecule. In some instances, polynucleic acid molecule comprising 2′-fluoro N3-P5′-phosphoramidites has increased binding affinity toward their mRNA target relative to an equivalent natural polynucleic acid molecule. In some cases, the increased affinity is illustrated with a lower Kd, a higher melt temperature (Tm), or a combination thereof.

In some aspects, a polynucleic acid molecule described herein is a chirally pure (or stereo pure) polynucleic acid molecule, or a polynucleic acid molecule comprising a single enantiomer. In some instances, the polynucleic acid molecule comprises L-nucleotide. In some instances, the polynucleic acid molecule comprises D-nucleotides. In some instance, a polynucleic acid molecule composition comprises less than 30%, 25%, 20%, 15%, 10%, 5%, 4%, 3%, 2%, 1%, or less of its mirror enantiomer. In some cases, a polynucleic acid molecule composition comprises less than 30%, 25%, 20%, 15%, 10%, 5%, 4%, 3%, 2%, 1%, or less of a racemic mixture. In some instances, the polynucleic acid molecule is a polynucleic acid molecule described in: U.S. Patent Publication Nos: 2014/194610 and 2015/211006; and PCT Publication No: WO2015107425.

In some aspects, a polynucleic acid molecule described herein is further modified to include an aptamer conjugating moiety. In some instances, the aptamer conjugating moiety is a DNA aptamer conjugating moiety. In some instances, the aptamer conjugating moiety is Alphamer (Centauri Therapeutics), which comprises an aptamer portion that recognizes a specific cell-surface target and a portion that presents a specific epitope for attaching to circulating antibodies. In some instance, a polynucleic acid molecule described herein is further modified to include an aptamer conjugating moiety as described in: U.S. Pat. Nos. 8,604,184, 8,591,910, and 7,850,975.

In additional embodiments, a polynucleic acid molecule described herein is modified to increase its stability. In some embodiments, the polynucleic acid molecule is RNA (e.g., siRNA). In some instances, the polynucleic acid molecule is modified by one or more of the modifications described above to increase its stability. In some cases, the polynucleic acid molecule is modified at the 2′ hydroxyl position, such as by 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modification or by a locked or bridged ribose conformation (e.g., LNA or ENA). In some cases, the polynucleic acid molecule is modified by 2′-O-methyl and/or 2′-O-methoxyethyl ribose. In some cases, the polynucleic acid molecule also includes morpholinos, PNAs, HNA, methylphosphonate nucleotides, thiolphosphonate nucleotides, and/or 2′-fluoro N3-P5′-phosphoramidites to increase its stability. In some instances, the polynucleic acid molecule is a chirally pure (or stereo pure) polynucleic acid molecule. In some instances, the chirally pure (or stereo pure) polynucleic acid molecule is modified to increase its stability. Suitable modifications to the RNA to increase stability for delivery will be apparent to the skilled person.

In some instances, the polynucleic acid molecule is a double-stranded polynucleotide molecule comprising self-complementary sense and antisense regions, wherein the antisense region comprises nucleotide sequence that is partially or fully complementary to nucleotide sequence in a target nucleic acid molecule or a portion thereof and the sense region having nucleotide sequence partially or fully corresponding to the target nucleic acid sequence or a portion thereof. In some instances, the polynucleic acid molecule is assembled from two separate polynucleotides, where one strand is the sense strand and the other is the antisense strand, wherein the antisense and sense strands are self-complementary (e.g., each strand comprises nucleotide sequence that is complementary to nucleotide sequence in the other strand; such as where the antisense strand and sense strand form a duplex or double stranded structure, for example wherein the double stranded region is about 19, 20, 21, 22, 23, or more base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the polynucleic acid molecule is assembled from a single oligonucleotide, where the self-complementary sense and antisense regions of the polynucleic acid molecule are linked by means of a nucleic acid based or non-nucleic acid-based linker(s).

In some cases, the polynucleic acid molecule is a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. In other cases, the polynucleic acid molecule is a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide is processed either in vivo or in vitro to generate an active polynucleic acid molecule capable of mediating RNAi. In additional cases, the polynucleic acid molecule also comprises a single-stranded polynucleotide having nucleotide sequence complementary to nucleotide sequence in a target nucleic acid molecule or a portion thereof (for example, where such polynucleic acid molecule does not require the presence within the polynucleic acid molecule of nucleotide sequence corresponding to the target nucleic acid sequence or a portion thereof), wherein the single stranded polynucleotide further comprises a terminal phosphate group, such as a 5′-phosphate (see for example Martinez et al., 2002, Cell, 110, 563-574 and Schwarz et al., 2002, Molecular Cell, 10, 537-568), or 5′,3′-diphosphate.

In some instances, an asymmetric hairpin is a linear polynucleic acid molecule comprising an antisense region, a loop portion that comprises nucleotides or non-nucleotides, and a sense region that comprises fewer nucleotides than the antisense region to the extent that the sense region has enough complimentary nucleotides to base pair with the antisense region and form a duplex with loop. For example, an asymmetric hairpin polynucleic acid molecule comprises an antisense region having length sufficient to mediate RNAi in a cell or in vitro system (e.g. about 19 to about 22 nucleotides) and a loop region comprising about 4 to about 8 nucleotides, and a sense region having about 3 to about 18 nucleotides that are complementary to the antisense region. In some cases, the asymmetric hairpin polynucleic acid molecule also comprises a 5′-terminal phosphate group that is chemically modified. In additional cases, the loop portion of the asymmetric hairpin polynucleic acid molecule comprises nucleotides, non-nucleotides, linker molecules, or conjugate molecules.

In some aspects, an asymmetric duplex is a polynucleic acid molecule having two separate strands comprising a sense region and an antisense region, wherein the sense region comprises fewer nucleotides than the antisense region to the extent that the sense region has enough complimentary nucleotides to base pair with the antisense region and form a duplex. For example, an asymmetric duplex polynucleic acid molecule comprises an antisense region having length sufficient to mediate RNAi in a cell or in vitro system (e.g., about 19 to about 22 nucleotides) and a sense region having about 3 to about 18 nucleotides that are complementary to the antisense region.

In some cases, a universal base refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

### Target Genes

In some instances, the polynucleic acid molecule targets an incorrectly processed pre-mRNA transcript which results in a disease or disorder not limited to a neuromuscular disease, a genetic disease, cancer, a hereditary disease, or a cardiovascular disease.

In some instances, a polynucleic acid molecule targets an exon that is mutated in a gene that causes a disease or disorder. Exemplary diseases or disorders include, but are not limited to, familial dysautonomia (FD), spinal muscular atrophy (SMA), medium-chain acyl-CoA dehydrogenase (MCAD) deficiency, Hutchinson-Gilford progeria syndrome (HGPS), myotonic dystrophy type I (DM1), myotonic dystrophy type II (DM2), autosomal dominant retinitis pigmentosa (RP), Duchenne muscular dystrophy (DMD), microcephalic steodysplastic primordial dwarfism type 1 (MOPD1) (Taybi-Linder syndrome (TALS), frontotemporal dementia with parkinsonism-17 (FTDP-17), Facioscapulohumeral muscular dystrophy (FSHD) Fukuyama congenital muscular dystrophy (FCMD), amyotrophic lateral sclerosis (ALS), hypercholesterolemia, and cystic fibrosis (CF). Exemplary genes that are involved in the disease or disorder include, but are not limited to, DUX4, IKBKAP, SMN2, MCAD, LMNA, DMPK, ZNF9, MAPT, FKTN, TDP-43, LDLR, CFTR, DMD, PAH, MSTN, K-Ras, GYS1, PLN, PRKAG2, and/or CNBP. In some embodiments, the gene is DMD, PAH, MSTN, HPRT1, SSB, or K-Ras.

In some embodiments, a polynucleic acid molecule hybridizes to a target sequence of an atrophy-related gene (also referred to as an atrogene). In some instances, a polynucleic acid molecule described herein hybridizes to a target sequence of an ubiquitin ligase (e.g., an E3 ubiquitin ligase or a mitochondrial ubiquitin ligase). In some instances, a polynucleic acid molecule described herein hybridizes to a target sequence of a Forkhead box transcription factor. In some instances, a polynucleic acid molecule described herein hybridizes to a target sequence of a growth factor. In some instances, a polynucleic acid molecule described herein hybridizes to a target sequence of a deubiquitinating enzyme.

In some embodiments, a polynucleic acid molecule described herein hybridizes to a target sequence of HPRT, FBXO32, TRIM63, TRAF6, FBXO30, FBXO40, NEDD4, TRIM32, MUL1, STUB1, FOXO1, FOXO3, MSTN, USP14, USP19, DDIT4, CTSL2, TGIF, MYOG, HDAC2, HDAC3, MT1L, MT1B, SSB, DMPK, DMD, DUX4, GYS1, PLN, PRKAG2, and/or CNBP.

In some embodiments, a polynucleic acid molecule conjugate or a lipid modified polynucleic acid molecule conjugate described herein hybridizes different target sequences. In some embodiments, a polynucleic acid molecule conjugate or a lipid modified polynucleic acid molecule conjugate described herein target mRNAs of different genes.

In some embodiments, the polynucleic acid molecule comprises a sequence having at least 50%, 55%, 60%, 65%, 70%, 75%, 80%, 85%, 90%, 95%, 96%, 97%, 98%, 99%, or 100% sequence identity to a sequence listed in Tables 5, 8, 12, 16, 19, 22, 25, 28, 32, 40, 43, 46, 49, 52, 55, 58, 61, 64, 67, 70, 73, 76, 77, 78, 80, and 82.

In some embodiments, the polynucleic acid molecule is a polynucleic acid disclosed in PCT/US2017/025608, PCT/US2018/012672, PCT/US2018/052289, PCT/US2018/064359, PCT/US2020/029731, PCT/US2021/022214, PCT/US2021/024303, PCT/US2022/043705, PCT/US2023/017574, PCT/US2024/035667, PCT/US2024/036259, PCT/US2024/049484, PCT/US2025/022623, and PCT/US2025/022622.

### Polynucleic Acid Molecule Synthesis

In some aspects, a polynucleic acid molecule described herein is constructed using chemical synthesis and/or enzymatic ligation reactions using procedures known in the art. For example, a polynucleic acid molecule is chemically synthesized using naturally occurring nucleotides or variously modified nucleotides designed to increase the biological stability of the molecules or to increase the physical stability of the duplex formed between the polynucleic acid molecule and target nucleic acids. Exemplary methods include those described in: U.S. Pat. Nos. 5,142,047; 5,185,444; 5,889,136; 6,008,400; and 6,111,086; PCT Publication No. WO2009099942; or European Publication NO. 1579015. Additional exemplary methods include those described in: Griffey et al., “2′-O-aminopropyl ribonucleotides: a zwitterionic modification that enhances the exonuclease resistance and biological activity of antisense oligonucleotides,” J. Med. Chem. 39(26):5100-5109 (1997)); Obika, et al. “Synthesis of 2′-0,4′-C-methyleneuridine and -cytidine. Novel bicyclic nucleosides having a fixed C₃, -endo sugar puckering”. Tetrahedron Letters 38 (50): 8735 (1997); Koizumi, M. “ENA oligonucleotides as therapeutics”. Current opinion in molecular therapeutics 8 (2): 144-149 (2006); and Abramova et al., “Novel oligonucleotide analogues based on morpholino nucleoside subunits-antisense technologies: new chemical possibilities,” Indian Journal of Chemistry 48B:1721-1726 (2009). Alternatively, the polynucleic acid molecule is produced biologically using an expression vector into which a polynucleic acid molecule has been subcloned in an antisense orientation (i.e., RNA transcribed from the inserted polynucleic acid molecule will be of an antisense orientation to a target polynucleic acid molecule of interest).

In some aspects, a polynucleic acid molecule is synthesized via a tandem synthesis methodology, wherein both strands are synthesized as a single contiguous oligonucleotide fragment or strand separated by a cleavable linker which is subsequently cleaved to provide separate fragments or strands that hybridize and permit purification of the duplex.

In some instances, a polynucleic acid molecule is also assembled from two distinct nucleic acid strands or fragments wherein one fragment includes the sense region and the second fragment includes the antisense region of the molecule.

Additional modification methods for incorporating, for example, sugar, base and phosphate modifications include: Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010. Such publications describe general methods and strategies to determine the location of incorporation of sugar, base and/or phosphate modifications and the like into nucleic acid molecules without modulating catalysis.

In some instances, while chemical modification of the polynucleic acid molecule internucleotide linkages with phosphorothioate, phosphorodithioate, and/or 5′-methylphosphonate linkages improves stability, excessive modifications sometimes cause toxicity or decreased activity. Therefore, when designing nucleic acid molecules, the amount of these internucleotide linkages in some cases is minimized. In such cases, the reduction in the concentration of these linkages lowers toxicity, increases efficacy and higher specificity of these molecules.

### Conjugation Chemistry

In some embodiments, a lipid modified polynucleic acid molecule or polynucleic acid molecule is conjugated to a binding moiety. In some instances, the binding moiety comprises amino acids, peptides, polypeptides, proteins, antibodies, antigens, toxins, hormones, lipids, nucleotides, nucleosides, sugars, carbohydrates, polymers such as polyethylene glycol and polypropylene glycol, as well as analogs or derivatives of all of these classes of substances. Additional examples of binding moiety also include steroids, such as cholesterol, phospholipids, di- and triacylglycerols, fatty acids, hydrocarbons (e.g., saturated, unsaturated, or contains substitutions), enzyme substrates, biotin, digoxigenin, and polysaccharides. In some instances, the binding moiety is an antibody or antigen binding fragment thereof.

In some embodiments, the lipid modified polynucleic acid molecule or polynucleic acid molecule is conjugated to the binding moiety by a chemical ligation process. In some instances, the polynucleic acid molecule or polynucleic acid molecule is conjugated to the binding moiety by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910. In some embodiments, the lipid modified polynucleic acid molecule or polynucleic acid molecule is conjugated to the binding moiety either site-specifically or non-specifically via native ligation chemistry.

In some instances, the lipid modified polynucleic acid molecule or polynucleic acid molecule is conjugated to the binding moiety by a site-directed method utilizing a “traceless” coupling technology (Philochem). In some instances, the “traceless” coupling technology utilizes an N-terminal 1,2-aminothiol group on the binding moiety which is then conjugated with a polynucleic acid molecule or polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the lipid modified polynucleic acid molecule or polynucleic acid molecule is conjugated to the binding moiety by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivative conjugating moiety to form an oxime bond. (see Axup et al., “Synthesis of site-specific antibody-drug conjugates using unnatural amino acids,” PNAS 109(40): 16101-16106 (2012)).

In some instances, the lipid modified polynucleic acid molecule or polynucleic acid molecule is conjugated to the binding moiety by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some instances, the enzyme-catalyzed process comprises microbial transglutaminase (mTG). In some cases, the lipid modified polynucleic acid molecule or polynucleic acid molecule is conjugated to the binding moiety utilizing a microbial transglutaminze catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobaraensis. (see Strop et al., “Location matters: site of conjugation modulates stability and pharmacokinetics of antibody drug conjugates,” Chemistry and Biology 20(2) 161-167 (2013)).

In some instances, the lipid modified polynucleic acid molecule or polynucleic acid molecule is conjugated to the binding moiety by a method as described in PCT Publication No. WO2014/140317, which utilizes a sequence-specific transpeptidase.

In some instances, the lipid modified polynucleic acid molecule or polynucleic acid molecule is conjugated to the binding moiety by a method as described in U.S. Patent Publication Nos. 2015/0105539 and 2015/0105540.

In some instances, the lipid modified polynucleic acid molecule or polynucleic acid molecule is conjugated to thiol reactive group on the binding moiety (e.g., traut's alkyl thiols, substitute alkyl thiols). In some instances, the lipid modified polynucleic acid molecule or polynucleic acid molecule is conjugated to the binding moiety via a disulfide linkage to a pyridyl (pyr) moiety. In some instances, the lipid modified polynucleic acid molecule or polynucleic acid molecule is conjugated to the binding moiety by squaric acid ester-based synthesis.

### Binding Moiety

In some embodiments, the binding moiety is a polypeptide. In some instances, the polypeptide is an antibody or its fragment thereof. In some cases, the fragment is a binding fragment. In some instances, the antibody or antigen binding fragment thereof comprises a humanized antibody or antigen binding fragment thereof, murine antibody or antigen binding fragment thereof, chimeric antibody or antigen binding fragment thereof, monoclonal antibody or antigen binding fragment thereof, monovalent Fab′, divalent Fab2, F(ab)′₃ fragments, single-chain variable fragment (scFv), bis-scFv, (scFv)₂, single-arm antibody, diabody, minibody, nanobody, triabody, tetrabody, disulfide stabilized Fv protein (dsFv), single-domain antibody (sdAb), Ig NAR, camelid antibody or antigen binding fragment thereof, bispecific antibody or biding fragment thereof, or a chemically modified derivative thereof. In some embodiments, the antibody or antigen binding fragment thereof is a Fab fragment. In some embodiments, the antibody or antigen binding fragment thereof is a VHH. In some embodiments, the antibody or antigen binding fragment thereof is a Fab-Fc fusion antibody. In some embodiments, the antibody or antigen binding fragment thereof is a VHH-Fc fusion antibody.

In some instances, the binding moiety is an antibody or antigen binding fragment thereof. In some instances, the binding moiety is a humanized antibody or antigen binding fragment thereof, murine antibody or antigen binding fragment thereof, chimeric antibody or antigen binding fragment thereof, monoclonal antibody or antigen binding fragment thereof, monovalent Fab′, divalent Fab2, F(ab)′₃ fragments, single-chain variable fragment (scFv), bis-scFv, (scFv)₂, diabody, minibody, nanobody, triabody, tetrabody, disulfide stabilized Fv protein (“dsFv”), single-domain antibody (sdAb), Ig NAR, camelid antibody or antigen binding fragment thereof, bispecific antibody or antigen binding fragment thereof, or a chemically modified derivative thereof. In some instances, the binding moiety is a humanized antibody or antigen binding fragment thereof. In some instances, the binding moiety is a murine antibody or antigen binding fragment thereof. In some instances, the binding moiety is a chimeric antibody or antigen binding fragment thereof. In some instances, the binding moiety is a monoclonal antibody or antigen binding fragment thereof. In some instances, the binding moiety is a monovalent Fab′. In some instances, the binding moiety is a divalent Fab₂. In some instances, A is a single-chain variable fragment (scFv).

In some embodiments, the binding moiety is a bispecific antibody or antigen binding fragment thereof. In some instances, the bispecific antibody is a trifunctional antibody or a bispecific mini-antibody. In some cases, the bispecific antibody is a trifunctional antibody. In some instances, the trifunctional antibody is a full length monoclonal antibody comprising binding sites for two different antigens. Exemplary trifunctional antibodies include catumaxomab (which targets EpCAM and CD3; Fresenius Biotech/Trion Pharma), ertumaxomab (targets HER2/neu/CD3; Fresenius Biotech/Trion Pharma), lymphomun FBTA05 (targets CD20/CD3; Fresenius Biotech/Trion Pharma), RG7221 (RO5520985; targets Angiopoietin 2/VEGF; Roche), RG7597 (targets Her1/Her3; Genentech/Roche), MM141 (targets IGF1R/Her3; Merrimack), ABT122 (targets TNFα/IL17; Abbvie), ABT981 (targets IL1α/IL1β; Abbott), LY3164530 (targets Her1/cMET; Eli Lilly), and TRBS07 (Ektomab; targets GD2/CD3; Trion Research Gmbh). Additional exemplary trifunctional antibodies include mAb² from F-star Biotechnology Ltd. In some instances, A is a bispecific trifunctional antibody. In some embodiments, A is a bispecific trifunctional antibody selected from: catumaxomab (which targets EpCAM and CD3; Fresenius Biotech/Trion Pharma), ertumaxomab (targets HER2/neu/CD3; Fresenius Biotech/Trion Pharma), lymphomun FBTA05 (targets CD20/CD3; Fresenius Biotech/Trion Pharma), RG7221 (RO5520985; targets Angiopoietin 2/VEGF; Roche), RG7597 (targets Her1/Her3; Genentech/Roche), MM141 (targets IGF1R/Her3; Merrimack), ABT122 (targets TNFα/IL17; Abbvie), ABT981 (targets IL1α/IL1β; Abbott), LY3164530 (targets Her1/cMET; Eli Lilly), TRBS07 (Ektomab; targets GD2/CD3; Trion Research Gmbh), and a mAb² from F-star Biotechnology Ltd.

In some cases, the bispecific antibody is a bispecific mini-antibody. In some instances, the bispecific mini-antibody comprises divalent Fab2, F(ab)′₃ fragments, bis-scFv, (scFv)₂, diabody, minibody, triabody, tetrabody or a bi-specific T-cell engager (BiTE). In some embodiments, the bi-specific T-cell engager is a fusion protein that contains two single-chain variable fragments (scFvs) in which the two scFvs target epitopes of two different antigens. Exemplary bispecific mini-antibodies include, but are not limited to, DART (dual-affinity re-targeting platform; MacroGenics), blinatumomab (MT103 or AMG103; which targets CD19/CD3; Micromet), MT111 (targets CEA/CD3; Micromet/Amegen), MT112 (BAY2010112; targets PSMA/CD3; Micromet/Bayer), MT110 (AMG 110; targets EPCAM/CD3; Amgen/Micromet), MGD006 (targets CD123/CD3; MacroGenics), MGD007 (targets GPA33/CD3; MacroGenics), BI1034020 (targets two different epitopes on β-amyloid; Ablynx), ALX0761 (targets IL17A/IL17F; Ablynx), TF2 (targets CEA/hepten; Immunomedics), IL-17/IL-34 biAb (BMS), AFM13 (targets CD30/CD16; Affimed), AFM11 (targets CD19/CD3; Affimed), and domain antibodies (dAbs from Domantis/GSK).

In some embodiments, the binding moiety is a trispecific antibody. In some instances, the trispecific antibody comprises F(ab)′₃ fragments or a triabody. In some instances, the binding moiety is a trispecific F(ab)′₃ fragment. In some cases, the binding moiety is a triabody. In some embodiments, the binding moiety is a trispecific antibody as described in Dimas, et al., “Development of a trispecific antibody designed to simultaneously and efficiently target three different antigens on tumor cells,” Mol. Pharmaceutics, 12(9): 3490-3501 (2015).

In some embodiments, the binding moiety is an antibody or antigen binding fragment thereof that recognizes a cell surface protein. In some instances, the cell surface protein is an antigen expressed by a cancerous cell. Exemplary cancer antigens include, but are not limited to, alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAP1B, SPAP1C), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Rα, Immunoglobulin superfamily receptor translocation associated 2 (IRTA2), L6, Lewis Y, Lewis X, MAGE-1, MAGE-2, MAGE-3, MAGE 4, MART1, mesothelin, MDP, MPF (SMR, MSLN), MCP1 (CCL2), macrophage inhibitory factor (MIF), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, anti-transferrin receptor, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, FLJ20041, TRPM4, TRPM4B, transient receptor potential cation channel, subfamily M, member 4) and the like.

In some instances, the binding moiety is an antibody or antigen binding fragment thereof that recognizes a CD cell surface marker. In some instances, the binding moiety is an antibody or antigen binding fragment thereof that recognizes CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD71, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), or a combination thereof.

In some instances, the antibody or antigen binding fragment thereof is an anti-transferrin receptor (anti-CD71) antibody, the anti-transferrin antibody specifically binds to a transferrin receptor (TfR), preferably, specifically binds to transferrin receptor 1 (TfR1), or more preferably, specifically binds to human transferrin receptor 1 (TfR1) (or human CD71).

In some embodiments, the antibody or antigen binding fragment thereof is an antibody or antigen binding fragment thereof. WO 2020/132584, WO 2022/020105, WO 2021/154476, WO 2021/154477, WO2023/283620, US2023/0088865, U.S. Pat. Nos. 11,672,872, 11,839,660, 11,969,475, WO2024/006976, WO2025/085352, WO2024/036096, WO2024/026474, and US2025/235549, each of which is incorporated herein by reference in its entirety.

In some embodiments, the binding moiety is a multicyclic peptide. In some embodiments, the binding moiety is a bicyclic or tricyclic peptide. In some embodiments, the binding moiety is a bicyclic or tricyclic peptide specifically binds to a transferrin receptor (TfR), preferably, specifically binds to transferrin receptor 1 (TfR1), or more preferably, specifically binds to human transferrin receptor 1 (TfR1) (or human CD71).

In some embodiments, the binding moiety is conjugated to a polynucleic acid molecule. In some instances, the binding moiety is conjugated to a polynucleic acid molecule via a lysine residue or a cysteine residue, in a non-site specific manner. In some instances, the binding moiety is conjugated to a polynucleic acid molecule via a lysine residue in a non-site specific manner. In some cases, the binding moiety is conjugated to a polynucleic acid molecule via a cysteine residue in a non-site specific manner.

In some embodiments, the binding moiety is conjugated to a polynucleic acid molecule in a site-specific manner. In some instances, the binding moiety is conjugated to a polynucleic acid molecule through a lysine residue, a cysteine residue, at the 5′-terminus, at the 3′-terminus, an unnatural amino acid, or an enzyme-modified or enzyme-catalyzed residue, via a site-specific manner. In some instances, the binding moiety is conjugated to a polynucleic acid molecule through a lysine residue via a site-specific manner. In some instances, the binding moiety is conjugated to a polynucleic acid molecule through a cysteine residue via a site-specific manner. In some instances, the binding moiety is conjugated to a polynucleic acid molecule at the 5′-terminus via a site-specific manner. In some instances, the binding moiety is conjugated to a polynucleic acid molecule at the 3′-terminus via a site-specific manner. In some instances, the binding moiety is conjugated to a polynucleic acid molecule through an unnatural amino acid via a site-specific manner. In some instances, the binding moiety is conjugated to a polynucleic acid molecule through an enzyme-modified or enzyme-catalyzed residue via a site-specific manner.

In some embodiments, one or more polynucleic acid molecule is conjugated to a binding moiety. In some instances, about 1, 2, 3, 4, 5, 6, 7, 8, 9, 10, 11, 12, 13, 14, 15, 16, or more polynucleic acid molecules are conjugated to one binding moiety. In some instances, about 1 polynucleic acid molecule is conjugated to one binding moiety. In some instances, about 2 polynucleic acid molecules are conjugated to one binding moiety. In some instances, about 3 polynucleic acid molecules are conjugated to one binding moiety. In some instances, about 4 polynucleic acid molecules are conjugated to one binding moiety. In some instances, about 5 polynucleic acid molecules are conjugated to one binding moiety. In some instances, about 6 polynucleic acid molecules are conjugated to one binding moiety. In some instances, about 7 polynucleic acid molecules are conjugated to one binding moiety. In some instances, about 8 polynucleic acid molecules are conjugated to one binding moiety. In some instances, about 9 polynucleic acid molecules are conjugated to one binding moiety. In some instances, about 10 polynucleic acid molecules are conjugated to one binding moiety. In some instances, about 11 polynucleic acid molecules are conjugated to one binding moiety. In some instances, about 12 polynucleic acid molecules are conjugated to one binding moiety. In some instances, about 13 polynucleic acid molecules are conjugated to one binding moiety. In some instances, about 14 polynucleic acid molecules are conjugated to one binding moiety. In some instances, about 15 polynucleic acid molecules are conjugated to one binding moiety. In some instances, about 16 polynucleic acid molecules are conjugated to one binding moiety. In some cases, the one or more polynucleic acid molecules are the same. In other cases, the one or more polynucleic acid molecules are different.

In some embodiments, the number of polynucleic acid molecule conjugated to a binding moiety forms a ratio. In some instances, the ratio is referred to as a DAR (drug-to-antibody ratio), in which the drug as referred to herein is the polynucleic acid molecule. In some instances, the DAR of the polynucleic acid molecule to binding moiety is about 1, 2, 3, 4, 5, 6, 7, 8, 9, 10, 11, 12, 13, 14, 15, 16, or greater. In some instances, the DAR of the polynucleic acid molecule to binding moiety is about 1 or greater. In some instances, the DAR of the polynucleic acid molecule to binding moiety is about 2 or greater. In some instances, the DAR of the polynucleic acid molecule to binding moiety is about 3 or greater. In some instances, the DAR of the polynucleic acid molecule to binding moiety is about 4 or greater. In some instances, the DAR of the polynucleic acid molecule to binding moiety is about 5 or greater. In some instances, the DAR of the polynucleic acid molecule to binding moiety is about 6 or greater. In some instances, the DAR of the polynucleic acid molecule to binding moiety is about 7 or greater. In some instances, the DAR of the polynucleic acid molecule to binding moiety is about 8 or greater. In some instances, the DAR of the polynucleic acid molecule to binding moiety is about 9 or greater. In some instances, the DAR of the polynucleic acid molecule to binding moiety is about 10 or greater. In some instances, the DAR of the polynucleic acid molecule to binding moiety is about 11 or greater. In some instances, the DAR of the polynucleic acid molecule to binding moiety is about 12 or greater.

In some instances, the DAR of the polynucleic acid molecule to binding moiety is about 1, 2, 3, 4, 5, 6, 7, 8, 9, 10, 11, 12, 13, 14, 15, or 16. In some instances, the DAR of the polynucleic acid molecule to binding moiety is about 1. In some instances, the DAR of the polynucleic acid molecule to binding moiety is about 2. In some instances, the DAR of the polynucleic acid molecule to binding moiety is about 3. In some instances, the DAR of the polynucleic acid molecule to binding moiety is about 4. In some instances, the DAR of the polynucleic acid molecule to binding moiety is about 5. In some instances, the DAR of the polynucleic acid molecule to binding moiety is about 6. In some instances, the DAR of the polynucleic acid molecule to binding moiety is about 7. In some instances, the DAR of the polynucleic acid molecule to binding moiety is about 8. In some instances, the DAR of the polynucleic acid molecule to binding moiety is about 9. In some instances, the DAR of the polynucleic acid molecule to binding moiety is about 10. In some instances, the DAR of the polynucleic acid molecule to binding moiety is about 11. In some instances, the DAR of the polynucleic acid molecule to binding moiety is about 12. In some instances, the DAR of the polynucleic acid molecule to binding moiety is about 13. In some instances, the DAR of the polynucleic acid molecule to binding moiety is about 14. In some instances, the DAR of the polynucleic acid molecule to binding moiety is about 15. In some instances, the DAR of the polynucleic acid molecule to binding moiety is about 16.

In some instances, the DAR of the polynucleic acid molecule to binding moiety is 1, 2, 3, 4, 5, 6, 7, 8, 9, 10, 11, 12, 13, 14, 15, or 16. In some instances, the DAR of the polynucleic acid molecule to binding moiety is 1. In some instances, the DAR of the polynucleic acid molecule to binding moiety is 2. In some instances, the DAR of the polynucleic acid molecule to binding moiety is 4. In some instances, the DAR of the polynucleic acid molecule to binding moiety is 5. In some instances, the DAR of the polynucleic acid molecule to binding moiety is 6. In some instances, the DAR of the polynucleic acid molecule to binding moiety is 8. In some instances, the DAR of the polynucleic acid molecule to binding moiety is 12.

In some aspects, the formulation comprises a plurality of antibody-polynucleic acid conjugates. In some instances, the plurality of antibody-polynucleic acid conjugates in the composition has different DARs. In some instances, at least two of the antibody-polynucleic acid conjugates in the composition have different DARs to each other. In some instances, the DAR is an average DAR (drug-to-antibody ratio), which is an average number of the DARs of the plurality of antibody-polynucleic acid conjugates in the composition. In some instances, the average DAR of the polynucleic acid molecule to antibody is about 1, 2, 3, 4, 5, 6, 7, 8, 9, 10, 11, 12, 13, 14, 15, 16, or greater. In some instances, the average DAR of the polynucleic acid molecule to antibody is approximately 1, 2, 3, 4, 5, 6, 7, 8, 9, 10, 11, 12, 13, 14, 15, 16, or greater. In some instances, the average DAR includes whole number as well as fractions or decimal of a DAR. In some instances, the average DAR of the polynucleic acid molecule to antibody is about 1 or greater. In some instances, the average DAR of the polynucleic acid molecule to antibody is about 2 or greater. In some instances, the average DAR of the polynucleic acid molecule to antibody is about 3 or greater. In some instances, the average DAR of the polynucleic acid molecule to antibody is about 4 or greater. In some instances, the average DAR of the polynucleic acid molecule to antibody is about 5 or greater. In some instances, the average DAR of the polynucleic acid molecule to antibody is about 6 or greater. In some instances, the average DAR of the polynucleic acid molecule to antibody is about 7 or greater. In some instances, the average DAR of the polynucleic acid molecule to antibody is about 8 or greater. In some instances, the average DAR of the polynucleic acid molecule to antibody is about 9 or greater. In some instances, the average DAR of the polynucleic acid molecule to antibody is about 10 or greater. In some instances, the average DAR of the polynucleic acid molecule to antibody is about 11 or greater. In some instances, the average DAR of the polynucleic acid molecule to antibody is about 12 or greater.

In some instances, the average DAR of the polynucleic acid molecule to antibody is about 1, 2, 3, 4, 5, 6, 7, 8, 9, 10, 11, 12, 13, 14, 15, or 16. In some instances, the average DAR of the polynucleic acid molecule to antibody is about 1. In some instances, the average DAR of the polynucleic acid molecule to antibody is about 2. In some instances, the average DAR of the polynucleic acid molecule to antibody is about 3. In some instances, the average DAR of the polynucleic acid molecule to antibody is about 4. In some instances, the average DAR of the polynucleic acid molecule to antibody is about 5. In some instances, the average DAR of the polynucleic acid molecule to antibody is about 6. In some instances, the average DAR of the polynucleic acid molecule to antibody is about 7. In some instances, the average DAR of the polynucleic acid molecule to antibody is about 7.2. In some instances, the average DAR of the polynucleic acid molecule to antibody is about 7.4. In some instances, the average DAR of the polynucleic acid molecule to antibody is about 7.6. In some instances, the average DAR of the polynucleic acid molecule to antibody is about 7.8. In some instances, the average DAR of the polynucleic acid molecule to antibody is about 8. In some instances, the average DAR of the polynucleic acid molecule to antibody is about 9. In some instances, the average DAR of the polynucleic acid molecule to antibody is about 10. In some instances, the average DAR of the polynucleic acid molecule to antibody is about 11. In some instances, the average DAR of the polynucleic acid molecule to antibody is about 12. In some instances, the average DAR of the polynucleic acid molecule to antibody is about 13. In some instances, the average DAR of the polynucleic acid molecule to antibody is about 14. In some instances, the DAR of the polynucleic acid molecule to antibody is about 15. In some instances, the average DAR of the polynucleic acid molecule to antibody is about 16.

In some instances, the average DAR of the polynucleic acid molecule to antibody is 1, 2, 3, 4, 5, 6, 7, 8, 9, 10, 11, 12, 13, 14, 15, or 16. In some instances, the average DAR of the polynucleic acid molecule to antibody is 1. In some instances, the average DAR of the polynucleic acid molecule to antibody is 2. In some instances, the average DAR of the polynucleic acid molecule to antibody is 4. In some instances, the average DAR of the polynucleic acid molecule to antibody is 6. In some instances, the average DAR of the polynucleic acid molecule to antibody is 8. In some instances, the average DAR of the polynucleic acid molecule to antibody is 12. In some instances, the average DAR of the polynucleic acid molecule to antibody is 16.

In some instances, the average DAR of the polynucleic acid molecule to antibody is in the range of 1.5-2.5, 2.5-3.5, 3.5-4.5, 4.5-5.5, 5.5-6.5, 6.5-7.5, 7.5-8.5, 8.5-9.5, 9.5-10.5, 10.5-11.5, 11.5-12.5, 12.5-13.5, 13.5-14.5, 14.5-15.5, 15.5-16.5, or 16.5-17.5. In some instances, the average DAR of the polynucleic acid molecule to antibody is in the range of 1.5-2.5. In some instances, the average DAR of the polynucleic acid molecule to antibody is in the range of 2.5-3.5. In some instances, the average DAR of the polynucleic acid molecule to antibody is in the range of 3.5-4.5. In some instances, the average DAR of the polynucleic acid molecule to antibody is in the range of 4.5-5.5. In some instances, the average DAR of the polynucleic acid molecule to antibody is in the range of 5.5-6.5. In some instances, the average DAR of the polynucleic acid molecule to antibody is in the range of 6.5-7.5. In some instances, the average DAR of the polynucleic acid molecule to antibody is in the range of 7.0-7.5. In some instances, the average DAR of the polynucleic acid molecule to antibody is in the range of 7.5-8.5. In some instances, the average DAR of the polynucleic acid molecule to antibody is in the range of 8.5-9.5. In some instances, the average DAR of the polynucleic acid molecule to antibody is in the range of 9.5-10.5. In some instances, the average DAR of the polynucleic acid molecule to antibody is in the range of 10.5-11.5. In some instances, the average DAR of the polynucleic acid molecule to antibody is in the range of 11.5-12.5. In some instances, the average DAR of the polynucleic acid molecule to antibody is in the range of 12.5-13.5. In some instances, the average DAR of the polynucleic acid molecule to antibody is in the range of 13.5-14.5. In some instances, the average DAR of the polynucleic acid molecule to antibody is in the range of 14.5-15.5. In some instances, the DAR of the polynucleic acid molecule to antibody is in the range of 15.5-16.5. In some instances, the average DAR of the polynucleic acid molecule to antibody is in the range of 16.5-17.5.

In some embodiments, an antibody or its binding fragment is further modified using conventional techniques known in the art, for example, by using amino acid deletion, insertion, substitution, addition, and/or by recombination and/or any other modification (e.g. posttranslational and chemical modifications, such as glycosylation and phosphorylation) known in the art either alone or in combination. In some instances, the modification further comprises a modification for modulating interaction with Fc receptors. In some instances, the one or more modifications include those described in, for example, International Publication No. WO97/34631, which discloses amino acid residues involved in the interaction between the Fc domain and the FcRn receptor. Methods for introducing such modifications in the nucleic acid sequence underlying the amino acid sequence of an antibody or its binding fragment is well known to the person skilled in the art.

In some instances, an antibody binding fragment further encompasses its derivatives and includes polypeptide sequences containing at least one CDR.

In some instances, the term “single-chain” as used herein means that the first and second domains of a bi-specific single chain construct are conjugated, preferably in the form of a co-linear amino acid sequence encodable by a single nucleic acid molecule.

In some instances, a bispecific single chain antibody construct relates to a construct comprising two antibody derived binding domains. In such embodiments, bi-specific single chain antibody construct is tandem bi-scFv or diabody. In some instances, a scFv contains a VH and VL domain connected by a linker peptide. In some instances, linkers are of a length and sequence sufficient to ensure that each of the first and second domains can, independently from one another, retain their differential binding specificities.

In some embodiments, binding to or interacting with as used herein defines a binding/interaction of at least two antigen-interaction-sites with each other. In some instances, antigen-interaction-site defines a motif of a polypeptide that shows the capacity of specific interaction with a specific antigen or a specific group of antigens. In some cases, the binding/interaction is also understood to define a specific recognition. In such cases, specific recognition refers to the antibody or its binding fragment is capable of specifically interacting with and/or binding to at least two amino acids of each of a target molecule. For example, specific recognition relates to the specificity of the antibody molecule, or to its ability to discriminate between the specific regions of a target molecule. In additional instances, the specific interaction of the antigen-interaction-site with its specific antigen results in an initiation of a signal, e.g. due to the induction of a change of the conformation of the antigen, an oligomerization of the antigen, etc. In further embodiments, the binding is exemplified by the specificity of a “key-lock-principle”. Thus in some instances, specific motifs in the amino acid sequence of the antigen-interaction-site and the antigen bind to each other as a result of their primary, secondary or tertiary structure as well as the result of secondary modifications of said structure. In such cases, the specific interaction of the antigen-interaction-site with its specific antigen results as well in a simple binding of the site to the antigen.

In some instances, specific interaction further refers to a reduced cross-reactivity of the antibody or its binding fragment or a reduced off-target effect. For example, the antibody or its binding fragment that bind to the polypeptide/protein of interest but do not or do not essentially bind to any of the other polypeptides are considered as specific for the polypeptide/protein of interest. Examples for the specific interaction of an antigen-interaction-site with a specific antigen comprise the specificity of a ligand for its receptor, for example, the interaction of an antigenic determinant (epitope) with the antigenic binding site of an antibody.

### Additional Binding Moieties

In some embodiments, the binding moiety is a plasma protein. In some instances, the plasma protein comprises albumin or transferrin. In some instances, the binding moiety is albumin. In some instances, the binding moiety is transferrin. In some instances, transferrin is conjugated by one or more of a conjugation chemistry described herein to a polynucleic acid molecule. In some instances, transferrin is conjugated by native ligation chemistry to a polynucleic acid molecule. In some instances, transferrin is conjugated by lysine conjugation to a polynucleic acid molecule.

In some instances, the binding moiety is a steroid. Exemplary steroids include cholesterol, phospholipids, di- and triacylglycerols, fatty acids, hydrocarbons that are saturated, unsaturated, comprise substitutions, or combinations thereof. In some instances, the steroid is cholesterol. In some instances, the binding moiety is cholesterol. In some instances, cholesterol is conjugated by one or more of a conjugation chemistry described herein to a polynucleic acid molecule. In some instances, cholesterol is conjugated by native ligation chemistry to a polynucleic acid molecule. In some instances, cholesterol is conjugated by lysine conjugation to a polynucleic acid molecule.

In some instances, the binding moiety is a polymer, including but not limited to poly nucleic acid molecule aptamers that bind to specific surface markers on cells. In this instance the binding moiety is a polynucleic acid that does not hybridize to a target gene or mRNA, but instead is capable of selectively binding to a cell surface marker similarly to an antibody binding to its specific epitope of a cell surface marker.

In some cases, the binding moiety is a peptide. In some cases, the peptide comprises between about 1 and about 3 kDa. In some cases, the peptide comprises between about 1.2 and about 2.8 kDa, about 1.5 and about 2.5 kDa, or about 1.5 and about 2 kDa. In some instances, the peptide is a bicyclic peptide. In some cases, the bicyclic peptide is a constrained bicyclic peptide. In some instances, the binding moiety is a bicyclic peptide (e.g., bicycles from Bicycle Therapeutics).

In additional cases, the binding moiety is a small molecule. In some instances, the small molecule is an antibody-recruiting small molecule. In some cases, the antibody-recruiting small molecule comprises a target-binding terminus and an antibody-binding terminus, in which the target-binding terminus is capable of recognizing and interacting with a cell surface receptor. For example, in some instances, the target-binding terminus comprising a glutamate urea compound enables interaction with PSMA, thereby, enhances an antibody interaction with a cell (e.g., a cancerous cell) that expresses PSMA. In some instances, a binding moiety is a small molecule described in Zhang et al., “A remote arene-binding site on prostate specific membrane antigen revealed by antibody-recruiting small molecules,” J Am Chem Soc. 132(36): 12711-12716 (2010); or McEnaney, et al., “Antibody-recruiting molecules: an emerging paradigm for engaging immune function in treating human disease,” ACS Chem Biol. 7(7): 1139-1151 (2012).

### Production of Antibodies or Antigen Binding Fragments Thereof

In some embodiments, polypeptides described herein (e.g., antibodies and antigen binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or antigen binding fragment thereof is generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region, e.g., humanized antibodies.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad Sci. USA 77:357; O'Hare et al., 1981, Proc. Natl. Acad Sci. USA 78:1527); gpt, which confers resistance to mycophenolic acid (Mulligan & Berg, 1981, Proc. Natl. Acad Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY; Colberre-Garapin et al., 1981, J. Mol. Biol. 150:1).

In some instances, the expression levels of an antibody are increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Lipophilic Moiety

In some embodiments, the polynucleotide conjugates described herein comprises a lipophilic moiety. In some embodiments, the lipophilic moiety is a linear or branched alky group. In some embodiments, the linear alkyl group includes, but not limited to, saturated or unsaturated C₄ to C₃₀ alkyl chains. In some embodiments, the linear alkyl group comprises unsubstituted or substituted C4, C6, C8, C10, C12, C14, C16, C18, C20, C22, C24, C26, C28 and C30 hydrocarbon chains. In some embodiments, the linear alkyl group comprises unsubstituted or substituted C14-C22 hydrocarbon chains. In some embodiments, the linear alkyl group comprises unsubstituted or substituted C14, C16, C18, C20, C22 hydrocarbon chains.

In some embodiments, the lipophilic moiety is an aliphatic, cyclic such as alicyclic, or polycyclic such as polyalicyclic compound, such as a steroid (e.g., sterol) or a linear or branched aliphatic hydrocarbon. The lipophilic moiety may generally comprise a hydrocarbon chain, which may be cyclic or acyclic. The hydrocarbon chain may comprise various substituents and/or one or more heteroatoms, such as an oxygen or nitrogen atom. Such lipophilic aliphatic moieties include, without limitation, saturated or unsaturated C₄-C₃₀ hydrocarbon (e.g., C₆-C₂₂ hydrocarbon), saturated or unsaturated fatty acids, waxes (e.g., monohydric alcohol esters of fatty acids and fatty diamides), terpenes (e.g., C₁₀ terpenes, C₁₅ sesquiterpenes, C₂₀ diterpenes, C₃₀ triterpenes, and C₄₀ tetraterpenes), and other polyalicyclic hydrocarbons. For instance, the lipophilic moiety may contain a C₄-C₃₀ hydrocarbon chain (e.g., C₄-C₃₀ alkyl or alkenyl). In some embodiment the lipophilic moiety contains a saturated or unsaturated C₆-C₁₈ hydrocarbon chain (e.g., a linear C₆-C₂₂ alkyl or alkenyl). In one embodiment, the lipophilic moiety contains a saturated or unsaturated C16 hydrocarbon chain (e.g., a linear C16 alkyl or alkenyl). In one embodiment, the lipophilic moiety contains a saturated or unsaturated C₂₂ hydrocarbon chain (e.g., a linear C₂₂ alkyl or alkenyl). In one embodiment, the lipophilic moiety is attached to the 2′-OH of a nucleotide. In one embodiment, the lipophilic moiety is attached to the 3′-OH of a nucleotide. In one embodiment, the lipophilic moiety is a lipid having an unsaturated C16 hydrocarbon chain (C16). In one embodiment, the lipophilic moiety is a lipid having an unsaturated C₂₂ hydrocarbon chain (C22).

In some embodiments, the lipophilic moiety is covalently conjugated to a polynucleotide molecule. In some embodiments, the lipophilic moiety is conjugated to a single-stranded polynucleotide molecule. In some embodiments, the lipophilic moiety is conjugated to a double-stranded polynucleotide molecule. In some embodiments, the lipophilic moiety is conjugated to an siRNA, ASO, PMO, or the like. In some embodiments, the lipophilic moiety is conjugated to an siRNA comprising a guide strand and a passenger strand. In some embodiments, the lipophilic moiety is conjugated to a guide strand of the siRNA. In some embodiments, the lipophilic moiety is conjugated to the 5′ end of the guide strand. In some embodiments, the lipophilic moiety is conjugated to the 3′ end of the guide strand. In some embodiments, the lipophilic moiety is conjugated to a passenger strand of the siRNA. In some embodiments, the lipophilic moiety is conjugated to the 5′ end of the passenger strand. In some embodiments, the lipophilic moiety is conjugated to the 3′ end of the passenger strand.

In some embodiments, the lipophilic moiety is conjugated to an internal position of the guide strand. In some embodiments, the lipophilic moiety is conjugated to a nucleotide of the guide strand at a position 1, 2, 3, 4, 5, 6, 7, 8, 9, 10, 11, 12, 13, 14, 15, 16, 17, 18, 19, 20 or 21 from the 5′ end. In some embodiments, the lipophilic moiety is conjugated to a nucleotide within the seed region of the guide strand (e.g., at positions 2-8 from the 5′ end). In some embodiments, the lipophilic moiety is conjugated to a nucleotide of the guide strand outside the seed region. In some embodiments, the lipophilic moiety is conjugated to a nucleotide of the guide strand at one of positions 16-21 from the 5′ end. In some embodiments, the lipophilic moiety is conjugated to a nucleotide of the guide strand at one of positions 7-10 from the 5′ end. In some embodiments, the lipophilic moiety is conjugated to a nucleotide of the guide strand at position 2, 3, 6, 9, 12, 17, or 19 from the 5′ end. In some embodiments, the lipophilic moiety is conjugated to a nucleotide of the guide strand at position 2, 3 or 6 of the guide strand.

In some embodiments, the lipophilic moiety is conjugated to an internal position of the passenger strand. As used herein the position of the nucleotide is counted from the 5′ end. Thus, for example, position 2 nucleotide of the passenger strand is a second nucleotide from the 5′ end of the passenger strand. In some embodiments, the lipophilic moiety is conjugated to a nucleotide at position 1, 2, 3, 4, 5, 6, 7, 8, 9, 10, 11, 12, 13, 14, 15, 16, 17, 18, or 19 of the passenger strand. In some embodiments, the lipophilic moiety is conjugated to a nucleotide at position 2, 3, 6, 9, 12, 17, or 19 of the passenger strand. In some embodiments, the lipophilic moiety is conjugated to a nucleotide at position selected from 2-8 of the passenger strand. In some embodiments, the lipophilic moiety is conjugated to a nucleotide at position 2, 3 or 6 of the passenger strand.

In some embodiments, the polynucleotide conjugate comprises one or more lipophilic moieties. In some embodiments, the one or more lipophilic moieties are same type of lipophilic moiety. For example, both lipophilic moieties are C16 saturated hydrocarbon chains. In some embodiments, the one or more lipophilic moieties are different types of lipophilic moiety. For example, one lipophilic moiety is a C16 saturated hydrocarbon chain, and another lipophilic moiety is C22 unsaturated hydrocarbon chain. In some embodiments, the polynucleotide conjugate comprises one or more lipophilic moieties that are conjugated to the polynucleotide conjugate via a single linker. In some embodiments, the polynucleotide conjugate comprises one or more lipophilic moieties that are conjugated to the polynucleotide conjugate via multiple linkers.

In some embodiments, the one or more lipophilic moieties are conjugated to one or more nucleotides at terminal and/or internal positions in the guide strand of siRNA. In some embodiments, the one or more lipophilic moieties are conjugated to one or more nucleotides at terminal and/or internal positions in the passenger strand of siRNA. In some embodiments, the one or more lipophilic moieties are conjugated to one or more nucleotides of the guide strand at positions selected from 1, 2, 3, 4, 5, 6, 7, 8, 9, 10, 11, 12, 13, 14, 15, 16, or 17 from the 5′ end. In some embodiments, the one or more lipophilic moieties are conjugated to one or more nucleotides of the passenger strand at positions selected from 1, 2, 3, 4, 5, 6, 7, 8, 9, 10, 11, 12, 13, 14, 15, 16, 17, 18 or 19 from the 5′ end. In some embodiments, the one or more lipophilic moieties are conjugated to one or more nucleotides of the guide strand at positions selected from 2, 3, 6, 9, 12, 17, or 19 from the 5′ end. In some embodiments, the one or more lipophilic moieties are conjugated to one or more nucleotides of the passenger strand at positions selected from 2, 3, 6, 9, 12, 17, or 19 from the 5′ end. In some embodiments, the one or more lipophilic moieties are conjugated to one or more nucleotides of the guide strand at positions selected from 3 or 6 from the 5′ end. In some embodiments, the one or more lipophilic moieties are conjugated to one or more nucleotides of the passenger strand at positions selected from 2, 3 or 6 from the 5′ end. In some embodiments, the one or more lipophilic moieties are conjugated to one or more nucleotides at terminal and/or internal positions in a single-stranded polynucleotide molecule, such as ASO or PMO.

In some embodiments, the lipophilic moiety is conjugated to a sugar ring of the nucleotide. In some embodiments, the lipophilic moiety is conjugated to a sugar moiety of a nucleotide of the polynucleotide molecule at the 2′-carbon or a modification made thereof. In some embodiments, the lipophilic moiety is conjugated to a sugar moiety of a nucleotide of the polynucleotide molecule at the 3′-carbon or a modification made thereof.

In some embodiments, the lipophilic moiety is conjugated to at least one 2′ modified nucleotide. In some embodiments, the at least one 2′ modified nucleotide comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In some embodiments, the at least one 2′ modified nucleotide comprises locked nucleic acid (LNA) or ethylene nucleic acid (ENA). In some embodiments, one or more lipophilic moieties are conjugated to at least one 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide. In some embodiments, the lipophilic moiety is covalently to 2′-MOE modified nucleotide.

In some embodiments, the lipophilic moiety is conjugated to a linker. In some embodiments, the lipophilic moiety is conjugated to a linker coupling the binding moiety and the polynucleotide molecule. In some embodiments, the lipophilic moiety is conjugated to a cleavable linker or a non-cleavable linker. In some embodiments, the lipophilic moiety is conjugated via a bond, C₁-C₃ M alkyl group, a homobifunctional linker or a heterobifunctional linker, optionally conjugated to C₁-C₆ alkyl group. In some embodiments, the lipophilic moiety is conjugated via homobifunctional cross linkers, heterobifunctional cross linkers, and the like. In some instances, the liker is a traceless linker (or a zero-length linker). In some instances, the linker is a non-polymeric linker. In some cases, the linker is a non-peptide linker or a linker that does not contain an amino acid residue.

In some embodiments, the lipophilic moiety is conjugated via a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[β-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the lipophilic moiety is conjugated via a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-a-methyl-a-(2-pyridyldithio)toluene (sMPT), sulfosuccinimidyl-6-[a-methyl-a-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N—(γ-maleimidobutyryloxy)succinimide ester (GMBs), N—(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)-1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (pNPDP), ρ-nitrophenyl-2-diazo-3,3,3-trifluoropropionate (PNP-DTP), sulfhydryl-reactive and photoreactive cross-linkers such asl-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some instances, the lipophilic moiety is conjugated via a reactive functional group. In some cases, the reactive functional group comprises a nucleophilic group that is reactive to an electrophilic group present on a binding moiety. Exemplary electrophilic groups include carbonyl groups-such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the lipophilic moiety is conjugated via a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of thiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide. In some embodiments, the linker comprises a bismaleimide (Bismal). In some embodiments, one or more lipophilic moieties are conjugated to any one or more linkers selected from a bismal linker, a SMCC linker, or an MBS linker.

In some embodiments, the lipophilic moiety is conjugated to a binding moiety. In some embodiments, the lipophilic moiety is conjugated to an antibody or antigen binding fragment thereof. In some embodiments, the lipophilic moiety is conjugated to an antibody to form a lipid antibody oligonucleotide conjugate (referred as “lipid AOC”). In certain embodiments, the lipophilic moiety is conjugated to an antigen binding fragment to form a lipid Fab oligonucleotide conjugate (referred as “lipid FabOC”). As used herein, “FabOC” means that a conjugate comprising an antigen binding fragment (e.g., Fab or Fab′ or any antigen binding fragment described herein) conjugated to an oligonucleotide.

In some embodiments, the lipophilic moiety is conjugated to a full length antibody. In some embodiments, the lipophilic moiety is conjugated to an antigen binding fragment. In some embodiments, the antibody or antigen binding fragment thereof comprises a humanized antibody or antigen binding fragment thereof, chimeric antibody or antigen binding fragment thereof, monoclonal antibody or antigen binding fragment thereof, monovalent Fab′, divalent Fab2, single-chain variable fragment (scFv), diabody, minibody, nanobody, single-domain antibody (sdAb), or camelid antibody or antigen binding fragment thereof. In some embodiments, the lipophilic moiety is conjugated to a monoclonal antibody. In some embodiments, the lipophilic moiety is conjugated to a Fab or Fab′.

In some embodiments, the lipophilic moiety is further modified with a triazole (tz), an oleyl, a carboxylic acid, or a combination thereof. In some embodiments, the lipophilic moiety is further modified with a triazole (tz). Exemplary triazoles include, but not limited to tzC10, tzC12, tzC14, tzC16, tzC18, tzC20, tzC22, tzC24, tzC26, tzC28, tzC30 or more.

In some embodiments, the lipophilic moiety is further modified with a triazole (tz) by click chemistry, such as copper catalyzed click chemistry, to provide high yield, specificity, and efficiency of conjugation. Examples of click chemistry include, but not limited to, Copper(I)-catalyzed azide-alkyne cycloaddition (CuAAC), Strain-promoted azide-alkyne cycloaddition (SPAAC), Strain-promoted alkyne-nitrone cycloaddition (SPANC), and the like.

In some embodiment, the lipophilic moiety is modified with a triazole (tz) prior to the antibody-oligonucleotide conjugation process. In some embodiments, the lipophilic moiety is modified with a triazole (tz) before the lipophilic moiety is conjugated to the polynucleotide. In some embodiments, the lipophilic moiety is modified with a triazole (tz) before the lipophilic moiety is conjugated to the linker. In some embodiments, the lipophilic moiety is modified with a triazole (tz) before the lipophilic moiety is conjugated to the binding moiety.

In some embodiments, the lipophilic moiety further modified with an oleyl or carboxylic acid. In some embodiments, one or more carbon atom in C₄-C₃₀ alkyl chain is modified with one or more oleic acid or carboxylic acid. In some embodiments, one or more carbon atom in C4, C6, C8, C10, C12, C14, C16, C18, C20, C22, C24, C26, C28, or C30 alkyl chain is modified with one or more oleyl or carboxylic acid. In some embodiment, the lipophilic moiety is modified with an oleyl or carboxylic acid prior to the antibody-oligonucleotide conjugation process. In some embodiments, the lipophilic moiety is modified with an oleyl or carboxylic acid before the lipophilic moiety is conjugated to the polynucleotide. In some embodiments, the lipophilic moiety is modified with an oleyl or carboxylic acid before the lipophilic moiety is conjugated to the linker. In some embodiments, the lipophilic moiety is modified with an oleyl or carboxylic acid before the lipophilic moiety is conjugated to the binding moiety.

In some embodiments, the lipophilic moiety further modified with a triazole (tz), an oleic acid, a carboxylic acid, or a combination thereof. In some embodiments, the lipophilic moiety further modified with a triazole (tz) and an oleic acid. In some embodiments, the lipophilic moiety further modified with a triazole (tz) and a carboxylic acid. In some embodiments, the lipophilic moiety further modified with an oleic acid and a carboxylic acid. In some embodiments, the lipophilic moiety further modified with a triazole (tz), an oleic acid, and a carboxylic acid. As used herein, “oleyl” and “oleic acid” means an unsaturated C18 lipid. The terms “oleyl” and “oleic acid” are used interchangeably.

In some embodiments, the length of the lipophilic moiety can be adjusted for optimal biological activity. For example, when DAR is 1, 2, 3, 4, or 5, the lipophilic moiety has a length of 16 or more. In some embodiments, the lipophilic moiety comprises C16-C30 alkyl groups. In some embodiments, the lipophilic moiety is unsubstituted or substituted C16, C17, C18, C19, C20, C21, C22, C23, C24, C25, C26, C27, C29, or C30 lipid. In some instances, when DAR is 3, 4, 5 or more, the lipophilic moiety has a length of C16 or shorter. In some embodiments, the lipophilic moiety comprises C8-C16 alkyl groups. In some embodiments, the lipophilic moiety is unsubstituted or substituted C8, C9, C10, C11, C12, C13, C14, C15 or C16 lipid.

### Linkers for Conjugating Lipophilic Moiety

In some embodiments, a linker described herein is a linker that connect the lipophilic moiety to the polynucleic acid molecule and/or the binding moiety. In some embodiments, the lipophilic moiety is conjugated to the polynucleic acid molecule via a linker. In some embodiments, the lipophilic moiety is conjugated to the binding moiety via a linker. In some embodiments, the linker is a linear linker that is capable of conjugating one lipophilic moiety to the polynucleic acid and/or the binding moiety. In some embodiments, the linker is a branched linker that is capable of conjugating a plurality of lipophilic moieties to the polynucleic acid and/or the binding moiety. In some embodiments, the linker is a branched linker that is capable of conjugating two, three, four, five, or more lipophilic moieties to the polynucleic acid and/or the binding moiety.

In some embodiments, the linker for conjugating the lipophilic moiety described herein is a compound of Formula (A) or Formula (B).

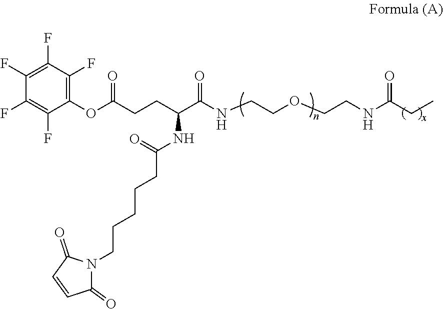

wherein n is 1-20; and x is 1-30. In some embodiments, n is 1-10; and x is 1-20. In some embodiments, n is 1, 2, 3, 4, 5, 6, 7, 8, 9, or 10. In some embodiments, n is 2, 4, 6, 8, or 10. In some embodiments, x is 1, 2, 3, 4, 5, 6, 7, 8, 9, 10, 11, 12, 13, 14, 15, 16, 17, 18, 19, or 20. In some embodiments, x is 2, 4, 6, 8, 10, 12, 14, 16, 18, or 20.

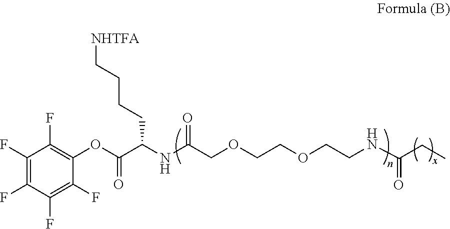

wherein n is 1-10; and x is 1-30. In some embodiments, n is 1-5; and x is 1-20. In some embodiments, n is 1, 2, 3, 4, 5, 6, 7, 8, 9, or 10. In some embodiments, n is 2, 4, 6, 8, or 10. In some embodiments, n is 1, 2, 3, 4, or 5. In some embodiments, x is 1, 2, 3, 4, 5, 6, 7, 8, 9, 10, 11, 12, 13, 14, 15, 16, 17, 18, 19, or 20. In some embodiments, x is 2, 4, 6, 8, 10, 12, 14, 16, 18, or 20.

Examples of the linkers for conjugating the lipophilic moiety are provided in Table 1 and Table 2.

TABLE 1

| Compound No. | Structure |
|---|---|
| 12a | 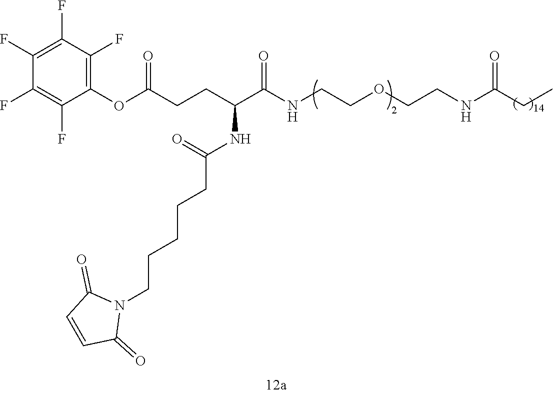 |
| 12b | 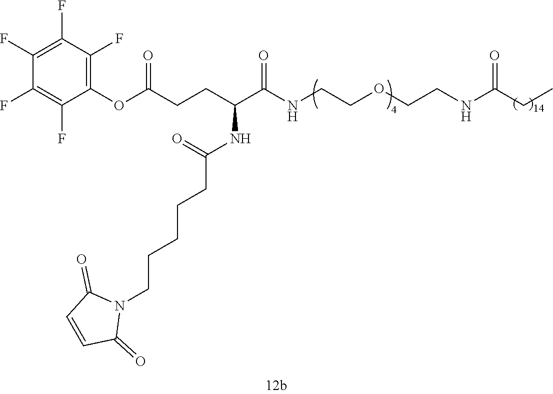 |
| 12c | 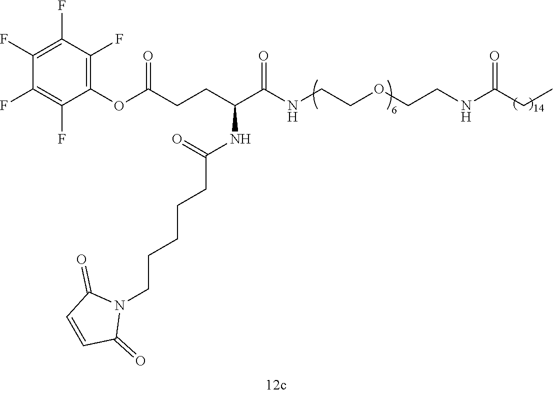 |
| 12d | 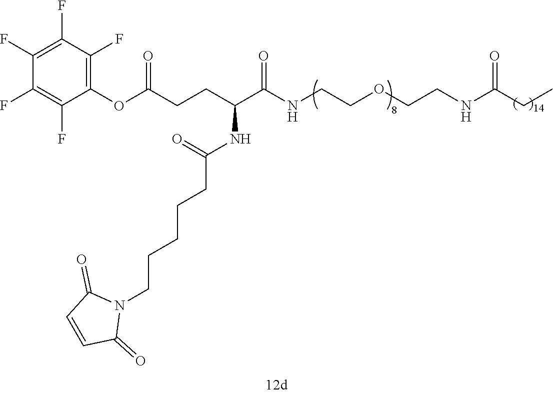 |
| 12e | 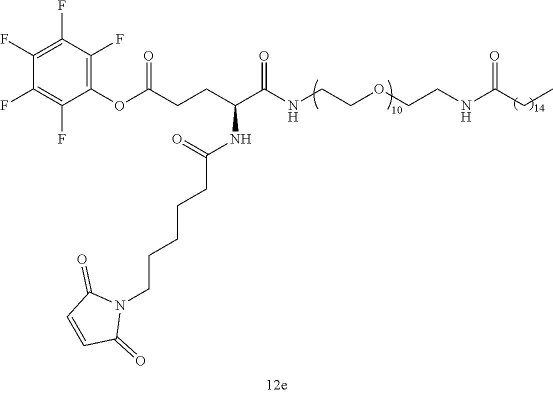 |
| 12f | 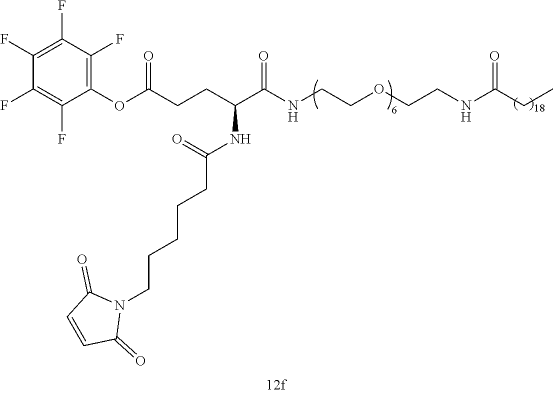 |
| 12g | 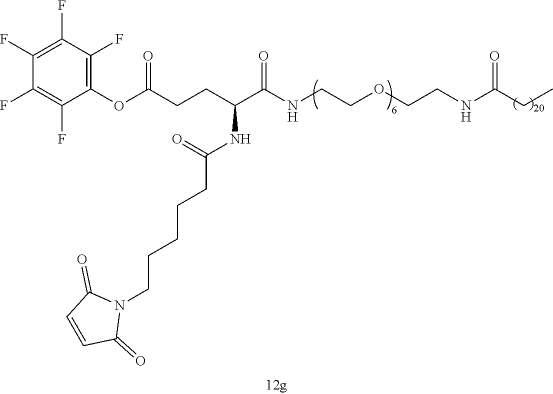 |
| 12h | 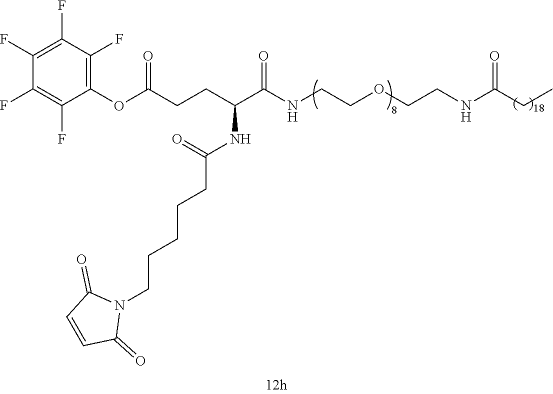 |
| 12i | 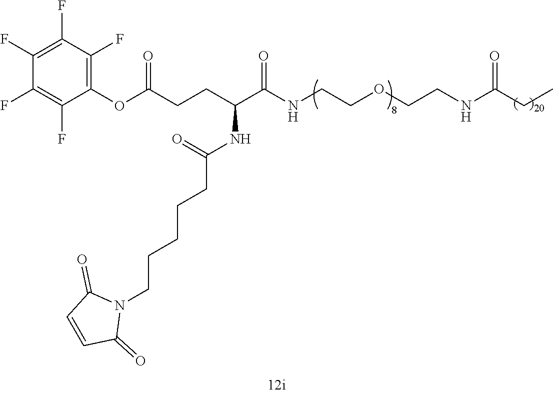 |
| 12j | 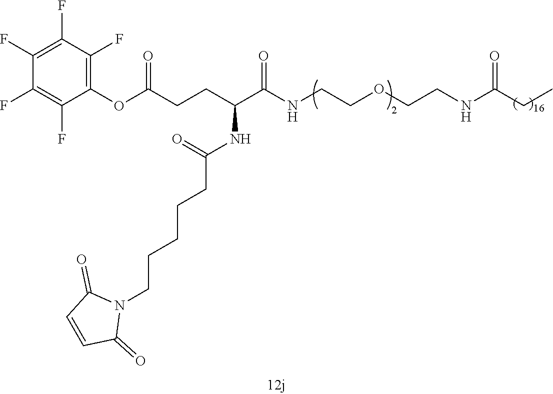 |

TABLE 2

| Compound No. | Structure |
|---|---|
| 15a | 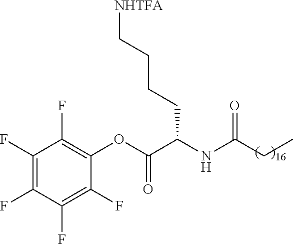 |
| | 15a |
| 15b | 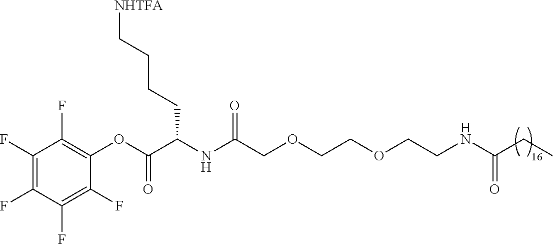 |
| | 15b |
| 15c | 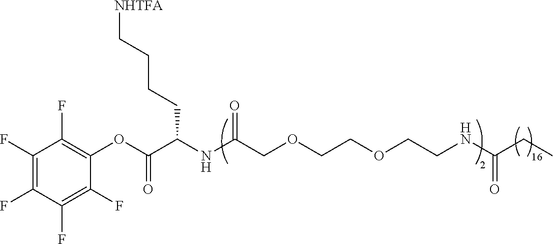 |
| | 15c |
| 15d | 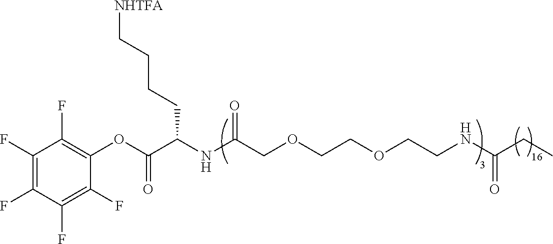 |
| | 15d |
| 15e | 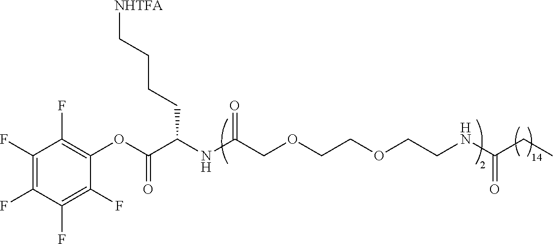 |
| | 15e |
| 15f | 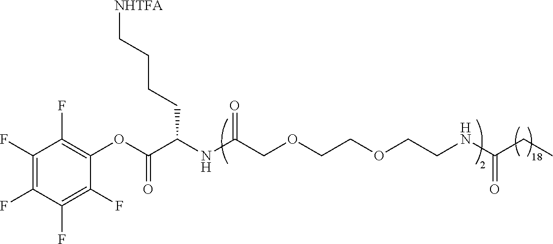 |
| | 15f |
| 15g | 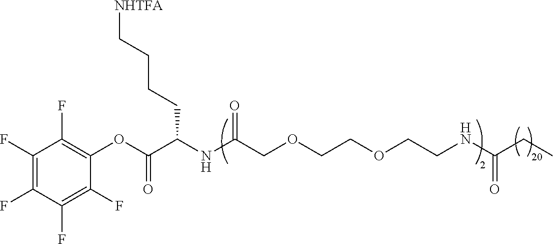 |
| | 15g |

### Linkers for Antibody Oligonucleotide Conjugate (AOC)

In some embodiments, a linker described herein is a linker that connect the binding moiety to the polynucleic acid molecule. In some embodiments, a linker described herein is a cleavable linker or a non-cleavable linker. In some instances, the linker is a cleavable linker. In some instances, the linker is an acid cleavable linker. In some instances, the linker is a non-cleavable linker. In some instances, the linker includes a C₁-C₃₀ alkyl group (e.g., a C₃₀, C₂₈, C₂₆, C₂₄, C₂₂, C₂₀, C₁₈, C16, C₁₄, C₁₂, C₁₀, C₈, C₆, C₅, C₄, C₃, C₂, or C₁ alkyl group). In some instances, the linker includes homobifunctional cross linkers, heterobifunctional cross linkers, and the like. In some instances, the liker is a traceless linker (or a zero-length linker). In some instances, the linker is a non-polymeric linker. In some cases, the linker is a non-peptide linker or a linker that does not contain an amino acid residue.

In some instances, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[P-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-a-methyl-a-(2-pyridyldithio)toluene (sMPT), sulfosuccinimidyl-6-[a-methyl-a-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N—(γ-maleimidobutyryloxy)succinimide ester (GMBs), N—(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C₂H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)-1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (pNPDP), ρ-nitrophenyl-2-diazo-3,3,3-trifluoropropionate (PNP-DTP), sulfhydryl-reactive and photoreactive cross-linkers such asl-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as p-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as ρ-azidophenyl glyoxal (APG).

In some instances, the linker comprises a reactive functional group. In some cases, the reactive functional group comprises a nucleophilic group that is reactive to an electrophilic group present on a binding moiety. Exemplary electrophilic groups include carbonyl groups-such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some instances, the maleimide group is also referred to as a maleimide spacer. In some instances, the maleimide group further encompasses a caproic acid, forming maleimidocaproyl (mc). In some cases, the linker comprises maleimidocaproyl (mc). In some cases, the linker is maleimidocaproyl (mc). In other instances, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some instances, the self-stabilizing maleimide utilizes diaminopropionic acid (DPR) to incorporate a basic amino group adjacent to the maleimide to provide intramolecular catalysis of thiosuccinimide ring hydrolysis, thereby eliminating maleimide from undergoing an elimination reaction through a retro-Michael reaction. In some instances, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some instances, the linker comprises a self-stabilizing maleimide. In some instances, the linker is a self-stabilizing maleimide. In some embodiments, the linker comprises a bismaleimide (Bismal).

In some embodiments, the linker comprises a peptide moiety. In some instances, the peptide moiety comprises at least 2, 3, 4, 5, 6, 7, 8, or more amino acid residues. In some instances, the peptide moiety is a cleavable peptide moiety (e.g., either enzymatically or chemically). In some instances, the peptide moiety is a non-cleavable peptide moiety. In some instances, the peptide moiety comprises Val-Cit (valine-citrulline), Gly-Gly-Phe-Gly (SEQ ID NO: 1), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 2), or Gly-Phe-Leu-Gly (SEQ ID NO: 3). In some instances, the linker comprises a peptide moiety such as: Val-Cit (valine-citrulline), Gly-Gly-Phe-Gly (SEQ ID NO: 1), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 2), or Gly-Phe-Leu-Gly (SEQ ID NO: 3). In some cases, the linker comprises Val-Cit. In some cases, the linker is Val-Cit.

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker is used to increase the molar ratio of polynucleotide to the binding moiety. In some instances, the dendritic type linker comprises PAMAM dendrimers.

In some embodiments, the linker is a traceless linker or a linker in which after cleavage does not leave behind a linker moiety (e.g., an atom or a linker group) to a binding moiety, a polynucleotide, a polymer, or an endosomolytic moiety. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

In some instances, the linker comprises a functional group that exerts steric hinderance at the site of bonding between the linker and a conjugating moiety. In some instances, the steric hinderance is a steric hindrance around a disulfide bond. Exemplary linkers that exhibit steric hinderance comprises a heterobifunctional linker, such as a heterobifunctional linker described above. In some cases, a linker that exhibits steric hinderance comprises SMCC and SPDB.

In some instances, the linker is an acid cleavable linker. In some instances, the acid cleavable linker comprises a hydrazone linkage, which is susceptible to hydrolytic cleavage. In some cases, the acid cleavable linker comprises a thiomaleamic acid linker. In some cases, the acid cleavable linker is a thiomaleamic acid linker as described in Castaneda, et al, “Acid-cleavable thiomaleamic acid linker for homogeneous antibody-drug conjugation,” Chem. Commun. 49: 8187-8189 (2013).

In some instances, the linker is a linker described in U.S. Pat. Nos. 6,884,869; 7,498,298; 8,288,352; 8,609,105; or 8,697,688; U.S. Patent Publication Nos. 2014/0127239; 2013/028919; 2014/286970; 2013/0309256; 2015/037360; or 2014/0294851; or PCT Publication Nos. WO2015057699; WO2014080251; WO2014197854; WO2014145090; or WO2014177042.

In some embodiments, one component of the conjugate is conjugated to another component via a bond or a linker. In some instances, the linker is a C₁-C₃₀ alkyl group. In some cases, the linker is a C₁-C₃₀ alkyl group, such as for example, a C₃₀, C₂₈, C₂₆, C₂₄, C₂₂, C₂₀, Cis, C16, C₁₄, C₁₂, C₁₀, C₈, C₆, C₅, C₄, C₃, C₂, or C₁ alkyl group. In some cases, the C₁-C₃₀ alkyl group is an unsubstituted C₁-C₃₀ alkyl group. In some embodiments, the linker is C16 linker. In some embodiments, the linker is C₂₂ linker. As used in the context of a linker, and in particular in the context of the linker, alkyl means a saturated straight or branched hydrocarbon radical containing up to six carbon atoms. In some instances, the linker is a non-polymeric linker. In some instances, the linker includes a homobifunctional linker or a heterobifunctional linker described supra. In some cases, the linker includes a heterobifunctional linker. In some cases, the linker includes sMCC. In other instances, the linker includes a heterobifunctional linker optionally conjugated to a C₁-C₆ alkyl group. In other instances, the linker includes sMCC optionally conjugated to a C₁-C₆ alkyl group. In additional instances, the linker does not include a homobifunctional linker or a heterobifunctional linker described supra.

### Methods of Use

In some embodiments, a composition or a pharmaceutical formulation described herein comprising a binding moiety conjugated to a polynucleic acid molecule and a polymer is used for the treatment of a disease or disorder. In some instances, the disease or disorder is a cancer. In some embodiments, a composition or a pharmaceutical formulation described herein is used as an immunotherapy for the treatment of a disease or disorder. In some instances, the immunotherapy is an immuno-oncology therapy.

Muscle atrophy refers to a loss of muscle mass and/or to a progressive weakening and degeneration of muscles. In some cases, the loss of muscle mass and/or the progressive weakening and degeneration of muscles occurs due to a high rate of protein degradation, a low rate of protein synthesis, or a combination of both. In some cases, a high rate of muscle protein degradation is due to muscle protein catabolism (i.e., the breakdown of muscle protein in order to use amino acids as substrates for gluconeogenesis).

In one embodiment, muscle atrophy refers to a significant loss in muscle strength. By significant loss in muscle strength is meant a reduction of strength in diseased, injured, or unused muscle tissue in a subject relative to the same muscle tissue in a control subject. In an embodiment, a significant loss in muscle strength is a reduction in strength of at least 10%, at least 15%, at least 20%, at least 25%, at least 30%, at least 35%, at least 40%, at least 45%, at least 50%, or more relative to the same muscle tissue in a control subject. In another embodiment, by significant loss in muscle strength is meant a reduction of strength in unused muscle tissue relative to the muscle strength of the same muscle tissue in the same subject prior to a period of nonuse. In an embodiment, a significant loss in muscle strength is a reduction of at least 10%, at least 15%, at least 20%, at least 25%, at least 30%, at least 35%, at least 40%, at least 45%, at least 50%, or more relative to the muscle strength of the same muscle tissue in the same subject prior to a period of nonuse.

In another embodiment, muscle atrophy refers to a significant loss in muscle mass. By significant loss in muscle mass is meant a reduction of muscle volume in diseased, injured, or unused muscle tissue in a subject relative to the same muscle tissue in a control subject. In an embodiment, a significant loss of muscle volume is at least 10%, at least 15%, at least 20%, at least 25%, at least 30%, at least 35%, at least 40%, at least 45%, at least 50%, or more relative to the same muscle tissue in a control subject. In another embodiment, by significant loss in muscle mass is meant a reduction of muscle volume in unused muscle tissue relative to the muscle volume of the same muscle tissue in the same subject prior to a period of nonuse. In an embodiment, a significant loss in muscle tissue is at least 10%, at least 15%, at least 20%, at least 25%, at least 30%, at least 35%, at least 40%, at least 45%, at least 50%, or more relative to the muscle volume of the same muscle tissue in the same subject prior to a period of nonuse. Muscle volume is optionally measured by evaluating the cross-section area of a muscle such as by Magnetic Resonance Imaging (e.g., by a muscle volume/cross-section area (CSA) MRI method).

Myotonic dystrophy is a multisystemic neuromuscular disease comprising two main types: myotonic dystrophy type 1 (DM1) and myotonic dystrophy type 2 (DM2). DM1 is caused by a dominantly inherited “CTG” repeat expansion in the gene DM protein kinase (DMPK), which when transcribed into mRNA, forms hairpins that bind with high affinity to the Muscleblind-like (MBNL) family of proteins. MBNL proteins are involved in post-transcriptional splicing and polyadenylation site regulation and loss of the MBNL protein functions lead to downstream accumulation of nuclear foci and increase in mis-splicing events and subsequently to myotonia and other clinical symptoms.

In some embodiments, described herein is a method of treating muscle atrophy or myotonic dystrophy in a subject, which comprises administering to the subject a therapeutically effective amount of a polynucleic acid molecule described herein or a polynucleic acid molecule conjugate described herein. In some instances, the muscle atrophy is associated and/or induced by cachexia (e.g., cancer cachexia), denervation, myopathy, motor neuron diseases, diabetes, chronic obstructive pulmonary disease, liver disease, congestive heart failure, chronic renal failure, chronic infection, sepsis, fasting, sarcopenia, glucocorticoid-induced atrophy, disuse, or space flight. In some cases, myotonic dystrophy is DM1.

In some embodiments, described herein is a method of delivering a polynucleotide molecule to a tissue of a subject comprising administering the polynucleotide conjugate described herein. In some embodiments, described herein is a method of increasing the stability of a polynucleotide molecule in a tissue of a subject comprising administering the polynucleotide conjugate described herein. In some embodiments, the tissue is an extrahepatic tissue. In some embodiments, the tissue is a muscle tissue. In some embodiments, the muscle tissue is a skeletal muscle tissue or a cardiac muscle tissue. In some embodiments, the higher amount of a polynucleotide molecule is maintained within the tissue compared to a polynucleotide conjugate without a lipophilic moiety at least 60, 90, or 120 days after administration.

In some embodiments, described herein is a method of modulating mRNA expression levels in a tissue of a subject comprising administering the polynucleotide conjugate described herein. In some embodiments, described herein is a method of treating muscle atrophy or myotonic dystrophy in a subject in need thereof comprising administering the polynucleotide conjugate described herein. In some embodiments, the polynucleotide conjugate is delivered to a muscle tissue. In some embodiments, the tissue is a muscle tissue. In some embodiments, the muscle tissue is a skeletal muscle tissue or a cardiac muscle tissue. In some embodiments, the polynucleotide conjugate mediates RNA interference against a target mRNA in the muscle tissue of the subject. In some embodiments, the polynucleotide conjugate decreases mRNA expression levels of a target gene in the muscle tissue of the subject. In some embodiments, the polynucleotide conjugate decreases mRNA expression levels of the target gene by 20%, 30%, 40%, 50%, 60%, 70%, 80%, or more in the muscle tissue of the subject. In some embodiments, the polynucleotide conjugate decreases mRNA expression levels of the target gene for 2 days, 28 days, 84 days or longer.

### Cachexia

Cachexia is an acquired, accelerated loss of muscle caused by an underlying disease. In some instances, cachexia refers to a loss of body mass that cannot be reversed nutritionally, and is generally associated with an underlying disease, such as cancer, COPD, AIDS, heart failure, and the like. When cachexia is seen in a patient with end-stage cancer, it is called “cancer cachexia”. Cancer cachexia affects the majority of patients with advanced cancer and is associated with a reduction in treatment tolerance, response to therapy, quality of life and duration of survival. It some instances, cancer cachexia is defined as a multifactorial syndrome characterized by an ongoing loss of skeletal muscle mass, with or without loss of fat mass, which cannot be fully reversed by conventional nutritional support and leads to progressive functional impairment. In some cases, skeletal muscle loss appears to be the most significant event in cancer cachexia. In addition, the classification of cancer cachexia suggests that the diagnostic criteria takes into account not only that weight loss is a signal event of the cachectic process but that the initial reserve of the patient should also be considered, such as low BMI or low level of muscularity.

In some embodiments, described herein is a method of treating cachexia-associated muscle atrophy in a subject, which comprises administering to the subject a therapeutically effective amount of a polynucleic acid molecule described herein or a polynucleic acid molecule conjugate described herein. In additional embodiments, described herein is a method of treating cancer cachexia-associated muscle atrophy in a subject, which comprises administering to the subject a therapeutically effective amount of a polynucleic acid molecule described herein or a polynucleic acid molecule conjugate described herein.

### Denervation

Denervation is an injury to the peripheral motoneurons with a partial or complete interruption of the nerve fibers between an organ and the central nervous system, resulting in an interruption of nerve conduction and motoneuron firing which, in turn, prevents the contractability of skeletal muscles. This loss of nerve function is either localized or generalized due to the loss of an entire motor neuron unit. The resulting inability of skeletal muscles to contract leads to muscle atrophy. In some instances, denervation is associated with or as a result of degenerative, metabolic, or inflammatory neuropathy (e.g., Guillain-Barre syndrome, peripheral neuropathy, or exposure to environmental toxins or drugs). In additional instances, denervation is associated with a physical injury, e.g., a surgical procedure.

In some embodiments, described herein is a method of treating muscle atrophy associated with or induced by denervation in a subject, which comprises administering to the subject a therapeutically effective amount of a polynucleic acid molecule described herein. In other embodiments, described herein is a method of treating muscle atrophy associated with or induced by denervation in a subject, which comprises administering to the subject a therapeutically effective amount of a polynucleic acid molecule conjugate described herein.

### Myopathy

Myopathy is an umbrella term that describes a disease of the muscle. In some instances, myopathy includes myotonia; congenital myopathy such as nemaline myopathy, multi/minicore myopathy and myotubular (centronuclear) myopathy; mitochondrial myopathy; familial periodic paralysis; inflammatory myopathy; metabolic myopathy, for example, caused by a glycogen or lipid storage disease; dermatomyositis; polymyositis; inclusion body myositis; myositis ossificans; rhabdomyolysis; and myoglobinurias. In some instances, myopathy is caused by a muscular dystrophy syndrome, such as Duchenne, Becker, myotonic, facioscapulohumeral, Emery-Dreifuss, oculopharyngeal, scapulohumeral, limb girdle, Fukuyama, a congenital muscular dystrophy, or hereditary distal myopathy. In some instances, myopathy is caused by myotonic dystrophy (e.g., myotonic dystrophy type 1 or DM1). In some instances, myopathy is caused by DM1.

In some embodiments, described herein is a method of treating muscle atrophy associated with or induced by myopathy in a subject, which comprises administering to the subject a therapeutically effective amount of a polynucleic acid molecule described herein. In other embodiments, described herein is a method of treating muscle atrophy associated with or induced by myopathy in a subject, which comprises administering to the subject a therapeutically effective amount of a polynucleic acid molecule conjugate described herein.

### Pharmaceutical Formulation

In some embodiments, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular) administration. In other instances, the pharmaceutical composition described herein is formulated for oral administration. In still other instances, the pharmaceutical composition described herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate-release formulations, controlled-release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation includes multiparticulate formulations. In some instances, the pharmaceutical formulation includes nanoparticle formulations. In some instances, nanoparticles comprise cMAP, cyclodextrin, or lipids. In some cases, nanoparticles comprise solid lipid nanoparticles, polymeric nanoparticles, self-emulsifying nanoparticles, liposomes, microemulsions, or micellar solutions. Additional exemplary nanoparticles include, but are not limited to, paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. In some instances, a nanoparticle is a metal nanoparticle, e.g., a nanoparticle of scandium, titanium, vanadium, chromium, manganese, iron, cobalt, nickel, copper, zinc, yttrium, zirconium, niobium, molybdenum, ruthenium, rhodium, palladium, silver, cadmium, hafnium, tantalum, tungsten, rhenium, osmium, iridium, platinum, gold, gadolinium, aluminum, gallium, indium, tin, thallium, lead, bismuth, magnesium, calcium, strontium, barium, lithium, sodium, potassium, boron, silicon, phosphorus, germanium, arsenic, antimony, and combinations, alloys or oxides thereof.

In some instances, a nanoparticle includes a core or a core and a shell, as in a core-shell nanoparticle.

In some instances, a nanoparticle is further coated with molecules for attachment of functional elements (e.g., with one or more of a polynucleic acid molecule or binding moiety described herein). In some instances, a coating comprises chondroitin sulfate, dextran sulfate, carboxymethyl dextran, alginic acid, pectin, carragheenan, fucoidan, agaropectin, porphyran, karaya gum, gellan gum, xanthan gum, hyaluronic acids, glucosamine, galactosamine, chitin (or chitosan), polyglutamic acid, polyaspartic acid, lysozyme, cytochrome C, ribonuclease, trypsinogen, chymotrypsinogen, a-chymotrypsin, polylysine, polyarginine, histone, protamine, ovalbumin, dextrin, or cyclodextrin. In some instances, a nanoparticle comprises a graphene-coated nanoparticle.

In some cases, a nanoparticle has at least one dimension of less than about 500 nm, 400 nm, 300 nm, 200 nm, or 100 nm.

In some instances, the nanoparticle formulation comprises paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes or quantum dots. In some instances, a polynucleic acid molecule or a binding moiety described herein is conjugated either directly or indirectly to the nanoparticle. In some instances, at least 1, 5, 10, 15, 20, 30, 40, 50, 60, 70, 80, 90, 100, or more polynucleic acid molecules or binding moieties described herein are conjugated either directly or indirectly to a nanoparticle.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some instances, the pharmaceutical formulations further include pH-adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, dimethyl isosorbide, and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens

In some embodiments, the pharmaceutical compositions described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In some embodiments, one or more pharmaceutical compositions are administered simultaneously, sequentially, or at an interval period of time. In some embodiments, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition).

In some embodiments, two or more different pharmaceutical compositions are coadministered. In some instances, the two or more different pharmaceutical compositions are coadministered simultaneously. In some cases, the two or more different pharmaceutical compositions are coadministered sequentially without a gap of time between administrations. In other cases, the two or more different pharmaceutical compositions are coadministered sequentially with a gap of about 0.5 hour, 1 hour, 2 hour, 3 hour, 12 hours, 1 day, 2 days, or more between administrations.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, are optionally reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include a molecule as disclosed herein. Such kits optionally include an identifying description or label or instructions relating to its use in the methods described herein.

A kit typically includes labels listing contents and/or instructions for use and package inserts with instructions for use. A set of instructions will also typically be included.

In one embodiment, a label is on or associated with the container. In one embodiment, a label is on a container when letters, numbers, or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that is expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “polynucleotide,” “polynucleotide molecule,” “polynucleic acid molecule” and “oligonucleotide,” are used interchangeably to refer a chain of nucleotides.

The term “antibody” is used in the broadest sense and covers fully assembled antibodies, antibody fragments that can bind antigen (e.g., Fab, F(ab′)₂, Fv, single chain antibodies, VHH, diabodies, antibody chimeras, hybrid antibodies, bispecific antibodies, humanized antibodies, and the like), and recombinant peptides comprising the forgoing.

The terms “antigen binding fragments” and “antibody fragments” are used interchangeably to refer a portion of an intact antibody, preferably the antigen-binding or variable region of the intact antibody. Examples of antibody fragments include Fab, Fab, F(ab′)₂, and Fv fragments; VHH; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

Depending on the amino acid sequence of the constant domain of their heavy chains, immunoglobulins can be assigned to different classes. There are five major classes of human immunoglobulins: IgA, IgD, IgE, IgG, IgM, and IgY, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

In some instances, the CDRs of an antibody is determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M. P., 1999, The Immunologist, 7: 132-136 and Lefranc, M. P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

The term “chimeric” antibody refers to an antibody in which a portion of the heavy and/or light chain is derived from a particular source or species, while the remainder of the heavy and/or light chain is derived from a different source or species.

The term “humanized antibody” refers to antibodies in which the framework or the CDRs have been modified to comprise the CDR of an immunoglobulin of different specificity as compared to that of the parent immunoglobulin.

The term “alkyl,” by itself or as part of another substituent, means, unless otherwise stated, a straight (i.e., unbranched) or branched carbon chain (or carbon), or combination thereof, which may be fully saturated, mono- or poly-unsaturated and can include mono-, di-, and multivalent radicals, having the number of carbon atoms designated (i.e., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. “Cycloalkyl” is also meant to refer to bicyclic and polycyclic hydrocarbon rings such as, for example, bicyclo[2.2.1]heptane, bicyclo[2.2.2]octane, etc.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted, and each substituent may optionally be different.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but are not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, or —NR′OR″, in a number ranging from zero to (2m′+1), where m′ is the total number of carbon atoms in such radical. R, R′, R″, R′″, and R¹¹ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N3, —CH(Ph)₂, fluoro(C₁-C₄)alkoxy, and fluoro(C₁-C₄)alkyl, —NR′SO₂R″, —NR′C(O)R″, —NR′C(O)—OR″, or —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituent (including, but are not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

In other aspects of the compounds herein, each substituted or unsubstituted alkyl may be a substituted or unsubstituted C₁-C₂₀ alkyl, each substituted or unsubstituted heteroalkyl is a substituted or unsubstituted 2- to 20-membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3- to 8-membered heterocycloalkyl, each substituted or unsubstituted aryl is a substituted or unsubstituted C₆-C₁₀ aryl, and/or each substituted or unsubstituted heteroaryl is a substituted or unsubstituted 5- to 10-membered heteroaryl. In some aspects of the compounds herein, each substituted or unsubstituted alkylene is a substituted or unsubstituted C₁-C₂₀ alkylene, each substituted or unsubstituted heteroalkylene is a substituted or unsubstituted 2- to 20-membered heteroalkylene, each substituted or unsubstituted cycloalkylene is a substituted or unsubstituted C₃-C₅ cycloalkylene, each substituted or unsubstituted heterocycloalkylene is a substituted or unsubstituted 3- to 8-membered heterocycloalkylene, each substituted or unsubstituted arylene is a substituted or unsubstituted C₆-C₁₀ arylene, and/or each substituted or unsubstituted heteroarylene is a substituted or unsubstituted 5- to 10-membered heteroarylene.

In some certain aspects, each substituted or unsubstituted alkyl is a substituted or unsubstituted C₁-C₅ alkyl, each substituted or unsubstituted heteroalkyl is a substituted or unsubstituted 2- to 8-membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3- to 7-membered heterocycloalkyl, each substituted or unsubstituted aryl is a substituted or unsubstituted C₆-C₁₀ aryl, and/or each substituted or unsubstituted heteroaryl is a substituted or unsubstituted 5- to 9-membered heteroaryl. In some certain aspects, each substituted or unsubstituted alkylene is a substituted or unsubstituted C₁-C₈ alkylene, each substituted or unsubstituted heteroalkylene is a substituted or unsubstituted 2- to 8-membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3- to 7-membered heterocycloalkylene, each substituted or unsubstituted arylene is a substituted or unsubstituted C₆-C₁₀ arylene, and/or each substituted or unsubstituted heteroarylene is a substituted or unsubstituted 5- to 9 membered heteroarylene. In some aspects, the compound is a chemical species set forth in the Examples section, figures, or tables below.

The terms “disease” or “condition” refer to a state of being or health status of a patient or subject capable of being treated with the compounds or methods provided herein. The disease may be a cancer.

The terms “treating” or “treatment” refer to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters, including the results of a physical examination, neuropsychiatric exams, and/or a psychiatric evaluation. The term “treating” and conjugations thereof, may include prevention of an injury, pathology, condition, or disease. In certain aspects, treating is preventing. In certain aspects, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of a compound described herein. The administering step may consist of a single administration or may include a series of administrations. The length of the treatment period depends on a variety of factors, such as the severity of the condition, the age of the patient, the concentration of the compound, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. As indicated above, the prevention may be complete (no detectable symptoms) or partial, such that fewer symptoms are observed than would likely occur absent treatment. In certain aspects, prevent refers to slowing the progression of the disease, disorder or condition or inhibiting progression thereof to a harmful or otherwise undesired state.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

As used herein, the term “sense strand” can be interchangeably used with the term “passenger strand,” and the term “antisense strand” can be interchangeably used with the term “guide strand.”

As used herein, the term “comprising” and its derivatives are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

As used herein, the term “consisting of” and its derivatives are intended to be closed ended terms that specify the presence of stated features, elements, components, groups, integers, and/or steps, and also exclude the presence of other unstated features, elements, components, groups, integers and/or steps.

As used herein, the term “consisting essentially of” is intended to specify the presence of the stated features, elements, components, groups, integers, and/or steps as well as those that do not materially affect the basic and novel characteristic(s) of features, elements, components, groups, integers, and/or steps.

### EXAMPLES

These examples are provided for illustrative purposes only and not to limit the scope of the claims provided herein.

### Example 1: Antibody-Oligonucleotide Synthesis

### siRNA Synthesis

siRNAs were synthesized following standard solid phase synthesis methods with a MerMade12 synthesizer on standard 2′-O-methyl-base loaded 1000 Å CPG solid support. 2′-O-methyl, 2′-fluoro and 2′-O—C16 phosphonamidites were dissolved in anhydrous acetonitrile or anhydrous dichloromethane to make 0.1 M solution. 3% Trichloroacetic Acid in dichloromethane, 0.25 M 5-ethylthio tetrazole in acetonitrile, 0.02 M iodine in pyridine/THF/H2O, 0.05 M (dimethylamino-methylidene)amino)-3H-1,2,4-dithiazoline-3-thione (DDTT) in pyridine/acetonitrile, acetic anhydride/THF/2,6-lutidine (Cap A) and 16% N-methylimidazole in THF (Cap B) were used as deblocking, activation, oxidation, sulfurization and capping reagent, respectively. After synthesis, solid supports were dried and incubated in ammonium hydroxide/40% aqueous methylamine solution (AMA) for 2 hr at room temperature. The CPG was filtered out and washed with water/methanol and the combined filtrate was concentrated under vacuum.

Single-stranded nucleotides were purified by reverse-phase HPLC or anion exchange chromatography and desalted. Purified single-stranded nucleotides were duplexed to make double-stranded siRNA. siRNA passenger strands were synthesized with or without a C6-NH2 conjugation handle at the 5′ end, attached through a phosphodiester or phosphorothioate linkage. siRNA guide strands were synthesized with or without a 5′-(E)-vinyl phosphonate modified nucleotide. Both single-stranded nucleotides were identified by mass spectroscopy and their purity was assessed by HPLC analysis. Size exclusion chromatography (SEC) was used for duplex analysis.

To produce the triazole-lipid single strands, the 2′-propargyl modified single-stranded nucleotide in water was added to solution of DMSO containing 20 equivalents of the azide-modified lipid. To the solution was added a 1:1 pre-mixed solution (v/v) of copper sulfate (20 equivalents in water) and N-((1H-Benzo[d]imidazol-2-yl)methyl)-1-(pyridin-2-yl)-N-(pyridin-2-ylmethyl)methanamine (30 equivalents in DMSO), sodium ascorbate (100 equivalents in water), and sufficient volume of DMSO to bring the reaction volume to 75% DMSO. The reaction mixture was heated to 60° C. for 30 minutes before addition of 100 equivalents of an immobilized Cu scavenger (SiliCycle TAAcONa, 40 to 63 m, 60 Å). The solution was incubated at room temperature for 2 hours and the solids were removed by centrifugation and the supernatant removed, diluted to 5% DMSO (v/v) in water, and purified with spin filtration (5×) using 3K MWCO filters. The clicked lipid-single stranded nucleotide was purified with reverse-phase HPLC with Method 1 and desalted prior to analysis by LCMS.

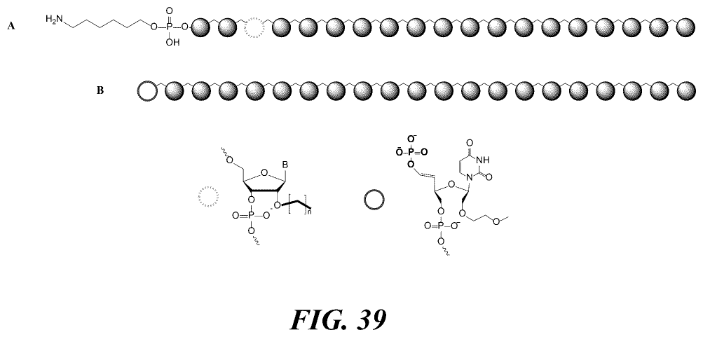

Representative structures of A. siRNA passenger strand with a 2′O-lipid conjugated nucleotide at position 3 and a 5′ C6-NH2 conjugation handle and B. siRNA guide strand with a 5′ (E) vinyl phosphonate modification are shown in FIG. 39.

### Single-Stranded Nucleotide Purification Methods

Method 1: Ion-pair reverse phase chromatography
Column: XBridge Prep C18 Sum OBD 19×250 mm Waters
Buffer A: 50 mM TEAA in Water
Buffer B: Acetonitrile
Gradient: 5%-20% B in 35 min, 20 mL/min
Gradient: 10%-40% B in 45 min, 20 mL/min (for lipid containing single sequences)

### Method 2: Anion Exchange Chromatography

Column: TSKgel SuperQ-5PW 13 μm 21.5 mm I.D×15 cm
Buffer A: 20 mM Sodium phosphate pH 7.2
Buffer B: 1 M NaBr in 20 mM Sodium phosphate pH 7.2
Gradient: 25%-35% B in 35 min, 20 mL/min

### Analytical Methods

### Single-Stranded Nucleotide Analysis

Column: ACQUITY UPLC Oligonucleotide BEH C18 Column, 130A, 1.7 um 2.1 mm×50 mm
Temp: 70° C.
Buffers: Buffer A: 2% HFIP, 0.2% Hexylamine in water, Buffer B: ACN/MeOH (1:1)
Gradient: 5-50% 20 min, 0.3 mL/min
   siRNA Analysis

### Method-1: Ion-Pair Reverse Phase Chromatography (IP-UPLC)

System: Agilent 1290 Infinity II ACQUITY UPLC Oligonucleotide BEH C18 Column, 130A, 1.7 um
Column: 2.1 mm×50 mm
Buffers: Buffer A: 1% HFIP, 0.2% TEA, 50 uM EDTA in water, Buffer B: MeOH
Gradient: 5-50% 20 min, 0.3 mL/min

### Method-2: Size Exclusion Chromatography (SEC)

Column: ACQUITY UPLC Protein BEH SEC-Analytical HPLC
Buffer: 200 mM Tris buffer, pH 7.2, 5% Acetonitrile
Flowrate: 1 mL/min, 10 min
   siRNA Sequences

The sequence of the guide/antisense strand was complementary to the gene sequence. For example, the guide strand starting at base position 1169 for the mouse mRNA transcript MSTN is UUAUUAUUUGUUCUUUGCCUU (SEQ ID NO: 4). Base, sugar, and phosphate modifications were used to optimize the potency of the duplex and reduce immunogenicity. The guide strand starting at base position 1 for the mouse mRNA transcript SSB is UUACAUUAAAGUCUGUUGUUU (SEQ ID NO: 5). The guide strand starting at base position 2628 for the mouse mRNA transcript DMPK is UAAUACCGAGGAAUGUCGGUU (SEQ ID NO: 6).

### Anti-Transferrin Receptor Antibody

Anti-mouse transferrin receptor antibody or CD71 mAb is a rat IgG2a subclass monoclonal antibody that binds mouse CD71 or mouse transferrin receptor 1 (mTfR1). The antibody was produced by BioXcell and it is commercially available (Catalog #BE0175).

### Anti-Transferrin Receptor Antibody Fragment (Fab)

Anti-mouse transferrin receptor antibody fragment (Fab) was produced by WuXi in CHO cells and purified using a protein L resin. If the Fab purity was <95%, the Fab was then purified by an anti-human CHI resin to remove excess light chain and/or polished using a size exclusion chromatography.

### IgG2a Isotype Control Antibody

Rat IgG2a isotype control antibody was purchased from BioXcell (Clone 2A3, Catalog #BE0089) and this antibody is specific to trinitrophenol and does not have any known antigens in mouse.

Antibody siRNA Conjugate Synthesis

### DAR1 and DAR2 Antibody-siRNA Conjugate Synthesis Using Bismal Linker

Step 1: Addition of Bismal Linker to siRNA Duplex

BisMal-dPEG4-TFP was purchased from Quanta Biosciences (Catalog #QBD-10631) and a stock solution was prepared at 60 mg/ml in dimethyl sulfoxide (DMSO). Duplexed siRNA in deionized water was added to 1 M Phosphate Buffer, pH 7.2 to a final concentration of 50 mM phosphate buffer and the pH adjusted to 7.6. DMSO was added to the siRNA solution to bring the final concentration DMSO in the reaction to 60% and 20 equivalents of the bismal linker. The reaction was allowed to proceed for 1 hr before dilution to 5% DMSO in cold 10 mM acetate buffer, pH 6. The diluted reaction mixture was purified by spin filtration (5×) using 3K MWCO filters. The linker-modified duplex was analyzed using LCMS and stored at −20° C. until reaction with reduced antibody.

Step 2: Antibody Interchain Disulfide Reduction with TCEP

To a solution of antibody in phosphate buffered saline (PBS, pH 7.4) at 6.96 mg/ml protein concentration was added sufficient 0.5 M ethylenediaminetetraacetic acid (EDTA) to afford a final concentration of 2 mM EDTA in the final reaction solution. To the solution containing antibody and EDTA was added 6 equivalents of tris(2-carboxyethyl)phosphine hydrochloride (TCEP) in water and incubated for 4 hours at 37° C. To the resultant reduced antibody solution was added a solution of bismal-C₆-siRNA (2 equivalents) in 10 mM sodium acetate buffer, pH 6. The reaction proceeded for 1 hour before quenching with 10 equivalents of N-ethyl Maleimide (NEM) as a 10 mg/ml stock solution in dimethyl sulfoxide (DMSO). Analysis of the reaction mixture by analytical SAX and SEC column chromatography showed antibody siRNA conjugate along with unreacted antibody and siRNA.

### Step 3: Purification of Antibody-siRNA Conjugate

The crude reaction mixture was purified by AKTA explorer FPLC using anion exchange chromatography method-1. Fractions containing DAR1 and DAR2 conjugates were separated, concentrated and buffer exchanged with pH 7.4 PBS.

### Step 4: Analysis of the Purified Conjugate

The isolated conjugates were characterized by SEC with SEC using size exclusion chromatography and SAX chromatography anion exchange chromatography method-3 or method-5 as described in Purification and Analytical Methods section. All methods are described in Purification and Analytical Methods. Isolated conjugates had greater than 90% purity.

### Example 2: DAR1 and DAR2 Antibody-siRNA Conjugate Synthesis Using SMCC Linker

Step 1: Addition of SMCC Linker to siRNA Duplex

SMCC was purchased from ThermoFisher (Catalog #22360) and a stock solution was prepared at 60 mg/ml in dimethyl sulfoxide (DMSO). To duplexed siRNA in deionized water was added 1 M Phosphate Buffer, pH 7.2 to a final concentration of 50 mM phosphate buffer and the pH adjusted to 7.6. To the duplex was added sufficient DMSO to bring the final concentration DMSO in the reaction to 60% (v/v) and 10 equivalents of the SMCC linker. The reaction was allowed to proceed for 1 hr before dilution to 5% DMSO in cold 10 mM acetate buffer, pH 6. The diluted reaction mixture was purified by spin filtration (5×) using 3K MWCO filters. The linker-modified duplex was analyzed using LCMS and stored at −20° C. until reaction with reduced antibody.

Step 2: Antibody Interchain Disulfide Reduction with TCEP

To a solution of antibody in phosphate buffered saline (PBS, pH 7.4) at 6.96 mg/ml protein concentration was added sufficient 0.5 M ethylenediaminetetraacetic acid (EDTA) to afford a final concentration of 2 mM EDTA in the final reaction solution. To the solution containing antibody and EDTA was added 6 equivalents of tris(2-carboxyethyl)phosphine hydrochloride (TCEP) in water and incubated for 4 hours at 37° C. To the resultant reduced antibody solution was added a solution of SMCC-C6-siRNA (2 equivalents) in 10 mM sodium acetate buffer, pH 6. The reaction proceeded for 1 hour before quenching with 10 equivalents of N-ethyl maleimide (NEM) as a 10 mg/ml stock solution in dimethyl sulfoxide (DMSO). Analysis of the reaction mixture by analytical SAX and SEC column chromatography showed antibody siRNA conjugate along with unreacted antibody and siRNA.

Step 3: Purification of Antibody siRNA Conjugate

The crude reaction mixture was purified by AKTA explorer FPLC using anion exchange chromatography method-1. Fractions containing DAR1 and DAR2 conjugates were separated, concentrated and buffer exchanged with pH 7.4 PBS.

### Step 4: Analysis of the Purified Conjugate

The isolated conjugates were characterized using size exclusion chromatography and SAX chromatography anion exchange chromatography method-3 or method-5 as described in the corresponding section. All methods are described in Purification and Analytical Methods section herein. Isolated conjugates had greater than 90% purity.

### DAR1 and DAR2 Monoclonal Antibody-siRNA Conjugate Synthesis Using MBS Linker

Step 1: Addition of MBS Linker to siRNA Duplex

m-Maleimidobenzoyl-N-hydroxysuccinimide ester (MBS) was purchased from ThermoFisher (Catalog #22311) and a stock solution was prepared at 60 mg/ml in dimethyl sulfoxide (DMSO). To duplexed siRNA in deionized water was added 1 M phosphate buffer, pH 7.2 to a final concentration of 50 mM phosphate buffer and the pH adjusted to 7.6. DMSO was added to the duplexed siRNA solution to bring the final concentration DMSO in the reaction to 60% (v/v) and 10 equivalents of the MBS linker at 0° C. The reaction was allowed to proceed for 1 hr before dilution to 5% DMSO in cold 10 mM acetate buffer, pH 6. The diluted reaction mixture was purified by spin filtration (5×) using 3K MWCO filters. The linker-modified duplex was analyzed using LCMS and stored at −20° C. until reaction with reduced antibody.

Step 2: Antibody Interchain Disulfide Reduction with TCEP

To a solution of antibody in phosphate buffered saline (PBS, pH 7.4) at 6.96 mg/ml protein concentration was added sufficient 0.5 M ethylenediaminetetraacetic acid (EDTA) to afford a final concentration of 2 mM EDTA in the final reaction solution. To the solution containing antibody and EDTA was added 6 equivalents of tris(2-carboxyethyl)phosphine hydrochloride (TCEP) in water and incubated for 4 hours at 37° C. To the resultant reduced antibody solution was added a solution of MBS-C₆-siRNA (2 equivalents) in 10 mM sodium acetate buffer, pH 6. The reaction proceeded for 1 hour before quenching with 10 equivalents of N-ethyl maleimide (NEM) as a 10 mg/ml stock solution in dimethyl sulfoxide (DMSO). Analysis of the reaction mixture by analytical SAX and SEC column chromatography showed antibody siRNA conjugate along with unreacted antibody and siRNA.

### Step 3: Purification of Antibody-siRNA Conjugate

The crude reaction mixture was purified by AKTA explorer FPLC using anion exchange chromatography method-1. Fractions containing DAR1 and DAR2 conjugates were separated, concentrated and buffer exchanged with pH 7.4 PBS.

### Step 4: Analysis of the Purified Conjugate

The isolated conjugates were characterized using size exclusion chromatography method-1 and SAX chromatography anion exchange chromatography method-3 or method-5 as described in Purification and Analytical Methods section herein. Isolated conjugates had greater than 90% purity.

### DAR3+ Monoclonal Antibody-siRNA Conjugate Synthesis Using SMCC Linker

Step 1: Addition of SMCC Linker to siRNA Duplex

SMCC was purchased from ThermoFisher (Catalog #22360) and a stock solution was prepared at 60 mg/ml in dimethyl sulfoxide (DMSO). To duplexed siRNA in deionized water was added 1 M phosphate buffer, pH 7.2 to a final concentration of 50 mM phosphate buffer and the pH adjusted to 7.6. To the duplex was added sufficient DMSO to bring the final concentration DMSO in the reaction to 60% (v/v) and 10 equivalents of the SMCC linker. The reaction was allowed to proceed for 1 hr before dilution to 5% DMSO in cold 10 mM acetate buffer, pH 6. The diluted reaction mixture was purified by spin filtration (5×) using 3K MWCO filters. The linker-modified duplex was analyzed using LCMS and stored at −20° C. until reaction with reduced antibody.

Step 2: Antibody Interchain Disulfide Reduction with TCEP

To a solution of antibody in phosphate buffered saline (PBS, pH 7.4) at 17.18 mg/ml protein concentration was added sufficient 0.5 M ethylenediaminetetraacetic acid (EDTA) to afford a final concentration of 2 mM EDTA in the final reaction solution. To the solution containing antibody and EDTA was added tris(2-carboxyethyl)phosphine hydrochloride (TCEP) (6 equivalents for DAR4, 10 equivalents for DAR5) in water and incubated for 4 hours at 37° C. To the resultant reduced antibody solution was added a solution of SMCC-C6-siRNA (7 equivalents for DAR4, 15 equivalents for DAR5) in 10 mM sodium acetate buffer, pH 6. The reaction proceeded for 1 hour before quenching with 10 equivalents of N-ethyl Maleimide (NEM) as a 10 mg/ml stock solution in dimethyl sulfoxide (DMSO). Analysis of the reaction mixture by analytical SAX and SEC column chromatography showed antibody siRNA conjugate along with unreacted antibody and siRNA.

Step 3: Purification of Antibody siRNA Conjugate

The crude reaction mixture was purified by spin filtration against PBS 10× using an Amicon Ultra Centrifugal Filter, 50 kDa MWCO from Millipore Sigma (Catalog #UFC9050).

### Step 4: Analysis of the Purified Conjugate

The isolated conjugates were characterized by SEC with SEC using size exclusion chromatography method-1 and SAX chromatography anion exchange chromatography method-4 as described in Purification and Analytical Methods section herein. Isolated conjugates had greater than 90% purity.

### Example 3. Fab-siRNA Conjugate Synthesis with C16 Lipid Containing Linker Fab-siRNA Conjugate Synthesis Using SMCC Linker

Step 1: Addition of SMCC Linker to siRNA Duplex

SMCC was purchased from ThermoFisher (Catalog #22360) and a stock solution was prepared at 60 mg/ml in dimethyl sulfoxide (DMSO). To duplexed siRNA in deionized water was added 1 M phosphate buffer, pH 7.2 to a final concentration of 50 mM phosphate buffer and the pH adjusted to 7.6. To the duplex was added sufficient DMSO to bring the final concentration DMSO in the reaction to 60% (v/v) and 10 equivalents of the SMCC linker. The reaction was allowed to proceed for 1 hr before dilution to 5% DMSO in cold 10 mM acetate buffer, pH 6. The diluted reaction mixture was purified by spin filtration (5×) using 3K MWCO filters. The linker-modified duplex was analyzed using LCMS and stored at −20° C. until reaction with reduced antibody.

Step 2: Antibody Interchain Disulfide Reduction with TCEP

To a solution of antibody in phosphate buffered saline (PBS, pH 7.4) at 26.18 mg/ml protein concentration was added sufficient 0.5 M ethylenediaminetetraacetic acid (EDTA) to afford a final concentration of 2 mM EDTA in the final reaction solution. To the solution containing antibody and EDTA was added 1.5 equivalents of tris(2-carboxyethyl)phosphine hydrochloride (TCEP) in water and incubated for 4 hours at 37° C. To the resultant reduced antibody solution was added a solution of SMCC-C6-siRNA (2 equivalents) in 10 mM sodium acetate buffer, pH 6. The reaction proceeded for 16 hours at room temperature before quenching with 10 equivalents of N-ethyl maleimide (NEM) as a 10 mg/ml stock solution in dimethyl sulfoxide (DMSO). Analysis of the reaction mixture by analytical SAX and SEC column chromatography showed antibody siRNA conjugate along with unreacted antibody and siRNA.

Step 3: Purification of Antibody siRNA Conjugate.

The crude reaction mixture was purified by AKTA explorer FPLC using anion exchange chromatography method-2. Fractions containing DAR1 and DAR2 conjugates were separated, concentrated and buffer exchanged with pH 7.4 PBS.

### Step 4: Analysis of the Purified Conjugate

The isolated conjugates were characterized by SEC with SEC using size exclusion chromatography method-2 and SAX chromatography anion exchange chromatography method-3 as described in the corresponding section. All methods are described in Purification and Analytical Methods herein. Isolated conjugates had greater than 90% purity.

DAR1 Fab siRNA Conjugate Synthesis Using C16 Linker

### Step 1: Synthesis of Fmoc-L-Cys-OC16 Adaptor

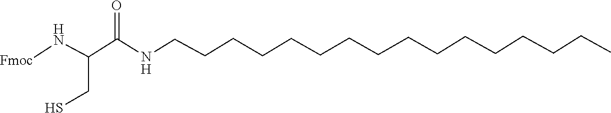

Fmoc-L-Cys(Trt)-OH was purchased from Chem-Impex (Catalog #00177), 1-[Bis(dimethylamino)methylene]-1H-1,2,3-triazolo[4,5-b]pyridinium 3-oxid hexafluorophosphate (HATU) was purchased from CreoSalus (Catalog #RC8110), N,N Diisopropylethylamine (DIPEA) was purchased from TCI America (Catalog #D1599), and Hexadecylamine (C16-amine) was purchased from Sigma Aldrich (Catalog #445312). HATU was dissolved in dichloromethane (DCM), to which was added 1.1 Eq of Fmoc-L-Cys(Trt)-OH, 2 Eq of DIPEA, and 0.6 Eq of C16-amine. The reaction was stirred vigorously for 6 hours and analyzed by thin layer chromatography (TLC) and Reversed-Phase High-Performance Liquid Chromatography Mass Spectrometry (RP-HPLC-MS). The reaction was concentrated on a rotary evaporator, then resuspended in 50/50 Methanol/DCM and was mixed with 20 mass equivalents of celite, then slowly reconcentrated by rotary evaporation. The resulting “dry load” was used to load a silica column and the Fmoc-L-Cys(Trt)-OC16 product was purified by automated flash chromatography. The resulting fractions were pooled and concentrated by rotary evaporation. The residue was resuspended in 90% TFA, 5% triisopropylsilane, and 5% methanol for 10 minutes before being concentrated by rotary evaporation, then resuspended in 50/50 Methanol/DCM and mixed with 20 mass equivalents of celite, then slowly reconcentrated by rotary evaporation. The resulting “dry load” was used to load a silica column and the Fmoc-L-Cys-OC16 product was purified by automated flash chromatography. The pure fractions were pooled and concentrated by rotary evaporation after RP-HPLC-MS analysis and the adaptor was recovered in 70% yield (calculated from input of C16-amine). Stored at −20° C. until reaction with MCC-siRNA duplex.

Step 2: Addition of SMCC Linker to siRNA Duplex

SMCC linker was purchased from ThermoFisher (Catalog #22360) and a stock solution was prepared at 60 mg/ml in dimethyl sulfoxide (DMSO). To duplexed siRNA in deionized water was added 1 M phosphate buffer, pH 7.2 to a final concentration of 50 mM phosphate buffer and the pH adjusted to 7.6. To the duplex was added sufficient DMSO to bring the final concentration DMSO in the reaction to 60% (v/v) and 10 equivalents of the SMCC linker. The reaction was allowed to proceed for 1 hr before dilution to 5% DMSO in cold 10 mM acetate buffer, pH 5. The diluted reaction mixture was purified by spin filtration (5×) using 3K MWCO filters. The linker-modified duplex was analyzed using LCMS and immediately used for Step 3.

### Step 3: Addition of Fmoc-L-Cys-OC16 Adaptor to MCC-siRNA Duplex

Fmoc-L-Cys-OC16 was suspended in DMSO to a concentration of 10 mg/mL. The MCC-siRNA duplex was diluted with an equal volume of 50 mM sodium phosphate pH 7.6, then 30 equivalents of the Fmoc-L-Cys-OC16 stock and enough DMSO to bring the final DMSO concentration to 74% were added to the reaction. The reaction was allowed to proceed for 2 hours at room temperature and 12 hours at 4° C., before being 20-fold diluted with water and filtered through a 0.2 micron PES filter. Spin filtration (5×) using 3K MWCO filters was used to concentrate the material into 10% acetonitrile in water. The modified duplex was analyzed using LCMS and immediately used for Step 4.

Step 4: Deprotection of Fmoc with Simultaneous Maleimide Hydrolysis

To the solution of Fmoc-L-Cys(MCC-siRNA)-OC16 in 10% acetonitrile was added enough 4-methylpiperidine to bring the final concentration of 4-methylpiperidine to 10%. The reaction was allowed to proceed for 2.5 hours before being diluted 10-fold with 50 mM sodium phosphate pH 7.6 and concentrated into the same buffer by spin filtration (5×) using 3K MWCO filters. The modified duplex was analyzed by LCMS to show that Fmoc deprotection and maleimide hydrolysis had both proceeded and stored at −20° C. until reaction with SMCC.

### Step 5: Addition of SMCC Linker to Cys(MCC-siRNA)-OC16

SMCC was purchased from ThermoFisher (Catalog #22360) and a stock solution was prepared at 60 mg/ml in dimethyl sulfoxide (DMSO). To duplexed siRNA in deionized water was added 1 M phosphate buffer, pH 7.2 to a final concentration of 50 mM phosphate buffer and the pH adjusted to 7.6. To the duplex was added sufficient DMSO to bring the final concentration DMSO in the reaction to 65% (v/v) and 10 equivalents of the SMCC linker. The reaction was allowed to proceed for 3 hrs before dilution to 5% DMSO in cold 10 mM acetate buffer, pH 6. The diluted reaction mixture was purified by spin filtration (5×) using 3K MWCO filters. The linker-modified duplex was analyzed using LCMS and stored at −20° C. until reaction with reduced Fab.

Step 6: Fab Interchain Disulfide Reduction with TCEP

To a solution of Fab in phosphate buffered saline (PBS, pH 7.4) at 26.18 mg/ml protein concentration was added sufficient 0.5 M ethylenediaminetetraacetic acid (EDTA) to afford a final concentration of 2 mM EDTA in the final reaction solution. To the solution containing Fab and EDTA was added 1.5 equivalents of tris(2-carboxyethyl)phosphine hydrochloride (TCEP) in water and incubated for 4 hours at 37° C. To the resultant reduced antibody solution was added a solution of SMCC-C6-siRNA (2 equivalents) in 10 mM sodium acetate buffer, pH 6. The reaction proceeded for 16 hours at room temperature before quenching with 10 equivalents of N-ethyl maleimide (NEM) as a 10 mg/ml stock solution in dimethyl sulfoxide (DMSO). Analysis of the reaction mixture by analytical SAX and SEC column chromatography showed Fab-siRNA conjugate along with unreacted Fab and siRNA.

Step 7: Purification of Fab siRNA Conjugate

The crude reaction mixture was purified by AKTA explorer FPLC using anion exchange chromatography method-2. Fractions containing DAR1 and DAR2 conjugates were separated, concentrated and buffer exchanged with pH 7.4 PBS.

### Step 8: Analysis of the Purified Conjugate

The isolated conjugates were characterized by SEC with SEC using size exclusion chromatography method-2 and SAX chromatography anion exchange chromatography method-3 as described in Purification and Analytical Methods section herein. Isolated conjugates had greater than 90% purity.

### DAR1 Fab-siRNA Conjugate Synthesis Using C16 Linker

### Step 1: Synthesis of Mal-Peg12-C16 Capping Agent

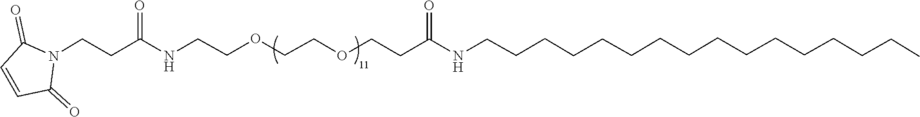

Mal-PEG12-NHS Ester was purchased from Quanta Biosciences (Catalog #10284), N,N Diisopropylethylamine (DIPEA) was purchased from TCI America (Catalog #D1599), and Hexadecylamine (C16-amine) was purchased from Sigma Aldrich (Catalog #445312). Dissolved Mal-PEG12-NHS Ester in dichloromethane (DCM) along with 0.4 Eq of C16-amine and 2 Eq of DIPEA, then allowed to stir for 16 hours before removing DCM by rotary evaporation. Resuspended in 3:1 Ethyl Acetate:Heptanes (organic wash) and added to a separatory funnel, then rinsed the vial with the organic wash and added it to the material in the funnel. Washed the organic layer twice with concentrate aqueous sodium bicarbonate, then eluted the organic fraction and concentrated by rotary evaporation. Resuspended in DCM and loaded onto a silica column for purification by automated flash chromatography. Pooled pure fractions and concentrated by rotary evaporation then analyzed by thin layer chromatography (TLC) and Reversed-Phase High-Performance Liquid Chromatography Mass Spectrometry (RP-HPLC-MS). Stored at 4° C. until reaction with Fab-siRNA conjugate.

Step 2: Addition of SMCC Linker to siRNA Duplex

SMCC was purchased from ThermoFisher (Catalog #22360) and a stock solution was prepared at 60 mg/ml in dimethyl sulfoxide (DMSO). To duplexed siRNA in deionized water was added 1 M phosphate buffer, pH 7.2 to a final concentration of 50 mM phosphate buffer and the pH adjusted to 7.6. To the duplex was added sufficient DMSO to bring the final concentration DMSO in the reaction to 60% (v/v) and 10 equivalents of the SMCC linker. The reaction was allowed to proceed for 1 hr before dilution to 5% DMSO in cold 10 mM acetate buffer, pH 6. The diluted reaction mixture was purified by spin filtration (5×) using 3K MWCO filters. The linker-modified duplex was analyzed using LCMS and stored at −20° C. until reaction with reduced antibody.

Step 3: Fab Interchain Disulfide Reduction with TCEP

To a solution of Fab in phosphate buffered saline (PBS, pH 7.4) at 26.18 mg/ml protein concentration was added sufficient 0.5 M ethylenediaminetetraacetic acid (EDTA) to afford a final concentration of 2 mM EDTA in the final reaction solution. To the solution containing Fab and EDTA was added 1.5 equivalents of tris(2-carboxyethyl)phosphine hydrochloride (TCEP) in water and incubated for 4 hours at 37° C. To the resultant reduced antibody solution was added a solution of SMCC-C6-siRNA (2 equivalents) in 10 mM sodium acetate buffer, pH 6. The reaction proceeded for 16 hours at room temperature before quenching with 20 equivalents of Mal-PEG12-C16 as a 50 mg/ml stock solution in dimethyl sulfoxide (DMSO) for 90 minutes. Analysis of the reaction mixture by analytical SAX and SEC column chromatography showed Fab siRNA conjugate along with unreacted Fab and siRNA.

Step 4: Purification of Fab siRNA Conjugate

The crude reaction mixture was purified by AKTA explorer FPLC using anion exchange chromatography method-2. Fractions containing DAR1 and DAR2 conjugates were separated, concentrated and buffer exchanged with pH 7.4 PBS.

### Step 5: Analysis of the Purified Conjugate

The isolated conjugates were characterized by SEC with SEC using size exclusion chromatography method-2 and SAX chromatography anion exchange chromatography method-3 as described in the Purification and Analytical Methods section herein. Isolated conjugates had greater than 90% purity.

### Purification and Analytical Methods

### Anion Exchange Chromatography Method (SAX)-1

1. Column: Sartobind Q 3 mL
2. Solvent A: 20 mM PB pH 7.6; Solvent B: 20 mM PB pH 7.6+1.5 M NaCl; Flow Rate: 8 ml/min

| % A | % B | CV |
|---|---|---|
| 80 | 20 | 1 |
| 50 | 50 | 18 |
| 0 | 100 | 1 |

### Anion Exchange Chromatography Method (SAX)-2

1. Column: SkillPak Toyopearl GigaCap Q-650S 5 mL
2. Solvent A: 20 mM PB pH 7.6; Solvent B: 20 mM PB pH 7.6+1.5 M NaCl; Flow Rate: 8 ml/min

| % A | % B | CV |
|---|---|---|
| 70 | 30 | 1 |
| 45 | 55 | 20 |
| 90 | 10 | 1 |

### Anion Exchange Chromatography Method (SAX)-3

1. Column: Thermo Scientific, Pro pac SAX-10, BioLC, 4×250 mm
2. Solvent A: 80% 10 mM Tris pH 7.2, 20% ethanol; Solvent B: 80% 10 mM Tris pH 7.2, 20% ethanol, 1.5 M NaCl; Flow Rate: 0.92 ml/min

| Time | % A | % B |
|---|---|---|
| 0 | 84 | 16 |
| 2 | 60 | 40 |
| 4 | 46.5 | 53.5 |
| 4.5 | 84 | 16 |
| 5.5 | 84 | 16 |

### Anion Exchange Chromatography Method (SAX)-4

1. Column: Thermo Scientific, Pro pac SAX-10, BioLC, 4×250 mm
2. Solvent A: 80% 10 mM Tris pH 7.2, 20% ethanol; Solvent B: 80% 10 mM Tris pH 7.2, 20% ethanol, 1.5 M NaCl; Flow Rate: 0.75 ml/min

| Time | % A | % B |
|---|---|---|
| 0 | 90 | 10 |
| 2.5 | 90 | 10 |
| 20.5 | 25 | 75 |
| 21.5 | 0 | 100 |
| 22 | 90 | 10 |
| 25 | 90 | 10 |

### Anion Exchange Chromatography Method (SAX)-5

1. Column: Thermo Scientific, Pro pac SAX-10, BioLC, 4×250 mm
2. Solvent A: 80% 10 mM Tris pH 7.2, 20% ethanol; Solvent B: 80% 10 mM Tris pH 7.2, 20% ethanol, 1.5 M NaCl; Flow Rate: 0.75 ml/min

| Time | % A | % B |
|---|---|---|
| 0 | 90 | 10 |
| 3 | 90 | 10 |
| 11 | 40 | 60 |
| 11.5 | 90 | 10 |
| 12.5 | 90 | 10 |

### Size Exclusion Chromatography Method (SEC)-1

1. Column: Bioresolve SEC mAb 200A 2.5 um 4.6×150 mm
2. Solvent A: 200 mM sodium phosphate pH 7.0, 10% Acetonitrile; Flow Rate: 0.4 ml/min

| Time | % A |
|---|---|
| 0 | 100 |
| 5.5 | 100 |

### Size Exclusion Chromatography Method (SEC)-2

1. Column: Bioresolve SEC mAb 200A 2.5 um 7.8×300 mm
2. Solvent A: 50 mM potassium phosphate, 200 mM potassium chloride, pH 6.8+10% isopropanol; Flow Rate: 0.5 ml/min

| Time | % A |
|---|---|
| 0 | 100 |
| 30 | 100 |

### Animals

All animal studies were conducted following protocols in accordance with the Institutional Animal Care and Use Committee (IACUC) at Explora BioLabs, which adhere to the regulations outlined in the USDA Animal Welfare Act as well as the “Guide for the Care and Use of Laboratory Animals” (National Research Council publication, 8th Ed., revised in 2011). All mice were obtained from either Charles River Laboratories, Harlan Laboratories, or BioCytogen.

Wild type CD-1 mice (5-6 weeks old) were dosed as indicated via intravenous (IV) or subcutaneous (SC) injection with the indicated nucleic acid conjugate and doses. Humanized transferrin receptor 1 C57BL/6-Tfrc^{tm1(TFRC)Bcgen}/Bcgen mice were obtained from Biocytogen.

In Vitro Evaluation of siRNA Potency and Efficacy

C2C12 myoblasts (ATCC) were grown in DMEM supplemented with 10% v/v FBS. For transfection, cells were plated at a density of 10,000 cells/well in 24-well plates, and transfected within 24 hours. C2C12 myotubes were generated by incubating confluent C2C12 myoblast cultures in DMEM supplemented with 2% v/v horse serum for 3-4 days. During and after differentiation the medium was changed daily. Pre-differentiated primary human skeletal muscle cells were obtained from ThermoFisher and plated in DMEM with 2% v/v horse serum according to recommendations by the manufacturer. Human SJCRH30 rhabdomyosarcoma myoblasts (ATCC) were grown in DMEM supplemented with 10% v/v heat-inactivated fetal calf serum, 4.5 mg/mL glucose, 4 mM L-glutamine, 10 mM HEPES, and 1 mM sodium pyruvate. For transfections cells were plated in a density of 10,000-20,000 cells/well in 24-well plates and transfected within 24 hours. All cells were transfected with various concentrations of the siRNAs (0.0001-100 nM; 10-fold dilutions) using RNAiMax (ThermoFisher) according to the recommendation by the manufacturer. Transfected cells were incubated in 5% CO₂ at 37° C. for 2 days, then washed with PBS, and harvested in 300 ul TRIzol (ThermoFisher) and stored at −80° C. RNA was prepared using a ZYMO 96-well RNA kit (ThermoFisher) and relative RNA expression levels quantified by RT-qPCR using commercially available TaqMan probes (LifeTechnology). Expression data were analyzed using the ΔΔCT method normalized to Ppib expression, and are presented as % KD relative to mock-transfected cells. Data were analyzed by nonlinear regression using a 3-parameter dose response inhibition function (GraphPad Prism 7.02). All knock down results present the maximal observed KD under these experimental conditions.

### RISC Loading Assay

Specific immunoprecipitation of the RISC from tissue lysates and quantification of small RNAs in the immunoprecipitates were determined by stem-loop PCR, using an adaptation of the assay described by Pei et al. Quantitative evaluation of siRNA delivery in vivo. RNA (2010), 16:2553-2563.

### CE-SDS Analysis

Quantitative capillary electrophoresis-sodium dodecyl sulphate (CE-SDS) under reducing conditions was performed according to instrument specifications on a Biotechne ProteinSimple Maurice instrument (Catalog #090-000) and CE-SDS Plus Cartridge (Catalog #PS-MC02-SP). All data was analyzed with Compass for iCE software and JMP.

### Fluorescence Polarization Assay

mAb siRNA DAR1 conjugates were labelled at the 3′ terminus of the passenger strand with Alexa Fluor 647 Dye. Binding measurements were conducted in 1×DPBS (Gibco) in flat-bottom non-binding 96-well plates (Corning, NY, USA) at 25° C. ALEXA647-labelled conjugates were added at a final concentration of 3 nM to solutions of HSA with ranging concentrations. Fluorescence polarization (λex=620 nm, λem=680 nm) was measured on a Tecan Spark (Baldwin Park, CA, USA). Data analysis was performed using GraphPad Prism 10.2.2 software (GraphPad Software, La Jolla, CA, USA) using non-linear regression for curve fit assuming one binding site.

### Example 4: Duration of Action of mTfR1-mAb-pos6.C16.siMSTN DAR1 in CD1 Mice

MSTN: A 21-mer double-stranded siRNA with 19 bases of complementarity and 3′ dinucleotide overhangs was designed against mouse MSTN. The sequence (5′ to 3′) of the guide/antisense strand was UUAUUAUUUGUUCUUUGCCUU (SEQ ID NO: 4). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker.

### Conjugate Synthesis and Characterization

The mouse transferrin 1 receptor (mTfR1) mAb-MSTN siRNA (DAR1 conjugates were made, purified and characterized as described in Example 3.1.1. mTfR1-mAb-pos6.C16.siMSTN DAR1 is a mouse transferrin 1 receptor (mTfR1) mAb-MSTN siRNA conjugate, which sense/passenger strand is conjugated with C16 hydrocarbon chain at the nucleotide of position 6 from the 5′ end. All conjugates were made through cysteine conjugation using a bismal linker. Conjugates were characterized chromatographically as described in Table 3.

TABLE 3
HPLC Retention Time (RT) in Minutes

| | | RT, SAX | RT, SEC |
|---|---|---|---|
| Group(s) | Conjugate | Method-3 | Method-1 |
| 1-3 | mTfR1-mAb-siMSTN DAR1 | 3.478 | 2.941 |
| 4-6 | mTfR1-mAb-pos6.C16.siMSTN | 3.437 | 3.476 |
| | DAR1 | | |

### In Vivo Study Design

The conjugates were assessed for their ability to mediate mRNA downregulation of myostatin (MSTN) in skeletal muscle in vivo in wild type CD-1 mice. Mice were dosed via intravenous (IV) injection with PBS vehicle control or the indicated doses shown in Table 4. After 28, 56, or 84 days, gastrocnemius (gastroc) and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 4
In vivo study design testing duration of action of mTfR1-bismal-
pos.6.C16.siMSTN DAR1 in CD1 Mice

| | | | | | | | Inj | Inj | |
|---|---|---|---|---|---|---|---|---|---|
| | | | | siRNA | mAb | siRNA | Vol | Vol | |
| | | | | Conc | Conc | Dose | per | per | |
| | | siRNA | | (mg/ | (mg/ | (mg/ | Animal | Group | Harvest |
| Group(s) | Test Article | Target | N | mL) | ml) | kg) | (mL) | (mL) | (day) |
| 1 | mTfR1-bismal- | MSTN | 4 | 0.15 | 1.61 | 0.75 | 0.175 | 0.7 | 28 |
| | siMSTN DAR1 | | | | | | | | |
| 2 | mTfR1-bismal- | MSTN | 4 | 0.15 | 1.61 | 0.75 | 0.175 | 0.7 | 56 |
| | siMSTN DAR1 | | | | | | | | |
| 3 | mTfR1-bismal- | MSTN | 4 | 0.15 | 1.61 | 0.75 | 0.175 | 0.7 | 84 |
| | siMSTN DAR1 | | | | | | | | |
| 4 | mTfR 1-bismal- | MSTN | 4 | 0.15 | 1.61 | 0.75 | 0.175 | 0.7 | 28 |
| | pos.6.C16.siMSTN | | | | | | | | |
| | DAR1 | | | | | | | | |
| 5 | mTfR 1-bismal- | MSTN | 4 | 0.15 | 1.61 | 0.75 | 0.175 | 0.7 | 56 |
| | pos.6.C16.siMSTN | | | | | | | | |
| | DAR1 | | | | | | | | |
| 6 | mTfR1-bismal- | MSTN | 4 | 0.15 | 1.61 | 0.75 | 0.175 | 0.7 | 84 |
| | pos.6.C16.siMSTN | | | | | | | | |
| | DAR1 | | | | | | | | |
| 7 | PBS | N/A | 4 | N/A | N/A | N/A | 0.175 | 0.7 | 28 |
| 8 | PBS | N/A | 4 | N/A | N/A | N/A | 0.175 | 0.7 | 56 |
| 9 | PBS | N/A | 4 | N/A | N/A | N/A | 0.175 | 0.7 | 84 |

* All animals used in experiments shown in Table 4 have body weight of 0.035 kg, and the injection was made at a single dose volume of 5 ml/kg by IV administration.

TABLE 5
siRNA Sequences used in Example 4

| | | SEQ | | |
|---|---|---|---|---|
| SIRNA | | ID | | SEQ ID |
| Ref. | siRNA Guide Sequence | NO: | siRNA Passenger Sequence | NO: |
| siDMPK | [5vp_una2moe](U)[ps].[fl2r](A)[ps].m | 9 | [5am6_p_m](C)[ps].m(C)[ps].m(G) | 46 |
| | (A)p.m(U)p.m(A)p.[fl2r](C)p.m(C) | | p.m(A)p.m(C)p.m(A)p.[fl2r](U) | |
| | p.m(G)p.m(A)p.m(G)p.m(G)p.m(A) | | p.[fl2r](U)p.[fl2r](C)p.m(C) | |
| | p.m(A)p.[fl2r](U)p.m(G)p.[fl2r](U) | | p.m(U)p.m(C)p.m(G)p.m(G)p.m(U) | |
| | p.m(C)p.m(G)p.m(G)[ps].m(U)[ps].m | | p.m(A)p.m(U)[ps].m(U)[ps].m(A) | |
| | (U) | | | |
| pos6.C16. | [5vp_una2moe](U)[ps].[fl2r](A)[ps].m | 10 | [5am6_p_m](C)[ps].m(C)[ps].m(G) | 47 |
| siDMPK | (A)p.m(U)p.m(A)p.[fl2r](C) | | p.m(A)p.m(C)p.[rc16](A)p.[fl2r](U) | |
| | p.m(C)p.m(G)p.m(A)p.m(G)p.m(G) | | p.[fl2r](U)p.[fl2r](C)p.m(C) | |
| | p.m(A)p.m(A)p.[fl2r](U)p.m(G)p.[fl2r] | | p.m(U)p.m(C)p.m(G)p.m(G)p.m(U) | |
| | (U)p.m(C)p.m(G)p.m(G)[ps].m(U) | | p.m(A)p.m(U)[ps].m(U)[ps].m(A) | |
| | [ps].m(U) | | | |

5vp_una2moe 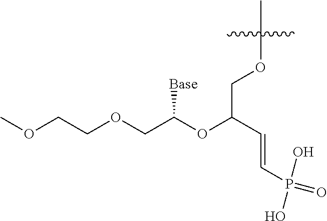
5am6_p_m 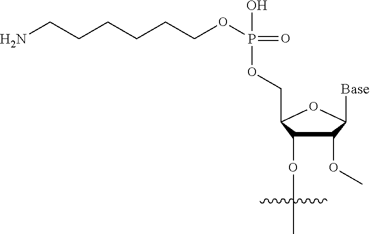
rc16 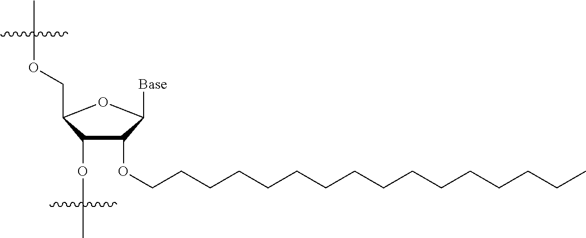
5vp_una2moe 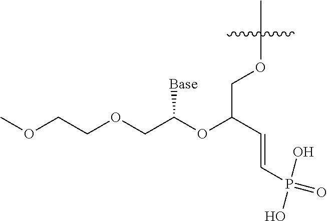
5am6_p_m 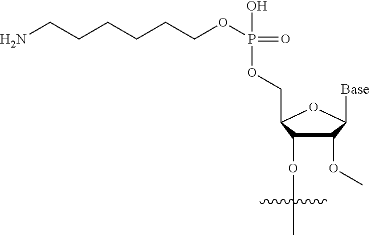
rc16

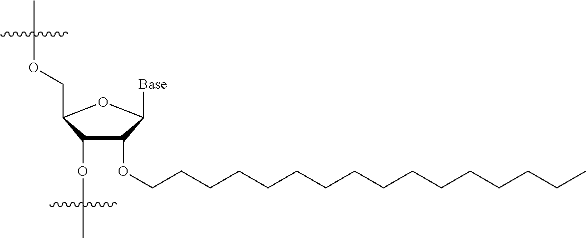

In Table 5, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

### Results

MSTN siRNA tissue concentrations and MSTN mRNA expression levels were assessed with DAR1 lipid AOCs in a time dependent manner in the gastroc or heart tissue obtained from mice following a single intravenous injection of a DAR1 control AOC (mTfR1-bismal-siMSTN) or the DAR1 lipid AOC with C16 hydrocarbon chain (C16 lipid) at position 6 (from 5′ end) of the passenger strand of the siRNA (mTfR1-bismal.pos6.C16.siMSTN) at the dose shown in Table 4 at Day 0.

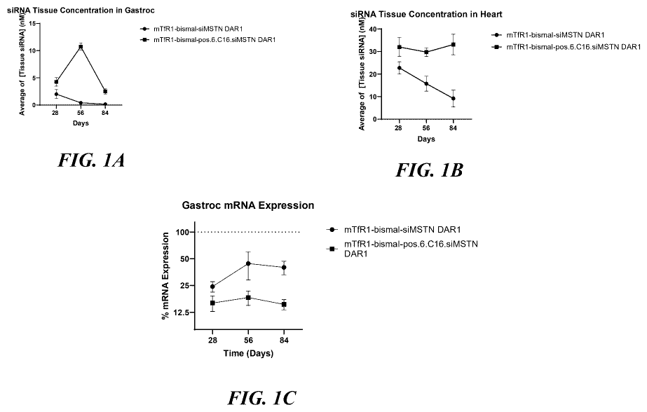

Lipid AOCs deliver higher concentrations of MSTN siRNA in the gastroc and heart than the control AOCs over the 84-day period (FIGS. 1A-B). At day 84 the lipid AOCs has a 3 to 4-fold increase in MSTN siRNA tissue concentrations in the gastroc and heart compared to control AOCs. In addition, the lipid AOCs has increased reduction in MSTN mRNA expression levels gastroc compared to the levels of control AOCs (FIG. 1C). At day 84, lipid AOCs reduced mRNA expression levels by 85% (15% mRNA expression level) while the control AOCs reduced MSTN mRNA expression levels by 55% (45% mRNA expression level), which indicates that the lipid AOCs could show a 3-fold increased activity compared to the control AOCs.

Overall, the lipid AOCs with the C16 lipid attached to the position 6 nucleotide of the passenger strand of the MSTN siRNA provide higher delivery and retention of MSTN siRNA in tissues and enhanced reduction in MSTN mRNA expression levels sustained over an extended period.

### Example 5: Duration of Action of a 2′-O—C16 DMPK siRNA mAb DAR1 AOC in CD1 Mice

DMPK: A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker.

### Conjugate Synthesis and Characterization

The CD71 mAb-siRNA DAR1 conjugates were made, purified and characterized as described in Example 3. mTfR1-mAb-pos6.C16.siDMPK is a mouse transferrin 1 receptor (mTfR1) mAb-DMPK siRNA conjugate, which sense/passenger strand is conjugated with C16 hydrocarbon chain at the nucleotide of position 6 from the 5′ end. mIsotype-mAb-siDMPK is a mouse IgG2a (control) antibody-DMPK siRNA conjugate. mIsotype-mAb-pos6.C16.siDMPK is a mouse IgG2a (control) antibody-DMPK siRNA conjugate, which sense/passenger strand is conjugated with C16 hydrocarbon chain at the nucleotide of position 6 from the 5′ end. All conjugates were made through cysteine conjugation, a bismal linker. Conjugates were characterized chromatographically as described in Table 6.

TABLE 6
HPLC Retention Time (RT) in Minutes

| | | RT, SAX | RT, SEC |
|---|---|---|---|
| Group(s) | Conjugate | Method-3 | Method-1 |
| 1-5 | mTfR1-mAb-siDMPK DAR1 | 3.197 | 3.037 |
| 6-10 | mTfR1-mAb-pos6.C16.siDMPK | 3.211 | 3.098 |
| | DAR1 | | |
| 11-12 | mIsotype-mAb-siDMPK DAR1 | 3.233 | 2.953 |
| 13-14 | mIsotype-mAb-pos6.C16.siDMPK | 3.251 | 2.989 |
| | DAR1 | | |

### In Vivo Study Design

The conjugates were assessed for their ability to mediate mRNA downregulation of myotonic dystrophy protein kinase (DMPK) in skeletal muscle in vivo in wild type CD-1 mice. Mice were dosed via intravenous (IV) injection with PBS vehicle control or the indicated ASCs and at the indicated dose as shown in Table 7. After 28, 56, or 84 days, gastrocnemius (gastroc) and heart muscle tissues were harvested and snap-frozen in liquid nitrogen. mRNA knockdown in target tissue was determined using a comparative (PCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 7
In vivo study design testing duration of action of a 2′-O-C16 DMPK siRNA mAb DAR1
AOC in CD1 Mice.

| | | | | siRNA: | | | | |
|---|---|---|---|---|---|---|---|---|
| | | | | mAb | siRNA | siRNA | | |
| | | | | Ratio | Conc | Dose | | |
| | | siRNA | | (mol/ | (mg/ | (mg/ | Doses | Harvest |
| Group | Test Article | Target | N | mol) | mL) | kg) | (#) | (day) |
| 1 | mTfR1-mAb- | DMPK | 4 | 1 | 0.15 | 0.75 | 1 | 1 d |
| | siDMPK DAR1 | | | | | | | |
| 2 | mTfR1-mAb- | DMPK | 4 | 1 | 0.15 | 0.75 | 1 | 14 d |
| | siDMPK DAR1 | | | | | | | |
| 3 | mTfR1-mAb- | DMPK | 4 | 1 | 0.15 | 0.75 | 1 | 28 d |
| | siDMPK DAR1 | | | | | | | |
| 4 | mTfR1-mAb- | DMPK | 4 | 1 | 0.15 | 0.75 | 1 | 56 d |
| | siDMPK DAR1 | | | | | | | |
| 5 | mTfR1-mAb- | DMPK | 4 | 1 | 0.15 | 0.75 | 1 | 84 d |
| | siDMPK DAR1 | | | | | | | |
| 6 | mTfR1-mAb- | DMPK | 4 | 1 | 0.15 | 0.75 | 1 | 1 d |
| | pos6.C16.siDMPK | | | | | | | |
| | DAR1 | | | | | | | |
| 7 | mTfR1-mAb- | DMPK | 4 | 1 | 0.15 | 0.75 | 1 | 14 d |
| | pos6.C16.siDMPK | | | | | | | |
| | DAR1 | | | | | | | |
| 8 | mTfR1-mAb- | DMPK | 4 | 1 | 0.15 | 0.75 | 1 | 28 d |
| | pos6.C16.siDMPK | | | | | | | |
| | DAR1 | | | | | | | |
| 9 | mTfR1-mAb- | DMPK | 4 | 1 | 0.15 | 0.75 | 1 | 56 d |
| | pos6.C16.siDMPK | | | | | | | |
| | DAR1 | | | | | | | |
| 10 | mTfR1-mAb- | DMPK | 4 | 1 | 0.15 | 0.75 | 1 | 84 d |
| | pos6.C16.siDMPK | | | | | | | |
| | DAR1 | | | | | | | |
| 11 | mIsotype-mAb- | DMPK | 4 | 1 | 0.15 | 0.75 | 1 | 1 d |
| | siDMPK DAR1 | | | | | | | |
| 12 | mIsotype-mAb- | DMPK | 4 | 1 | 0.15 | 0.75 | 1 | 28 d |
| | siDMPK DAR1 | | | | | | | |
| 13 | mIsotype-mAb- | DMPK | 4 | 1 | 0.15 | 0.75 | 1 | 1 d |
| | pos6.C16.siDMPK | | | | | | | |
| | DAR1 | | | | | | | |
| 14 | mIsotype-mAb- | DMPK | 4 | 1 | 0.15 | 0.75 | 1 | 28 d |
| | pos6.C16.siDMPK | | | | | | | |
| | DAR1 | | | | | | | |
| 15 | PBS | N/A | 4 | 1 | N/A | N/A | 1 | 14 d |
| 16 | PBS | N/A | 4 | 1 | N/A | N/A | 1 | 28 d |
| 17 | PBS | N/A | 4 | 1 | N/A | N/A | 1 | 56 d |
| 18 | PBS | N/A | 4 | 1 | N/A | N/A | 1 | 84 d |

* All animals used in experiments shown in Table 7 had injections at a single dose volume of 5 ml/kg by IV administration.

TABLE 8
siRNA Sequences used in Example 5

| | | | SEQ | | SEQ |
|---|---|---|---|---|---|
| Compound | siRNA | | ID | | ID |
| No. | Ref. | siRNA Guide Sequence | NO: | siRNA Passenger Sequence | NO: |
| 1 | siDMP | [5vp_una2moe](U)[ps].[fl2r](A) | 11 | [5am6_p_m](C)[ps].m(C)[ps].m | 48 |
| | K | [ps].m(A)p.m(U)p.m(A)p.[fl2r] | | (G)p.m(A)p.m(C)p.m(A)p.[fl2r] | |
| | | (C)p.m(C)p.m(G)p.m(A)p.m(G) | | (U)p.[fl2r](U)p.[fl2r](C)p.m(C) | |
| | | p.m(G)p.m(A)p.m(A)p.[fl2r](U) | | p.m(U)p.m(C)p.m(G)p.m(G)p.m | |
| | | p.m(G)p.[fl2r](U)p.m(C)p.m(G) | | (U)p.m(A)p.m(U)[ps].m(U)[ps].m | |
| | | p.m(G)[ps].m(U)[ps].m(U)} | | (A) | |
| 2 | pos6.C | [5vp_una2moe](U)[ps].[fl2r](A) | 12 | [5am6_p_m](C)[ps].m(C)[ps].m | 49 |
| | 16.siD | [ps].m(A)p.m(U)p.m(A)p.[fl2r] | | (G)p.m(A)p.m(C)p.[rc16](A) | |
| | MPK | (C)p.m(C)p.m(G)p.m(A)p.m(G) | | p.[fl2r](U)p.[fl2r](U)p.[fl2r](C)p.m | |
| | | p.m(G)p.m(A)p.m(A)p.[fl2r](U) | | (C)p.m(U)p.m(C)p.m(G)p.m(G) | |
| | | p.m(G)p.[fl2r](U)p.m(C)p.m(G) | | p.m(U)p.m(A)p.m(U)[ps].m(U) | |
| | | p.m(G)[ps].m(U)[ps].m(U) | | [ps].m(A) | |

5vp_una2moe 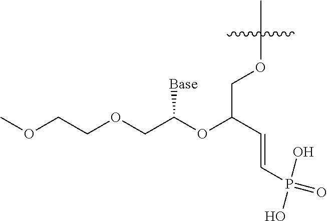
5am6_p_m 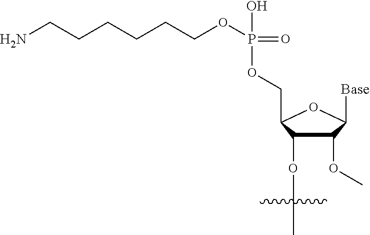
rc16

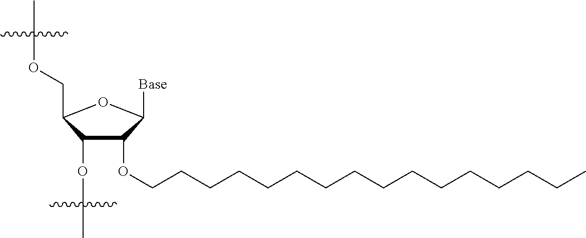

In Table 8, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

TABLE 9
Bₘₐₓ and K_{d} of the mTfR1-mAb-pos6.C16.siDMPK DAR1

| Conjugate | Bₘₐₓ | h | K_{d} |
|---|---|---|---|
| mTfR1-mAb-siDMPK DAR1 | 3.851 | 1.211 | 68.28 |
| mTfR1-mAb-pos6.C16.siDMPK DAR1 | 3.851 | 1.21 | 68.28 |
| mIsotype-mAb-siDMPK DAR1 | 48.26 | 1.27 | unstable |
| mIsotype-mAb-pos6.C16.siDMPK DAR1 | 0.3622 | 0.7328 | 116593 |
| mTfR1-mAb | 3.614 | 1.194 | 106.3 |
| mIsotype-mAb | 0.1054 | 8.924 | 3923 |

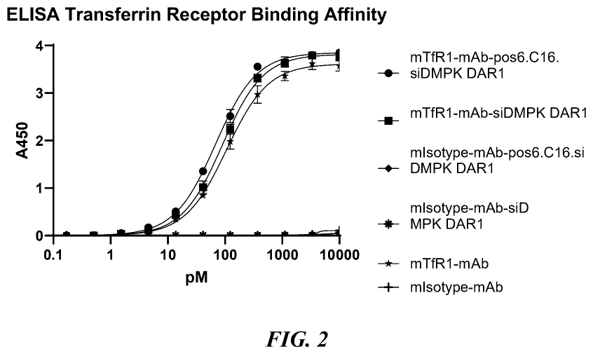

As shown in FIG. 2 and Table 9, the ELISA binding affinity shows that the binding affinity of the lipid AOC with the C16 lipid attached to the passenger strand at position 6 (mTfR1-mAb.pos6.C16.siDMPK) is the same as the control AOC, the monoclonal antibody conjugated with the DMPK siRNA (mTfR1-mAb.siDMPK). The results indicate the addition of the lipid at position 6 nucleotide of the passenger strand of the siRNA does not affect the receptor binding affinity of the antibody of the lipid AOC.

In addition, DMPK siRNA tissue concentrations and DMPK mRNA expression levels were assessed with DAR1 lipid AOC (mTfR1-mAb.pos6.C16.siDMPK) in a time dependent manner in the gastroc, tibialis anterior (TA), heart, or liver tissue obtained from mice following a single injection of a DAR1 control AOC (mTfR1-mAb-siDMPK) or the DAR1 lipid AOC with C16 lipid at position 6 nucleotide of the passenger strand of the siRNA (mTfR1-mAb-pos6.C16.siDMPK) at Day 0.

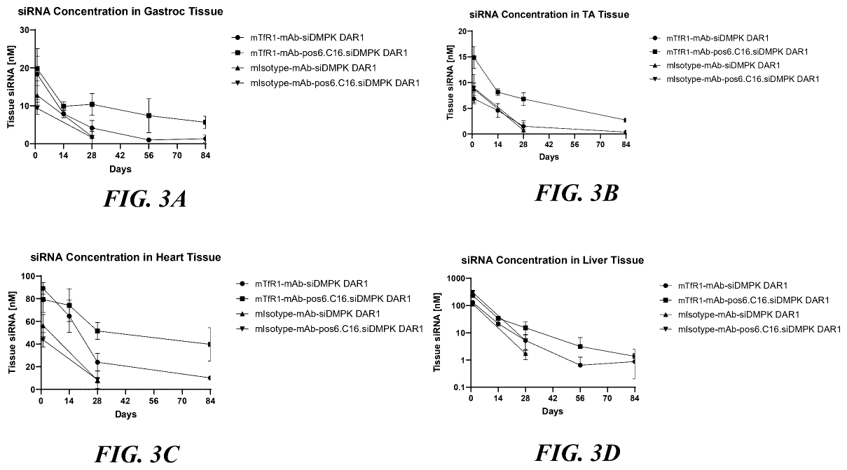

As shown in FIGS. 3A-D, lipid AOCs (mTfR1-mAb-pos6.C16.siDMPK) delivered higher concentrations of DMPK siRNA in the gastroc, TA, heart, and liver than the control AOCs over the 84-day period. The largest differences in DMPK siRNA tissue concentrations between the lipid AOC and control AOCs were observed in the heart at day 28 and day 84 (FIG. 3C) while the smallest differences in DMPK siRNA tissue concentrations were observed in the liver tissue. At day 84 the lipid AOCs has about a 4-fold increase in siRNA tissue concentrations in the heart tissue compared to the control AOCs.

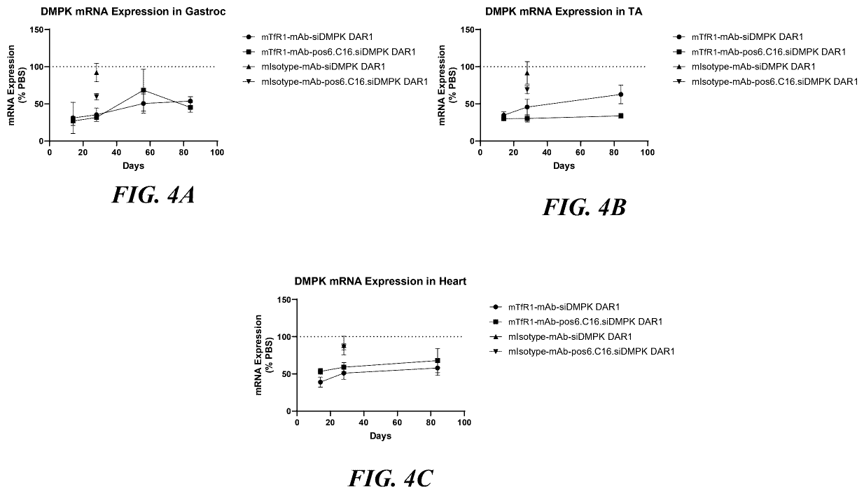

Moreover, the lipid AOCs increased reduction in DMPK mRNA expression levels in TA muscle tissue compared to the levels of control AOCs (FIG. 4B). At day 84, lipid AOCs reduced mRNA expression levels by 75% (25% mRNA expression) in TA while the control AOCs reduced mRNA expression levels by 45% (55% mRNA expression), which indicates a 2-fold increased activity of lipid-AOCs compared to the control AOCs. The differences in DMPK mRNA expression levels between the lipid AOCs or the control AOCs for the gastroc and heart tissues were not significant in this study (FIG. 4A and FIG. 4C).

Overall, the lipid AOCs with the C16 lipid attached to the position 6 of passenger strand of the DMPK siRNA provide higher DMPK siRNA tissue concentrations in skeletal and cardiac muscle tissues and improved reduction in DMPK mRNA expression levels in skeletal muscles over an extended period of time.

### Example 6. Passenger Strand C16 Location Duration of Action in CD1 Mice

SSB: A 21-mer double-stranded siRNA with 19 bases of complementarity and 3′ dinucleotide overhangs was designed against mouse SSB. The nucleic acid sequence (5′ to 3′) of the guide/antisense strand was UUACAUUAAAGUCUGUUGUUU (SEQ ID NO: 5). DMPK: A 21-mer duplex with 19 bases of complementarity and 3′ dinucleotide overhangs was designed against mouse DMPK. The nucleic acid sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker.

### Conjugate Synthesis and Characterization

The CD71 mAb-siRNA DAR1 conjugates were made, purified and characterized as described in Examples 1-3. All conjugates were made through cysteine conjugation using an MBS linker. Conjugates were characterized chromatographically as described in Table 10. The lipid-AOCs described in Table 10 have a C16 hydrocarbon conjugated to the sense strand/passenger strand at positions 6, 3, 17, or 19, respectively, from the 5′ end.

TABLE 10
HPLC Retention Time (RT) in Minutes

| | | RT, SAX | RT, SEC |
|---|---|---|---|
| Group(s) | Conjugate | Method-3 | Method-1 |
| 1-2 | mTfR1-mAb-siSSB DAR1 | 3.090 | 2.870 |
| 3-4 | mTfR1-mAb-pos6.C16.siDMPK | 3.114 | 2.913 |
| | DAR1 | | |
| 5-6 | mTfR1-mAb-pos3.C16.siDMPK | 3.072 | 2.883 |
| | DAR1 | | |
| 7-8 | mTfR1-mAb-pos17.C16.siDMPK | 3.154 | 2.982 |
| | DAR1 | | |
| 9-10 | mTfR1-mAb-pos19.C16.siDMPK | 3.221 | 3.087 |
| | DAR1 | | |

### In Vivo Study Design

The conjugates were assessed for their ability to mediate mRNA downregulation of SSB in skeletal muscle in vivo in wild type CD-1 mice. Mice were dosed via intravenous (IV) injection with PBS vehicle control or the indicated ASCs and at 0.5 mg/kg siRNA dose as shown in Table 11. After 28 days, gastrocnemius (gastroc), tibialis anterior (TA), liver, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 11
In vivo study design testing duration of action of SSB siRNA mAb DAR1 AOCs containing 2′
lipid modifications at passenger strand position 3 or 6 in CD1 Mice

| | | | siRNA:mAb | siRNA | | |
|---|---|---|---|---|---|---|
| | | | Ratio | Dose | Doses | Harvest |
| Group(s) | Test Article | N | (mol/mol) | (mg/kg) | (#) | (day) |
| 1 | mTfR1-mAb-siSSB DAR1 | 4 | 1 | 0.5 | 1 | 28 |
| 2 | mTfR1-mAb-siSSB DAR1 | 4 | 1 | 0.5 | 1 | 56 |
| 3 | mTfR1-mAb- | 4 | 1 | 0.5 | 1 | 28 |
| | pos6.C16.siDMPK DAR1 | | | | | |
| 4 | mTfR1-mAb- | 4 | 1 | 0.5 | 1 | 56 |
| | pos6.C16.siDMPK DAR1 | | | | | |
| 5 | mTfR1-mAb- | 4 | 1 | 0.5 | 1 | 28 |
| | pos3.C16.siDMPK DAR1 | | | | | |
| 6 | mTfR1-mAb- | 4 | 1 | 0.5 | 1 | 56 |
| | pos3.C16.siDMPK DAR1 | | | | | |
| 7 | mTfR1-mAb- | 4 | 1 | 0.5 | 1 | 28 |
| | pos 17.C16.siDMPK DAR1 | | | | | |
| 8 | mTfR1-mAb- | 4 | 1 | 0.5 | 1 | 56 |
| | pos 17.C16.siDMPK DAR1 | | | | | |
| 9 | mTfR1-mAb- | 4 | 1 | 0.5 | 1 | 28 |
| | pos 19.C16.siDMPK DAR1 | | | | | |
| 10 | mTfR1-mAb- | 4 | 1 | 0.5 | 1 | 56 |
| | pos 19.C16.siDMPK DAR1 | | | | | |
| 11 | PBS | 4 | 1 | N/A | 1 | 28 |
| 12 | PBS | 4 | 1 | N/A | 1 | 56 |

TABLE 12
siRNA Sequences used in Example 6, Lipid Length Optimization of 2′-O-Lipids
at Passenger Strand Position 3 and Position 6 on siRNA DARI mAb AOCs in CD1 Mice.

| | | | SEQ | | SEQ |
|---|---|---|---|---|---|
| Compound | SiRNA | | ID | | ID |
| No. | Ref. | siRNA Guide Sequence | NO | siRNA Passenger Sequence | NO: |
| 1 | siSSB | [5vp_una2moe](U)[ps].[fl2r] | 13 | [5am6_p_m](A)[ps].m(C)[ps].m | 50 |
| | | (U)[ps].m(A)p.m(C)p.m(A)p. | | (A)p.m(A)p.m(C)p.m(A)p.[fl2r] | |
| | | [fl2r](U)p.m(U)p.m(A)p.m(A) | | (G)p.[fl2r](A)p.[fl2r](C)p.m | |
| | | p.m(A)p.m(G)p.m(U)p.m(C)p. | | (U)p.m(U)p.m(U)p.m(A)p.m(A) | |
| | | [fl2r](U)p.m(G)p.[fl2r](U)p.m | | p.m(U)p.m(G)p.m(U)[ps].m(A) | |
| | | (U)p.m(G)p.m(U)[ps].m(U) | | [ps].m(A) | |
| | | [ps].m(U) | | | |
| 2 | pos6.C16. | [5vp_una2moe](U)[ps].[fl2r] | 13 | [5am6_p_m](A)[ps].m(C)[ps].m | 51 |
| | siSSB | (U)[ps].m(A)p.m(C)p.m(A) | | (A)p.m(A)p.m(C)p.[rc16](A) | |
| | | p.[fl2r](U)p.m(U)p.m(A)p.m(A) | | p.[fl2r](G)p.[fl2r](A)p.[fl2r](C) | |
| | | p.m(A)p.m(G)p.m(U)p.m(C)p. | | p.m(U)p.m(U)p.m(U)p.m(A) | |
| | | [fl2r](U)p.m(G)p.[fl2r](U)p.m | | p.m(A)p.m(U)p.m(G)p.m(U) | |
| | | (U)p.m(G)p.m(U)[ps].m(U)[ps].m | | [ps].m(A)[ps].m(A) | |
| | | (U) | | | |
| 3 | pos3.C16. | [5vp_una2moe](U)[ps].[fl2r] | 13 | [5am6_p_m](A)[ps].m(C) | 52 |
| | siSSB | (U)[ps].m(A)p.m(C)p.m(A) | | [ps].[rc16](A)p.m(A)p.m(C)p.m(A) | |
| | | p.[fl2r](U)p.m(U)p.m(A)p.m(A) | | p.[fl2r](G)p.[fl2r](A)p.[fl2r](C) | |
| | | p.m(A)p.m(G)p.m(U)p.m(C) | | p.m(U)p.m(U)p.m(U)p.m(A)p.m | |
| | | p.[fl2r](U)p.m(G)p.[fl2r](U)p.m | | (A)p.m(U)p.m(G)p.m(U)[ps].m | |
| | | (U)p.m(G)p.m(U)[ps].m(U)[ps].m | | (A)[ps].m(A) | |
| | | (U) | | | |
| 4 | pos17.C16. | [5vp_una2moe](U)[ps].[fl2r] | 13 | [5am6_p_m](A)[ps].m(C)[ps].m | 53 |
| | siSSB | (U)[ps].m(A)p.m(C)p.m(A) | | (A)p.m(A)p.m(C)p.m(A)p.[fl2r] | |
| | | p.[fl2r](U)p.m(U)p.m(A)p.m(A) | | (G)p.[fl2r](A)p.[fl2r](C)p.m | |
| | | p.m(A)p.m(G)p.m(U)p.m(C) | | (U)p.m(U)p.m(U)p.m(A)p.m(A) | |
| | | p.[fl2r](U)p.m(G)p.[fl2r](U)p.m | | p.m(U)p.m(G)p.[rc16](U)[ps].m | |
| | | (U)p.m(G)p.m(U)[ps].m(U)[ps].m | | (A)[ps].m(A) | |
| | | (U) | | | |
| 5 | pos19.C16. | [5vp_una2moe](U)[ps].[fl2r] | 13 | [5am6_p_m](A)[ps].m(C)[ps].m | 54 |
| | siSSB | (U)[ps].m(A)p.m(C)p.m(A) | | (A)p.m(A)p.m(C)p.m(A)p.[fl2r] | |
| | | p.[fl2r](U)p.m(U)p.m(A)p.m(A) | | (G)p.[fl2r](A)p.[fl2r](C)p.m | |
| | | p.m(A)p.m(G)p.m(U)p.m(C) | | (U)p.m(U)p.m(U)p.m(A)p.m(A) | |
| | | p.[fl2r](U)p.m(G)p.[fl2r](U)p.m | | p.m(U)p.m(G)p.m(U)[ps].m(A) | |
| | | (U)p.m(G)p.m(U)[ps].m(U)[ps].m | | [ps].[rc16](A) | |
| | | (U) | | | |

5vp_una2moe 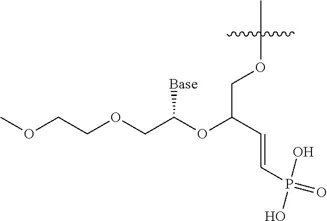
5am6_p_m 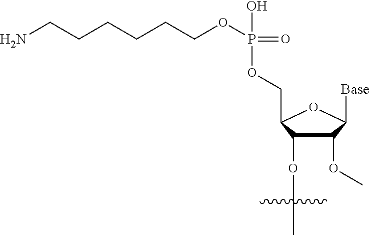
rc16

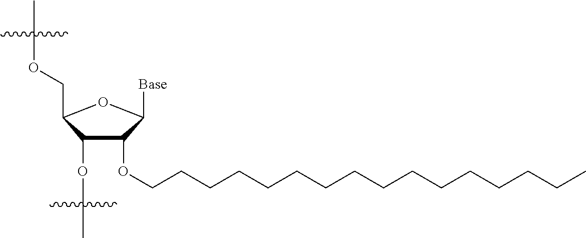

In Table 12, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

TABLE 13
K_{d} of mAb-siRNA DAR1 conjugates to HSA as
detected by fluorescence polarimetry assay.

| Sample | K_{d} (μM) |
|---|---|
| mTfR1-mAb-siSSB DAR1 | 102 |
| mTfR1-mAb-pos6.C16.siSSB DAR1 | 2.38 |
| mTfR1-mAb-pos3.C16.siSSB DAR1 | 41.9 |
| mTfR1-mAb-pos17.C16.siSSB DAR1 | 2.36 |
| mTfR1-mAb-pos19.C16.siSSB DAR1 | 1.04 |

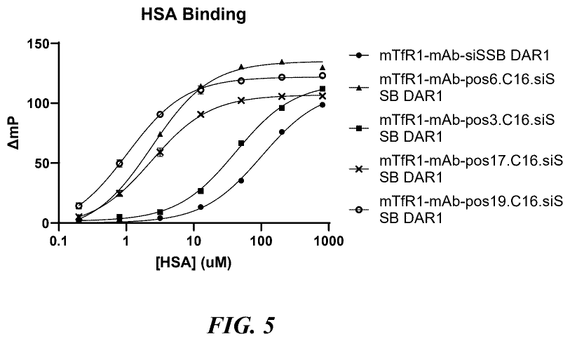

As shown in Table 13, the fluorescent HSA binding assay showed that the HSA binding of the lipid AOC with the C16 lipid attached to the passenger strand at position 3 (mTfR1-mAb.pos3.C16.siSSB) had a K_{d} of 41 μM represent over a 2-fold K_{d} enhancement over the control AOC (mTfR1-mAb.siSSB). As shown in FIG. 5, the lipid AOC (mTfR1-mAb.pos3.C16.siSSB) showed more than 50 to 100 fold enhancement in EC50 binding to HSA compared to the lipid AOC with the C16 hydrocarbon chain (C16 lipid) attached to position 6 (mTfR1-mAb.pos.C16.siSSB), position 17 (mTfR1-mAb.pos17.C16.siSSB), or position 19 (mTfR1-mAb.pos19.C16.siSSB) of the passenger strand.

SSB siRNA tissue concentrations and SSB mRNA expression levels were assessed with DAR1 lipid AOC with the C16 hydrocarbon chain (C16 lipid) attached at position 3 (mTfR1-mAb.pos3.C16.siSSB), position 6 (mTfR1-mAb.pos6.C16.siSSB), position 17 (mTfR1-mAb.pos17.C16.siSSB), or position 19 (mTfR1-mAb.pos6.C19.siSSB) of the passenger strand at day 28 and day 56 in the gastroc, tibialis anterior (TA), liver, or heart tissue obtained from mice following a single injection of a DAR1 control AOC (mTfR1-mAb-siSSB) or the DAR1 lipid AOC with C16 hydrocarbon chain (C16 lipid) attached at position 3, 6, 17 or 19 of the passenger strand of the siRNA at Day 0.

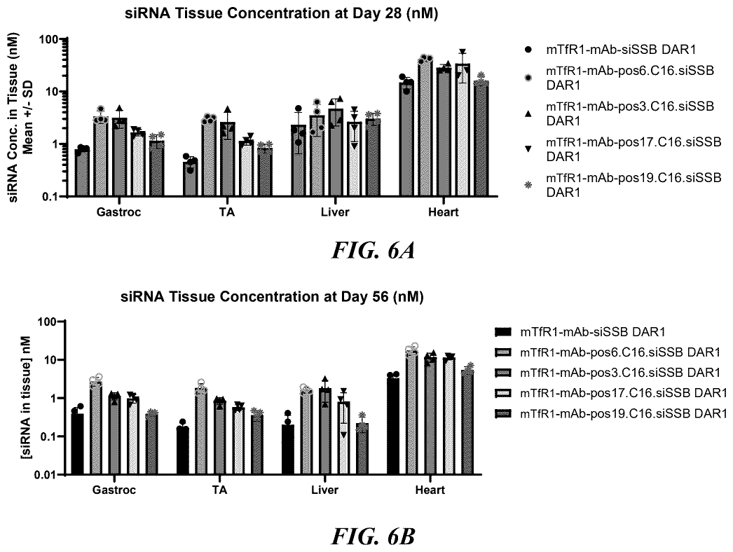

At day 28, the lipid AOC with C16 hydrocarbon chain attached position 3 or position 6 delivered more siRNA to tissues than the control AOC in the gastroc and TA, which are both skeletal muscles (FIG. 6A). In addition, these 2 lipid AOCs were delivered more siRNA to the tissues, resulting in higher concentration at the tissues than the 2 other AOCs in the gastroc, TA, and liver. Interestingly, the lipid AOC with C16 hydrocarbon chain attached at position 3 or position 6 deliver 2 to 3-fold more siRNA that the other 2 AOCs in skeletal muscles. However, all lipid AOCs deliver similar levels of siRNAs in the liver and heart.

At day 56, the lipid AOC with C16 hydrocarbon chain attached position 6 delivered more siRNA to tissues than the other lipid AOCs or control AOC in the gastroc, TA, and heart tissues (FIG. 6B). The lipid AOC at position 3 and 17 had similar activities in all 3 muscle tissues: gastroc, TA, and heart. However, there was no significant differences in siRNA tissue concentrations between the lipid AOC with C16 attached to position 19 and the control AOC in gastroc or liver.

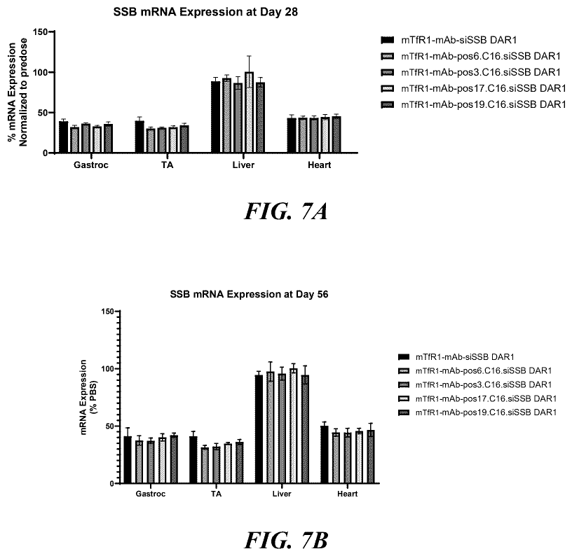

As shown in FIG. 7A, all lipid AOCs decrease mRNA expression levels in gastroc and TA compared to the control AOC, which are both skeletal muscles at Day 28. However, All lipid AOCs had no differences in mRNA expression levels compared to control AOC in the liver and heart. In addition, all lipid AOCs decrease SSB mRNA expression levels in TA. The lipid AOCs with position 6 had the best activity in the TA muscle at day 56 (FIG. 7B). However, all lipid AOCs had no differences in mRNA expression levels compared to control AOC in the gastroc, liver and heart.

Overall, lipid AOCs the lipid AOC with C16 hydrocarbon chain conjugated to position 3 or position 6 of passenger strand of siRNA delivered the highest concentrations of siRNA in skeletal muscle tissues and provided long lasting reduction in mRNA expression levels in muscle tissues.

### Example 7. Lipid Length Optimization at Passenger Strand Position 6 on mTfR1-mAb-siSSB DAR1 AOCs in CD1 Mice

SSB: A 21-mer double-stranded siRNA with 19 bases of complementarity and 3′ dinucleotide overhangs was designed against mouse SSB. The sequence (5′ to 3′) of the guide/antisense strand was UUACAUUAAAGUCUGUUGUUU (SEQ ID NO: 5). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double stranded siRNA. The passenger strand of the siRNA contained a conjugation handle, a C6-NH2, at the 5′ end, which was connected to the siRNA passenger strand via phosphorothioate-inverted abasic-phosphorothioate linker.

### Conjugate Synthesis and Characterization

The CD71 mAb-siRNA DAR1 conjugates were made, purified and characterized as described in Example 1-3. All conjugates were made through cysteine conjugation using a bismal linker. Conjugates were characterized chromatographically as described in Table 14.

### Table 14: HPLC Retention Time (RT) in Minutes

TABLE 14
HPLC Retention Time (RT) in Minutes

| | | RT, SAX | RT, SEC |
|---|---|---|---|
| Group(s) | Conjugate | Method-3 | Method-1 |
| 1 | mTfR1-mAb-siSSB DAR1 | 3.735 | 3.021 |
| 2 | mTfR1-mAb-pos6.tzC14.siSSB DAR1 | 3.755 | 3.171 |
| 3 | mTfR1-mAb-pos6.tzC16.siSSB DAR1 | 3.764 | 3.087 |
| 4 | mTfR1-mAb-pos6.tzC18.siSSB DAR1 | 3.779 | 3.114 |
| 5 | mTfR1-mAb-pos6.tzC20.siSSB DAR1 | 3.796 | 3.182 |
| 6 | mTfR1-mAb-pos6.tzC18-oleyl.siSSB DAR1 | 3.731 | 3.001 |
| 7 | mTfR1-mAb-pos6.tzC18-int.acid.siSSB DAR1 | 3.737 | 3.016 |
| 8 | mTfR1-mAb-pos6.tzC20da.siSSB DAR1 | 3.729 | 3.066 |
| 9 | mTfR1-mAb-pos.3.C16.pos6.C16.siSSB DAR1 | 3.737 | 3.144 |
| 10 | mTfR1-mAb-pos6.C16.siSSB DAR1 | 3.698 | 3.014 |

### In Vivo Study Design

The conjugates were assessed for their ability to mediate mRNA downregulation of SSB in skeletal muscle in vivo in wild type CD-1 mice. Mice were dosed via intravenous (IV) injection with PBS vehicle control or the indicated ASCs and at 0.5 mg/kg siRNA dose as shown in Table 15. After 28 days, gastrocnemius (gastroc), tibialis anterior (TA), liver, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 15
In vivo study design testing duration of action of SSB siRNA mAb DAR1 AOCs containing
2′ lipid modifications at passenger strand position 3 or 6 in CD1 Mice

| | | | siRNA:mAb | siRNA | | |
|---|---|---|---|---|---|---|
| | | | Ratio | Dose | Doses | Harvest |
| Group | Test Article | N | (mol/mol) | (mg/kg) | (#) | (day) |
| 1 | mTfR1-mAb-siSSB DAR1 | 4 | 1 | 0.5 | 1 | 28 |
| 2 | mTfR1-mAb-pos6.tzC14.siSSB | 4 | 1 | 0.5 | 1 | 28 |
| | DAR1 | | | | | |
| 3 | mTfR1-mAb-pos6.tzC16.siSSB | 4 | 1 | 0.5 | 1 | 28 |
| | DAR1 | | | | | |
| 4 | mTfR1-mAb-pos6.tzC18.siSSB | 4 | 1 | 0.5 | 1 | 28 |
| | DAR1 | | | | | |
| 5 | mTfR1-mAb-pos6.tzC20.siSSB | 4 | 1 | 0.5 | 1 | 28 |
| | DAR1 | | | | | |
| 6 | mTfR1-mAb-pos6.tzC18- | 4 | 1 | 0.5 | 1 | 28 |
| | oleyl.siSSB DAR1 | | | | | |
| 7 | mTfR1-mAb-pos6.tzC18- | 4 | 1 | 0.5 | 1 | 28 |
| | int.acid.siSSB DAR1 | | | | | |
| 8 | mTfR1-mAb-pos6.tzC20da.siSSB | 4 | 1 | 0.5 | 1 | 28 |
| | DAR1 | | | | | |
| 9 | mTfR1-mAb- | 4 | 1 | 0.5 | 1 | 28 |
| | pos.3.C16.pos6.C16.siSSB DAR1 | | | | | |
| 10 | mTfR1-mAb-pos6.C16.siSSB | 4 | 1 | 0.5 | 1 | 28 |
| | DAR1 | | | | | |
| 11 | PBS | 4 | N/A | N/A | 1 | 28 |

TABLE 16
siRNA Sequences used in Example 7

| | | | | | SEQ |
|---|---|---|---|---|---|
| Compound | | | SEQ | | ID |
| No. | siRNA Ref. | siRNA Guide Sequence | ID NO: | siRNA Passenger Sequence | NO: |
| 1 | siSSB | [5vp_una2moe](U)[ps].[fl2r] | 13 | [5am6_p_m](A)[ps].m(C) | 50 |
| | | (U)[ps].m(A)p.m(C)p.m | | [ps].m(A)p.m(A)p.m(C)p.m | |
| | | (A)p.[fl2r](U)p.m(U)p.m(A) | | (A)p.[fl2r](G)p.[fl2r](A)p.[fl2r] | |
| | | p.m(A)p.m(A)p.m(G)p.m(U) | | (C)p.m(U)p.m(U)p.m(U)p.m | |
| | | p.m(C)p.[fl2r](U)p.m(G)p.[fl2r] | | (A)p.m(A)p.m(U)p.m(G)p.m | |
| | | (U)p.m(U)p.m(G)p.m(U) | | (U)[ps].m(A)[ps].m(A) | |
| | | [ps].m(U)[ps].m(U) | | | |
| 2 | pos6.tzC14. | [5vp_una2moe](U)[ps].[fl2r] | 13 | [5am6_p_m](A)[ps].m(C) | 55 |
| | siSSB | (U)[ps].m(A)p.m(C)p.m | | [ps].m(A)p.m(A)p.m(C)p.[tzc14] | |
| | | (A)p.[fl2r](U)p.m(U)p.m(A) | | (A)p.[fl2r](G)p.[fl2r](A) | |
| | | p.m(A)p.m(A)p.m(G)p.m(U) | | p.[fl2r](C)p.m(U)p.m(U)p.m | |
| | | p.m(C)p.[fl2r](U)p.m(G)p.[fl2r] | | (U)p.m(A)p.m(A)p.m(U)p.m | |
| | | (U)p.m(U)p.m(G)p.m(U) | | (G)p.m(U)[ps].m(A)[ps].m(A) | |
| | | [ps].m(U)[ps].m(U) | | | |
| 3 | pos6.tzC16. | [5vp_una2moe](U)[ps].[fl2r] | 13 | [5am6_p_m](A)[ps].m(C) | 56 |
| | siSSB | (U)[ps].m(A)p.m(C)p.m | | [ps].m(A)p.m(A)p.m(C)p.[tzc16] | |
| | | (A)p.[fl2r](U)p.m(U)p.m(A) | | (A)p.[fl2r](G)p.[fl2r](A) | |
| | | p.m(A)p.m(A)p.m(G)p.m(U) | | p.[fl2r](C)p.m(U)p.m(U)p.m | |
| | | p.m(C)p.[fl2r](U)p.m(G)p.[fl2r] | | (U)p.m(A)p.m(A)p.m(U)p.m | |
| | | (U)p.m(U)p.m(G)p.m(U) | | (G)p.m(U)[ps].m(A)[ps].m(A) | |
| | | [ps].m(U)[ps].m(U) | | | |
| 4 | pos6.tzC18. | [5vp_una2moe](U)[ps].[fl2r] | 13 | [5am6_p_m](A)[ps].m(C) | 57 |
| | siSSB | (U)[ps].m(A)p.m(C)p.m | | [ps].m(A)p.m(A)p.m(C)p.[tzc18] | |
| | | (A)p.[fl2r](U)p.m(U)p.m(A) | | (A)p.[fl2r](G)p.[fl2r](A) | |
| | | p.m(A)p.m(A)p.m(G)p.m(U) | | p.[fl2r](C)p.m(U)p.m(U)p.m | |
| | | p.m(C)p.[fl2r](U)p.m(G)p.[fl2r] | | (U)p.m(A)p.m(A)p.m(U)p.m | |
| | | (U)p.m(U)p.m(G)p.m(U) | | (G)p.m(U)[ps].m(A)[ps].m(A) | |
| | | [ps].m(U)[ps].m(U) | | | |
| 5 | pos6.tzC20. | [5vp_una2moe](U)[ps].[fl2r] | 13 | [5am6_p_m](A)[ps].m(C) | 58 |
| | siSSB | (U)[ps].m(A)p.m(C)p.m | | [ps].m(A)p.m(A)p.m(C)p.[tzc20] | |
| | | (A)p.[fl2r](U)p.m(U)p.m(A) | | (A)p.[fl2r](G)p.[fl2r](A) | |
| | | p.m(A)p.m(A)p.m(G)p.m(U) | | p.[fl2r](C)p.m(U)p.m(U)p.m | |
| | | p.m(C)p.[fl2r](U)p.m(G)p.[fl2r] | | (U)p.m(A)p.m(A)p.m(U)p.m | |
| | | (U)p.m(U)p.m(G)p.m(U) | | (G)p.m(U)[ps].m(A)[ps].m(A) | |
| | | [ps].m(U)[ps].m(U) | | | |
| 6 | pos6.tzC18- | [5vp_una2moe](U)[ps].[fl2r] | 13 | [5am6_p_m](A)[ps].m(C) | 59 |
| | oleyl.siSSB | (U)[ps].m(A)p.m(C)p.m | | [ps].m(A)p.m(A)p.m(C)p.[tzc18o] | |
| | | (A)p.[fl2r](U)p.m(U)p.m(A) | | (A)p.[fl2r](G)p.[fl2r](A) | |
| | | p.m(A)p.m(A)p.m(G)p.m(U) | | p.[fl2r](C)p.m(U)p.m(U)p.m | |
| | | p.m(C)p.[fl2r](U)p.m(G)p.[fl2r] | | (U)p.m(A)p.m(A)p.m(U)p.m | |
| | | (U)p.m(U)p.m(G)p.m(U) | | (G)p.m(U)[ps].m(A)[ps].m | |
| | | [ps].m(U)[ps].m(U) | | (A) | |
| 7 | pos6.tzC18- | [5vp_una2moe](U)[ps].[fl2r] | 13 | [5am6_p_m](A)[ps].m(C) | 60 |
| | int.acid. | (U)[ps].m(A)p.m(C)p.m | | [ps].m(A)p.m(A)p.m(C)p.[tzl18] | |
| | siSSB | (A)p.[fl2r](U)p.m(U)p.m(A) | | (A)p.[fl2r](G)p.[fl2r](A) | |
| | | p.m(A)p.m(A)p.m(G)p.m(U) | | p.[fl2r](C)p.m(U)p.m(U)p.m | |
| | | p.m(C)p.[fl2r](U)p.m(G)p.[fl2r] | | (U)p.m(A)p.m(A)p.m(U)p.m | |
| | | (U)p.m(U)p.m(G)p.m(U) | | (G)p.m(U)[ps].m(A)[ps].m(A) | |
| | | [ps].m(U)[ps].m(U) | | | |
| 8 | pos6. | [5vp_una2moe](U)[ps].[fl2r] | 13 | [5am6_p_m](A)[ps].m(C) | 61 |
| | tzC20da. | (U)[ps].m(A)p.m(C)p.m | | [ps].m(A)p.m(A)p.m(C)p. | |
| | siSSB | (A)p.[fl2r](U)p.m(U)p.m(A) | | [tzc20ada](A)p.[fl2r](G)p.[fl2r] | |
| | | p.m(A)p.m(A)p.m(G)p.m(U) | | (A)p.[fl2r](C)p.m(U)p.m(U) | |
| | | p.m(C)p.[fl2r](U)p.m(G)p.[fl2r] | | p.m(U)p.m(A)p.m(A)p.m(U) | |
| | | (U)p.m(U)p.m(G)p.m(U) | | p.m(G)p.m(U)[ps].m(A)[ps].m | |
| | | [ps].m(U)[ps].m(U) | | (A) | |
| 9 | pos.3.C16. | [5vp_una2moe](U)[ps].[fl2r] | 13 | [5am6_p_m](A)[ps].m(C) | 62 |
| | pos6.C16. | (U)[ps].m(A)p.m(C)p.m | | [ps].[rc16](A)p.m(A)p.m(C) | |
| | siSSB | (A)p.[fl2r](U)p.m(U)p.m(A) | | p.[rc16](A)p.[fl2r](G)p.[fl2r] | |
| | | p.m(A)p.m(A)p.m(G)p.m(U) | | (A)p.[fl2r](C)p.m(U)p.m(U) | |
| | | p.m(C)p.[fl2r](U)p.m(G)p.[fl2r] | | p.m(U)p.m(A)p.m(A)p.m(U) | |
| | | (U)p.m(U)p.m(G)p.m(U) | | p.m(G)p.m(U)[ps].m(A)[ps].m | |
| | | [ps].m(U)[ps].m(U) | | (A) | |
| 10 | pos6.C16. | [5vp_una2moe](U)[ps].[fl2r] | 13 | [5am6_p_m](A)[ps].m(C) | 51 |
| | siSSB | (U)[ps].m(A)p.m(C)p.m | | [ps].m(A)p.m(A)p.m(C)p.[rc16] | |
| | | (A)p.[fl2r](U)p.m(U)p.m(A) | | (A)p.[fl2r](G)p.[fl2r](A)p. | |
| | | p.m(A)p.m(A)p.m(G)p.m(U) | | [fl2r](C)p.m(U)p.m(U)p.m(U) | |
| | | p.m(C)p.[fl2r](U)p.m(G)p.[fl2r] | | p.m(A)p.m(A)p.m(U)p.m(G) | |
| | | (U)p.m(U)p.m(G)p.m(U) | | p.m(U)[ps].m(A)[ps].m(A) | |
| | | [ps].m(U)[ps].m(U) | | | |

5vp_una2moe 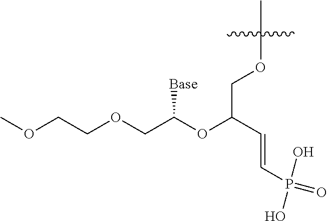
5am6_p_m 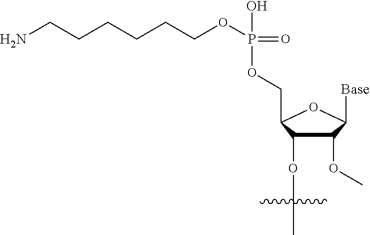
tzc14 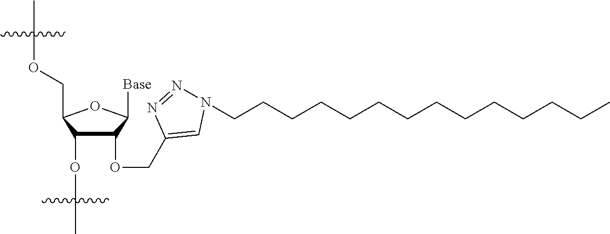
tzc16 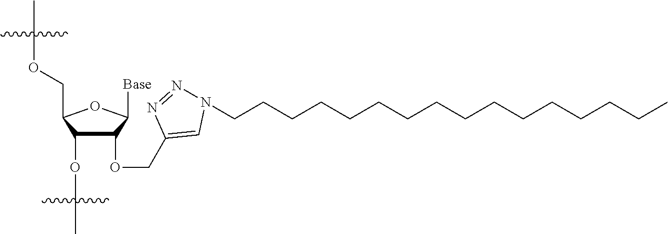
tzc18 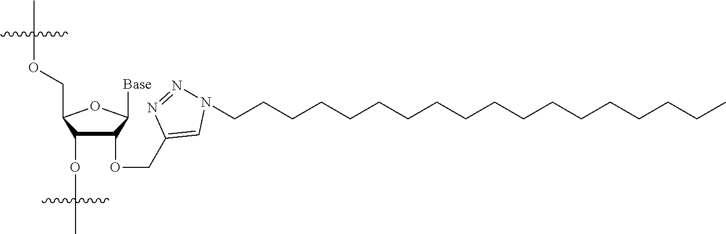
tzc20 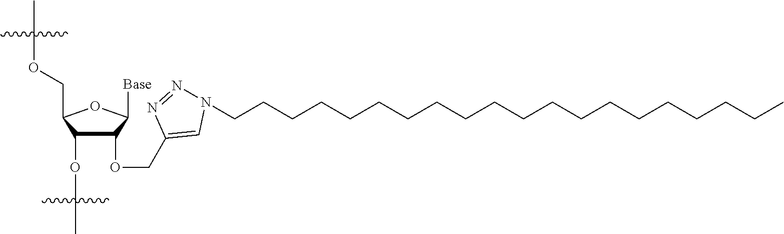
tzc18o 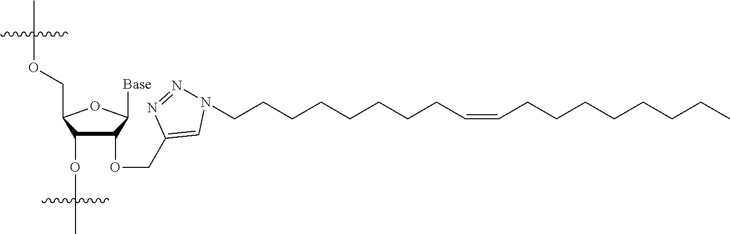
tz118 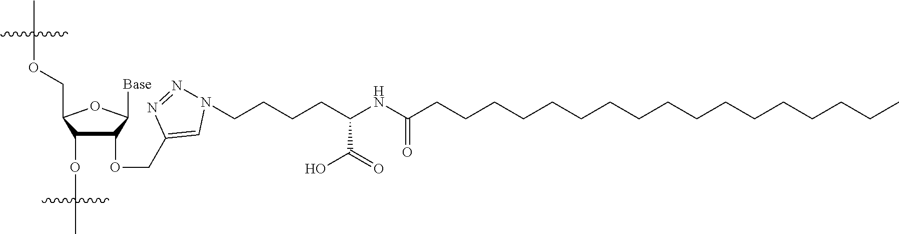
tzada20 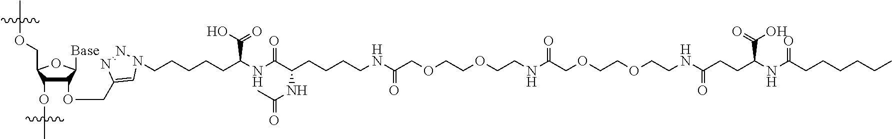
rc16

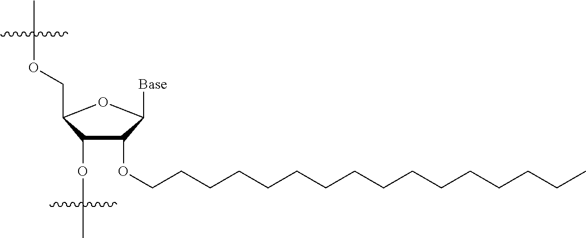

In Table 16, m indicates the 2-O-methyl (2-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

### Results

A copper-mediated click-chemistry structure activity relationship approach was carried out to optimize the lipid carbon chain length for the transferrin-receptor-mediated targeted delivery of siRNA to muscle cells. In particular, the siRNA SSB passenger strand was modified at position 6 with 2′-O-propargyl and copper catalyzed click chemistry was performed various azide-modified lipids with lengths ranging from C14 to C20 hydrocarbons in two-carbon increments. Additional lipid classes were also explored by including oleyl and carboxylic acids. The resultant triazole (“tz”) modified lipids were found to be comparable in length to the n+2 the length of 2′-O modified lipid. For example, the tzC12 is approximately equal to a 2′-O—C14.

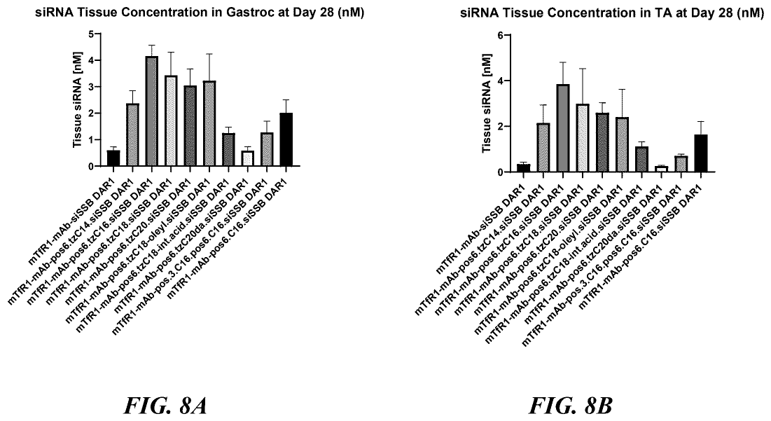

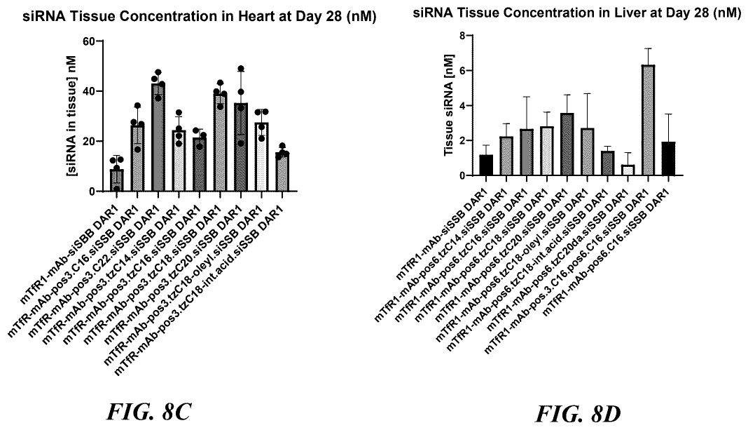

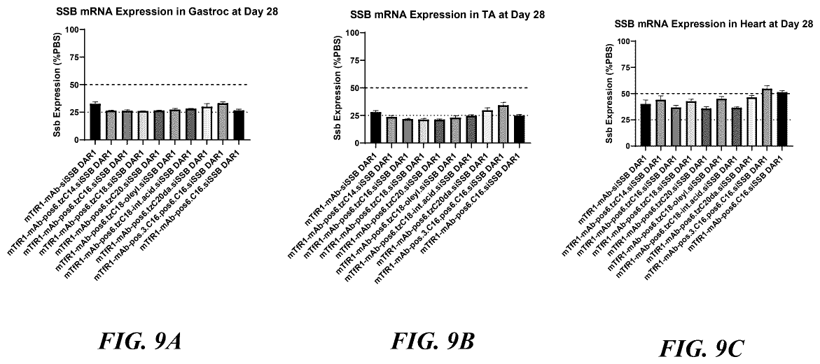

As shown in FIGS. 8A-D and FIGS. 9A-C, SSB siRNA tissue concentrations and SSB mRNA expression levels were assessed with DAR1 lipid AOC with numerous triazole (tz) modified lipids having different lengths (TzC14, TzC16, TzC18, TzC20, TzC18-oleyl, TzC18 int.acid, TzC20da) attached at position 6 of the passenger strand at day 28 in the gastroc, tibialis anterior (TA), heart, or liver tissue obtained from mice following a single injection of a DAR1 control AOC (mTfR1-mAb-siSSB) or the DAR1 lipid-AOC with the numerous different lipids attached at position 3 or position 6 of the passenger strand (mTfR1-mAb-pos6.tzC14.siSSB, mTfR1-mAb-pos6.tzC16.siSSB, mTfR1-mAb-pos6.tzC18.siSSB, mTfR1-mAb-pos6.tzC20.siSSB, mTfR1-mAb-pos6.tzC18-oleyl.siSSB, mTfR1-mAb-pos6.tzC18-int.acid.siSSB, mTfR1-mAb-pos6.tzC20da.siSSB, mTfR1-mAb-pos.3.C16.pos6.C16.siSSB, mTfR1-mAb-pos6.C16.siSSB) of the siRNA at Day 0.

Most triazole (tz) modified lipid AOCs with the lipids in different lengths were able to deliver higher siRNA to all 4 tissues compared to the control AOCs at day 28 (FIGS. 8A-D). Among the tz modified lipid AOCs, the tz modified lipid AOCs with the modified lipid TzC16 (mTfR1-mAb-pos6.tzC16.siSSB) showed the best activities in gastroc and TA tissues by delivering a 7-fold higher amount of siRNAs in gastroc and more than 10-fold higher amount of siRNAs in the TA (FIGS. 8A-B). In the liver, the lipid AOCs with the C16 hydrocarbon chain conjugated at position 3 showed the best activities (FIG. 8D). Interestingly, the tz modified lipid AOCs with the modified lipid tzC20da was not able to deliver increased amounts of siRNA to tissues compared to control AOCs.

At Day 28, no significant differences could be observed in the reduction in mRNA expression levels for the lipid-AOCs compared to control AOC (FIGS. 9A-C). All tz modified lipid AOCs and control AOCs as shown in FIGS. 9A-C could reduce SSB mRNA expression levels to 25% in gastroc and TA, and up to 50% in the heart.

Overall, several tz modified lipid including tzC16, tzC18, tzC20, tzC18-oleyl and lipid 2′-O—C16 were able to improve the delivery of lipid-modified siRNA to muscle tissue and when compared to the non-lipid modified control. The optimal lipid length at passenger strand position 6 to deliver the most siRNA to the target tissue was the tz modified lipid tzC16 corresponding to a 2′-O—C18. Triazole modified lipid AOCs were able to deliver higher amounts of siRNA in muscle cells, but they did not enhance the reduction in mRNA expression levels in these tissues.

### Example 8: Lipid Length Optimization at Passenger Strand Position 3 on mTfR1-mAb-siSSB DAR1 AOCs in CD1 Mice

SSB: A 21-mer double-stranded siRNA with 19 bases of complementarity and 3′ dinucleotide overhangs was designed against mouse SSB. The sequence (5′ to 3′) of the guide/antisense strand was UUACAUUAAAGUCUGUUGUUU (SEQ ID NO: 5). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double stranded siRNA. The passenger strand contained a conjugation handle, a C6-NH2, at the 5′ end, which was connected to the siRNA passenger strand via phosphorothioate-inverted abasic-phosphorothioate linker.

### Conjugate Synthesis and Characterization

The CD71 mAb-siRNA DAR1 conjugates were made, purified and characterized as described in Examples 1-3. All conjugates were made through cysteine conjugation using a bismal linker. Conjugates were characterized chromatographically as described in Table 17.

TABLE 17
HPLC Retention Time (RT) in Minutes

| | | RT, SAX | RT, SEC |
|---|---|---|---|
| Group | Conjugate | Method-3 | Method-1 |
| 1 | mTfR1-mAb-siSSB DAR1 | 3.528 | 3.001 |
| 2 | mTfR1-mAb-pos3.C16.siSSB DAR1 | 3.504 | 3.018 |
| 3 | mTfR1-mAb-pos3.C22.siSSB DAR1 | 3.527 | 3.051 |
| 4 | mTfR1-mAb-pos3.tzC14.siSSB DAR1 | 3.512 | 3.022 |
| 5 | mTfR1-mAb-pos3.tzC16.siSSB DAR1 | 3.505 | 3.030 |
| 6 | mTfR1-mAb-pos3.tzC18.siSSB DAR1 | 3.503 | 3.032 |
| 7 | mTfR1-mAb-pos3.tzC20.siSSB DAR1 | 3.507 | 3.052 |
| 8 | mTfR1-mAb-pos3.tzC18-oleyl.siSSB | 3.492 | 3.041 |
| | DAR1 | | |
| 9 | mTfR1-mAb-pos3.tzC18-int.acid.siSSB | 3.512 | 3.016 |
| | DAR1 | | |

### In Vivo Study Design

The conjugates were assessed for their ability to mediate mRNA downregulation of SSB in skeletal muscle in vivo in wild type CD-1 mice. Mice were dosed via intravenous (IV) injection with PBS vehicle control or the indicated ASCs and at 0.5 mg/kg siRNA dose as shown in Table 18. After 28 days, gastrocnemius (gastroc), tibialis anterior (TA), liver, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 18
In vivo study design testing duration of action of SSB
siRNA mAb DAR1 AOCs containing 2′ lipid modifications
at passenger strand position 3 or 6 in CD1 Mice

| | | | siRNA:mAb | siRNA | | |
|---|---|---|---|---|---|---|
| | | | Ratio | Dose | Doses | Harvest |
| Group(s) | Test Article | N | (mol/mol) | (mg/kg) | (#) | (day) |
| 1 | mTfR1-mAb-siSSB DAR1 | 4 | 1 | 0.5 | 1 | 28 |
| 2 | mTfR1-mAb- | 4 | 1 | 0.5 | 1 | 28 |
| | pos3.C16.siSSB DAR1 | | | | | |
| 3 | mTfR1-mAb- | 4 | 1 | 0.5 | 1 | 28 |
| | pos3.C22.siSSB DAR1 | | | | | |
| 4 | mTfR1-mAb- | 4 | 1 | 0.5 | 1 | 28 |
| | pos3.tzC14.siSSB DAR1 | | | | | |
| 5 | m TfR1-mAb- | 4 | 1 | 0.5 | 1 | 28 |
| | pos3.tzC16.siSSB DAR1 | | | | | |
| 6 | mTfR1-mAb- | 4 | 1 | 0.5 | 1 | 28 |
| | pos3.tzC18.siSSB DAR1 | | | | | |
| 7 | mTfR1-mAb- | 4 | 1 | 0.5 | 1 | 28 |
| | pos3.tzC20.siSSB DAR1 | | | | | |
| 8 | mTfR1-mAb-pos3.tzC18- | 4 | 1 | 0.5 | 1 | 28 |
| | oleyl.siSSB DAR1 | | | | | |
| 9 | mTfR1-mAb-pos3.tzC18- | 4 | 1 | 0.5 | 1 | 28 |
| | int.acid.siSSB DAR1 | | | | | |
| 10 | PBS | 4 | — | — | — | 28 |

TABLE 19
siRNA Sequences used in Example 8

| | | | SEQ | | SEQ |
|---|---|---|---|---|---|
| Compound | SIRNA | | ID | | ID |
| No. | Ref. | siRNA Guide Sequence | NO: | siRNA Passenger Sequence | NO: |
| 1 | siSSB | [5vp_una2moe](U)[ps].[fl2r] | 13 | [5am6_p_m](A)[ps].m(C) | 50 |
| | | (U)[ps].m(A)p.m(C)p.m | | [ps].m(A)p.m(A)p.m(C)p.m(A)p. | |
| | | (A)p.[fl2r](U)p.m(U)p.m(A) | | [fl2r](G)p.[fl2r](A)p.[fl2r](C)p.m | |
| | | p.m(A)p.m(A)p.m(G)p.m(U) | | (U)p.m(U)p.m(U)p.m(A)p.m(A) | |
| | | p.m(C)p.[fl2r](U)p.m(G) | | p.m(U)p.m(G)p.m(U)[ps].m(A) | |
| | | p.[fl2r](U)p.m(U)p.m(G)p.m | | [ps].m(A) | |
| | | (U)[ps].m(U)[ps].m(U) | | | |
| 2 | pos3.C16. | [5vp_una2moe](U)[ps].[fl2r] | 13 | [5am6_p_m](A)[ps].m(C) | 52 |
| | siSSB | (U)[ps].m(A)p.m(C)p.m | | [ps].[rc16](A)p.m(A)p.m(C)p.m | |
| | | (A)p.[fl2r](U)p.m(U)p.m(A) | | (A)p.[fl2r](G)p.[fl2r](A)p.[fl2r] | |
| | | p.m(A)p.m(A)p.m(G)p.m(U) | | (C)p.m(U)p.m(U)p.m(U)p.m(A) | |
| | | p.m(C)p.[fl2r](U)p.m(G) | | p.m(A)p.m(U)p.m(G)p.m(U)[ps].m | |
| | | p.[fl2r](U)p.m(U)p.m(G)p.m | | (A)[ps].m(A) | |
| | | (U)[ps].m(U)[ps].m(U) | | | |
| 3 | pos3.C22. | [5vp_una2moe](U)[ps].[fl2r] | 13 | [5am6_p_m](A)[ps].m(C) | 63 |
| | siSSB | (U)[ps].m(A)p.m(C)p.m | | [ps].[rc22](A)p.m(A)p.m(C)p.m | |
| | | (A)p.[fl2r](U)p.m(U)p.m(A) | | (A)p.[fl2r](G)p.[fl2r](A)p.[fl2r] | |
| | | p.m(A)p.m(A)p.m(G)p.m(U) | | (C)p.m(U)p.m(U)p.m(U)p.m(A) | |
| | | p.m(C)p.[fl2r](U)p.m(G) | | p.m(A)p.m(U)p.m(G)p.m(U)[ps].m | |
| | | p.[fl2r](U)p.m(U)p.m(G)p.m | | (A)[ps].m(A) | |
| | | (U)[ps].m(U)[ps].m(U) | | | |
| 4 | pos3.tzC14. | [5vp_una2moe](U)[ps].[fl2r] | 13 | [5am6_p_m](A)[ps].m(C) | 64 |
| | siSSB | (U)[ps].m(A)p.m(C)p.m | | [ps].[tzc14](A)p.m(A)p.m(C)p.m | |
| | | (A)p.[fl2r](U)p.m(U)p.m(A) | | (A)p.[fl2r](G)p.[fl2r](A)p.[fl2r] | |
| | | p.m(A)p.m(A)p.m(G)p.m(U) | | (C)p.m(U)p.m(U)p.m(U)p.m(A) | |
| | | p.m(C)p.[fl2r](U)p.m(G) | | p.m(A)p.m(U)p.m(G)p.m(U) | |
| | | p.[fl2r](U)p.m(U)p.m(G)p.m | | [ps].m(A)[ps].m(A) | |
| | | (U)[ps].m(U)[ps].m(U) | | | |
| 5 | pos3.tzC16. | [5vp_una2moe](U)[ps].[fl2r] | 13 | [5am6_p_m](A)[ps].m(C) | 65 |
| | siSSB | (U)[ps].m(A)p.m(C)p.m | | [ps].[tzc16](A)p.m(A)p.m(C)p.m | |
| | | (A)p.[fl2r](U)p.m(U)p.m(A) | | (A)p.[fl2r](G)p.[fl2r](A)p.[fl2r] | |
| | | p.m(A)p.m(A)p.m(G)p.m(U) | | (C)p.m(U)p.m(U)p.m(U)p.m(A) | |
| | | p.m(C)p.[fl2r](U)p.m(G) | | p.m(A)p.m(U)p.m(G)p.m(U) | |
| | | p.[fl2r](U)p.m(U)p.m(G)p.m | | [ps].m(A)[ps].m(A) | |
| | | (U)[ps].m(U)[ps].m(U) | | | |
| 6 | pos3.tzC18. | [5vp_una2moe](U)[ps].[fl2r] | 13 | [5am6_p_m](A)[ps].m(C) | 66 |
| | siSSB | (U)[ps].m(A)p.m(C)p.m | | [ps].[tzc20](A)p.m(A)p.m(C)p.m | |
| | | (A)p.[fl2r](U)p.m(U)p.m(A) | | (A)p.[fl2r](G)p.[fl2r](A)p.[fl2r] | |
| | | p.m(A)p.m(A)p.m(G)p.m(U) | | (C)p.m(U)p.m(U)p.m(U)p.m(A) | |
| | | p.m(C)p.[fl2r](U)p.m(G) | | p.m(A)p.m(U)p.m(G)p.m(U) | |
| | | p.[fl2r](U)p.m(U)p.m(G)p.m | | [ps].m(A)[ps].m(A) | |
| | | (U)[ps].m(U)[ps].m(U) | | | |
| 7 | pos3.tzC20. | [5vp_una2moe](U)[ps].[fl2r] | 13 | [5am6_p_m](A)[ps].m(C) | 67 |
| | siSSB | (U)[ps].m(A)p.m(C)p.m | | [ps].[tzc18](A)p.m(A)p.m(C)p.m | |
| | | (A)p.[fl2r](U)p.m(U)p.m(A) | | (A)p.[fl2r](G)p.[fl2r](A)p.[fl2r] | |
| | | p.m(A)p.m(A)p.m(G)p.m(U) | | (C)p.m(U)p.m(U)p.m(U)p.m(A) | |
| | | p.m(C)p.[fl2r](U)p.m(G) | | p.m(A)p.m(U)p.m(G)p.m(U) | |
| | | p.[fl2r](U)p.m(U)p.m(G)p.m | | [ps].m(A)[ps].m(A) | |
| | | (U)[ps].m(U)[ps].m(U) | | | |
| 8 | pos3.tzC18- | [5vp_una2moe](U)[ps].[fl2r] | 13 | [5am6_p_m](A)[ps].m(C) | 68 |
| | oleyl.siSSB | (U)[ps].m(A)p.m(C)p.m | | [ps].[tzc180](A)p.m(A)p.m(C)p.m | |
| | | (A)p.[fl2r](U)p.m(U)p.m(A) | | (A)p.[fl2r](G)p.[fl2r](A)p.[fl2r] | |
| | | p.m(A)p.m(A)p.m(G)p.m(U) | | (C)p.m(U)p.m(U)p.m(U)p.m(A) | |
| | | p.m(C)p.[fl2r](U)p.m(G)p. | | p.m(A)p.m(U)p.m(G)p.m(U) | |
| | | [fl2r](U)p.m(U)p.m(G)p.m | | [ps].m(A)[ps].m(A) | |
| | | (U)[ps].m(U)[ps].m(U) | | | |
| 9 | pos3. | [5vp_una2moe](U)[ps].[fl2r] | 13 | [5am6_p_m](A)[ps].m(C) | 69 |
| | tzC18int. | (U)[ps].m(A)p.m(C)p.m | | [ps].[tzl18](A)p.m(A)p.m(C)p.m | |
| | acid. | (A)p.[fl2r](U)p.m(U)p.m(A) | | (A)p.[fl2r](G)p.[fl2r](A)p.[fl2r] | |
| | siSSB | p.m(A)p.m(A)p.m(G)p.m(U) | | (C)p.m(U)p.m(U)p.m(U)p.m(A) | |
| | | p.m(C)p.[fl2r](U)p.m(G) | | p.m(A)p.m(U)p.m(G)p.m(U) | |
| | | p.[fl2r](U)p.m(U)p.m(G)p.m | | [ps].m(A)[ps].m(A) | |
| | | (U)[ps].m(U)[ps].m(U) | | | |

5vp_una2moe 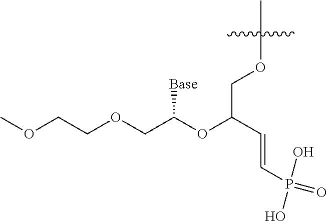
5am6_p_m 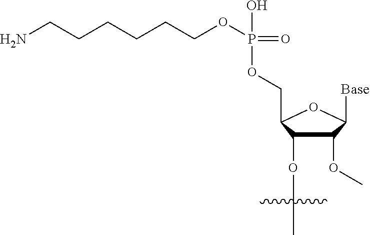
tzc14 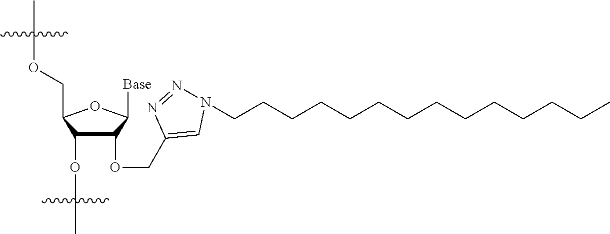
tzc16 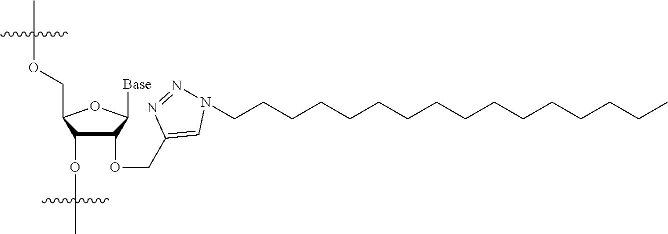
tzc18 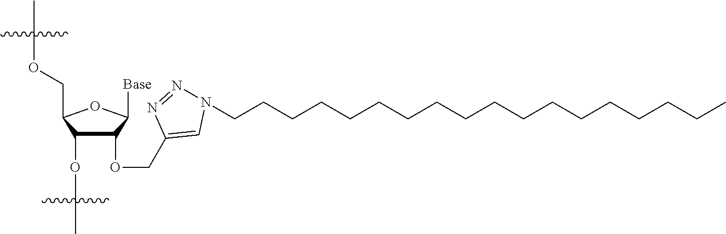
tzc20 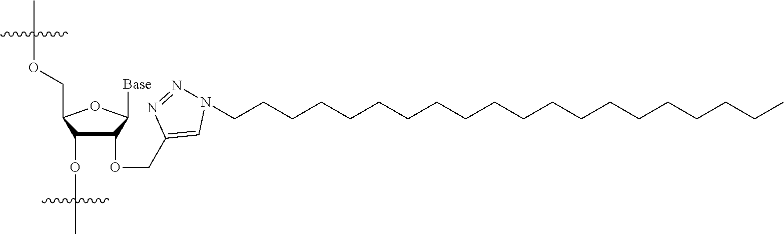
tzc18o 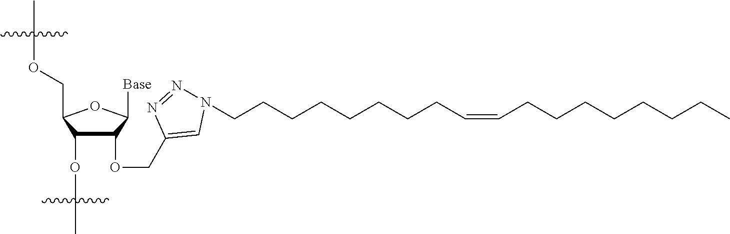
tz118 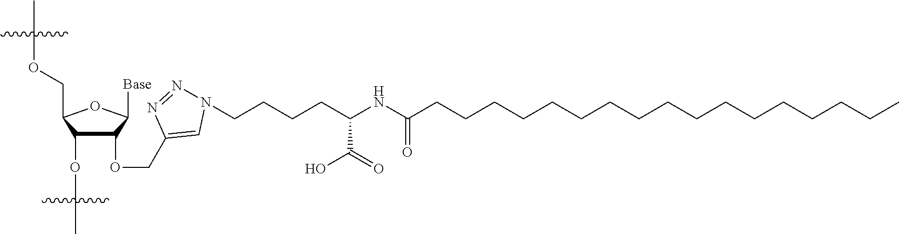
rc16 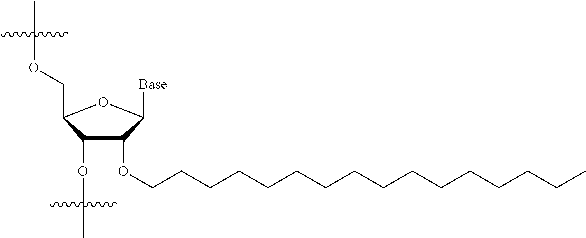
rc22

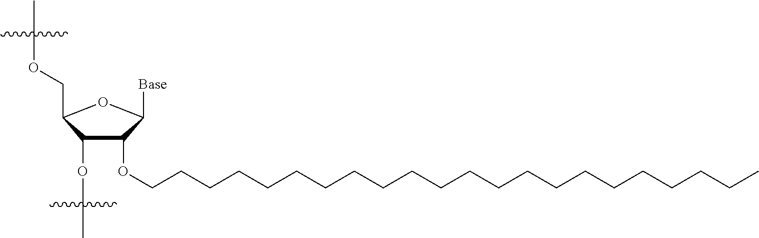

In Table 19, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

### Results

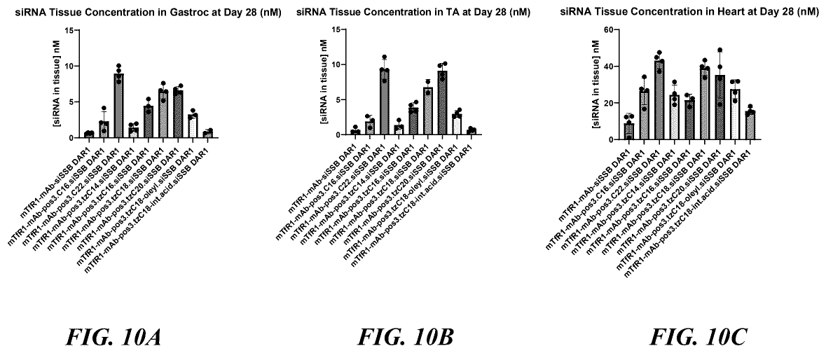

As shown in FIGS. 10A-C, SSB siRNA tissue concentrations and SSB mRNA expression levels were assessed with DAR1 lipid-AOC with numerous tz modified lipids having different lengths ranging from C14 to C20 as well as oleyl, and a carboxylic acid-modified lipid (C22, TzC14, TzC16, TzC18, TzC20, TzC18-oleyl, TzC18 int.acid) attached at position 3 of the passenger strand at day 28 in the gastroc, tibialis anterior (TA), and heart obtained from mice following a single injection of a DAR1 control AOC (mTfR1-mAb-siSSB) or the DAR1 lipid AOC with the numerous different lipids attached at position 3 of the passenger strand (mTfR1-mAb-pos3.C22.siSSB, mTfR1-mAb-pos3.tzC14.siSSB, mTfR1-mAb-pos3.tzC16.siSSB, mTfR1-mAb-pos3.tzC18.siSSB, mTfR1-mAb-pos3.tzC20.siSSB, mTfR1-mAb-pos3.tzC18-oleyl.siSSB, mTfR1-mAb-pos3.tzC18-int.acid.siSSB) of the siRNA at Day 0.

The length of the lipid at position 3 of the passenger strand of lipid-AOCs was assessed for siRNA tissue concentrations. Most lipid AOCs with the different lipids were able to deliver higher amount of siRNAs to all 3 muscle tissues compared to the control AOCs at day 28 (FIGS. 10A-C). Among the lipid AOCs, the lipid AOCs with the C22 hydrocarbon chain (C22 lipid) (mTfR1-mAb-pos3.C22.siSSB) had the best activities in gastroc, TA, heart tissues. The lipid AOCs with C22 as the longest hydrocarbon chain (lipid) was able to deliver the highest amount of siRNA in the gastroc or TA with almost 9 nM, which is 10-fold more in gastroc and TA muscles (FIGS. 10A-B). In addition, the tzC20 (corresponding to 2′-O—C22) modified lipid AOC at position 3 produced the maximum tissue uptake. Interestingly, the lipid AOCs with the tz modified lipid tzC14 was not able to deliver higher amounts of siRNA compared to control AOCs. The C14, as the shortest lipid, was able to deliver lower amount of siRNAs to tissue at a concentration of 1.5 nM.

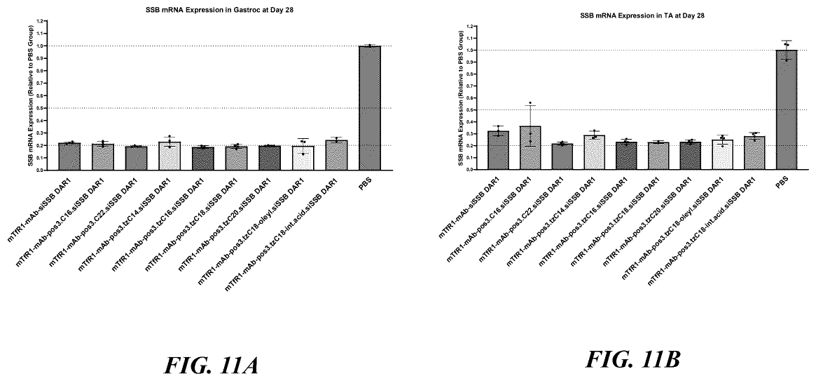

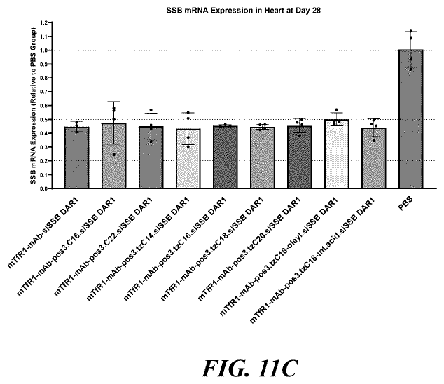

At Day 28, there were no significant differences observed in the reduction in mRNA expression levels for the lipid AOCs compared to control AOC (FIGS. 11A-C). At Day 28, all lipid AOCs and the control AOC reduce SSB mRNA expression levels to 20% in gastroc and 25% in TA, and to 50% in the heart.

Overall, the lipid AOCs and tz lipid modified AOCs were able to deliver higher amounts of siRNA in muscle cells. The AOC with longer lipid (tzC20 corresponding to 2′-O—C22) at position 3 were able to deliver higher levels of siRNA than lipid AOCs with shorter lipid. Interestingly higher tissue concentrations did not enhance the reduction in mRNA expression levels in tissues in these studies.

### Example 9: Lipid Length Optimization of 2′-O-Lipids at Passenger Strand Position 3 and Position 6 on siRNA DAR1 mAb AOCs in CD1 Mice

SSB: A 21-mer double-stranded siRNA with 19 bases of complementarity and 3′ dinucleotide overhangs was designed against mouse SSB. The sequence (5′ to 3′) of the guide/antisense strand was UUACAUUAAAGUCUGUUGUUU (SEQ ID NO: 5). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker.

### Conjugate Synthesis and Characterization

The CD71 mAb-siRNA DAR1 conjugates were made, purified and characterized as described in Example 1-3. All conjugates were made through cysteine conjugation using an MBS linker. Conjugates were characterized chromatographically as described in Table 20.

TABLE 20
HPLC Retention Time (RT) in Minutes

| | | RT, SAX | RT, SEC |
|---|---|---|---|
| Group(s) | Conjugate | Method-3 | Method-1 |
| 1-2 | mTfR1-mAb-siSSB DAR1 | 3.201 | 2.912 |
| 3-4 | mTfR1-mAb-pos3.C6.siSSB DAR1 | 3.091 | 2.933 |
| 5-6 | mTfR1-mAb-pos3.C10.siSSB DAR1 | 3.095 | 2.939 |
| 7-8 | mTfR1-mAb-pos3.C16.siSSB DAR1 | 3.103 | 2.947 |
| 9-10 | mTfR1-mAb-pos3.C22.siSSB DAR1 | 3.106 | 2.967 |
| 11-12 | mTfR1-mAb-pos6.C6.siSSB DAR1 | 3.204 | 2.913 |
| 13-14 | mTfR1-mAb-pos6.C10.siSSB DAR1 | 3.217 | 2.920 |
| 15-16 | mTfR1-mAb-pos6.C16.siSSB DAR1 | 3.244 | 2.951 |
| 17-18 | mTfR1-mAb-pos6.C22.siSSB DAR1 | 3.271 | 3.115 |

### In Vivo Study Design

The conjugates were assessed for their ability to mediate mRNA downregulation of SSB in skeletal muscle in vivo in wild type CD-1 mice. Mice were dosed via intravenous (IV) injection with PBS vehicle control or the indicated ASCs and at 0.5 mg/kg siRNA dose as shown in Table 21. After 28 days, gastrocnemius (gastroc), tibialis anterior (TA), liver, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 21
In vivo study design testing duration of action of SSB siRNA mAb DAR1 AOCs
containing 2′ lipid modifications at passenger strand position 3 or 6 in CD1 Mice

| | | | siRNA: | siRNA | | |
|---|---|---|---|---|---|---|
| | | | mAb Ratio | Dose | Doses | Harvest |
| Group | Test Article | N | (mol/mol) | (mg/kg) | (#) | (day) |
| 1 | mTfR1-mAb-siSSB DAR1 | 4 | 1 | 0.5 | 1 | 28 |
| 2 | mTfR1-mAb-siSSB DAR1 | 4 | 1 | 0.5 | 1 | 84 |
| 3 | mTfR1-mAb-pos3.C6.siSSB DAR1 | 4 | 1 | 0.5 | 1 | 28 |
| 4 | mTfR1-mAb-pos3.C6.siSSB DAR1 | 4 | 1 | 0.5 | 1 | 84 |
| 5 | mTfR1-mAb-pos3.C10.siSSB DAR1 | 4 | 1 | 0.5 | 1 | 28 |
| 6 | mTfR1-mAb-pos3.C10.siSSB DAR1 | 4 | 1 | 0.5 | 1 | 84 |
| 7 | mTfR1-mAb-pos3.C16.siSSB DAR1 | 4 | 1 | 0.5 | 1 | 28 |
| 8 | mTfR1-mAb-pos3.C16.siSSB DAR1 | 4 | 1 | 0.5 | 1 | 84 |
| 9 | mTfR1-mAb-pos3.C22.siSSB DAR1 | 4 | 1 | 0.5 | 1 | 28 |
| 10 | mTfR1-mAb-pos3.C22.siSSB DAR1 | 4 | 1 | 0.5 | 1 | 84 |
| 11 | mTfR1-mAb-pos6.C6.siSSB DAR1 | 4 | 1 | 0.5 | 1 | 28 |
| 12 | mTfR1-mAb-pos6.C6.siSSB DAR1 | 4 | 1 | 0.5 | 1 | 84 |
| 13 | mTfR1-mAb-pos6.C10.siSSB DAR1 | 4 | 1 | 0.5 | 1 | 28 |
| 14 | mTfR1-mAb-pos6.C10.siSSB DAR1 | 4 | 1 | 0.5 | 1 | 84 |
| 15 | mTfR1-mAb-pos6.C16.siSSB DAR1 | 4 | 1 | 0.5 | 1 | 28 |
| 16 | mTfR1-mAb-pos6.C16.siSSB DAR1 | 4 | 1 | 0.5 | 1 | 84 |
| 17 | mTfR1-mAb-pos6.C22.siSSB DAR1 | 4 | 1 | 0.5 | 1 | 28 |
| 18 | mTfR1-mAb-pos6.C22.siSSB DAR1 | 4 | 1 | 0.5 | 1 | 84 |
| 19 | PBS | 4 | N/A | N/A | 1 | 28 |
| 20 | PBS | 4 | N/A | N/A | 1 | 84 |

TABLE 22
siRNA Sequences used in Example 9

| | | | SEQ | | SEQ |
|---|---|---|---|---|---|
| Compound | siRNA | | ID | | ID |
| No. | Ref. | siRNA Guide Sequence | NO: | siRNA Passenger Sequence | NO: |
| 1 | siSSB | [5vp_una2moe](U)[ps].[fl2r] | 13 | [5am6_p_m](A)[ps].m(C)[ps].m(A)p. | 50 |
| | | (U)[ps].m(A)p.m(C)p.m(A)p. | | m(A)p.m(C)p.m(A)p.[fl2r](G)p.[f12r](A) | |
| | | [f12r](U)p.m(U)p.m(A)p.m(A) | | p.[fl2r](C)p.m(U)p.m(U)p.m(U)p.m(A)p. | |
| | | p.m(A)p.m(G)p.m(U)p.m(C)p. | | m(A)p.m(U)p.m(G)p.m(U)[ps].m(A)[ps]. | |
| | | [fl2r](U)p.m(G)p.[fl2r](U)p.m | | m(A) | |
| | | (U)p.m(G)p.m(U)[ps].m(U)[ps]. | | | |
| | | m(U) | | | |
| 2 | pos3.C | [5vp_una2moe](U)[ps].[fl2r | 13 | [5am6_p_m](A)[ps].m(C)[ps].[rc6](A) | 70 |
| | 6.siSS | (U)[ps].m(A)p.m(C)p.m(A)p. | | p.m(A)p.m(C)p.m(A)p.[fl2r](G)p.[fl2r] | |
| | B | [f12r](U)p.m(U)p.m(A)p.m(A) | | (A)p.[fl2r](C)p.m(U)p.m(U)p.m(U)p.m(A) | |
| | | p.m(A)p.m(G)p.m(U)p.m(C)p. | | p.m(A)p.m(U)p.m(G)p.m(U)[ps].m(A) | |
| | | [f12r](U)p.m(G)p.[fl2r](U)p.m | | [ps].m(A) | |
| | | (U)p.m(G)p.m(U)[ps].m(U)[ps | | | |
| | | 1.m(U) | | | |
| 3 | pos3.C | [5vp_una2moe](U)[ps].[fl2r] | 13 | [5am6_p_m](A)[ps].m(C)[ps].[rc10] | 71 |
| | 10.siS | (U)[ps].m(A)p.m(C)p.m(A)p. | | (A)p.m(A)p.m(C)p.m(A)p.[fl2r](G)p.[fl2r] | |
| | SB | [fl2r](U)p.m(U)p.m(A)p.m(A) | | (A)p.[fl2r](C)p.m(U)p.m(U)p.m(U)p.m | |
| | | p.m(A)p.m(G)p.m(U)p.m(C)p. | | (A)p.m(A)p.m(U)p.m(G)p.m(U)[ps].m(A) | |
| | | [f12r](U)p.m(G)p.[fl2r](U)p.m | | [ps].m(A) | |
| | | (U)p.m(G)p.m(U)[ps].m(U)[ps | | | |
| | | J.m(U) | | | |
| 4 | pos3.C | [5vp_una2moe](U)[ps].[f12r] | 13 | [5am6_p_m](A)[ps].m(C)[ps].[rc16]( | 52 |
| | 16.siS | (U)[ps].m(A)p.m(C)p.m(A)p. | | A)p.m(A)p.m(C)p.m(A)p.[fl2r](G)p.[f12r] | |
| | SB | [fl2r](U)p.m(U)p.m(A)p.m(A) | | (A)p.[fl2r](C)p.m(U)p.m(U)p.m(U)p.m | |
| | | p.m(A)p.m(G)p.m(U)p.m(C)p. | | (A)p.m(A)p.m(U)p.m(G)p.m(U)[ps].m(A) | |
| | | [fl2r](U)p.m(G)p.[fl2r](U)p.m | | [ps].m(A) | |
| | | (U)p.m(G)p.m(U)[ps].m(U)[ps | | | |
| | | 1.m(U) | | | |
| 5 | pos3.C | [5vp_una2moe](U)[ps].[fl2r] | 13 | [5am6_p_m](A)[ps].m(C)[ps].[rc22] | 63 |
| | 22.siS | (U)[ps].m(A)p.m(C)p.m(A)p. | | (A)p.m(A)p.m(C)p.m(A)p.[fl2r](G)p.[fl2r] | |
| | SB | [fl2r](U)p.m(U)p.m(A)p.m(A) | | (A)p.[fl2r](C)p.m(U)p.m(U)p.m(U)p.m | |
| | | p.m(A)p.m(G)p.m(U)p.m(C)p. | | (A)p.m(A)p.m(U)p.m(G)p.m(U)[ps].m(A) | |
| | | [f12r](U)p.m(G)p.[fl2r](U)p.m | | [ps].m(A) | |
| | | (U)p.m(G)p.m(U)[ps].m(U)[ps | | | |
| | | 1.m(U) | | | |
| 6 | pos6.C | [5vp_una2moe](U)[ps].[fl2r | 13 | [5am6_p_m](A)[ps].m(C)[ps].m(A)p. | 72 |
| | 6.siSS | ](U)[ps].m(A)p.m(C)p.m(A)p. | | m(A)p.m(C)p.[rc6](A)p.[f12r](G)p.[fl2r] | |
| | B | [f12r](U)p.m(U)p.m(A)p.m(A) | | (A)p.[fl2r](C)p.m(U)p.m(U)p.m(U)p.m(A) | |
| | | p.m(A)p.m(G)p.m(U)p.m(C)p. | | p.m(A)p.m(U)p.m(G)p.m(U)[ps].m(A) | |
| | | [f12r](U)p.m(G)p.[fl2r](U)p.m | | [ps].m(A) | |
| | | (U)p.m(G)p.m(U)[ps].m(U)[ps]. | | | |
| | | m(U) | | | |
| 7 | pos6.C | [5vp_una2moe](U)[ps].[fl2r] | 13 | [5am6_p_m](A)[ps].m(C)[ps].m(A)p. | 73 |
| | 10.siS | (U)[ps].m(A)p.m(C)p.m(A)p. | | m(A)p.m(C)p.[rc10](A)p.[fl2r](G)p.[f12r] | |
| | SB | [f12r](U)p.m(U)p.m(A)p.m(A) | | (A)p.[fl2r](C)p.m(U)p.m(U)p.m(U)p.m | |
| | | p.m(A)p.m(G)p.m(U)p.m(C)p. | | (A)p.m(A)p.m(U)p.m(G)p.m(U)[ps].m(A) | |
| | | [fl2r](U)p.m(G)p.[fl2r](U)p.m | | [ps].m(A) | |
| | | (U)p.m(G)p.m(U)[ps].m(U)[ps]. | | | |
| | | m(U) | | | |
| 8 | pos6.C | [5vp_una2moe](U)[ps].[fl2r] | 13 | [5am6_p_m](A)[ps].m(C)[ps].m(A)p. | 51 |
| | 16.siS | (U)[ps].m(A)p.m(C)p.m(A)p. | | m(A)p.m(C)p.[rc16](A)p.[fl2r](G)p.[fl2r] | |
| | SB | [f12r](U)p.m(U)p.m(A)p.m(A) | | (A)p.[fl2r](C)p.m(U)p.m(U)p.m(U)p.m | |
| | | p.m(A)p.m(G)p.m(U)p.m(C)p. | | (A)p.m(A)p.m(U)p.m(G)p.m(U)[ps].m(A) | |
| | | [f12r](U)p.m(G)p.[f12r](U)p.m | | [ps].m(A) | |
| | | (U)p.m(G)p.m(U)[ps].m(U)[ps | | | |
| | | 1.m(U) | | | |
| 9 | pos6.C | [5vp_una2moe](U)[ps].[fl2r] | 13 | [5am6_p_m](A)[ps].m(C)[ps].m(A)p. | 74 |
| | 22.siS | (U)[ps].m(A)p.m(C)p.m(A)p. | | m(A)p.m(C)p.[rc22](A)p.[fl2r](G)p.[fl2r] | |
| | SB | [fl2r](U)p.m(U)p.m(A)p.m(A) | | (A)p.[fl2r](C)p.m(U)p.m(U)p.m(U)p.m | |
| | | p.m(A)p.m(G)p.m(U)p.m(C)p. | | (A)p.m(A)p.m(U)p.m(G)p.m(U)[ps].m(A) | |
| | | [f12r](U)p.m(G)p.[fl2r](U)p.m | | [ps].m(A) | |
| | | (U)p.m(G)p.m(U)[ps].m(U)[ps | | | |
| | | J.m(U) | | | |

5vp_una2moe 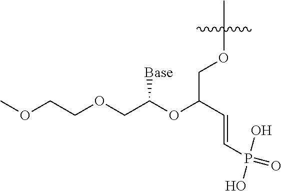
5am6 p_m 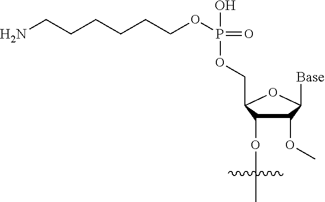
rc6 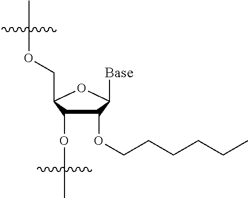
rc 10 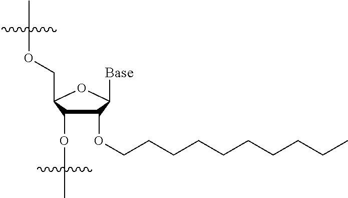
rc 16 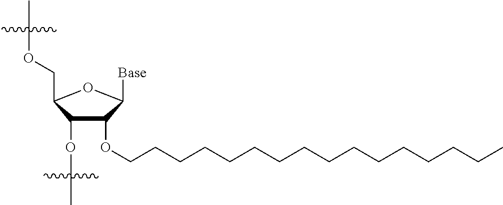
rc22

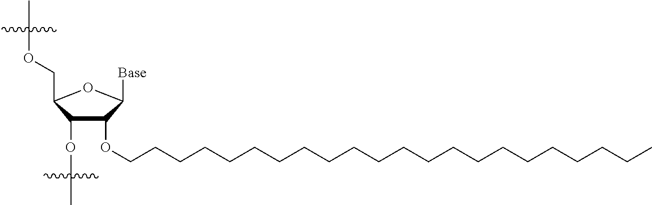

In Table 22, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

### Results

SSB siRNA tissue concentrations and SSB mRNA expression levels were assessed with DAR1 lipid-AOC with lipids of different lengths (C6, C10, C16, C22, TzC20) attached at position 3 or position 6 of the passenger strand at day 28 or day 84 in the gastroc, tibialis anterior (TA) and heart tissues obtained from mice following a single injection of a DAR1 control AOC (mTfR1-mAb-siSSB), the DAR1 lipid AOC with the numerous different lipids attached at 3 of the passenger strand (mTfR1-mAb-pos3.C6.siSSB, mTfR1-mAb-pos3.C10.siSSB, mTfR1-mAb-pos3.C16.siSSB, mTfR1-mAb-pos3.C22.siSSB) or the DAR1 lipid AOC with the numerous different lipids attached at 6 of the passenger strand (mTfR1-mAb-pos6.C6.siSSB, mTfR1-mAb-pos6.C10.siSSB, mTfR1-mAb-pos6.C16.siSSB, mTfR1-mAb-pos6.C22.siSSB) of the siRNA at Day 0.

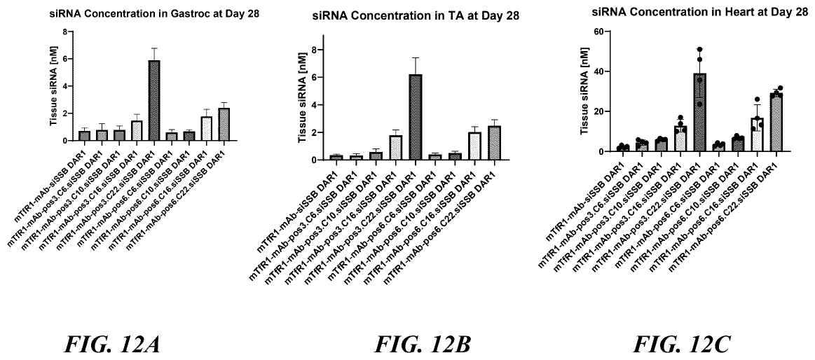

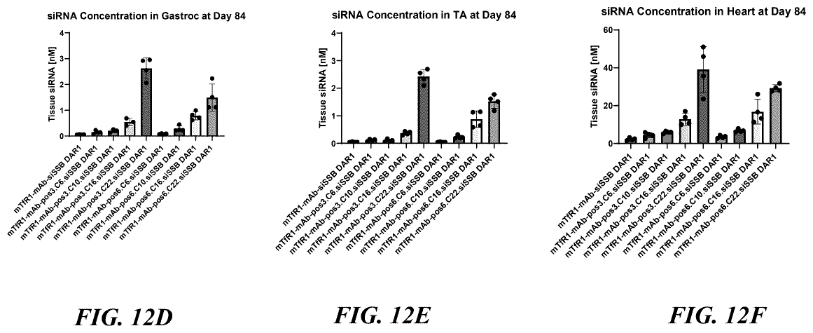

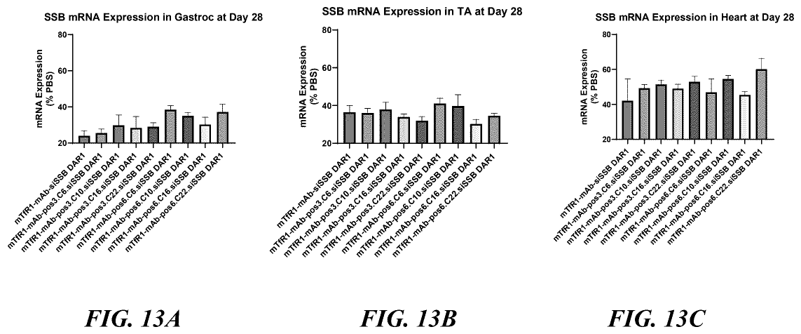

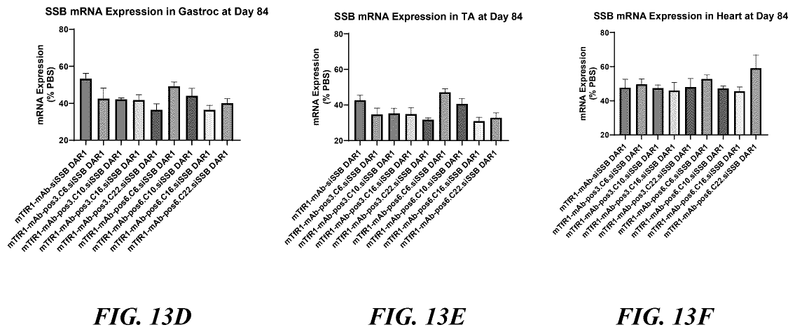

SSB siRNA tissue concentrations were assessed with DAR1 lipid-AOCs conjugated with lipid chains in different lengths in muscle tissue of mice at day 28 and day 84. The different lengths of the lipid conjugated at the position 3 or position 6 of the passenger strand of lipid AOCs were assessed for siRNA tissue concentrations (FIGS. 12A-F). At day 28, the lipid AOCs with C22 hydrocarbons at position 3 (mTfR1-mAb-pos3.C22.siSSB) as the longest lipid was able to deliver the highest amount of siRNA in all muscle tissues including in the gastroc or TA with almost 6 nM (FIGS. 12A-B) while the lipid AOCs with C22 at position 6 was able to deliver much lower siRNA levels to tissue at a concentration of around 2.5 nM, representing more than a fold increase in siRNA tissue concentrations. In addition, the lipid-AOCs with C22 at position 3 (mTfR1-mAb-pos3.C22.siSSB) delivered higher amounts of siRNA in tissues compared to the amounts of siRNAs by AOCs conjugated with the shorter lipid, C6, C10 or C16, at position 3 in all 3 muscle tissues. At day 84, the lipid-AOCs with C22 at position 3 was able to maintain its biological activities. At day 28, all DAR1 lipid AOCs at position 3 or position 6 did not show significant decrease in mRNA expression levels in all tissues compared to control AOCs (FIGS. 13-C). However, at day 84, the lipid AOCs with C22 at position 3 (mTfR1-mAb-pos3.C22.siSSB) showed the best activity among the lipid-AOCs with a reduction about 65-70% of SSB mRNA in gastroc or TA muscles (FIGS. 13D-E). In addition, the lipid-AOCs did not show significant increases in mRNA expression levels when compared to control AOC in the heart (FIG. 13F). Control AOCs mRNA expression levels decreased mRNA expression levels to the range of 50% to 60%.

Overall, the DAR1 lipid AOC with the C₂₂ lipid at position 3 of the passenger of the siRNA delivered high amount of siRNA to muscle tissues and induced sustained decreases in mRNA expression levels for an extended time.

### Example 10: Duration of Action of mTfR1-mAb-siMSTN DAR2 in CD1 Mice

MSTN: A 21-mer double-stranded siRNA with 19 bases of complementarity and 3′ dinucleotide overhangs was designed against mouse MSTN. The sequence (5′ to 3′) of the guide/antisense strand was UUAUUAUUUGUUCUUUGCCUU (SEQ ID NO: 4). Base, sugar and phosphate modifications were used to optimize the potency of the duplex and reduce immunogenicity. All single-stranded polynucleic acid molecules were fully assembled on solid phase using standard phosphoroamidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker.

### Conjugate Synthesis and Characterization

The CD71 mAb-siRNA DAR1 conjugates were made, purified and characterized as described in Examples 1-3. All conjugates were made through cysteine conjugation, a bismal linker. Conjugates were characterized chromatographically as described in Table 23.

TABLE 23
HPLC Retention Time (RT) in Minutes

| | | RT, SAX | RT, SEC |
|---|---|---|---|
| Group(s) | Conjugate | Method-5 | Method-1 |
| 1 | mTfR1-mAb-siMSTN DAR1 | 8.206 | 2.867 |
| 2-3 | mTfR1-mAb-siMSTN DAR2 | 8.884 | 2.763 |
| 4 | mTfR1-mAb-pos6.C16.siMSTN DAR1 | 8.269 | 2.895 |
| 5-6 | mTfR1-mAb-pos6.C16.siMSTN DAR2 | 9.023 | 2.817 |
| 7 | mTfR1-mAb-pos19.C16.siMSTN DAR1 | 8.390 | 3.011 |
| 8-9 | mTfR1-mAb-pos19.C16.siMSTN DAR2 | 10.566 | 3.021 |
| 10 | mTfR1-mAb-2x4PG.siMSTN DAR2 | 7.810 | 2.787 |
| 11 | mTfR1-mAb-2x4PG.pos6.C16.siMSTN DAR2 | 8.041 | 2.849 |

### In Vivo Study Design

The conjugates were assessed for their ability to mediate mRNA downregulation of myostatin (MSTN) in skeletal muscle in vivo in wild type CD-1 mice. Mice were dosed via intravenous (IV) injection with PBS vehicle control or the indicated ASCs and at 8 mg/kg mAb protein dose as shown in Table 24. After 28 days, gastrocnemius (gastroc), tibialis anterior (TA), liver, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 24
In vivo study design testing duration of action of mTfR1-mAb-siMSTN DAR2
AOCs in CD1 Mice

| | | | siRNA: | mAb | | |
|---|---|---|---|---|---|---|
| | | | mAb Ratio | Dose | Doses | Harvest |
| Group | Test Article | N | (mol/mol) | (mg/kg) | (#) | (day) |
| 1 | mTfR1-mAb-siMSTN DAR1 | 4 | 1 | 8 | 1 | 28 |
| 2 | mTfR1-mAb-siMSTN DAR2 | 4 | 2 | 8 | 1 | 2 |
| 3 | mTfR1-mAb-siMSTN DAR2 | 4 | 2 | 8 | 1 | 28 |
| 4 | mTfR1-mAb-pos6.C16.siMSTN DAR1 | 4 | 1 | 8 | 1 | 28 |
| 5 | mTfR1-mAb-pos6.C16.siMSTN DAR2 | 4 | 2 | 8 | 1 | 2 |
| 6 | mTfR1-mAb-pos6.C16.siMSTN DAR2 | 4 | 2 | 8 | 1 | 28 |
| 7 | mTfR1-mAb-pos19.C16.siMSTN DAR1 | 4 | 1 | 8 | 1 | 28 |
| 8 | mTfR1-mAb-pos19.C16.siMSTN DAR2 | 4 | 2 | 8 | 1 | 2 |
| 9 | mTfR1-mAb-pos19.C16.siMSTN DAR2 | 4 | 2 | 8 | 1 | 28 |
| 10 | mTfR1-mAb-2x4PG.siMSTN DAR2 | 4 | 2 | 8 | 1 | 28 |
| 11 | mTfR1-mAb-2x4PG.pos6.C16.siMSTN | 4 | 2 | 8 | 1 | 28 |
| | DAR2 | | | | | |
| 12 | PBS | 4 | N/A | N/A | 1 | 28 |

* All animals used in experiments shown in Table 24 were dosed with single dose by IV administration

TABLE 25
siRNA Sequences used in Example 10

| | | | SEQ | | SEQ |
|---|---|---|---|---|---|
| Compound | siRNA | | ID | | ID |
| No. | Ref. | siRNA Guide Sequence | NO: | siRNA Passenger Sequence | NO: |
| 1 | SIMSTN | [5vp_una2moe](U)[ps].[f12r](U) | 14 | [5am6_p_m](G)[ps].m(G)[ps].m(C)p. | 75 |
| | | [ps].m(A)p.m(U)p.m(U)p.[fl2r] | | m(A)p.m(A)p.m(A)p.[fl2r](G)p.[f12r] | |
| | | (A)p.m(U)p.m(U)p.m(U)p.m(G)p.m | | (A)p.[fl2r](A)p.m(C)p.m(A)p.m(A)p. | |
| | | (U)p.m(U)p.m(C)p.[fl2r](U)p. | | m(A)p.m(U)p.m(A)p.m(A)p.m(U)[ps]. | |
| | | m(U)p.[fl2r](U)p.m(G)p.m(C)p. | | m(A)[ps].m(A) | |
| | | m(C)[ps].m(U)[ps].m(U) | | | |
| 2 | pos6.C16. | [5vp_una2moe](U)[ps].[fl2r](U) | 14 | [5am6_p_m](G)[ps].m(G)[ps].m(C)p. | 76 |
| | siMSTN | [ps].m(A)p.m(U)p.m(U)p.[f12r] | | m(A)p.m(A)p.[rc16](A)p.[f12r](G)p.[f | |
| | | (A)p.m(U)p.m(U)p.m(U)p.m(G)p.m | | 12r](A)p.[fl2r](A)p.m(C)p.m(A)p.m(A) | |
| | | (U)p.m(U)p.m(C)p.[fl2r](U)p.m | | p.m(A)p.m(U)p.m(A)p.m(A)p.m(U) | |
| | | (U)p.[fl2r](U)p.m(G)p.m(C)p.m | | [ps].m(A)[ps].m(A) | |
| | | (C)[ps].m(U)[ps].m(U) | | | |
| 3 | pos19.C1 | [5vp_una2moe](U)[ps].[f12r](U) | 14 | [5am6_p_m](G)[ps].m(G)[ps].m(C)p. | 77 |
| | 6.siMST | [ps].m(A)p.m(U)p.m(U)p.[fl2r] | | m(A)p.m(A)p.m(A)p.[fl2r](G)p.[f12r] | |
| | N | (A)p.m(U)p.m(U)p.m(U)p.m(G)p.m | | (A)p.[fl2r](A)p.m(C)p.m(A)p.m(A)p. | |
| | | (U)p.m(U)p.m(C)p.[fl2r](U)p.m | | m(A)p.m(U)p.m(A)p.m(A)p.m(U)[ps] | |
| | | (U)p.[fl2r](U)p.m(G)p.m(C)p.m | | | |
| | | (C)[ps].m(U)[ps].m(U) | | | |
| 4 | 2x4PG.si | [5vp_una2moe](U)[ps].[f12r](U) | 15 | [5am6_p_m](G)[pg].m(G)[pg].m(C)p. | 78 |
| | MSTN | [pg].m(A)p.m(U)p.m(U)p.[fl2r] | | m(A)p.m(A)p.m(A)p.[fl2r](G)p.[f12r] | |
| | | (A)p.m(U)p.m(U)p.m(U)p.m(G) | | (A)p.[fl2r](A)p.m(C)p.m(A)p.m(A)p.m | |
| | | p.m(U)p.m(U)p.m(C)p.[fl2r](U)p.m | | (A)p.m(U)p.m(A)p.m(A)p.m(U)[pg].m | |
| | | (U)p.[fl2r](U)p.m(G)p.m(C)p.m | | (A)[pg].m(A) | |
| | | (C)[pg].m(U)[ps].m(U) | | | |
| 5 | 2x4PG.p | [5vp_una2moe](U)[ps].[f12r](U) | 15 | [5am6_p_m](G)[pg].m(G)[pg].m(C)p. | 79 |
| | os6.C16. | [pg].m(A)p.m(U)p.m(U)p.[fl2r] | | m(A)p.m(A)p.[rc16](A)p.[fl2r](G)p.[f | |
| | siMSTN | (A)p.m(U)p.m(U)p.m(U)p.m(G)p.m | | 12r](A)p.[fl2r](A)p.m(C)p.m(A)p.m(A) | |
| | | (U)p.m(U)p.m(C)p.[fl2r](U)p.m | | p.m(A)p.m(U)p.m(A)p.m(A)p.m(U) | |
| | | (U)p.[fl2r](U)p.m(G)p.m(C)p.m | | [pg].m(A)[pg].m(A) | |
| | | (C)[pg].m(U)[ps].m(U) | | | |

5vp_una2moe 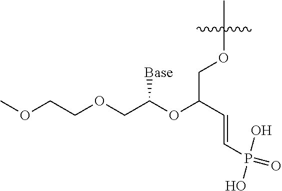
5am6_p_m 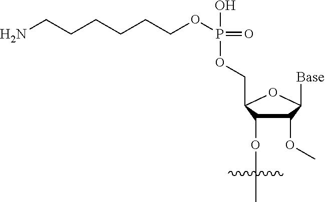
rc 16

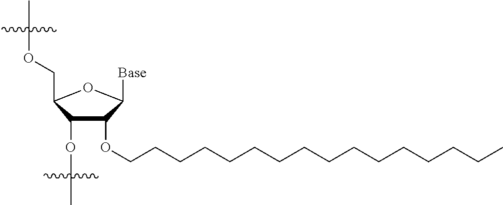

In Table 25, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage; [pg] indicates a phosphoryl guanidine (PG) linkage.

### Results

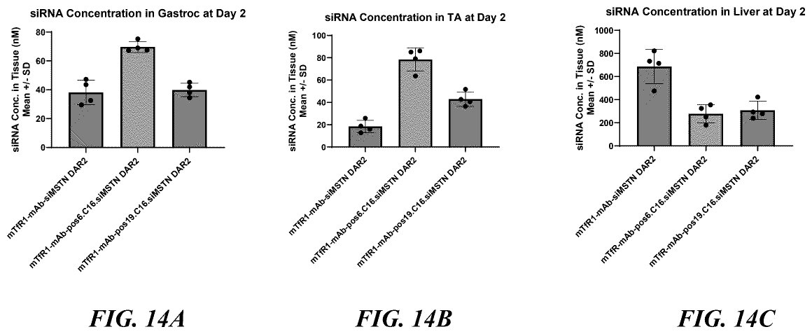

As shown in FIGS. 14A-14C, MSTN siRNA tissue concentrations were assessed with DAR 2 lipid-AOC with the C16 hydrocarbon chain conjugated at position 6 or position 19 of the passenger strand at day 2 in the gastroc (gastroc), tibialis anterior (TA), and liver tissues obtained from mice following a single injection of a DAR2 control AOC (mTfR1-mAb-siMSTN), the DAR2 lipid AOC with the C16 hydrocarbon chain conjugated at position 6 or position 19 of the passenger strand (mTfR1-mAb-pos6.C16.siMSTN, mTfR1-mAb-pos19.C16.siMSTN) of the siRNA at Day 0.

DAR2 AOCs with C16 hydrocarbon at position6 on the siRNA passenger strand was able to deliver higher amount of MSTN siRNA than the ones delivered the DAR2 C16 AOCs at position 19 in the gastroc or TA (FIGS. 14A-14B). However, the DAR2 lipid AOCs delivered much less MSTN siRNA in liver compared to the DAR2 control AOCs (FIG. 14C). Overall, at day 2 DAR2 C16 lipids AOCs were able to deliver higher amount of siRNAs to skeletal muscle than the amount of siRNAs delivered in non-muscle tissue.

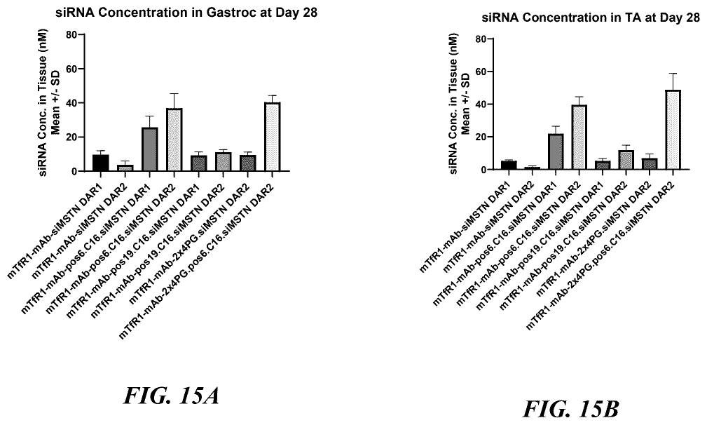

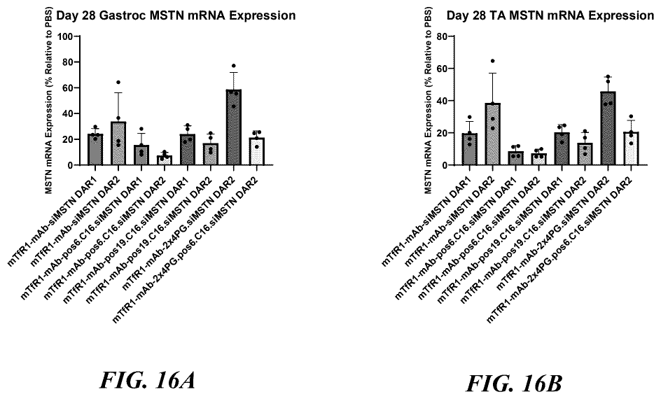

As shown in FIGS. 15A-15B and FIGS. 16A-16B, MSTN siRNA tissue concentrations and MSTN mRNA expression levels were assessed with DAR1/2 lipid AOC with the C16 hydrocarbon chain conjugated at position 6 or position 19 of the passenger strand at day 28 in the gastroc and anterior (TA) obtained from mice following a single injection of a DAR1 control AOC (mTfR1-mAb-siMSTN), the DAR1 lipid AOC with the C16 hydrocarbon chain conjugated at position 6 or position 19 of the passenger strand (mTfR1-mAb-pos6.C16.siMSTN DAR1, mTfR1-mAb-pos19.C16.siMSTN DAR1) or the DAR2 lipid AOC with the lipid C16 attached to position 6 or position 19 (mTfR1-mAb-pos6.C16.siMSTN DAR2, mTfR1-mAb-pos19.C16.siMSTN DAR2), or the DAR2 AOC PG modified oligonucleotide (mTfR1-mAb-2×4PG-siMSTN DAR2) or the DAR2 AOC PG modified oligonucleotide with the lipid C16 attached at 6 of the passenger strand (mTfR1-mAb-2×4PG.pos6.C16-siMSTN DAR2) of the siRNA at Day 0. mTfR1-mAb-2×4PG.pos6.C16-siMSTN is a mouse transferrin 1 receptor (mTfR1) mAb-MSTN siRNA conjugate, which i) sense/passenger strand is conjugated with C16 hydrocarbon chain at the nucleotide of position 6 from the 5′ end, and ii) guide strand comprises 2 phosphoguanidine (PG) modified internucleotide linkages, and passenger strand comprises 4 phosphoguanidine (PG) modified internucleotide linkages.

DAR1 lipid AOCs or DAR 2 lipid AOCs with the C16 hydrocarbon chain conjugated at position 6 could deliver much higher levels of siRNA tissue concentrations in gastroc and TA than the ones for DAR1 lipid AOCs or DAR 2 lipid AOCs with the C16 hydrocarbon chain conjugated at position 19 (FIG. 15A-15B). In addition, the DAR2 lipid AOC with siRNA modified with PG could deliver much higher siRNA than the DAR2 lipid AOC with unmodified siRNA.

As shown in FIGS. 16A-16B, DAR1 lipid AOCs or DAR 2 lipid AOCs with the C16 hydrocarbon chain conjugated at position 6 were able to induce much higher decreases in mRNA expression levels than the ones for DAR1 lipid AOCs or DAR 2 lipid AOCs with the C16 hydrocarbon chain conjugated at position 19. DAR 2 lipid AOCs with the C16 hydrocarbon chain conjugated at position 6 reduced mRNA expression levels by more than 90%. In addition, DAR2 lipid AOC with siRNA modified with PG was able to induce higher reduction of mRNA expression levels than the levels of DAR2 lipid AOCs with unmodified siRNA.

Overall, lipid AOCs with the C16 hydrocarbon chain conjugated at position 6 of the passenger strand of siRNA were able to deliver much higher amount of siRNA in muscle tissues and they had the best activities in reducing mRNA expression levels.

### Example 11: Plasma PK of mTfR1-mAb-siMSTN DAR2+ in CD1 Mice

MSTN: A 21-mer double-stranded siRNA with 19 bases of complementarity and 3′ dinucleotide overhangs was designed against mouse MSTN. The sequence (5′ to 3′) of the guide/antisense strand was UUAUUAUUUGUUCUUUGCCUU (SEQ ID NO: 4). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker.

### Conjugate Synthesis and Characterization

The CD71 mAb-siRNA conjugates were made, purified and characterized as described in Examples 1-3. All conjugates were made through cysteine conjugation, a SMCC linker. Conjugates were characterized chromatographically as described in Table 26.

TABLE 26
HPLC Retention Time (RT) in Minutes

| | | RT, SAX | |
|---|---|---|---|
| | | Method-3* | RT, SEC |
| Group(s) | Conjugate | Method-4\hat{ } | Method-1 |
| 1 | mTfR1-mAb-siMSTN DAR1 | 3.087* | 3.130 |
| 2 | mTfR1-mAb-pos6.C16.siMSTN DAR1 | 3.293* | 3.177 |
| 3 | mTfR1-mAb-siMSTN DAR2 | 3.470* | 3.066 |
| 4 | mTfR1-mAb-siMSTN, pos6.C16.siMSTN DAR2 | 3.493* | 2.885 |
| 5 | mTfR1-mAb-pos6.C16.siMSTN DAR2 | 3.701* | 3.117 |
| 6 | mTfR1-mAb-pos6.C16.siMSTN DAR4 | 11.205\hat{ } | 2.969 |
| 7 | mTfR1-mAb-pos6.C16.siMSTN DAR5 | 12.376\hat{ } | 2.609 |
| 8 | mTfR1-mAb-pos3.C22.siMSTN DAR1 | 3.300* | 3.293 |
| 9 | mTfR1-mAb-pos3.C22.siMSTN DAR2 | 3.725* | 3.094 |

### In Vivo Study Design

The conjugates were assessed for their plasma clearance and tissue distribution patterns 48 hours after intravenous injection. Mice were dosed via intravenous (IV) injection with PBS vehicle control or the indicated ASCs and at 6 mg/kg mAb protein dose as shown in Table 27. Blood was drawn 5 mins, 1 hr, 24 hrs, and 48 hrs after injection for determination of plasma clearance properties. After 2 days, gastrocnemius (gastroc), tibialis anterior (TA), liver, and heart muscle tissues were harvested and snap-frozen in liquid nitrogen. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 27
In vivo study design testing plasma clearance and tissue concentrations
at Day 2 of MSTN siRNA mAb AOCs in CD1 Mice

| | | siRNA | siRNA dose | mAb Dose | Harvest |
|---|---|---|---|---|---|
| Group(s) | Test Article | Target | (mg/kg) | (mg/kg) | (Day) |
| 1 | mTfR1-mAb-siMSTN DAR1 | MSTN | 0.56 | 6 | 2 |
| 2 | mTfR1-mAb-pos6.C16.siMSTN DAR1 | MSTN | 0.56 | 6 | 2 |
| 3 | mTfR1-mAb-siMSTN DAR2 | MSTN | 1.12 | 6 | 2 |
| 4 | mTfR1-mAb-siMSTN, pos6.C16.siMSTN DAR2 | MSTN | 1.12 | 6 | 2 |
| 5 | mTfR1-mAb-pos6.C16.siMSTN DAR2 | MSTN | 1.12 | 6 | 2 |
| 6 | mTfR1-mAb-pos6.C16.siMSTN DAR4 | MSTN | 2.24 | 6 | 2 |
| 7 | mTfR1-mAb-pos6.C16.siMSTN DAR5 | MSTN | 5.6 | 6 | 2 |
| 8 | mTfR1-mAb-pos3.C22.siMSTN DAR1 | MSTN | 0.56 | 6 | 2 |
| 9 | mTfR1-mAb-pos3.C22.siMSTN DAR2 | MSTN | 1.12 | 6 | 2 |

*All animals used in experiments shown in Table 27 were dosed with single dose by IV administration

TABLE 28
siRNA Sequences used in Example 11

| | | | SEQ | | SEQ |
|---|---|---|---|---|---|
| Compound | SiRNA | | ID | | ID |
| No. | Ref. | siRNA Guide Sequence | NO: | siRNA Passenger Sequence | NO: |
| 1 | siMSTN | [5vp_una2moe](U)[ps].[fl2r](U) | 14 | [5am6_p_m](G)[ps].m(G)[ps].m | 75 |
| | | [ps].m(A)p.m(U)p.m(U)p.[fl2r] | | (C)p.m(A)p.m(A)p.m(A)p.[f12r] | |
| | | (A)p.m(U)p.m(U)p.m(U)p.m(G)p.m | | (G)p.[fl2r](A)p.[fl2r](A)p.m(C)p.m | |
| | | (U)p.m(U)p.m(C)p.[fl2r](U)p.m | | (A)p.m(A)p.m(A)p.m(U)p.m | |
| | | (U)p.[fl2r](U)p.m(G)p.m(C)p.m | | (A)p.m(A)p.m(U)[ps].m(A)[ps]. | |
| | | (C)[ps].m(U)[ps].m(U) | | m(A) | |
| 2 | pos6.C16. | [5vp_una2moe](U)[ps].[fl2r](U) | 14 | [5am6_p_m](G)[ps].m(G)[ps].m | 76 |
| | siMSTN | [ps].m(A)p.m(U)p.m(U)p.[f12r] | | (C)p.m(A)p.m(A)p.[rc16](A)p.[f | |
| | | (A)p.m(U)p.m(U)p.m(U)p.m(G) | | 12r](G)p.[fl2r](A)p.[fl2r](A)p.m | |
| | | p.m(U)p.m(U)p.m(C)p.[f12r](U) | | (C)p.m(A)p.m(A)p.m(A)p.m(U)p.m | |
| | | p.m(U)p.[fl2r](U)p.m(G)p.m(C) | | (A)p.m(A)p.m(U)[ps].m(A)[p | |
| | | p.m(C)[ps].m(U)[ps].m(U) | | s].m(A) | |
| 3 | pos3.C22. | [5vp_una2moe](U)[ps].[fl2r](U) | 14 | [5am6_p_m](G)[ps].m(G)[ps].[r | 80 |
| | siMSTN | [ps].m(A)p.m(U)p.m(U)p.[fl2r] | | c22](C)p.m(A)p.m(A)p.m(A)p.[f | |
| | | (A)p.m(U)p.m(U)p.m(U)p.m(G) | | 12r](G)p.[fl2r](A)p.[f12r](A)p.m | |
| | | p.m(U)p.m(U)p.m(C)p.[fl2r](U) | | (C)p.m(A)p.m(A)p.m(A)p.m(U)p.m | |
| | | p.m(U)p.[fl2r](U)p.m(G)p.m(C) | | (A)p.m(A)p.m(U)[ps].m(A)[p | |
| | | p.m(C)[ps].m(U)[ps].m(U) | | s].m(A) | |

5vp_una2moe 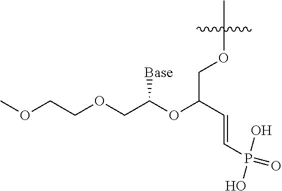
5am6_p_m 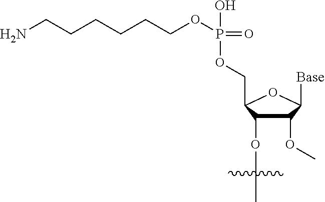
rc 16 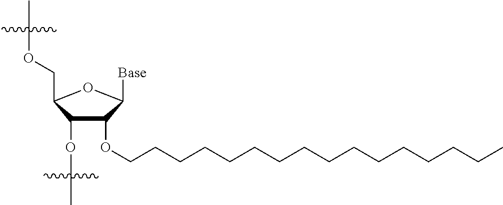
rc22

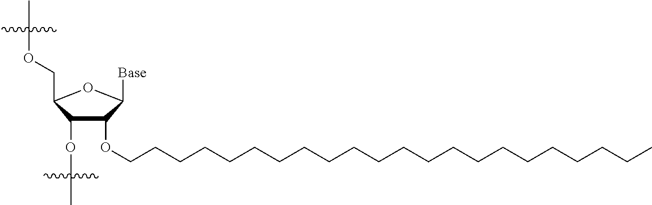

In Table 28, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

TABLE 29
Plasma PK of DAR1, DAR2, and DAR4 + 2′-O-C16 and
2′-O-C22 MSTN siRNA mAb AOCs in CD1 Mice

| AOC | Total Area | Std. Error |
|---|---|---|
| Non-lipid DAR1 | 702.2 | 38.95 |
| nonC16 DAR2 | 557.4 | 83.25 |
| C16 DAR1 | 255.7 | 85.33 |
| 1xC16 DAR2 | 148.5 | 32.85 |
| 2xC16 DAR2 | 27.58 | 8.045 |
| C16 DAR4 | 10.47 | 2.01 |
| C16 DAR5 | 12.42 | 1.284 |
| p3.C22 DAR1 | 191 | 45.8 |
| 2xp3.C22 DAR2 | 65.45 | 11.1 |

As shown in Table 29, the plasma pharmacokinetics (PK) of DAR1, DAR2, DAR4, or DAR5 lipid-AOCs with siRNA modified with passenger strand at position 6 with 2′-O—C16 and DAR1 or DAR2 lipid AOCs with siRNA modified with passenger strand position 3 with 2′-O—C22 were compared to non-lipid DAR1 and DAR2 AOCs in CD1 mice. All lipid AOCs had lower total area than the total of AOCs without a lipid moiety. For instance, the measured area for the non-lipid DAR1 AOC (non-lipid DAR1) was 702 while area for the DAR1 lipid AOC was 255 (C16 DAR1) as shown in Table 29. These changes in measured area indicated that the addition of the lipid to the lipid-modified AOCs facilitated rapid clearance from the plasma. In addition, an increase in DAR with lipid-AOCs correlated with a decrease of in the levels of DAR lipid AOCs in plasma. For instance, DAR4 lipid AOCs had a measured area of 10.5 while the area for the DAR2 lipid AOCs was 148.

Overall, these results indicated that the lipid AOCs are rapidly cleared from the plasma and that the plasma clearance correlates with the DAR of the lipid AOCs.

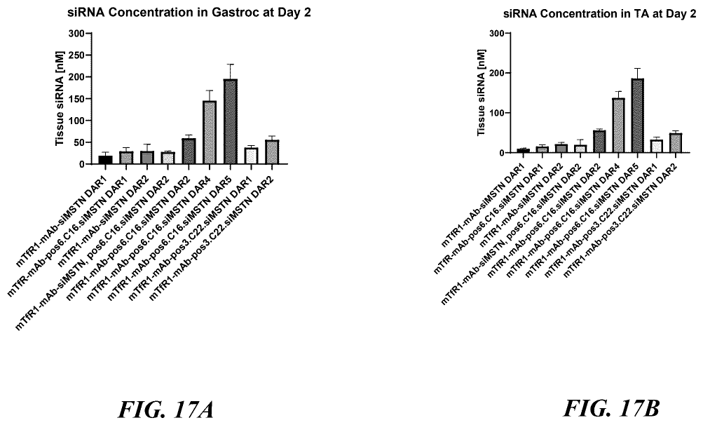

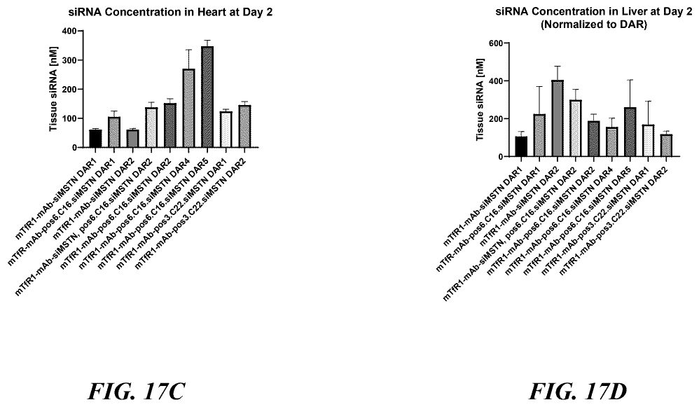

As shown in FIGS. 17A-17D, MSTN siRNA tissue concentrations were assessed with DAR1/2/4/5 lipid AOC with the C16 hydrocarbon conjugated at position 6 or with the lipid C22 attached at position 3 of the passenger strand at day 2 in the gastroc, tibialis anterior (TA), heart, and liver tissues obtained from mice following a single injection of a DAR1 control AOC (mTfR1-mAb-siMSTN DAR1) or DAR2 control (mTfR1-mAb-siMSTN DAR2), the DAR1 lipid AOC with the C16 hydrocarbon conjugated at position 6 of the passenger strand (mTfR1-mAb-pos6.C16.siMSTN DAR1mTfR1), the DAR2/4/5 lipid AOC with the C16 hydrocarbon conjugated to position 6 (mTfR1-mAb-pos6.C16.siMSTN DAR2/4/5), the DAR1/2 lipid AOC with the C22 hydrocarbon conjugated to position 3 of the passenger strand (mTfR1-mAb-pos3.C22.siMSTN DAR1/2) of the siRNA at Day 0.

At Day 2 DAR4 lipid AOCs or DAR5 lipid AOCs with C16 hydrocarbon conjugated at position 6 were able to deliver higher amount of MSTN siRNA in all tested muscle tissues (gastroc, TA, and heart) than the levels delivered by the DAR1/2 lipid AOCs (FIGS. 17A-17C). In the gastroc and TA, DAR5 lipid AOCs with C16 hydrocarbon conjugated at position 6 delivers almost 200 nM of siRNA, which is more than a 6-fold increase over the DAR1/2 lipid (FIGS. 17A-B). However, the DAR4 lipid AOCs and DAR5 lipid AOCs with C16 hydrocarbon conjugated at position 6 did not show significant differences in MSTN tissue concentrations over the DAR1/2 lipid AOCs in the liver (FIG. 17D).

Overall, the DAR4/5 lipid AOCs were able to deliver the most amount of siRNA to muscle tissue 2 days following injections of these lipid AOCs despite rapid clearance from the plasma.

### Example 12: Duration of Action of mTfR1-mAb-siMSTN DAR2+ in CD1 Mice

MSTN: A 21-mer double-stranded siRNA with 19 bases of complementarity and 3′ dinucleotide overhangs was designed against mouse MSTN. The sequence (5′ to 3′) of the guide/antisense strand was UUAUUAUUUGUUCUUUGCCUU (SEQ ID NO: 4). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double stranded siRNA. The passenger strand contained a conjugation handle, a C6-NH2, at the 5′ end, which was connected to the siRNA passenger strand via phosphorothioate-inverted abasic-phosphorothioate linker. The passenger strand was modified with 2′-O—C16 at position 6 or 2′-O—C22 at position 3.

### Conjugate Synthesis and Characterization

The CD71 mAb-siRNA conjugates were made, purified and characterized as described in Examples 1-3. All conjugates were made through cysteine conjugation, a SMCC linker. Conjugates were characterized chromatographically as described in Table 30.

TABLE 30
HPLC Retention Time (RT) in Minutes

| | | RT, SAX | |
|---|---|---|---|
| | | Method-3* | RT, SEC |
| Group | Conjugate | Method-4\hat{ } | Method-1 |
| 1 | mTfR1-mAb-siMSTN DAR1 | 3.087* | 3.130 |
| 2 | mTfR1-mAb-pos6.C16.siMSTN DAR1 | 3.293* | 3.177 |
| 3 | mTfR1-mAb-siMSTN DAR2 | 3.470* | 3.066 |
| 4 | mTfR1-mAb-siMSTN, pos6.C16.siMSTN DAR2 | 3.493* | 2.885 |
| 5 | mTfR1-mAb-pos6.C16.siMSTN DAR2 | 3.701* | 3.117 |
| 6 | mTfR1-mAb-pos6.C16.siMSTN DAR4 | 11.205\hat{ } | 2.969 |
| 7 | mTfR1-mAb-pos6.C16.siMSTN DAR5 | 12.376\hat{ } | 2.609 |
| 8 | mTfR1-mAb-pos3.C22.siMSTN DAR1 | 3.300* | 3.293 |
| 9 | mTfR1-mAb-pos3.C22.siMSTN DAR2 | 3.725* | 3.094 |

### In Vivo Study Design

The conjugates were assessed for their ability to mediate mRNA downregulation of myostatin (MSTN) in skeletal muscle in vivo in wild type CD-1 mice. Mice were dosed via intravenous (IV) injection with PBS vehicle control or the indicated ASCs and at 6 mg/kg mAb protein dose as shown in Table 31. After 28 days, gastrocnemius (gastroc), tibialis anterior (TA), liver, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 31
In vivo study design testing duration of action of MSTN siRNA mAb DAR2 AOCs in CD1 Mice

| | | siRNA dose | mAb Dose | Doses | Harvest |
|---|---|---|---|---|---|
| Group | Test Article | (mg/kg) | (mg/kg) | (#) | (Day) |
| 1 | mTfR1-mAb-siMSTN DAR1 | 0.56 | 6 | 1 | 28 |
| 2 | mTfR1-mAb-pos6.C16.siMSTN DAR1 | 0.56 | 6 | 1 | 28 |
| 3 | mTfR1-mAb-siMSTN DAR2 | 1.12 | 6 | 1 | 28 |
| 4 | mTfR1-mAb-siMSTN, pos6.C16.siMSTN DAR2 | 1.12 | 6 | 1 | 28 |
| 5 | mTfR1-mAb-pos6.C16.siMSTN DAR2 | 1.12 | 6 | 1 | 28 |
| 6 | mTfR1-mAb-pos6.C16.siMSTN DAR4 | 2.24 | 6 | 1 | 28 |
| 7 | mTfR1-mAb-pos6.C16.siMSTN DAR5 | 5.6 | 6 | 1 | 28 |
| 8 | mTfR1-mAb-pos3.C22.siMSTN DAR1 | 0.56 | 6 | 1 | 28 |
| 9 | mTfR1-mAb-pos3.C22.siMSTN DAR2 | 1.12 | 6 | 1 | 28 |

TABLE 32
siRNA Sequences used in Example 12

| | | | SEQ | | SEQ |
|---|---|---|---|---|---|
| Compound | SIRNA | | ID | | ID |
| No. | Ref. | siRNA Guide Sequence | NO: | siRNA Passenger Sequence | NO: |
| 1 | siMSTN | [5vp_una2moe](U)[ps].[f12r] | 14 | [5am6_p_m](G)[ps].m(G)[ps]. | 75 |
| | | (U)[ps].m(A)p.m(U)p.m(U)p.[fl | | m(C)p.m(A)p.m(A)p.m(A)p.[fl | |
| | | 2r](A)p.m(U)p.m(U)p.m(U)p.m | | 2r](G)p.[fl2r](A)p.[fl2r](A)p.m | |
| | | (G)p.m(U)p.m(U)p.m(C)p.[fl | | (C)p.m(A)p.m(A)p.m(A)p.m | |
| | | 2r](U)p.m(U)p.[fl2r](U)p.m(G) | | (U)p.m(A)p.m(A)p.m(U)[ps].m | |
| | | p.m(C)p.m(C)[ps].m(U)[ps]. | | (A)[ps].m(A) | |
| | | m(U) | | | |
| 2 | pos6.C16.s | [5vp_una2moe](U)[ps].[f12r] | 14 | [5am6_p_m](G)[ps].m(G)[ps]. | 76 |
| | IMSTN | (U)[ps].m(A)p.m(U)p.m(U)p.[fl | | m(C)p.m(A)p.m(A)p.[rc16](A) | |
| | | 2r](A)p.m(U)p.m(U)p.m(U)p. | | p.[fl2r](G)p.[fl2r](A)p.[fl2r](A) | |
| | | m(G)p.m(U)p.m(U)p.m(C)p.[fl | | p.m(C)p.m(A)p.m(A)p.m(A)p | |
| | | 2r](U)p.m(U)p.[fl2r](U)p.m(G) | | .m(U)p.m(A)p.m(A)p.m(U)[ps]. | |
| | | p.m(C)p.m(C)[ps].m(U)[ps]. | | m(A)[ps].m(A) | |
| | | m(U) | | | |
| 3 | pos3.C22.s | [5vp_una2moe](U)[ps].[fl2r] | 14 | [5am6_p_m](G)[ps].m(G)[ps]. | 80 |
| | IMSTN | (U)[ps].m(A)p.m(U)p.m(U)p.[fl | | [rc22](C)p.m(A)p.m(A)p.m(A) | |
| | | 2r](A)p.m(U)p.m(U)p.m(U)p. | | p.[fl2r](G)p.[f12r](A)p.[fl2r](A) | |
| | | m(G)p.m(U)p.m(U)p.m(C)p.[fl | | p.m(C)p.m(A)p.m(A)p.m(A)p.m | |
| | | 2r](U)p.m(U)p.[fl2r](U)p.m(G) | | (U)p.m(A)p.m(A)p.m(U)[ps].m | |
| | | p.m(C)p.m(C)[ps].m(U)[ps]. | | (A)[ps].m(A) | |
| | | m(U) | | | |

5vp_una2moe 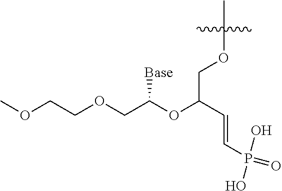
5am6_p_m 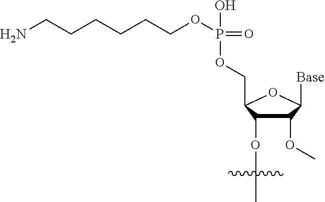
rc 16 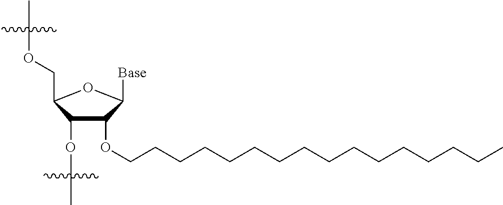
rc22

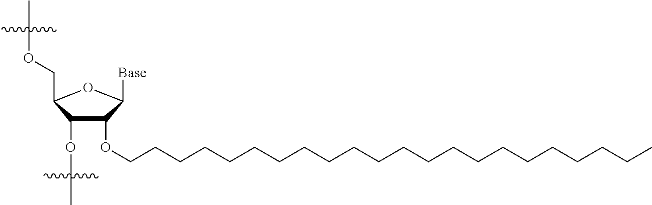

In Table 32, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

### Results

The DAR 1/2/5 lipid-AOC with the C16 hydrocarbon conjugated to the passenger strand at position 6 (mTfR1-mAb.pos6.C16.siMSTN DAR1, mTfR1-mAb.pos6.C16.siMSTN DALR2, mTfR1-mAb.pos6.C16.siMSTN DALR5) were reduced and analyzed by using a CE-SDS.

TABLE 33
ELISA binding affinity of DAR1 AOC and DAR2 C16 lipid AOC

| | | | | R | Rel pot |
|---|---|---|---|---|---|
| Conjugate | Bₘₐₓ | h | K_{d} | squared | % |
| 1. rIgG2c.R17217.mAb | 3.262 | 1.01 | 135.5 | 0.9989 | 100.00 |
| 2. rIgG2c.R17217.mAb-MCC- | 3.193 | 1.056 | 150 | 1 | 90.33 |
| MSTN 7f.8s DAR1 | | | | | |
| 3. rIgG2c.R17217.mAb-MCC- | 3.239 | 1.09 | 144.2 | 0.9991 | 93.97 |
| MSTN 7f.8s + p6.C16 DAR1 | | | | | |

TABLE 34
ELISA binding affinity of DAR2 AOC and DAR2 C16 lipid AOC

| | | | | R | Rel pot |
|---|---|---|---|---|---|
| Conjugate | Bₘₐₓ | h | K_{d} | squared | % |
| 1. rIgG2c.R17217.mAb | 3.235 | 1.032 | 122.3 | 0.9974 | 100.00 |
| 4. rIgG2c.R17217.mAb-MCC- | 3.242 | 1.038 | 164 | 0.9996 | 74.57 |
| MSTN 7f.8s DAR2 | | | | | |
| 5. rIgG2c.R17217.mAb-MCC- | 3.252 | 1.046 | 157.5 | 0.9996 | 77.65 |
| MSTN 7f.8s + 1 PS p6.C16, | | | | | |
| 1 PS 7f.8s DAR2 | | | | | |

TABLE 35
ELISA binding affinity of DAR2 C16 lipid AOC

| | | | | R | Rel pot |
|---|---|---|---|---|---|
| Conjugate | Bₘₐₓ | h | K_{d} | squared | % |
| 1. rIgG2c.R17217.mAb | 3.424 | 1.015 | 114.8 | 0.9985 | 100.00 |
| 6. rIgG2c.R17217.mAb-MCC- | 3.295 | 1.069 | 132.1 | 0.9997 | 86.90 |
| MSTN 7f.8s + p6.C16 DAR2 | | | | | |

TABLE 36
ELISA binding affinity of DAR4 and DAR5 C16 lipid AOC

| | | | | R | Rel pot |
|---|---|---|---|---|---|
| Conjugate | Bₘₐₓ | h | K_{d} | squared | % |
| 1. rIgG2c.R17217.mAb | 3.494 | 1.142 | 89.66 | 0.9997 | 100.00 |
| 7. rIgG2c.R17217.mAb- | 3.603 | 1.13 | 131.8 | 0.9999 | 68.03 |
| MCC-MSTN 7f.8s + | | | | | |
| p6.C16 DAR4 | | | | | |
| 8. rIgG2c.R17217.mAb- | 3.504 | 1.123 | 129.5 | 0.9998 | 69.24 |
| MCC-MSTN 7f.8s + | | | | | |
| p6.C16 DAR6 | | | | | |

TABLE 37
ELISA binding affinity of DAR1 and DAR2 C22 lipid AOC

| | | | | R | Rel pot |
|---|---|---|---|---|---|
| Conjugate | Bₘₐₓ | h | K_{d} | squared | % |
| 1. rIgG2c.R17217.mAb | 3.6 | 1.065 | 100.2 | 0.9999 | 100.00 |
| 9. rIgG2c.R17217.mAb- | 3.743 | 1.018 | 98.94 | 0.9998 | 101.27 |
| MCC-MSTN 7f.8s + | | | | | |
| p3.C22 DAR1 | | | | | |
| 10. rIgG2c.R17217.mAb- | 3.637 | 1.059 | 122.9 | 0.9994 | 81.53 |
| MCC-MSTN 7f.8s + | | | | | |
| p3.C22 DAR2 | | | | | |

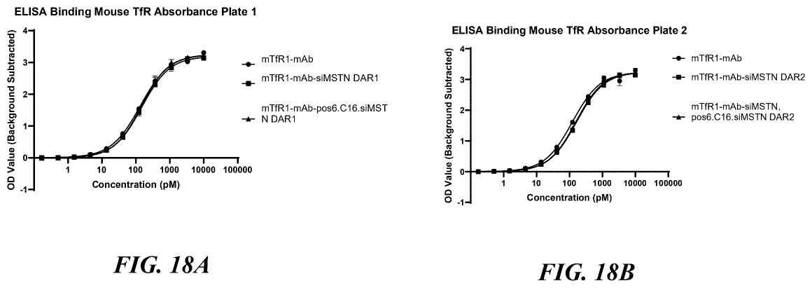

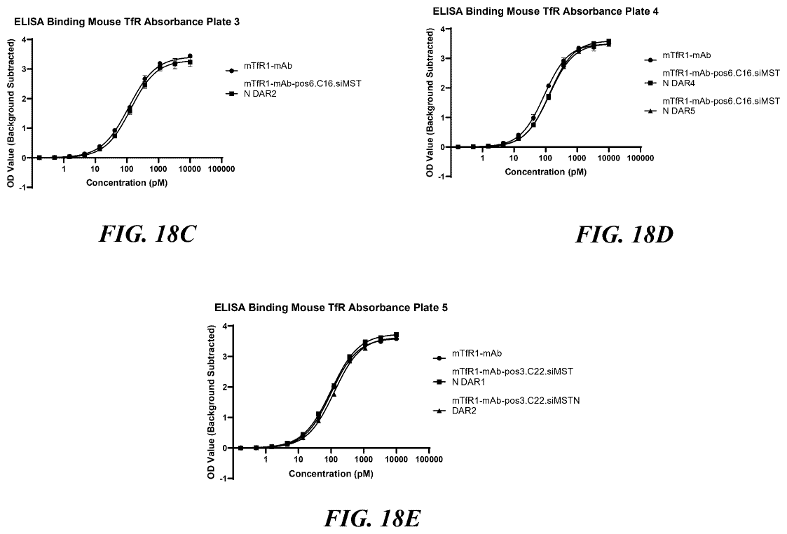

As shown in FIGS. 18A-18E and Tables 33-37, TfR1 mAb AOCs with DAR1, DAR2, DAR4, and DAR5 containing siRNA modified with 2′-O—C16 at position 6 nucleotide of the passenger strand and mTfR1 mAb AOCs with DAR1 and DAR2 containing siRNA modified with 2′-O—C22 at position 3 of the passenger strand were compared to a non-lipid mTfR1 mAb AOCs with DAR1 and DAR2.

Analysis of ELISA binding affinities of the mAb of AOCs and the mAb of lipid AOCs showed that binding affinities of the mAb with DAR1/2/4/5 lipid AOCs with the C16 lipid conjugated to the passenger strand at position 6 (mTfR1-mAb.pos6.C16.siMSTN DAR1, mTfR1-mAb.pos6.C16.siMSTN DAR2, mTfR1-mAb.pos6.C16.siMSTN DAR4, mTfR1-mAb.pos6.C16.siMSTN DAR5) or mAb with DAR1/2 lipid AOC with the C22 lipid conjugated to position 3 of the passenger strand (mTfR1-mAb.pos3.C22.siMSTN DAR1, mTfR1-mAb.pos3.C22.siMSTN DAR2) remained the same as the control antibody (mTfR1-mAb) or the mAb of the control AOC, the monoclonal antibody conjugated with the DMPK siRNA (mTfR1-mAb.siMSTN). The results indicated that the siRNA modified with the lipid C16 conjugated at position 6 nucleotide or lipid C22 conjugated at position 3 nucleotide of the passenger strand of the siRNA conjugated to the anti-mTfR1 antibody do not affect the receptor binding affinity of the antibody when compared to mTfR1 antibody. More importantly, these results showed that affinities of the mouse anti-transferrin receptor antibody of the AOCs were not significantly impacted by addition of up to an average of 5 lipid-modified siRNAs to the mAb (DAR5 lipid AOCs).

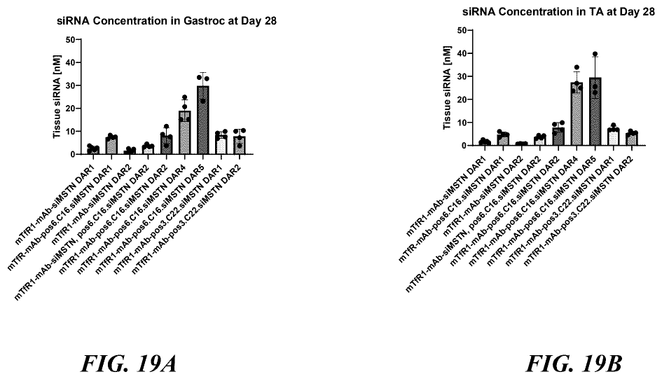

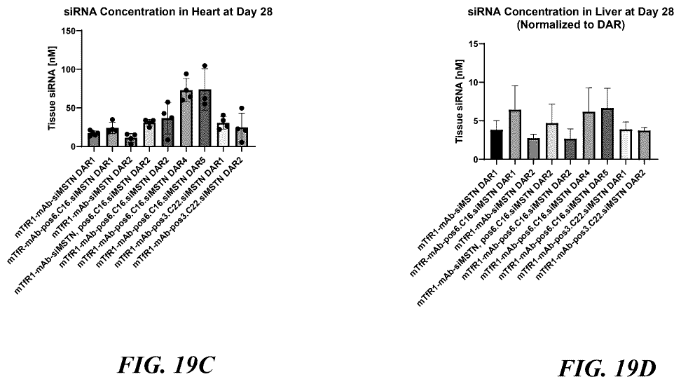

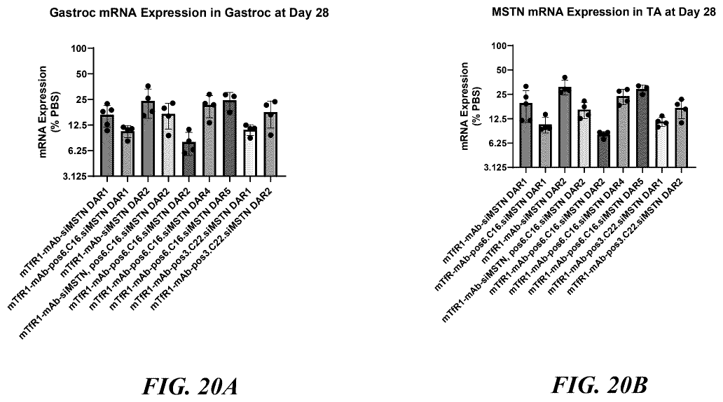

As shown in FIGS. 19A-19D and FIGS. 20A-20B, MSTN siRNA tissue concentrations and MSTN mRNA expression levels were assessed with DAR1/2/4/5 lipid AOC with the C16 hydrocarbon chain conjugated at position 6 or with the C22 hydrocarbon chain conjugated at position 3 of the passenger strand at day 28 in the gastroc, tibialis anterior (TA), heart, and liver obtained from mice following a single injection of a DAR1 control AOC (mTfR1-mAb-siMSTN DAR1) or DAR2 control (mTfR1-mAb-siMSTN DAR2), the DAR1 lipid AOC with the C16 hydrocarbon chain conjugated at position 6 of the passenger strand (mTfR1-mAb-pos6.C16.siMSTN DAR1mTfR1−), the DAR2/4/5 lipid AOC with the lipid C16 attached to position 6 (mTfR1-mAb-pos6.C16.siMSTN DAR2/4/5), the DAR1/2 lipid AOC with the C22 hydrocarbon chain conjugated at position 3 of the passenger strand (mTfR1-mAb-pos3.C22.siMSTN DAR1/2) of the siRNA at Day 0.

At day 28 MSTN siRNA tissue concentrations were assessed with DAR1/2/4/5 lipid AOCs with C16 conjugated at position 6 of the passenger strand in muscle tissue of mice. As shown in FIGS. 19A-C, DAR4 lipid AOCs and DAR5 lipid AOCs with C16 conjugated at position 6 were able to deliver higher amount of MSTN siRNA than that of the DAR1/2 lipid AOCs in muscle tissues (gastroc, TA and heart). For instance, DAR5 lipid AOCs with C16 conjugated at position 6 delivered almost 30 nM of MSTN siRNA in the gastroc, which was more than a 3-fold increase over the DAR1/2 lipid AOCs (~8 nM of MSTN siRNA) including the DAR1/2 lipid AOCs with C22 conjugated at position 3 of the passenger strand (FIG. 19A). However, the DAR4 lipid AOCs and DAR5 lipid AOCs with C16 conjugated at position 6 did not show significant differences in MSTN tissue concentrations over the DAR1/2 lipid AOCs in the liver (FIG. 19D).

At day 28 mRNA expression levels in tissue were measured in tissue obtained from mice that were administered a single injection of DAR1/2/4/5 lipid AOCs with C16 conjugated at position 6 of the passenger strand in muscle tissue of mice. All lipid OACs had over 75% reduction in mRNA expression levels and the DAR2 lipid AOCs with C16 conjugated at position 6 had the best activity with over 90% reduction in mRNA expression levels (FIGS. 20A-20B).

Overall, the DAR4/5 lipid AOCs were able to deliver the most amount of siRNA to muscle tissues. Although the DAR2 lipid AOCs with C16 conjugated at position 6 delivered a modest amount of siRNA compared to the DAR4/5 lipid AOCs, it had the best activity with over 90% reduction in target mRNA expression levels.

### Example 13: Duration of Action of Human TfR1 Targeted C16 Fab siRNA Conjugates (FabOC) in Humanized TfR1 Mice

DMPK: A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The passenger strand was modified with 2′-O—C16 at position 6.

### Conjugate Synthesis and Characterization

The CD71 Fab-siRNA conjugates were made, purified and characterized as described in Examples 1-3. All conjugates were made through cysteine conjugation, a SMCC linker. Conjugates were characterized chromatographically as described in Table 38.

TABLE 38
HPLC Retention Time (RT) in Minutes

| | | RT, SAX | RT, SEC |
|---|---|---|---|
| Group(s) | Conjugate | Method-3 | Method-2 |
| 1 + 5 | hTfR1-Fab-siDMPK DAR1 | 3.718 | 15.598 |
| 2 + 6 | hTfR1-Fab-siDMPK DAR2 | 4.157 | 14.315 |
| 3 + 7 | hTfR1-Fab-pos6.C16.siDMPK DAR1 | 3.719 | 16.708 |
| 4 + 8 | hTfR1-Fab-pos6.C16.siDMPK DAR2 | 4.134 | 15.350 |

### In Vivo Study Design

The conjugates were assessed for their ability to mediate mRNA downregulation of myotonic dystrophy protein kinase (DMPK) in skeletal muscle in vivo in humanized TfR1 mice. Mice were dosed via intravenous (IV) injection or subcutaneous (SC) with PBS vehicle control or the indicated AOCs and at 2.14 mg/kg mAb protein dose, or 0.6 mg/kg for the DAR1 conjugates and 1.2 mg/kg for the DAR2 conjugates as shown in Table 39. After 28 days, gastrocnemius (gastroc), tibialis anterior (TA), liver, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 39
In vivo study design testing duration of action of DMPK siRNA Fab AOCs in
humanized TfR1 Mice

| | | siRNA | Fab | | | | |
|---|---|---|---|---|---|---|---|
| | | dose | Dose | Doses | Dose | | Harvest |
| Group | Test Article | (mg/kg) | (mg/kg) | (#) | Schedule | ROA | (Day) |
| 1 | hTfR1-Fab-siDMPK DAR1 | 0.6 | 2.14 | 1 | single | IV | 28 |
| 2 | hTfR1-Fab-siDMPK DAR2 | 1.2 | 2.14 | 1 | single | IV | 28 |
| 3 | hTfR1-Fab- | 0.6 | 2.14 | 1 | single | IV | 28 |
| | pos6.C16.siDMPK DAR1 | | | | | | |
| 4 | hTfR1-Fab- | 1.2 | 2.14 | 1 | single | IV | 28 |
| | pos6.C16.siDMPK DAR2 | | | | | | |
| 5 | hTfR1-Fab-siDMPK DAR1 | 0.6 | 2.14 | 1 | single | SC | 28 |
| 6 | hTfR1-Fab-siDMPK DAR2 | 1.2 | 2.14 | 1 | single | SC | 28 |
| 7 | hTfR1-Fab- | 0.6 | 2.14 | 1 | single | SC | 28 |
| | pos6.C16.siDMPK DAR1 | | | | | | |
| 8 | hTfR1-Fab- | 1.2 | 2.14 | 1 | single | SC | 28 |
| | pos6.C16.siDMPK DAR2 | | | | | | |
| 9 | PBS | N/A | N/A | 1 | single | SC | 28 |

TABLE 40
siRNA Sequences used in Example 13

| | | | SEQ | | SEQ |
|---|---|---|---|---|---|
| Compound | | | ID | | ID |
| No. | siRNA Ref. | siRNA Guide Sequence | NO: | siRNA Passenger Sequence | NO: |
| 1 | siDMPK | [5vp_una2moe](U)[ps].[fl2r](A) | 12 | [5am6_p_m](C)[ps].m(C)[ps]. | 48 |
| | | [ps].m(A)p.m(U)p.m(A)p.[fl2r] | | m(G)p.m(A)p.m(C)p.m(A)p.[fl | |
| | | (C)p.m(C)p.m(G)p.m(A)p.m(G) | | 2r](U)p.[fl2r](U)p.[fl2r](C)p.m | |
| | | p.m(G)p.m(A)p.m(A)p.[fl2r](U) | | (C)p.m(U)p.m(C)p.m(G)p.m(G) | |
| | | p.m(G)p.[fl2r](U)p.m(C)p.m(G) | | p.m(U)p.m(A)p.m(U)[ps].m(U) | |
| | | p.m(G)[ps].m(U)[ps].m(U) | | [ps].m(A) | |
| 2 | pos6.C16.siD | [5vp_una2moe](U)[ps].[fl2r](A) | 12 | [5am6_p_m](C)[ps].m(C)[ps]. | 49 |
| | MPK | [ps].m(A)p.m(U)p.m(A)p.[fl2r] | | m(G)p.m(A)p.m(C)p.[rc16](A) | |
| | | (C)p.m(C)p.m(G)p.m(A)p.m(G) | | p.[fl2r](U)p.[fl2r](U)p.[fl2r](C) | |
| | | p.m(G)p.m(A)p.m(A)p.[fl2r](U) | | p.m(C)p.m(U)p.m(C)p.m(G)p. | |
| | | p.m(G)p.[fl2r](U)p.m(C)p.m(G) | | m(G)p.m(U)p.m(A)p.m(U)[ps]. | |
| | | p.m(G)[ps].m(U)[ps].m(U) | | m(U)[ps].m(A) | |

Base5vp_una2moe HO 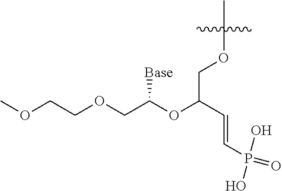
5am6_p_m 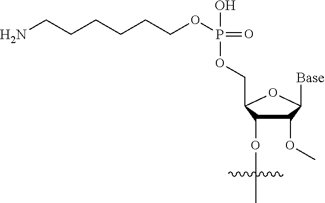
rc 16

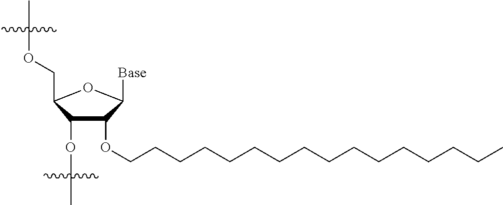

In Table 40, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

### Results

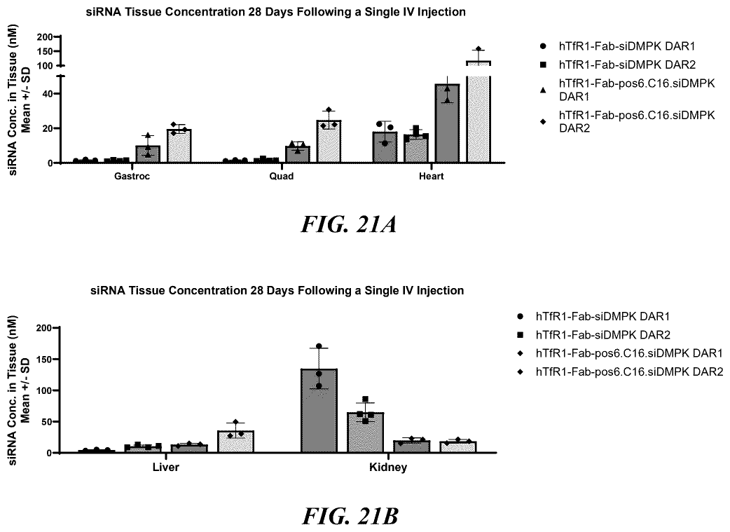

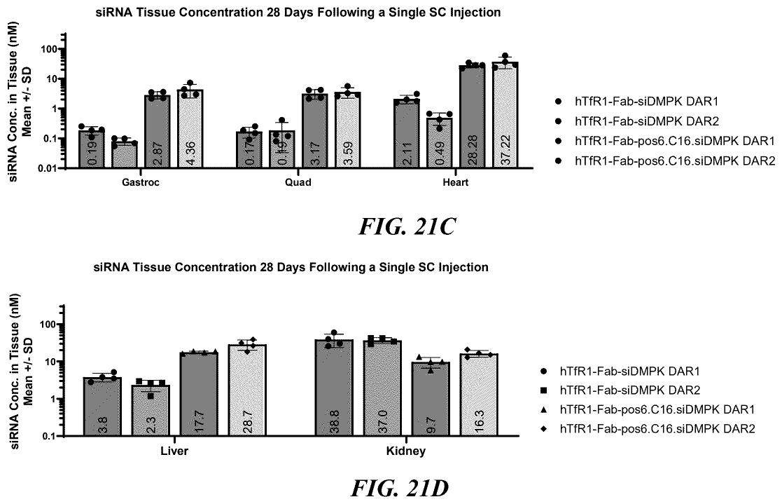

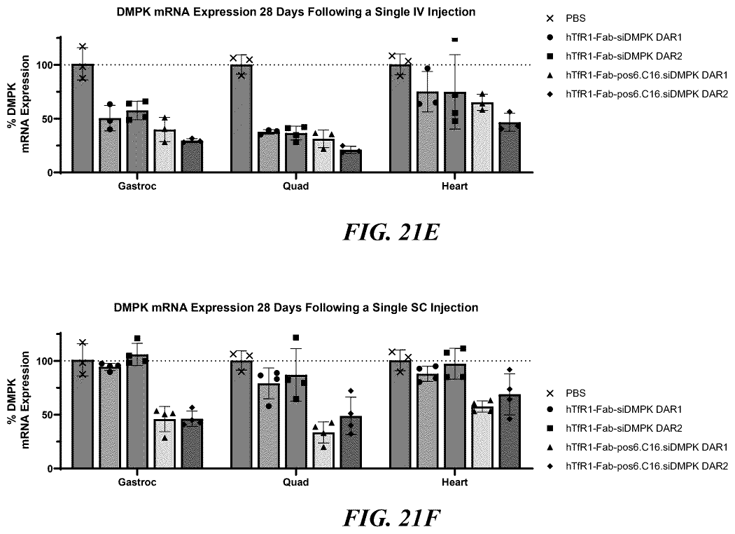

As shown in FIGS. 21A-21F, DMPK siRNA tissue concentrations and DMPK mRNA expression levels were assessed with DAR1/2 lipid-FabOC (Fab conjugated with oligonucleotide (siRNA) and lipid moiety) with the C16 hydrocarbon conjugated at position 6 passenger strand at day 28 in the gastroc (gastroc), tibialis anterior (TA), heart, and liver tissues obtained from mice following a single intravenous or subcutaneous injection of a DAR1/2 control FabOC (hTfR1-Fab-siDMPK DAR1/2) or DAR1/2 lipid FabOC with the C16 hydrocarbon conjugated at position 6 of the passenger strand (hTfR1-Fab-pos6.C16.siDMPK DAR1/2) of the siRNA a dose of 0.6 mg/kg siRNA for DAR1 or 1.2 mg/kg siRNA dose for DAR2 at Day 0.

At day 28 siRNA tissue concentrations were quantified in tissue obtained from humanized mice that were administered a single intravenous injection of DAR1 or DAR2 lipid-FabOCs with C16 conjugated at position 6 of the passenger strand. All lipid-FabOCs were able to deliver higher amount of siRNA to all muscle tissues than the non-lipid FabOCs (FIG. 21A). For instance, the DAR2 lipid FabOCs was able to deliver siRNA to gastroc muscle tissue at the concentration 19.5 nM while the FabOCs only delivered siRNA to muscle tissue at the concentration of 1.3 nM. The DAR2 lipid FabOCs delivered a 15-fold increase over the FabOCs in the gastroc muscle tissue. The increased in siRNA muscle tissue concentrations with lipid FabOCs were also measured in quad muscle tissue and heart.

However, the increases in siRNA tissue concentrations by lipid-FabOCs in the liver were less than the ones in muscle tissues (only a 4-fold increase over FabOCs) and no significant increases in siRNA tissue concentrations were observed in the kidney (FIG. 21B).

At day 28 siRNA tissue concentrations were quantified in tissue obtained from humanized mice that were administered a single subcutaneous injection of DAR1 or DAR2 lipid-FabOCs with C16 conjugated at position 6 of the passenger strand. All lipid-FabOCs were able to deliver higher amount of siRNA to all muscle tissues than the non-lipid FabOCs (FIG. 21C). For instance, the DAR2 lipid FabOCs showed the best activities and was able to deliver siRNA to gastroc muscle tissue at the concentration 37.2 nM while the FabOCs only delivered siRNA to muscle tissue at the concentration of 0.49 nM. The DAR2 lipid FabOCs delivered a 70-fold increase over the FabOCs in the heart muscle tissue. The increased in siRNA muscle tissue concentrations with lipid FabOCs were also measured in the quad muscles and gastroc muscles.

However, the increases in siRNA concentrations by lipid-FabOCs in the liver were less than the ones in muscle tissue (only 12-fold increase over FabOCs) and no significant increases in siRNA tissue concentrations were measured in the kidney (FIG. 21D).

At day 28 mRNA expression levels were quantified in tissue obtained from humanized mice that were administered a single intravenous injection of DAR1 or DAR2 lipid-FabOCs with C16 conjugated at position 6 of the passenger strand. Lipid-FabOCs and non-lipid FabOCs were able to decrease mRNA expression levels in all muscle tissues when administered intravenously (FIG. 21E). Lipid-FabOCs showed higher activities in decreasing target mRNA expression levels than FabOCs. Among the lipid-FabOCs, DAR2 lipid FabOCs had the highest activity in decreasing mRNA expression levels in all muscle tissues. For instance, DAR2 FabOCs decreased mRNA expression levels by 70% in gastro, 80% in quad and 50% in the heart.

At day 28 mRNA expression levels were quantified in tissue obtained from humanized mice that were administered a single subcutaneous injection of DAR1 or DAR2 lipid FabOCs with C16 hydrocarbon chain conjugated at position 6 of the passenger strand. Interestingly, only lipid FabOCs were able decrease mRNA expression levels in all muscle tissues when administered subcutaneously (FIG. 21F). The FabOCs did not have any significant effects in decreasing mRNA expression levels. Lipid FabOCs had higher activities in decreasing mRNA expression levels than FabOCs. For instance, DAR2 lipid FabOC was able to decrease mRNA expression levels in all muscle tissues. For instance, DAR2 FabOCs induced a decrease in mRNA expression levels by 50% in gastro and quad while DAR1 FabOCs induced a decrease in mRNA expression levels by 50% in the gastroc and by 65% in the quad.

Overall, lipid FabOCs administered intravenously or subcutaneously were able to deliver higher levels of siRNA to tissues and higher decreases in mRNA expression levels in muscle tissues compared to the non-lipid FabOCs.

### Example 14: Duration of Action of Human TfR1 Targeted Fab C22 siRNA Conjugates (FabOC) in Humanized TfR1 Mice

DMPK: A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The passenger strand was modified with 2′-O—C22 at position 3 nucleotide.

### Conjugate Synthesis and Characterization

The CD71 Fab-siRNA conjugates were made, purified and characterized as described in Examples 1-3. All conjugates were made through cysteine conjugation with a SMCC linker. Conjugates were characterized chromatographically as described in Table 41.

TABLE 41
HPLC Retention Time (RT) in Minutes

| | | RT, SAX | RT, SEC |
|---|---|---|---|
| Group(s) | Conjugate | Method-3 | Method-2 |
| 1 | hTfR1-Fab-siDMPK DAR1 | 3.744 | 15.631 |
| 2 | hTfR1-Fab-siDMPK DAR2 | 4.175 | 14.336 |
| 3-4 | hTfR1-Fab-pos3.C22.siDMPK DAR1 | 3.821 | 17.520 |
| 5-6 | hTfR1-Fab-pos3.C22.siDMPK DAR2 | 4.285 | 14.697 |

### In Vivo Study Design

The conjugates were assessed for their ability to mediate mRNA downregulation of myotonic dystrophy protein kinase (DMPK) in skeletal muscle in vivo in humanized TfR1 mice. Mice were dosed via intravenous (IV) injection or subcutaneous (SC) with PBS vehicle control or the indicated AOCs and at 2.14 mg/kg mAb protein dose, or 0.6 mg/kg for the DAR1 conjugates and 1.2 mg/kg for the DAR2 conjugates as shown in Table 42. After 28 days, gastrocnemius (gastroc), tibialis anterior (TA), liver, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 42
In vivo study design testing duration of action of DMPK siRNA Fab AOCs in
humanized TfR1 Mice

| | | siRNA | Fab | | | |
|---|---|---|---|---|---|---|
| | | dose | Dose | Doses | | Harvest |
| Group | Test Article | (mg/kg) | (mg/kg) | (#) | ROA | (Day) |
| 1 | hTfR1-Fab-siDMPK DAR1 | 0.6 | 2.14 | 1 | IV | 28 |
| 2 | hTfR1-Fab-siDMPK DAR2 | 1.2 | 2.14 | 1 | IV | 28 |
| 3 | hTfR1-Fab-pos3.C22.siDMPK DAR1 | 0.6 | 2.14 | 1 | IV | 28 |
| 4 | hTfR1-Fab-pos3.C22.siDMPK DAR1 | 0.6 | 2.14 | 1 | SC | 28 |
| 5 | hTfR1-Fab-pos3.C22.siDMPK DAR2 | 1.2 | 2.14 | 1 | IV | 28 |
| 6 | hTfR1-Fab-pos3.C22.siDMPK DAR2 | 1.2 | 2.14 | 1 | SC | 28 |
| 7 | PBS | N/A | N/A | 1 | IV | 28 |

* All animals used in experiments shown in Table 42 received a single dose.

TABLE 43
siRNA Sequences used in Example 14

| | | | SEQ | | SEQ |
|---|---|---|---|---|---|
| Compound | SiRNA | | ID | | ID |
| No. | Ref. | siRNA Guide Sequence | NO: | siRNA Passenger Sequence | NO: |
| 1 | siDMPK | [5vp_una2moe](U)[ps]. [f12r](A) | 12 | [5am6_p_m](C)[ps].m(C)[ps].m(G) | 48 |
| | | [ps].m(A)p.m(U)p.m(A)p.[fl2r](C) | | p.m(A)p.m(C)p.m(A)p.[fl2r](U)p. | |
| | | p.m(C)p.m(G)p.m(A)p.m(G)p.m( | | [fl2r](U)p.[fl2r](C)p.m(C)p.m(U)p. | |
| | | G)p.m(A)p.m(A)p.[fl2r](U)p.m(G) | | m(C)p.m(G)p.m(G)p.m(U)p.m(A) | |
| | | p.[fl2r](U)p.m(C)p.m(G)p.m(G) | | p.m(U)[ps].m(U)[ps].m(A) | |
| | | [ps].m(U)[ps].m(U) | | | |
| 2 | Pos3.C22 | [5vp_una2moe](U)[ps].[f12r](A) | 12 | [5am6_p_m](C)[ps].m(C)[ps].[rc2 | 81 |
| | .siDMPK | [ps].m(A)p.m(U)p.m(A)p.[fl2r](C) | | 2](G)p.m(A)p.m(C)p.m(A)p.[f12r] | |
| | | p.m(C)p.m(G)p.m(A)p.m(G)p.m | | (U)p.[f12r](U)p.[fl2r](C)p.m(C)p.m | |
| | | (G)p.m(A)p.m(A)p.[fl2r](U)p.m(G) | | (U)p.m(C)p.m(G)p.m(G)p.m(U)p.m | |
| | | p.[fl2r](U)p.m(C)p.m(G)p.m(G) | | (A)p.m(U)[ps].m(U)[ps].m(A) | |
| | | [ps].m(U)[ps].m(U) | | | |

5vp_una2moe 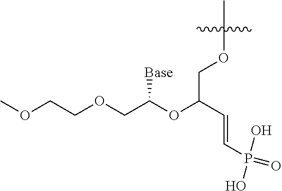
5am6_p_m 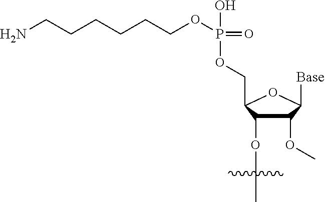
rc22

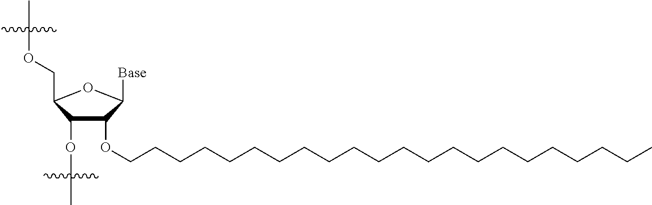

In Table 43, m indicates the 2′-O-methyl (2′-OMe) ribose modification; [fl2r] indicates the 2′-deoxy-″-fluoro (2′-F) ribose modification; [ps] indicates phosphorothioate (PS) linkage; p indicates phosphodiester (PO) linkage.

### Results

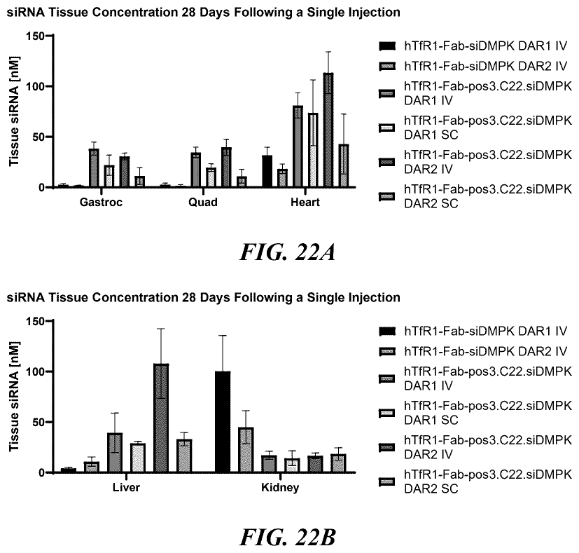

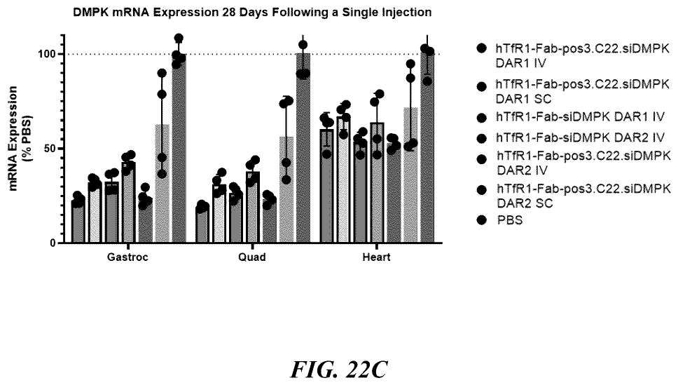

As shown in FIGS. 22A-22C, DMPK siRNA tissue concentrations and DMPK mRNA expression levels were assessed with DAR1 and DAR2 lipid FabOCs with the C22 hydrocarbon chain conjugated at position 3 passenger strand in gastroc, tibialis anterior (TA), heart, and liver obtained from mice following a single intravenous (IV) or subcutaneous (SC) injection of a DAR1 and DAR2 control FabOC (hTfR1-Fab-siDMPK DAR1 and DAR2) or the DAR1 and DAR2 lipid FabOC with the C22 hydrocarbon chain conjugated at position 3 of the passenger strand (hTfR1-Fab-pos3.C22.siDMPK DAR1 and DAR2) of the siRNA (2.14 mg/kg mAb protein dose, or 0.6 mg/kg for the DAR1 conjugates and 1.2 mg/kg for the DAR2 conjugates) at Day 0.

At day 28 siRNA Tissue concentrations were quantified in tissue obtained from humanized mice that were administered a single intravenous or subcutaneous injection of DAR1 or DAR2 lipid FabOCs with C22 hydrocarbon chain conjugated at position 3 of the passenger strand. All lipid-FabOCs were able to deliver higher amount of siRNA to all muscle tissues than the non-lipid FabOCs (FIG. 22A). Lipid FabOCs delivered higher levels of siRNA in cardiac tissue than the levels in skeletal muscles. The lipid FabOCs administered intravenously delivered higher siRNA tissue concentrations than the lipid FabOCs administered subcutaneously. For instance, the DAR1 lipid FabOCs administered intravenously delivered 38 nM siRNA concentration in gastroc while DAR1 lipid FabOCs delivered 22 nM when administered subcutaneously (FIG. 22A). In addition, lipid FabOCs delivered higher amount of siRNA than FabOCs in the liver, but FabOCs delivered higher amount of siRNA in the kidney (FIG. 22B).

At day 28, mRNA expression levels were quantified in tissue obtained from humanized mice that were administered a single intravenous or subcutaneous injection of DAR1 or DAR2 lipid FabOCs with C22 hydrocarbon chain conjugated at position 3 of the passenger strand of the siRNA. All lipid FabOCs administered intravenously were able to decrease mRNA expression levels in skeletal muscle tissue and cardiac tissue (FIG. 22C). Although the DAR1 lipid FabOCs administered subcutaneously had similar decreases in mRNA expression levels as the DAR1 FabOCs administered intravenously, the DAR2 lipid FabOCs administered subcutaneously had a much smaller decrease in mRNA expression levels compared to the DAR2 lipid FabOCs administered intravenously.

Overall, lipid-FabOCs with the C22 hydrocarbon chain conjugated at position 3 of the passenger strand administered subcutaneously or intravenously were able to deliver high amount of siRNA to tissues and decrease mRNA expression levels in skeletal muscle tissues.

### Example 15: Duration of Action of TfR1-Targeted Fab siRNA Conjugates with C16 on Fab or on a Linker in Wild Type Mice

DMPK: A 21-mer double-stranded siRNA with 19 bases of complementarity and 3′ dinucleotide overhangs was designed against mouse DMPK. The sequence (5′ to 3′) of the guide/antisense strand was UUAUUAUUUGUUCUUUGCCUU (SEQ ID NO: 4). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules s were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The passenger strand was modified with 2′-O—C22 at position 3 nucleotide.

### Conjugate Synthesis and Characterization

The CD71 Fab-siRNA conjugates were made, purified and characterized as described in Example 3. All conjugates were made through cysteine conjugation with a SMCC linker or with C16-linker as described. Conjugates were characterized chromatographically as described in Table 44.

TABLE 44
HPLC Retention Time (RT) in Minutes

| | | RT, SAX | RT, SEC |
|---|---|---|---|
| Group(s) | Conjugate | Method-5 | Method-2 |
| 1 | mTfR1-Fab-(vp)siDMPK DAR1 | 9.254 | 15.072 |
| 2 | mTfR1-Fab-(vp)pos6.C16.siDMPK DAR1 | 9.311 | 16.311 |
| 3 | mTfR1-(C16)Fab-(vp)siDMPK DAR1 | 9.138 | 19.039 |
| 4 | mTfR1-Fab-C16.linker-(vp)siDMPK DAR1 | 9.336 | 15.201 |

### In Vivo Study Design

The conjugates were assessed for their ability to mediate mRNA downregulation of myotonic dystrophy protein kinase (DMPK) in skeletal muscle in vivo in humanized TfR1 mice. Mice were dosed via intravenous (IV) injection or subcutaneous (SC) with PBS vehicle control or the indicated AOCs and at 2.14 mg/kg mAb protein dose, or 0.6 mg/kg for the DAR1 conjugates and 1.2 mg/kg for the DAR2 conjugates as shown in Table 45. After 28 days, gastrocnemius (gastroc), tibialis anterior (TA), liver, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 45
In vivo study design testing duration of action of DMPK siRNA Fab AOCs in
wild type mice

| | | | | siRNA | | Dose | |
|---|---|---|---|---|---|---|---|
| | | | | Dose | Doses | Volume | Harvest |
| Group | Test Article | N | DAR | (mg/kg) | (#) | (mL/kg) | (day) |
| 1 | mTfR1-Fab-(vp)siDMPK DAR1 | 4 | 1 | 1 | 1 | 5.0 | 28 |
| 2 | mTfR1-Fab-(vp)pos6.C16.siDMPK DAR1 | 4 | 1 | 1 | 1 | 5.0 | 28 |
| 3 | mTfR1-(C16)Fab-(vp)siDMPK DAR1 | 4 | 1 | 1 | 1 | 5.0 | 28 |
| 4 | mTfR1-Fab-C16.linker-(vp)siDMPK | 4 | 1 | 1 | 1 | 5.0 | 28 |
| | DAR1 | | | | | | |
| 5 | PBS | 4 | N/A | N/A | N/A | 5.0 | 28 |

* All animals used in experiments shown in Table 45 were 0.035 kg in body weights, and were dosed with single dose by IV administration

TABLE 46
siRNA Sequences used in Example 15

| | | | SEQ | | SEQ |
|---|---|---|---|---|---|
| Compound | siRNA | | ID | | ID |
| No. | Ref. | siRNA Guide Sequence | NO: | siRNA Passenger Sequence | NO: |
| 1 | (vp)siD | [5vp_una2moe](U)[ps].[fl2r](A) | 12 | [5am6_p_m](C)[ps].m(C)[ps].m | 48 |
| | MPK | [ps].m(A)p.m(U)p.m(A)p.[fl2r] | | (G)p.m(A)p.m(C)p.m(A)p.[fl2r] | |
| | | (C)p.m(C)p.m(G)p.m(A)p.m(G)p.m | | (U)p.[fl2r](U)p.[fl2r](C)p.m(C)p. | |
| | | (G)p.m(A)p.m(A)p.[fl2r](U)p.m | | m(U)p.m(C)p.m(G)p.m(G)p.m | |
| | | (G)p.[fl2r](U)p.m(C)p.m(G)p.m | | (U)p.m(A)p.m(U)[ps].m(U)[ps]. | |
| | | (G)[ps].m(U)[ps].m(U) | | m(A) | |
| 2 | (vp)pos6. | [5vp_una2moe](U)[ps].[f12r](A) | 12 | [5am6_p_m](C)[ps].m(C)[ps].m | 49 |
| | C16.siD | [ps].m(A)p.m(U)p.m(A)p. [fl2r] | | (G)p.m(A)p.m(C)p.[rc16](A)p.[f | |
| | MPK | (C)p.m(C)p.m(G)p.m(A)p.m(G)p.m | | 12r](U)p.[f12r](U)p.[f12r](C)p.m | |
| | | (G)p.m(A)p.m(A)p.[fl2r](U)p. | | (C)p.m(U)p.m(C)p.m(G)p.m(G)p.m | |
| | | m(G)p.[fl2r](U)p.m(C)p.m(G)p. | | (U)p.m(A)p.m(U)[ps].m(U)[p | |
| | | m(G)[ps].m(U)[ps].m(U) | | s].m(A) | |

5vp_una2moe 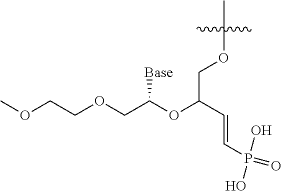
5am6_p_m 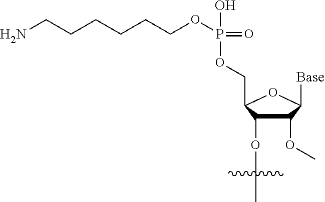
Rc16

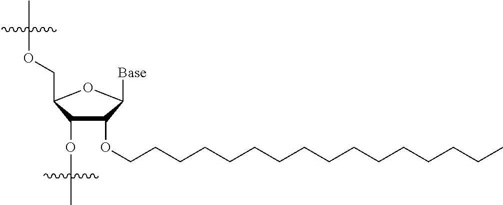

In Table 46, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

### Results

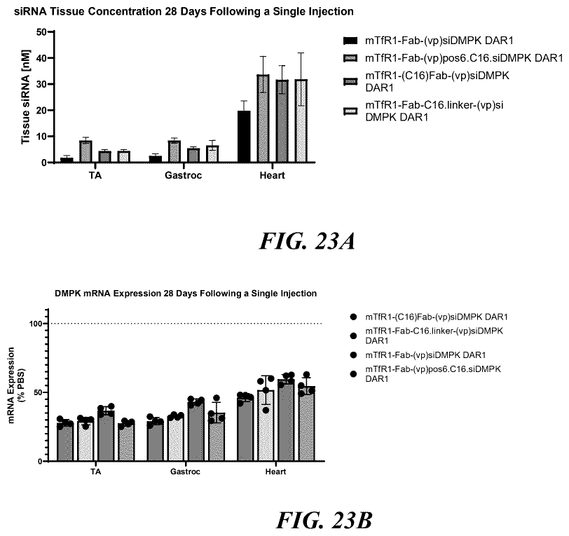

As shown in FIGS. 23A-23B, DMPK siRNA tissue concentrations and DMPK mRNA expression levels were assessed with DAR1 lipid-FabOC with the C16 hydrocarbon chain conjugated at position 6 passenger strand or with the lipid conjugated to the linker of the FabOC at day 28 in the gastroc, tibialis anterior (TA), and heart obtained from mice following a single intravenous (IV) injection of the DAR1 FabOC (mTfR1-Fab-(vp)siDMPK DAR1), the DAR1 lipid FabOC with the C16 hydrocarbon chain conjugated at position 6 nucleotide of the passenger strand (mTfR1-Fab-(vp)pos6.C16.siDMPK DAR1), the DAR1 FabOC with the C16 hydrocarbon chain conjugated to Fab (mTfR1-(C16)Fab-(vp)siDMPK DAR1), the DAR1 FabOC with the C16 hydrocarbon chain conjugated to the linker (mTfR1-Fab-C16.linker-(vp)siDMPK DAR1) of the VP modified siRNA at Day 0.

At day 28 DMPK siRNA tissue concentrations were quantified in tissue obtained from wild type mice that were administered a single intravenous injection of DAR1 lipid FabOCs with C16 hydrocarbon chain conjugated at position 6 nucleotide of the passenger strand or DAR1 lipid FabOCs with C16 hydrocarbon chain conjugated to the linker linking the Fab to the siRNA.

As shown in FIG. 23A, all lipid FabOCs were able to deliver higher amount of siRNA to all muscle tissues than the non-lipid FabOCs. The lipid FabOCs with the lipid conjugated to the linker (mTfR1-Fab-C16.linker-(vp)siDMPK DAR1) or to the Fab (mTfR1-(C16)Fab-(vp)siDMPK DAR1) delivered similar amounts of siRNA in the TA and gastroc muscle tissues. However, these 2 lipid FabOCs were delivered less than the DAR1 lipid FabOCs with C16 hydrocarbon chain conjugated at position 6 nucleotide of the passenger strand (mTfR1-Fab-(vp)pos6.C16.siDMPK DAR1).

As shown in FIG. 23B, all lipid FabOCs were able to mRNA expression levels than FabOCs in all muscle tissues. The 3 lipid FabOCs were able to induce similar mRNA expression levels in skeletal muscles (TA and Gastroc).

Overall, the lipid FabOCs with C16 hydrocarbon chain conjugated to the linker has similar biological activities as the lipid FabOCs with C16 hydrocarbon chain conjugated to the passenger strand of the siRNA.

### Example 16: Duration of Action of TfR1-Targeted Fab siRNA Conjugates with Lipid Linkers in Wild Type Mice

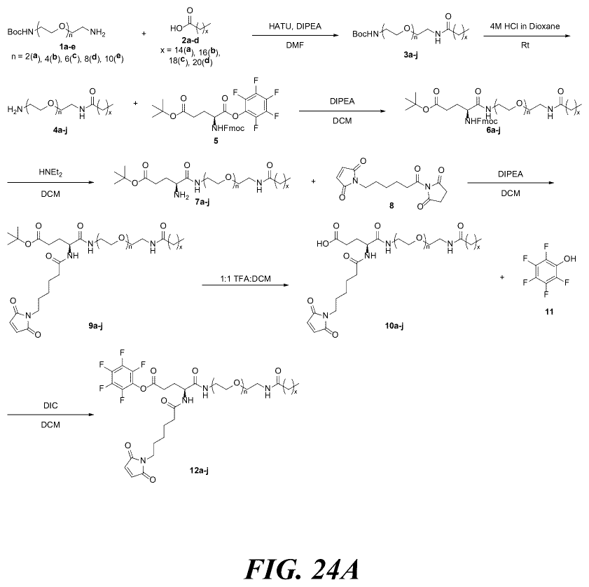

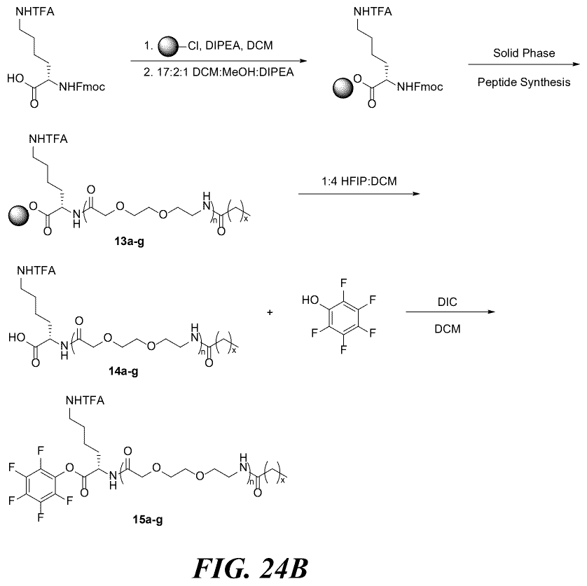

A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The synthesis pathways of the 12a-j compounds and 15a-j compounds are shown in FIGS. 24A-24B, respectively.

### LIPID LINKER SYNTHESIS AND CHARACTERIZATION

### Synthesis of Linkers 12a-i

### Synthesis of Compound 3a

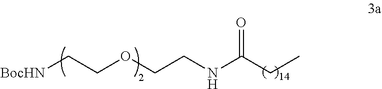

To a solution of compound 1a (277 mg, 1.115 mmol, 1.1 equiv.), compound 2a (260 mg, 1.014 mmol, 1.0 equiv.), and HATU (404.8 mg, 1.065 mmol, 1.05 equiv.) in anhydrous DMF (5 mL) was added DIPEA (0.529 mL, 3.042 mmol, 3.0 equiv.). The resulting solution was stirred at RT until full conversion by LC-MS. The reaction mixture was then diluted with water (25 mL). The product was extracted with ethyl acetate (5×15 mL) and then washed with water (2×20 mL), 10% LiCl (2×20 mL) and brine (1×20 mL). The combined organic phase was dried over anhydrous Na₂SO₄, filtered, and concentrated. The residue was purified by Combiflash® using silica gel as the stationary phase with a gradient of 0-80% ethyl acetate in heptane, in which the product eluted at 80% B. The product was concentrated under vacuum to provide a colorless solid. LC-MS: calculated [M+H]+ 487.41, observed 487.52.

### Synthesis of Compound 3b

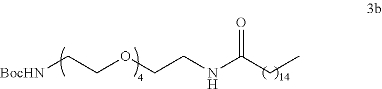

To a solution of compound 1b (375.2 mg, 1.115 mmol, 1.1 equiv.), compound 2a (260 mg, 1.014 mmol, 1.0 equiv.), and HATU (404.8 mg, 1.065 mmol, 1.05 equiv.) in anhydrous DMF (5 mL) was added DIPEA (0.529 mL, 3.042 mmol, 3.0 equiv.). The resulting solution was stirred at RT until full conversion by LC-MS. The reaction mixture was then diluted with water (25 mL). The product was extracted with ethyl acetate (5×15 mL) and then washed with water (2×20 mL), 10% LiCl (2×20 mL) and brine (1×20 mL). The combined organic phase was dried over anhydrous Na₂SO₄, filtered, and concentrated. The residue was purified by Combiflash® using silica gel as the stationary phase with a gradient of 0-100% ethyl acetate in heptane, in which the product eluted at 80% B. The product was concentrated under vacuum to provide a colorless solid. LC-MS: calculated [M+H]+ 575.46, observed 575.46.

### Synthesis of Compound 3c

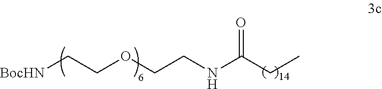

To a solution of compound 1c (200.00 mg, 0.780 mmol, 1.0 equiv.), compound 2a (331.00 mg, 0.780 mmol, 1.0 equiv.), and HATU (297.00 mg, 0.780 mmol, 1.0 equiv.) in anhydrous DMF (4 mL) was added DIPEA (0.408 mL, 2.340 mmol, 3.0 equiv.). The resulting solution was stirred at RT until full conversion by LC-MS. The reaction mixture was then diluted with water (25 mL). The product was extracted with ethyl acetate (5×15 mL) and then washed with water (2×20 mL), 10% LiCl (2×20 mL) and brine (1×20 mL). The combined organic phase was dried over anhydrous Na₂SO₄, filtered, and concentrated. The residue was purified by Combiflash® using silica gel as the stationary phase with a gradient of 0-20% methanol in dichloromethane, in which the product eluted at 20% B. The product was concentrated under vacuum to provide a colorless solid. LC-MS: calculated [M+H]+ 663.52, observed 663.63.

### Synthesis of Compound 3d

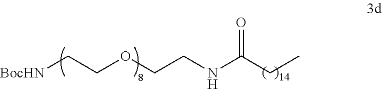

To a solution of compound 1d (571.76 mg, 1.115 mmol, 1.1 equiv.), compound 2a (260 mg, 1.014 mmol, 1.0 equiv.), and HATU (404.8 mg, 1.065 mmol, 1.05 equiv.) in anhydrous DMF (5 mL) was added DIPEA (0.529 mL, 3.042 mmol, 3.0 equiv.). The resulting solution was stirred at RT until full conversion by LC-MS. The reaction mixture was then diluted with water (25 mL). The product was extracted with ethyl acetate (5×15 mL) and then washed with water (2×20 mL), 10% LiCl (2×20 mL) and brine (1×20 mL). The combined organic phase was dried over anhydrous Na₂SO₄, filtered, and concentrated. The residue was purified by Combiflash® using silica gel as the stationary phase with a gradient of 0-20% methanol in dichloromethane, in which the product eluted at 20% B. The product was concentrated under vacuum to provide a colorless solid. LC-MS: calculated [M+H]+ 751.57, observed 751.59.

### Synthesis of Compound 3e

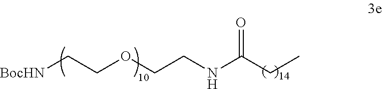

To a solution of compound 1e (60.00 mg, 0.234 mmol, 1.0 equiv.), compound 2a (141.00 mg, 0.234 mmol, 1.0 equiv.), and HATU (89.00 mg, 0.234 mmol, 1.0 equiv.) in anhydrous DMF (1.2 mL) was added DIPEA (0.122 mL, 0.702 mmol, 3.0 equiv.). The resulting solution was stirred at RT until full conversion by LC-MS. The reaction mixture was then diluted with water (25 mL). The product was extracted with ethyl acetate (5×15 mL) and then washed with water (2×20 mL), 10% LiCl (2×20 mL) and brine (1×20 mL). The combined organic phase was dried over anhydrous Na₂SO₄, filtered, and concentrated. The residue was purified by Combiflash® using silica gel as the stationary phase with a gradient of 0-80% 3:1 ethyl acetate:ethanol in heptane, in which the product eluted at 80% B. The product was concentrated under vacuum to provide a white solid. LC-MS: calculated [M+H]+ 839.62, observed 839.64.

### Synthesis of Compound 3f

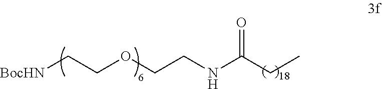

To a solution of compound 1c (346.42 mg, 0.816 mmol, 1.0 equiv.), compound 2c (255 mg, 0.816 mmol, 1.0 equiv.), and HATU (341.29 mg, 0.898 mmol, 1.1 equiv.) in anhydrous DMF (4 mL) was added DIPEA (0.426 mL, 2.448 mmol, 3.0 equiv.). The resulting solution was stirred at RT until full conversion by LC-MS. The reaction mixture was then diluted with water (25 mL). The product was extracted with ethyl acetate (5×15 mL) and then washed with water (2×20 mL), 10% LiCl (2×20 mL) and brine (1×20 mL). The combined organic phase was dried over anhydrous Na₂SO₄, filtered, and concentrated. The residue was purified by Combiflash® using silica gel as the stationary phase with a gradient of 0-90% 3:1 ethyl acetate:ethanol in heptane, in which the product eluted at 90% B. The product was concentrated under vacuum to provide a colorless solid. LC-MS: calculated [M+H]+ 719.58, observed 719.59.

### Synthesis of Compound 3g

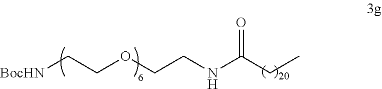

To a solution of compound 1c (317.84 mg, 0.749 mmol, 1.0 equiv.), compound 2d (255 mg, 0.749 mmol, 1.0 equiv.), and HATU (313.14 mg, 0.824 mmol, 1.1 equiv.) in anhydrous DMF (4 mL) was added DIPEA (0.391 mL, 2.246 mmol, 3.0 equiv.). The resulting solution was stirred at RT until full conversion by LC-MS. The reaction mixture was then diluted with water (25 mL). The product was extracted with ethyl acetate (5×15 mL) and then washed with water (2×20 mL), 10% LiCl (2×20 mL) and brine (1×20 mL). The combined organic phase was dried over anhydrous Na₂SO₄, filtered, and concentrated. The residue was purified by Combiflash® using silica gel as the stationary phase with a gradient of 0-90% 3:1 ethyl acetate:ethanol in heptane, in which the product eluted at 90% B. The product was concentrated under vacuum to provide a colorless solid. LC-MS: calculated [M+H]+ 747.61, observed 747.61.

### Synthesis of Compound 3h

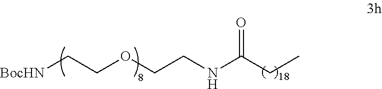

To a solution of compound 1d (229.66 mg, 0.448 mmol, 1.0 equiv.), compound 2c (140 mg, 0.448 mmol, 1.0 equiv.), and HATU (178.86 mg, 0.470 mmol, 1.05 equiv.) in anhydrous DMF (2.25 mL) was added DIPEA (0.234 mL, 1.344 mmol, 3.0 equiv.). The resulting solution was stirred at RT until full conversion by LC-MS. The reaction mixture was then diluted with water (25 mL). The product was extracted with ethyl acetate (5×15 mL) and then washed with water (2×20 mL), 10% LiCl (2×20 mL) and brine (1×20 mL). The combined organic phase was dried over anhydrous Na₂SO₄, filtered, and concentrated. The residue was purified by Combiflash® using silica gel as the stationary phase with a gradient of 0-90% 3:1 ethyl acetate:ethanol in heptane, in which the product eluted at 90% B. The product was concentrated under vacuum to provide a colorless solid. LC-MS: calculated [M+H]+ 807.63, observed 807.64.

### Synthesis of Compound 3i

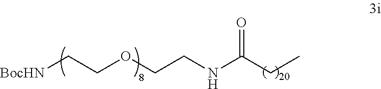

To a solution of compound 1d (229.66 mg, 0.448 mmol, 1.0 equiv.), compound 2d (140 mg, 0.448 mmol, 1.0 equiv.), and HATU (178.86 mg, 0.470 mmol, 1.05 equiv.) in anhydrous DMF (2.25 mL) was added DIPEA (0.234 mL, 1.344 mmol, 3.0 equiv.). The resulting solution was stirred at RT until full conversion by LC-MS. The reaction mixture was then diluted with water (25 mL). The product was extracted with ethyl acetate (5×15 mL) and then washed with water (2×20 mL), 10% LiCl (2×20 mL) and brine (1×20 mL). The combined organic phase was dried over anhydrous Na₂SO₄, filtered, and concentrated. The residue was purified by Combiflash® using silica gel as the stationary phase with a gradient of 0-90% 3:1 ethyl acetate ethanol in heptane, in which the product eluted at 90% B. The product was concentrated under vacuum to provide a colorless solid. LC-MS: calculated [M+H]+ 835.66, observed 835.67.

### Synthesis of Compound 3j

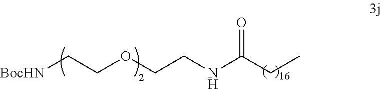

To a solution of compound 1a (249.00 mg, 1.002 mmol, 1.0 equiv.), compound 2b (285.00 mg, 1.002 mmol, 1.0 equiv.), and HATU (381.00 mg, 1.002 mmol, 1.0 equiv.) in anhydrous DMF (5 mL) was added DIPEA (0.524 mL, 3.005 mmol, 3.0 equiv.). The resulting solution was stirred at RT until full conversion by LC-MS. The reaction mixture was then diluted with water (25 mL). The product was extracted with ethyl acetate (5×15 mL) and then washed with water (2×20 mL), 10% LiCl (2×20 mL) and brine (1×20 mL). The combined organic phase was dried over anhydrous Na₂SO₄, filtered, and concentrated. The residue was purified by Combiflash® using silica gel as the stationary phase with a gradient of 0-80% 3:1 ethyl acetate in heptane, in which the product eluted at 80% B. The product was concentrated under vacuum to provide a colorless solid. LC-MS: calculated [M+H]+ 515.44, observed 515.55.

### Synthesis of Compound 4a

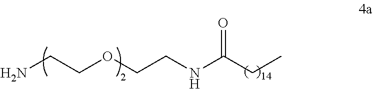

To compound 3a (472.00 mg, 1.000 mmol) was added 4 M HCl/Dioxane (5 mL) at RT. The reaction was stirred overnight until full conversion was confirmed via LC-MS. The reaction mixture was concentrated under vacuum to provide a white solid. LC-MS: calculated [M+H]+ 387.36, observed 387.46.

### Synthesis of Compound 4b

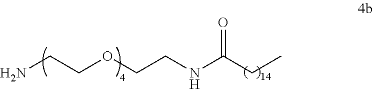

To compound 3b (640.2 mg, 1.000 mmol) was added 4 M HCl/Dioxane (5 mL) at RT. The reaction was stirred overnight until full conversion was confirmed via LC-MS. The reaction mixture was concentrated under vacuum to provide a white solid. LC-MS: calculated [M+H]+ 475.41, observed 475.42.

### Synthesis of Compound 4c

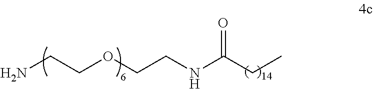

To compound 3c (451.00 mg, 0.680 mmol) was added 4 M HCl/Dioxane (3.4 mL) at RT. The reaction was stirred overnight until full conversion was confirmed via LC-MS. The reaction mixture was concentrated under vacuum to provide a white solid. LC-MS: calculated [M+H]+ 563.46, observed 563.51.

### Synthesis of Compound 4d

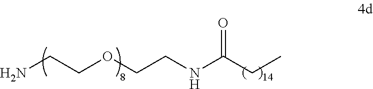

To compound 3d (630.70 mg, 0.840 mmol) was added 4 M HCl/Dioxane (5 mL) at RT. The reaction was stirred overnight until full conversion was confirmed via LC-MS. The reaction mixture was concentrated under vacuum to provide a clear oil. LC-MS: calculated [M+H]+ 651.52, observed 651.56.

### Synthesis of Compound 4e

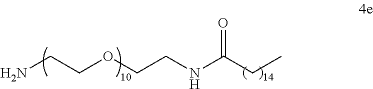

To compound 3e (173 mg, 0.206 mmol) was added 4 M HCl/Dioxane (1 mL) at RT. The reaction was stirred overnight until full conversion was confirmed via LC-MS. The reaction mixture was concentrated under vacuum to provide a clear oil. LC-MS: calculated [M+H]+ 739.57 observed 739.60.

### Synthesis of Compound 4f

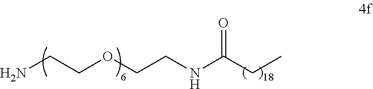

To compound 3f (624.20 mg, 0.868 mmol) was added 4 M HCl/Dioxane (5 mL) at RT. The reaction was stirred overnight until full conversion was confirmed via LC-MS. The reaction mixture was concentrated under vacuum to provide a clear oil. LC-MS: calculated [M+H]+ 619.53 observed 619.55.

### Synthesis of Compound 4g

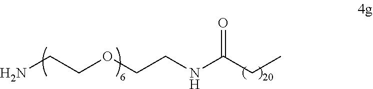

To compound 3g (541.2 mg, 0.724 mmol) was added 4 M HCl/Dioxane (2 mL) at RT. The reaction was stirred overnight until full conversion was confirmed via LC-MS. The reaction mixture was concentrated under vacuum to provide a white solid. LC-MS: calculated [M+H]+ 647.56 observed 647.57.

### Synthesis of Compound 4h

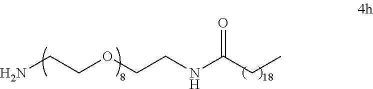

To compound 3h (228.6 mg, 0.307 mmol) was added 4 M HCl/Dioxane (4 mL) at RT. The reaction was stirred overnight until full conversion was confirmed via LC-MS. The reaction mixture was concentrated under vacuum to provide a white solid. LC-MS: calculated [M+H]+ 707.58 observed 707.60.

### Synthesis of Compound 4i

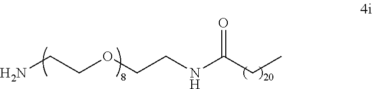

To compound 3i (235.00 mg, 0.305 mmol) was added 4 M HCl/Dioxane (4 mL) at RT. The reaction was stirred overnight until full conversion was confirmed via LC-MS. The reaction mixture was concentrated under vacuum to provide a white solid. LC-MS: calculated [M+H]+ 735.61 observed 735.62.

### Synthesis of Compound 4j

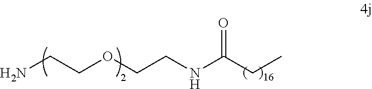

To compound 3j (260.00 mg, 0.505 mmol) was added 4 M HCl/Dioxane (2.5 mL) at RT. The reaction was stirred overnight until full conversion was confirmed via LC-MS. The reaction mixture was concentrated under vacuum to provide a white solid. LC-MS: calculated [M+H]+ 415.39 observed 415.50.

### Synthesis of Compound 6a

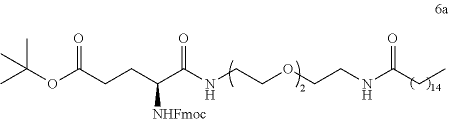

To a solution of compound 4a (454.70 mg, 1.075 mmol, 1.0 equiv.) and compound 5 (635.31 mg, 1.075 mmol, 1.0 equiv.) in dichloromethane (10 mL) was added DIPEA (1.869 mL, 10.747 mmol, 10 equiv.) dropwise. The resulting solution was stirred at RT until full conversion by LC-MS. The reaction mixture was then concentrated under vacuum. The residue was purified by Combiflash® using silica gel as the stationary phase with a gradient of 0-90% ethyl acetate in dichloromethane, in which the product eluted at 85% B. The product was concentrated under vacuum to provide an off-white solid. LC-MS: calculated [M+H]+ 794.53, observed 794.66.

### Synthesis of Compound 6b

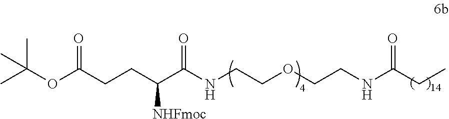

To a solution of compound 4b (512.00 mg, 1.002 mmol, 1.0 equiv.) and compound 5 (592.07 mg, 1.002 mmol, 1.0 equiv.) in dichloromethane (10 mL) was added DIPEA (1.741 mL, 10.016 mmol, 10 equiv.) dropwise. The resulting solution was stirred at RT until full conversion by LC-MS. The reaction mixture was then concentrated under vacuum. The residue was purified by Combiflash® using silica gel as the stationary phase with a gradient of 0-20% methanol in dichloromethane, in which the product eluted at 20% B. The product was concentrated under vacuum to provide an off-white solid. LC-MS: calculated [M+H]+ 882.58, observed 882.61.

### Synthesis of Compound 6c

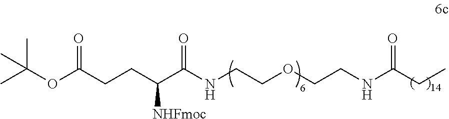

To a solution of compound 4c (363.00 mg, 0.644 mmol, 1.0 equiv.) and compound 5 (381 mg, 0.644 mmol, 1.0 equiv.) in dichloromethane (6.4 mL) was added DIPEA (1.122 mL, 6.442 mmol, 10 equiv.) dropwise. The resulting solution was stirred at RT until full conversion by LC-MS. The reaction mixture was then concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum and provided a white solid. LC-MS: calculated [M+H]+ 970.64, observed 970.66.

### Synthesis of Compound 6d

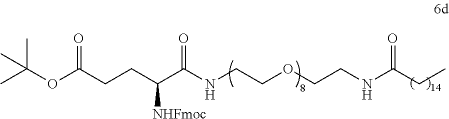

To a solution of compound 4d (577.20 mg, 0.840 mmol, 1.0 equiv.) and compound 5 (496.36 mg, 0.840 mmol, 1.0 equiv.) in dichloromethane (4.2 mL) was added DIPEA (1.460 mL, 8.297 mmol, 10 equiv.) dropwise. The resulting solution was stirred at RT until full conversion by LC-MS. The reaction mixture was then concentrated under vacuum. The residue was purified by Combiflash® using silica gel as the stationary phase with a gradient of 0-3% methanol in dichloromethane, in which the product eluted at 3% B. The product was concentrated under vacuum and provided a white foamy solid. LC-MS: calculated [M+H]+ 1058.41, observed 1058.70.

### Synthesis of Compound 6e

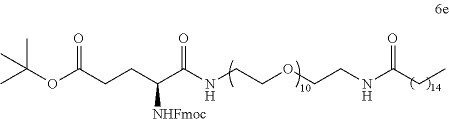

To a solution of compound 4e (154.00 mg, 0.208 mmol, 1.0 equiv.) and compound 5 (123.00 mg, 0.208 mmol, 1.0 equiv.) in dichloromethane (2.1 mL) was added DIPEA (0.363 mL, 2.084 mmol, 10 equiv.) dropwise. The resulting solution was stirred at RT until full conversion by LC-MS. The reaction mixture was then concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum and provided a white foamy solid. LC-MS: calculated [M+Na]+1168.72, observed 1168.73.

### Synthesis of Compound 6f

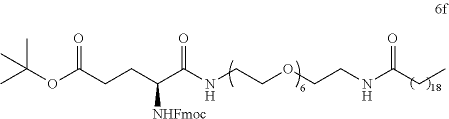

To a solution of compound 4f (575.26 mg, 0.804 mmol, 1.0 equiv.) and compound 5 (475.37 mg, 0.804 mmol, 1.0 equiv.) in dichloromethane (4.1 mL) was added DIPEA (1.398 mL, 8.042 mmol, 10 equiv.) dropwise. The resulting solution was stirred at RT until full conversion by LC-MS. The reaction mixture was then concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum and provided a white foamy solid. LC-MS: calculated [M+H]+ 1026.70, observed 1026.68.

### Synthesis of Compound 6g

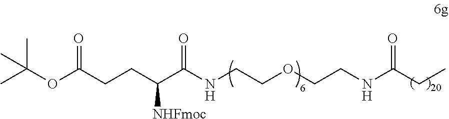

To a solution of compound 4g (535.7 mg, 0.721 mmol, 1.0 equiv.) and compound 5 (425.92 mg, 0.721 mmol, 1.0 equiv.) in dichloromethane (7.2 mL) was added DIPEA (1.253 mL, 7.205 mmol, 10 equiv.) dropwise. The resulting solution was stirred at RT until full conversion by LC-MS. The reaction mixture was then concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum and provided a white foamy solid. LC-MS: calculated [M+H]+ 1054.73, observed 1054.70.

### Synthesis of Compound 6h

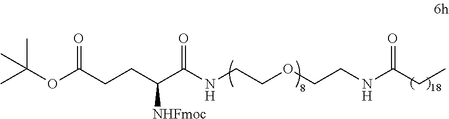

To a solution of compound 4h (228.60 mg, 0.816 mmol, 1.0 equiv.) and com8 pound 5 (482.36 mg, 0.816 mmol, 1.0 equiv.) in dichloromethane (4.1 mL) was added DIPEA (1.419 mL, 8.160 mmol, 10 equiv.) dropwise. The resulting solution was stirred at RT until full conversion by LC-MS. The reaction mixture was then concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum and provided a white foamy solid. LC-MS: calculated [M+H]+ 1114.75, observed 1114.75.

### Synthesis of Compound 6i

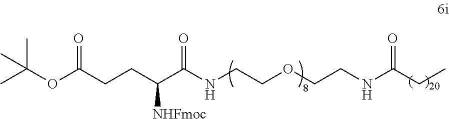

To a solution of compound 4i (235.00 mg, 0.816 mmol, 1.0 equiv.) and compound 5 (482.36 mg, 0.816 mmol, 1.0 equiv.) in dichloromethane (8.2 mL) was added DIPEA (1.419 mL, 8.160 mmol, 10 equiv.) dropwise. The resulting solution was stirred at RT until full conversion by LC-MS. The reaction mixture was then concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum and provided a white foamy solid. LC-MS: calculated [M+H]+ 1142.78, observed 1142.77.

### Synthesis of Compound 6j

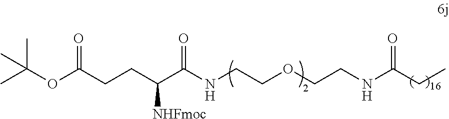

To a solution of compound 4j (31.0 mg, 0.074 mmol, 1.0 equiv.) and compound 5 (44.00 mg, 0.074 mmol, 1.0 equiv.) in dichloromethane (0.75 mL) was added DIPEA (0.129 mL, 0.740 mmol, 10 equiv.) dropwise. The resulting solution was stirred at RT until full conversion by LC-MS. The reaction mixture was then concentrated under vacuum. The residue was purified by Combiflash® using silica gel as the stationary phase with a gradient of 0-20% methanol in dichloromethane, in which the product eluted at 80% B. The product was concentrated under vacuum and provided a white foamy solid. LC-MS: calculated [M+Na]+844.55, observed 844.66.

### Synthesis of Compound 7a

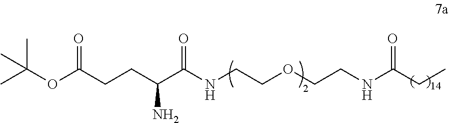

To compound 6a (849 mg, 1.07 mmol) was added solution of 25% diethylamine in dichloromethane (v/v) (5 mL). The resulting solution was stirred at RT until full conversion by LC-MS. The reaction mixture was concentrated under vacuum. The product was used directly without further purification. LC-MS: calculated [M+H]+ 572.46, observed 572.59.

### Synthesis of Compound 7b

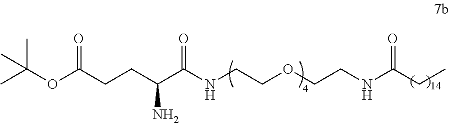

To compound 6b (650.90 mg, 0.820 mmol) was added solution of 25% diethylamine in dichloromethane (v/v) (2 mL). The resulting solution was stirred at RT until full conversion by LC-MS. The reaction mixture was concentrated under vacuum. The product was used directly without further purification. LC-MS: calculated [M+H]+ 660.52, observed 660.56.

### Synthesis of Compound 7c

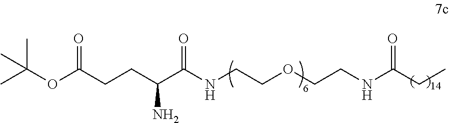

To compound 6c (500.00 mg, 0.515 mmol) was added solution of 25% diethylamine in dichloromethane (v/v) (5.2 mL). The resulting solution was stirred at RT until full conversion by LC-MS. The reaction mixture was concentrated under vacuum. The product was used directly without further purification. LC-MS: calculated [M+H]+ 748.57, observed 748.60.

### Synthesis of Compound 7d

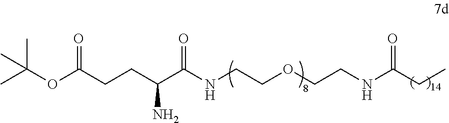

To compound 6d (725.00 mg, 0.685 mmol) was added solution of 25% diethylamine in dichloromethane (v/v) (4.0 mL). The resulting solution was stirred at RT until full conversion by LC-MS. The reaction mixture was concentrated under vacuum. The product was used directly without further purification. LC-MS: calculated [M+H]+ 836.62, observed 836.65.

### Synthesis of Compound 7e

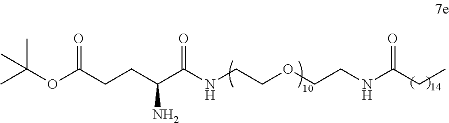

To compound 6e (177.00 mg, 0.154 mmol) was added solution of 25% diethylamine in dichloromethane (v/v) (1.5 mL). The resulting solution was stirred at RT until full conversion by LC-MS. The reaction mixture was concentrated under vacuum. The product was used directly without further purification. LC-MS: calculated [M+H]+ 924.67, observed 924.70.

### Synthesis of Compound 7f

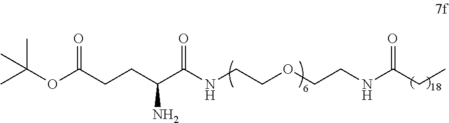

To compound 6f (499.00 mg, 0.486 mmol) was added solution of 25% diethylamine in dichloromethane (v/v) (2.0 mL). The resulting solution was stirred at RT until full conversion by LC-MS. The reaction mixture was concentrated under vacuum. The product was used directly without further purification. LC-MS: calculated [M+H]+ 804.63, observed 804.63.

### Synthesis of Compound 7g

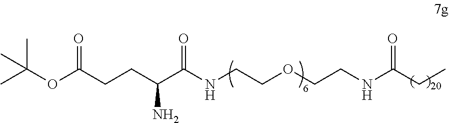

To compound 6g (542.40 mg, 0.514 mmol) was added solution of 25% diethylamine in dichloromethane (v/v) (2.0 mL). The resulting solution was stirred at RT until full conversion by LC-MS. The reaction mixture was concentrated under vacuum. The product was used directly without further purification. LC-MS: calculated [M+H]+ 832.66, observed 832.65.

### Synthesis of Compound 7h

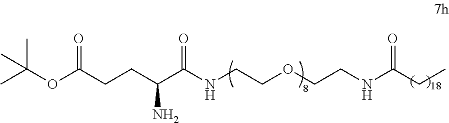

To compound 6h (174.00 mg, 0.156 mmol) was added solution of 25% diethylamine in dichloromethane (v/v) (2.0 mL). The resulting solution was stirred at RT until full conversion by LC-MS. The reaction mixture was concentrated under vacuum. The product was used directly without further purification. LC-MS: calculated [M+H]+ 892.68, observed 892.68.

### Synthesis of Compound 7i

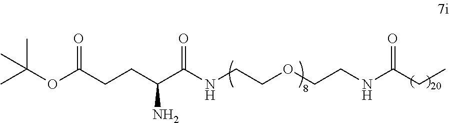

To compound 6i (304.60 mg, 0.267 mmol) was added solution of 25% diethylamine in dichloromethane (v/v) (2.0 mL). The resulting solution was stirred at RT until full conversion by LC-MS. The reaction mixture was concentrated under vacuum. The product was used directly without further purification. LC-MS: calculated [M+H]+ 920.72, observed 920.71.

### Synthesis of Compound 7j

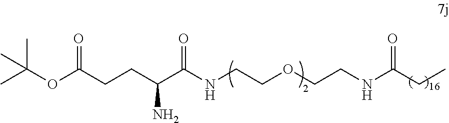

To compound 6j (50.00 mg, 0.061 mmol) was added solution of 25% diethylamine in dichloromethane (v/v) (0.6 mL). The resulting solution was stirred at RT until full conversion by LC-MS. The reaction mixture was concentrated under vacuum. The product was used directly without further purification. LC-MS: calculated [M+H]+ 600.50, observed 600.54.

### Synthesis of Compound 9a

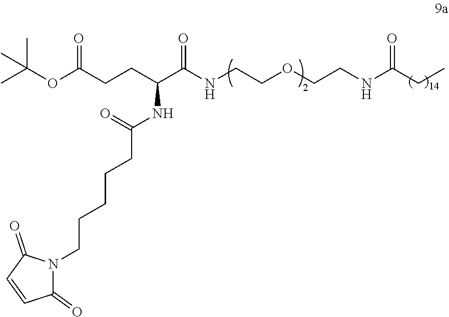

To a solution of compound 7a (610.89 mg, 1.069 mmol, 1.0 equiv.) and compound 8 (659.21 mg, 2.138 mmol, 3.0 equiv.) in dichloromethane (10.7 mL) was added DIPEA (0.558 mL, 3.207 mmol, 3.0 equiv.). The resulting solution was stirred at RT until full conversion by LC-MS. The reaction mixture was diluted with water (10 mL). The product was extracted with dichloromethane (3×20 mL). The combined organic phase washed with brine (1×20 mL), dried over anhydrous Na₂SO₄, filtered, and concentrated under vacuum. The residue was purified by Combiflash® using silica gel as the stationary phase with a gradient of 0-10% methanol in dichloromethane, in which the product eluted at 10% B. The product was concentrated under vacuum to provide a yellow solid. LC-MS: calculated [M+H]+ 765.54, observed 765.66.

### Synthesis of Compound 9b

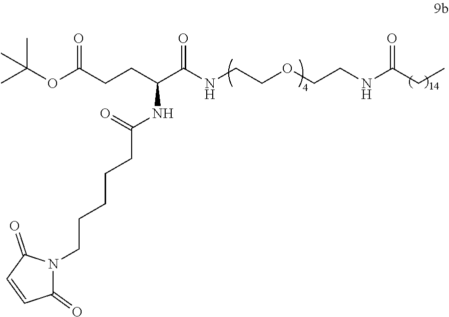

To a solution of compound 7b (540.95 mg, 0.820 mmol, 1.0 equiv.) and compound 8 (758.10 mg, 2.459 mmol, 3.0 equiv.) in dichloromethane (4.1 mL) was added DIPEA (0.428 mL, 2.459 mmol, 3.0 equiv.). The resulting solution was stirred at RT until full conversion by LC-MS. The reaction mixture was diluted with water (10 mL). The product was extracted with dichloromethane (3×20 mL). The combined organic phase washed with brine (1×20 mL), dried over anhydrous Na₂SO₄, filtered, and concentrated under vacuum. The residue was purified by Combiflash® using silica gel as the stationary phase with a gradient of 0-40% methanol in dichloromethane, in which the product eluted at 35% B. The product was concentrated under vacuum to provide a white solid. LC-MS: calculated [M+H]+ 853.59, observed 853.62.

### Synthesis of Compound 9c

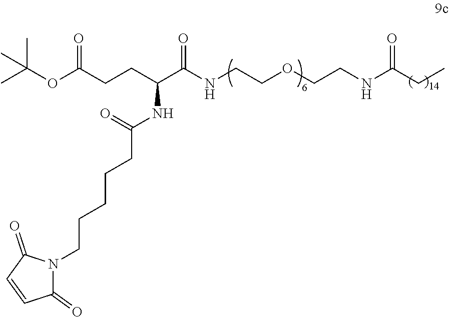

To a solution of compound 7c (511.00 mg, 0.683 mmol, 1.0 equiv.) and compound 8 (421.00 mg, 1.37 mmol, 2.0 equiv.) in dichloromethane (6.8 mL) was added DIPEA (0.595 mL, 3.416 mmol, 5.0 equiv.). The resulting solution was stirred at RT until full conversion by LC-MS. The reaction mixture was diluted with water (10 mL). The product was extracted with dichloromethane (3×20 mL). The combined organic phase washed with brine (1×20 mL), dried over anhydrous Na₂SO₄, filtered, and concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum to provide a colorless solid. LC-MS: calculated [M+H]+ 941.64, observed 941.66.

### Synthesis of Compound 9d

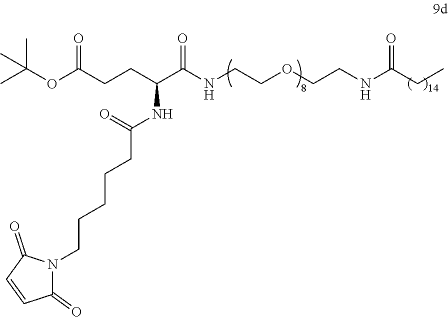

To a solution of compound 7d (573.00 mg, 0.685 mmol, 1.0 equiv.) and compound 8 (633.79 mg, 2.056 mmol, 3.0 equiv.) in dichloromethane (3.4 mL) was added DIPEA (0.596 mL, 3.426 mmol, 5.0 equiv.). The resulting solution was stirred at RT until full conversion by LC-MS. The reaction mixture was diluted with water (10 mL). The product was extracted with dichloromethane (3×20 mL). The combined organic phase washed with brine (1×20 mL), dried over anhydrous Na₂SO₄, filtered, and concentrated under vacuum. The residue was purified by Combiflash® using silica gel as the stationary phase with a gradient of 0-80%3:1 ethyl acetate:ethanol in heptane, in which the product eluted at 80% B. The product was concentrated under vacuum to provide a white foamy solid LC-MS: calculated [M+H]+ 1029.70, observed 1029.71.

### Synthesis of Compound 9e

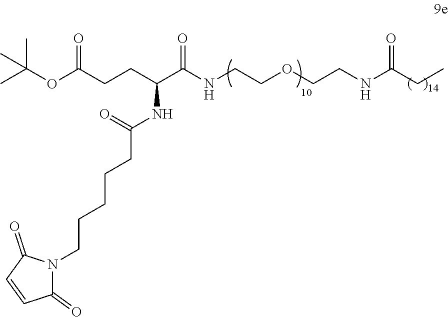

To a solution of compound 7e (179.00 mg, 0.194 mmol, 1.0 equiv.) and compound 8 (119.00 mg, 0.387 mmol, 2.0 equiv.) in dichloromethane (3.4 mL) was added DIPEA (0.169 mL, 0.968 mmol, 5.0 equiv.). The resulting solution was stirred at RT until full conversion by LC-MS. The reaction mixture was diluted with water (10 mL). The product was extracted with dichloromethane (3×20 mL). The combined organic phase washed with brine (1×20 mL), dried over anhydrous Na₂SO₄, filtered, and concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum to provide a white solid LC-MS: calculated [M+H]+ 1117.75, observed 1117.75.

### Synthesis of Compound 9f

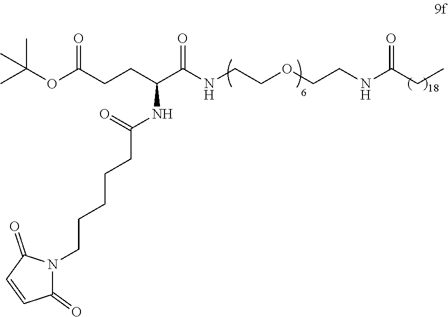

To a solution of compound 7f (390.84 mg, 0.486 mmol, 1.0 equiv.) and compound 8 (299.67 mg, 0.972 mmol, 2.0 equiv.) in dichloromethane (4.9 mL) was added DIPEA (0.423 mL, 2.430 mmol, 5.0 equiv.). The resulting solution was stirred at RT until full conversion by LC-MS. The reaction mixture was diluted with water (10 mL). The product was extracted with dichloromethane (3×20 mL). The combined organic phase washed with brine (1×20 mL), dried over anhydrous Na₂SO₄, filtered, and concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum to provide a white solid LC-MS: calculated [M+H]+ 997.71, observed 997.72.

### Synthesis of Compound 9g

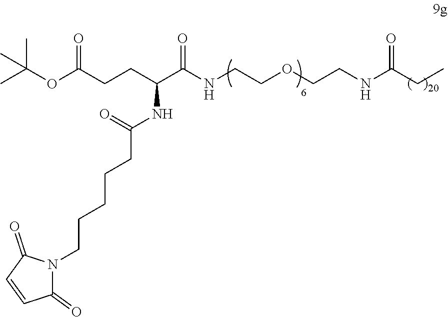

To a solution of compound 7g (427.75 mg, 0.514 mmol, 1.0 equiv.) and compound 8 (316.92 mg, 1.028 mmol, 2.0 equiv.) in dichloromethane (5.1 mL) was added DIPEA (0.447 mL, 2.570 mmol, 5.0 equiv.). The resulting solution was stirred at RT until full conversion by LC-MS. The reaction mixture was diluted with water (10 mL). The product was extracted with dichloromethane (3×20 mL). The combined organic phase washed with brine (1×20 mL), dried over anhydrous Na₂SO₄, filtered, and concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum to provide a white solid LC-MS: calculated [M+H]+ 1025.74, observed 1025.75.

### Synthesis of Compound 9h

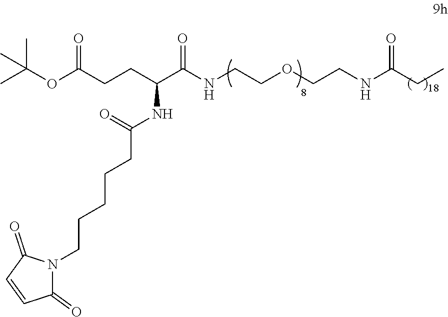

To a solution of compound 7h (140.00 mg, 0.157 mmol, 1.0 equiv.) and compound 8 (145.11 mg, 0.471 mmol, 3.0 equiv.) in dichloromethane (1.6 mL) was added DIPEA (0.136 mL, 0.785 mmol, 5.0 equiv.). The resulting solution was stirred at RT until full conversion by LC-MS. The reaction mixture was diluted with water (10 mL). The product was extracted with dichloromethane (3×20 mL). The combined organic phase washed with brine (1×20 mL), dried over anhydrous Na₂SO₄, filtered, and concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum to provide a white solid LC-MS: calculated [M+H]+ 1085.76, observed 1085.75.

### Synthesis of Compound 9i

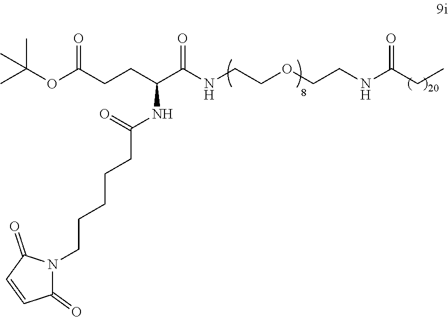

To a solution of compound 7i (245.00 mg, 0.266 mmol, 1.0 equiv.) and compound 8 (246.22 mg, 0.799 mmol, 3.0 equiv.) in dichloromethane (2.7 mL) was added DIPEA (0.231 mL, 1.331 mmol, 5.0 equiv.). The resulting solution was stirred at RT until full conversion by LC-MS. The reaction mixture was diluted with water (10 mL). The product was extracted with dichloromethane (3×20 mL). The combined organic phase washed with brine (1×20 mL), dried over anhydrous Na₂SO₄, filtered, and concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum to provide a white solid LC-MS: calculated [M+H]+ 1113.79, observed 1113.77.

### Synthesis of Compound 9j

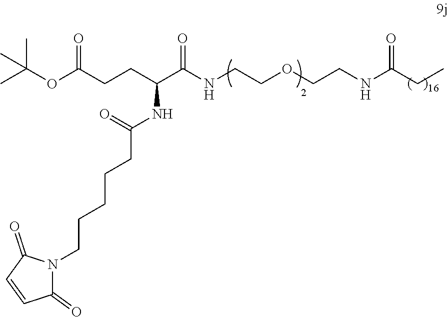

To a solution of compound 7j (50.00 mg, 0.084 mmol, 1.0 equiv.) and compound 8 (77.00 mg, 0.251 mmol, 3.0 equiv.) in dichloromethane (0.8 mL) was added DIPEA (0.044 mL, 0.251 mmol, 3.0 equiv.). The resulting solution was stirred at RT until full conversion by LC-MS. The reaction mixture was diluted with water (10 mL). The product was extracted with dichloromethane (3×20 mL). The combined organic phase washed with brine (1×20 mL), dried over anhydrous Na₂SO₄, filtered, and concentrated under vacuum. The residue was purified by Combiflash® using silica gel as the stationary phase with a gradient of 0-80% 3:1 ethyl acetate:ethanol in heptane, in which the product eluted at 80% B. The product was concentrated under vacuum to provide a colorless solid LC-MS: calculated [M+H]+ 793.57, observed 793.61.

### Synthesis of Compound 10a

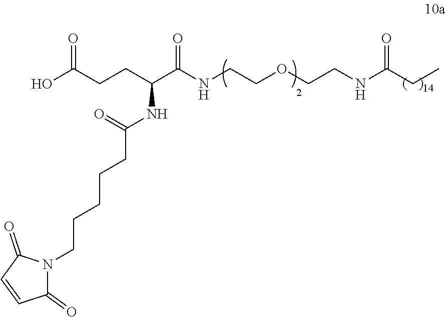

To compound 9a (570.00 mg, 0.745 mmol) was added 1:1 TFA:dichloromethane (3.0 mL). The reaction mixture was stirred at RT until full conversion by LC-MS. The product was concentrated under vacuum overnight. No isolation was necessary. Concentration provided a yellow solid. LC-MS: calculated [M+H]+ 709.47, observed 709.59.

### Synthesis of Compound 10b

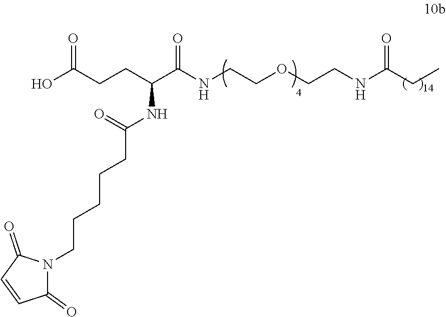

To compound 9b (505.00 mg, 0.592 mmol) was added 1:1 TFA:dichloromethane(1.8 mL). The reaction mixture was stirred at RT until full conversion by LC-MS. The product was concentrated under vacuum overnight. No isolation was necessary. Concentration provided an off white solid. LC-MS: calculated [M+Na]+819.51, observed 819.55.

### Synthesis of Compound 10c

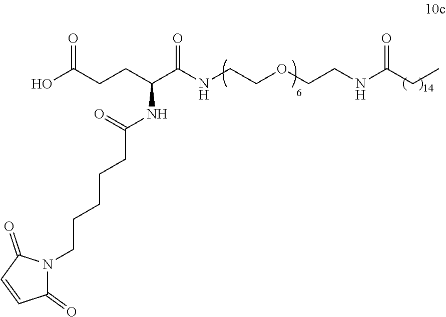

To compound 9c (418.00 mg, 0.444 mmol) was added 1:1 TFA:dichloromethane(2.0 mL). The reaction mixture was stirred at RT until full conversion by LC-MS. The product was concentrated under vacuum overnight. No isolation was necessary. Concentration provided an off white solid. LC-MS: calculated [M+Na]+907.56, observed 907.58.

### Synthesis of Compound 10d

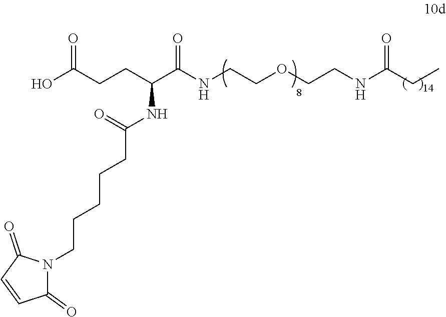

To compound 9d (653.70 mg, 0.635 mmol) was added 1:1 TFA:dichloromethane(2.0 mL). The reaction mixture was stirred at RT until full conversion by LC-MS. The product was concentrated under vacuum overnight. No isolation was necessary. Concentration provided a colorless solid LC-MS: calculated [M+H]+ 973.63, observed 973.64.

### Synthesis of Compound 10e

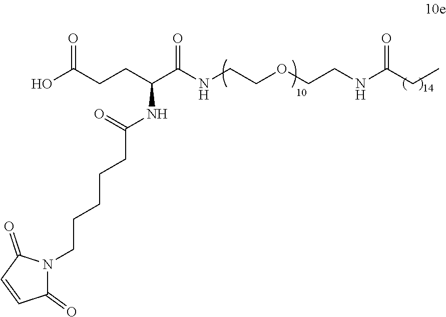

To compound 9e (138.00 mg, 0.123 mmol) was added 1:1 TFA:dichloromethane(0.5 mL). The reaction mixture was stirred at RT until full conversion by LC-MS. The product was concentrated under vacuum overnight. No isolation was necessary. Concentration provided a colorless solid. LC-MS: calculated [M+Na]+1083.67, observed 1083.67.

### Synthesis of Compound 10f

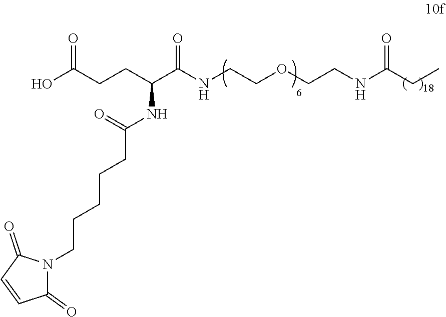

To compound 9f (109.80 mg, 0.110 mmol) was added 1:1 TFA:dichloromethane(1.1 mL). The reaction mixture was stirred at RT until full conversion by LC-MS. The product was concentrated under vacuum overnight. No isolation was necessary. Concentration provided a colorless solid. LC-MS: calculated [M+H]+ 941.64, observed 941.65.

### Synthesis of Compound 10g

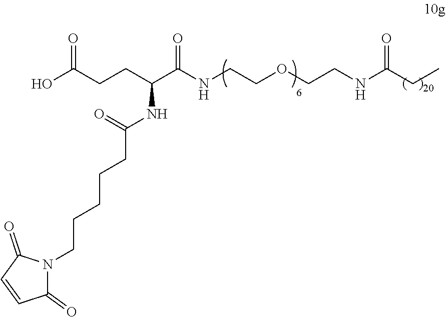

To compound 9g (258.70 mg, 0.252 mmol) was added 1:1 TFA:dichloromethane(2.5 mL). The reaction mixture was stirred at RT until full conversion by LC-MS. The product was concentrated under vacuum overnight. No isolation was necessary. Concentration provided a colorless solid. LC-MS: calculated [M+Na]+991.66, observed 991.65.

### Synthesis of Compound 10h

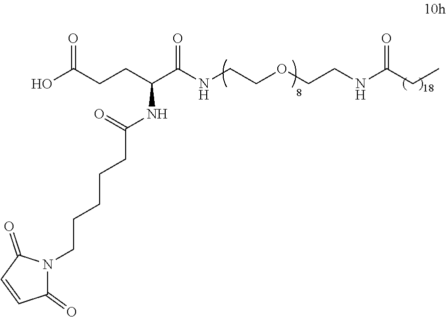

To compound 9h (109.80 mg, 0.101 mmol) was added 1:1 TFA:dichloromethane (1.0 mL). The reaction mixture was stirred at RT until full conversion by LC-MS. The product was concentrated under vacuum overnight. No isolation was necessary. Concentration provided an off white solid. LC-MS: calculated [M+Na]+1051.68, observed 1051.66.

### Synthesis of Compound 10i

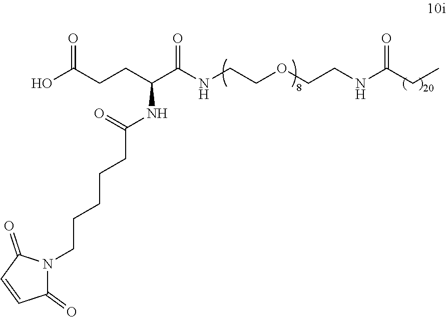

To compound 9i (275.00 mg, 0.247 mmol) was added 1:1 TFA:dichloromethane(2.5 mL). The reaction mixture was stirred at RT until full conversion by LC-MS. The product was concentrated under vacuum overnight. No isolation was necessary. Concentration provided a colorless solid. LC-MS: calculated [M+Na]+1079.71, observed 1079.69.

### Synthesis of Compound 10j

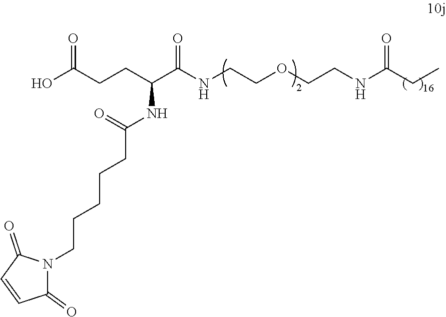

To compound 9j (43.00 mg, 0.054 mmol) was added 1:1 TFA:dichloromethane(0.2 mL). The reaction mixture was stirred at RT until full conversion by LC-MS. The product was concentrated under vacuum overnight. No isolation was necessary. Concentration provided a yellow solid. LC-MS: calculated [M+Na]+759.49, observed 759.52.

### Synthesis of Compound 12a

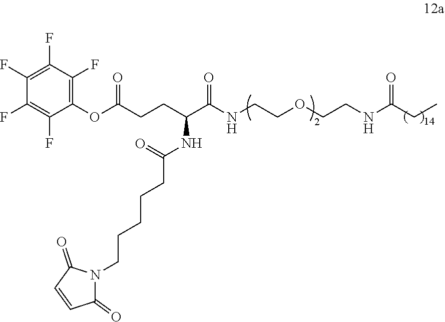

To a solution of compound 10a (500.00 mg, 0.705 mmol, 1.0 equiv.) and compound 11 (389.44 mg, 2.116 mmol, 3.0 equiv.) in dichloromethane (7 mL) was added DIC (0.328 mL, 2.116 mmol, 3.0 equiv.). The resulting solution was stirred at RT until full conversion by LC-MS. The resulting solution was concentrated under vacuum. The residue was purified by Combiflash® using silica gel as the stationary phase with a gradient of 0-80% 3:1 Ethyl acetate:ethanol in heptane, in which the product eluted at 80% B. The product was concentrated under vacuum and provided an off white solid. LC-MS: calculated [M+H]+ 875.46, observed 875.43.

### Synthesis of Compound 12b

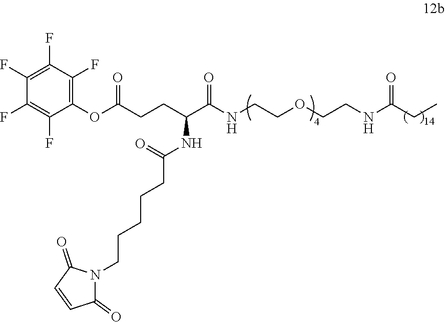

To a solution of compound 10b (100.00 mg, 0.126 mmol, 1.0 equiv.) and compound 11 (69.28 mg, 0.376 mmol, 3.0 equiv.) in dichloromethane (1.2 mL) was added DIC (0.058 mL, 0.376 mmol, 3.0 equiv.). The resulting solution was stirred at RT until full conversion by LC-MS. The resulting solution was concentrated under vacuum. The residue was purified by Combiflash® using silica gel as the stationary phase with a gradient of 0-80%3:1 Ethyl acetate ethanol in heptane, in which the product eluted at 80% B. The product was concentrated under vacuum and provided an off white solid. LC-MS: calculated [M+Na]+985.49, observed 985.51.

### Synthesis of Compound 12c

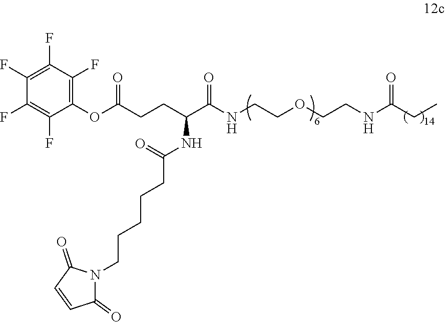

To a solution of compound 10c 100.00 mg, 0.113 mmol, 1.0 equiv.) and compound 11 (42.00 mg, 0.226 mmol, 2.0 equiv.) in dichloromethane (1.2 mL) was added DIC (0.035 mL, 0.226 mmol, 2.0 equiv.). The resulting solution was stirred at RT until full conversion by LC-MS. The resulting solution was concentrated under vacuum. The residue was purified by Combiflash® using silica gel as the stationary phase with a gradient of 0-80% 3:1 Ethyl acetate ethanol in heptane, in which the product eluted at 80% B. The product was concentrated under vacuum and provided an off white solid. LC-MS: calculated [M+Na]+1073.55, observed 1073.56.

### Synthesis of Compound 12d

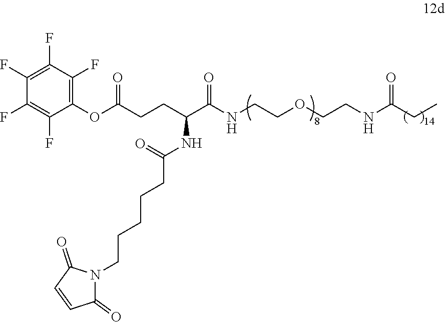

To a solution of compound 10d (100.00 mg, 0.103 mmol, 1.0 equiv.) and compound 11 (37.82 mg, 0.206 mmol, 2.0 equiv.) in dichloromethane (1.0 mL) was added DIC (0.032 mL, 0.206 mmol, 2.0 equiv.). The resulting solution was stirred at RT until full conversion by LC-MS. The resulting solution was concentrated under vacuum. The residue was purified by Combiflash® using silica gel as the stationary phase with a gradient of 0-80% 3:1 Ethyl acetate ethanol in heptane, in which the product eluted at 80% B. The product was concentrated under vacuum and provided an off white solid. LC-MS: calculated [M+H]+ 1139.62, observed 1139.61.

### Synthesis of Compound 12e

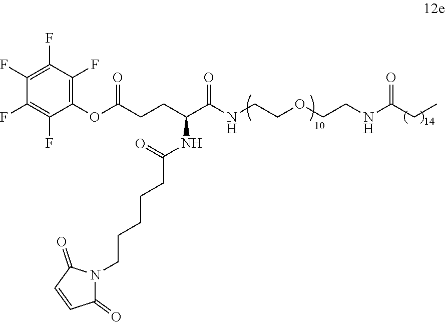

To a solution of compound 10e (50.00 mg, 0.047 mmol, 1.0 equiv.) and compound 11 (13.00 mg, 0.071 mmol, 1.5 equiv.) in dichloromethane (0.5 mL) was added DIC (0.011 mL, 0.071 mmol, 1.5 equiv.). The resulting solution was stirred at RT until full conversion by LC-MS. The resulting solution was concentrated under vacuum. The residue was purified by Combiflash® using silica gel as the stationary phase with a gradient of 0-80% 3:1 Ethyl acetate ethanol in heptane, in which the product eluted at 80% B. The product was concentrated under vacuum and provided an off white solid. LC-MS: calculated [M+H]+ 1227.67, observed 1227.66.

### Synthesis of Compound 12f

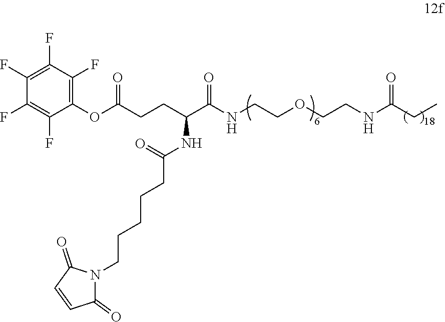

To a solution of compound 10f (100.00 mg, 0.106 mmol, 1.0 equiv.) and compound 11 (58.66 mg, 0.319 mmol, 3.0 equiv.) in dichloromethane (1.1 mL) was added DIC (0.049 mL, 0.319 mmol, 3.0 equiv.). The resulting solution was stirred at RT until full conversion by LC-MS. The resulting solution was concentrated under vacuum. The residue was purified by Combiflash® using silica gel as the stationary phase with a gradient of 0-80% 3:1 Ethyl acetate ethanol in heptane, in which the product eluted at 80% B. The product was concentrated under vacuum and provided an off white solid. LC-MS: calculated [M+H]+ 1107.63, observed 1107.64.

### Synthesis of Compound 12g

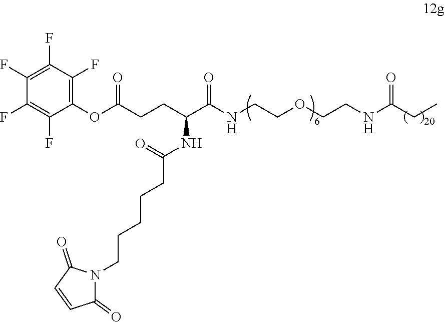

To a solution of compound 10g (100.00 mg, 0.103 mmol, 1.0 equiv.) and compound 11 (56.97 mg, 0.310 mmol, 2.0 equiv.) in dichloromethane (0.5 mL) was added DIC (0.048 mL, 0.310 mmol, 2.0 equiv.). The resulting solution was stirred at RT until full conversion by LC-MS. The resulting solution was concentrated under vacuum. The residue was purified by Combiflash® using silica gel as the stationary phase with a gradient of 0-80% 3:1 Ethyl acetate ethanol in heptane, in which the product eluted at 80% B. The product was concentrated under vacuum and provided an off white solid. LC-MS: calculated [M+H]+ 1135.66, observed 1135.67.

### Synthesis of Compound 12h

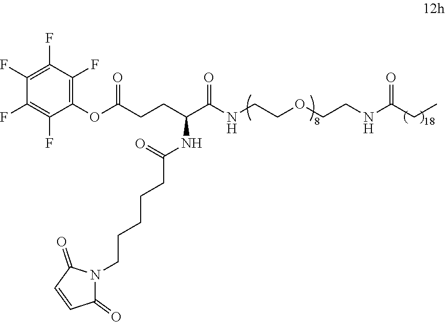

To a solution of compound 10h (100.00 mg, 0.097 mmol, 1.0 equiv.) and compound 11 (53.64 mg, 0291 mmol, 3.0 equiv.) in dichloromethane (1.0 mL) was added DIC (0.045 mL, 0.291 mmol, 3.0 equiv.). The resulting solution was stirred at RT until full conversion by LC-MS. The resulting solution was concentrated under vacuum. The residue was purified by Combiflash® using silica gel as the stationary phase with a gradient of 0-80% 3:1 Ethyl acetate ethanol in heptane, in which the product eluted at 80% B. The product was concentrated under vacuum and provided an off white solid. LC-MS: calculated [M+H]+ 1195.68, observed 1195.65.

### Synthesis of Compound 12i

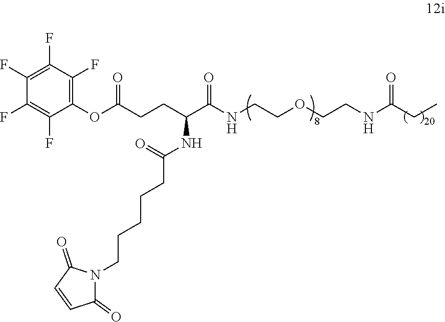

To a solution of compound 10i (100.00 mg, 0.95 mmol, 1.0 equiv.) and compound 11 (52.22 mg, 0.284 mmol, 3.0 equiv.) in dichloromethane (1.0 mL) was added DIC (0.044 mL, 0.284 mmol, 3.0 equiv.). The resulting solution was stirred at RT until full conversion by LC-MS. The resulting solution was concentrated under vacuum. The residue was purified by Combiflash® using silica gel as the stationary phase with a gradient of 0-80% 3:1 Ethyl acetate ethanol in heptane, in which the product eluted at 80% B. The product was concentrated under vacuum and provided an off white solid. LC-MS: calculated [M+H]+ 1223.71, observed 1223.68.

### Synthesis of Compound 12j

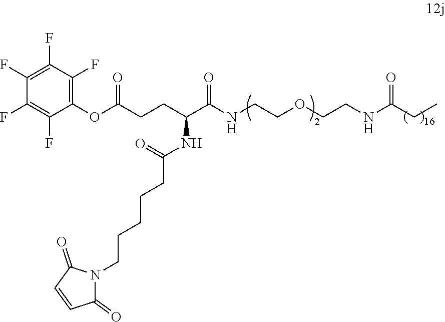

To a solution of compound 10j (20.00 mg, 0.027 mmol, 1.0 equiv.) and compound 11 (5.00 mg, 0.030 mmol, 1.1 equiv.) in dichloromethane (0.27 mL) was added DIC (0.005 mL, 0.30 mmol, 1.1 equiv.). The resulting solution was stirred at RT until full conversion by LC-MS. The resulting solution was concentrated under vacuum. The residue was purified by Combiflash® using silica gel as the stationary phase with a gradient of 0-80% 3:1 Ethyl acetate:ethanol in heptane, in which the product eluted at 80% B. The product was concentrated under vacuum and provided an off white solid. LC-MS: calculated [M+H]+ 903.49, observed 903.51.

### Conjugation of Lipid Linkers 12a-j

Either prior to or after annealing, the 5′- or 3′-amine functionalized sense strand is conjugated to a lipid linker, 12a-j, directly. The following generally describes the conjugation of activated ester functionalized linkers 12a-j. Amine-functionalized duplex was dissolved in 70% DMSO/30%100 mM PB in pH 8.0 at ~10 mg/mL. 10 equivalents of linker were added. The reaction was monitored by LC-MS. Once complete, the reaction mixture was diluted with water and filtered to remove excess linker. The conjugate was buffer exchanged into 10 mM NaOAc pH 6.0 via dialysis.

### Synthesis of Linkers 15a-g

Linkers 15a-g were prepared by modification of compounds 13a-g which were obtained using general Fmoc peptide chemistry utilizing Fmoc-Lys(TFA)-CO₂H preloaded 2-Cl-Trt resin (0.41 mmol/g) at 0.3 mmol scale. Following cleavage from resin, compounds 14a-g were purified via flash column chromatography with a gradient of 0-10% methanol in 99:1 dichloromethane:acetic acid.

Compounds 14a-h were then converted into pentafluorophenyl esters 15a-h. The crude products were purified via flash column chromatography with a gradient of 0-80% 3:1 ethyl acetate:ethanol in heptane.

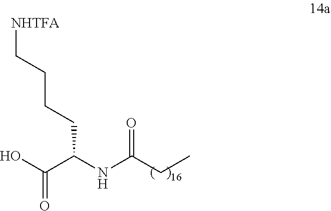

Compound 14a was isolated as a white solid. LC-MS: calculated [M+Na]+531.34, observed 531.34.

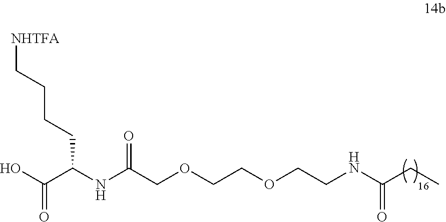

Compound 14b was isolated as a colorless solid. LC-MS: calculated [M+H]+ 654.43, observed 654.44.

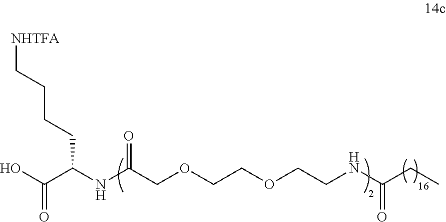

Compound 14c was isolated as a colorless solid. LC-MS: calculated [M+H]+ 799.50, observed 799.50.

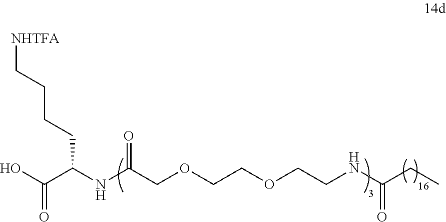

Compound 14d was isolated as a colorless oil. LC-MS: calculated [M+H]+ 944.58, observed 944.60.

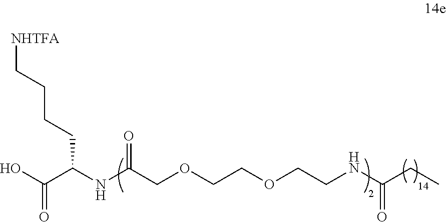

Compound 14e was isolated as a colorless oil. LC-MS: calculated [M+H]+ 771.47, observed 771.48.

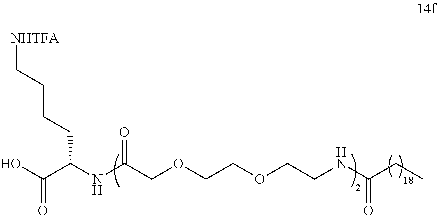

Compound 14f was isolated as a colorless solid. LC-MS: calculated [M+H]+ 827.54, observed 827.55.

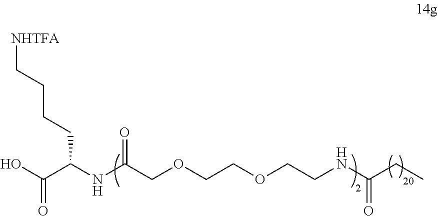

Compound 14g was isolated as a white solid. LC-MS: calculated [M+H]+ 855.57, observed 855.59.

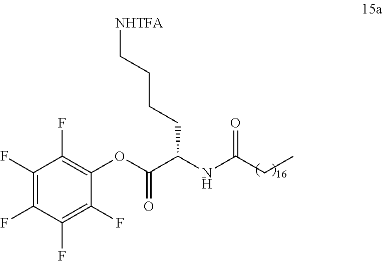

Compound 15a was isolated as a white solid. LC-MS: calculated [M+H]+ 675.34, observed 675.36.

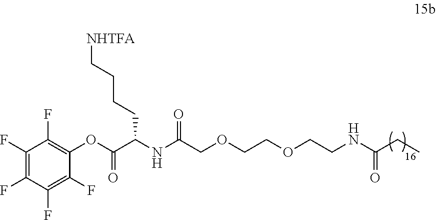

Compound 15b was isolated as a white solid. LC-MS: calculated [M+H]+ 820.41, observed 820.43.

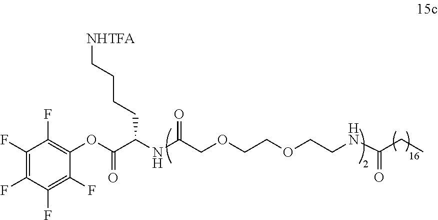

Compound 15c was isolated as an off white solid. LC-MS: calculated [M+H]+ 965.49, observed 965.50.

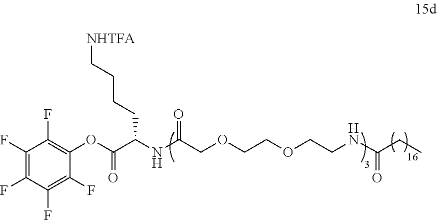

Compound 15d was isolated as an off white solid. LC-MS: calculated [M+H]+ 1110.56, observed 1110.57.

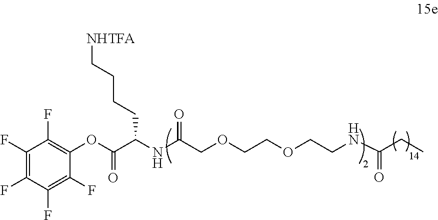

Compound 15e was isolated as an off white solid. LC-MS: calculated [M+H]+ 937.46, observed 937.47.

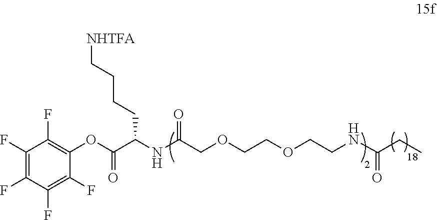

Compound 15f was isolated as a white solid. LC-MS: calculated [M+H]+ 993.52, observed 993.53.

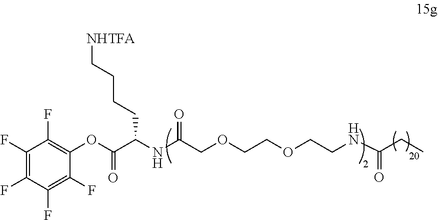

Compound 15g was isolated as a white solid. LC-MS: calculated [M+H]+ 1021.55, observed 1021.57.

### Conjugation of Lipid Linkers 15a-g

Either prior to or after annealing, the 5′- or 3′-amine functionalized sense strand is conjugated to a lipid linker, 15a-g, directly. The following generally describes the conjugation of activated ester functionalized linkers 12a-j. Amine-functionalized duplex was dissolved in 9:1:0.5 DMSO:100 mM Borate Buffer pH 8.5:triethylamine at ~50 mg/mL. 10 equivalents of linker were added. The reaction was monitored by LC-MS. Once complete, the reaction mixture was diluted with water and filtered. To the filtrate was added AMA solution (10% by volume) and the resulting solution was incubated at RT for 2 hours. The reaction mixture was concentrated under a stream of air. Conjugates were purified by non-denaturing anion exchange chromatography by method 2 and lyophilized.

Conjugation of Compound 15a-g Modified Duplexes with SMCC

The following generally describes the conjugation of NHS-SMCC with duplexes modified with compound 15a-g. Modified duplexes were dissolved in 70% DMSO/30%50 mM PB pH 7.6.8 equivalents of SMCC-NHS was added. The reactions were monitored via LC-MS. Upon completion, the reaction mixture was diluted with water and filtered. The conjugates were buffer exchanged into 10 mM NaOAc pH 6.0 via dialysis.

### Conjugate Synthesis and Characterization

The CD71 Fab-siRNA conjugates were made, purified and characterized as described in Example 3. All conjugates were made through cysteine conjugation with a SMCC linker or with lipid-linker as described. Conjugates were characterized chromatographically as described in Table 47.

TABLE 47
HPLC Retention Time (RT) in Minutes

| Group(s) | Conjugate | RT, SAX Method-5 | RT, SEC Method-2 |
|---|---|---|---|
| 1 | mTfR1-Fab-siDMPK DAR1 | 3.449 | 14.983 |
| 2 | mTfR1-Fab- | 3.602 | 15.481 |
| | pos3.C22.siDMPK DAR1 | | |
| 3 | mTfR1-Fab-PEG2 C16 linker- | 3.545 | 14.930 |
| | siDMPK DAR1 | | |
| 4 | mTfR1-Fab-PEG6 C16 linker- | 3.497 | 14.978 |
| | siDMPK DAR1 | | |
| 5 | mTfR1-Fab-PEG10 C16 | — | — |
| | linker-siDMPK DAR1 | | |
| 6 | mTfR1-Fab-PEG6 C20 linker- | 3.514 | 15.045 |
| | siDMPK DAR1 | | |
| 7 | mTfR1-Fab-PEG6 C22 linker- | 3.545 | 15.062 |
| | siDMPK DAR1 | | |

### In Vivo Study Design

The conjugates were assessed for their ability to mediate mRNA downregulation of myotonic dystrophy protein kinase (DM PK) in skeletal muscle in vivo in humanized TfR1 mice. Mice were dosed via intravenous (IV) injection or subcutaneous (SC) with PBS vehicle control or the indicated AOCs at 0.3 mg/kg siRNA dose as shown in Table 48. After 28 days, gastrocnemius (gastroc) and heart muscle tissues were harvested and snap-frozen in liquid nitrogen. mRNA knockdown in target tissue was determined using a comparative PCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 48
In vivo study design testing duration of action
of DMPK siRNA Fab AOCs in wild type mice

| | | | | siRNA Dose | Harvest |
|---|---|---|---|---|---|
| Group | Test Article | N | DAR | (mg/kg) | (day) |
| 1 | mTfR1-Fab-siDMPK DAR1 | 4 | 1 | 0.3 | 1 |
| 2 | mTfR1-Fab-pos3.C22.siDMPK DAR1 | 4 | 1 | 1 | 2 |
| 3 | mTfR1-Fab-PEG2 C16 linker-siDMPK DAR1 | 4 | 1 | 1 | 3 |
| 4 | mTfR1-Fab-PEG6 C16 linker-siDMPK DAR1 | 4 | 1 | 1 | 4 |
| 5 | mTfR1-Fab-PEG10 C16 linker-siDMPK DAR1 | 4 | 1 | 1 | 5 |
| 6 | mTfR1-Fab-PEG6 C20 linker-siDMPK DAR1 | 4 | 1 | 1 | 6 |
| 7 | mTfR1-Fab-PEG6 C22 linker-siDMPK DAR1 | 4 | 1 | 1 | 7 |
| 8 | PBS | 4 | N/A | N/A | 8 |

* All animals used in experiments shown in Table 48 were injected at a single dose volume of 28 ml/kg by IV administration.

TABLE 49
siRNA Sequences used in Example 16

| | | | SEQ | | SEQ |
|---|---|---|---|---|---|
| Compound | | | ID | | ID |
| No. | siRNA Ref. | siRNA Guide Sequence | NO: | SiRNA Passenger Sequence | NO: |
| 1 | (vp)siDMPK | [5vp_una2moe](U)[ps].[fl2r](A) | 12 | [5am6_p_m](C)[ps].m(C)[ps].m | 48 |
| | | [ps].m(A)p.m(U)p.m(A)p.[f12r] | | (G)p.m(A)p.m(C)p.m(A)p.[fl2r] | |
| | | (C)p.m(C)p.m(G)p.m(A)p.m(G) | | (U)p.[fl2r](U)p.[fl2r](C)p.m(C)p. | |
| | | p.m(G)p.m(A)p.m(A)p.[fl2r](U) | | m(U)p.m(C)p.m(G)p.m(G)p.m | |
| | | p.m(G)p.[fl2r](U)p.m(C)p.m(G) | | (U)p.m(A)p.m(U)[ps].m(U)[ps]. | |
| | | p.m(G)[ps].m(U)[ps].m(U) | | m(A) | |
| 2 | Pos3.C22.siD | [5vp_una2moe](U)[ps].[fl2r](A) | 12 | [5am6_p_m](C)[ps].m(C)[ps].[r | 81 |
| | MPK | [ps].m(A)p.m(U)p.m(A)p.[fl2r] | | c22](G)p.m(A)p.m(C)p.m(A)p.[f | |
| | | (C)p.m(C)p.m(G)p.m(A)p.m(G)p.m | | 12r](U)p.[f12r](U)p.[f12r](C)p.m | |
| | | (G)p.m(A)p.m(A)p.[fl2r](U)p.m | | (C)p.m(U)p.m(C)p.m(G)p.m(G) | |
| | | (G)p.[fl2r](U)p.m(C)p.m(G)p.m | | p.m(U)p.m(A)p.m(U)[ps].m(U)[p | |
| | | (G)[ps].m(U)[ps].m(U) | | s].m(A) | |

5vp_una2moe 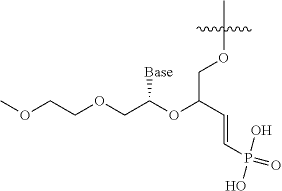
5am6_p_m 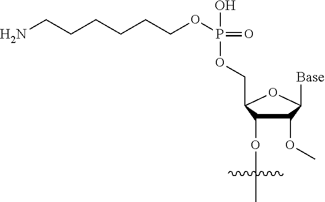
Rc22

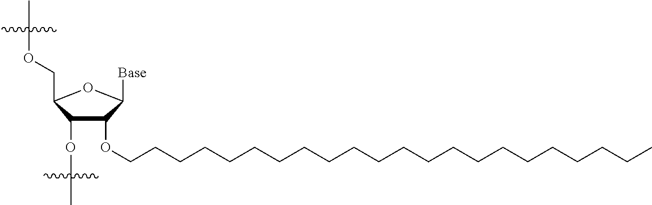

In Table 49, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

### Results

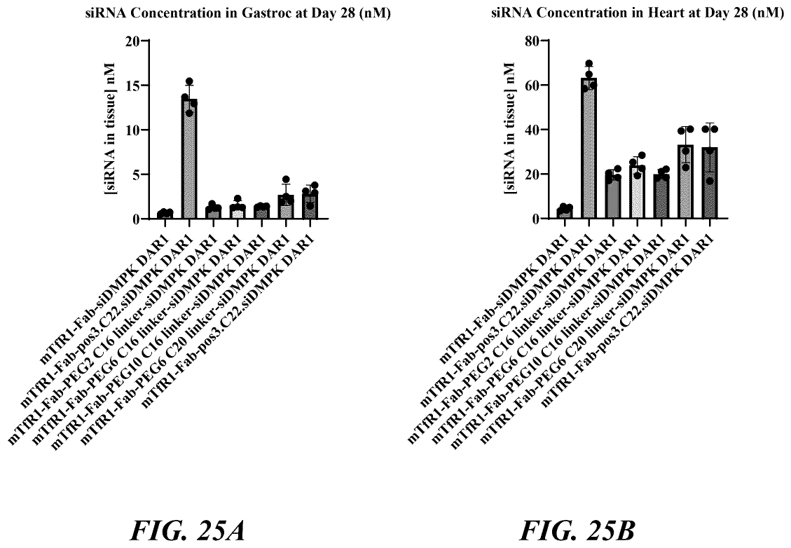

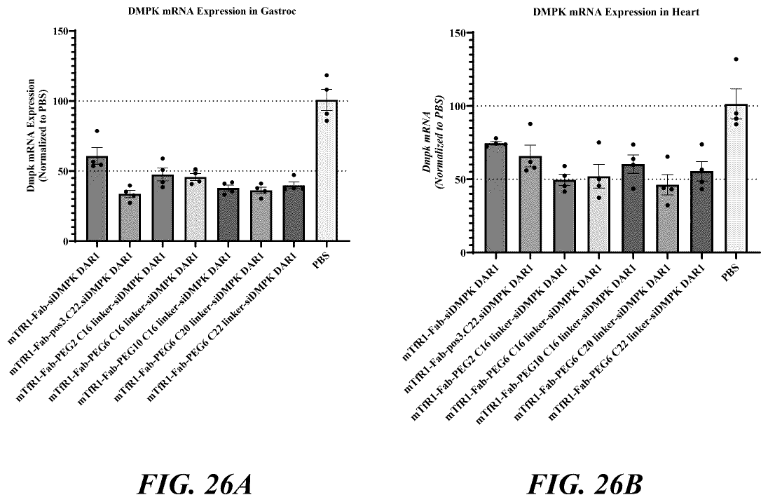

As shown in FIGS. 25A-25B and 26A-26B, DMPK siRNA tissue concentrations and DMPK mRNA expression levels were assessed with DAR1 lipid-FabOC with the C22 hydrocarbon chain conjugated at position 3 passenger strand or with the lipid conjugated to the linker of the FabOC at day 28 in the gastroc (gastroc), and heart obtained from mice following a single intravenous (IV) injection of the DAR1 FabOCs at Day 0.

At day 28 DMPK siRNA tissue concentrations were quantified in tissue obtained from wild type mice that were administered a single intravenous injection of DAR1 lipid FabOCs with C22 hydrocarbon chain conjugated at position 3 nucleotide of the passenger strand or DAR1 lipid FabOCs with C16, C20, or C22 hydrocarbon chain conjugated to the linker linking the Fab to the siRNA with a polyethylene glycol (PEG) spacer as specified.

As shown in FIGS. 25A-25B, all lipid FabOCs were able to deliver higher amount of siRNA to all muscle tissues than the non-lipid FabOC. The lipid FabOCs with the lipid conjugated to the linkers delivered similar amounts of siRNA in the heart and gastroc muscle tissues. However, the lipid FabOCs with the lipid conjugated to the linkers were delivered less than the DAR1 lipid FabOCs with C22 hydrocarbon chain conjugated at position 3 nucleotide of the passenger strand (mTfR1-Fab-(vp)pos6.C22.siDMPK DAR1).

As shown in FIG. 26A-26B, all lipid FabOCs were able to reduce mRNA expression levels more than non-lipid FabOC in all muscle tissues.

Overall, the lipid FabOCs with C16, 20, or 22 hydrocarbon chains conjugated to the linker have similar biological activities as the lipid FabOCs with C22 hydrocarbon chain conjugated to the passenger strand of the siRNA.

### Example 17: Duration of Action of TfR1-Targeted DAR1, DAR2, and DAR4 siRNA AOCs in Wild Type Mice

DMPK: A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker.

### Conjugate Synthesis and Characterization

The CD71 mAb-siRNA conjugates were made, purified and characterized as described in Example 3. All conjugates were made through cysteine conjugation with a SMCC linker. Conjugates were characterized chromatographically as described in Table 50.

TABLE 50
HPLC Retention Time (RT) in Minutes

| | | RT, SAX | RT, SEC |
|---|---|---|---|
| Group(s) | Conjugate | Method-5 | Method-2 |
| 1 | mTfR1-mAb-siDMPK DAR1 | 3.13 | 2.89 |
| 2 | mTfR1-mAb-pos3.C22.siDMPK DAR1 | 3.169 | 2.942 |
| 3 | mTfR1-mAb-pos6.C16.siDMPK DAR1 | 3.136 | 2.933 |
| 4 | mTfR1-mAb-pos6.C16.siDMPK DAR2 | 3.601 | 2.846 |
| 5 | mTfR1-mAb-pos6.C16.siDMPK DAR4 | 4.105 | 2.669 |

### In Vivo Study Design

The conjugates were assessed for their ability to mediate mRNA downregulation of myotonic dystrophy protein kinase (DMPK) in skeletal muscle in vivo in humanized TfR1 mice. Mice were dosed via intravenous (IV) injection with PBS vehicle control or the indicated AOCs at 10 mg/kg mAb protein dose, or 1 mg/kg for the DAR1 conjugates and 2 mg/kg for the DAR2 and 4 mg/kg for the DAR4 conjugates as shown in Table 51. After 1, 3, and 5 months, gastrocnemius (gastroc) and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 51
In vivo study design testing duration of action of DAR1, DAR2, and DAR4
DMPK siRNA AOCs in wild type mice

| | | | | | siRNA | | |
|---|---|---|---|---|---|---|---|
| | | siRNA | | | Dose | Doses | Harvest |
| Group(s) | Test Article | Target | N | DAR | (mg/kg) | (#) | (month) |
| 1 | mTfR1-mAb-siDMPK DAR1 | DMPK | 4 | 1 | 1 | 1 | 1, 3, 5 |
| 2 | mTfR1-mAb- | DMPK | 4 | 1 | 1 | 1 | 1, 3, 5 |
| | pos3.C22.siDMPK DAR1 | | | | | | |
| 3 | mTfR1-mAb- | DMPK | 4 | 1 | 1 | 1 | 1, 3, 5 |
| | pos6.C16.siDMPK DAR1 | | | | | | |
| 4 | mTfR1-mAb- | DMPK | 4 | 2 | 2 | 1 | 1, 3, 5 |
| | pos6.C16.siDMPK DAR2 | | | | | | |
| 5 | mTfR1-mAb- | DMPK | 4 | 4.07 | 4.07 | 1 | 1, 3, 5 |
| | pos6.C16.siDMPK DAR4 | | | | | | |
| 6 | PBS | DMPK | 4 | N/A | N/A | 1 | 1, 3, 5 |

* All animals used in experiments shown in Table 51 have body weight of 0.035 kg, and the injection was made at a single dose volume of 5 ml/kg by IV administration.

TABLE 52
siRNA Sequences used in Example 17

| | | | SEQ | | SEQ |
|---|---|---|---|---|---|
| Compound | | | ID | | ID |
| No. | siRNA Ref. | siRNA Guide Sequence | NO: | siRNA Passenger Sequence | NO: |
| 1 | (vp)siDMPK | [5vp_una2moe](U)[ps].[fl2r](A) | 12 | [5am6_p_m](C)[ps].m(C)[ps].m | 48 |
| | | [ps].m(A)p.m(U)p.m(A)p.[fl2r] | | (G)p.m(A)p.m(C)p.m(A)p.[f12r] | |
| | | (C)p.m(C)p.m(G)p.m(A)p.m(G) | | (U)p.[fl2r](U)p.[fl2r](C)p.m(C)p. | |
| | | p.m(G)p.m(A)p.m(A)p.[fl2r](U) | | m(U)p.m(C)p.m(G)p.m(G)p.m | |
| | | p.m(G)p.[fl2r](U)p.m(C)p.m(G) | | (U)p.m(A)p.m(U)[ps].m(U)[ps]. | |
| | | p.m(G)[ps].m(U)[ps].m(U) | | m(A) | |
| 2 | Pos3.C22.siD | [5vp_una2moe](U)[ps].[fl2r](A) | 12 | [5am6_p_m](C)[ps].m(C)[ps].[r | 81 |
| | MPK | [ps].m(A)p.m(U)p.m(A)p.[fl2r] | | c22](G)p.m(A)p.m(C)p.m(A)p.[f | |
| | | (C)p.m(C)p.m(G)p.m(A)p.m(G) | | 12r](U)p.[f12r](U)p.[fl2r](C)p.m | |
| | | p.m(G)p.m(A)p.m(A)p.[fl2r](U) | | (C)p.m(U)p.m(C)p.m(G)p.m(G) | |
| | | p.m(G)p.[fl2r](U)p.m(C)p.m(G) | | p.m(U)p.m(A)p.m(U)[ps].m(U)[p | |
| | | p.m(G)[ps].m(U)[ps].m(U) | | s].m(A) | |
| 3 | (vp)pos6.C16.s | [5vp_una2moe](U)[ps].[fl2r](A) | 12 | [5am6_p_m](C)[ps].m(C)[ps].m | 49 |
| | IDMPK | [ps].m(A)p.m(U)p.m(A)p.[f12r] | | (G)p.m(A)p.m(C)p.[rc16](A)p.[f | |
| | | (C)p.m(C)p.m(G)p.m(A)p.m(G) | | 12r](U)p.[f12r](U)p.[fl2r](C)p.m | |
| | | p.m(G)p.m(A)p.m(A)p.[fl2r](U) | | (C)p.m(U)p.m(C)p.m(G)p.m(G) | |
| | | p.m(G)p.[fl2r](U)p.m(C)p.m(G) | | p.m(U)p.m(A)p.m(U)[ps].m(U)[p | |
| | | p.m(G)[ps].m(U)[ps].m(U) | | s].m(A) | |

5vp_una2moe 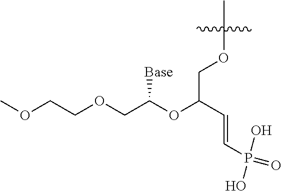
5am6_p_m 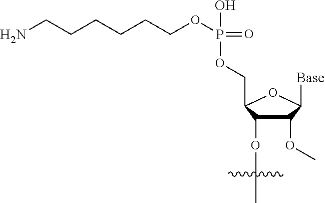
Rc22 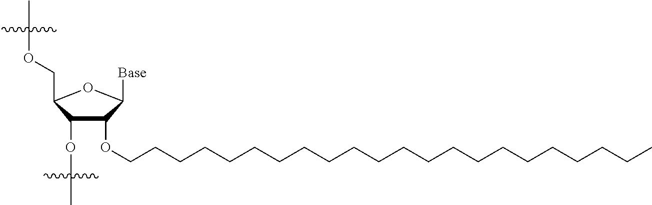
Rc16

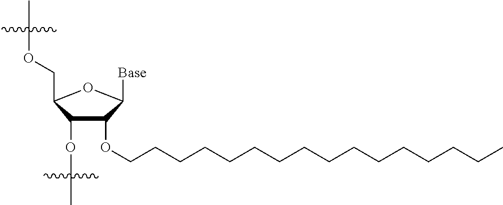

In Table 52, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage.

### Results

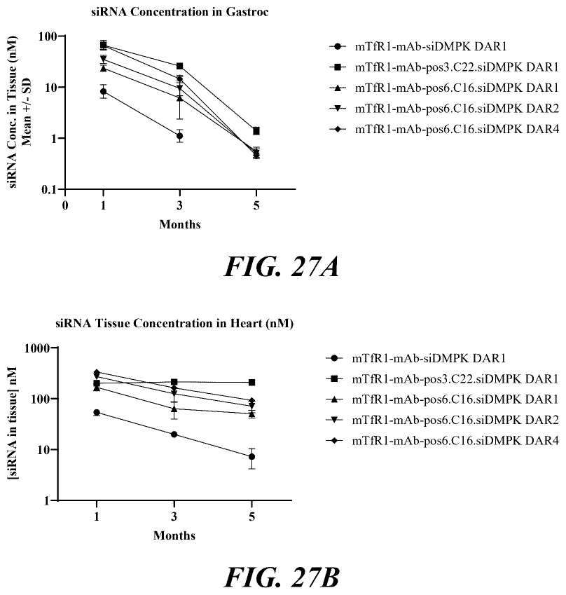

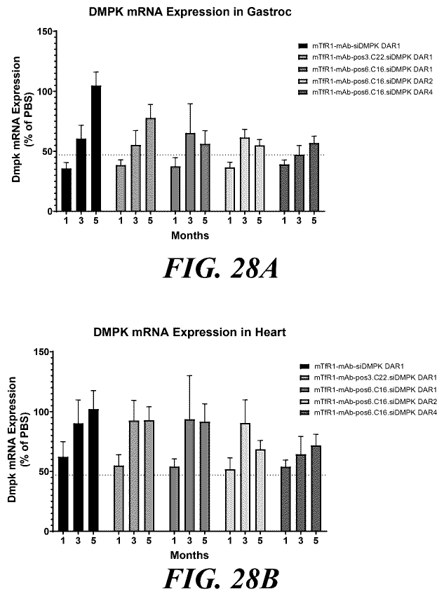

As shown in FIGS. 27A-27B and 28A-28B, DMPK siRNA tissue concentrations and DMPK mRNA expression levels were assessed with DAR1, DAR2, or DAR4 lipid-mAb AOCs with the C16 hydrocarbon chain conjugated at position 6 passenger strand or DAR1 lipid-mAb AOCs with the C22 hydrocarbon chain conjugated at position 3 passenger strand or a DAR1 non-lipid-mAb AOC at month 1, 3, or 5 in the gastroc and heart obtained from mice following a single intravenous (IV) injection of the AOCs at Day 0.

At Month 1, 3, or 5 DMPK siRNA tissue concentrations were quantified in tissue obtained from wild type mice that were administered a single intravenous injection of DAR1, DAR2, or DAR4 lipid-mAb AOCs with the C16 hydrocarbon chain conjugated at position 6 passenger strand or DAR1 lipid-mAb AOCs with the C22 hydrocarbon chain conjugated at position 3 passenger strand or a DAR1 non-lipid-mAb AOC.

As shown in FIGS. 27A-27B, all lipid mAb AOCs were able to deliver higher amount of siRNA to all muscle tissues than the non-lipid mAb AOC. The DAR1, DAR2, or DAR4 lipid-mAb AOCs with the C16 hydrocarbon chain conjugated at position 6 passenger strand or DAR1 lipid-mAb AOCs with the C22 hydrocarbon chain conjugated at position 3 passenger strand delivered similar amounts of siRNA in the heart and gastroc muscle tissues.

As shown in FIGS. 28A-28B, all lipid FabOCs were able to reduce mRNA expression levels more than non-lipid FabOC in all muscle tissues.

Overall, the lipid FabOCs with C16, 20, or 22 hydrocarbon chains conjugated to the linker have similar biological activities as the lipid FabOCs with C22 hydrocarbon chain conjugated to the passenger strand of the siRNA.

### Example 18: Dose Escalation of Human TfR1 Targeted mAb AOC DAR1 and Fab C22 siRNA Conjugates (FabOC) with Intravenous and Subcutaneous Dosing in Humanized TfR1 Mice

DMPK: A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The passenger strand was modified with 2′-O—C22 at position 3 nucleotide.

### Conjugate Synthesis and Characterization

The CD71 mAb and Fab-siRNA conjugates were made, purified and characterized as described in Examples 1-3. All conjugates were made through cysteine conjugation with a SMCC linker. Conjugates were characterized chromatographically as described in Table 53.

TABLE 53
HPLC Retention Time (RT) in Minutes

| | | RT, SAX | RT, SEC |
|---|---|---|---|
| Group(s) | Conjugate | Method-3 | Method-2 |
| 1 | hTfR1-mAb-siDMPK DAR1 | 3.141 | 13.078 |
| 2 | hTfR1-Fab-pos3.C22.siDMPK DAR1 | 3.683 | 17.287 |

### In Vivo Study Design

The conjugates were assessed for their ability to mediate mRNA downregulation of myotonic dystrophy protein kinase (DMPK) in skeletal muscle in vivo in humanized TfR1 mice. Mice were dosed via intravenous (IV) injection or subcutaneous (SC) with PBS vehicle control or the indicated doses as shown in Table 54. After 28 days, gastrocnemius (gastroc), tibialis anterior (TA), liver, kidney, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 54
In vivo study design testing dose escalation of DMPK siRNA mAb and Fab AOCs in humanized
TfR1 Mice

| | | siRNA | | | | |
|---|---|---|---|---|---|---|
| | | dose | Dose | Dose | | Harvest |
| Group | Test Article | (mg/kg) | (#) | Schedule | ROA | (Day) |
| 1 | hTfR1-mAb-siDMPK DAR1 | 1, 2, 4 | 1 | single | IV | 28 |
| 2 | hTfR1-Fab- | 0.5, 1, 2 | 1 | single | IV | 28 |
| | pos3.C22.siDMPK DAR1 | | | | | |
| 3 | hTfR1-mAb-siDMPK DAR1 | 1, 2, 4 | 1 | single | SC | 28 |
| 4 | hTfR1-Fab- | 0.5, 1, 2 | 1 | single | SC | 28 |
| | pos3.C22.siDMPK DAR1 | | | | | |
| 5 | PBS | N/A | 1 | single | IV | 28 |

TABLE 55
siRNA Sequences used in Example 18

| | | | SEQ | | SEQ |
|---|---|---|---|---|---|
| Compound | | | ID | | ID |
| No. | siRNA Ref. | siRNA Guide Sequence | NO: | siRNA Passenger Sequence | NO: |
| 1 | siDMPK | [5vp_una2moe](U)[ps].[fl2r](A) | 12 | [5am6_p_m](C)[ps].m(C)[ps].m | 48 |
| | | [ps].m(A)p.m(U)p.m(A)p.[fl2r] | | (G)p.m(A)p.m(C)p.m(A)p.[f12r] | |
| | | (C)p.m(C)p.m(G)p.m(A)p.m(G) | | (U)p.[fl2r](U)p.[fl2r](C)p.m(C) | |
| | | p.m(G)p.m(A)p.m(A)p.[fl2r](U) | | p.m(U)p.m(C)p.m(G)p.m(G)p.m( | |
| | | p.m(G)p.[fl2r](U)p.m(C)p.m(G) | | U)p.m(A)p.m(U)[ps].m(U)[ps]. | |
| | | p.m(G)[ps].m(U)[ps].m(U) | | m(A) | |
| 2 | Pos3.C22.si | [5vp_una2moe](U)[ps].[fl2r](A) | 12 | [5am6_p_m](C)[ps].m(C)[ps].[r | 81 |
| | DMPK | [ps].m(A)p.m(U)p.m(A)p.[f12r] | | c22](G)p.m(A)p.m(C)p.m(A)p.[f | |
| | | (C)p.m(C)p.m(G)p.m(A)p.m(G) | | 12r](U)p.[f12r](U)p.[fl2r](C)p.m | |
| | | p.m(G)p.m(A)p.m(A)p.[fl2r](U) | | (C)p.m(U)p.m(C)p.m(G)p.m(G) | |
| | | p.m(G)p.[fl2r](U)p.m(C)p.m(G) | | p.m(U)p.m(A)p.m(U)[ps].m(U)[p | |
| | | p.m(G)[ps].m(U)[ps].m(U) | | s].m(A) | |

5vp_una2moe 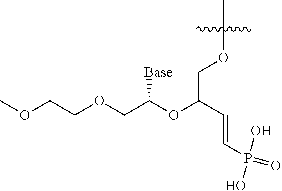
5am6_p_m 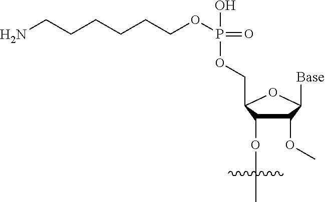
rc22

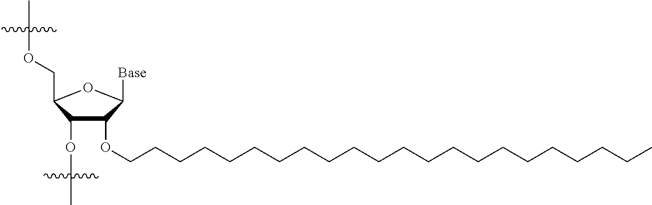

In Table 55, m indicates the 2′-O-methyl (2′-OMe) ribose modification; [fl2r] indicates the 2′-deoxy-2′-fluoro(2′—F) ribose modification; [ps] indicates phosphorothioate (PS) linkage; p indicates phosphodiester (PO) linkage.

### Results

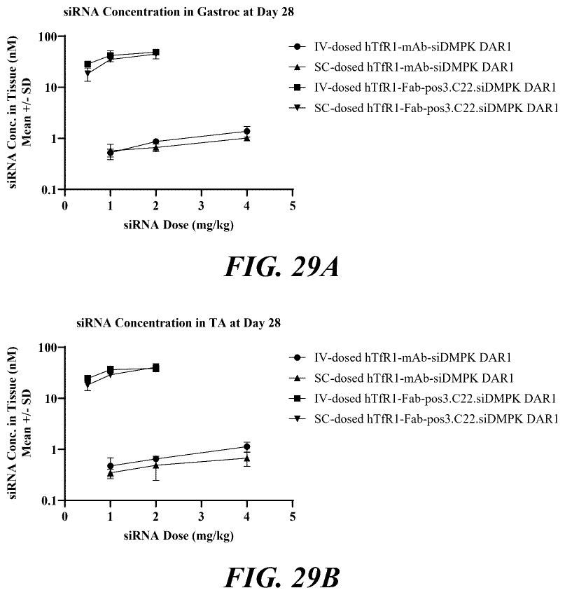

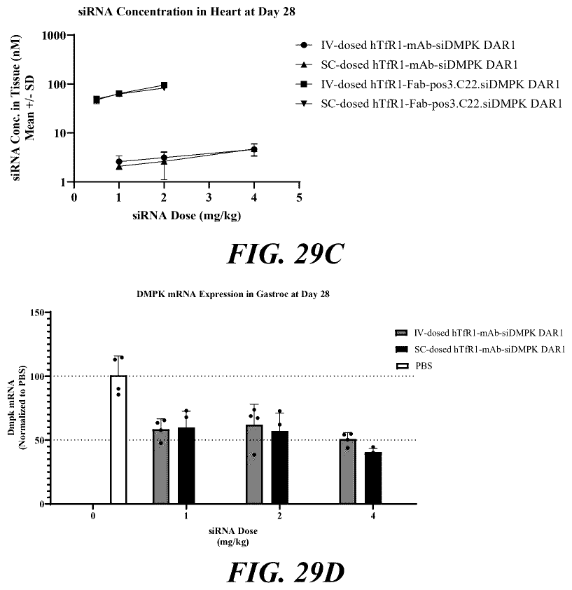

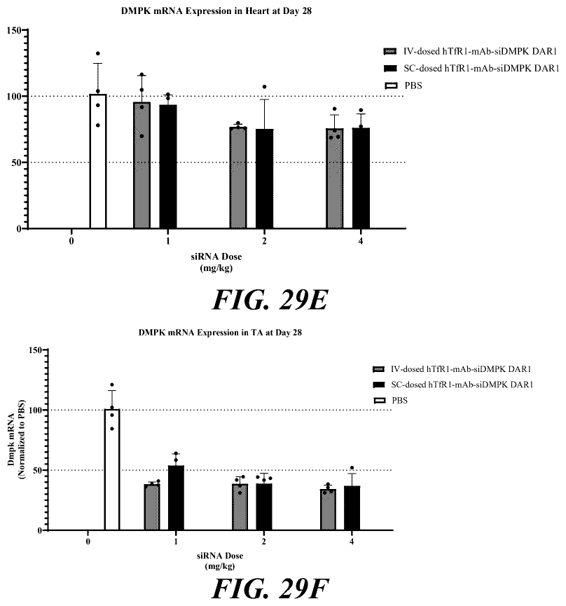

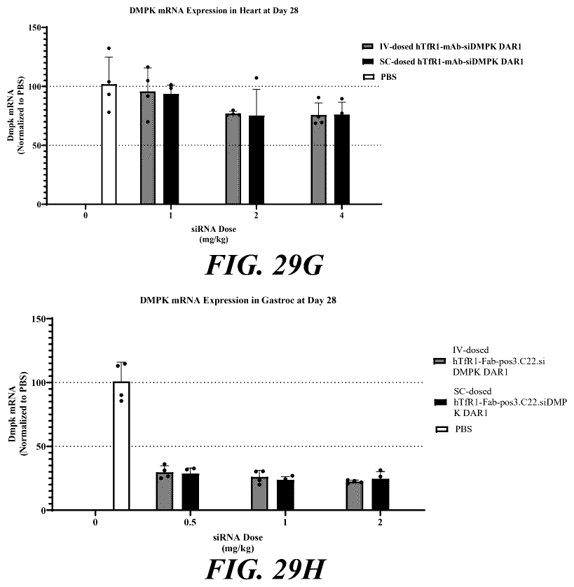

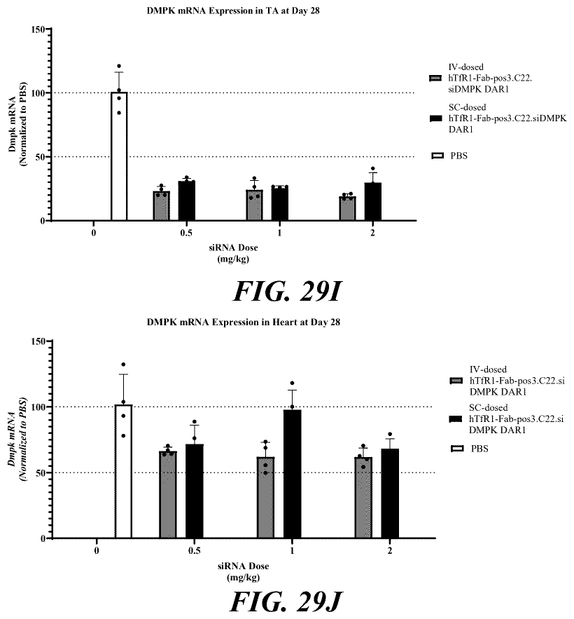

As shown in FIGS. 29A-29J, DMPK siRNA tissue concentrations and DMPK mRNA expression levels were assessed with a DAR1 lipid FabOC with the C22 hydrocarbon chain conjugated at position 3 passenger strand in the gastrocnemius (gastroc), tibialis anterior (TA), heart, kidney, and liver obtained from mice following a single intravenous (IV) or subcutaneous (SC) injection of a DAR1 control mAb AOC (hTfR1-mAb-siDMPK DAR1) or the DAR1 lipid FabOC with the C22 hydrocarbon chain conjugated at position 3 of the passenger strand (hTfR1-Fab-pos3.C22.siDMPK DAR1) of the siRNA at Day 0.

At day 28 siRNA tissue concentrations were quantified in tissue obtained from humanized mice that were administered a single intravenous or subcutaneous injection of DAR1 or lipid FabOCs with C22 hydrocarbon chain conjugated at position 3 of the passenger strand and compared to of a DAR1 control mAb AOC (hTfR1-mAb-siDMPK DAR1). The lipid-FabOC was able to deliver higher amount of siRNA to all muscle tissues than the non-lipid mAb AOC (FIGS. 29A-29C). Lipid FabOCs delivered higher levels of siRNA in cardiac tissue than the levels in skeletal muscles.

The lipid FabOCs administered intravenously and subcutaneously delivered higher siRNA tissue concentrations than the non-lipid mAb AOC whether administered subcutaneously or intravenously.

At day 28, mRNA expression levels were quantified in tissue obtained from humanized mice that were administered a administered a single intravenous or subcutaneous injection of DAR1 lipid FabOCs with C22 hydrocarbon chain conjugated at position 3 of the passenger strand and compared to of a DAR1 control mAb AOC (hTfR1-mAb-siDMPK DAR1). Lipid FabOCs administered intravenously or subcutaneously were able to decrease mRNA expression levels in skeletal muscle tissue and cardiac tissue (FIGS. 29D-29J) at to greater degree than the non-lipid mAb AOC whether administered intravenously or subcutaneously.

Overall, lipid-FabOC with the C22 hydrocarbon chain conjugated at position 3 of the passenger strand administered subcutaneously or intravenously were able to deliver high amount of siRNA to tissues and decrease mRNA expression levels in skeletal muscle tissues.

### Example 19: Duration of Action of Intravenously Administered Human TfR1 Targeted mAb DAR1 AOC and Subcutaneously Administered Fab C22 DAR1 siRNA Conjugate (FabOC) in Humanized TfR1 Mice

DMPK: A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The passenger strand of the hTfR1-Fab-pos3.C22.siDMPK DAR1 was modified with 2′-O—C22 at position 3 nucleotide.

### Conjugate Synthesis and Characterization

The CD71 mAb and Fab-siRNA conjugates were made, purified and characterized as described in Examples 1-3. All conjugates were made through cysteine conjugation with a SMCC linker. Conjugates were characterized chromatographically as described in Table 56.

TABLE 56
HPLC Retention Time (RT) in Minutes

| | | RT, SAX | RT, SEC |
|---|---|---|---|
| Group(s) | Conjugate | Method-3 | Method-2 |
| 1 | hTfR1-mAb-siDMPK DAR1 | 3.141 | 13.078 |
| 2 | hTfR1-Fab-pos3.C22.siDMPK DAR1 | 3.683 | 17.287 |

### In Vivo Study Design

The conjugates were assessed for their ability to mediate mRNA downregulation of myotonic dystrophy protein kinase (DMPK) in skeletal muscle in vivo in humanized TfR1 mice. Mice were dosed via intravenous (IV) injection or subcutaneous (SC) with PBS vehicle control or the indicated doses as shown in Table 57. After Month 1, 3, 6, and 9 gastrocnemius (gastroc), tibialis anterior (TA), liver, kidney, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 57
In vivo study design testing dose escalation of DMPK siRNA mAb and Fab AOCs in humanized
TfR1 Mice

| | | siRNA | | | | |
|---|---|---|---|---|---|---|
| | | dose | Dose | Dose | | Harvest |
| Group | Test Article | (mg/kg) | (#) | Schedule | ROA | (Day) |
| 1 | hTfR1-mAb-siDMPK DAR1 | 4 | 1 | single | IV | 1, 3, 6 |
| 2 | hTfR1-Fab- | 2 | 1 | single | SC | 1, 3, 6, 9 |
| | pos3.C22.siDMPK DAR1 | | | | | |
| 5 | PBS | N/A | 1 | single | IV | 1, 3, 6, 9 |

TABLE 58
siRNA Sequences used in Example 18

| | | | SEQ | | SEQ |
|---|---|---|---|---|---|
| Compound | SiRNA | | ID | | ID |
| No. | Ref. | siRNA Guide Sequence | NO: | siRNA Passenger Sequence | NO: |
| 1 | siDMPK | [5vp_una2moe](U)[ps].[fl2r](A) | 12 | [5am6_p_m](C)[ps].m(C)[ps].m | 48 |
| | | [ps].m(A)p.m(U)p.m(A)p.[fl2r] | | (G)p.m(A)p.m(C)p.m(A)p.[f12r] | |
| | | (C)p.m(C)p.m(G)p.m(A)p.m(G) | | (U)p.[f12r](U)p.[f12r](C)p.m(C)p. | |
| | | p.m(G)p.m(A)p.m(A)p.[fl2r](U)p. | | m(U)p.m(C)p.m(G)p.m(G)p.m | |
| | | m(G)p.[fl2r](U)p.m(C)p.m(G)p. | | (U)p.m(A)p.m(U)[ps].m(U)[ps]. | |
| | | m(G)[ps].m(U)[ps].m(U) | | m(A) | |
| 2 | Pos3.C22. | [5vp_una2moe](U)[ps].[f12r](A) | 12 | [5am6_p_m](C)[ps].m(C)[ps]. [r | 81 |
| | siDMPK | [ps].m(A)p.m(U)p.m(A)p.[f12r] | | c22](G)p.m(A)p.m(C)p.m(A)p.[f | |
| | | (C)p.m(C)p.m(G)p.m(A)p.m(G) | | 12r](U)p.[f12r](U)p.[f12r](C)p.m | |
| | | p.m(G)p.m(A)p.m(A)p.[fl2r](U) | | (C)p.m(U)p.m(C)p.m(G)p.m(G)p.m | |
| | | p.m(G)p.[fl2r](U)p.m(C)p.m(G) | | (U)p.m(A)p.m(U)[ps].m(U)[p | |
| | | p.m(G)[ps].m(U)[ps].m(U) | | s].m(A) | |

5vp_una2moe 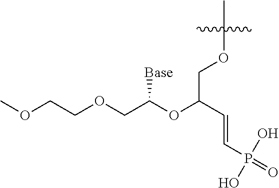
5am6_p_m 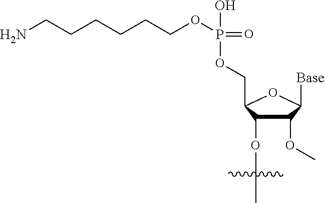
rc22

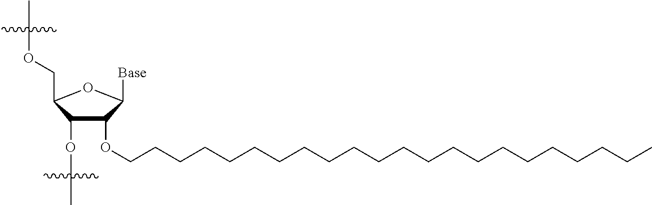

In Table 58, m indicates the 2′-O-methyl (2′-OMe) ribose modification; [fl2r] indicates the 2′-deoxy-2′-fluoro(2′—F) ribose modification; [ps] indicates phosphorothioate (PS) linkage; p indicates phosphodiester (PO) linkage.

### Results

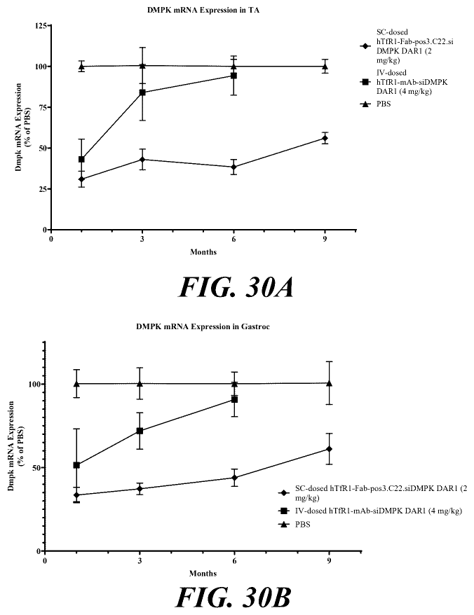

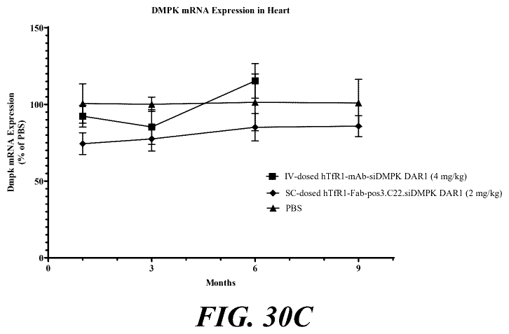

As shown in FIGS. 30A-30C, DMPK mRNA expression levels were assessed with a DAR1 lipid FabOC with the C22 hydrocarbon chain conjugated at position 3 passenger strand in the gastrocnemius (gastroc), tibialis anterior (TA), heart, kidney, and liver obtained from mice following a single intravenous (IV) or subcutaneous (SC) injection of a DAR1 control mAb AOC (hTfR1-mAb-siDMPK DAR1) at Day 0.

At Months 1, 3, 6, and 9, mRNA expression levels were quantified in tissue obtained from humanized mice that were administered that were administered a single subcutaneous injection of DAR1 lipid FabOC with C22 hydrocarbon chain conjugated at position 3 of the passenger strand and compared to a single intravenous injection of a DAR1 mAb AOC (hTfR1-mAb-siDMPK DAR1). Lipid FabOC administered subcutaneously was able to decrease mRNA expression levels in skeletal muscle tissue and cardiac tissue (FIGS. 30A-30C) to greater degree than the non-lipid mAb AOC administered intravenously.

Overall, lipid-FabOC with the C22 hydrocarbon chain conjugated at position 3 of the passenger strand administered subcutaneously was able to decrease mRNA expression levels in skeletal muscle tissues to a greater extent than non-lipid mAb AOC administered intravenously.

### Example 20: Evaluation of Lipid Fab DAR1 siRNA Conjugates (FabOCs) in Humanized TfR1 Mice at Day 28

DMPK: A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The passenger strand was modified with 2′-O—C18 at position 2 nucleotide, 2′-O—C18 at position 3 nucleotide, 2′-O—C20 at position 2 nucleotide, 2′-O—C20 at position 3 nucleotide, 2′-O—C22 at position 2 nucleotide, or 2′-O—C22 at position 3 nucleotide.

### Conjugate Synthesis and Characterization

The CD71 mAb and Fab-siRNA conjugates were made, purified and characterized as described in Examples 1-3. All conjugates were made through cysteine conjugation with a SMCC linker. Conjugates were characterized chromatographically as described in Table 59.

TABLE 59
HPLC Retention Time (RT) in Minutes

| | | RT, SAX | RT, SEC |
|---|---|---|---|
| Group(s) | Conjugate | Method-3 | Method-2 |
| 1 | hTfR1-Fab-siDMPK DAR1 | 2.993 | 15.733 |
| 2 | hTfR1-Fab-siDMPK DAR2 | 3.400 | 14.385 |
| 3 | hTfR1-Fab-pos2.C18.siDMPK DAR1 | 3.061 | 16.291 |
| 4 | hTfR1-Fab-pos2.C18.siDMPK DAR2 | 3.465 | 14.699 |
| 5 | hTfR1-Fab-pos3.C18.siDMPK DAR1 | 3.073 | 16.585 |
| 6 | hTfR1-Fab-pos3.C18.siDMPK DAR2 | 3.485 | 14.91 |
| 7 | hTfR1-Fab-pos2.C20.siDMPK DAR1 | 3.073 | 16.42 |
| 8 | hTfR1-Fab-pos2.C20.siDMPK DAR2 | 3.489 | 14.697 |
| 9 | hTfR1-Fab-pos3.C20.siDMPK DAR1 | 3.093 | 16.946 |
| 10 | hTfR1-Fab-pos3.C20.siDMPK DAR2 | 3.522 | 14.872 |
| 11 | hTfR1-Fab-pos2.C22.siDMPK DAR1 | 3.125 | 16.684 |
| 12 | hTfR1-Fab-pos2.C22.siDMPK DAR2 | 3.495 | 14.697 |
| 13 | hTfR1-Fab-pos3.C22.siDMPK DAR1 | 3.141 | 17.393 |
| 14 | hTfR1-Fab-pos3.C22.siDMPK DAR2 | 3.523 | 14.83 |

### In Vivo Study Design

The conjugates were assessed for their ability to mediate mRNA downregulation of myotonic dystrophy protein kinase (DMPK) in skeletal muscle in vivo in humanized TfR1 mice. Mice were dosed via intravenous (IV) injection or subcutaneous (SC) with PBS vehicle control or the indicated doses as shown in Table 60. After 28 days, gastrocnemius (gastroc), tibialis anterior (TA), liver, kidney, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt)

TABLE 60
In vivo study design testing dose escalation of DMPK siRNA mAb and Fab AOCs in
humanized TfR1 Mice

| | | SiRNA | | |
|---|---|---|---|---|
| | | dose | Doses | Harvest |
| Group | Test Article | (mg/kg) | (#) | (Day) |
| 1 | hTfR1-Fab-siDMPK DAR1 | 1 | 1 | 28 |
| 2 | hTfR1-Fab-siDMPK DAR2 | 2 | 1 | 28 |
| 3 | hTfR1-Fab-pos2.C18.siDMPK DAR1 | 1 | 1 | 28 |
| 4 | hTfR1-Fab-pos2.C18.siDMPK DAR2 | 2 | 1 | 28 |
| 5 | hTfR1-Fab-pos3.C18.siDMPK DAR1 | 1 | 1 | 28 |
| 6 | hTfR1-Fab-pos3.C18.siDMPK DAR2 | 2 | 1 | 28 |
| 7 | hTfR1-Fab-pos2.C20.siDMPK DAR1 | 1 | 1 | 28 |
| 8 | hTfR1-Fab-pos2.C20.siDMPK DAR2 | 2 | 1 | 28 |
| 9 | hTfR1-Fab-pos3.C20.siDMPK DAR1 | 1 | 1 | 28 |
| 10 | hTfR1-Fab-pos3.C20.siDMPK DAR2 | 2 | 1 | 28 |
| 11 | hTfR1-Fab-pos2.C22.siDMPK DAR1 | 1 | 1 | 28 |
| 12 | hTfR1-Fab-pos2.C22.siDMPK DAR2 | 2 | 1 | 28 |
| 13 | hTfR1-Fab-pos3.C22.siDMPK DAR1 | 1 | 1 | 28 |
| 14 | hTfR1-Fab-pos3.C22.siDMPK DAR2 | 2 | 1 | 28 |
| 15 | PBS | N/A | N/A | 28 |

TABLE 61
siRNA Sequences used in Example 18

| | | | SEQ | | SEQ |
|---|---|---|---|---|---|
| Compound | siRNA | | ID | | ID |
| No. | Ref. | siRNA Guide Sequence | NO: | siRNA Passenger Sequence | NO: |
| 1 | SiDMPK | [5vp_una2moe](U)[ps].[f12r](A)[ps]. | 12 | [5am6_p_m](C)[ps].m(C)[ps].m(G) | 48 |
| | | m(A)p.m(U)p.m(A)p.[fl2r](C)p.m(C) | | p.m(A)p.m(C)p.m(A)p.[f12r](U)p. | |
| | | p.m(G)p.m(A)p.m(G)p.m(G)p.m(A)p. | | [f12r](U)p.[fl2r](C)p.m(C)p.m(U) | |
| | | m(A)p.[fl2r](U)p.m(G)p.[fl2r](U)p.m | | p.m(C)p.m(G)p.m(G)p.m(U)p.m(A) | |
| | | (C)p.m(G)p.m(G)[ps].m(U)[ps].m(U) | | p.m(U)[ps].m(U)[ps].m(A) | |
| 2 | Pos2.C18. | [5vp_una2moe](U)[ps].[f12r](A)[ps]. | 12 | [5am6_p_m](C)[ps]. | 82 |
| | siDMPK | m(A)p.m(U)p.m(A)p.[fl2r](C)p.m(C) | | [rc18](C)[ps].m(G)p.m(A)p.m(C) | |
| | | p.m(G)p.m(A)p.m(G)p.m(G)p.m(A)p. | | p.m(A)p.[f12r](U)p.[f12r](U)p.[fl2r] | |
| | | m(A)p.[fl2r](U)p.m(G)p.[fl2r](U)p.m | | (C)p.m(C)p.m(U)p.m(C)p.m(G)p.m | |
| | | (C)p.m(G)p.m(G)[ps].m(U)[ps].m(U) | | (G)p.m(U)p.m(A)p.m(U)[ps].m(U) | |
| | | | | [ps].m(A) | |
| 3 | Pos3.C18. | [5vp_una2moe](U)[ps].[f12r](A)[ps]. | 12 | [5am6_p_m](C)[ps].m(C)[ps]. [rc1 | 83 |
| | siDMPK | m(A)p.m(U)p.m(A)p.[fl2r](C)p.m(C) | | 8](G)p.m(A)p.m(C)p.m(A)p.[f12r] | |
| | | p.m(G)p.m(A)p.m(G)p.m(G)p.m(A)p. | | (U)p.[f12r](U)p.[f12r](C)p.m(C)p.m | |
| | | m(A)p.[fl2r](U)p.m(G)p.[fl2r](U)p.m | | (U)p.m(C)p.m(G)p.m(G)p.m(U)p. | |
| | | (C)p.m(G)p.m(G)[ps].m(U)[ps].m(U) | | m(A)p.m(U)[ps].m(U)[ps].m(A) | |
| 4 | Pos2.C20. | [5vp_una2moe](U)[ps].[fl2r](A)[ps]. | 12 | [5am6_p_m](C)[ps]. | 84 |
| | siDMPK | m(A)p.m(U)p.m(A)p.[fl2r](C)p.m(C) | | [rc20](C)[ps].m(G)p.m(A)p.m(C) | |
| | | p.m(G)p.m(A)p.m(G)p.m(G)p.m(A)p. | | p.m(A)p.[fl2r](U)p.[fl2r](U)p.[f12r] | |
| | | m(A)p.[fl2r](U)p.m(G)p.[fl2r](U)p.m | | (C)p.m(C)p.m(U)p.m(C)p.m(G)p.m | |
| | | (C)p.m(G)p.m(G)[ps].m(U)[ps].m(U) | | (G)p.m(U)p.m(A)p.m(U)[ps].m(U) | |
| | | | | [ps].m(A) | |
| 5 | Pos3.C20. | [5vp_una2moe](U)[ps].[f12r](A)[ps]. | 12 | [5am6_p_m](C)[ps].m(C)[ps]. [rc2 | 85 |
| | siDMPK | m(A)p.m(U)p.m(A)p.[fl2r](C)p.m(C) | | 0](G)p.m(A)p.m(C)p.m(A)p.[f12r] | |
| | | p.m(G)p.m(A)p.m(G)p.m(G)p.m(A)p. | | (U)p.[fl2r](U)p.[fl2r](C)p.m(C)p.m | |
| | | m(A)p.[fl2r](U)p.m(G)p.[fl2r](U)p.m | | (U)p.m(C)p.m(G)p.m(G)p.m(U)p. | |
| | | (C)p.m(G)p.m(G)[ps].m(U)[ps].m(U) | | m(A)p.m(U)[ps].m(U)[ps].m(A) | |
| 6 | Pos2.C22. | [5vp_una2moe](U)[ps].[fl2r](A)[ps]. | 12 | [5am6_p_m](C)[ps]. | 86 |
| | siDMPK | m(A)p.m(U)p.m(A)p.[fl2r](C)p.m(C) | | [rc22](C)[ps].m(G)p.m(A)p.m(C) | |
| | | p.m(G)p.m(A)p.m(G)p.m(G)p.m(A)p. | | p.m(A)p.[fl2r](U)p.[fl2r](U)p.[f12r] | |
| | | m(A)p.[fl2r](U)p.m(G)p.[fl2r](U)p.m | | (C)p.m(C)p.m(U)p.m(C)p.m(G)p.m | |
| | | (C)p.m(G)p.m(G)[ps].m(U)[ps].m(U) | | (G)p.m(U)p.m(A)p.m(U)[ps].m(U) | |
| | | | | [ps].m(A) | |
| 7 | Pos3.C22. | [5vp_una2moe](U)[ps].[f12r](A)[ps]. | 12 | [5am6_p_m](C)[ps].m(C)[ps]. [rc2 | 81 |
| | siDMPK | m(A)p.m(U)p.m(A)p.[fl2r](C)p.m(C) | | 2](G)p.m(A)p.m(C)p.m(A)p.[f12r] | |
| | | p.m(G)p.m(A)p.m(G)p.m(G)p.m(A)p. | | (U)p.[fl2r](U)p.[fl2r](C)p.m(C)p.m | |
| | | m(A)p.[fl2r](U)p.m(G)p.[f12r](U)p.m | | (U)p.m(C)p.m(G)p.m(G)p.m(U)p. | |
| | | (C)p.m(G)p.m(G)[ps].m(U)[ps].m(U) | | m(A)p.m(U)[ps].m(U)[ps].m(A) | |

5vp_una2moe 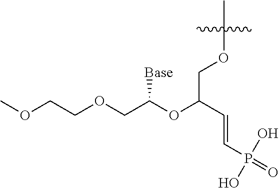
5am6_p_m 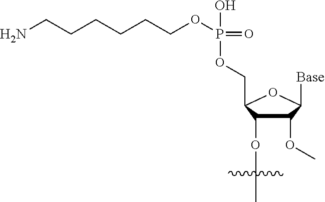
Rc18 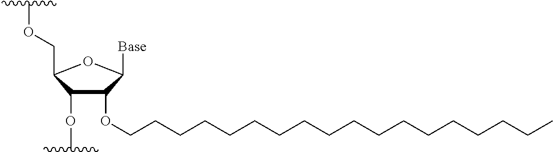
Rc20 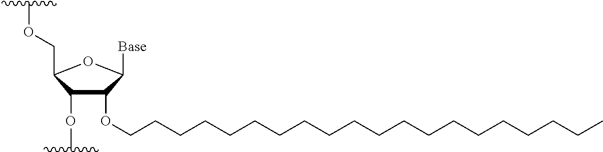
rc22

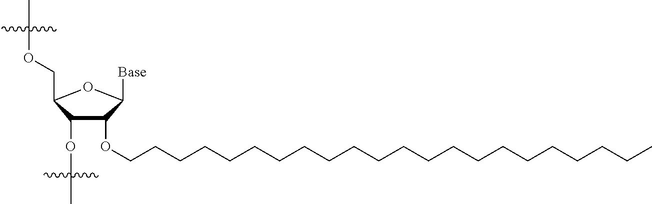

In Table 61, m indicates the 2′-O-methyl (2′-OMe) ribose modification; [fl2r] indicates the 2′-deoxy-2′-fluoro(2′—F) ribose modification; [ps] indicates phosphorothioate (PS) linkage; p indicates phosphodiester (PO) linkage.

### Results

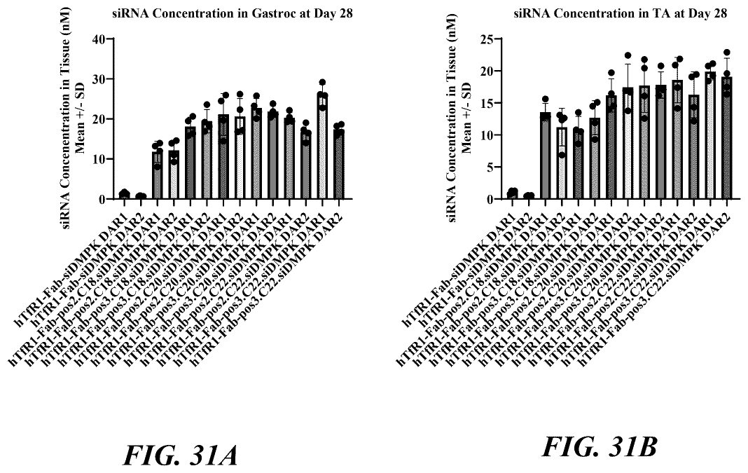

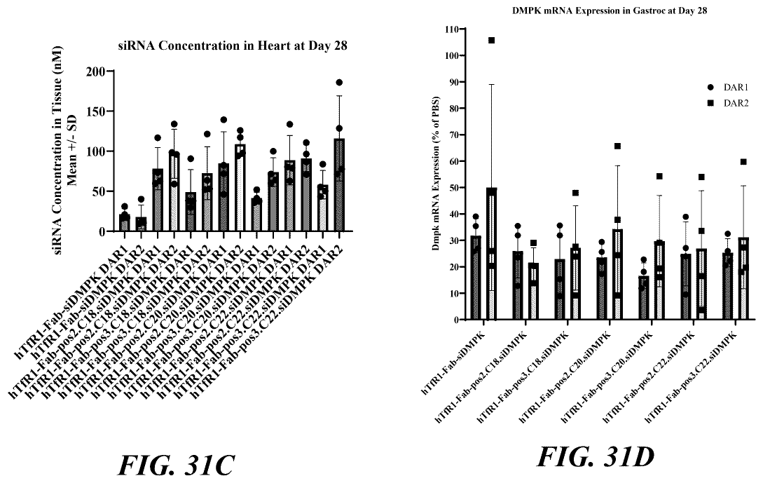

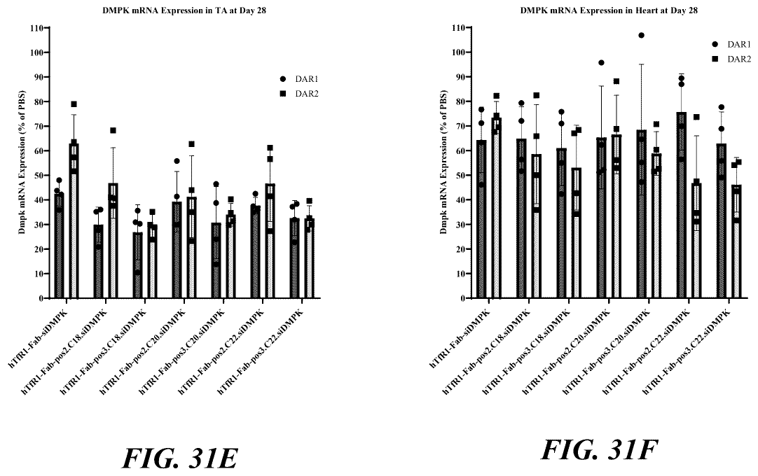

As shown in FIGS. 31A-31F, DMPK siRNA tissue concentrations and DMPK mRNA expression levels were assessed with DAR1 and DAR2 lipid FabOCs with the C22 hydrocarbon chain conjugated at position 3 or position 2 of the passenger strand, DAR1 and DAR2 lipid FabOCs with the C20 hydrocarbon chain conjugated at position 3 or position 2 of the passenger strand, DAR1 and DAR2 lipid FabOCs with the C18 hydrocarbon chain conjugated at position 3 or position 2 of the passenger strand, or DAR1 and DAR2 non-lipid hTfR1-mAb-siDMPK in the gastrocnemius (gastroc), tibialis anterior (TA), heart, kidney, and liver obtained from mice following a single intravenous (IV) injection at Day 0.

At day 28, siRNA tissue concentrations were quantified in tissue obtained from humanized mice that were administered a single intravenous injection of DAR1 and DAR2 lipid FabOCs with the C22 hydrocarbon chain conjugated at position 3 or position 2 of the passenger strand, DAR1 and DAR2 lipid FabOCs with the C20 hydrocarbon chain conjugated at position 3 or position 2 of the passenger strand, DAR1 and DAR2 lipid FabOCs with the C18 hydrocarbon chain conjugated at position 3 or position 2 of the passenger strand, or DAR1 and DAR2 non-lipid hTfR1-mAb-siDMPK. The lipid-FabOCs were able to deliver higher amount of siRNA to all muscle tissues than the non-lipid mAb AOC (FIGS. 31A-31C). Lipid FabOCs delivered higher levels of siRNA in cardiac tissue than the levels in skeletal muscles.

At day 28, mRNA expression levels were quantified in tissue obtained from humanized mice that were administered a single intravenous injection of DAR1 and DAR2 lipid FabOCs with the C22 hydrocarbon chain conjugated at position 3 or position 2 of the passenger strand, DAR1 and DAR2 lipid FabOCs with the C20 hydrocarbon chain conjugated at position 3 or position 2 of the passenger strand, DAR1 and DAR2 lipid FabOCs with the C18 hydrocarbon chain conjugated at position 3 or position 2 of the passenger strand, or DAR1 and DAR2 non-lipid hTfR1-mAb-siDMPK. Lipid FabOCs administered intravenously or subcutaneously were able to decrease mRNA expression levels in skeletal muscle tissue and cardiac tissue (FIGS. 31D-31F) to a greater degree than the DAR1 and DAR2 non-lipid FabOCs.

Overall, lipid-FabOC with the DAR1 and DAR2 lipid FabOCs with the C22 hydrocarbon chain conjugated at position 3 or position 2 of the passenger strand, DAR1 and DAR2 lipid FabOCs with the C20 hydrocarbon chain conjugated at position 3 or position 2 of the passenger strand, and DAR1 and DAR2 lipid FabOCs with the C18 hydrocarbon chain conjugated at position 3 or position 2 of the passenger strand were able to deliver higher amount of siRNA to tissues and decrease mRNA expression levels in skeletal muscle and cardiac tissues to a greater extent than DAR1 and DAR2 non-lipid hTfR1-mAb-siDMPK.

### Example 21: Duration of Action Evaluation of Lipid Fab siRNA Conjugates (FabOCs) in Humanized TfR1 Mice

DMPK: A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The passenger strand of the lipid Fab-OCs was modified with 2′-O—C18 at position 3 nucleotide, 2′-O—C20 at position 3 nucleotide, 2′-O—C22 at position 2 nucleotide, or 2′-O—C22 at position 3 nucleotide.

### Conjugate Synthesis and Characterization

The CD71 mAb and Fab-siRNA conjugates were made, purified and characterized as described in Examples 1-3. All conjugates were made through cysteine conjugation with a SMCC linker. Conjugates were characterized chromatographically as described in Table 62.

TABLE 62
HPLC Retention Time (RT) in Minutes

| | | RT, SAX | RT, SEC |
|---|---|---|---|
| Group(s | Conjugate | Method-3 | Method-2 |
| 1 | hTfR1-mAb-siDMPK DAR1 | 3.203 | 3.217 |
| 2 | hTfR1-Fab-siDMPK DAR1 | 3.564 | 3.589 |
| 3 | hTfR1-Fab-pos3.C18.siDMPK DAR1 | 3.652 | 3.718 |
| 4 | hTfR1-Fab-pos3.C20.siDMPK DAR1 | 3.676 | 3.77 |
| 5 | hTfR1-Fab-pos2.C22.siDMPK DAR1 | 3.699 | 3.779 |
| 6 | hTfR1-Fab-pos3.C22.siDMPK DAR1 | 3.714 | 3.838 |
| 7 | hTfR1-Fab-pos6.C16.siDMPK DAR1 | 3.638 | 3.714 |
| 8 | hTfR1-Fab-pos6.C16.siDMPK DAR2 | 4.113 | 3.419 |
| 9 | hTfR1-Fab-pos6.C18.siDMPK DAR1 | 3.668 | 3.818 |
| 10 | hTfR1-Fab-pos6.C18.siDMPK DAR2 | 4.146 | 3.476 |

### In Vivo Study Design

The conjugates were assessed for their ability to mediate mRNA downregulation of myotonic dystrophy protein kinase (DMPK) in skeletal muscle in vivo in humanized TfR2 mice. Mice were dosed via intravenous (IV) injection with PBS vehicle control or the indicated doses as shown in Table 62. After 28 days, gastrocnemius (gastroc), tibialis anterior (TA), liver, kidney, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 63
In vivo study design testing duration of action of DMPK siRNA mAb and Fab AOCs in
humanized TfR1 Mice

| | | siRNA | | | | |
|---|---|---|---|---|---|---|
| | | dose | Doses | Dose | | Harvest |
| Group | Test Article | (mg/kg) | (#) | Schedule | ROA | (Month) |
| 1 | hTfR1-mAb-siDMPK DAR1 | 1 | 1 | single | IV | 2, 4 |
| 2 | hTfR1-Fab-siDMPK DAR1 | 1 | 1 | single | IV | 2, 4 |
| 3 | hTfR1-Fab- | 1 | 1 | single | IV | 2, 4 |
| | pos3.C18.siDMPK DAR1 | | | | | |
| 4 | hTfR1-Fab- | 1 | 1 | single | IV | 2, 4 |
| | pos3.C20.siDMPK DAR1 | | | | | |
| 5 | hTfR1-Fab- | 1 | 1 | single | IV | 2, 4 |
| | pos2.C22.siDMPK DAR1 | | | | | |
| 6 | hTfR1-Fab- | 1 | 1 | single | IV | 2, 4 |
| | pos3.C22.siDMPK DAR1 | | | | | |
| 7 | hTfR1-Fab- | 1 | 1 | single | IV | 2, 4 |
| | pos6.C16.siDMPK DAR1 | | | | | |
| 8 | hTfR1-Fab- | 2 | 1 | single | IV | 2, 4 |
| | pos6.C16.siDMPK DAR2 | | | | | |
| 9 | hTfR1-Fab- | 1 | 1 | single | IV | 2, 4 |
| | pos6.C18.siDMPK DAR1 | | | | | |
| 14 | hTfR1-Fab- | 2 | 1 | single | IV | 2, 4 |
| | pos6.C18.siDMPK DAR2 | | | | | |
| 15 | PBS | N/A | N/A | single | IV | 2, 4 |

TABLE 64
siRNA Sequences used in Example 21

| | | | SEQ | | SEQ |
|---|---|---|---|---|---|
| Compound | siRNA | | ID | | ID |
| No. | Ref. | siRNA Guide Sequence | NO: | siRNA Passenger Sequence | NO: |
| 1 | siDMPK | (U)[ps].[fl2r](A)[ps].m(A)p.m(U) | 16 | [5am6_p_m](C)[ps].m(C)[ps].m | 48 |
| | | p.m(A)p.[fl2r](C)p.m(C)p.m(G) | | (G)p.m(A)p.m(C)p.m(A)p.[fl2r](U) | |
| | | p.m(A)p.m(G)p.m(G)p.m(A)p.m | | p.[fl2r](U)p.[fl2r](C)p.m(C)p.m | |
| | | (A)p.[fl2r](U)p.m(G)p.[fl2r](U)p.m | | (U)p.m(C)p.m(G)p.m(G)p.m(U) | |
| | | (C)p.m(G)p.m(G)[ps].m(U)[ps]. | | p.m(A)p.m(U)[ps].m(U)[ps].m | |
| | | m(U)} | | (A) | |
| 3 | Pos3.C18.s | [5vp_una2moe](U)[ps].[fl2r](A) | 12 | [5am6_p_m](C)[ps].m(C)[ps].[rc18] | 83 |
| | IDMPK | [ps].m(A)p.m(U)p.m(A)p.[fl2r](C) | | (G)p.m(A)p.m(C)p.m(A)p.[fl2r] | |
| | | p.m(C)p.m(G)p.m(A)p.m(G)p.m | | (U)p.[fl2r](U)p.[fl2r](C)p.m(C) | |
| | | (G)p.m(A)p.m(A)p.[fl2r](U)p.m | | p.m(U)p.m(C)p.m(G)p.m(G)p.m | |
| | | (G)p.[fl2r](U)p.m(C)p.m(G)p.m | | (U)p.m(A)p.m(U)[ps].m(U)[ps]. | |
| | | (G)[ps].m(U)[ps].m(U) | | m(A) | |
| 5 | Pos3.C20.s | [5vp_una2moe](U)[ps].[fl2r](A) | 12 | [5am6_p_m](C)[ps].m(C)[ps].[r | 85 |
| | IDMPK | [ps].m(A)p.m(U)p.m(A)p.[fl2r](C) | | (G)p.m(A)p.m(C)p.m(A)p.[fc20] | |
| | | p.m(C)p.m(G)p.m(A)p.m(G)p.m | | 12r](U)p.[fl2r](U)p.[fl2r](C)p.m(C) | |
| | | (G)p.m(A)p.m(A)p.[fl2r](U)p.m | | p.m(U)p.m(C)p.m(G)p.m(G)p.m | |
| | | (G)p.[f12r](U)p.m(C)p.m(G)p.m | | (U)p.m(A)p.m(U)[ps].m(U)[ps].m | |
| | | (G)[ps].m(U)[ps].m(U) | | (A) | |
| 6 | Pos2.C22.s | [5vp_una2moe](U)[ps].[fl2r](A) | 12 | [5am6_p_m](C)[ps].[rc22] | 86 |
| | IDMPK | [ps].m(A)p.m(U)p.m(A)p.[fl2r](C) | | (C)[ps].m(G)p.m(A)p.m(C) | |
| | | p.m(C)p.m(G)p.m(A)p.m(G)p.m | | p.m(A)p.[fl2r](U)p.[fl2r](U)p.[fl2r] | |
| | | (G)p.m(A)p.m(A)p.[fl2r](U)p.m | | (C)p.m(C)p.m(U)p.m(C)p.m(G) | |
| | | (G)p.[fl2r](U)p.m(C)p.m(G)p.m | | p.m(G)p.m(U)p.m(A)p.m(U)[ps].m | |
| | | (G)[ps].m(U)[ps].m(U) | | (U)[ps].m(A) | |
| 7 | Pos3.C22.s | [5vp_una2moe](U)[ps].[fl2r](A) | 12 | [5am6_p_m](C)[ps].m(C)[ps].[r | 81 |
| | IDMPK | [ps].m(A)p.m(U)p.m(A)p.[fl2r](C) | | (G)p.m(A)p.m(C)p.m(A)p.[fc22] | |
| | | p.m(C)p.m(G)p.m(A)p.m(G)p.m | | 12r](U)p.[f12r](U)p.[fl2r](C)p.m(C) | |
| | | (G)p.m(A)p.m(A)p.[fl2r](U)p.m | | p.m(U)p.m(C)p.m(G)p.m(G)p.m | |
| | | (G)p.[fl2r](U)p.m(C)p.m(G)p.m | | (U)p.m(A)p.m(U)[ps].m(U)[ps]. | |
| | | (G)[ps].m(U)[ps].m(U) | | m(A) | |
| 8 | Pos6.C16.s | [5vp_una2moe](U)[ps].[fl2r](A) | 12 | [5am6_p_m](C)[ps].m(C)[ps].m(G) | 49 |
| | IDMPK | [ps].m(A)p.m(U)p.m(A)p.[fl2r](C) | | p.m(A)p.m(C)p.[rc16](A)p.[fl2r] | |
| | | p.m(C)p.m(G)p.m(A)p.m(G)p.m | | (U)p.[f12r](U)p.[fl2r](C)p.m(C) | |
| | | (G)p.m(A)p.m(A)p.[fl2r](U)p. | | p.m(U)p.m(C)p.m(G)p.m(G)p.m | |
| | | m(G)p.[fl2r](U)p.m(C)p.m(G)p.m | | (U)p.m(A)p.m(U)[ps].m(U)[ps].m | |
| | | (G)[ps].m(U)[ps].m(U) | | (A) | |
| 9 | Pos6.C18.s | [5vp_una2moe](U)[ps].[fl2r](A) | 12 | [5am6_p_m](C)[ps].m(C)[ps].m | 87 |
| | IDMPK | [ps].m(A)p.m(U)p.m(A)p.[fl2r]( | | (G)p.m(A)p.m(C)p.[rc18](A)p.[fl2r] | |
| | | C)p.m(C)p.m(G)p.m(A)p.m(G)p | | (U)p.[fl2r](U)p.[fl2r](C)p.m(C) | |
| | | .m(G)p.m(A)p.m(A)p.[fl2r](U)p. | | p.m(U)p.m(C)p.m(G)p.m(G)p.m | |
| | | m(G)p.[fl2r](U)p.m(C)p.m(G)p. | | (U)p.m(A)p.m(U)[ps].m(U)[ps].m | |
| | | m(G)[ps].m(U)[ps].m(U) | | (A) | |

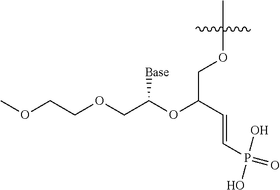

5vp_uuna2moe 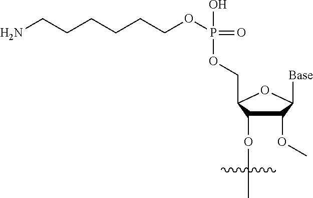
5am6_p_m 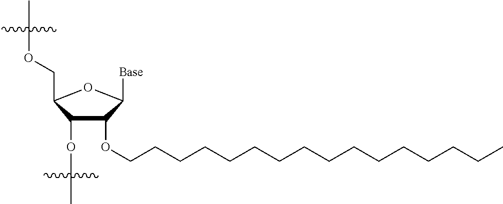
Rc16 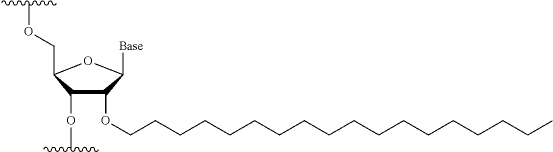
Rc18 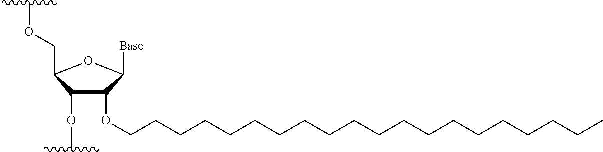
Rc20 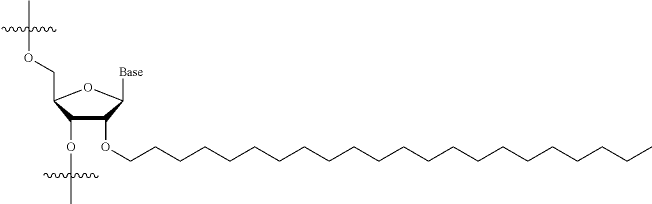
rc22

In Table 64, m indicates the 2′-O-methyl (2′-OMe) ribose modification; [fl2r] indicates the 2′-deoxy-2′-fluoro(2′—F) ribose modification; [ps] indicates phosphorothioate (PS) linkage; p indicates phosphodiester (PO) linkage.

### Results

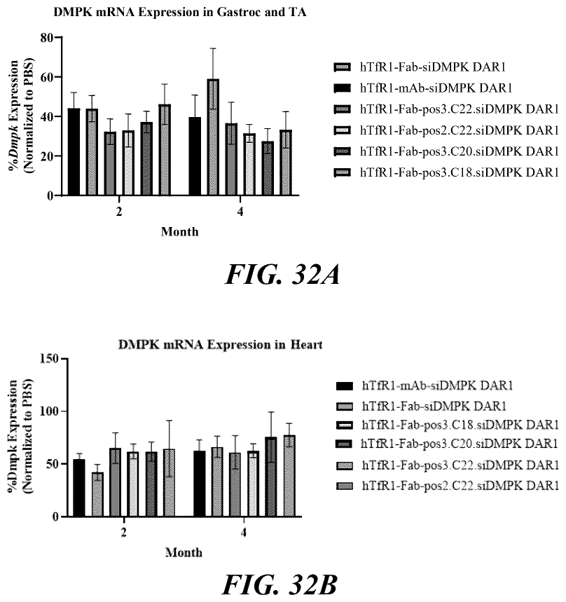

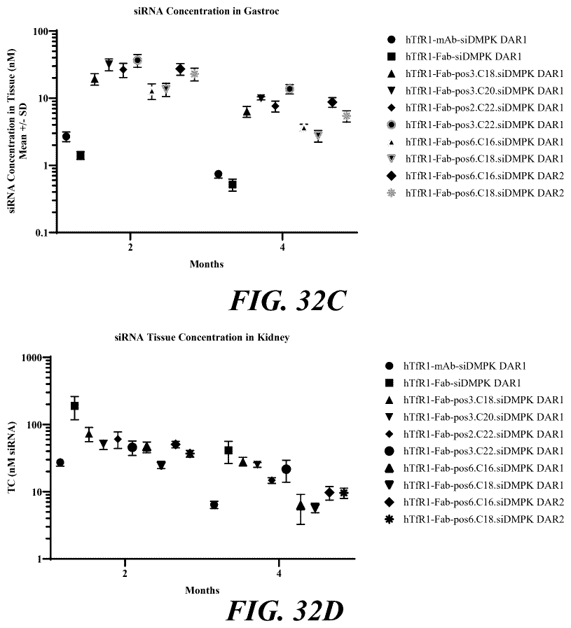

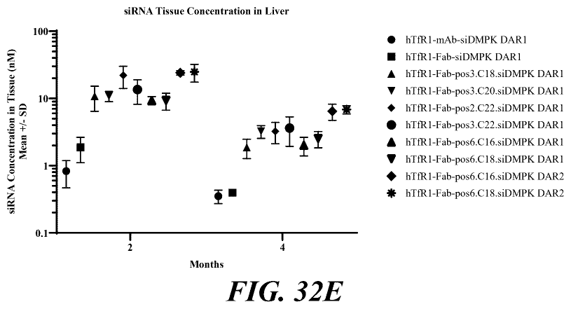

As shown in FIGS. 32A-32E, DMPK siRNA tissue concentrations and DMPK mRNA expression levels were assessed with DAR1 lipid FabOCs with the C22 hydrocarbon chain conjugated at position 3 or position 2 of the passenger strand, DAR1 lipid FabOC with the C20 hydrocarbon chain conjugated at position 3 of the passenger strand, DAR1 and DAR2 lipid FabOCs with the C18 hydrocarbon chain conjugated at position 3 or position 6 of the passenger strand, DAR1 and DAR2 lipid FabOCs with the C16 hydrocarbon chain conjugated at position 3 or position 6 of the passenger strand, DAR1 non-lipid hTfR1-mAb-siDMPK, or DAR1 non-lipid hTfR1-mAb-siDMPK in the gastrocnemius (gastroc), tibialis anterior (TA), heart, kidney, and liver obtained from mice following a single intravenous (IV) injection at Day 0.

At day 28, siRNA tissue concentrations were quantified in tissue obtained from humanized mice that were administered a single intravenous injection of DAR1 lipid FabOCs with the C22 hydrocarbon chain conjugated at position 3 or position 2 of the passenger strand, DAR1 lipid FabOC with the C20 hydrocarbon chain conjugated at position 3 of the passenger strand, DAR1 and DAR2 lipid FabOCs with the C18 hydrocarbon chain conjugated at position 3 or position 6 of the passenger strand, DAR1 and DAR2 lipid FabOCs with the C16 hydrocarbon chain conjugated at position 3 or position 6 of the passenger strand, DAR1 non-lipid hTfR1-mAb-siDMPK, or DAR1 non-lipid hTfR1-mAb-siDMPK. The lipid-FabOCs were able to deliver higher amount of siRNA to all muscle tissues than the non-lipid mAb and Fab AOCs (FIGS. 32C-32E). Lipid FabOCs delivered higher levels of siRNA in cardiac tissue than the levels in skeletal muscles.

At day 28, mRNA expression levels were quantified in tissue obtained from humanized mice that were administered a administered a single intravenous injection DAR1 lipid FabOCs with the C22 hydrocarbon chain conjugated at position 3 or position 2 of the passenger strand, DAR1 lipid FabOC with the C20 hydrocarbon chain conjugated at position 3 of the passenger strand, DAR1 and DAR2 lipid FabOCs with the C18 hydrocarbon chain conjugated at position 3 or position 6 of the passenger strand, DAR1 and DAR2 lipid FabOCs with the C16 hydrocarbon chain conjugated at position 3 or position 6 of the passenger strand, DAR1 non-lipid hTfR1-mAb-siDMPK, or DAR1 non-lipid hTfR1-mAb-siDMPK. Lipid FabOCs administered intravenously were able to decrease mRNA expression levels in skeletal muscle tissue and cardiac tissue (FIGS. 32A-32B) to a greater degree than the DAR1 non-lipid mAb and Fab AOCs.

Overall, lipid-FabOC with the single intravenous injection DAR1 lipid FabOCs with the C22 hydrocarbon chain conjugated at position 3 or position 2 of the passenger strand, DAR1 lipid FabOC with the C20 hydrocarbon chain conjugated at position 3 of the passenger strand, DAR1 and DAR2 lipid FabOCs with the C18 hydrocarbon chain conjugated at position 3 or position 6 of the passenger strand, and DAR1 and DAR2 lipid FabOCs with the C16 hydrocarbon chain conjugated at position 3 or position 6 of the passenger strand were able to deliver higher amount of siRNA to tissues and decrease mRNA expression levels in skeletal muscle and cardiac tissues to a greater extent than DAR1 non-lipid hTfR1-mAb-siDMPK or DAR1 non-lipid hTfR1-mAb-siDMPK.

### Example 22: Simultaneous Targeting of Two siRNA Targets on Same AOC with DAR2 Lipid Fab-OC Humanized TfR1 Mice

SSB: A 21-mer double-stranded siRNA with 19 bases of complementarity and 3′ dinucleotide overhangs was designed against mouse SSB. The sequence (5′ to 3′) of the guide/antisense strand was UUACAUUAAAGUCUGUUGUUU (SEQ ID NO: 5). DMPK: A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The passenger strand of the lipid Fab-OCs was modified with 2′-O—C16 at position 6 nucleotide or 2′-O—C22 at position 3 nucleotide.

### Conjugate Synthesis and Characterization

The CD71 mAb siRNA conjugates were made, purified and characterized as described below. All conjugates were made through cysteine conjugation with a SMCC linker. Conjugates were characterized chromatographically as described in Table 65.

Step 1: Addition of SMCC Linker to siRNA Duplex

SMCC was purchased from ThermoFisher (Catalog #22360) and a stock solution was prepared at 60 mg/ml in dimethyl sulfoxide (DMSO). To duplexed siRNA in deionized water was added 1 M phosphate buffer, pH 7.2 to a final concentration of 50 mM phosphate buffer and the pH adjusted to 7.6. To the duplex was added sufficient DMSO to bring the final concentration DMSO in the reaction to 60% (v/v) and 10 equivalents of the SMCC linker. The reaction was allowed to proceed for 1 hr before dilution to 5% DMSO in cold 10 mM acetate buffer, pH 6. The diluted reaction mixture was purified by spin filtration (5×) using 3K MWCO filters. The linker-modified duplex was analyzed using LCMS and stored at −20° C. until reaction with reduced antibody.

Step 2: Antibody Interchain Disulfide Reduction with TCEP and Conjugation of siRNA Duplex Targeting First of Two Targets

To a solution of antibody in phosphate buffered saline (PBS, pH 7.4) at 27.87 mg/ml protein concentration was added sufficient 0.5 M ethylenediaminetetraacetic acid (EDTA) to afford a final concentration of 2 mM EDTA in the final reaction solution. To the solution containing antibody and EDTA was added 1.5 equivalents of tris(2-carboxyethyl)phosphine hydrochloride (TCEP) in water and incubated for 4 hours at 37° C. To the resultant reduced antibody solution was added a solution of SMCC-C6-siRNA (0.9 equivalents) in 10 mM sodium acetate buffer, pH 6. The reaction proceeded for 1 hour at room temperature. Analysis of the reaction mixture by analytical SAX and SEC column chromatography showed antibody siRNA conjugate along with unreacted antibody and siRNA.

Step 3: Purification of Antibody siRNA Conjugate

The crude reaction mixture was purified by AKTA pure FPLC using anion exchange chromatography method-2. Fractions containing DAR1 and DAR2 conjugates were separated, concentrated and buffer exchanged with pH 7.4 PBS.

Step 4: Conjugation of siRNA Duplex Targeting Second of Two Targets

To the resultant DAR1 conjugate solution was added a solution of SMCC-C6-siRNA (1.1 equivalents assuming quantitative yield from step 3 of the desired DAR1 conjugate species) in 10 mM sodium acetate buffer, pH 6. The reaction proceeded for 1 hour at room temperature before quenching with 10 equivalents of N-ethyl maleimide (NEM) as a 10 mg/ml stock solution in dimethyl sulfoxide (DMSO). Analysis of the reaction mixture by analytical SAX and SEC column chromatography showed antibody siRNA conjugate along with unreacted antibody and siRNA.

Step 5: Purification of Antibody siRNA Conjugate

The crude reaction mixture was purified by AKTA pure FPLC using anion exchange chromatography method-2. Fractions containing DAR1 and DAR2 conjugates were separated, concentrated and buffer exchanged with pH 7.4 PBS.

### Step 6: Analysis of the Purified Conjugate

The isolated conjugates were characterized by SEC with SEC using size exclusion chromatography method-2 and SAX chromatography anion exchange chromatography method-3 as described in the corresponding section. All methods are described in Purification and Analytical Methods herein. Isolated conjugates had greater than 90% purity.

TABLE 65
HPLC Retention Time (RT) in Minutes

| | | RT, SAX | RT, SEC |
|---|---|---|---|
| Group(s) | Conjugate | Method-3 | Method-2 |
| 1 | hTfR1-Fab-siDMPK DAR1 | 3.406 | 3.536 |
| 2 | hTfR1-Fab-pos6.C16.siDMPK DAR1 | 3.439 | 3.625 |
| 3 | hTfR1-Fab-pos6.C16.siDMPK DAR2 | 3.925 | 3.325 |
| 4 | hTfR1-Fab-siSSB DAR1 | 3.453 | 3.544 |
| 5 | hTfR1-Fab-pos6.C16.siSSB DAR1 | 3.5 | 3.622 |
| 6 | hTfR1-Fab-pos6.C16.siSSB DAR2 | 3.958 | 3.3 |
| 7 | hTfR1-Fab-siDMPK-siSSB DAR2 | 3.856 | 3.232 |
| 8 | hTfR1-Fab-pos6.C16.siDMPK-pos6.C16.siSSB DAR2 | 3.926 | 3.327 |

### In Vivo Study Design

The conjugates were assessed for their ability to mediate mRNA downregulation of myotonic dystrophy protein kinase (DMPK) in skeletal muscle in vivo in humanized TfR1 mice. Mice were dosed via intravenous (IV) injection with PBS vehicle control or the indicated doses as shown in Table 66. After 28 days, gastrocnemius (gastroc), tibialis anterior (TA), liver, kidney, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 66
In vivo study design testing duration of action of
DMPK siRNA mAb and Fab AOCs in humanized TfR1 Mice

| | | siRNA | | |
|---|---|---|---|---|
| | | dose | Doses | Harvest |
| Group | Test Article | (mg/kg) | (#) | (Day) |
| 1 | hTfR1-Fab-siDMPK DAR1 | 0.5 | 1 | 28 |
| 2 | hTfR1-Fab-pos6.C16.siDMPK DAR1 | 0.5 | 1 | 28 |
| 3 | hTfR1-Fab-pos6.C16.siDMPK DAR2 | 1 | 1 | 28 |
| 4 | hTfR1-Fab-siSSB DAR1 | 0.5 | 1 | 28 |
| 5 | hTfR1-Fab-pos6.C16.siSSB DAR1 | 0.5 | 1 | 28 |
| 6 | hTfR1-Fab-pos6.C16.siSSB DAR2 | 1 | 1 | 28 |
| 7 | hTfR1-Fab-siDMPK-siSSB DAR2 | 1 | 1 | 28 |
| 8 | hTfR1-Fab-pos6.C16.siDMPK-pos6.C16.siSSB DAR2 | 1 | 1 | 28 |
| 15 | PBS | N/A | N/A | 28 |

* All animals used in experiments shown in Table 66 were injected with a single dose by IV administration.

TABLE 67
siRNA Sequences used in Example 22

| | | | SEQ | | SEQ |
|---|---|---|---|---|---|
| Compound | SIRNA | | ID | | ID |
| No. | Ref. | siRNA Guide Sequence | NO: | siRNA Passenger Sequence | NO: |
| 1 | siMSTN | [5vp_una2moe](U)[ps].[fl2r](U) | 14 | [5am6_p_m](G)[ps].m(G)[ps].m | 75 |
| | | [ps].m(A)p.m(U)p.m(U)p.[fl2r](A) | | (C)p.m(A)p.m(A)p.m(A)p.[fl2r](G) | |
| | | p.m(U)p.m(U)p.m(U)p.m(G)p.m | | p.[fl2r](A)p.[fl2r](A)p.m(C)p.m | |
| | | (U)p.m(U)p.m(C)p.[fl2r](U)p.m | | (A)p.m(A)p.m(A)p.m(U)p.m(A) | |
| | | (U)p.[fl2r](U)p.m(G)p.m(C)p.m | | p.m(A)p.m(U)[ps].m(A)[ps].m | |
| | | (C)[ps].m(U)[ps].m(U) | | (A) | |
| 3 | pos6.C16. | [5vp_una2moe](U)[ps].[f12r](U) | 14 | [5am6_p_m](G)[ps].m(G)[ps].m | 76 |
| | SIMSTN | [ps].m(A)p.m(U)p.m(U)p.[f12r](A) | | (C)p.m(A)p.m(A)p.[rc16](A)p.[fl2r] | |
| | | p.m(U)p.m(U)p.m(U)p.m(G)p.m | | (G)p.[fl2r](A)p.[fl2r](A)p.m(C) | |
| | | (U)p.m(U)p.m(C)p.[fl2r](U)p.m | | p.m(A)p.m(A)p.m(A)p.m(U)p.m | |
| | | (U)p.[fl2r](U)p.m(G)p.m(C)p.m | | (A)p.m(A)p.m(U)[ps].m(A)[ps].m | |
| | | (C)[ps].m(U)[ps].m(U) | | (A) | |
| 5 | siSSB | [5v_puna2moe](U)[ps].[f12r](U) | 13 | [5am6_p_m](A)[ps].m(C)[ps].m | 50 |
| | | [ps].m(A)p.m(C)p.m(A)p.[fl2r](U) | | (A)p.m(A)p.m(C)p.m(A)p.[fl2r](G) | |
| | | p.m(U)p.m(A)p.m(A)p.m(A)p.m | | p.[fl2r](A)p.[fl2r](C)p.m(U)p.m | |
| | | (G)p.m(U)p.m(C)p.[fl2r](U)p.m | | (U)p.m(U)p.m(A)p.m(A)p.m(U) | |
| | | (G)p.[fl2r](U)p.m(U)p.m(G)p.m | | p.m(G)p.m(U)[ps].m(A)[ps].m | |
| | | (U)[ps].m(U)[ps].m(U) | | (A) | |
| 6 | pos6.C16. | [5vp_una2moe](U)[ps].[fl2r](U) | 13 | [5am6_p_m](A)[ps].m(C)[ps].m | 51 |
| | siSSB | [ps].m(A)p.m(C)p.m(A)p.[fl2r](U) | | (A)p.m(A)p.m(C)p.[rc16](A)p.[fl2r] | |
| | | p.m(U)p.m(A)p.m(A)p.m(A)p.m | | (G)p.[fl2r](A)p.[fl2r](C)p.m(U) | |
| | | (G)p.m(U)p.m(C)p.[fl2r](U)p.m | | p.m(U)p.m(U)p.m(A)p.m(A)p.m | |
| | | (G)p.[fl2r](U)p.m(U)p.m(G)p.m | | (U)p.m(G)p.m(U)[ps].m(A)[ps].m | |
| | | (U)[ps].m(U)[ps].m(U) | | (A) | |

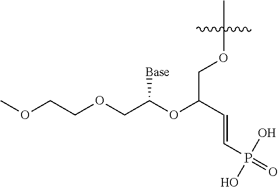

5vp_una2moe 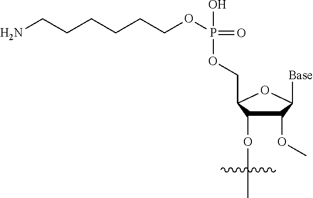
5am6_p_m 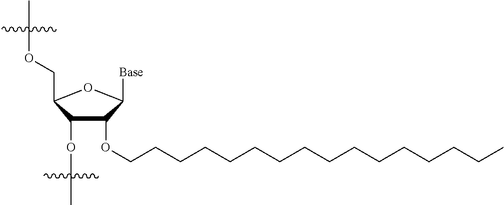
Rc16

In Table 67, m indicates the 2′-O-methyl (2′-OMe) ribose modification; [fl2r] indicates the 2′-deoxy-2′-fluoro(2′—F) ribose modification; [ps] indicates phosphorothioate (PS) linkage; p indicates phosphodiester (PO) linkage.

### Results

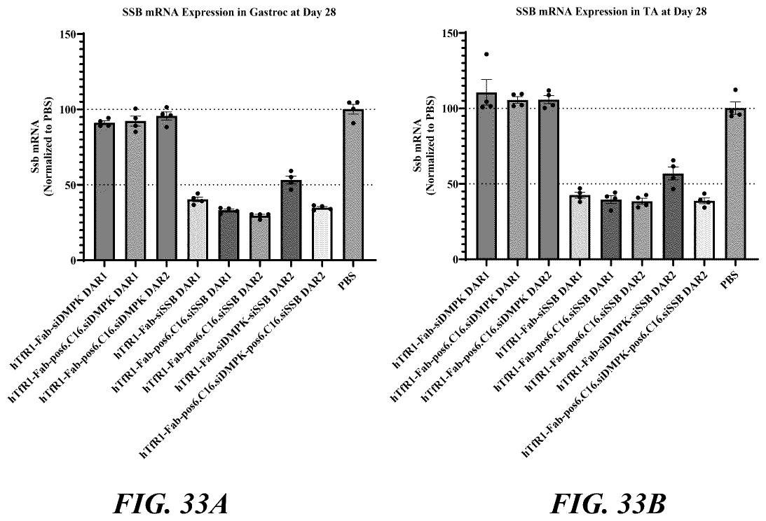

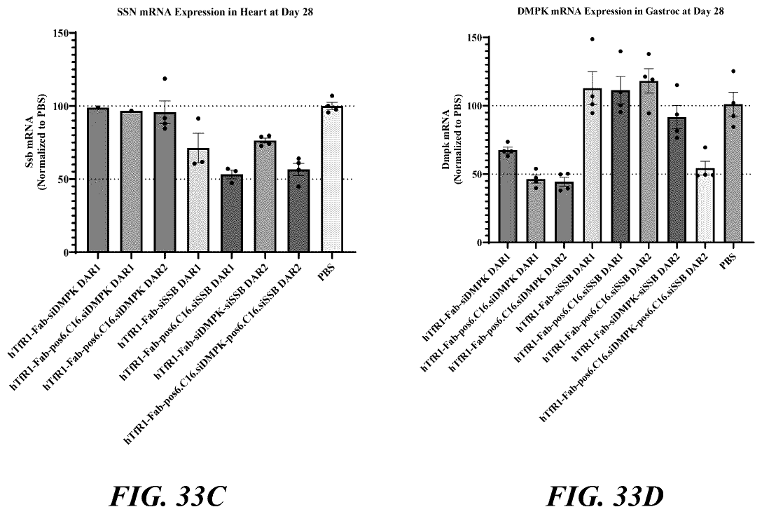

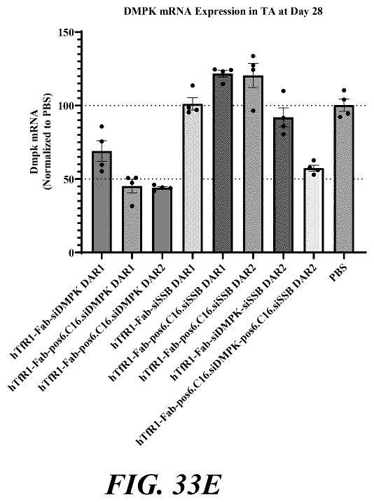

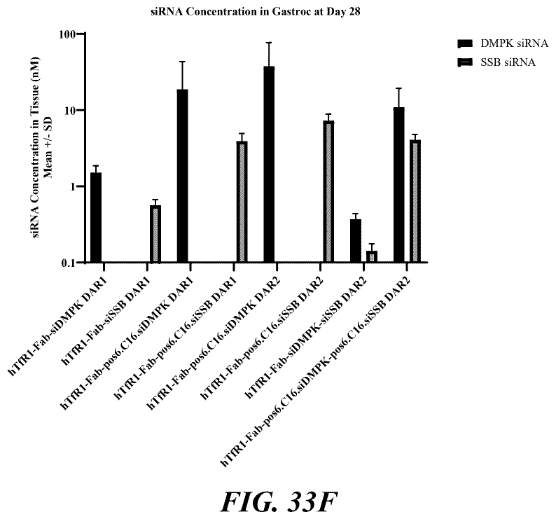

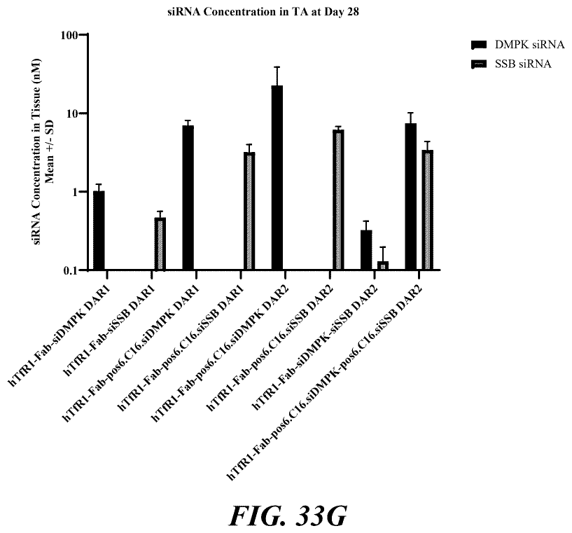

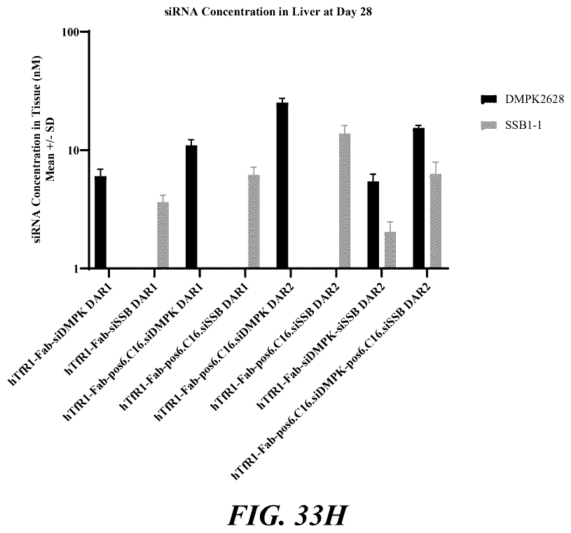

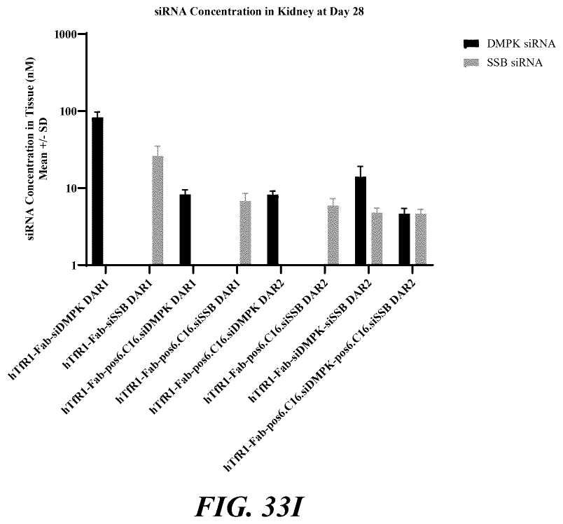

As shown in FIGS. 33A-33I, SSB and MSTN siRNA tissue concentrations and SSB and MSTN mRNA expression levels were assessed with hTfR1-Fab-siDMPK DAR1, hTfR1-Fab-pos6.C16.siDMPK DAR1, hTfR1-Fab-pos6.C16.siDMPK DAR2, hTfR1-Fab-siSSB DAR1, TfR1-Fab-pos6.C16.siSSB DAR1, hTfR1-Fab-pos6.C16.siSSB DAR2, hTfR1-Fab-siDMPK-siSSB DAR2, and hTfR1-Fab-pos6.C16.siDMPK-pos6.C16.siSSB DAR2 FabOCs in the gastrocnemius (gastroc), tibialis anterior (TA), heart, kidney, and liver obtained from mice following a single intravenous (IV) injection at Day 0.

At day 28, siRNA tissue concentrations were quantified in tissue obtained from humanized mice that were administered a single intravenous injection of hTfR1-Fab-siDMPK DAR1, hTfR1-Fab-pos6.C16.siDMPK DAR1, hTfR1-Fab-pos6.C16.siDMPK DAR2, hTfR1-Fab-siSSB DAR1, TfR1-Fab-pos6.C16.siSSB DAR1, hTfR1-Fab-pos6.C16.siSSB DAR2, hTfR1-Fab-siDMPK-siSSB DAR2, or hTfR1-Fab-pos6.C16.siDMPK-pos6.C16.siSSB DAR2 FabOCs. The lipid-FabOCs were able to deliver higher amount of siRNA to all muscle tissues than the non-lipid Fab-OCs (FIGS. 33F-33I). The DAR2 lipid-FabOCs were able to deliver higher amount of siRNA to all muscle tissues than the non-lipid DAR2 Fab-OCs (FIG. 33F-331) Lipid FabOCs delivered higher levels of siRNA in cardiac tissue than the levels in skeletal muscles.

At day 28, mRNA expression levels were quantified in tissue obtained from humanized mice that were administered a administered a single intravenous of hTfR1-Fab-siDMPK DAR1, hTfR1-Fab-pos6.C16.siDMPK DAR1, hTfR1-Fab-pos6.C16.siDMPK DAR2, hTfR1-Fab-siSSB DAR1, TfR1-Fab-pos6.C16.siSSB DAR1, hTfR1-Fab-pos6.C16.siSSB DAR2, hTfR1-Fab-siDMPK-siSSB DAR2, or hTfR1-Fab-pos6.C16.siDMPK-pos6.C16.siSSB DAR2 FabOCs. Lipid FabOCs administered intravenously were able to decrease mRNA expression levels in skeletal muscle tissue and cardiac tissue (FIG. 33A-33E) to a greater degree than the DAR1 non-lipid Fab-OCs. hTfR1-Fab-pos6.C16.siDMPK-pos6.C16.siSSB DAR2 FabOCs administered intravenously was able to decrease mRNA expression levels in skeletal muscle tissue and cardiac tissue (FIG. 33A-33E) to a greater degree than hTfR1-Fab-siDMPK-siSSB DAR2.

Overall, lipid-FabOCs with a single intravenous injection of dual-targeting SSB/MSTN hTfR1-Fab-pos6.C16.siDMPK-pos6.C16.siSSB DAR2 FabOC was able to deliver higher amount of siRNA to tissues and decrease mRNA expression levels in skeletal muscle and cardiac tissues to a greater extent than non-lipid dual-targeting SSB/MSTN hTfR1-Fab-siDMPK-siSSB DAR2.

### Example 23: Delivery of Dual-siRNA with Lipid Fab-OCs in Humanized TfR1 Mice

A 21-mer double-stranded siRNA with 19 bases of complementarity and 3′ dinucleotide overhangs was designed against mouse MSTN. The sequence (5′ to 3′) of the guide/antisense strand was UUAUUAUUUGUUCUUUGCCUU (SEQ ID NO: 4). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The passenger strand of the lipid Fab-OCs was modified with 2′-O—C16 at position 6 nucleotide or 2′-O—C22 at position 3 nucleotide.

### Conjugate Synthesis and Characterization

The CD71 mAb siRNA conjugates were made, purified and characterized as described in Examples 1-3. All conjugates were made through cysteine conjugation with a SMCC linker. Conjugates were characterized chromatographically as described in Table 68.

TABLE 68
HPLC Retention Time (RT) in Minutes

| | | RT, SAX | RT, SEC |
|---|---|---|---|
| Group(s) | Conjugate | Method-3 | Method-2 |
| 1 | hTfR1-Fab-siMSTN DAR1 | 3.39 | 3.618 |
| 2 | hTfR1-Fab-pos3.C22.siMSTN DAR1 | 3.534 | 3.945 |
| 3 | hTfR1-Fab-pos6.C16.siMSTN DAR1 | 3.453 | 3.715 |
| 4 | hTfR1-Fab-pos6.C16.siMSTN DAR2 | 3.915 | 3.405 |
| 5 | hTfR1-Fab-dual siMSTN DAR1 | 4.087 | 3.217 |
| 6 | hTfR1-Fab-dual pos3.C22.siMSTN DAR1 | 4.112 | 3.258 |
| 7 | hTfR1-Fab-dual pos6.C16.siMSTN DAR1 | 4.157 | 3.333 |
| 8 | hTfR1-Fab-dual pos6.C16x2.siMSTN DAR1 | 4.166 | 3.418 |
| 9 | hTfR1-Fab-siMSTN DAR2 | 3.858 | 3.278 |

### In Vivo Study Design

The conjugates were assessed for their ability to mediate mRNA downregulation of myostatin (MSTN) in skeletal muscle in vivo in humanized TfR1 mice. Mice were dosed via intravenous (IV) injection with PBS vehicle control or the indicated doses as shown in Table 69. After 28 days, gastrocnemius (gastroc), tibialis anterior (TA), liver, kidney, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 69
In vivo study design Delivery of Dual-siRNA with Lipid Fab-OCs in Humanized
TfR1

| | | | siRNA | | | | |
|---|---|---|---|---|---|---|---|
| | | siRNA | dose | Doses | Dose | | Harvest |
| Group | Test Article | Target | (mg/kg) | (#) | Schedule | ROA | (Day) |
| 1 | hTfR1-Fab-siMSTN DAR1 | DMPK | 0.5 | 1 | single | IV | 28 |
| 2 | hTfR1-Fab- | DMPK | 0.5 | 1 | single | IV | 28 |
| | pos3.C22.siMSTN DAR1 | | | | | | |
| 3 | hTfR1-Fab- | DMPK | 0.5 | 1 | single | IV | 28 |
| | pos6.C16.siMSTN DAR1 | | | | | | |
| 4 | hTfR1-Fab- | DMPK | 1 | 1 | single | IV | 28 |
| | pos6.C16.siMSTN DAR2 | | | | | | |
| 5 | hTfR1-Fab-dual siMSTN | DMPK | 0.5 | 1 | single | IV | 28 |
| | DAR1 | | | | | | |
| 6 | hTfR1-Fab-dual | DMPK | 0.5 | 1 | single | IV | 28 |
| | pos3.C22.siMSTN DAR1 | | | | | | |
| 7 | hTfR1-Fab-dual | DMPK | 0.5 | 1 | single | IV | 28 |
| | pos6.C16.siMSTN DAR1 | | | | | | |
| 8 | hTfR1-Fab-dual | DMPK | 0.5 | 1 | single | IV | 28 |
| | pos6.C16x2.siMSTN | | | | | | |
| | DAR1 | | | | | | |
| 9 | hTfR1-Fab-siMSTN DAR2 | N/A | 1 | N/A | single | IV | 28 |
| 15 | PBS | N/A | N/A | N/A | single | IV | 28 |

TABLE 70
siRNA Sequences used in Example 23

| | | | SEQ | | SEQ |
|---|---|---|---|---|---|
| Compound | SIRNA | | ID | | ID |
| No. | Ref. | siRNA Guide Sequence | NO: | siRNA Passenger Sequence | NO: |
| 1 | siMSTN | [5vp_una2moe](U)[ps].[fl2r](U)[ps].m | 14 | [5am6_p_m](G)[ps].m(G)[ps].m | 75 |
| | | (A)p.m(U)p.m(U)p.[f12r](A)p.m(U) | | (C)p.m(A)p.m(A)p.m(A)p.[f12r](G) | |
| | | p.m(U)p.m(U)p.m(G)p.m(U)p.m(U) | | p.[fl2r](A)p.[fl2r](A)p.m(C)p.m | |
| | | p.m(C)p.[fl2r](U)p.m(U)p.[f12r](U) | | (A)p.m(A)p.m(A)p.m(U)p.m(A) | |
| | | p.m(G)p.m(C)p.m(C)[ps].m(U)[ps].m | | p.m(A)p.m(U)[ps].m(A)[ps].m | |
| | | (U) | | (A) | |
| 3 | pos6.C16. | [5vp_una2moe](U)[ps].[fl2r](U)[ps].m | 14 | [5am6_p_m](G)[ps].m(G)[ps].m | 76 |
| | siMSTN | (A)p.m(U)p.m(U)p.[fl2r](A)p.m(U) | | (C)p.m(A)p.m(A)p.[rc16](A)p.[f12r] | |
| | | p.m(U)p.m(U)p.m(G)p.m(U)p.m(U) | | (G)p.[fl2r](A)p.[fl2r](A)p.m(C) | |
| | | p.m(C)p.[f12r](U)p.m(U)p.[f12r](U) | | p.m(A)p.m(A)p.m(A)p.m(U)p.m | |
| | | p.m(G)p.m(C)p.m(C)[ps].m(U)[ps].m | | (A)p.m(A)p.m(U)[ps].m(A)[ps].m | |
| | | (U) | | (A) | |
| 5 | Pos3.C22. | [5vp_una2moe](U)[ps].[f12r](U)[ps].m | 14 | [5am6_p_m](G)[ps].m(G)[ps].[rc22] | 88 |
| | siMSTN | (A)p.m(U)p.m(U)p.[fl2r](A)p.m(U) | | (C)p.m(A)p.m(A)p.m(A)p.[fl2r] | |
| | | p.m(U)p.m(U)p.m(G)p.m(U)p.m(U) | | (G)p.[fl2r](A)p.[fl2r](A)p.m | |
| | | p.m(C)p.[fl2r](U)p.m(U)p.[fl2r](U) | | (C)p.m(A)p.m(A)p.m(A)p.m(U) | |
| | | p.m(G)p.m(C)p.m(C)[ps].m(U)[ps].m | | p.m(A)p.m(A)p.m(U)[ps].m(A) | |
| | | (U) | | [ps].m(A) | |
| 6 | dual | Guide Strand 1 | 14 | [5am6_p_m](G)[ps].m(G)[ps].m | 89 |
| | | [5vp_una2moe](U)[ps].[fl2r](U)[ps].m | | (C)p.m(A)p.m(A)p.m(A)p.[fl2r](G) | |
| | | (A)p.m(U)p.m(U)p.[fl2r](A)p.m(U) | | p.[fl2r](A)p.[fl2r](A)p.m(C)p.m | |
| | | p.m(U)p.m(U)p.m(G)p.m(U)p.m(U) | | (A)p.m(A)p.m(A)p.m(U)p.m(A) | |
| | | p.m(C)p.[fl2r](U)p.m(U)p.[f12r](U) | | p.m(A)p.m(U)p.m(A)p.m(A)[ps].d | |
| | | p.m(G)p.m(C)p.m(C)[ps].m(U)[ps].m | | (T)[ps].d(T)[ps].d(T)[ps].m | |
| | | (U) | | | |
| | siMSTN | Guide Strand 2 | 14 | (G)p.m(G)p.m(C)p.m(A)p.m(A) | |
| | | [5vp_una2moe](U)[ps].[fl2r](U)[ps].m | | p.m(A)p.[fl2r](G)p.[fl2r](A)p.[fl2r] | |
| | | (A)p.m(U)p.m(U)p.[fl2r](A)p.m(U)p.m | | (A)p.m(C)p.m(A)p.m(A)p.m(A) | |
| | | (U)p.m(U)p.m(G)p.m(U)p.m(U) | | p.m(U)p.m(A)p.m(A)p.m(U)[ps].m | |
| | | p.m(C)p.[fl2r](U)p.m(U)p.[fl2r](U) | | (A)[ps].m(A) | |
| | | p.m(G)p.m(C)p.m(C)[ps].m(U)[ps].m | | | |
| | | (U) | | | |
| 7 | dual | Guide Strand 1 | 14 | [5am6_p_m](G)[ps].m(G)[ps].[rc22] | 90 |
| | pos3.C22. | [5vp_una2moe](U)[ps].[fl2r](U)[ps].m | | (C)p.m(A)p.m(A)p.m(A)p.[fl2r] | |
| | | (A)p.m(U)p.m(U)p.[fl2r](A)p.m(U) | | (G)p.[fl2r](A)p.[fl2r](A)p.m(C) | |
| | | p.m(U)p.m(U)p.m(G)p.m(U)p.m(U) | | p.m(A)p.m(A)p.m(A)p.m(U)p.m | |
| | | p.m(C)p.[fl2r](U)p.m(U)p.[fl2r](U) | | (A)p.m(A)p.m(U)p.m(A)p.m(A) | |
| | | p.m(G)p.m(C)p.m(C)[ps].m(U)[ps].m | | [ps].d(T)[ps].d(T)[ps].d(T)[ps].m | |
| | | (U) | | (G)p.m(G)p.m(C)p.m(A)p.m(A) | |
| | SIMSTN | Guide Strand 2 | 14 | p.m(A)p.[fl2r](G)p.[fl2r](A)p.[fl2r] | |
| | | [5vp_una2moe](U)[ps].[fl2r](U)[ps].m | | (A)p.m(C)p.m(A)p.m(A)p.m | |
| | | (A)p.m(U)p.m(U)p.[fl2r](A)p.m(U) | | (A)p.m(U)p.m(A)p.m(A)p.m(U) | |
| | | p.m(U)p.m(U)p.m(G)p.m(U)p.m(U) | | [ps].m(A)[ps].m(A) | |
| | | p.m(C)p.[f12r](U)p.m(U)p.[f12r](U) | | | |
| | | p.m(G)p.m(C)p.m(C)[ps].m(U)[ps].m | | | |
| | | (U) | | | |
| 8 | pos6.C16. | [5vp_una2moe](U)[ps].[fl2r](U)[ps].m | 14 | [5am6_p_m](G)[ps].m(G)[ps].m | 91 |
| | SIMSTN | (A)p.m(U)p.m(U)p.[fl2r](A)p.m(U) | | (C)p.m(A)p.m(A)p.[rc16](A)p.[f12r] | |
| | DAR1 | p.m(U)p.m(U)p.m(G)p.m(U)p.m(U) | | (G)p.[fl2r](A)p.[fl2r](A)p.m(C) | |
| | | p.m(C)p.[fl2r](U)p.m(U)p.[fl2r](U) | | p.m(A)p.m(A)p.m(A)p.m(U)p.m | |
| | | p.m(G)p.m(C)p.m(C)[ps].m(U)[ps].m(U) | | (A)p.m(A)p.m(U)p.m(A)p.m(A) | |
| | | | | [ps].d(T)[ps].d(T)[ps].d(T)[ps]. | |
| | | | | m(G)p.m(G)p.m(C)p.m(A)p.m(A) | |
| | | | | p.m(A)p.[fl2r](G)p.[fl2r](A)p.[fl2r] | |
| | | | | (A)p.m(C)p.m(A)p.m(A)p.m | |
| | | | | (A)p.m(U)p.m(A)p.m(A)p.m(U) | |
| | | | | [ps].m(A)[ps].m(A) | |
| 9 | dual | Guide Strand 1 | 14 | [5am6_p_m](G)[ps].m(G)[ps].m | 92 |
| | pos6.C16x | [5vp_una2moe](U)[ps].[fl2r](U)[ps].m | | (C)p.m(A)p.m(A)p.[rc16](A)p.[fl2r] | |
| | 2.siMSTN | (A)p.m(U)p.m(U)p.[fl2r](A)p.m(U) | | (G)p.[fl2r](A)p.[fl2r](A)p.m(C) | |
| | | p.m(U)p.m(U)p.m(G)p.m(U)p.m(U) | | p.m(A)p.m(A)p.m(A)p.m(U)p.m | |
| | | p.m(C)p.[fl2r](U)p.m(U)p.[fl2r](U) | | (A)p.m(A)p.m(U)p.m(A)p.m(A) | |
| | | p.m(G)p.m(C)p.m(C)[ps].m(U)[ps].m | | [ps].d(T)[ps].d(T)[ps].d(T)[ps].m | |
| | | (U) | | (G)p.m(G)p.m(C)p.m(A)p.m(A) | |
| | | Guide Strand 2 | 14 | p.[rc16](A)p.[fl2r](G)p.[fl2r](A) | |
| | | [5vp_una2moe](U)[ps].[fl2r](U)[ps]..m | | p.[fl2r](A)p.m(C)p.m(A)p.m(A) | |
| | | (A)p.m(U)p.m(U)p.[fl2r](A)p.m(U) | | p.m(A)p.m(U)p.m(A)p.m(A)p.m | |
| | | p.m(U)p.m(U)p.m(G)p.m(U)p.m(U) | | (U)[ps].m(A)[ps].m(A) | |
| | | p.m(C)p.[fl2](U)p.m(U)p.[fl2r](U) | | | |
| | | p.m(G)p.m(C)p.m(C)[ps].m(U)[ps].m | | | |
| | | (U) | | | |

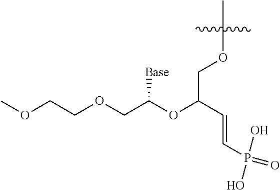

5vp_una2moe 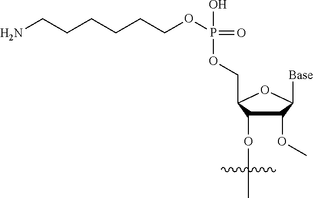
5am6_p_m 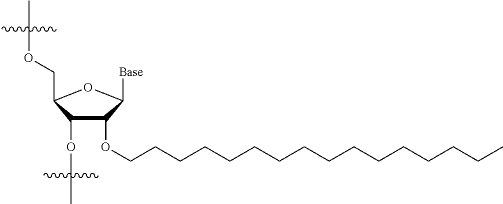
RC16 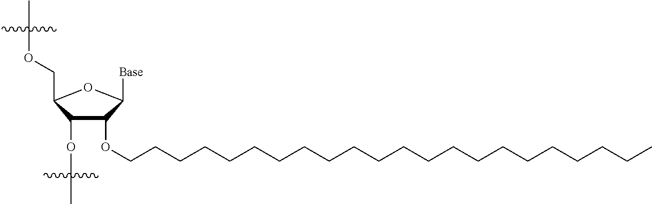
Rc

In Table 70, m indicates the 2′-O-methyl (2′-OMe) ribose modification; d indicates the 2′-deoxy ribose modification; [fl2r] indicates the 2′-deoxy-2′-fluoro(2′—F) ribose modification; [ps] indicates phosphorothioate (PS) linkage; p indicates phosphodiester (PO) linkage.

### Results

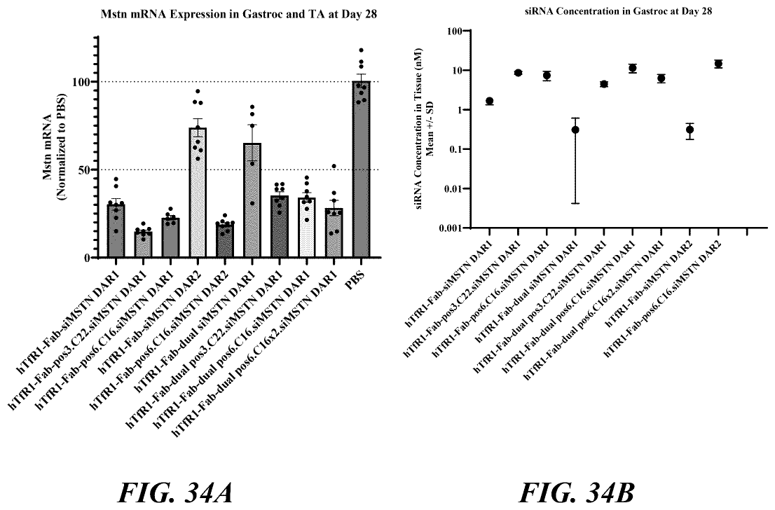

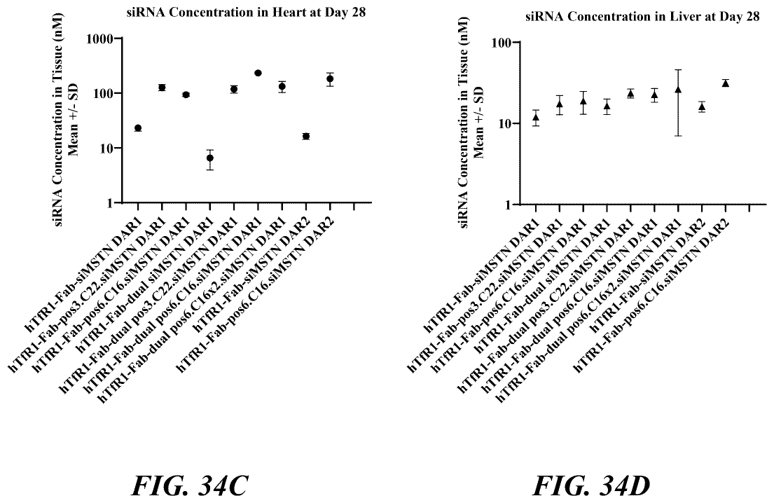

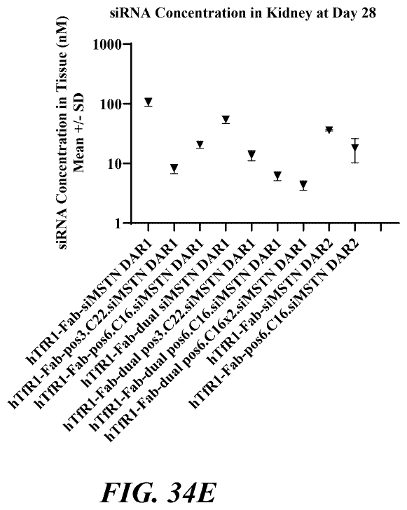

As shown in FIGS. 34A-34E, MSTN siRNA tissue concentrations and MSTN mRNA expression levels were assessed with hTfR1-Fab-siMSTN DAR1, hTfR1-Fab-pos3.C22.siMSTN DAR1, hTfR1-Fab-pos6.C16.siMSTN DAR1, hTfR1-Fab-pos6.C16.siMSTN DAR2, hTfR1-Fab-dual siMSTN DAR1, hTfR1-Fab-dual pos3.C22.siMSTN DAR1, hTfR1-Fab-dual pos6.C16.siMSTN DAR1, hTfR1-Fab-dual pos6.C16×2.siMSTN DAR1, and hTfR1-Fab-siMSTN DAR2 in the gastrocnemius (gastroc), tibialis anterior (TA), heart, kidney, and liver obtained from mice following a single intravenous (IV) injection at Day 0.

At day 28, siRNA tissue concentrations were quantified in tissue obtained from humanized mice that were administered a single intravenous injection of hTfR1-Fab-siMSTN DAR1, hTfR1-Fab-pos3.C22.siMSTN DAR1, hTfR1-Fab-pos6.C16.siMSTN DAR1, hTfR1-Fab-pos6.C16.siMSTN DAR2, hTfR1-Fab-dual siMSTN DAR1, hTfR1-Fab-dual pos3.C22.siMSTN DAR1, hTfR1-Fab-dual pos6.C16.siMSTN DAR1, hTfR1-Fab-dual pos6.C16×2.siMSTN DAR1, and hTfR1-Fab-siMSTN DAR2 FabOCs. The FabOCs containing a lipid on the passenger strand were able to deliver higher amounts of siRNA to all muscle tissues than the non-lipid Fab-OCs (FIG. 34B-34E). Lipid FabOCs delivered higher levels of siRNA in cardiac tissue than the levels in skeletal muscles.

At day 28, mRNA expression levels were quantified in tissue obtained from humanized mice that were administered a administered a single intravenous of hTfR1-Fab-siMSTN DAR1, hTfR1-Fab-pos3.C22.siMSTN DAR1, hTfR1-Fab-pos6.C16.siMSTN DAR1, hTfR1-Fab-pos6.C16.siMSTN DAR2, hTfR1-Fab-dual siMSTN DAR1, hTfR1-Fab-dual pos3.C22.siMSTN DAR1, hTfR1-Fab-dual pos6.C16.siMSTN DAR1, hTfR1-Fab-dual pos6.C16×2.siMSTN DAR1, and hTfR1-Fab-siMSTN DAR2 FabOCs. Lipid FabOCs administered intravenously were able to decrease mRNA expression levels in skeletal muscle tissue and cardiac tissue (FIG. 34A) to a greater degree than the DAR1 non-lipid Fab-OCs. The FabOCs containing a lipid on the passenger strand were able to decrease mRNA expression levels in skeletal muscle tissue and cardiac tissue (FIG. 34A) to a greater degree than non-lipid Fab-OCs.

Overall, lipid-FabOCs with a single intravenous injection of dual MSTN hTfR1-Fab-dual siMSTN DAR1, hTfR1-Fab-dual pos3.C22.siMSTN DAR1, hTfR1-Fab-dual pos6.C16.siMSTN DAR1, hTfR1-Fab-dual pos6.C16×2.siMSTN DAR1 FabOCs were able to deliver higher amount of siRNA to tissues and decrease mRNA expression levels in skeletal muscle and cardiac tissues to a greater extent than non-lipid dual MSTN hTfR1-Fab-dual siMSTN DAR1.

### Example 24: PKPD of Lipid Fab siRNA Conjugates (FabOCs) Following IV Infusion or Subcutaneous Injection in Cynomolgus Monkeys

A 21-mer double-stranded siRNA with 19 bases of complementarity and 3′ dinucleotide overhangs was designed against human DMPK. The sequence (5′ to 3′) of the guide/antisense strand was UUCGAAGACAGUUCUAGGGUU (SEQ ID NO: 7). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker. The passenger strand of the lipid Fab-OCs was modified with a 2′-O—C22 at position 3 nucleotide.

### Conjugate Synthesis and Characterization

The CD71 Fab-siRNA conjugates were made, purified and characterized as described in Examples 1-3. All conjugates were made through cysteine conjugation with a SMCC linker. Conjugates were characterized chromatographically as described in Table 71.

TABLE 71
HPLC Retention Time (RT) in Minutes

| | | RT, SAX | RT, SEC |
|---|---|---|---|
| Group(s) | Conjugate | Method-3 | Method-2 |
| 1 | hTfR1-Fab-siDMPK DAR1 | 3.359 | 15.942 |
| 2 | hTfR1-Fab-pos3.C22.siDMPK DAR1 | 3.521 | 16.165 |
| 3 | hTfR1-Fab-pos3.C22.siDMPK DAR2 | 4.029 | 14.890 |

### In Vivo Study Design

The conjugates were assessed for their ability to mediate mRNA downregulation of myotonic dystrophy protein kinase (DMPK) in skeletal muscle in vivo in cynomolgus monkeys. Monkeys were dosed via intravenous (IV) infusion or subcutaneous (SC) administration with PBS vehicle control or the indicated doses as shown in Table 72. Punch biopsies were collected from gastrocnemius and vastus lateralis pre-dose and Day 8 and Day 29. After 57 days, gastrocnemius (gastroc), vastus lateralis, bicep, liver, kidney, and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using the appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the pre-dose control by taking a second difference (ΔΔCt).

TABLE 72
In vivo study design testing PKPD of DMPK siRNA Fab AOCs in Cynomolgus
Monkeys

| | | siRNA | | | | | |
|---|---|---|---|---|---|---|---|
| | | dose | | | | Muscle Biopsy | |
| | | (mg/ | Doses | Dose | | Samples | Harvest |
| Group | Test Article | kg) | (#) | Schedule | ROA | (Days) | (Day) |
| 1 | hTfR1-mAb- | 1 | 1 | single | IV | Pre-dose, 8, 29 | 57 |
| | siDMPK DAR1 | | | | | | |
| 2 | hTfR1-Fab- | 1 | 1 | single | IV | Pre-dose, 8, 29 | 57 |
| | pos3.C22.siDMPK | | | | | | |
| | DAR1 | | | | | | |
| 3 | hTfR1-Fab- | 2 | 1 | single | IV | Pre-dose, 8, 29 | 57 |
| | pos3.C22.siDMPK | | | | | | |
| | DAR2 | | | | | | |
| 4 | hTfR1-Fab- | 3 | 1 | single | SC | Pre-dose, 8, 29 | 57 |
| | pos3.C22.siDMPK | | | | | | |
| | DAR1 | | | | | | |

TABLE 73
siRNA Sequences used in Example 24

| | | | SEQ | | SEQ |
|---|---|---|---|---|---|
| Compound | siRNA | | ID | | ID |
| No. | Ref. | siRNA Guide Sequence | NO: | siRNA Passenger Sequence | NO: |
| 1 | siDMPK | [5vp_una2moe](U)[ps].[fl2r](U) | 17 | [5vp_una2moe](U)[ps].[fl2r](U) | 17 |
| | | [ps].m(C)p.m(G)p.m(A)p.[fl2r](A) | | [ps].m(C)p.m(G)p.m(A)p.[fl2r](A) | |
| | | p.m(G)p.m(A)p.m(C)p.m(A)p.m | | p.m(G)p.m(A)p.m(C)p.m(A)p.m | |
| | | (G)p.m(U)p.m(U)p.[fl2r](C)p.m | | (G)p.m(U)p.m(U)p.[fl2r](C)p.m | |
| | | (U)p.[fl2r](A)p.m(G)p.m(G)p.m | | (U)p.[fl2r](A)p.m(G)p.m(G)p.m | |
| | | (G)[ps].m(U)[ps].m(U) | | (G)[ps].m(U)[ps].m(U) | |
| 7 | Pos3.C22.s | [5vp_una2moe](U)[ps].[fl2r](U) | 17 | {[5am6_p_m](C)[ps].m(C)[ps].[rc22] | 93 |
| | IDMPK | [ps].m(C)p.m(G)p.m(A)p.[fl2r](A) | | (C)p.m(U)p.m(A)p.m(G)p.[fl2r] | |
| | | p.m(G)p.m(A)p.m(C)p.m(A)p.m | | (A)p.[fl2r](A)p.[fl2r](C)p.m | |
| | | (G)p.m(U)p.m(U)p.[fl2r](C)p.m | | (U)p.m(G)p.m(U)p.m(C)p.m(U) | |
| | | (U)p.[fl2r](A)p.m(G)p.m(G)p.m | | p.m(U)p.m(C)p.m(G)[ps].m(A)[ps].m | |
| | | (G)[ps].m(U)[ps].m(U) | | (A) | |

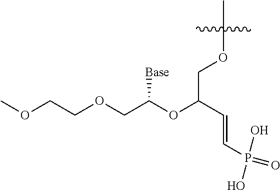

5vp_una2moe 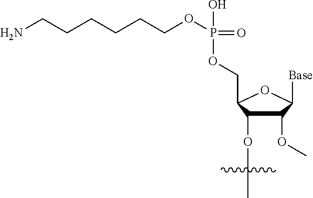
5am6_p_m 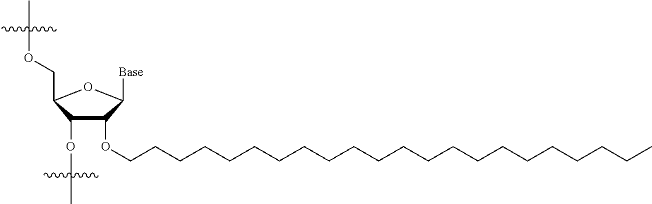
rc22

In Table 73, m indicates the 2′-O-methyl (2′-OMe) ribose modification; [fl2r] indicates the 2′-deoxy-2′-fluoro(2′—F) ribose modification; [ps] indicates phosphorothioate (PS) linkage; p indicates phosphodiester (PO) linkage.

### Results

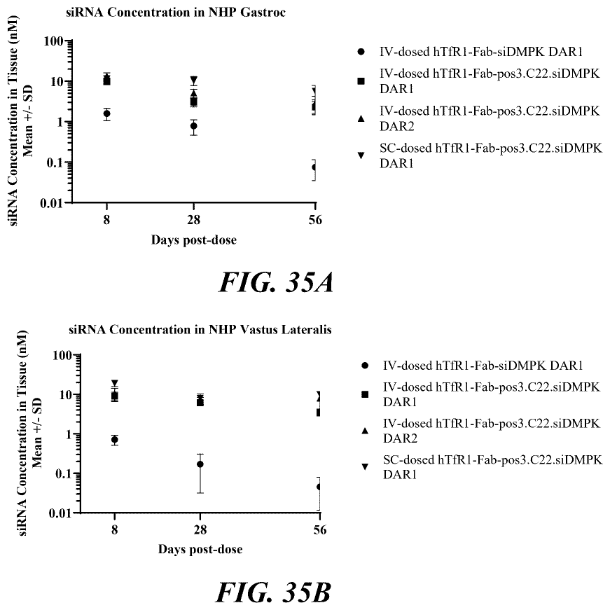

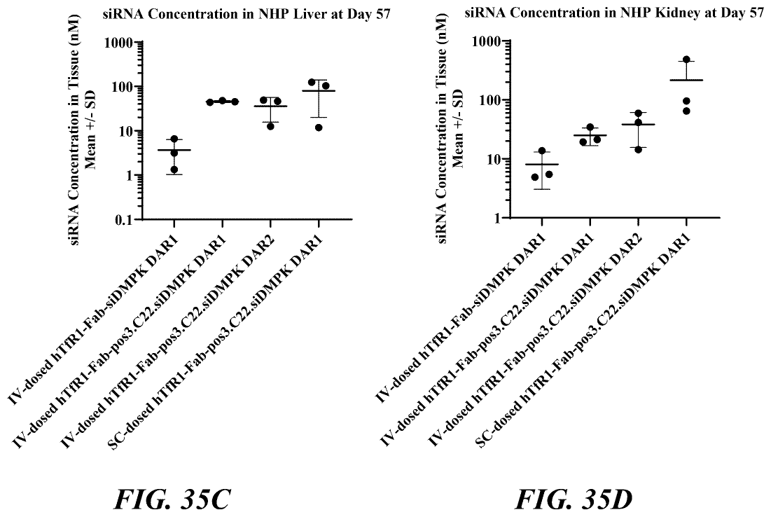

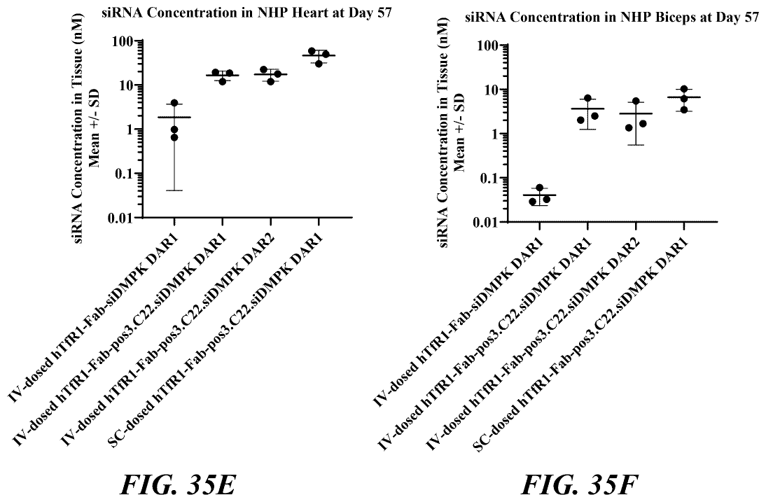

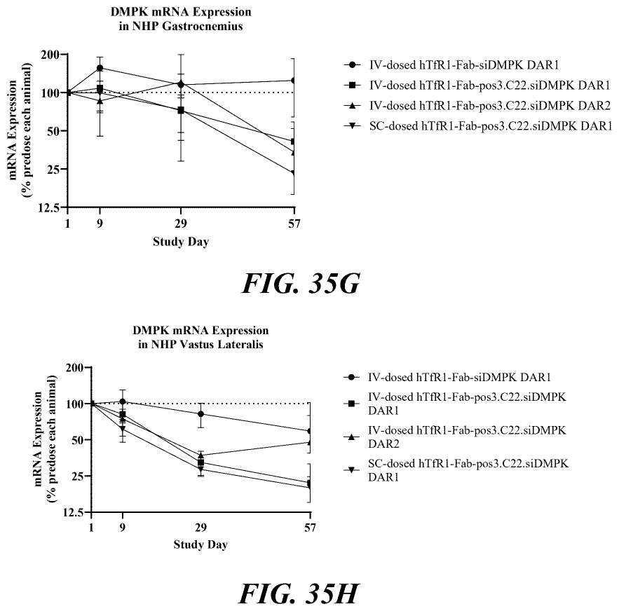

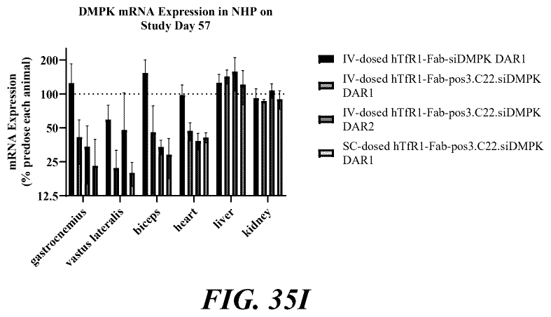

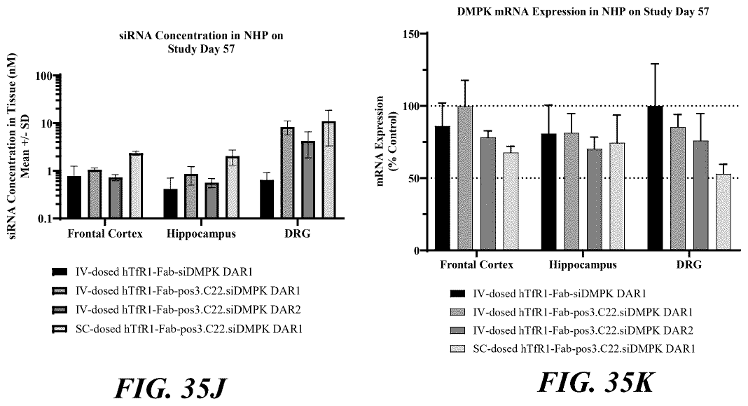

As shown in FIGS. 35A-35K, DMPK siRNA tissue concentrations and DMPK mRNA expression levels were assessed with DAR1 lipid FabOCs after intravenous or subcutaneous administration with the C22 hydrocarbon chain conjugated at position 3 of the passenger strand, intravenous administration of DAR2 lipid FabOCs after intravenous or subcutaneous administration with the C22 hydrocarbon chain conjugated at position 3 of the passenger strand, or with intravenous administration of hTfR1-Fab-siDMPK DAR1 in the gastrocnemius (gastroc), vastus lateralis, bicep, liver, kidney, and heart muscle, frontal cortex, hippocampus, and dorsal root ganglion
tissues obtained from cynomolgus monkeys following a single intravenous (IV) or subcutaneous injection at Day 0.

At day 57, siRNA tissue concentrations were quantified in tissue obtained from cynomolgus monkeys that were administered intravenous or subcutaneous administration with the C22 hydrocarbon chain conjugated at position 3 of the passenger strand, intravenous administration of DAR2 lipid FabOCs after intravenous or subcutaneous administration with the C22 hydrocarbon chain conjugated at position 3 of the passenger strand, or with intravenous administration of hTfR1-Fab-siDMPK DAR1. The lipid-FabOCs were able to deliver higher amount of siRNA to all muscle tissues than the non-lipid Fab AOCs (FIGS. 35A-35F, 35J). Lipid FabOCs delivered higher levels of siRNA in cardiac tissue than the levels in skeletal muscles in addition to central and peripheral nervous system tissues like frontal cortex, hippocampus, and dorsal root ganglion.

At days 8, 28, and 57, mRNA expression levels were quantified in tissue obtained from cynomolgus monkeys that were administered intravenous or subcutaneous administration with the C22 hydrocarbon chain conjugated at position 3 of the passenger strand, intravenous administration of DAR2 lipid FabOCs after intravenous or subcutaneous administration with the C22 hydrocarbon chain conjugated at position 3 of the passenger strand, or with intravenous administration of hTfR1-Fab-siDMPK DAR1. Lipid FabOCs administered with intravenous or subcutaneous dosing were able to decrease mRNA expression levels in skeletal and cardiac muscle in additional to central and peripheral nervous system tissues like frontal cortex, hippocampus, and dorsal root ganglion (FIGS. 35G-35I, 35K) to a greater degree than the DAR1 non-lipid mAb and Fab AOCs.

Overall, DAR1 lipid-FabOC administered intravenous or subcutaneous administration with the C22 hydrocarbon chain conjugated at position 3 of the passenger strand, intravenous administration of DAR2 lipid FabOCs after intravenous or subcutaneous administration with the C22 hydrocarbon chain conjugated at position 3 of the passenger strand were able to deliver higher amount of siRNA to tissues and decrease mRNA expression levels in skeletal and cardiac muscle in additional to central and peripheral nervous system tissues like frontal cortex, hippocampus, and dorsal root ganglion, to a greater extent than DAR1 non-lipid hTfR1-mAb-siDMPK administered intravenously.

### Example 25: Evaluation of TfR1-Targeted siRNA AOCs Containing Cleavable Lipids in Wild Type Mice

DMPK: A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker.

### Conjugate Synthesis and Characterization

The CD71 mAb-siRNA conjugates were made, purified and characterized as described in Example 3. All conjugates were made through cysteine conjugation with a SMCC linker. Conjugates were characterized chromatographically as described in Table 74.

TABLE 74
HPLC Retention Time (RT) in Minutes

| | | RT, SAX | RT, SEC |
|---|---|---|---|
| Group(s) | Conjugate | Method-5 | Method-2 |
| 1 | mTfR1-mAb-siDMPK DAR1 | 3.134 | 3.063 |
| 2 | mTfR1-mAb-pos.3.C22.siDMPK DAR1 | 3.181 | 3.109 |
| 3 | mTfR1-mAb-pos3.tzC22.siDMPK DAR1 | 3.427 | 3.228 |
| 4 | mTfR1-mAb-pos3.tz(me)amideC22.siDMPK DAR1 | 3.474 | 3.239 |
| 5 | mTfR1-mAb-pos3.tz(EG)amideC22.siDMPK DAR1 | 3.393 | 3.216 |
| 6 | mTfR1-mAb-pos3.tz(EG)esterC22.siDMPK DAR1 | 3.387 | 3.216 |
| 7 | mTfR1-mAb- | 3.466 | 3.198 |
| | pos3.tz(cleavable)Gluc.C22.siDMPK DAR1 | | |
| 8 | mTfR1-mAb-pos3.tz(non- | 3.482 | 3.240 |
| | cleavable)Gluc.C22.siDMPK DAR1 | | |
| 9 | mTfR1-mAb-pos3.tz(VC)C22.siDMPK DAR1 | 3.503 | 3.267 |
| 10 | mTfR1-mAb-pos3.tz(disulfide)C22.siDMPK DAR1 | 3.250 | 3.133 |

### In Vivo Study Design

The conjugates were assessed for their ability to mediate mRNA downregulation of myotonic dystrophy protein kinase (DDPK) in skeletal muscle in vivo in wildtype mice. Mice were dosed via intravenous (IV) injection with PBS vehicle control or the indicated AOCs at 10 mg/kg mAb protein dose, or 1 mg/kg for the DARM conjugates and 2 mg/kg for the DAR2 and 4 mg/kg for the DAR4 conjugates as shown in Table 75. After 1, 3, and 5 months, gastrocnemius (gastroc) and heart muscle tissues were harvested and snap-frozen in liquid nitrogen. mRNA knockdown in target tissue was determined using a comparative qPCR assay. Total RNA was extracted from the tissue, reverse transcribed and mRNA expression levels were quantified using TaqMan qPCR, using appropriately designed primers and probes. PPIB (housekeeping gene) was used as an internal RNA loading control, results were calculated by the comparative Ct method, where the difference between the target gene Ct value and the PPIB Ct value (ΔCt) is calculated and then further normalized relative to the PBS control group by taking a second difference (ΔΔCt).

TABLE 75
In Vivo Study Design Testing TfR1-Targeted siRNA AOCs Containing
Cleavable Lipids in Wild Type Mice

| | | | | | siRNA | | |
|---|---|---|---|---|---|---|---|
| | | siRNA | | | Dose | Doses | Harvest |
| Group | Test Article | Target | N | DAR | (mg/kg) | (#) | (month) |
| 1 | mTfR1-mAb-siDMPK DAR1 | DMPK | 4 | 1 | 0.5 | 1 | 1, 3 |
| 2 | mTfR1-mAb-pos.3.C22.siDMPK DAR1 | DMPK | 4 | 1 | 0.5 | 1 | 1, 3 |
| 3 | mTfR1-mAb-pos3.tzC22.siDMPK DAR1 | DMPK | 4 | 1 | 0.5 | 1 | 1, 3 |
| 4 | mTfR1-mAb- | DMPK | 4 | 1 | 0.5 | 1 | 1, 3 |
| | pos3.tz(me)amideC22.siDMPK DAR1 | | | | | | |
| 5 | mTfR1-mAb- | DMPK | 4 | 1 | 0.5 | 1 | 1, 3 |
| | pos3.tz(EG)amideC22.siDMPK DAR1 | | | | | | |
| 6 | mTfR1-mAb- | DMPK | 4 | 1 | 0.5 | 1 | 1, 3 |
| | pos3.tz(EG)esterC22.siDMPK DAR1 | | | | | | |
| 7 | mTfR1-mAb- | DMPK | 4 | 1 | 0.5 | 1 | 1, 3 |
| | pos3.tz(cleavable)Gluc.C22.siDMPK | | | | | | |
| | DAR1 | | | | | | |
| 8 | mTfR1-mAb-pos3.tz(non- | DMPK | 4 | 1 | 0.5 | 1 | 1, 3 |
| | cleavable)Gluc.C22.siDMPK DAR1 | | | | | | |
| 9 | mTfR1-mAb- | DMPK | 4 | 1 | 0.5 | 1 | 1, 3 |
| | pos3.tz(VC)C22.siDMPK DAR1 | | | | | | |
| 10 | PBS | DMPK | 4 | N/A | N/A | 1 | 1, 3 |

* All animals used in the experiment in Table 75 have body weights of 0.035 kg, and were administered with a single dose with a dose volume of 5.0 ml/kg

TABLE 76

| | | | SEQ | | SEQ |
|---|---|---|---|---|---|
| Compound | | | ID | | ID |
| No. | siRNA Ref. | siRNA Guide Sequence | NO: | siRNA Passenger Sequence | NO: |
| 1 | (vp)siDMPK | [5vp_una2moe](U)[ps]. [fl2r](A) | 12 | [5am6_p_m](C)[ps].m(C)[ps].m | |
| | | [ps].m(A)p.m(U)p.m(A)p.[fl2r] | | (G)p.m(A)p.m(C)p.m(A)p.[fl2r] | |
| | | (C)p.m(C)p.m(G)p.m(A)p.m | | (U)p.[fl2r](U)p.[fl2r](C)p.m | |
| | | (G)p.m(G)p.m(A)p.m(A)p.[fl2r] | | (C)p.m(U)p.m(C)p.m(G)p.m(G) | |
| | | (U)p.m(G)p.[fl2r](U)p.m(C) | | p.m(U)p.m(A)p.m(U)[ps].m | |
| | | p.m(G)p.m(G)[ps].m(U)[ps].m | | (U)[ps].m(A) | |
| | | (U) | | | |
| 2 | Pos3.C22.siD | [5vp_una2moe](U)[ps].[fl2r](A) | 12 | [5am6_p_m](C)[ps].m(C)[ps].[rc22] | 81 |
| | MPK | [ps].m(A)p.m(U)p.m(A)p.[fl2r] | | (G)p.m(A)p.m(C)p.m(A) | |
| | | (C)p.m(C)p.m(G)p.m(A)p.m | | p.[fl2r](U)p.[fl2r](U)p.[fl2r](C) | |
| | | (G)p.m(G)p.m(A)p.m(A)p.[fl2r] | | p.m(C)p.m(U)p.m(C)p.m(G)p.m | |
| | | (U)p.m(G)p.[fl2r](U)p.m(C) | | (G)p.m(U)p.m(A)p.m(U)[ps].m | |
| | | p.m(G)p.m(G)[ps].m(U)[ps].m | | (U)[ps].m(A) | |
| | | (U) | | | |
| 3 | Pos3.tz.siDM | [5vp_una2moe](U)[ps].[fl2r](A) | 12 | [5am6_p_m](C)[ps].m(C)[ps].[rctz] | 94 |
| | PK | [ps].m(A)p.m(U)p.m(A)p.[fl2r] | | (G)p.m(A)p.m(C)p.m(A)p.[fl2r] | |
| | | (C)p.m(C)p.m(G)p.m(A)p.m | | (U)p.[fl2r](U)p.[fl2r](C)p.m | |
| | | (G)p.m(G)p.m(A)p.m(A)p.[fl2r] | | (C)p.m(U)p.m(C)p.m(G)p.m | |
| | | (U)p.m(G)p.[fl2r](U)p.m(C) | | (G)p.m(U)p.m(A)p.m(U)[ps].m | |
| | | p.m(G)p.m(G)[ps].m(U)[ps].m | | (U)[ps].m(A) | |
| | | (U) | | | |

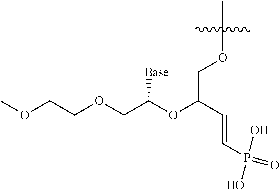

5vp_una2moe 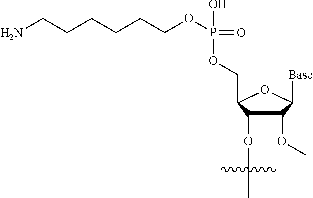
5am6_p_m 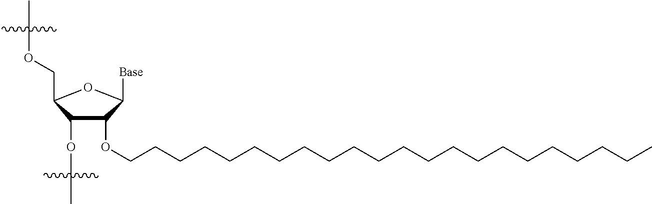
Rc22 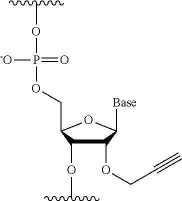
Rctz 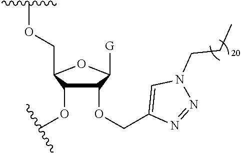
tzC22 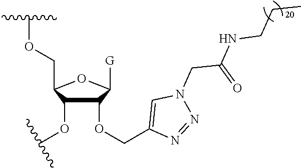
tz(Me)amideC22tz(EG)amideC22 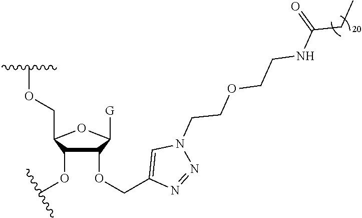
tz(EG)amideC22 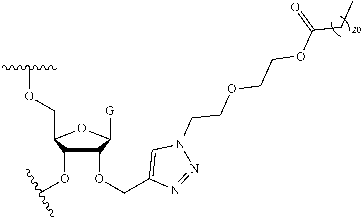
tz(EG)esterC22 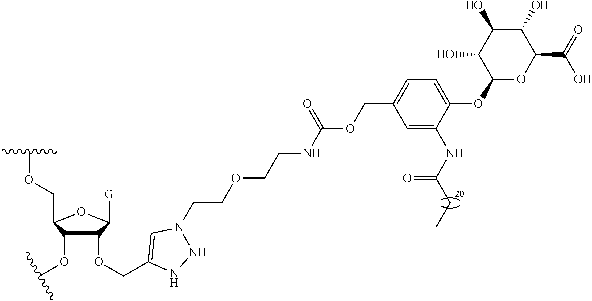
tz(cleavable)Gluc C22 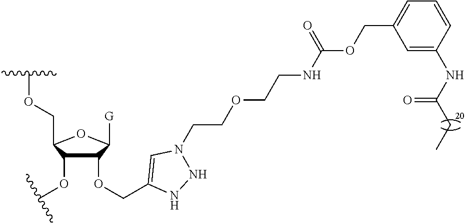
tz(non-cleavable)Gluc.C22 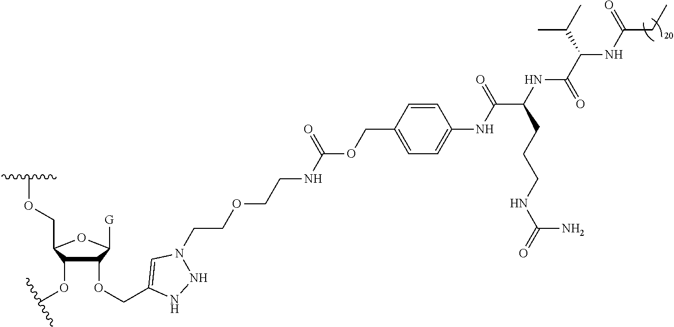
tz(VC)C22

In Table 76, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage

### Results

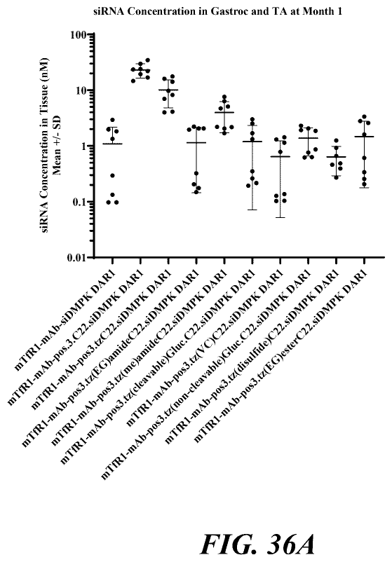

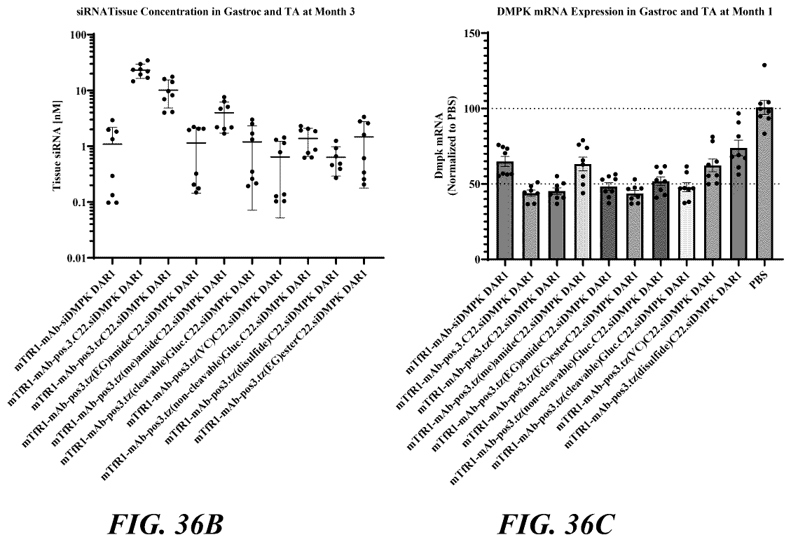

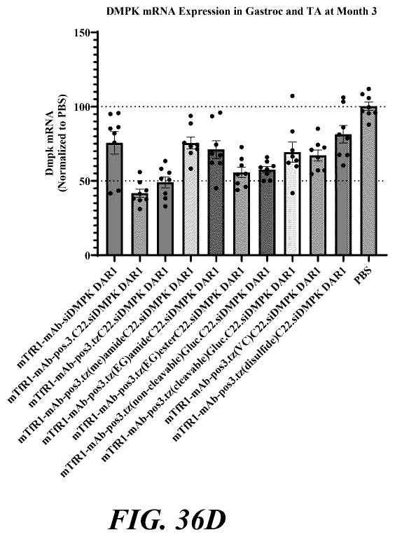

As shown in FIGS. 36A-36D, DMPK siRNA tissue concentrations and DMPK mRNA expression levels were assessed with DAR1 TfR1-targeting AOCs at Month 1 and Month 3 in the gastroc and TA obtained from mice following a single intravenous (IV) injection of the AOCs at Day 0.

At Month 1 and 3, DMPK siRNA tissue concentrations were quantified in tissue obtained from wild type mice that were administered a single intravenous injection of DAR1 TfR1-targeting AOCs modified at passenger position 3 according to Table 75 and compared to non-lipid DAR1 TfR1-targeting AOC. As shown in FIG. 36A-36B, the lipid AOCs with position 3 2′—O-ether lipids increased the muscle tissue exposure compared to the cleavable lipids and the non-lipid AOC control. As shown in FIG. 36C-36D, the lipid AOCs with position 3 2′—O-ether lipids were able to reduce mRNA expression levels more than non-lipid AOCs in all muscle tissues.

### Example 26: In Vitro Evaluation of siRNAs that have been Modified with C16 Lipid Along Guide and Passenger Strand

### Luciferase-Reporter Assay in HEK293 Cells

HEK 293 cells were engineered to express DMPK within the psiCHECK™-2 vector (Promega). Cells were grown in DMEM supplemented with 10% FBS (Corning). Cells were seeded in triplicate at 20,000 cells/well in 96-well tissue culture plates without antibiotics 24 hrs prior to transfection. On the day of transfection, siRNAs were formulated with RNAiMax (Life Technologies, Waltham, MA, USA) and OptiMEM (Life Technologies, Waltham, MA, USA), transfection was performed following manufacturer's instructions. 48 h after transfection, firefly and Renilla luciferase activities were measured using the Dual-Glo® Luciferase Assay System (Promega, Madison, WI, USA) according to manufacturer's instructions. Luminescence signal was read on Infinite® M200 Pro plate reader (Tecan, Mannedorf, Switzerland). The relative Renilla/firefly luciferase ratio was calculated by first subtracting the background from the luciferase activity measurements (no-reporter transfection controls used as background reference) and then generating the relative ratio to no-siRNA transfection control.

TABLE 77
siRNA Sequences used in Example 26

| | | | SEQ | | SEQ |
|---|---|---|---|---|---|
| Compound | | | ID | | ID |
| No. | siRNA Ref. | siRNA Guide Sequence | NO: | siRNA Passenger Sequence | NO: |
| 1 | vp.siDMPK | [5vp_una2moe](U)[ps].[f12r](U) | 17 | {[5am6_p_m](C)[ps].m(C)[ps].m | 95 |
| | | [ps].m(C)p.m(G)p.m(A)p.[fl2r](A) | | (C)p.m(U)p.m(A)p.m(G)p.[fl2r] | |
| | | p.m(G)p.m(A)p.m(C)p.m(A)p.m | | (A)p.[f12r](A)p.[fl2r](C)p.m(U) | |
| | | (G)p.m(U)p.m(U)p.[fl2r](C)p.m | | p.m(G)p.m(U)p.m(C)p.m(U)p.m | |
| | | (U)p.[fl2r](A)p.m(G)p.m(G)p.m | | (U)p.m(C)p.m(G)[ps].m(A)[psJ.m | |
| | | (G)[ps].m(U)[ps].m(U) | | (A) | |
| 2 | Pos3.C22.si | [5vp_una2moe](U)[ps].[fl2r](U) | 17 | {[5am6_p_m](C)[ps].m(C)[ps].[rc22] | 93 |
| | DMPK | [ps].m(C)p.m(G)p.m(A)p.[fl2r](A) | | (C)p.m(U)p.m(A)p.m(G)p.[f12r] | |
| | | p.m(G)p.m(A)p.m(C)p.m(A)p.m | | (A)p.[fl2r](A)p.[fl2r](C)p.m | |
| | | (G)p.m(U)p.m(U)p.[fl2r](C)p.m | | (U)p.m(G)p.m(U)p.m(C)p.m(U) | |
| | | (U)p.[fl2r](A)p.m(G)p.m(G)p.m | | p.m(U)p.m(C)p.m(G)[ps].m(A)[ps].m | |
| | | (G)[ps].m(U)[ps].m(U) | | (A) | |
| 3 | Pos1.C16.PS | [5vp_una2moe](U)[ps].[fl2r](U) | 17 | {[rc16](C)[ps].m(C)[ps].m(C)p. | 96 |
| | .siDMPK | [ps].m(C)p.m(G)p.m(A)p.[fl2r](A) | | m(U)p.m(A)p.m(G)p.[fl2r](A)p.[fl2r] | |
| | | p.m(G)p.m(A)p.m(C)p.m(A)p.m | | (A)p.[fl2r](C)p.m(U)p.m(G) | |
| | | (G)p.m(U)p.m(U)p.[fl2r](C)p.m | | p.m(U)p.m(C)p.m(U)p.m(U)p.m | |
| | | (U)p.[fl2r](A)p.m(G)p.m(G)p.m | | (C)p.m(G)[ps].m(A)[ps].m(A) | |
| | | (G)[ps].m(U)[ps].m(U) | | | |
| 4 | Pos2.C16.PS | [5vp_una2moe](U)[ps].[fl2r](U) | 17 | {m(C)[ps].[rc16](C)[ps].m(C)p. | 97 |
| | .siDMPK | [ps].m(C)p.m(G)p.m(A)p.[fl2r](A) | | m(U)p.m(A)p.m(G)p.[fl2r](A)p.[fl2r] | |
| | | p.m(G)p.m(A)p.m(C)p.m(A)p.m | | (A)p.[fl2r](C)p.m(U)p.m(G) | |
| | | (G)p.m(U)p.m(U)p.[fl2r](C)p.m | | p.m(U)p.m(C)p.m(U)p.m(U)p.m | |
| | | (U)p.[fl2r](A)p.m(G)p.m(G)p.m | | (C)p.m(G)[ps].m(A)[ps].m(A)} | |
| | | (G)[ps].m(U)[ps].m(U) | | | |
| 5 | Pos3.C16.PS | [5vp_una2moe](U)[ps].[fl2r](U) | 17 | {m(C)[ps].m(C)[ps].[rc16](C)p.m | 98 |
| | .siDMPK | [ps].m(C)p.m(G)p.m(A)p.[fl2r](A) | | (U)p.m(A)p.m(G)p.[fl2r](A)p.[fl2r] | |
| | | p.m(G)p.m(A)p.m(C)p.m(A)p.m | | (A)p.[fl2r](C)p.m(U)p.m(G) | |
| | | (G)p.m(U)p.m(U)p.[fl2r](C)p.m | | p.m(U)p.m(C)p.m(U)p.m(U)p.m | |
| | | (U)p.[fl2r](A)p.m(G)p.m(G)p.m | | (C)p.m(G)[ps].m(A)[ps].m(A) | |
| | | (G)[ps].m(U)[ps].m(U) | | | |
| 6 | Pos4.C16.PS | [5vp_una2moe](U)[ps].[fl2r](U) | 17 | {m(C)[ps].m(C)[ps].m(C)p.[rc16] | 99 |
| | .siDMPK | [ps].m(C)p.m(G)p.m(A)p.[fl2r](A) | | (U)p.m(A)p.m(G)p.[fl2r](A)p.[f12r] | |
| | | p.m(G)p.m(A)p.m(C)p.m(A)p.m | | (A)p.[fl2r](C)p.m(U)p.m(G) | |
| | | (G)p.m(U)p.m(U)p.[fl2r](C)p.m | | p.m(U)p.m(C)p.m(U)p.m(U)p.m | |
| | | (U)p.[fl2r](A)p.m(G)p.m(G)p.m | | (C)p.m(G)[ps].m(A)[ps].m(A)} | |
| | | (G)[ps].m(U)[ps].m(U) | | | |
| 7 | Pos5.C16.PS | [5vp_una2moe](U)[ps].[fl2r](U) | 17 | {m(C)[ps].m(C)[ps].m(C)p.m(U) | 100 |
| | .siDMPK | [ps].m(C)p.m(G)p.m(A)p.[fl2r](A) | | p.[rc16](A)p.m(G)p.[f12r](A)p.[f12r] | |
| | | p.m(G)p.m(A)p.m(C)p.m(A)p.m | | (A)p.[fl2r](C)p.m(U)p.m(G) | |
| | | (G)p.m(U)p.m(U)p.[fl2r](C)p.m | | p.m(U)p.m(C)p.m(U)p.m(U)p.m | |
| | | (U)p.[fl2r](A)p.m(G)p.m(G)p.m | | (C)p.m(G)[ps].m(A)[ps].m(A)} | |
| | | (G)[ps].m(U)[ps].m(U) | | | |
| 8 | Pos6.C16.PS | [5vp_una2moe](U)[ps].[fl2r](U) | 17 | {m(C)[ps].m(C)[ps].m(C)p.m(U) | 101 |
| | .siDMPK | [ps].m(C)p.m(G)p.m(A)p.[fl2r](A) | | p.m(A)p.[rc16](G)p.[f12r](A)p.[fl2r] | |
| | | p.m(G)p.m(A)p.m(C)p.m(A)p.m | | (A)p.[fl2r](C)p.m(U)p.m(G) | |
| | | (G)p.m(U)p.m(U)p.[fl2r](C)p.m | | p.m(U)p.m(C)p.m(U)p.m(U)p.m | |
| | | (U)p.[fl2r](A)p.m(G)p.m(G)p.m | | (C)p.m(G)[ps].m(A)[ps].m(A)} | |
| | | (G)[ps].m(U)[ps].m(U) | | | |
| 9 | Pos7.C16.PS | [5vp_una2moe](U)[ps].[fl2r](U) | 17 | {m(C)[ps].m(C)[ps].m(C)p.m(U) | 102 |
| | .siDMPK | [ps].m(C)p.m(G)p.m(A)p.[fl2r](A) | | p.m(A)p.m(G)p.[rc16](A)p.[f12r] | |
| | | p.m(G)p.m(A)p.m(C)p.m(A)p.m | | (A)p.[fl2r](C)p.m(U)p.m(G)p.m | |
| | | (G)p.m(U)p.m(U)p.[fl2r](C)p.m | | (U)p.m(C)p.m(U)p.m(U)p.m(C) | |
| | | (U)p.[fl2r](A)p.m(G)p.m(G)p.m | | p.m(G)[ps].m(A)[ps].m(A)} | |
| | | (G)[ps].m(U)[ps].m(U) | | | |
| 10 | Pos8.C16.PS | [5vp_una2moe](U)[ps].[fl2r](U) | 17 | {m(C)[ps].m(C)[ps].m(C)p.m(U) | 103 |
| | .siDMPK | [ps].m(C)p.m(G)p.m(A)p.[fl2r](A) | | p.m(A)p.m(G)p.[fl2r](A)p.[rc16] | |
| | | p.m(G)p.m(A)p.m(C)p.m(A)p.m | | (A)p.[fl2r](C)p.m(U)p.m(G)p.m | |
| | | (G)p.m(U)p.m(U)p.[fl2r](C)p.m | | (U)p.m(C)p.m(U)p.m(U)p.m(C) | |
| | | (U)p.[fl2r](A)p.m(G)p.m(G)p.m | | p.m(G)[ps].m(A)[ps].m(A)} | |
| | | (G)[ps].m(U)[ps].m(U) | | | |
| 11 | Pos9.C16.PS | [5vp_una2moe](U)[ps].[fl2r](U) | 17 | {m(C)[ps].m(C)[ps].m(C)p.m(U) | 104 |
| | siDMPK | [ps].m(C)p.m(G)p.m(A)p.[fl2r](A) | | p.m(A)p.m(G)p.[f12r](A)p.[fl2r] | |
| | | p.m(G)p.m(A)p.m(C)p.m(A)p.m | | (A)p.[rc16](C)p.m(U)p.m(G)p.m | |
| | | (G)p.m(U)p.m(U)p.[fl2r](C)p.m | | (U)p.m(C)p.m(U)p.m(U)p.m(C) | |
| | | (U)p.[fl2r](A)p.m(G)p.m(G)p.m | | p.m(G)[ps].m(A)[ps].m(A)} | |
| | | (G)[ps].m(U)[ps].m(U) | | | |
| 12 | Pos10.C16.P | [5vp_una2moe](U)[ps].[fl2r](U) | 17 | {m(C)[ps].m(C)[ps].m(C)p.m(U) | 105 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.[rc16](U)p.m(G) | |
| | | (G)p.m(U)p.m(U)p.[f12r](C)p.m | | p.m(U)p.m(C)p.m(U)p.m(U)p.m | |
| | | (U)p.[fl2r](A)p.m(G)p.m(G)p.m | | (C)p.m(G)[ps].m(A)[ps].m(A)} | |
| | | (G)[ps].m(U)[ps].m(U) | | | |
| 13 | Pos11.C16.P | [5vp_una2moe](U)[ps].[fl2r](U) | 17 | {m(C)[ps].m(C)[ps].m(C)p.m(U) | 106 |
| | S.siDMPK | [ps].m(C)p.m(G)p.m(A)p.[fl2r](A) | | p.m(A)p.m(G)p.[f12r](A)p.[fl2r] | |
| | | p.m(G)p.m(A)p.m(C)p.m(A)p.m | | (A)p.[f12r](C)p.m(U)p.[rc16](G) | |
| | | (G)p.m(U)p.m(U)p.[fl2r](C)p.m | | p.m(U)p.m(C)p.m(U)p.m(U)p.m | |
| | | (U)p.[fl2r](A)p.m(G)p.m(G)p.m | | (C)p.m(G)[ps].m(A)[ps].m(A)} | |
| | | (G)[ps].m(U)[ps].m(U) | | | |
| 14 | Pos12.C16.P | [5vp_una2moe](U)[ps].[fl2r](U) | 17 | {m(C)[ps].m(C)[ps].m(C)p.m(U) | 107 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.[rc | |
| | | (G)p.m(U)p.m(U)p.[fl2r](C)p.m | | 16](U)p.m(C)p.m(U)p.m(U)p.m(C) | |
| | | (U)p.[fl2r](A)p.m(G)p.m(G)p.m | | p.m(G)[ps].m(A)[ps].m(A)} | |
| | | (G)[ps].m(U)[ps].m(U) | | | |
| 15 | Pos13.C16.P | [5vp_una2moe](U)[ps].[fl2r](U) | 17 | {m(C)[ps].m(C)[ps].m(C)p.m(U) | 108 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[fl2r](C)p.m | | p.[rc16](C)p.m(U)p.m(U)p.m(C) | |
| | | (U)p.[fl2r](A)p.m(G)p.m(G)p.m | | p.m(G)[ps].m(A)[ps].m(A)} | |
| | | (G)[ps].m(U)[ps].m(U) | | | |
| 16 | Pos14.C16.P | [5vp_una2moe](U)[ps].[fl2r](U) | 17 | {m(C)[ps].m(C)[ps].m(C)p.m(U) | 109 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[fl2r](C)p.m | | p.m(C)p.[rc16](U)p.m(U)p.m(C) | |
| | | (U)p.[fl2r](A)p.m(G)p.m(G)p.m | | p.m(G)[ps].m(A)[ps].m(A)} | |
| | | (G)[ps].m(U)[ps].m(U) | | | |
| 17 | Pos15.C16.P | [5vp_una2moe](U)[ps].[fl2r](U) | 17 | {m(C)[ps].m(C)[ps].m(C)p.m(U) | 110 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[fl2r](C)p.m | | p.m(C)p.m(U)p.[rc16](U)p.m(C) | |
| | | (U)p.[fl2r](A)p.m(G)p.m(G)p.m | | p.m(G)[ps].m(A)[ps].m(A)} | |
| | | (G)[ps].m(U)[ps].m(U) | | | |
| 18 | Pos16.C16.P | [5vp_una2moe](U)[ps].[fl2r](U) | 17 | {m(C)[ps].m(C)[ps].m(C)p.m(U) | 111 |
| | S.siDMPK | [ps].m(C)p.m(G)p.m(A)p.[f12r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[fl2r](C)p.m | | p.m(C)p.m(U)p.m(U)p.[rc16](C) | |
| | | (U)p.[fl2r](A)p.m(G)p.m(G)p.m | | p.m(G)[ps].m(A)[ps].m(A)} | |
| | | (G)[ps].m(U)[ps].m(U) | | | |
| 19 | Pos17.C16.P | [5vp_una2moe](U)[ps].[fl2r](U) | 17 | {m(C)[ps].m(C)[ps].m(C)p.m(U) | 112 |
| | S.siDMPK | [ps].m(C)p.m(G)p.m(A)p.[fl2r](A) | | p.m(A)p.m(G)p.[f12r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[fl2r](C)p.m | | p.m(C)p.m(U)p.m(U)p.m(C)p.[rc16] | |
| | | (U)p.[fl2r](A)p.m(G)p.m(G)p.m | | (G)[ps].m(A)[ps].m(A)} | |
| | | (G)[ps].m(U)[ps].m(U) | | | |
| 20 | Pos18.C16.P | [5vp_una2moe](U)[ps].[fl2r](U) | 17 | {m(C)[ps].m(C)[ps].m(C)p.m(U) | 113 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[fl2r](C)p.m | | p.m(C)p.m(U)p.m(U)p.m(C)p.m | |
| | | (U)p.[fl2r](A)p.m(G)p.m(G)p.m | | (G)[ps].[rc16](A)[ps].m(A)} | |
| | | (G)[ps].m(U)[ps].m(U) | | | |
| 21 | Pos1.C16.GS | {[rc16](U)[ps].[fl2r](U)[ps].m(C) | 18 | {m(C)[ps].m(C)[ps].m(C)p.m(U) | 114 |
| | .siDMPK | p.m(G)p.m(A)p.[fl2r](A)p.m(G | | p.m(A)p.m(G)p.[fl2r](A)p.[fl2r] | |
| | | )p.m(A)p.m(C)p.m(A)p.m(G)p. | | (A)p.[fl2r](C)p.m(U)p.m(G)p.m(U) | |
| | | m(U)p.m(U)p.[f12r](C)p.m(U)p.[fl2r] | | p.m(C)p.m(U)p.m(U)p.m(C)p. | |
| | | (A)p.m(G)p.m(G)p.m(G)[ps]. | | m(G)[ps].m(A)[ps].m(A)} | |
| | | m(U)[ps].m(U)} | | | |
| 22 | Pos2.C16.GS | {[5vp_una2moe](U)[ps].[rc16](U) | 19 | {m(C)[ps].m(C)[ps].m(C)p.m(U) | 114 |
| | .siDMPK | [ps].m(C)p.m(G)p.m(A)p.[fl2r] | | p.m(A)p.m(G)p.[fl2r](A)p.[fl2r] | |
| | | (A)p.m(G)p.m(A)p.m(C)p.m(A) | | (A)p.[fl2r](C)p.m(U)p.m(G)p.m(U) | |
| | | p.m(G)p.m(U)p.m(U)p.[fl2r](C) | | p.m(C)p.m(U)p.m(U)p.m(C)p.m | |
| | | p.m(U)p.[fl2r](A)p.m(G)p.m(G) | | (G)[ps].m(A)[ps].m(A)} | |
| | | p.m(G)[ps].m(U)[ps].m(U)} | | | |
| 23 | Pos3.C16.GS | {[5vp_una2moe](U)[ps]. [fl2r](U) | 20 | {m(C)[ps].m(C)[ps].m(C)p.m(U) | 114 |
| | .siDMPK | [ps].[rc16](C)p.m(G)p.m(A)p.[fl2r] | | p.m(A)p.m(G)p.[fl2r](A)p.[fl2r] | |
| | | (A)p.m(G)p.m(A)p.m(C)p.m | | (A)p.[fl2r](C)p.m(U)p.m(G)p.m(U) | |
| | | (A)p.m(G)p.m(U)p.m(U)p.[fl2r] | | p.m(C)p.m(U)p.m(U)p.m(C)p.m | |
| | | (C)p.m(U)p.[fl2r](A)p.m(G)p.m(G) | | (G)[ps].m(A)[ps].m(A)} | |
| | | p.m(G)[ps].m(U)[ps].m(U)} | | | |
| 24 | Pos4.C16.GS | {[5vp_una2moe](U)[ps].[fl2r](U) | 21 | {m(C)[ps].m(C)[ps].m(C)p.m(U) | 114 |
| | .siDMPK | [ps].m(C)p.[rc16](G)p.m(A)p.[fl2r] | | p.m(A)p.m(G)p.[fl2r](A)p. [fl2r] | |
| | | (A)p.m(G)p.m(A)p.m(C)p.m | | (A)p.[fl2r](C)p.m(U)p.m(G)p.m(U) | |
| | | (A)p.m(G)p.m(U)p.m(U)p.[fl2r] | | p.m(C)p.m(U)p.m(U)p.m(C)p.m | |
| | | (C)p.m(U)p.[fl2r](A)p.m(G)p.m(G) | | (G)[ps].m(A)[ps].m(A)} | |
| | | p.m(G)[ps].m(U)[ps].m(U)} | | | |
| 25 | Pos5.C16.GS | {[5vp_una2moe](U)[ps].[fl2r](U) | 22 | {m(C)[ps].m(C)[ps].m(C)p.m(U) | 114 |
| | .siDMPK | [ps].m(C)p.m(G)p.[rc16](A)p.[fl2r] | | p.m(A)p.m(G)p.[fl2r](A)p.[fl2r] | |
| | | (A)p.m(G)p.m(A)p.m(C)p.m | | (A)p.[fl2r](C)p.m(U)p.m(G)p.m(U) | |
| | | (A)p.m(G)p.m(U)p.m(U)p.[fl2r] | | p.m(C)p.m(U)p.m(U)p.m(C)p | |
| | | (C)p.m(U)p.[fl2r](A)p.m(G)p.m(G | | | |
| | | p.m(G)[ps].mU)(U)[ps].m(U)} | | | |
| 26 | Pos6.C16.GS | {[5vp_una2moe](U)[ps].[fl2r](U) | 23 | {m(C)[ps].m(C)[ps].m(C)p.m(U) | 114 |
| | .siDMPK | [ps].m(C)p.m(G)p.m(A)p.[rc16] | | p.m(A)p.m(G)p.[fl2r](A)p.[f12r] | |
| | | (A)p.m(G)p.m(A)p.m(C)p.m(A) | | (A)p.[fl2r](C)p.m(U)p.m(G)p.m(U) | |
| | | p.m(G)p.m(U)p.m(U)p.[fl2r](C) | | p.m(C)p.m(U)p.m(U)p.m(C)p. | |
| | | p.m(U)p.[fl2r](A)p.m(G)p.m(G) | | m(G)[ps].m(A)[ps].m(A)} | |
| | | p.m(G)[ps].m(U)[ps].m(U)} | | | |
| 27 | Pos7.C16.GS | {[5vp_una2moe](U)[ps].[fl2r](U) | 24 | {m(C)[ps].m(C)[ps].m(C)p.m(U) | 114 |
| | .siDMPK | [ps].m(C)p.m(G)p.m(A)p.[fl2r](A) | | p.m(A)p.m(G)p.[fl2r](A)p.[fl2r] | |
| | | p.[rc16](G)p.m(A)p.m(C)p.m(A) | | (A)p.[fl2r](C)p.m(U)p.m(G)p.m(U) | |
| | | p.m(G)p.m(U)p.m(U)p.[fl2r](C) | | p.m(C)p.m(U)p.m(U)p.m(C)p | |
| | | p.m(U)p.[fl2r](A)p.m(G)p.m(G) | | | |
| | | p.m(G)[ps].m(U)[ps].m(U)} | | | |
| 28 | Pos8.C16.GS | {[5vp_una2moe](U)[ps].[fl2r](U) | 25 | {m(C)[ps].m(C)[ps].m(C)p.m(U) | 114 |
| | .siDMPK | [ps].m(C)p.m(G)p.m(A)p.[fl2r](A) | | p.m(A)p.m(G)p.[fl2r](A)p.[fl2r] | |
| | | p.m(G)p.[rc16](A)p.m(C)p.m(A) | | (A)p.[fl2r](C)p.m(U)p.m(G)p.m(U) | |
| | | p.m(G)p.m(U)p.m(U)p.[fl2r](C) | | p.m(C)p.m(U)p.m(U)p.m(C)p.m | |
| | | p.m(U)p.[fl2r](A)p.m(G)p.m(G) | | (G)[ps].m(A)[ps].m(A)} | |
| | | p.m(G)[ps].m(U)[ps].m(U)} | | | |
| 29 | Pos9.C16.GS | {[5vp_una2moe](U)[ps].[fl2r](U) | 26 | {m(C)[ps].m(C)[ps].m(C)p.m(U) | 114 |
| | .siDMPK | [ps].m(C)p.m(G)p.m(A)p.[fl2r](A) | | p.m(A)p.m(G)p.[fl2r](A)p.[fl2r] | |
| | | p.m(G)p.m(A)p.[rc16](C)p.m(A) | | (A)p.[fl2r](C)p.m(U)p.m(G)p.m(U) | |
| | | p.m(G)p.m(U)p.m(U)p.[fl2r](C) | | p.m(C)p.m(U)p.m(U)p.m(C)p.m | |
| | | p.m(U)p.[fl2r](A)p.m(G)p.m(G) | | (G)[ps].m(A)[ps].m(A)} | |
| | | p.m(G)[ps].m(U)[ps].m(U)} | | | |
| 30 | Pos10.C16.G | {[5vp_una2moe](U)[ps].[fl2r](U) | 27 | {m(C)[ps].m(C)[ps].m(C)p.m(U) | 114 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.[rc16](A) | | (A)p.[fl2r](C)p.m(U)p.m(G)p.m(U) | |
| | | p.m(G)p.m(U)p.m(U)p.[fl2r](C) | | p.m(C)p.m(U)p.m(U)p.m(C)p.m | |
| | | p.m(U)p.[fl2r](A)p.m(G)p.m(G) | | (G)[ps].m(A)[ps].m(A)} | |
| | | p.m(G)[ps].m(U)[ps].m(U)} | | | |
| 31 | Pos11.C16.G | {[5vp_una2moe](U)[ps].[f12r](U) | 28 | {m(C)[ps].m(C)[ps].m(C)p.m(U) | 114 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.[rc16] | | (A)p.[fl2r](C)p.m(U)p.m(G)p.m(U) | |
| | | (G)p.m(U)p.m(U)p.[fl2r](C) | | p.m(C)p.m(U)p.m(U)p.m(C)p.m | |
| | | p.m(U)p.[fl2r](A)p.m(G)p.m(G) | | (G)[ps].m(A)[ps].m(A)} | |
| | | p.m(G)[ps].m(U)[ps].m(U)} | | | |
| 32 | Pos12.C16.G | {[5vp_una2moe](U)[ps]. [f12r](U) | 29 | {m(C)[ps].m(C)[ps].m(C)p.m(U) | 114 |
| | S.siDMPK | [ps].m(C)p.m(G)p.m(A)p.[f12r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.[rc16](U)p.m(U)p.[fl2r](C) | | p.m(C)p.m(U)p.m(U)p.m(C)p. | |
| | | p.m(U)p.[fl2r](A)p.m(G)p.m(G) | | m(G)[ps].m(A)[ps].m(A)} | |
| | | p.m(G)[ps].m(U)[ps].m(U)} | | | |
| 33 | Pos13.C16.G | {[5vp_una2moe](U)[ps]. [fl2r](U) | 30 | {m(C)[ps].m(C)[ps].m(C)p.m(U) | 114 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.[rc16](U)p.[fl2r](C) | | p.m(C)p.m(U)p.m(U)p.m(C)p.m | |
| | | p.m(U)p.[fl2r](A)p.m(G)p.m(G) | | (G)[ps].m(A)[ps].m(A)} | |
| | | p.m(G)[ps].m(U)[ps].m(U)} | | | |
| 34 | Pos14.C16.G | {[5vp_una2moe](U)[ps].[f12r](U) | 31 | {m(C)[ps].m(C)[ps].m(C)p.m(U | 114 |
| | S.siDMPK | [ps].m(C)p.m(G)p.m(A)p.[fl2r](A) | | )p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[rc16](C)p.m | | p.m(C)p.m(U)p.m(U)p.m(C)p.m | |
| | | (U)p.[fl2r](A)p.m(G)p.m(G)p.m | | (G)[ps].m(A)[ps].m(A)} | |
| | | (G)[ps].m(U)[ps].m(U)} | | | |
| 35 | Pos15.C16.G | {[5vp_una2moe](U)[ps]. [fl2r](U) | 32 | {m(C)[ps].m(C)[ps].m(C)p.m(U) | 114 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[fl2r](C)p.[rc16] | | p.m(C)p.m(U)p.m(U)p.m(C)p.m | |
| | | (U)p.[fl2r](A)p.m(G)p.m(G) | | (G)[ps].m(A)[ps].m(A)} | |
| | | p.m(G)[ps].m(U)[ps].m(U)} | | | |
| 36 | Pos16.C16.G | {[5vp_una2moe](U)[ps].[fl2r](U) | 33 | {m(C)[ps].m(C)[ps].m(C)p.m(U) | 114 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[fl2r](C)p.m | | p.m(C)p.m(U)p.m(U)p.m(C)p.m | |
| | | (U)p.[rc16](A)p.m(G)p.m(G)p.m | | (G)[ps].m(A)[ps].m(A)} | |
| | | (G)[ps].m(U)[ps].m(U)} | | | |
| 37 | Pos17.C16.G | {[5vp_una2moe](U)[ps].[fl2r](U) | 34 | {m(C)[ps].m(C)[ps].m(C)p.m(U) | 114 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[f12r](C)p.m(U)p.m(G)p.m(U) | |
| | | (G)p.m(U)p.m(U)p.[fl2r](C)p.m | | p.m(C)p.m(U)p.m(U)p.m(C)p.m | |
| | | (U)p.[fl2r](A)p.[rc16](G)p.m(G) | | (G)[ps].m(A)[ps].m(A)} | |
| | | p.m(G)[ps].m(U)[ps].m(U)} | | | |
| 38 | Pos18.C16.G | {[5vp_una2moe](U)[ps]. [fl2r](U) | 35 | {m(C)[ps].m(C)[ps].m(C)p.m(U) | 114 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[fl2r](C)p.m | | p.m(C)p.m(U)p.m(U)p.m(C)p.m | |
| | | (U)p.[fl2r](A)p.m(G)p.[rc16](G) | | (G)[ps].m(A)[ps].m(A)} | |
| | | p.m(G)[ps].m(U)[ps].m(U)} | | | |
| 39 | Pos19.C16.G | {[5vp_una2moe](U)[ps]. [f12r](U) | 36 | {m(C)[ps].m(C)[ps].m(C)p.m(U) | 114 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[f12r](C)p.m | | p.m(C)p.m(U)p.m(U)p.m(C)p.m | |
| | | (U)p.[fl2r](A)p.m(G)p.m(G)p.[rc16] | | (G)[ps].m(A)[ps].m(A)} | |
| | | (G)[ps].m(U)[ps].m(U)} | | | |
| 40 | Pos20.C16.G | {[5vp_una2moe](U)[ps].[fl2r](U) | 37 | {m(C)[ps].m(C)[ps].m(C)p.m(U) | 114 |
| | S.siDMPK | [ps].m(C)p.m(G)p.m(A)p.[fl2r](A) | | p.m(A)p.m(G)p.[fl2r](A)p.[fl2r] | |
| | | p.m(G)p.m(A)p.m(C)p.m(A)p.m | | (A)p.[fl2r](C)p.m(U)p.m(G)p.m(U) | |
| | | (G)p.m(U)p.m(U)p.[fl2r](C)p.m | | p.m(C)p.m(U)p.m(U)p.m(C)p.m | |
| | | (U)p.[fl2r](A)p.m(G)p.m(G)p.m | | (G)[ps].m(A)[ps].m(A)} | |
| | | (G)[ps].[rc 16](U)[ps].m(U)} | | | |
| 41 | Pos21.C16.G | {[5vpuna2moc[(U)[ps].[fl2r](U)[ps]. | 38 | {m(C)[s].m(C)[ps].m(C)p.m(U) | |
| | S.siDMPK | m(C)p.m(G)p.m(A)p.[fl2](A)p.m | | p.m(A)p.m(G)p.[fl2r](A)p.[fl2r] | |
| | | (G)p.m(A)p.m(C)p.m(A)p.m(G) | | (A)p.[fl2r](C)p.m(U)p.m(G)p.m(U) | |
| | | p.m(U)p.m(U)p.[f12](C)p.m(U)p.[fl2] | | p.m(C)p.m(U)p.m(U)p.m(C)p.m | |
| | | (A)p.m(G)p.m(G)p.m(G)[ps].m | | (G)[ps].m(A)[ps].m(A)} | |
| | | (U)[ps].[rc16](U)} | | | |

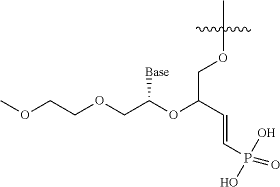

5vp_una2moe 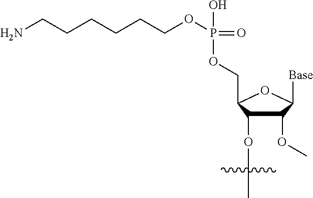
5am6_p_m 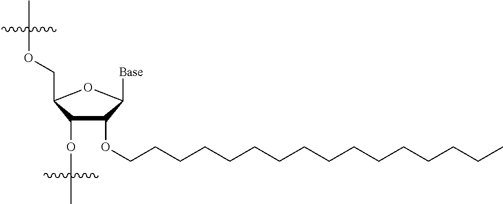
rc16

In Table 77, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage; [rc16] indicates 2′O—C16 Phosphoramidite.

### Conjugate Synthesis and Characterization

The CD71 Fab-siRNA conjugates were made, purified and characterized as described in Example 3. All conjugates were made through cysteine conjugation with a SMCC linker. Conjugates were characterized chromatographically as described in Table 78.

TABLE 78
siRNA Sequences used in Example 26

| | | | SEQ | | SEQ |
|---|---|---|---|---|---|
| Compound | | | ID | | ID |
| No. | siRNA Ref. | Guide Sequence | NO: | siRNA Passenger Sequence | NO: |
| | GS.siDMPK | (C)p.m(G)p.m(A)p.[fl2r](A)p.m | | p.m(A)p.m(G)p.[f12r](A)p.[fl2r] | |
| | | (G)p.m(A)p.m(C)p.m(A)p.m(G) | | (A)p.[fl2r](C)p.m(U)p.m(G)p.m(U) | |
| | | p.m(U)p.m(U)p.[fl2r](C)p.m(U)p.[rc16] | | p.m(C)p.m(U)p.m(U)p.m(C)p.m | |
| | | (A)p.m(G)p.m(G)p.m(G)[ps].m | | (G)[ps].m(A)[ps].m(A)} | |
| | | (U)[ps].m(U)} | | | |
| 37 | Pos17.C16. | {[5vp_una2moe](U)[ps].[fl2r](U)[ps].m | 34 | {m(C)[ps].m(C)[ps].m(C)p.m(U) | 114 |
| | GS.siDMPK | (C)p.m(G)p.m(A)p.[fl2r](A)p.m | | p.m(A)p.m(G)p.[fl2r](A)p.[fl2r] | |
| | | (G)p.m(A)p.m(C)p.m(A)p.m(G) | | (A)p.[fl2r](C)p.m(U)p.m(G)p.m(U) | |
| | | p.m(U)p.m(U)p.[fl2r](C)p.m(U)p.[fl2r] | | p.m(C)p.m(U)p.m(U)p.m(C)p.m | |
| | | (A)p.[rc16](G)p.m(G)p.m(G)[ps].m | | (G)[ps].m(A)[ps].m(A)} | |
| | | (U)[ps].m(U)} | | | |
| 38 | Pos18.C16. | {[5vp_una2moe](U)[ps].[fl2r](U)[ps].m | 35 | {m(C)[ps].m(C)[ps].m(C)p.m(U) | 114 |
| | GS.siDMPK | (C)p.m(G)p.m(A)p.[fl2r](A)p.m | | p.m(A)p.m(G)p.[fl2r](A)p.[fl2r] | |
| | | (G)p.m(A)p.m(C)p.m(A)p.m(G) | | (A)p.[fl2r](C)p.m(U)p.m(G)p.m(U) | |
| | | p.m(U)p.m(U)p.[fl2r](C)p.m(U)p.[fl2r] | | p.m(C)p.m(U)p.m(U)p.m(C)p.m | |
| | | (A)p.m(G)p.[rc16](G)p.m(G)[ps]. | | (G)[ps].m(A)[ps].m(A)} | |
| | | m(U)[ps].m(U)} | | | |
| | | fl2r[(A)p.m(G)p.[rc16](G)p.m(G)[ps].m | | | |
| | | ps].m(U)[ps].m(U)} | | | |
| 39 | Pos19.C16. | {[5vp_una2moe](U)[ps].[fl2r](U)[ps].m | 36 | {m(C)[ps].m(C)[ps].m(C)p.m(U) | 114 |
| | GS.siDMPK | (C)p.m(G)p.m(A)p.[fl2r](A)p.m | | p.m(A)p.m(G)p.[fl2r](A)p.[fl2r] | |
| | | (G)p.m(A)p.m(C)p.m(A)p.m(G) | | (A)p.[fl2r](C)p.m(U)p.m(G)p.m(U) | |
| | | p.m(U)p.m(U)p.[fl2r](C)p.m(U)p.[fl2r] | | p.m(C)p.m(U)p.m(U)p.m(C)p.m | |
| | | (A)p.m(G)p.m(G)p.[rc16](G)[ps].m | | (G)[ps].m(A)[ps].m(A)} | |
| | | (U)[ps].m(U)} | | | |
| 40 | Pos20.C16. | {[5vp_una2moe](U)[ps]. [f12r](U)[ps].m | 37 | {m(C)[ps].m(C)[ps].m(C)p.m(U) | 114 |
| | GS.siDMPK | (C)p.m(G)p.m(A)p.[fl2r](A)p.m(G) | | p.m(A)p.m(G)p.[fl2r](A)p.[fl2r] | |
| | | p.m(A)p.m(C)p.m(A)p.m(G) | | (A)p.[fl2r](C)p.m(U)p.m(G)p.m(U) | |
| | | p.m(U)p.m(U)p.[fl2r](C)p.m(U)p.[fl2r] | | p.m(C)p.m(U)p.m(U)p.m(C)p.m | |
| | | (A)p.m(G)p.m(G)p.m(G)[ps].[rc16] | | (G)[ps].m(A)[ps].m(A)} | |
| | | (U)[ps].m(U)} | | | |
| 41 | Pos21.C16. | {[5vp_una2moe](U)[ps].[fl2r](U)[ps].m | 38 | {m(C)[ps].m(C)[ps].m(C)p.m(U) | 114 |
| | GS.siDMPK | (C)p.m(G)p.m(A)p.[fl2r](A)p.m | | p.m(A)p.m(G)p.[fl2r](A)p.[fl2r] | |
| | | (G)p.m(A)p.m(C)p.m(A)p.m(G) | | (A)p.[fl2r](C)p.m(U)p.m(G)p.m(U) | |
| | | p.m(U)p.m(U)p.[fl2r](C)p.m(U)p.[fl2r] | | p.m(C)p.m(U)p.m(U)p.m(C)p.m | |
| | | (A)p.m(G)p.m(G)p.m(G)[ps].m | | (G)[ps].m(A)[ps].m(A)} | |
| | | (U)[ps].[rc16](U)} | | | |

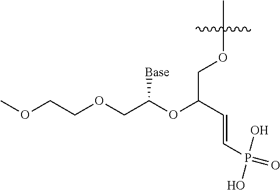

5vp_una2moe 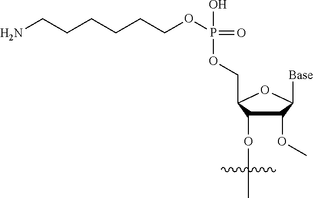
5am6_p_m 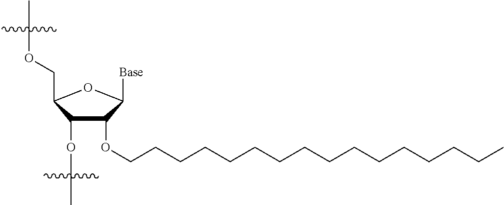
rc16

In Table 78, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage; [rc16] indicates 2′O—C16 Phosphoramidite

TABLE 79
HPLC Retention Time (RT) in Minutes

| Group(s) | Conjugate | RT, HIC Method-# |
|---|---|---|
| 1 | Pos16.C16.GS.siDMPK FabOC DAR1 | 21.495 |
| 2 | Pos16.C16.GS.siDMPK FabOC DAR2 | 27.332 |
| 3 | Pos17.C16.GS.siDMPK FabOC DAR1 | 22.167 |
| 4 | Pos17.C16.GS.siDMPK FabOC DAR2 | 26.491 |
| 5 | Pos18.C16.GS.siDMPK FabOC DAR1 | 19.924 |
| 6 | Pos18.C16.GS.siDMPK FabOC DAR2 | 23.590 |
| 7 | Pos19.C16.GS.siDMPK FabOC DAR1 | 17.159 |
| 8 | Pos19.C16.GS.siDMPK FabOC DAR2 | 19.030 |
| 9 | Pos20.C16.GS.siDMPK FabOC DAR1 | 16.852 |
| 10 | Pos20.C16.GS.siDMPK FabOC DAR2 | 19.289 |
| 11 | Pos21.C16.GS.siDMPK FabOC DAR1 | 18.075 |
| 12 | Pos21.C16.GS.siDMPK FabOC DAR2 | 20.139 |

### Results

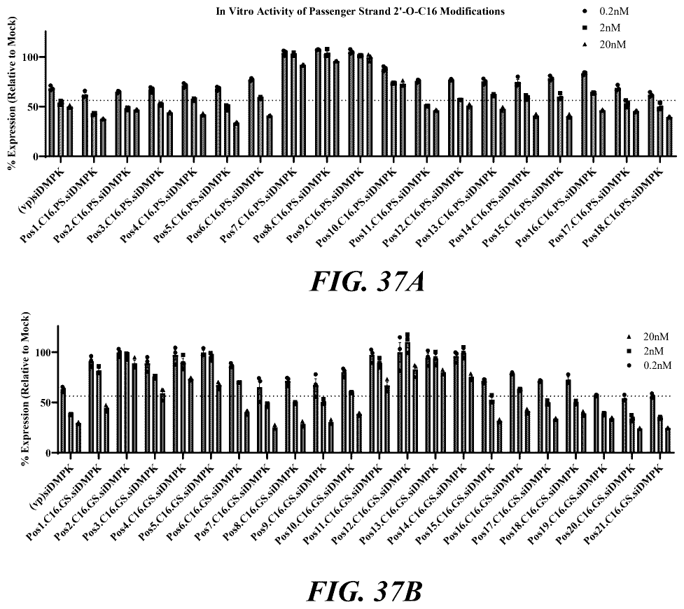

DMPK siRNA sequences containing C16 modification at every passenger strand and guide strand position were assessed for their ability to knockdown DMPK mRNA expression levels in a luciferase-reporter assay in HEK293 cells. As shown in FIGS. 37A-37B, DMPK siRNA with C16 modification at positions 7-9 nucleotide on the passenger strand had lower knockdown DMPK mRNA expression levels than control DMPK siRNA and DMPK siRNA with C16 modification at other positions of the passenger strand. In addition, DMPK siRNA with C16 modification at positions 1-6 and 11-15 nucleotide on the guide strand had lower knockdown DMPK mRNA expression levels than the control siRNA and siRNA with C16 modification at other positions of the guide strand. Overall, the position of the C16 modification on the passenger and guide strand of the siRNA affect the activity of the siRNA.

Finally, DAR1 and DAR2 FabOCs containing a 2′-O—C16 modification of positions 16, 17, 18, 19, 20, and 21 nucleotide of the guide strand were synthesized and isolated by HPLC as shown in Table 79.

### Example 27: In Vitro Evaluation of Degradable/Cleavable Lipid-modified siRNAs

In Vitro Evaluation of siRNA Efficacy Luciferase-reporter assay in HEK293 cells HEK 293 cells were engineered to express DMPK within the psiCHECK™-2 vector (Promega). Cells were grown in DMEM supplemented with 10% FBS (Corning). Cells were seeded in triplicate at 20,000 cells/well in 96-well tissue culture plates without antibiotics 24 h prior to transfection. On the day of transfection, siRNAs were formulated with RNAiMax (Life Technologies, Waltham, MA, USA) and OptiMEM (Life Technologies, Waltham, MA, USA), transfection was performed following manufacturer's instructions. 48 h after transfection, firefly and Renilla luciferase activities were measured using the Dual-Glo® Luciferase Assay System (Promega, Madison, WI, USA) according to manufacturer's instructions. Luminescence signal was read on Infinite® M200 Pro plate reader (Tecan, Mannedorf, Switzerland). The relative Renilla/firefly luciferase ratio was calculated by first subtracting the background from the luciferase activity measurements (no-reporter transfection controls used as background reference) and then generating the relative ratio to no-siRNA transfection control.

TABLE 80
siRNA Sequences used in Example 27

| | | | SEQ | | SEQ |
|---|---|---|---|---|---|
| Compound | siRNA | | ID | | ID |
| No. | Ref. | siRNA Guide Sequence | NO: | siRNA Passenger Sequence | NO: |
| 1 | (vp)siDM | [5vp_una2moe](U)[ps].[fl2r](U)[ps].m | 17 | [5am6_p_m](C)[ps].m(C)[ps].m(C) | 115 |
| | PK | (C)p.m(G)p.m(A)p.[fl2r](A)p.m(G) | | p.m(U)p.m(A)p.m(G)p.[fl2r](A) | |
| | | p.m(A)p.m(C)p.m(A)p.m(G)p.m(U)p.m | | p.[fl2r](A)p.[fl2r](C)p.m(U)p.m(G) | |
| | | (U)p.[fl2r](C)p.m(U)p.[fl2r](A)p.m(G) | | p.m(U)p.m(C)p.m(U)p.m(U)p.m | |
| | | p.m(G)p.m(G)[ps].m(U)[ps].m(U) | | (C)p.m(G)[ps].m(A)[ps].m(A) | |
| 2 | Pos3.C22. | [5vp_una2moe](U)[ps].[fl2r](U)[ps].m | 17 | [5am6_p_m](C)[ps].m(C)[ps].[rc22] | 116 |
| | siDMPK | (C)p.m(G)p.m(A)p.[fl2r](A)p.m(G) | | (C)p.m(U)p.m(A)p.m(G)p.[fl2r] | |
| | | p.m(A)p.m(C)p.m(A)p.m(G)p.m(U)p.m | | (A)p.[fl2r](A)p.[fl2r](C)p.m(U) | |
| | | (U)p.[fl2r](C)p.m(U)p.[fl2r](A)p.m(G) | | p.m(G)p.m(U)p.m(C)p.m(U)p.m(U) | |
| | | p.m(G)p.m(G)[ps].m(U)[ps].m(U) | | p.m(C)p.m(G)[ps].m(A)[ps].m(A) | |
| 3 | Standard | [5vp_una2moe](U)[ps].[fl2r](U)[ps].m | 17 | [5am6_p_rc16](C)[ps].m(C)[ps].m | 117 |
| | 7f.8s with | (C)p.m(G)p.m(A)p.[fl2r](A)p.m(G) | | (C)p.m(U)p.m(A)p.m(G)p.[fl2r] | |
| | PS pos. 1 | p.m(A)p.m(C)p.m(A)p.m(G)p.m(U)p.m | | (A)p.[fl2r](A)p.[fl2r](C)p.m(U)p.m | |
| | lipid | (U)p.[fl2r](C)p.m(U)p.[fl2r](A)p.m(G) | | (G)p.m(U)p.m(C)p.m(U)p.m(U) | |
| | | p.m(G)p.m(G)[ps].m(U)[ps].m(U) | | p.m(C)p.m(G)[ps].m(A)[ps].m(A) | |
| 4 | Non-lipid | [5vp_una2moe](U)[ps].[fl2r](U)[ps].m | 39 | [5am6_p_m](C)p.m(C)[ps].m(C) | 118 |
| | GS.4f.5S// | (C)p.m(G)p.m(A)p.[fl2r](A)p.m(G) | | p.m(U)p.m(A)p.m(G)p.[fl2r](A)p.[fl2r] | |
| | PS.3f.3s | p.m(A)p.m(C)p.m(A)p.m(G)p.m(U)p.m | | (A)p.[fl2r](C)p.m(U)p.m(G) | |
| | | (U)p.[fl2r](C)p.m(U)p.[fl2r](A)p.m(G) | | p.m(U)p.m(C)p.m(U)p.m(U)p.m(C) | |
| | | p.m(G)[ps].m(G)[ps].m(U)[ps].m(U) | | p.m(G)[ps].m(A)[ps].m(A) | |
| 5 | Non-lipid | [5vp_una2moe](U)[ps].[fl2r](U)[ps].m | 39 | [5am6_p_m](C)[ps].m(C)p.m(U) | 119 |
| | GS.4f.5S// | (C)p.m(G)p.m(A)p.[fl2r](A)p.m(G) | | p.m(A)p.m(G)p.[fl2r](A)p.[fl2r](A) | |
| | PS.3f.3s | p.m(A)p.m(C)p.m(A)p.m(G)p.m(U)p.m | | p.[fl2r](C)p.m(U)p.m(G)p.m(U) | |
| | with n-1 | (U)p.[f12r](C)p.m(U)p.[fl2r](A)p.m(G) | | p.m(C)p.m(U)p.m(U)p.m(C)p.m(G) | |
| | | p.m(G)[ps].m(G)[ps].m(U)[ps].m(U) | | [ps].m(A)[ps].m(A) | |
| 6 | PS pos.1 | [5vp_una2moe](U)[ps].[fl2r](U)[ps].m | 39 | [5am6_p_rc16](C)p.m(C)[ps].m(C) | 120 |
| | lipid with | (C)p.m(G)p.m(A)p.[fl2r](A)p.m(G) | | p.m(U)p.m(A)p.m(G)p.[fl2r](A) | |
| | GS.4f.5S// | p.m(A)p.m(C)p.m(A)p.m(G)p.m(U)p.m | | p.[fl2r](A)p.[fl2r](C)p.m(U)p.m(G) | |
| | PS.3f.3S | (U)p.[fl2r](C)p.m(U)p.[fl2r](A)p.m(G) | | p.m(U)p.m(C)p.m(U)p.m(U)p.m | |
| | | p.m(G)[ps].m(G)[ps].m(U)[ps].m(U) | | (C)p.m(G)[ps].m(A)[ps].m(A) | |
| 7 | 20/21-mer | [5vp_una2moe](U)[ps].[fl2r](U)[ps].m | 17 | [5am6_p_m](A)[ps].m(C)[ps].m(C) | 121 |
| | 7f.8s | (C)p.m(G)p.m(A)p.[fl2r](A)p.m(G) | | p.m(C)p.m(U)p.m(A)p.m(G)p.[fl2r] | |
| | stable | p.m(A)p.m(C)p.m(A)p.m(G)p.m(U)p.m | | (A)p.[fl2r](A)p.[fl2r](C)p.m(U) | |
| | duplex, | (U)p.[fl2r](C)p.m(U)p.[fl2r](A)p.m(G) | | p.m(G)p.m(U)p.m(C)p.m(U)p.m | |
| | non-lipid | p.m(G)p.m(G)[ps].m(U)[ps].m(U) | | (U)p.m(C)p.m(G)[ps].m(A)[ps].m | |
| | | | | (A) | |
| 8 | 20/21-mer | [5vp_una2moe](U)[ps].[fl2r](U)[ps]. | 17 | [5am6_p_rc16](A)[ps].m(C)[ps].m | 122 |
| | 7f.8s | m(C)p.m(G)p.m(A)p.[fl2r](A)p.m(G) | | (C)p.m(C)p.m(U)p.m(A)p.m(G) | |
| | stable | p.m(A)p.m(C)p.m(A)p.m(G)p.m(U)p.m | | p.[fl2r](A)p.[fl2r](A)p.[fl2r](C)p. | |
| | duplex, | (U)p.[fl2r](C)p.m(U)p.[fl2r](A)p.m(G) | | m(U)p.m(G)p.m(U)p.m(C)p.m(U) | |
| | lipid | p.m(G)p.m(G)[ps].m(U)[ps].m(U) | | p.m(U)p.m(C)p.m(G)[ps].m(A)[ | |
| | | | | ps].m(A) | |
| 9 | 20/21-mer | [5vp_una2moe](U)[ps].[fl2r](U)[ps].m | 17 | [5am6_p_m](A)p.m(C)[ps].m(C)[ps].m | 123 |
| | 7f.8s | (C)p.m(G)p.m(A)p.[fl2r](A)p.m(G) | | (C)p.m(U)p.m(A)p.m(G)p.[fl2r] | |
| | degradable | p.m(A)p.m(C)p.m(A)p.m(G)p.m(U)p.m | | (A)p.[fl2r](A)p.[fl2r](C)p.m(U) | |
| | duplex, | (U)p.[fl2r](C)p.m(U)p.[fl2r](A)p.m(G) | | p.m(G)p.m(U)p.m(C)p.m(U)p.m | |
| | non-lipid | p.m(G)p.m(G)[ps].m(U)[ps].m(U) | | (U)p.m(C)p.m(G)[ps].m(A)[ps].m | |
| | | | | (A) | |
| 10 | 20/21-mer | [5vp_una2moe](U)[ps].[fl2r](U)[ps].m | 17 | [5am6_p_rc16](A)p.m(C)[ps].m(C) | 124 |
| | 7f.8s | (C)p.m(G)p.m(A)p.[fl2r](A)p.m(G) | | [ps].m(C)p.m(U)p.m(A)p.m(G) | |
| | degradable | p.m(A)p.m(C)p.m(A)p.m(G)p.m(U)p.m | | p.[fl2r](A)p.[fl2r](A)p.[fl2r](C)p.m | |
| | duplex, | (U)p.[fl2r](C)p.m(U)p.[fl2r](A)p.m(G) | | (U)p.m(G)p.m(U)p.m(C)p.m(U) | |
| | lipid | p.m(G)p.m(G)[ps].m(U)[ps].m(U) | | p.m(U)p.m(C)p.m(G)[ps].m(A)[ps].m | |
| | | | | (A) | |
| 11 | 20/21-mer | [5vp_una2moe](U)[ps].[fl2r](U)[ps].m | 17 | {[5am6_p_m](U)[ps].m(C)[ps].m | 125 |
| | 7f.8s | (C)p.m(G)p.m(A)p.[fl2r](A)p.m(G) | | (C)p.m(C)p.m(U)p.m(A)p.m(G)p.[fl2r] | |
| | stable | p.m(A)p.m(C)p.m(A)p.m(G)p.m(U)p. | | (A)p.[fl2r](A)p.[fl2r](C)p.m | |
| | overhang | m(U)p.[fl2r](C)p.m(U)p.[fl2r](A)p.m(G) | | (U)p.m(G)p.m(U)p.m(C)p.m(U)p.m | |
| | MM | p.m(G)p.m(G)[ps].m(U)[ps].m(U) | | (U)p.m(C)p.m(G)[ps].m(A)[ps].m | |
| | duplex, | | | (A)} | |
| | non-lipid | | | | |
| 12 | 20/21-mer | [5vp_una2moe](U)[ps].[fl2r](U)[ps].m | 17 | [5am6_p_rc16](U)[ps].m(C)[ps].m | 126 |
| | 7f.8s | (C)p.m(G)p.m(A)p.[fl2r](A)p.m(G) | | (C)p.m(C)p.m(U)p.m(A)p.m(G) | |
| | stable | p.m(A)p.m(C)p.m(A)p.m(G)p.m(U)p.m | | p.[fl2r](A)p.[fl2r](A)p.[fl2r](C)p.m | |
| | overhang | (U)p.[fl2r](C)p.m(U)p.[fl2r](A)p.m(G) | | (U)p.m(G)p.m(U)p.m(C)p.m(U) | |
| | MM | p.m(G)p.m(G)[ps].m(U)[ps].m(U) | | p.m(U)p.m(C)p.m(G)[ps].m(A)[ps]. | |
| | duplex, | | | m(A) | |
| | lipid | | | | |
| 13 | 20/21-mer | [5vp_una2moe](U)[ps].[fl2r](U)[ps]. | 17 | [5am6_p_m](U)p.m(C)[ps].m(C)[ps].m | 127 |
| | 7f.8s | m(C)p.m(G)p.m(A)p.[fl2r](A)p.m(G) | | (C)p.m(U)p.m(A)p.m(G)p.[fl2r] | |
| | degradable | p.m(A)p.m(C)p.m(A)p.m(G)p.m(U)p.m | | (A)p.[fl2r](A)p.[fl2r](C)p.m(U) | |
| | overhang | (U)p.[fl2r](C)p.m(U)p.[fl2r](A)p.m(G) | | p.m(G)p.m(U)p.m(C)p.m(U)p.m | |
| | MM | p.m(G)p.m(G)[ps].m(U)[ps].m(U) | | (U)p.m(C)p.m(G)[ps].m(A)[ps].m | |
| | duplex, | | | (A) | |
| | non-lipid | | | | |
| 14 | 20/21-mer | [5vp_una2moe](U)[ps].[fl2r](U)[ps]. | 17 | [5am6_p_rc16](U)p.m(C)[ps].m(C) | 128 |
| | 7f.8s | m(C)p.m(G)p.m(A)p.[fl2r](A)p.m(G) | | [ps].m(C)p.m(U)p.m(A)p.m(G) | |
| | degradable | p.m(A)p.m(C)p.m(A)p.m(G)p.m(U)p. | | p.[fl2r](A)p.[fl2r](A)p.[fl2r](C)p.m | |
| | overhang | m(U)p.[fl2r](C)p.m(U)p.[fl2r](A)p.m( | | (U)p.m(G)p.m(U)p.m(C)p.m(U) | |
| | MM | G)p.m(G)p.m(G)[ps].m(U)[ps].m(U) | | p.m(U)p.m(C)p.m(G)[ps].m(A)[ps].m | |
| | duplex, | | | (A) | |
| | lipid | | | | |
| 15 | Non-lipid | [5vp_una2moe](U)[ps].[fl2r](U)[ps].m | 40 | [5am6_p_m](C)[ps].m(C)[ps].m(C) | 115 |
| | 7f.7s with | (C)p.m(G)p.m(A)p.[fl2r](A)p.m(G) | | p.m(U)p.m(A)p.m(G)p.[fl2r](A) | |
| | degradable | p.m(A)p.m(C)p.m(A)p.m(G)p.m(U)p.m | | p.[fl2r](A)p.[fl2r](C)p.m(U)p.m(G) | |
| | 3′-GS | (U)p.[fl2r](C)p.m(U)p.[fl2r](A)p.m(G) | | p.m(U)p.m(C)p.m(U)p.m(U)p.m | |
| | | p.m(G)p.m(G)[ps].m(U)p.m(U) | | (C)p.m(G)[ps].m(A)[ps].m(A) | |
| 16 | GS | [5vp_una2moe](U)[ps].[fl2r](U)[ps].m | 41 | [5am6_p_m](C)[ps].m(C)[ps].m(C) | 115 |
| | pos21.C16 | (C)p.m(G)p.m(A)p.[fl2r](A)p.m(G) | | p.m(U)p.m(A)p.m(G)p.[fl2r](A) | |
| | 7f.7s with | p.m(A)p.m(C)p.m(A)p.m(G)p.m(U)p.m | | p.[fl2r](A)p.[fl2r](C)p.m(U)p.m(G) | |
| | degradable | (U)p.[fl2r](C)p.m(U)p.[fl2r](A)p.m(G) | | p.m(U)p.m(C)p.m(U)p.m(U)p.m | |
| | 3′-GS | p.m(G)p.m(G)[ps].m(U)p.[rc16](U) | | (C)p.m(G)[ps].m(A)[ps].m(A) | |
| 17 | GS | [5vp_una2moe](U)[ps].[fl2r](U)[ps]. | 42 | [5am6_p_m](C)[ps].m(C)[ps].m(C) | 115 |
| | pos21.C16 | m(C)p.m(G)p.m(A)p.[fl2r](A)p.m(G) | | p.m(U)p.m(A)p.m(G)p.[fl2r](A) | |
| | 7f.8s with | p.m(A)p.m(C)p.m(A)p.m(G)p.m(U)p. | | p.[fl2r](A)p.[fl2r](C)p.m(U)p.m(G) | |
| | stable 3′- | m(U)p.[fl2r](C)p.m(U)p.[fl2r](A)p.m(G) | | p.m(U)p.m(C)p.m(U)p.m(U)p.m | |
| | GS | p.m(G)p.m(G)[ps].m(U)[ps].[rc16](U) | | (C)p.m(G)[ps].m(A)[ps].m(A) | |
| 18 | GS 7f.7s | [5vp_una2moe](U)[ps].[fl2r](U)[ps].m | 43 | [5am6_p_m](C)[ps].m(C)[ps].m(C) | 115 |
| | with | (C)p.m(G)p.m(A)p.[fl2r](A)p.m(G) | | p.m(U)p.m(A)p.m(G)p.[fl2r](A) | |
| | degradable | p.m(A)p.m(C)p.m(A)p.m(G)p.m(U)p.m | | p.[fl2r](A)p.[fl2r](C)p.m(U)p.m(G) | |
| | 3′-GS | (U)p.[fl2r](C)p.m(U)p.[fl2r](A)p.m(G) | | p.m(U)p.m(C)p.m(U)p.m(U)p.m | |
| | with n-1 | p.m(G)p.m(G)[ps].m(U) | | (C)p.m(G)[ps].m(A)[ps].m(A) | |
| 19 | 3′_(dT)2 | [5vp_una2moe](U)[ps].[fl2r](U)[ps].m | 17 | m(C)[ps].m(C)[ps].m(C)p.m(C)p.m | 129 |
| | 7f8s | (C)p.m(G)p.m(A)p.[fl2r](A)p.m(G) | | (U)p.m(A)p.m(G)p.[fl2r](A)p.[fl2r] | |
| | | p.m(A)p.m(C)p.m(A)p.m(G)p.m(U)p.m | | (A)p.[fl2r](C)p.m(U)p.m(G)p.m | |
| | | (U)p.[fl2r](C)p.m(U)p.[fl2r](A)p.m(G) | | (U)p.m(C)p.m(U)p.m(U)p.m(C) | |
| | | p.m(G)p.m(G)[ps].m(U)[ps].m(U) | | p.m(G)[ps].m(A)[ps].m(A)p.d(T) | |
| | | | | p.d(T) | |
| 20 | 5′_(dT)2 | [5vp_una2moe](U)[ps].[fl2r](U)[ps].m | 17 | [5am6_p_d](T)p.d(T)p.m(C)[ps].m | 130 |
| | 7f8s | (C)p.m(G)p.m(A)p.[fl2r](A)p.m(G) | | (C)[ps].m(C)p.m(C)p.m(U)p.m(A) | |
| | | p.m(A)p.m(C)p.m(A)p.m(G)p.m(U)p.m | | p.m(G)p.[fl2r](A)p.[fl2r](A)p.[fl2r] | |
| | | (U)p.[fl2r](C)p.m(U)p.[fl2r](A)p.m(G) | | (C)p.m(U)p.m(G)p.m(U)p.m(C) | |
| | | p.m(G)p.m(G)[ps].m(U)[ps].m(U) | | p.m(U)p.m(U)p.m(C)p.m(G)[ps].m | |
| | | | | (A)[ps].m(A) | |
| 21 | 5′ | [5vp_una2moe](U)[ps].[fl2r](U)[ps].m | 17 | [5am6_p_rc16](U)p.d(T)p.d(T)p.m | 131 |
| | C16_(dt)2 | (C)p.m(G)p.m(A)p.[fl2r](A)p.m(G) | | (C)[ps].m(C)[ps].m(C)p.m(C)p.m | |
| | 7f8s | p.m(A)p.m(C)p.m(A)p.m(G)p.m(U)p.m | | (U)p.m(A)p.m(G)p.[fl2r](A)p.[fl2r] | |
| | | (U)p.[f12r](C)p.m(U)p.[fl2r](A)p.m(G) | | (A)p.[fl2r](C)p.m(U)p.m(G)p.m | |
| | | p.m(G)p.m(G)[ps].m(U)[ps].m(U) | | (U)p.m(C)p.m(U)p.m(U)p.m(C) | |
| | | | | p.m(G)[ps].m(A)[ps].m(A) | |
| 22 | 3′ | [5vp_una2moe](U)[ps].[fl2r](U)[ps].m | 17 | [5am6_p_m](C)[ps].m(C)[ps].m(C) | 132 |
| | C16_(dt)2 | (C)p.m(G)p.m(A)p.[fl2r](A)p.m(G) | | p.m(C)p.m(U)p.m(A)p.m(G)p.[fl2r] | |
| | 7f8s | p.m(A)p.m(C)p.m(A)p.m(G)p.m(U)p.m | | (A)p.[fl2r](A)p.[fl2r](C)p.m(U) | |
| | | (U)p.[fl2r](C)p.m(U)p.[fl2r](A)p.m(G) | | p.m(G)p.m(U)p.m(C)p.m(U)p.m | |
| | | p.m(G)p.m(G)[ps].m(U)[ps].m(U) | | (U)p.m(C)p.m(G)[ps].m(A)[ps].m | |
| | | | | (A)p.d(T)p.d(T)p.[rc16](U) | |

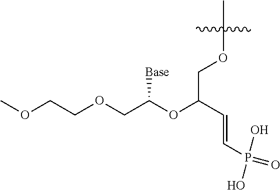

5vp_una2moe 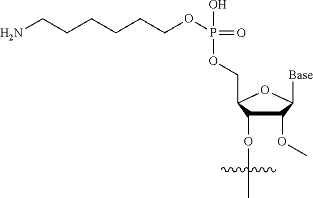
5am_6p_m 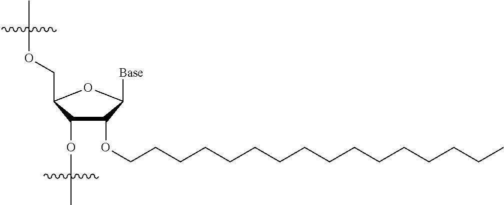
rc16

In Table 80, m indicates the 2′-O-methyl (2′-OMe) ribose modification; [fl2r] indicates the 2′-deoxy-2′-fluoro (2′-F) ribose modification; [ps] indicates phosphorothioate (PS) linkage; p indicates phosphodiester (PO) linkage; [rc16] indicates 2′O—C16 Phosphoramidite.

A 21-mer double-stranded siRNA with 19 bases of complementarity and 3′ dinucleotide overhangs was designed against mouse DMPK. The sequence (5′ to 3′) of the guide/antisense strand was UAAUACCGAGGAAUGUCGGUU (SEQ ID NO: 6). Base, sugar and phosphate modifications were used to optimize the potency of the duplex and reduce immunogenicity. All single-stranded polynucleic acid molecules were fully assembled on solid phase using standard phosphoramidite chemistry and purified over HPLC. Purified single-stranded polynucleic acid molecules were duplexed to get the double-stranded siRNA. The passenger strand contained a conjugation handle, a C6-NH2, at the 5′ end, which was connected to the siRNA passenger strand via phosphorothioate-inverted abasic-phosphorothioate linker.

### Synthesis of 5-Octadecynyl-2′-OMe-Uridine Phosphoramidite (20)

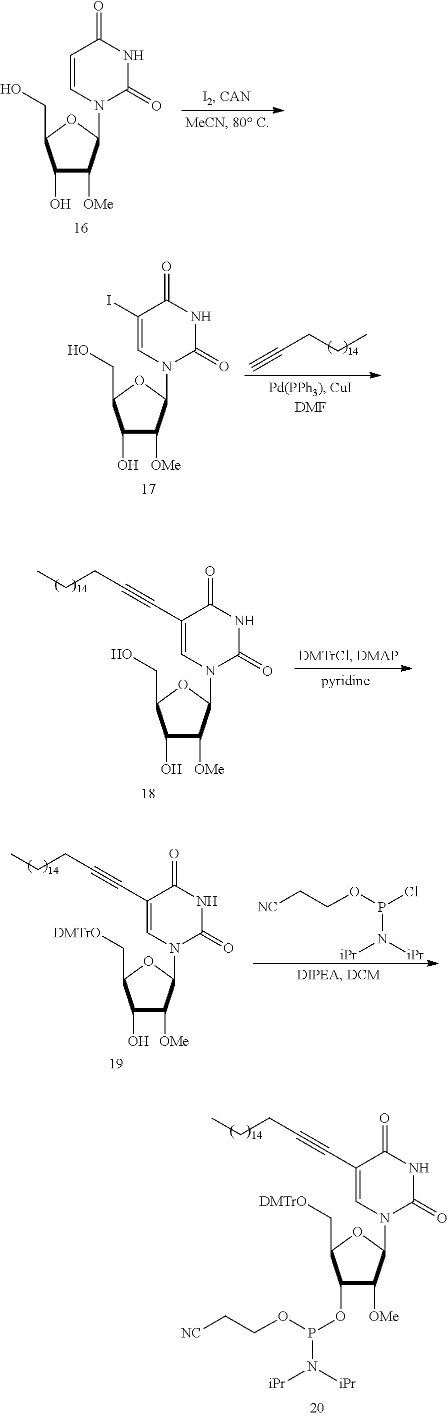

### Synthesis of Compound 17

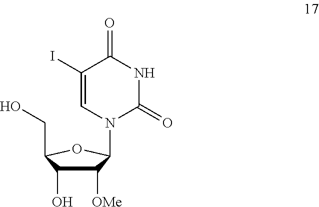

To a solution of compound 16 (2.0 g, 7.75 mmol, 1 equiv) in anhydrous MeCN (38 mL) was added iodine (1.18 g, 4.65 mmol, 0.6 equiv) and cerium (IV) ammonium nitrate (2.12 g, 3.87 mmol, 0.5 equiv). The resulting solution was stirred at 80° C. for 4 h, then cooled to 0° C. The resulting white precipitate was filtered and washed with MeCN (2×25 mL) and water (2×25 mL) to yield 17 (1.74 g, 60%) as a cream colored solid. LC-MS: calculated [M+Na]⁺=406.97, observed 407.00.

### Synthesis of Compound 18

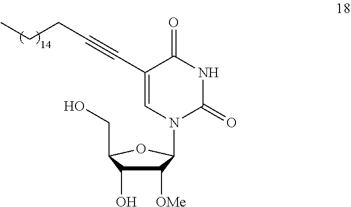

To a solution of compound 17 (1.0 g, 7.75 mmol, 1 equiv) in anhydrous DMF (13 mL) was added Pd(PPh₃)₄ (301 mg, 0.26 mmol, 10 mol %), CuI (99 mg, 0.52 mmol, 20 mol %), 1-octadecyne (1.3 g, 5.21 mmol, 2 equiv), and triethylamine (TEA, 0.73 mL, 5.21 mmol, 2 equiv). The resulting solution was stirred overnight at RT. After 20 h, reaction was heated to 60° C. for 1 h and monitored by LC-MS. Reaction mixture was cooled to rt and solvent was removed under reduced pressure. The resulting residue was taken up in EtOAc (30 mL) and washed with water (2×15 mL) and brine (2×15 mL). The combined organic extract was dried over MgSO₄, filtered, and concentrated under reduced pressure. The crude material was purified via CombiFlash® using silica gel chromatography (0-80% EtOAc/heptane, eluted at 50% EtOAc/heptane) to afford 18 (1.3 g, 98%) as a yellow solid. LC-MS: calculated [M+H]⁺=507.34, observed 507.36.

### Synthesis of Compound 19

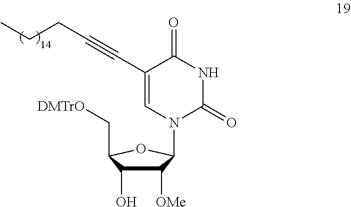

To a solution of compound 18 (1.0 g, 1.97 mmol, 1 equiv) in anhydrous pyridine (20 mL) was added DMAP (24 mg, 0.2 mmol, 10 mol %). Reaction mixture was cooled to 0° C., followed by addition of 4,4′-dimethoxytrityl chloride (DMTrCl, 1.0 g, 2.96 mmol, 1.5 equiv). The resulting solution was stirred overnight at RT. The reaction mixture was quenched with MeOH (5 mL) and concentrated under reduced pressure. The crude material was purified via CombiFlash® using silica gel chromatography (0-50% EtOAc/heptane, eluted at 40% EtOAc/heptane) to afford 19 (592 mg, 37%) as a yellow solid. LC-MS: calculated [M+Na]⁺=831.46, observed 831.45.

### Synthesis of Compound 20

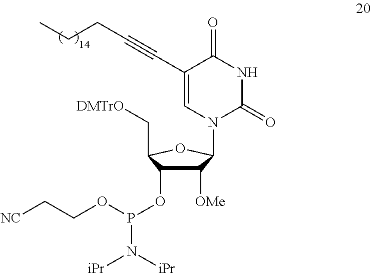

To a solution of compound 19 (407 mg, 0.50 mmol, 1 equiv) in anhydrous DCM (5 mL) at 0° C. was added DMAP (24 mg, 0.2 mmol, 10 mol %) and DIPEA (0.64 mL, 3.66 mmol, 5 equiv). Reaction mixture was cooled to 0° C., followed by dropwise addition of 2-cyanoethyl N,N-diisopropylchlorophosphoramidite (0.33 mL, 1.46 mmol, 2 equiv). The resulting solution was stirred at RT for 1 h. The reaction mixture was quenched with MeOH (0.5 mL) and washed with NaHCO_{3(aq)}(1×10 mL) and brine (1×10 mL). The combined organic extract was dried over Na₂SO₄, filtered, and concentrated under reduced pressure. The crude material was purified via CombiFlash® using silica gel chromatography (0-50% EtOAc/heptane+1% TEA, eluted at 50% EtOAc/heptane) to afford 20 (316 mg, 63%) as a white, foamy solid. ³¹P-NMR (202 MHz, CD₃CN) δ 150.41, 150.10; LC-MS: calculated [M+H]⁺=1009.58, observed 1009.56.

### Conjugate Synthesis and Characterization

The CD₇₁ Fab-siRNA conjugates were made, purified and characterized as described in Example 3. All conjugates were made through cysteine conjugation with a SMCC linker. Conjugates were characterized chromatographically as described in Table 81.

TABLE 81
HPLC Retention Time (RT) in Minutes

| | | RT, SAX |
|---|---|---|
| Group(s) | Conjugate | Method-5 |
| 1 | mR17-Fab-siDMPK DAR1 | 3.422 min |
| 2 | mR17-Fab- 20/21-mer 7f.8s degradable C22 Pos1 siDMPK DAR1 | 3.514 min |
| 3 | mR17-Fab- 20/21-mer 7f.8s degradable overhang MM C22 Pos1 | 3.525 min |
| | siDMPK DAR1 | |
| 4 | mR17-Fab- 5′ C22_(dt)2_7f8s siDMPK DAR1 | 3.588 min |
| 5 | mR17-Fab-20/21-mer 7f.8s degradable C18 5-U Pos1 siDMPK | 3.481 min |
| | DAR1 | |

TABLE 82
siRNA Sequences used in Example 27

| | | | SEQ | | SEQ |
|---|---|---|---|---|---|
| Compound | siRNA | | ID | | ID |
| No. | Ref. | siRNA Guide Sequence | NO: | siRNA Passenger Sequence | NO: |
| 1 | si_075_19bp | RNA1{[5vp_una2moe](U)[ps].[fl2r](A) | 44 | RNA1{[5am6_p_m](C)[ps].m(C) | 133 |
| | Parent | [ps].m(A)p.m(U)p.m(A)p.[fl2r](C)p.m | | [ps].m(G)p.m(A)p.m(C)p.m(A)p.[fl2r] | |
| | (+Vp) | (C)p.m(G)p.m(A)p.m(G)p.m(G)p.m(A) | | (U)p.[fl2r](U)p.[f12r](C)p.m(C) | |
| | | p.m(A)p.[fl2r](U)p.m(G)p.[fl2r](U)p.m | | p.m(U)p.m(C)p.m(G)p.m(G)p.m | |
| | | (C)p.m(G)p.m(G)[ps].m(U)[ps].m(U)} | | (U)p.m(A)p.m(U)[ps].m(U)[ps] | |
| | | $$$$V2.0 | | m(A)}$$$$V2.0 | |
| 2 | 20/21-mer | RNA1{[5vp_una2moe](U)[ps].[fl2r](A) | 44 | RNA1{[5am6_p_rc22](A)p.m(C) | 134 |
| | 7f.8s | [ps].m(A)p.m(U)p.m(A)p.[fl2r](C)p.m | | [ps].m(C)[ps].m(G)p.m(A)p.m(C) | |
| | degradable | (C)p.m(G)p.m(A)p.m(G)p.m(G)p.m(A) | | p.m(A)p.[fl2r](U)p.[fl2r](U)p.[fl2 | |
| | duplex, | p.m(A)p.[fl2r](U)p.m(G)p.[fl2r](U)p.m | | r](C)p.m(C)p.m(U)p.m(C)p.m(G) | |
| | C22 Pos1 | (C)p.m(G)p.m(G)[ps].m(U)[ps].m(U)} | | p.m(G)p.m(U)p.m(A)p.m(U)[ps].m | |
| | | $$$$V2.0 | | (U)[ps].m(A)}$$$$V2.0 | |
| 3 | 20/21-mer | [5vp_una2moe](U)[ps].[fl2r](A)[ps].m(A) | 12 | [5am6_p_rc22](U)p.m(C)[ps].m(C) | 135 |
| | 7f.8s | p.m(U)p.m(A)p.[fl2r](C)p.m(C)p.m(G) | | [ps].m(G)p.m(A)p.m(C)p.m(A) | |
| | degradable | p.m(A)p.m(G)p.m(G)p.m(A)p.m(A) | | p.[fl2r](U)p.[fl2r](U)p.[fl2r](C)p.m | |
| | overhang | p.[fl2r](U)p.m(G)p.[fl2r](U)p.m(C)p.m | | (C)p.m(U)p.m(C)p.m(G)p.m(G) | |
| | MM | (G)p.m(G)[ps].m(U)[ps].m(U) | | p.m(U)p.m(A)p.m(U)[ps].m(U)[ps].m | |
| | duplex, | | | (A) | |
| | C22 Pos1 | | | | |
| 4 | 5′ | [5vp_una2moe](U)[ps].[f12r](A)[ps].m(A) | 12 | [5am6_p_rc22](U)p.d(T)p.d(T)p.m | 136 |
| | C22_(dt)2 | p.m(U)p.m(A)p.[fl2r](C)p.m(C)p.m(G) | | (C)[ps].m(C)[ps].m(G)p.m(A)p.m | |
| | 7f8s duplex | p.m(A)p.m(G)p.m(G)p.m(A)p.m(A) | | (C)p.m(A)p.[fl2r](U)p.[fl2r](U) | |
| | | p.[fl2r](U)p.m(G)p.[fl2r](U)p.m(C)p.m | | p.[fl2r](C)p.m(C)p.m(U)p.m(C)p.m | |
| | | (G)p.m(G)[ps].m(U)[ps].m(U) | | (G)p.m(G)p.m(U)p.m(A)p.m(U) | |
| | | | | [ps].m(U)[ps].m(A) | |
| 5 | 20/21-mer | [5vp_una2moe](U)[ps].[fl2r](A)[ps].m(A) | 12 | [5am6_p_m]([C18_Alk_5U])p.m(C) | 137 |
| | 7f.8s | p.m(U)p.m(A)p.[fl2r](C)p.m(C)p.m(G) | | [ps].m(C)[ps].m(G)p.m(A)p.m(C) | |
| | degradable | p.m(A)p.m(G)p.m(G)p.m(A)p.m(A) | | p.m(A)p.[f12r](U)p.[fl2r](U)p.[fl2r] | |
| | C18 5-U | p.[fl2r](U)p.m(G)p.[fl2r](U)p.m(C)p.m | | (C)p.m(C)p.m(U)p.m(C)p.m(G) | |
| | Pos1duplex | (G)p.m(G)[ps].m(U)[ps].m(U) | | p.m(G)p.m(U)p.m(A)p.m(U)[ps].m | |
| | | | | (U)[ps].m(A) | |

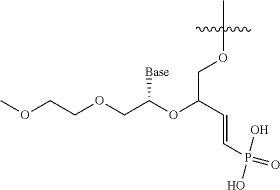

5vp_una2moe 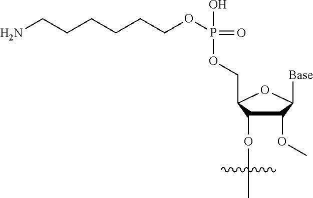
5am6_p_m 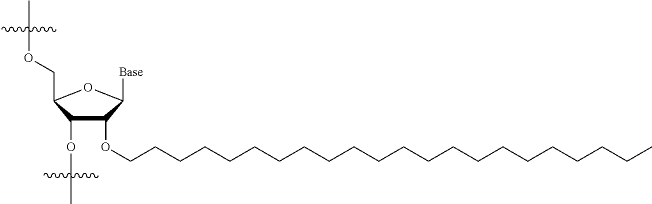
rc22 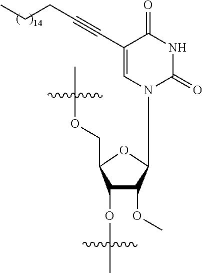
C18 5-U

In Table 82, m indicates the 2′-O-methyl (2′-OMe) ribose modification; [fl2r] indicates the '2′-deoxy-2′-fluoro (2′-F) ribose modification; [ps] indicates phosphorothioate (PS) linkage; p indicates phosphodiester (PO) linkage; [rc16] indicates the 2′O—C16 Phosphoramidite; [rc22] indicates the 2′O—C22 Phosphoramidite; [C18_Alk_5U] indicates the C18 5—U Phosphoramidite

### Results

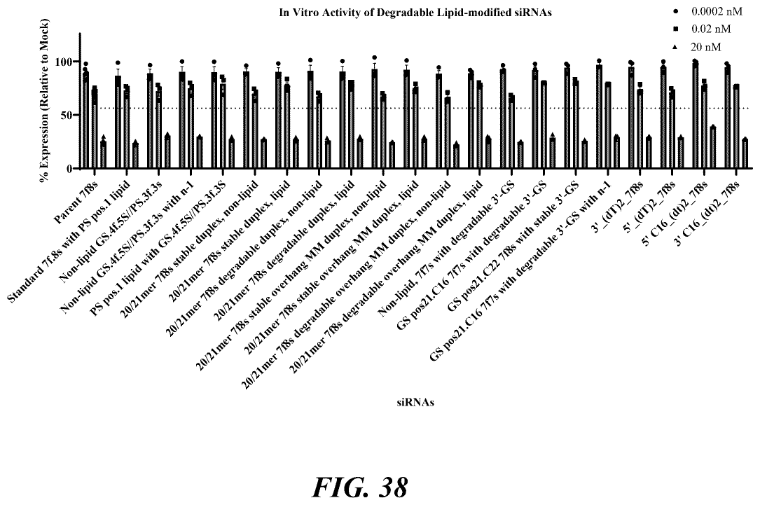

DMPK siRNA sequences containing C16 modifications were designed to be degradable/cleavable by enzyme-mediated degradation mechanisms including nucleases. They were assessed for their ability to knockdown DMPK mRNA in a luciferase-reporter assay in HEK293 cells. As shown in FIG. 38, the DMPK siRNA with C16 degradable modifications had similar activities compared to the ones of control DMPK siRNA or DMPK siRNA with non-degradable (stable) C16 modifications. Overall, degradable or stable C16 modifications had no effects on siRNA activities.

Finally, DAR1 FabOCs 20/21-mer 7f.8s degradable duplex, C22 Pos1, 20/21-mer 7f.8s degradable overhang MM duplex, C22 Pos1, 5′ C22_(dt)2_7f8s duplex, and 20/21-mer 7f.8s degradable C18 5—U Pos1 duplex sequences were synthesized and isolated by HPLC (see Table 81).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A polynucleotide conjugate comprising a binding moiety conjugated to a polynucleotide molecule, wherein the polynucleotide conjugate further comprises a lipophilic moiety.

2. The polynucleotide conjugate of claim 1, wherein the lipophilic moiety is a linear or branched alkyl group.

3. The polynucleotide conjugate of claim 2, wherein the linear alkyl group is selected from the group consisting of C6, C8, C10, C12, C14, C16, C18, C20, C22, C24 and C26 hydrocarbon chain.

4. The polynucleotide conjugate of claim 1, wherein the lipophilic moiety is conjugated to a nucleotide of the polynucleotide molecule, wherein the lipophilic moiety is conjugated to a sugar moiety of the nucleotide of the polynucleotide molecule at the 2′- or 3′-carbon or a modification made thereof.

5. The polynucleotide conjugate of claim 1, wherein the polynucleotide molecule is a single-stranded polynucleotide or a double-stranded polynucleotide, wherein the double-stranded polynucleotide is a siRNA comprising a guide strand and a passenger strand.

6. The polynucleotide conjugate of claim 5, wherein the lipophilic moiety is conjugated to the passenger strand, wherein the lipophilic moiety is conjugated to a nucleotide of the passenger strand at one of the positions 2-8 from the 5′ end.

7. The polynucleotide conjugate of claim 5, wherein the lipophilic moiety is conjugated to a nucleotide of the passenger strand at position 2, 3, or 6 from the 5′ end.

8. The polynucleotide conjugate of claim 5, wherein the lipophilic moiety is conjugated to the guide strand, wherein the lipophilic moiety is conjugated to a nucleotide of the guide strand at one of the positions 16-21 from the 5′ end.

9. The polynucleotide conjugate of claim 1, wherein the lipophilic moiety is conjugated to the nucleotide via a first linker.

10. The polynucleotide conjugate of claim 9, wherein the first linker is selected from the compound of Formula (A) or Formula (B): 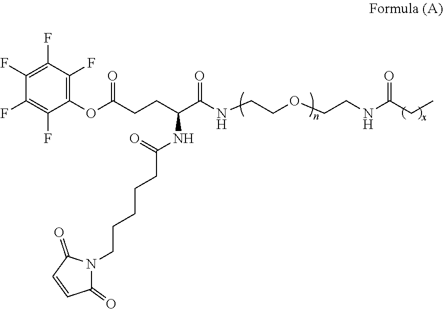
wherein n is 1-20; and x is 1-30; or 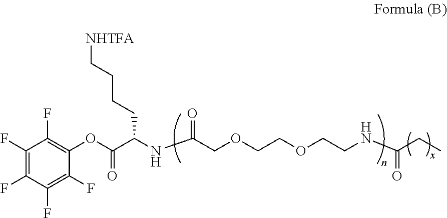
wherein n is 1-10; and x is 1-30.

11. The polynucleotide conjugate of claim 1, wherein the lipophilic moiety is further modified with a triazole (tz), an oleyl, a carboxylic acid, or a combination thereof.

12. The polynucleotide conjugate of claim 9, wherein the lipophilic moiety is conjugated to a second linker coupling the binding moiety to the polynucleotide molecule.

13. The polynucleotide conjugate of claim 12, wherein the second linker is a cleavable linker or a non-cleavable linker.

14. The polynucleotide conjugate of claim 12, wherein the second linker is a bond, C1-C30 alkyl group, a homobifunctional linker or a heterobifunctional linker, optionally conjugated to a C1-C6 alkyl group, a Bismal linker, SMCC linker, MBS linker, C16 linker, or C22 linker.

15. The polynucleotide conjugate of claim 1, wherein the lipophilic moiety is conjugated to the binding moiety.

16. The polynucleotide conjugate of claim 1, wherein the binding moiety is selected from peptides, toxins, sugars, carbohydrates, polymers, hormones, steroids, phospholipids, di- and triacylglycerols, fatty acids, hydrocarbons, enzyme substrates, biotin, digoxigenin, polysaccharides, a bicyclic peptide, a tricyclic peptide, or an antibody or antigen binding fragment thereof.

17. Use of a polynucleotide conjugate in a method of delivering a polynucleotide molecule to a tissue of a subject comprising administering the polynucleotide conjugate, wherein the polynucleotide conjugate comprises a binding moiety conjugated to a polynucleotide molecule, wherein the polynucleotide conjugate further comprises a lipophilic moiety.

18. Use of a polynucleotide conjugate in a method of treating muscle atrophy or myotonic dystrophy in a subject in need thereof comprising administering the polynucleotide conjugate, wherein the polynucleotide conjugate comprises a binding moiety conjugated to a polynucleotide molecule, wherein the polynucleotide conjugate further comprises a lipophilic moiety.

19. The method of claim 17, wherein the polynucleotide conjugate is delivered to a muscle tissue.

20. A pharmaceutical composition comprising the polynucleotide conjugate of claim 1 and a pharmaceutically acceptable excipient.
